(19) United States
(12) Patent Application Publication
(10) Pub. No.: US 2026/0015631 A1
(43) Pub. Date: Jan. 15, 2026

(54) TYPE I-D CRISPR-ASSOCIATED TRANSPOSASE AND TYROSINE RECOMBINASE TRANSPOSON SYSTEMS

(71) Applicants: The Broad Institute, Inc., Cambridge, MA (US); Massachusetts Institute of Technology, Cambridge, MA (US)

(72) Inventors: Guilhem Faure, Cambridge, MA (US); Makoto Saito, Cambridge, MA (US); Feng Zhang, Cambridge, MA (US)

(21) Appl. No.: 19/287,917

(22) Filed: Aug. 1, 2025

Related U.S. Application Data
(63) Continuation of application No. PCT/US2024/013963, filed on Feb. 1, 2024.
(60) Provisional application No. 63/442,710, filed on Feb. 1, 2023.

(51) Int. Cl. C12N 15/90; C12N 9/12; C12N 9/22; C12N 15/11

(52) U.S. Cl. CPC C12N 15/907; C12N 9/1241; C12N 9/226; C12N 15/11; C12N 15/902; C12N 2310/20; C12N 2800/90

(57) ABSTRACT

The invention provides for systems and methods for inserting large polynucleotides into precise locations in a target polynucleotide. In one aspect, the systems comprise an engineered Type I-D/Tn7 CRISPR-associated transposase system (CAST) comprising a Tn7-like transposase linked to or otherwise capable of associating with a Type I-D CRISPR-Cas complex (Tn7-CAST I-D). In another aspect, the systems comprise a Tn7-like transposase comprising a modular target site selection protein, called TnsF, that may be engineered to reprogram the Tn7-like transposase to facilitate insertion at different sites in a target polynucleotide. In another aspect, the systems comprise a transposon system comprising a tyrosine recombinase which provides for scar-less insertion of large donor sequences into target polynucleotides. Also provided are methods for modifying target polynucleotides using the systems; polynucleotides encoding the systems; delivery systems for delivering the components of the systems; and cells and biological products modified by or modified to include the systems.

### CROSS-REFERENCE TO RELATED APPLICATIONS

This application is a continuation application of PCT/US2024/013963, filed Feb. 1, 2024, which claims the benefit of U.S. Provisional Application No. 63/442,710, filed Feb. 1, 2023. The entire contents of the above-identified applications are hereby fully incorporated herein by reference.

### STATEMENT REGARDING FEDERALLY SPONSORED RESEARCH

This invention was made with government support under Grant No. (s) HG009761 awarded by the National Institutes of Health. The government has certain rights in the invention.

### SEQUENCE LISTING

The contents of the electronic sequence listing (BROD-5710US_ST26.xml, size is 10,707,377 bytes and it was created on Aug. 7, 2025) is herein incorporated by reference in its entirety.

### TECHNICAL FIELD

The subject matter disclosed herein is generally directed to systems, methods and compositions used for targeted gene modification, targeted insertion, perturbation of gene transcripts, and nucleic acid editing. Novel nucleic acid targeting systems comprise components of Clustered Regularly Interspaced Short Palindromic Repeats (CRISPR) systems and transposable elements.

### BACKGROUND

Recent advances in genome sequencing techniques and analysis methods have significantly accelerated the ability to catalog and map genetic factors associated with a diverse range of biological functions and diseases. Precise genome targeting technologies are needed to enable systematic reverse engineering of causal genetic variations by allowing selective perturbation of individual genetic elements, as well as to advance synthetic biology, biotechnological, and medical applications. Although genome-editing techniques such as designer zinc fingers, transcription activator-like effectors (TALEs), or homing meganucleases are available for producing targeted genome perturbations, there remains a need for new genome engineering technologies that employ novel strategies and molecular mechanisms and are affordable, easy to set up, scalable, and amenable to targeting multiple positions within the eukaryotic genome. This would provide a major resource for new applications in genome engineering and biotechnology.

The CRISPR-Cas systems of bacterial and archaeal adaptive immunity show extreme diversity of protein composition, genomic loci architecture, and system function. Systems comprising CRISPR-like components are widespread and continue to be discovered. Novel multi-subunit effector complexes and single-subunit effector modules may be developed as powerful genome engineering tools.

Citation or identification of any document in this application is not an admission that such a document is available as prior art to the present invention.

### SUMMARY OF THE INVENTION

The present disclosure provides an engineered system comprising one or more Type I-D Cas proteins; one or more CRISPR-associated Tn7 transposases or functional fragments thereof linked to or otherwise capable of associated with the one or more Type I-D Cas proteins; and a guide molecule capable of forming a complex with the one or more Type I-D Cas proteins and directing sequence-specific binding of the complex to the target polynucleotide.

In one embodiment, the one or more Type I-D Cas proteins comprise Cas5, Cas6, Cas7, and/or Cas10d. In another embodiment, the one or more Type I-D Cas proteins further comprise Cas1, Cas2, and/or Cas3d.

In one embodiment, the one or more CRISPR-associated Tn7 transposases comprise (i) TnsA, TnsB, and TnsC; or (ii) TnsAB and TnsC. In another embodiment, the one or more CRISPR-associated Tn7 transposases further comprise TniQ. In another embodiment, the one or more CRISPR-associated Tn7 transposases further comprise TnsE. In another embodiment, the one or more CRISPR-associated Tn7 transposases further comprise TnsF. In one embodiment, the one or more CRISPR-associated Tn7 transposases further comprise a first TniQ, and a second TniQ, wherein the first TniQ and the second TniQ are different. In another embodiment, TniQ is derived from a first species and the one or more Type I-D Cas proteins is derived from a second species different from the first species.

In one embodiment, the one or more CRISPR-associated Tn7 transposases are derived from a first species, and the one or more Type I-D Cas proteins are derived from a second species different from the first species.

In one embodiment, the target polynucleotide comprises a protospacer adjacent motif (PAM). In another embodiment, the PAM comprises the nucleotide sequence GTT. In one embodiment, the target polynucleotide comprises linear DNA, circular DNA, or genomic DNA.

In one embodiment, the engineered system further comprises a plurality of guide molecules capable of forming a complex with the one or more Type I-D Cas proteins and directing sequence specific binding of the complex to one or more target polynucleotides.

In one embodiment, the present disclosure provides an engineered system comprising one or more Tn7 transposases or functional fragments thereof comprising (a) TnsA, TnsB, TnsC, and TnsF; or (b) TnsAB, TnsC, and TnsF. In another embodiment, the engineered system further comprises TniQ.

In one embodiment, the engineered system further comprises one or more Cas proteins and a guide molecule capable of forming a complex with the one or more Cas proteins and directing sequence-specific binding of the complex to a target polynucleotide. In another embodiment, the one or more Cas proteins comprise a Type I, a Type II, or a Type V Cas protein. In another embodiment, the one or more Cas proteins further comprise a catalytically inactivated Cas protein. In another embodiment, the one or more Tn7 transposases are derived from a first species, and the one or more Cas proteins are derived from a second species different from the first species. In another embodiment, the engineered system further comprises a plurality of guide molecules capable of forming a complex with the one or more Cas proteins and directing sequence specific binding of the complex to one or more target polynucleotides.

In one embodiment, the present disclosure provides an engineered system comprising one or more tyrosine recombinases, one or more helix-turn-helix (HTH) domain proteins, and one or more TnsF homologs comprising a catalytic nuclease domain.

In one embodiment, the engineered system further comprises one or more Cas proteins and a guide molecule capable of forming a complex with the one or more Cas proteins and directing sequence-specific binding of the complex to a target polynucleotide. In another embodiment, the one or more Cas proteins comprise a Type I, a Type II, or a Type V Cas protein. In another embodiment, the one or more Cas proteins further comprise a catalytically inactivated Cas protein. In another embodiment, the engineered system further comprises a plurality of guide molecules capable of forming a complex with the one or more Cas proteins and directing sequence-specific binding of the complex to one or more target polynucleotides.

In one embodiment, the engineered system further comprises one or more GIY-YIG nucleases.

In one embodiment, the present disclosure provides a system comprising one or more polynucleotides encoding the components of any of the various embodiments of the engineered systems. In another embodiment, the system further comprises a donor polynucleotide. In another embodiment, the donor polynucleotide comprises a polynucleotide insert, a left element sequence, and a right element sequence.

In one embodiment, the present disclosure provides a vector comprising one or more polynucleotides encoding the components of any of the various embodiments of the engineered systems. In another embodiment, the vector further comprises a donor polynucleotide. In another embodiment, the donor polynucleotide comprises a polynucleotide insert, a left element sequence, and a right element sequence.

In one embodiment, the present disclosure provides an engineered cell comprising any of the various embodiments of the engineered systems or vectors. In another embodiment, the engineered cell produces and/or secretes an endogenous or non-endogenous biological product or chemical compound. In another embodiment, the biological product comprises a protein or an RNA. In one embodiment, the present disclosure provides a cell line comprising the engineered cell. In another embodiment, the present disclosure provides a composition comprising the engineered cell. In another embodiment, the composition is formulated for use as a therapeutic. In one embodiment, the present disclosure provides for a biological product or chemical compound produced by the engineered cell. In another embodiment, the biological product comprises a mutated protein or product provided by a template.

In one embodiment, the present disclosure provides an engineered cell or progeny thereof, engineered by use of any of the various embodiments of the engineered systems. In another embodiment, the cell or progeny thereof produces and/or secretes an endogenous or non-endogenous biological product or chemical compound. In another embodiment, the biological product comprises a protein or an RNA. In another embodiment, the engineered cell is further used as a therapeutic.

In one embodiment, the present disclosure provides a biological product or chemical compound produced by the engineered cell or progeny thereof. In another embodiment, the biological product comprises a protein or an RNA. In another embodiment, the protein comprises a mutation.

In one embodiment, the present disclosure provides a pharmaceutical composition for treatment of a disease or disorder, comprising the engineered cell or progeny thereof. In another embodiment, the treatment results in genetic changes in one or more cells. In another embodiment, the treatment results in correction of one or more defective genotypes. In another embodiment, the treatment results in improved phenotype.

In one embodiment, the present disclosure provides an engineered cell or progeny thereof comprising a mutation in the protein expressed from a gene comprising the target sequence. In another embodiment, the cell or progeny thereof comprises decreased transcription of a gene associated with the target sequence. In another embodiment, the cell or progeny thereof comprises increased transcription of a gene associated with the target sequence.

In one embodiment, the present disclosure provides a method of inserting a donor polynucleotide into a target polynucleotide in a cell, comprising introducing into the cell any of the various embodiments of the engineered systems. In one embodiment, the donor polynucleotide (a) introduces one or more mutations to the target polynucleotide; (b) corrects a premature stop codon in the target polynucleotide; (c) disrupts a splicing site; (d) restores a splicing site; or (e) a combination thereof. In another embodiment, the one or more mutations comprises substitutions, deletions, insertions, or a combination thereof. In another embodiment, the one or more mutations causes a shift in an open reading frame on the target polynucleotide. In one embodiment, the donor polynucleotide is between 100 bases and 30 kilobases in length. In one embodiment, insertion of the donor polynucleotide into the target polynucleotide in the cell results in (a) a cell or population of cells comprising altered expression levels of one or more gene products; or (b) a cell or population of cells that produces and/or secretes an endogenous or non-endogenous biological product or chemical compound. In another embodiment, the target polynucleotide comprises linear DNA, circular DNA, or genomic DNA.

In one embodiment, the present disclosure provides a method of inserting a donor polynucleotide into a target polynucleotide in a cell, wherein the one or more components of the engineered systems is expressed from a nucleic acid operably linked to a regulatory sequence. In another embodiment, the one or more components of the engineered system is introduced into a particle for delivery into a cell.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A-1C-Prediction of novel target selectors in Tn7-like transposons. (1A) Schematic of Tn7 transposition. TnsB (cyan) recognizes both ends (R, right and L, left) and excises the transposon with the help of TnsA (yellow). TnsD is a sequence specific target selector and binds an attachment site in the bacterial genome to recruit TnsC (orange) and the transposon for insertion. (1B) Pipeline for discovery of novel target selectors. Sequence databases were mined for Tn7 component seeds and searched for genomic co-localization of these seeds. The genomic neighborhoods of the detected loci were annotated, with the focus on cas effectors and genes that appear to be operonized with tns C and tniQ/tnsD. (1C) Locus architectures of known systems and novel systems identified in this study. Mu (muA and muB) and IS21 (istA and istB) encode relatives of TnsB and TnsC. IS21 has not been reported to be associated with a target selector. Tn7 encodes various target selectors including TniQ/TnsD, TnsE, and Cas effectors (Cas12k, Cascade I-F, and Cascade I-B, all of which partner with TniQ, the latter of which constitute CAST systems. Applicants identified a novel CAST system containing Cascade I-D and a novel target selector named TnsF. Applicants also identified a TnsF-like target selector in a distinct non-Tn7 transposon.

FIG. 2—Phylogenetic tree of TnsC homologs. Rings around the tree show the presence of a particular gene or a feature in the vicinity of tnsC within the genomic contig. From inner to outer ring: tnsA is shown in light pink, tnsAB fusion is shown in light blue, presence of tnsB operonized with the central tns C (representative of the leaf) is shown in cyan, presence of an additional distinct tns C operonized with tnsB in the vicinity is shown in dark blue, tniQ/tnsD is shown in light purple and the presence of a second tniQ/tnsD in dark purple where both their protein size are proportional to size of the ring bar, tnsE is shown in dark red, cas effectors and cas6 genes are shown in yellow, the presence of a gene operonized with the central tns C is shown in green. Various known transposons are annotated around the tree including known CAST systems. Red boxes highlight areas of interest. The Mu clade corresponds to the left branch harboring a conserved gene operonized with MuB (homologous to tns (*). This gene is part of the Mu phage genome. By subtraction, the Tn7 clade corresponds to the remaining clade and is characterized by the presence of TniQ/TnsD.

FIG. 3A-3C-Characterization of CAST I-D. (3A) Schematic of Cyanothece sp. PCC 7425 CAST I-D (CyCAST) locus architecture. (3B) RNA-guided insertion frequency of CyCAST into pTarget with PSP1, with or without TniQ and TnsD. (3C) Protein-mediated insertion frequency of CyCAST into pTarget with tRNA-leu, with or without TniQ and TnsD. ddPCR experiments were performed with three biological replicates. All data points are shown with an error bar showing standard deviation, and statistical significance was assessed by t-test.

FIG. 4A-4B-Comparison of dual TniQ-TnsD in CAST systems. (4A) Domain architecture comparison of TniQ/TnsD. Left: CAST TniQ-like proteins involved in RNA-guided transposition. Right: TnsD-like proteins involved in protein-guided transposition compared with E. coli canonical Tn7 TnsD. Except for CAST V-K TniQ, all TniQ/TnsD share a common core region composed of a N-terminal helical domain (HelD, a zinc finger (ZF), a helical linker, and a C-terminal helical domain (Hel2) (CAST I-D TniQ contains only a partial Hel2 domain). TnsD-like proteins performing protein-guided insertion in CASTs harbor long and diverse C-terminal regions folding into multiple HTH domains similar to Tn7 TnsD. (4B) Domain architecture of TniQ and TnsF in Tn6022 (left). Docking prediction of TniQ and TnsF (right). Pink in TniQ indicates C-terminal extension predicted to interact with TnsF. Pink and purple in TnsF indicate the predicted tandem core binding domain (CB1 and CB2) and the partial catalytic domain pCAT, respectively.

FIG. 5A-5E-Characterization of TnsF-containing Tn6022. (5A) Schematic of Acinetobacter johnsonii Tn6022 (AjTn6022) locus architecture. (5B) Insertion frequency of AjTn6022 into pTarget with a 200 bp-fragment of comM in the absence of indicated AjTn6022 component. (5C) Purified proteins used in the pull-down assay to capture the physical interaction between TnsF and TniQ (left). Eluted TnsF-TniQ complexes on the beads were analyzed on the protein gel (right). (5D) Molecular details of the interaction region from the docking prediction between Tn6022 TnsF (purple) and TniQ (pink). TniQ is predicted to interact via its C-terminal helix with the pCAT domain of TnsF. The interaction involves 2 salt bridges (circled in red) and multiple hydrophobic interactions (dashed green lines) (left). (5E) Insertion frequency of AjTn6022 into the comM gene fragment by TnsF and TniQ mutants. For TniQ mutants, overlapped TniQ and TnsF sequences were separated. ddPCR experiments were performed with three biological replicates. All data points are shown with an error bar showing standard deviation, and statistical significance was assessed by t-test.

FIG. 6A-6H—TnsF targets a conserved WalkerB motif in comM. (6A) Schematic of the locus architecture of Zoogloea sp. LCSB751 Target Selector based on tYrosine (Y) recombinase transposon (ZooTsy). (6B) Genetic requirement of YRec, HTH, and TnsF on ZooTsy transposition activity, as assayed by quantification of upstream-end1 junction formation by ddPCR. Deleted genes are indicated by a dashed outline. (6C) Insertion frequency of AjTn6022 into pTarget with a 200 bp-fragment of comM (left) and E. coli endogenous comM (right) in the absence of TnsF and/or presence of the pTarget (AjTn6022). pSC101 donor was used. (6D) Insertion frequency of ZooTsy into pTarget with a 200 bp-fragment of comM (left) and E. coli endogenous comM (right) in the absence of TnsF and/or presence of the pTarget (ZooTsy). pSC101 donor was used. (6E) Electrophoretic mobility shift assay to assess the interaction between a 200 bp- or 200 nt-fragment of AjcomM and purified AjTn6022-TnsF. (6F) Electrophoretic mobility shift assay to assess the interaction between a 200 bp- or 200 nt-fragment of ZoocomM and purified ZooTsy-TnsF_Y584F. (6G) (SEQ ID NO: 1-6) Insertion sites of Tn6022 and ZooTsy on E. coli endogenous comM and comM of their respective host used in the pTarget. ComM protein sequences (translated comM) are superimposed in frame to the nucleotide sequence. Pink rectangle indicate the genomic location of the walker B of the AAA ATPase encoded by comM, the red rectangle shows the probable hot spot binding region of both TnsFs. (6H) Model of TnsF target selection and insertions for Tn6022 and Tsy. comM is shown as a green arrow with the walker B genomic region indicated in red. ComM protein is shown below where walker B site is displayed. Purple arrows indicate the recognition of the similar attachment site by both TnsF (pink) while the dashed red arrows indicate the distinct insertion sites. Dashed gray indicates the recruitment of the transpososome to the insertion site. ddPCR experiments were performed with three biological replicates. All data points are shown with an error bar showing standard deviation, and statistical significance was assessed by t-test.

FIG. 7—Model of the evolution of the functional versatility of TniQ-based target selector. Evolutionary scenario for various Tn7-like transposons with distinct modes of target selection. Locus architecture is shown on the left and mechanics of target site selection on the right. (1) An ancestral Tn7-like transposon might have used TnsD for site-specific target selection and a DNA bending protein or complex (e.g., Cas effector, transcription factor, tyrosine recombinase) in trans as a second mode of target site selection. These DNA-bending proteins would create a distortion in the DNA that TnsC would recognize, albeit with a low efficiency. Gene duplication produced a second copy of TnsD. (2) Neofunctionalization of the second copy of TnsD yielded TniQ, which evolved to optimize the interaction between a trans DNA bending target selector and the target site. The trans system could also be captured by the transposon as cargo. (3) Further domestication of the DNA binding system would then occur, eventually leading to the loss of the native function of the system (e.g., CAST I-B), fusion to TniQ to create a new TnsD (e.g., TniQ-TnsF fusion), or adaptation of the system for dual modes of transposition as in CAST V-K, which relies entirely on the CRISPR system for both homing and jumping. Pink indicates DNA binding function; green indicates TniQ core; blue indicates native function of DNA binding system.

FIG. 8A-8F—(8A) CAST I-D loci architectures and comparison with PmcCAST I-B2 locus. Rectangles show predicted end, light gray genes indicate predicted homing genes. Cargo areas are summarized by a gray rectangle. Cascade components are shown in purple; dark for I-B2, light purple for I-D. Adaptation module and cas3d are show in lighter purple. CRISPR arrays are shown by dark grey vertical rectangle for repeats and dark purple diamonds for spacers. Ends are displayed via light grey vertical rectangle. tRNAs is the predicted homing gene. Tn7 components are shown in similar colors than in FIG. 1A-1C. Contigs and coordinates are indicated for CAST I-D loci. Absence of one of the locus coordinates indicates the edge of the contig. NCBI accessions of Cas10d proteins are written below each cas10d gene. (8B) (SEQ ID NO: 7-16) Cas10d subtree restricted to CAST I-D and close relatives. Local alignment of the HD nuclease catalytic sites is shown on the right of the tree. Presence of Tn7 components is shown on the right. Right, structural model of CyCAST Cas10d colored by domain architecture. The inset shows the detailed area of the catalytic pocket. (8C) Weblogo representation of PAM for CyCAST RNA-guided insertions. (8D) CyCAST RNA-guided insertion positions identified by deep sequencing with four different primer pairs. (8E) Docking predictions of CyCAST TnsC (coraD with TniQ (green) and TnsD (pink). Insets are zoom ins of two regions of interaction between TnsC with TniQ and TnsD. The C-terminal portion of the TniQ core region is disordered and truncated compared to the corresponding regions of CAST I-D TnsD and its relatives in CAST I-B2, suggesting that the interaction between TniQ and Cascade I-D is unstable and that TniQ serves as a facilitator rather than an essential scaffold for the interaction between Cascade I-D and TnsC. (8F) CyCAST-mediated prominent insertion location at tRNA-leu gene on target plasmid identified by deep sequencing.

FIG. 9A-9D-(9A) Structural comparison of Tn7 TnsD and CAST TniQ/TnsD proteins. CAST TniQ corresponds to the protein partnering with the Cas effector for RNA-guided transposition. Hell, ZF, and linker helix are conserved in all TniQ/TnsD and are colored using the same palette as in FIG. 3A-3B. Hel2 has two distinct folds, one colored in rainbow by secondary structure, has structural similarity to XRE T-F and is shared between Tn7 TnsD and CAST TniQ/TnsD from I-B and I-D, the other colored in red is found in CAST I-F TniQ/TnsD. The C-terminal regions of TniQ/TnsD are partially truncated for visualization in TniQ/TnsD harboring long extensions that are partially shown in pink. (9B) Phylogenetic tree of the core region of TniQ/TnsD (see Methods). Rings around the tree show the presence of a particular gene or a feature in the vicinity of tniQ/tnsD within the genomic contig. From inner to outer ring: TniQ/TnsD protein size is shown as a bar proportional to the length of TniQ/TnsD, presence of tnsE is shown in red, cas effectors and cas6 genes are shown in purple, the presence of a gene operonized with the tniQ/tnsD is shown in light green. Various known transposons are annotated around the tree including known CAST systems. Red boxes highlight areas of interest, and gene architectures of these systems are shown in panel D. Dual tniQ-tnsD are highlighted via a connector colored from a rainbow gradient starting on the left border and going to the right border of the tree where the color of the connector is set by the location of TniQ the most in the left. A connector originating and arriving within areas of the same color indicates the TniQ/TnsD tandem belong to closely related leaves. By contrast, a connector originating and arriving within areas of different colors indicates TniQ/TnsD distantly related. (9C) Left. Protein size distribution of TniQ/TnsD in single TniQ/TnsD systems (in red) and dual TniQ-TnsD systems (in green). From the 4 peaks, 4 groups (delineated by dashed lines) were defined: group1<220aa, group 2 220-400aa, group 3 400-550aa, and group 5>550aa. Right. Protein size comparison of TniQ/TnsD in dual TniQ-TnsD systems. Y axis indicates the protein size of the smaller of the TniQ/TnsD tandem; X axis indicates the size of the larger one. Solid lines segregate the different groups by protein size. Number of occurrences is indicated for each of the groups. (9D) Schematics of locus architecture for systems of interest (corresponding to systems boxed in red in panel C). One example of a system harboring divergent dual TniQ-TnsD in which TniQ and TnsD share less than 20% of sequence identity. Five examples of systems with conserved candidates (in green) operonized with a tniQ/tnsD. Below the first candidate is a docking prediction between the candidate (light green), TnsC (coraD and TnsD (dark green). TnsD is truncated for visualization purposes.

FIG. 10A-10D-(10A) TnsC homologs phylogenetic tree and presence of TnsE (in red). The 2 distinct branches with TnsE are represented by Tn7 E. coli (Tn7) and Tn6022. (10B) Locus architecture of Tn7 (top) and Tn6022 (bottom) encoding tnsk. Below each locus, the predicted domain architecture of their respective TnsE proteins is shown. Both TnsEs have 2 single strand DNA binding like domains (SSBa and SSBb) in the N-terminal region and a double strand DNA binding domain (DBD) in the C-terminal region that has been solved experimentally (PDB: 5D17). The SSBs are deduced from structural modelling and structural mining (panel C and D). (10C) Structural modeling of TnsE Tn7 and Tn6022 indicates similar domain architecture despite their low sequence similarity (8% sequence identity). (10D) Structural superimposition of TnsE N-terminal domains (pink and orange) with PriB (light and dark blue) for Tn7 TnsE (left) and Tn6022 TnsE (right), suggesting TnsE contains 2 SSB domains. The second domain is split into 3 regions separated by a linker (yellow) and a domain of unknown function.

FIG. 11A-11F-(11A) Structural comparison of CB1, CB2, and pCAT domain of TnsF with the homodimer tyrosine recombinase (Yrec) XerH structure bound to DNA. Monomer of Yrec contains one CB and one CAT (monomers are circled in dashed red lines). Each CB and CAT of Yrec are bound to the attachment site and dimerize with the same domain of another monomer of Yrec. Structural similarity between CB and CAT of Yrec and CB and pCAT of TnsF is shown via matching secondary structural colors. Gray colored regions are found only in Yrec and not in TnsF. (11B) Domain architecture comparison between AjTnsF (Tn6022) and ZooTnsF (Tsy). The domain architectures were deduced from structural prediction of both proteins. Both TnsF proteins encode an N-terminal zinc finger region (ZFs, blue), a dual core binding domain (CB1 and CB2, pink), and a partial catalytic domain in AjTnsF (pCAT in purple) and complete catalytic domain in ZooTnsF (CAT in purple and orange). Tyrosine catalytic position is annotated in ZooTnsF and in the structure. (11C) Nanopore long-read sequencing to characterize the structure of pInsert. (11D) (SEQ ID NO: 17-18) Sanger sequencing chromatograms for RE and LE junctions of a simple insertion at Tn6022 attachment site in comM 100 bp-fragment on pTarget. “CCCGC” is a target site duplication (TSD). (11E) Phylogenetic tree built from TnsF homologs extracted from the CLANS analysis (panel A). Rings around the tree show the presence of a particular gene or a feature in the vicinity of tns/within the genomic contig. From inner to outer ring: presence of comM fragments in the vicinity of tns/in dark purple, presence of a tyrosine recombinase gene (yrec) in light purple, presence of tniQ in green, and presence of gene encoding a GIY-YIG nuclease in light blue. TnsF protein size is shown in pink as a bar proportional to its length. (11F) Structural homology between TnsF, RitB, and protelomerase TelA and TelK. TnsF, RitB, and TelA were obtained using Alphafold2. TelK is an experimental structure and shows a dimer of TelK binding DNA (pdb: 2v6e). Colors are similar to panel B and indicate the various domains. The second TelK in the structure is shown in green. All these structural homologs encode 2 CB tandem domains and one CAT domain. TelK has an additional domain in C-terminal (gray). Only TnsF has a N-terminal with multiple zinc fingers.

FIG. 12A-121—(12A) (SEQ ID NO: 19) Experimental scheme for ZooTsy transposition assay. Left top: Schematic of the ZooTsy donor and target comM fragment for transposition assay in E. coli. Left bottom: Representative PCR amplicon images on the gel for each upstream-end1, downstream-end2, and circularized donor junction. pHelper has all Tsy components (YRec, HTH, TnsF, and GIY-YIG). Right top: Schematic of the Tsy circular intermediate isolation assay with a lacZα backbone pDonor to isolate circularized donor (cd). E. coli were transformed with pHelper and lacZα donor. A day after, plasmids were prepped, and used for re-transformation. The transformants were plated on the blue/white selection plate. Representative images of E. coli colonies on the blue/white selection plate. Right middle: The left shows the E. coli transformed with the mini-prep product after pHelper expression and the one transformed with the product after no pHelper expression. The middle image shows a representative gel image to show the original lacZα donor and isolated circularized donor (cd) after linearization by NruI restriction enzyme. The right shows the structure of the circularized donor (cd) as characterized by nanopore long-read sequencing. Right bottom: Sanger sequencing chromatograms for endl and end2 junctions of the circularized donor (cd). (12B) Schematic of transposition assay with R6K origin pDonor to isolate pInsert for nanopore long-read sequencing. (12C) Schematic of nanopore sequencing reads analysis pipeline. (12D) Structure of pInsert as characterized by nanopore long-read sequencing. Left: Nanopore sequencing reads mapping on the reference sequence of a ZooTsy simple insertion on pTarget. Right: Nanopore sequencing reads mapping on the reference sequence of a ZooTsy excised cointegrate on pTarget. (12E) Determinants of donor endl for ZooTsy transposition. Top: Schematic of the expected PCR amplicons from the upstream-end1 junction of simple insertions and excised cointegrates. Bottom left: A representative gel image showing PCR amplicons from the upstream-end1 junction. Four donor constructs with different end1 lengths (hom1: 12 bp, end1: 0, 35, 85 and 135 bp, end2: 39 bp, hom2: 12 bp) were tested. Purple arrow indicates the amplicons from simple insertions; dark blue arrow indicates amplicons from excised cointegrates. Bottom right: Simple insertion ratio (%) in total insertion reads quantified by nanopore sequencing. (12F) Top: Determinants of donor end 1 for ZooTsy transposition. Representative gel images of PCR amplicons from the upstream-end1 (u-end1), downstream-end2 (d-end2), and circularized donor (cd) junctions. Seven donor constructs with different end1 lengths (hom1: 12 bp, end1: 135-85 bp, end2: 39 bp, hom2: 12 bp) were tested. Bottom: Determinants of donor end2 for ZooTsy transposition. Representative gel images of PCR amplicons from the u-end1, d-end2, and cd junctions. Seven donor constructs with different end2 lengths (hom 1:12 bp, end1: 135 bp, end2: 39-0 bp, hom2: 12 bp) were tested. (12G) Donor sequence determinants for ZooTsy transposition. Top: Schematic of ZooTsy donor. Bottom: Representative gel images showing PCR amplicons from the u-end1, ds-end2, and cd junctions. Sixteen donors constructed by systematic combinations of four elements (hom1, end1, end2 and hom2) were tested. (12H) Requirements of tyrosine recombinase activities of YRec and TnsF on ZooTsy transposition activity. Quantification of u-end1 junction formation by ddPCR. Experiments were performed with three biological replicates. All data points are shown with an error bar showing standard deviation, and statistical significance was assessed by t-test. (12I) Representative gel images to show PCR amplicons from each junction (see FIG. 6B).

FIG. 13A-13D-(13A) AjTn6022 TTISS analysis in the absence of TnsF and/or presence of the pTarget. Filtered reads are mapped on CP011113.2 (Strain RR1, HB101 RecA+), complete genome for HB101-derived Endura competent cells and pTarget (AjTn6022) sequence. (13B) ZooTsy TTISS analysis in the absence of TnsF and/or presence of the pTarget. Filtered reads are mapped on CP011113.2 (Strain RR1, HB101 RecA+), complete genome for HB101-derived Endura competent cells and pTarget (ZooTsy) sequence. (13C) Insertion frequency of AjTn6022 into each comM gene fragment on pTarget. Four different comM fragments were tested. U50D50 is the fragment containing 50 bp upstream and 50 bp downstream of the insertion site on comM. U25D50 contains 25 bp upstream and 50 bp downstream of the insertion site. U50D25 contains 50 bp upstream and 25 bp downstream of the insertion site. U0D0 has no comM fragment. (13D) Insertion frequency of ZooTsy into each comM gene fragment on pTarget. Twenty-two different comM fragments were tested. U100-OD100 are the fragments containing 100-0 bp upstream and 100 bp downstream of the insertion site on comM. U0D0 has no comM fragment. Left: Quantification of u-end1 junction formation by ddPCR. All data points are shown with an error bar showing standard deviation, and statistical significance was assessed by t-test.

FIG. 14A-14D-(14A) Electrophoretic mobility shift assay to assess the interaction between a 200 bp-Aj comM fragment with 10 bp-mutation in the TnsF binding motif and purified AjTn6022-TnsF. (14B) Electrophoretic mobility shift assay to assess the interaction between a 200 bp- or 200 nt-Zoo comM fragment with 10 bp-mutation in the TnsF binding motif and purified ZooTsy-TnsF_Y584F. (14C) (SEQ ID NO: 20-26) Insertion frequency of AjTn6022 into pTarget with a 200 bp-fragment of Aj comM harboring 10 bp-mutation in the TnsF binding motif. (14D) (SEQ ID NO: 20-26) Insertion frequency of ZooTsy into pTarget with a 200 bp-fragment of Zoo comM harboring 10 bp-mutation in the TnsF binding motif. All data points are shown with an error bar showing standard deviation, and statistical significance was assessed by t-test.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2nd edition (1989) (Sambrook, Fritsch, and Maniatis); Molecular Cloning: A Laboratory Manual, 4th edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M.J. MacPherson, B.D. Hames, and G. R. Taylor eds.): Antibodies, A Laboratory Manual (1988) (Harlow and Lane, eds.): Antibodies A Laboratory Manual, 2nd edition 2013 (E.A. Greenfield ed.); Animal Cell Culture (1987) (R.I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oiD, semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

The present disclose provides gene editing systems capable of inserting large polynucleotides into precise locations in a target polynucleotide without requiring single or double strand breaks in the target DNA. The systems can be configured to insert large donor sequences into the target DNA. For example, the gene editing systems can be used to insert additional copies of a gene, replace dysfunctional copies of a gene, or insert donor polynucleotides edited to make multiple modifications at multiple sites in the donor polynucleotide, which is useful, for example, where multiple deleterious mutations need to be corrected within a given stretch of DNA. The gene editing systems are reprogrammable and can be configured to target insertions at different locations within the target DNA. In one aspect, the gene editing tool is an engineered Type I-D/Tn7 CRISPR-associated transposase system (CAST) comprising a Tn7-like transposase that is linked to or otherwise capable of associating with a Type I-D CRISPR-Cas complex (Tn7-CAST 1-D). In one aspect, the disclosure advantageously provides a Tn7-CAST 1-D system requiring a minimal Tn7 component that reduces the size and number of components needed to create a functional Tn7-CAST 1-D system. In another aspect, the present disclosure comprises a novel Tn7-like transposase that comprises a novel guided target selector called TnsF. The TnsF is a modular target site selection protein that may be engineered to reprogram the Tn7-like transposase to facilitate insertion at different target sites in a target DNA polynucleotide. In another aspect, the present disclosure provides a novel transposon system that comprises a tyrosine recombinase which provides for scar-less insertion of large donor sequences into target DNAs. In another aspect, the disclose provides methods for modify target DNA polynucleotides using the aforementioned gene editing systems. In another aspect, the present disclosure provides polynucleotides encoding said gene editing systems as well as delivery systems for delivering polynucleotide and polypeptide components of said systems. In another aspect, the present disclosure provides cells and biological products modified by or modified to include said gene editing systems.

The present disclosure provides engineered systems and methods for inserting a polynucleotide to a desired position in a target nucleic acid. In one aspect, the engineered systems comprise one or more Type I-D Cas proteins (e.g., Cas5, Cas6, Cas7, and/or Cas10d; and Cas1, Cas2, and/or Cas3d); one or more CRISPR-associated Tn7 transposases or functional fragments thereof linked to or otherwise capable of associating with the one or more Type I-D Cas proteins (e.g., TnsA, TnsB, and TnsC; or TnsAB and TnsC; TniQ; TnsE); and a guide molecule capable of forming a complex with the one or more Type I-D Cas proteins and directing sequence-specific binding of the complex to a target polynucleotide.

In another aspect, the engineered systems comprise one or more Tn7 transposases or functional fragments thereof (e.g., TnsA, TnsB, and TnsC; or TnsAB and TnsC; TniQ and one or more Tn7 target selector proteins (e.g., TnsF). In another embodiment, the engineered systems further comprise one or more Cas proteins (e.g., a Type I, a Type II, or a Type V Cas protein) and a guide molecule capable of forming a complex with the one or more Cas proteins and directing sequence-specific binding of the complex to a target polynucleotide.

In another aspect, the engineered systems comprise one or more tyrosine recombinases, one or more helix-turn-helix (HTH) domain proteins, and one or more TnsF homologs comprising a catalytic nuclease domain. In another embodiment, the engineered systems further comprise one or more Cas proteins (e.g., a Type I, a Type II, or a Type V Cas protein) and a guide molecule capable of forming a complex with the one or more Cas proteins and directing sequence-specific binding of the complex to a target polynucleotide. In another embodiment, the engineered systems further comprise one or more GIY-YIG nucleases.

The present disclosure also includes polynucleotides encoding components of the nucleic acid targeting systems, and vector systems comprising one or more vectors comprising said polynucleotide. The present disclosure also includes engineered cells, tissues, organs, organisms, biological products, pharmaceutical compositions comprising the systems or generated using the systems.

### Tn7-Cast I-D Systems

In one example embodiment, the gene editing system is a Tn7-CAST I-D system comprising a Tn7-like transposase linked to or otherwise capable of associating with a CRISPR-Cas Type I-D complex. Tn7-like transposes are multimeric proteins comprising multiple sub-units. Cas Type I-D is also a multimeric protein (also referred to as the Cascade complex) and further comprises a nucleic acid component, or guide molecule, capable of forming a complex with the Cas I-D multimeric protein and directing sequence specific binding of the complex to a target sequence in a target polynucleotide. The guide component comprises a guide sequence (also referred to as a “spacer”) and a scaffold. The spacer facilitates sequence-specific binding to the target sequence and the scaffold primarily facilitates complex formation with the Type I-D Cas protein. The systems disclosed herein are engineered such that the Tn7-CAST 1-D systems recognize and modify target sequences other than the target sequence of a naturally occurring Tn7-CAST 1-D system. This can be achieved, for example, by modifying the spacer sequence to target a target sequence other than the target sequence of the naturally occurring CRISPR-Cas I-D complex, modifying the scaffold, modifying the CRISPR-Cas Type I-D Cascade complex, modifying the Tn7-like transposase, or a combination thereof. The separate components of Tn7-CAST 1-D systems are discussed in further detail below.

### Type 1-D CRISPR-Cas

The Class I, Type I-D CRISPR-Cas comprises a protein and nucleic acid component. The protein component is a multimeric protein comprised of multiple subunits. The nucleic acid component, or guide molecule, is capable of forming a complex with the multimeric protein component and directing sequence-specific binding of the complex to a target polynucleotide.

### Type I-D CRISPR-Cas Protein Component

An example Type I-D Cas protein may comprise one or more of the following polypeptide subunits, Cas5, Cas6, Cas7, Cas10d, Cas3d, Cas1, and Cas2. In one example embodiment, the Type I-D Cas protein comprises one or more of Cas5, Cas6, Cas7, and Cas10d. In one example embodiment, the Type I-D Cas protein comprises Cas5, Cas6, Cas7, and Cas10d. Cas5, Cas6 and Cas7 may form a backbone structure of the Type I-D Cas protein and necessary for guide molecule and target polynucleotide interactions. Cas6 of the present Type I-D Cas proteins has not been previously reported as associated with Type I-D CRISPR-Cas systems. See e.g. Lin et al. “DNA targeting by subtype I-D CRISPR-Cas shows type I and type III features” Nucleic Acids Res. 2020; 48 (18): 10470-10478. In one example embodiment, the Type I-D Cas protein may comprise multiple copies of Cas5 in the backbone structure. Cas10d comprises an endonuclease HD domain and may also comprise a PAM recognition domain. Cas3d, when present, comprises a helicase domain that may unwind double-stranded target DNA. In one example embodiment, the Type I-D Cas protein does not comprise a Cas5. In one example embodiment, the Type I-D Cas protein does not comprise a Cas6. In one example embodiment, the Type I-D Cas protein does not comprise a Cas7. In one example embodiment, the Cas10d protein may be catalytically inactive or be engineered to be catalytically inactive.

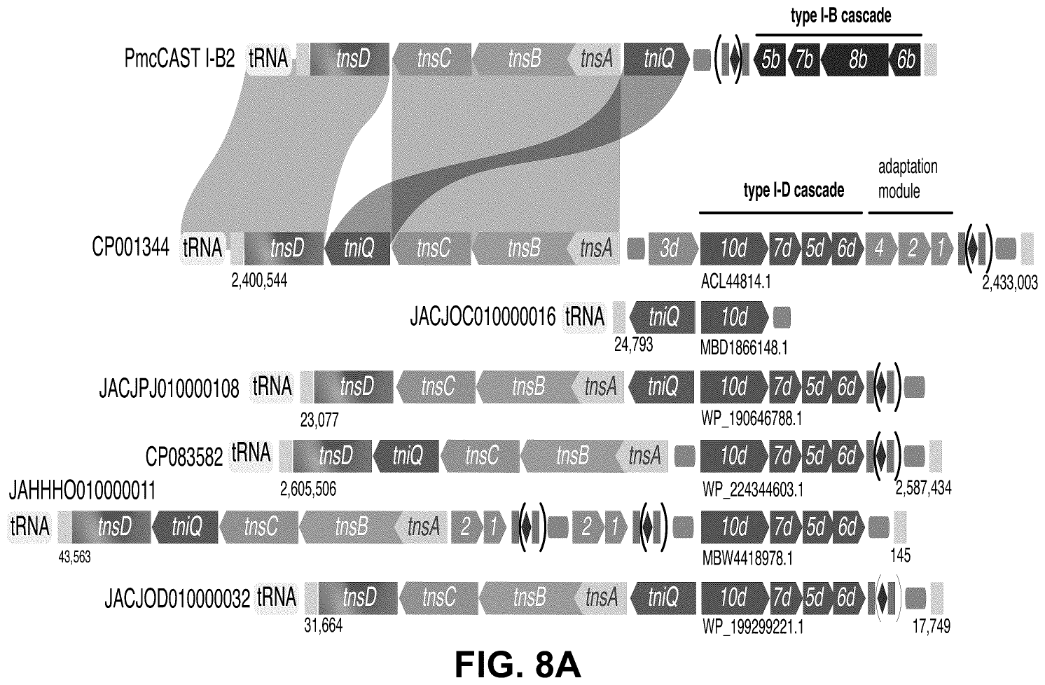

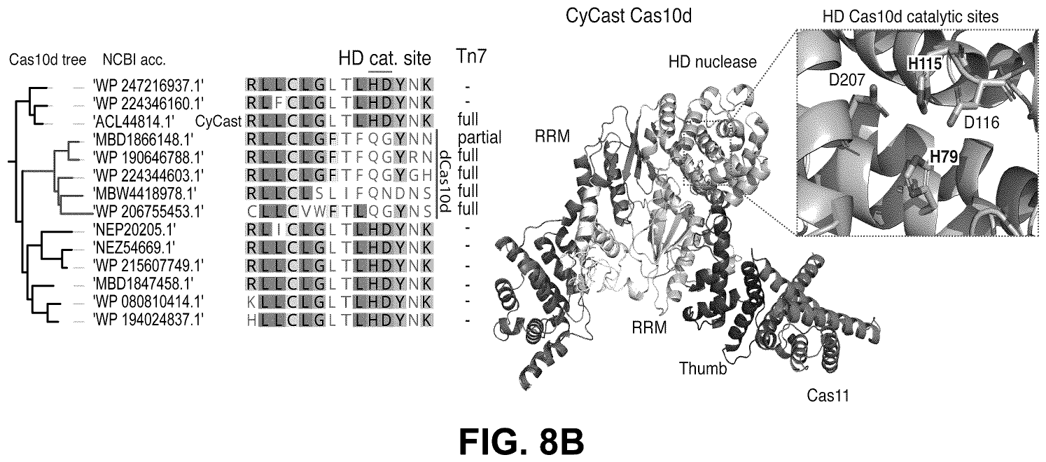

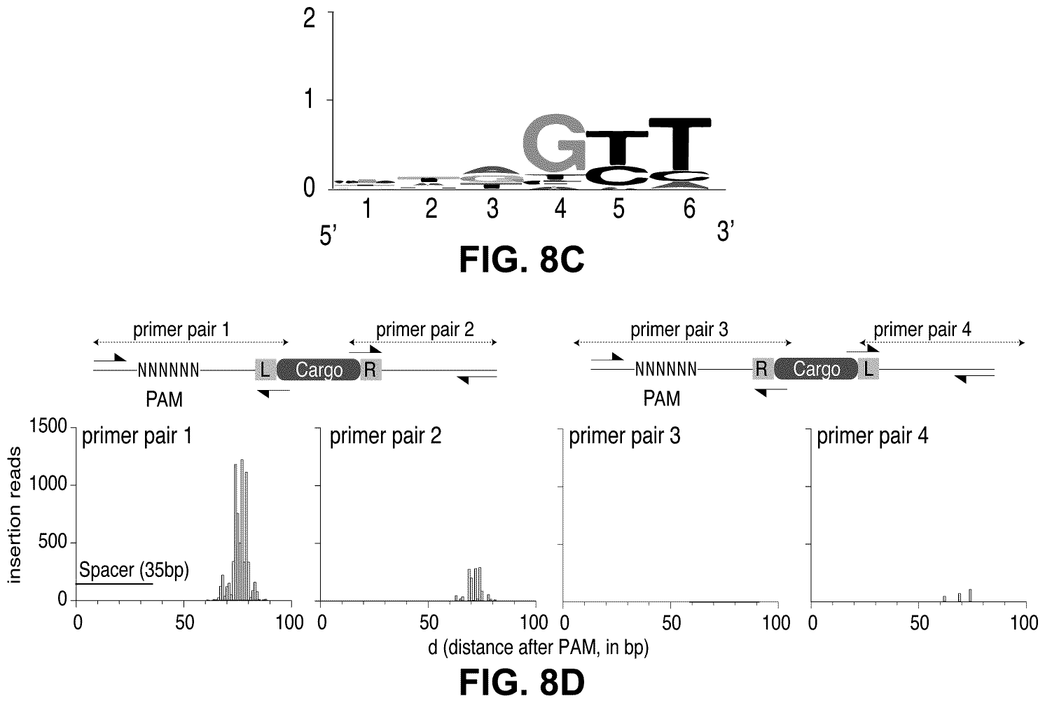

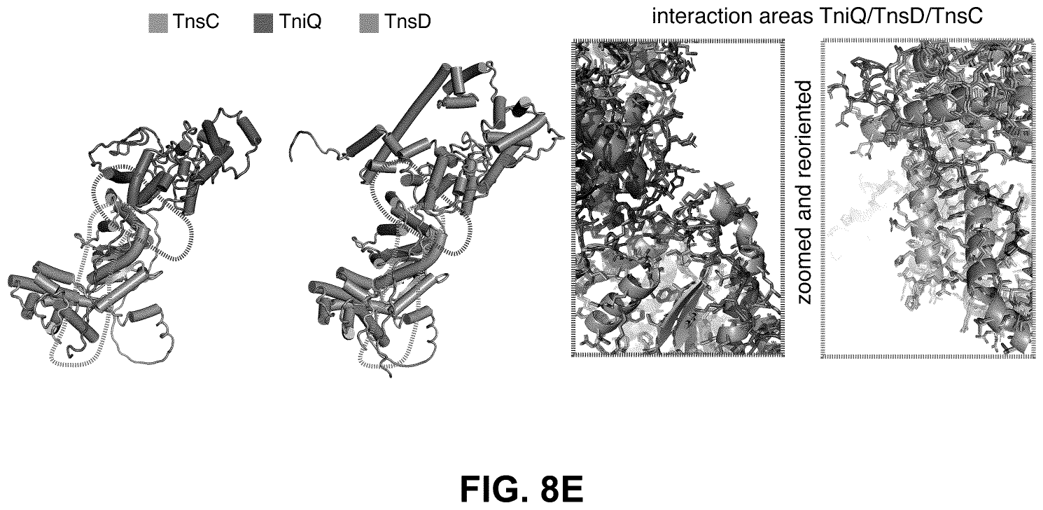

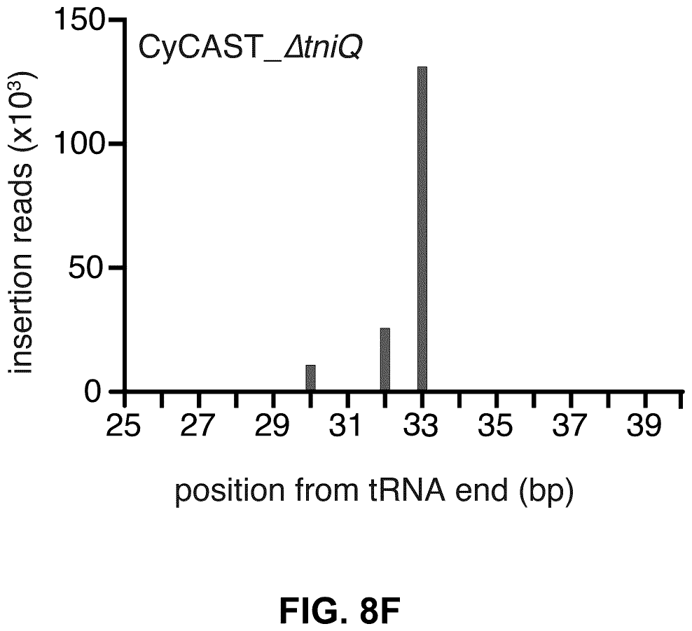

Example Type I-D Cas loci for Cas5, Cas6, Cas7, and Cas10d are provided in the CAST I-D loci illustrated in FIG. 8A. In an example embodiment, the one or more Type I-D Cas proteins are derived from the CAST I-D system of Cyanothece sp. PCC 7425. In a particular embodiment, the one or more Type I-D Cas proteins is a Cas10d derived from Cyanothece sp. PCC 7425 (ACL44814.1). Other example Cas 10d proteins are provided in FIG. 8A-8B and Table 5.

In some cases, the Cas proteins may be orthologs or homologs of the above mentioned Cas proteins. The terms “ortholog” and “homolog” are well known in the art. By means of further guidance, a “homolog” of a protein as used herein is a protein of the same species which performs the same or a similar function as the protein it is a homologue of. Homologous proteins may but need not be structurally related or are only partially structurally related. An “ortholog” of a protein as used herein is a protein of a different species which performs the same or a similar function as the protein it is an orthologue of. Orthologous proteins may but need not be structurally related or are only partially structurally related.

### Guide Molecules

The system herein may comprise one or more guide molecules. The term guide molecule may also be referred to a guide RNA (gRNA). A guide molecule comprises a guide sequence and a scaffold. The guide sequence may also be referred to herein as a “spacer sequence” When the spacer and scaffold are part of the same single molecule the molecular may be referred to as a single guide molecule or single guide RNA (sgRNA). In some cases, the system comprises one guide molecule. In certain cases, the system comprises a plurality of guide molecules. The guide molecule(s) may direct or may be capable of directing the binding of a guide-Cas protein complex to one or more target polynucleotides. For example, the system herein may be used for inserting a donor polynucleotide to one or more desired target sites with the direction of the guide molecule(s).

The guide molecule(s) may be component(s) of the CRISPR-Cas system herein. As used herein, the term “guide sequence” and “spacer” in the context of a CRISPR-Cas system, comprises any polynucleotide sequence having sufficient complementarity with a target nucleic acid sequence to hybridize with the target nucleic acid sequence and direct sequence-specific binding of a nucleic acid-targeting complex to the target nucleic acid sequence. The guide sequences made using the methods disclosed herein may be a full-length guide sequence, a truncated guide sequence, a full-length sgRNA sequence, or a truncated sgRNA sequence. In some embodiments, the degree of complementarity of the guide sequence to a given target sequence, when optimally aligned using a suitable alignment algorithm, is about or more than about 50%, 60%, 75%, 80%, 85%, 90%, 95%, 97.5%, 99%, or more. In certain example embodiments, the guide molecule comprises a guide sequence that may be designed to have at least one mismatch with the target sequence, such that an RNA duplex formed between the guide sequence and the target sequence. Accordingly, the degree of complementarity is preferably less than 99%. For instance, where the guide sequence consists of 24 nucleotides, the degree of complementarity is more particularly about 96% or less. In particular embodiments, the guide sequence is designed to have a stretch of two or more adjacent mismatching nucleotides, such that the degree of complementarity over the entire guide sequence is further reduced. For instance, where the guide sequence consists of 24 nucleotides, the degree of complementarity is more particularly about 96% or less, more particularly, about 92% or less, more particularly about 88% or less, more particularly about 84% or less, more particularly about 80% or less, more particularly about 76% or less, more particularly about 72% or less, depending on whether the stretch of two or more mismatching nucleotides encompasses 2, 3, 4, 5, 6 or 7 nucleotides, etc. In some embodiments, aside from the stretch of one or more mismatching nucleotides, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), Clustal W, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net). The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay as described herein. Similarly, cleavage of a target nucleic acid sequence (or a sequence in the vicinity thereof) may be evaluated in a test tube by providing the target nucleic acid sequence, components of a nucleic acid-targeting complex, including the guide sequence to be tested and a control guide sequence different from the test guide sequence, and comparing binding or rate of cleavage at or in the vicinity of the target sequence between the test and control guide sequence reactions. Other assays are possible, and will occur to those skilled in the art. A guide sequence, and hence a nucleic acid-targeting guide RNA may be selected to target any target nucleic acid sequence.

In certain embodiments, the guide sequence or spacer length of the guide molecules is from 15 to 50 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27-30 nt, e.g., 27, 28, 29, or 30 nt, from 30-35 nt, e.g., 30, 31, 32, 33, 34, or 35 nt, or 35 nt or longer. In certain example embodiment, the guide sequence is 15, 16, 17,18, 19, 20, 21, 22, 23, 24, 25, 26, 27, 28, 29, 30, 31, 32, 33, 34, 35, 36, 37, 38, 39 40, 41, 42, 43, 44, 45, 46, 47 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nt.

In some embodiments, the guide sequence is an RNA sequence of between 10 to 50 nt in length, but more particularly of about 20-30 nt advantageously about 20 nt, 23-25 nt or 24 nt. The guide sequence is selected so as to ensure that it hybridizes to the target sequence. This is described more in detail below. Selection can encompass further steps which increase efficacy and specificity.

In some embodiments, the guide sequence has a canonical length (e.g., about 15-30 nt) is used to hybridize with the target RNA or DNA. In some embodiments, a guide molecule is longer than the canonical length (e.g., >30 nt) is used to hybridize with the target RNA or DNA, such that a region of the guide sequence hybridizes with a region of the RNA or DNA strand outside of the Cas-guide target complex. This can be of interest where additional modifications, such deamination of nucleotides is of interest. In alternative embodiments, it is of interest to maintain the limitation of the canonical guide sequence length.

In some embodiments, the sequence of the guide molecule (direct repeat and/or spacer) is selected to reduce the degree secondary structure within the guide molecule. In some embodiments, about or less than about 75%, 50%, 40%, 30%, 25%, 20%, 15%, 10%, 5%, 1%, or fewer of the nucleotides of the nucleic acid-targeting guide RNA participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A.R. Gruber et al., 2008, Cell 106 (1): 23-24; and PA Carr and GM Church, 2009, Nature Biotechnology 27 (12): 1151-62).

In some embodiments, a guide molecule is designed or selected to modulate intermolecular interactions among guide molecules, such as among stem-loop regions of different guide molecules. It will be appreciated that nucleotides within a guide that base-pair to form a stem-loop are also capable of base-pairing to form an intermolecular duplex with a second guide and that such an intermolecular duplex would not have a secondary structure compatible with CRISPR complex formation. Accordingly, it is useful to select or design DR sequences in order to modulate stem-loop formation and CRISPR complex formation. In some embodiments, about or less than about 75%, 50%, 40%, 30%, 25%, 20%, 15%, 10%, 5%, 1%, or fewer of nucleic acid-targeting guides are in intermolecular duplexes. It will be appreciated that stem-loop variation will often be within limits imposed by DR-CRISPR effector interactions. One way to modulate stem-loop formation or change the equilibrium between stem-loop and intermolecular duplex is to vary nucleotide pairs in the stem of the stem-loop of a DR. For example, in one embodiment, a G-C pair is replaced by an A-U or U-A pair. In another embodiment, an A-U pair is substituted for a G-C or a C-G pair. In another embodiment, a naturally occurring nucleotide is replaced by a nucleotide analog. Another way to modulate stem-loop formation or change the equilibrium between stem-loop and intermolecular duplex is to modify the loop of the stem-loop of a DR. Without be bound by theory, the loop can be viewed as an intervening sequence flanked by two sequences that are complementary to each other. When that intervening sequence is not self-complementary, its effect will be to destabilize intermolecular duplex formation. The same principle applies when guides are multiplexed: while the targeting sequences may differ, it may be advantageous to modify the stem-loop region in the DRs of the different guides. Moreover, when guides are multiplexed, the relative activities of the different guides can be modulated by balancing the activity of each individual guide. In certain embodiments, the equilibrium between intermolecular stem-loops vs. intermolecular duplexes is determined. The determination may be made by physical or biochemical means and can be in the presence or absence of a CRISPR effector.

In some embodiments, it is of interest to reduce the susceptibility of the guide molecule to RNA cleavage, such as cleavage by a CRISPR system that cleaves RNA. Accordingly, in particular embodiments, the guide molecule is adjusted to avoid cleavage by a CRISPR system or other RNA-cleaving enzymes.

In certain embodiments, the guide molecule comprises non-naturally occurring nucleic acids and/or non-naturally occurring nucleotides and/or nucleotide analogs, and/or chemically modifications. Preferably, these non-naturally occurring nucleic acids and non-naturally occurring nucleotides are located outside the guide sequence. Non-naturally occurring nucleic acids can include, for example, mixtures of naturally and non-naturally occurring nucleotides. Non-naturally occurring nucleotides and/or nucleotide analogs may be modified at the ribose, phosphate, and/or base moiety. In an embodiment of the invention, a guide nucleic acid comprises ribonucleotides and non-ribonucleotides. In one such embodiment, a guide comprises one or more ribonucleotides and one or more deoxyribonucleotides. In an embodiment of the invention, the guide comprises one or more non-naturally occurring nucleotide or nucleotide analog such as a nucleotide with phosphorothioate linkage, a locked nucleic acid (LNA) nucleotide comprising a methylene bridge between the 2′ and 4′ carbons of the ribose ring, or bridged nucleic acids (BNA). Other examples of modified nucleotides include 2′-O-methyl analogs, 2′-deoxy analogs, or 2′-fluoro analogs. Further examples of modified bases include, but are not limited to, 2-aminopurine, 5-bromo-uridine, pseudouridine, inosine, 7-methylguanosine. Examples of guide RNA chemical modifications include, without limitation, incorporation of 2′-O-methyl (M), 2′-O-methyl 3′ phosphorothioate (MS), S-constrained ethyl (cEt), or 2′-O-methyl 3′thioPACE (MSP) at one or more terminal nucleotides. Such chemically modified guides can comprise increased stability and increased activity as compared to unmodified guides, though on-target vs. off-target specificity is not predictable. (See, Hendel, 2015, Nat Biotechnol. 33 (9): 985-9, doi: 10.1038/nbt.3290, published online 29 Jun. 2015 Ragdarm et al., 0215, PNAS, E7110-E7111; Allerson et al., J. Med. Chem. 2005, 48:901-904; Bramsen et al., Front. Genet., 2012, 3:154; Deng et al., PNAS, 2015, 112:11870-11875; Sharma et al., MedChemComm., 2014, 5:1454-1471; Hendel et al., Nat. Biotechnol. (2015) 33 (9): 985-989; Li et al., Nature Biomedical Engineering, 2017, 1, 0066 DOI: 10.1038/s41551-017-0066). In some embodiments, the 5′ and/or 3′ end of a guide RNA is modified by a variety of functional moieties including fluorescent dyes, polyethylene glycol, cholesterol, proteins, or detection tags. (See Kelly et al., 2016, J. Biotech. 233:74-83). In certain embodiments, a guide comprises ribonucleotides in a region that binds to a target RNA and one or more deoxyribonucleotides and/or nucleotide analogs in a region that binds to a Cas effector. In an embodiment of the invention, deoxyribonucleotides and/or nucleotide analogs are incorporated in engineered guide structures, such as, without limitation, stem-loop regions, and the seed region. In certain embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or 75 nucleotides of a guide is chemically modified. In some embodiments, 3-5 nucleotides at either the 3′ or the 5′ end of a guide is chemically modified. In some embodiments, only minor modifications are introduced in the seed region, such as 2′-F modifications. In some embodiments, 2′-F modification is introduced at the 3′ end of a guide. In certain embodiments, three to five nucleotides at the 5′ and/or the 3′ end of the guide are chemically modified with 2′-O-methyl (M), 2′-O-methyl 3′ phosphorothioate (MS), S-constrained ethyl (cEt), or 2′-O-methyl 3′ thioPACE (MSP). Such modification can enhance genome editing efficiency (see Hendel et al., Nat. Biotechnol. (2015) 33 (9): 985-989). In certain embodiments, all of the phosphodiester bonds of a guide are substituted with phosphorothioates (PS) for enhancing levels of gene disruption. In certain embodiments, more than five nucleotides at the 5′ and/or the 3′ end of the guide are chemically modified with 2′-O-Me, 2′-F or S-constrained ethyl (cEt). Such chemically modified guide can mediate enhanced levels of gene disruption (see Ragdarm et al., 0215, PNAS, E7110-E7111). In an embodiment of the invention, a guide is modified to comprise a chemical moiety at its 3′ and/or 5′ end. Such moieties include, but are not limited to amine, azide, alkyne, thio, dibenzocyclooctyne (DBCO), or Rhodamine, peptides, nuclear localization sequence (NLS), peptide nucleic acid (PNA), polyethylene glycol (PEG), triethylene glycol, or tetraethyleneglycol (TEG). In certain embodiment, the chemical moiety is conjugated to the guide by a linker, such as an alkyl chain. In certain embodiments, the chemical moiety is conjugated to the guide by a linker, such as an alkyl chain. In certain embodiments, the chemical moiety of the modified guide can be used to attach the guide to another molecule, such as DNA, RNA, protein, or nanoparticles. Such chemically modified guide can be used to identify or enrich cells generically edited by a CRISPR system (see Lee et al., eLife, 2017, 6: e25312, DOI: 10.7554).

In some embodiments, 3 nucleotides at each of the 3′ and 5′ ends are chemically modified. In a specific embodiment, the modifications comprise 2′-O-methyl or phosphorothioate analogs. In a specific embodiment, 12 nucleotides in the tetraloop and 16 nucleotides in the stem-loop region are replaced with 2′-O-methyl analogs. Such chemical modifications improve in vivo editing and stability (see Finn et al., Cell Reports (2018), 22:2227-2235). In some embodiments, more than 60 or 70 nucleotides of the guide are chemically modified. In some embodiments, this modification comprises replacement of nucleotides with 2′-O-methyl or 2′-fluoro nucleotide analogs or phosphorothioate (PS) modification of phosphodiester bonds. In some embodiments, the chemical modification comprises 2′-O-methyl or 2′-fluoro modification of guide nucleotides extending outside of the nuclease protein when the CRISPR complex is formed or PS modification of 20 to 30 or more nucleotides of the 3′-terminus of the guide. In a particular embodiment, the chemical modification further comprises 2′-O-methyl analogs at the 5′ end of the guide or 2′-fluoro analogs in the seed and tail regions. Such chemical modifications improve stability to nuclease degradation and maintain or enhance genome-editing activity or efficiency, but modification of all nucleotides may abolish the function of the guide (see Yin et al., Nat. Biotech. (2018), 35 (12): 1179-1187). Such chemical modifications may be guided by knowledge of the structure of the CRISPR complex, including knowledge of the limited number of nuclease and RNA 2′-OH interactions (see Yin et al., Nat. Biotech. (2018), 35 (12): 1179-1187). In some embodiments, one or more guide RNA nucleotides may be replaced with DNA nucleotides. In some embodiments, up to 2, 4, 6, 8, 10, or 12 RNA nucleotides of the 5′-end tail/seed guide region are replaced with DNA nucleotides. In certain embodiments, the majority of guide RNA nucleotides at the 3′ end are replaced with DNA nucleotides. In particular embodiments, 16 guide RNA nucleotides at the 3′ end are replaced with DNA nucleotides. In particular embodiments, 8 guide RNA nucleotides of the 5′-end tail/seed region and 16 RNA nucleotides at the 3′ end are replaced with DNA nucleotides. In particular embodiments, guide RNA nucleotides that extend outside of the nuclease protein when the CRISPR complex is formed are replaced with DNA nucleotides. Such replacement of multiple RNA nucleotides with DNA nucleotides leads to decreased off-target activity but similar on-target activity compared to an unmodified guide; however, replacement of all RNA nucleotides at the 3′ end may abolish the function of the guide (see Yin et al., Nat. Chem. Biol. (2018) 14, 311-316). Such modifications may be guided by knowledge of the structure of the CRISPR complex, including knowledge of the limited number of nuclease and RNA 2′-OH interactions (see Yin et al., Nat. Chem. Biol. (2018) 14, 311-316).

In some embodiments, the guide molecule forms a stemloop with a separate non-covalently linked sequence, which can be DNA or RNA. In particular embodiments, the sequences forming the guide are first synthesized using the standard phosphoramidite synthetic protocol (Herdewijn, P., ed., Methods in Molecular Biology Col 288, Oligonucleotide Synthesis: Methods and Applications, Humana Press, New Jersey (2012)). In some embodiments, these sequences can be functionalized to contain an appropriate functional group for ligation using the standard protocol known in the art (Hermanson, G. T., Bioconjugate Techniques, Academic Press (2013)). Examples of functional groups include, but are not limited to, hydroxyl, amine, carboxylic acid, carboxylic acid halide, carboxylic acid active ester, aldehyde, carbonyl, chlorocarbonyl, imidazolylcarbonyl, hydrozide, semicarbazide, thio semicarbazide, thiol, maleimide, haloalkyl, sulfonyl, ally, propargyl, diene, alkyne, and azide. Once this sequence is functionalized, a covalent chemical bond or linkage can be formed between this sequence and the direct repeat sequence. Examples of chemical bonds include, but are not limited to, those based on carbamates, ethers, esters, amides, imines, amidines, aminotrizines, hydrozone, disulfides, thioethers, thioesters, phosphorothioates, phosphorodithioates, sulfonamides, sulfonates, sulfones, sulfoxides, ureas, thioureas, hydrazide, oxime, triazole, photolabile linkages, C—C bond forming groups such as Diels-Alder cyclo-addition pairs or ring-closing metathesis pairs, and Michael reaction pairs.

In some embodiments, these stem-loop forming sequences can be chemically synthesized. In some embodiments, the chemical synthesis uses automated, solid-phase oligonucleotide synthesis machines with 2′-acetoxyethyl orthoester (2′-ACE) (Scaringe et al., J. Am. Chem. Soc. (1998) 120:11820-11821; Scaringe, Methods Enzymol. (2000) 317:3-18) or 2′-thionocarbamate (2′-TC) chemistry (Dellinger et al., J. Am. Chem. Soc. (2011) 133:11540-11546; Hendel et al., Nat. Biotechnol. (2015) 33:985-989).

In certain embodiments, the guide molecule comprises (1) a guide sequence capable of hybridizing to a target locus and (2) a tracr mate or direct repeat sequence whereby the direct repeat sequence is located upstream (i.e., 5′) or downstream (i.e., 3′) from the guide sequence. In a particular embodiment, the seed sequence (i.e., the sequence essential critical for recognition and/or hybridization to the sequence at the target locus) of the guide sequence is approximately within the first 10 nucleotides of the guide sequence.

In a particular embodiment, the guide molecule comprises a guide sequence linked to a direct repeat sequence, wherein the direct repeat sequence comprises one or more stem loops or optimized secondary structures. In particular embodiments, the direct repeat has a minimum length of 16 nts and a single stem loop. In further embodiments the direct repeat has a length longer than 16 nts, preferably more than 17 nts, and has more than one stem loops or optimized secondary structures. In particular embodiments, the guide molecule comprises or consists of the guide sequence linked to all or part of the natural direct repeat sequence. A CRISPR-cas guide molecule comprises (in 3′ to 5′ direction or in 5′ to 3′ direction): a guide sequence a first complimentary stretch (the “repeat”), a loop (which is typically 4 or 5 nucleotides long), a second complimentary stretch (the “anti-repeat” being complimentary to the repeat), and a poly A (often poly U in RNA) tail (terminator). In certain embodiments, the direct repeat sequence retains its natural architecture and forms a single stem loop. In particular embodiments, certain aspects of the guide architecture can be modified, for example by addition, subtraction, or substitution of features, whereas certain other aspects of guide architecture are maintained. Preferred locations for engineered guide molecule modifications, including but not limited to insertions, deletions, and substitutions include guide termini and regions of the guide molecule that are exposed when complexed with the CRISPR-Cas protein and/or target, for example the stemloop of the direct repeat sequence.

In particular embodiments, the stem comprises at least about 4 bp comprising complementary X and Y sequences, although stems of more, e.g., 5, 6, 7, 8, 9, 10, 11 or 12 or fewer, e.g., 3, 2, base pairs are also contemplated. Thus, for example X2-10 and Y2-10 (wherein X and Y represent any complementary set of nucleotides) may be contemplated. In one aspect, the stem made of the X and Y nucleotides, together with the loop will form a complete hairpin in the overall secondary structure; and this may be advantageous and the amount of base pairs can be any amount that forms a complete hairpin. In one aspect, any complementary X: Y basepairing sequence (e.g., as to length) is tolerated, so long as the secondary structure of the entire guide molecule is preserved. In one aspect, the loop that connects the stem made of X: Y basepairs can be any sequence of the same length (e.g., 4 or 5 nucleotides) or longer that does not interrupt the overall secondary structure of the guide molecule. In one aspect, the stemloop can further comprise, e.g., an MS2 aptamer. In one aspect, the stem comprises about 5-7 bp comprising complementary X and Y sequences, although stems of more or fewer basepairs are also contemplated. In one aspect, non-Watson Crick basepairing is contemplated, where such pairing otherwise generally preserves the architecture of the stemloop at that position.

In particular embodiments, the natural hairpin or stemloop structure of the guide molecule is extended or replaced by an extended stemloop. It has been demonstrated that extension of the stem can enhance the assembly of the guide molecule with the CRISPR-Cas protein (Chen et al. Cell. (2013); 155 (7): 1479-1491). In particular embodiments, the stem of the stemloop is extended by at least 1, 2, 3, 4, 5 or more complementary basepairs (i.e., corresponding to the addition of 2, 4, 6, 8, 10 or more nucleotides in the guide molecule). In particular embodiments, these are located at the end of the stem, adjacent to the loop of the stemloop.

In particular embodiments, the susceptibility of the guide molecule to RNases or to decreased expression can be reduced by slight modifications of the sequence of the guide molecule which do not affect its function. For instance, in particular embodiments, premature termination of transcription, such as premature transcription of U6 Pol-III, can be removed by modifying a putative Pol-III terminator (4 consecutive U's) in the guide molecules sequence. Where such sequence modification is required in the stemloop of the guide molecule, it is preferably ensured by a basepair flip.

In a particular embodiment, the direct repeat may be modified to comprise one or more protein-binding RNA aptamers. In a particular embodiment, one or more aptamers may be included such as part of optimized secondary structure. Such aptamers may be capable of binding a bacteriophage coat protein as detailed further herein.

In some embodiments, the guide molecule forms a duplex with a target RNA comprising at least one target cytosine residue to be edited. Upon hybridization of the guide RNA molecule to the target RNA, the cytidine deaminase binds to the single strand RNA in the duplex made accessible by the mismatch in the guide sequence and catalyzes deamination of one or more target cytosine residues comprised within the stretch of mismatching nucleotides.

A guide sequence, and hence a nucleic acid-targeting guide RNA, may be selected to target any target nucleic acid sequence. The target sequence may be mRNA.

In certain embodiments, the target sequence should be associated with a PAM (protospacer adjacent motif) or PFS (protospacer flanking sequence or site), that is, a short sequence recognized by the CRISPR complex. Depending on the nature of the CRISPR-Cas protein, the target sequence should be selected such that its complementary sequence in the DNA duplex (also referred to herein as the non-target sequence) is upstream or downstream of the PAM.

Further, engineering of the PAM Interacting (PI) domain may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523 (7561): 481-5. doi: 10.1038/nature14592.

In particular embodiments, the guide is an escorted guide. By “escorted” is meant that the CRISPR-Cas system or complex or guide is delivered to a selected time or place within a cell, so that activity of the CRISPR-Cas system or complex or guide is spatially or temporally controlled. For example, the activity and destination of the 3 CRISPR-Cas system or complex or guide may be controlled by an escort RNA aptamer sequence that has binding affinity for an aptamer ligand, such as a cell surface protein or other localized cellular component. Alternatively, the escort aptamer may for example be responsive to an aptamer effector on or in the cell, such as a transient effector, such as an external energy source that is applied to the cell at a particular time.

The escorted CRISPR-Cas systems or complexes have a guide molecule with a functional structure designed to improve guide molecule structure, architecture, stability, genetic expression, or any combination thereof. Such a structure can include an aptamer.

Aptamers are biomolecules that can be designed or selected to bind tightly to other ligands, for example using a technique called systematic evolution of ligands by exponential enrichment (SELEX; Tuerk C, Gold L: “Systematic evolution of ligands by exponential enrichment: RNA ligands to bacteriophage T4 DNA polymerase.” Science 1990, 249:505-510). Nucleic acid aptamers can for example be selected from pools of random-sequence oligonucleotides, with high binding affinities and specificities for a wide range of biomedically relevant targets, suggesting a wide range of therapeutic utilities for aptamers (Keefe, Anthony D., Supriya Pai, and Andrew Ellington. “Aptamers as therapeutics.” Nature Reviews Drug Discovery 9.7 (2010): 537-550). These characteristics also suggest a wide range of uses for aptamers as drug delivery vehicles (Levy-Nissenbaum, Etgar, et al. “Nanotechnology and aptamers: applications in drug delivery.” Trends in biotechnology 26.8 (2008): 442-449; and, Hicke B J, Stephens A W. “Escort aptamers: a delivery service for diagnosis and therapy.” J Clin Invest 2000, 106:923-928.). Aptamers may also be constructed that function as molecular switches, responding to a que by changing properties, such as RNA aptamers that bind fluorophores to mimic the activity of green fluorescent protein (Paige, Jeremy S., Karen Y. Wu, and Samie R. Jaffrey. “RNA mimics of green fluorescent protein.” Science 333.6042 (2011): 642-646). It has also been suggested that aptamers may be used as components of targeted siRNA therapeutic delivery systems, for example targeting cell surface proteins (Zhou, Jiehua, and John J. Rossi. “Aptamer-targeted cell-specific RNA interference.” Silence 1.1 (2010): 4).

Accordingly, in particular embodiments, the guide molecule is modified, e.g., by one or more aptamer(s) designed to improve guide molecule delivery, including delivery across the cellular membrane, to intracellular compartments, or into the nucleus. Such a structure can include, either in addition to the one or more aptamer(s) or without such one or more aptamer(s), moiety(ies) so as to render the guide molecule deliverable, inducible or responsive to a selected effector. The invention accordingly comprehends a guide molecule that responds to normal or pathological physiological conditions, including without limitation pH, hypoxia, 02 concentration, temperature, protein concentration, enzymatic concentration, lipid structure, light exposure, mechanical disruption (e.g., ultrasound waves), magnetic fields, electric fields, or electromagnetic radiation.

Light responsiveness of an inducible system may be achieved via the activation and binding of cryptochrome-2 and CIB1. Blue light stimulation induces an activating conformational change in cryptochrome-2, resulting in recruitment of its binding partner CIB1. This binding is fast and reversible, achieving saturation in <15 sec following pulsed stimulation and returning to baseline <15 min after the end of stimulation. These rapid binding kinetics result in a system temporally bound only by the speed of transcription/translation and transcript/protein degradation, rather than uptake and clearance of inducing agents. Crytochrome-2 activation is also highly sensitive, allowing for the use of low light intensity stimulation and mitigating the risks of phototoxicity. Further, in a context such as the intact mammalian brain, variable light intensity may be used to control the size of a stimulated region, allowing for greater precision than vector delivery alone may offer.

The invention contemplates energy sources such as electromagnetic radiation, sound energy or thermal energy to induce the guide. Advantageously, the electromagnetic radiation is a component of visible light. In a preferred embodiment, the light is a blue light with a wavelength of about 450 to about 495 nm. In an especially preferred embodiment, the wavelength is about 488 nm. In another preferred embodiment, the light stimulation is via pulses. The light power may range from about 0-9 mW/cm2. In a preferred embodiment, a stimulation paradigm of as low as 0.25 sec every 15 sec should result in maximal activation.

The chemical or energy sensitive guide may undergo a conformational change upon induction by the binding of a chemical source or by the energy allowing it act as a guide and have the CRISPR-Cas system or complex function. The invention can involve applying the chemical source or energy so as to have the guide function and the CRISPR-Cas system or complex function; and optionally further determining that the expression of the genomic locus is altered.

There are several different designs of this chemical inducible system: 1. ABI-PYL based system inducible by Abscisic Acid (ABA) (see, e.g., stke.sciencemag.org/cgi/content/abstract/sigtrans; 4/164/rs2), 2. FKBP-FRB based system inducible by rapamycin (or related chemicals based on rapamycin) (see, e.g., www.nature.com/nmeth/journal/v2/n6/full/nmeth763.htmD, 3. GID1-GAI based system inducible by Gibberellin (GA) (see, e.g., www.nature.com/nchembio/journal/v8/n5/full/nchembio. 922.htmD.

A chemical inducible system can be an estrogen receptor (ER) based system inducible by 4-hydroxytamoxifen (4OHT) (see, e.g., www.pnas.org/content/1Apr. 3, 1027.abstract). A mutated ligand-binding domain of the estrogen receptor called ERT2 translocates into the nucleus of cells upon binding of 4-hydroxytamoxifen. In further embodiments of the invention any naturally occurring or engineered derivative of any nuclear receptor, thyroid hormone receptor, retinoic acid receptor, estrogen receptor, estrogen-related receptor, glucocorticoid receptor, progesterone receptor, androgen receptor may be used in inducible systems analogous to the ER based inducible system.

Another inducible system is based on the design using Transient receptor potential (TRP) ion channel based system inducible by energy, heat or radio-wave (see, e.g., www.sciencemag.org/content/336/6081/604). These TRP family proteins respond to different stimuli, including light and heat. When this protein is activated by light or heat, the ion channel will open and allow the entering of ions such as calcium into the plasma membrane. This influx of ions will bind to intracellular ion interacting partners linked to a polypeptide including the guide and the other components of the CRISPR-Cas complex or system, and the binding will induce the change of sub-cellular localization of the polypeptide, leading to the entire polypeptide entering the nucleus of cells. Once inside the nucleus, the guide protein and the other components of the CRISPR-Cas complex will be active and modulating target gene expression in cells.

While light activation may be an advantageous embodiment, sometimes it may be disadvantageous especially for in vivo applications in which the light may not penetrate the skin or other organs. In this instance, other methods of energy activation are contemplated, in particular, electric field energy and/or ultrasound which have a similar effect.

Electric field energy is preferably administered substantially as described in the art, using one or more electric pulses of from about 1 Volt/cm to about 10 kVolts/cm under in vivo conditions. Instead of or in addition to the pulses, the electric field may be delivered in a continuous manner. The electric pulse may be applied for between 1 us and 500 milliseconds, preferably between 1 us and 100 milliseconds. The electric field may be applied continuously or in a pulsed manner for 5 about minutes.

As used herein, ‘electric field energy’ is the electrical energy to which a cell is exposed. Preferably, the electric field has a strength of from about 1 Volt/cm to about 10 k Volts/cm or more under in vivo conditions (see WO97/49450).

As used herein, the term “electric field” includes one or more pulses at variable capacitance and voltage and including exponential and/or square wave and/or modulated wave and/or modulated square wave forms. References to electric fields and electricity should be taken to include reference the presence of an electric potential difference in the environment of a cell. Such an environment may be set up by way of static electricity, alternating current (AC), direct current (DC), etc., as known in the art. The electric field may be uniform, non-uniform or otherwise, and may vary in strength and/or direction in a time dependent manner.

Single or multiple applications of electric field, as well as single or multiple applications of ultrasound are also possible, in any order and in any combination. The ultrasound and/or the electric field may be delivered as single or multiple continuous applications, or as pulses (pulsatile delivery).

Electroporation has been used in both in vitro and in vivo procedures to introduce foreign material into living cells. With in vitro applications, a sample of live cells is first mixed with the agent of interest and placed between electrodes such as parallel plates. Then, the electrodes apply an electrical field to the cell/implant mixture. Examples of systems that perform in vitro electroporation include the Electro Cell Manipulator ECM600 product, and the Electro Square Porator T820, both made by the BTX Division of Genetronics, Inc (see U.S. Pat. No. 5,869,326).

The known electroporation techniques (both in vitro and in vivo) function by applying a brief high voltage pulse to electrodes positioned around the treatment region. The electric field generated between the electrodes causes the cell membranes to temporarily become porous, whereupon molecules of the agent of interest enter the cells. In known electroporation applications, this electric field comprises a single square wave pulse on the order of 1000 V/cm, of about 100. mu.s duration. Such a pulse may be generated, for example, in known applications of the Electro Square Porator T820.

Preferably, the electric field has a strength of from about 1 V/cm to about 10 kV/cm under in vitro conditions. Thus, the electric field may have a strength of 1 V/cm, 2 V/cm, 3 V/cm, 4 V/cm, 5 V/cm, 6 V/cm, 7 V/cm, 8 V/cm, 9 V/cm, 10 V/cm, 20 V/cm, 50 V/cm, 100 V/cm, 200 V/cm, 300 V/cm, 400 V/cm, 500 V/cm, 600 V/cm, 700 V/cm, 800 V/cm, 900 V/cm, 1 kV/cm, 2 kV/cm, 5 kV/cm, 10 kV/cm, 20 kV/cm, 50 kV/cm or more. More preferably from about 0.5 kV/cm to about 4.0 kV/cm under in vitro conditions. Preferably the electric field has a strength of from about 1 V/cm to about 10 kV/cm under in vivo conditions. However, the electric field strengths may be lowered where the number of pulses delivered to the target site are increased. Thus, pulsatile delivery of electric fields at lower field strengths is envisaged.

Preferably the application of the electric field is in the form of multiple pulses such as double pulses of the same strength and capacitance or sequential pulses of varying strength and/or capacitance. As used herein, the term “pulse” includes one or more electric pulses at variable capacitance and voltage and including exponential and/or square wave and/or modulated wave/square wave forms.

Preferably the electric pulse is delivered as a waveform selected from an exponential wave form, a square wave form, a modulated wave form and a modulated square wave form.

A preferred embodiment employs direct current at low voltage. Thus, Applicants disclose the use of an electric field which is applied to the cell, tissue or tissue mass at a field strength of between 1V/cm and 20V/cm, for a period of 100 milliseconds or more, preferably 15 minutes or more.

Ultrasound is advantageously administered at a power level of from about 0.05 W/cm2 to about 100 W/cm². Diagnostic or therapeutic ultrasound may be used, or combinations thereof.

As used herein, the term “ultrasound” refers to a form of energy which consists of mechanical vibrations the frequencies of which are so high they are above the range of human hearing. Lower frequency limit of the ultrasonic spectrum may generally be taken as about 20 KHz. Most diagnostic applications of ultrasound employ frequencies in the range 1 and 15 MHz′ (From Ultrasonics in Clinical Diagnosis, P. N. T. Wells, ed., 2nd. Edition, Publ. Churchill Livingstone [Edinburgh, London & NY, 1977]).

Ultrasound has been used in both diagnostic and therapeutic applications. When used as a diagnostic tool (“diagnostic ultrasound”), ultrasound is typically used in an energy density range of up to about 100 mW/cm2 (FDA recommendation), although energy densities of up to 750 mW/cm2 have been used. In physiotherapy, ultrasound is typically used as an energy source in a range up to about 3 to 4 W/cm2 (WHO recommendation). In other therapeutic applications, higher intensities of ultrasound may be employed, for example, HIFU at 100 W/cm up to 1 kW/cm2 (or even higher) for short periods of time. The term “ultrasound” as used in this specification is intended to encompass diagnostic, therapeutic and focused ultrasound.

Focused ultrasound (FUS) allows thermal energy to be delivered without an invasive probe (see Morocz et al 1998 Journal of Magnetic Resonance Imaging Vol. 8, No. 1, pp. 136-142. Another form of focused ultrasound is high intensity focused ultrasound (HIFU) which is reviewed by Moussatov et al in Ultrasonics (1998) Vol. 36, No. 8, pp. 893-900 and TranHuuHue et al in Acustica (1997) Vol. 83, No. 6, pp. 1103-1106.

Preferably, a combination of diagnostic ultrasound and a therapeutic ultrasound is employed. This combination is not intended to be limiting, however, and the skilled reader will appreciate that any variety of combinations of ultrasound may be used. Additionally, the energy density, frequency of ultrasound, and period of exposure may be varied.

Preferably the exposure to an ultrasound energy source is at a power density of from about 0.05 to about 100 Wcm−2. Even more preferably, the exposure to an ultrasound energy source is at a power density of from about 1 to about 15 Wcm−2.

Preferably the exposure to an ultrasound energy source is at a frequency of from about 0.015 to about 10.0 MHz. More preferably the exposure to an ultrasound energy source is at a frequency of from about 0.02 to about 5.0 MHz or about 6.0 MHz. Most preferably, the ultrasound is applied at a frequency of 3 MHz.

Preferably the exposure is for periods of from about 10 milliseconds to about 60 minutes. Preferably the exposure is for periods of from about 1 second to about 5 minutes. More preferably, the ultrasound is applied for about 2 minutes. Depending on the particular target cell to be disrupted, however, the exposure may be for a longer duration, for example, for 15 minutes.

Advantageously, the target tissue is exposed to an ultrasound energy source at an acoustic power density of from about 0.05 Wcm−2 to about 10 Wcm−2 with a frequency ranging from about 0.015 to about 10 MHz (see WO 98/52609). However, alternatives are also possible, for example, exposure to an ultrasound energy source at an acoustic power density of above 100 Wcm−2, but for reduced periods of time, for example, 1000 Wcm−2 for periods in the millisecond range or less.

Preferably the application of the ultrasound is in the form of multiple pulses; thus, both continuous wave and pulsed wave (pulsatile delivery of ultrasound) may be employed in any combination. For example, continuous wave ultrasound may be applied, followed by pulsed wave ultrasound, or vice versa. This may be repeated any number of times, in any order and combination. The pulsed wave ultrasound may be applied against a background of continuous wave ultrasound, and any number of pulses may be used in any number of groups.

Preferably, the ultrasound may comprise pulsed wave ultrasound. In a highly preferred embodiment, the ultrasound is applied at a power density of 0.7 Wcm−2 or 1.25 Wcm−2 as a continuous wave. Higher power densities may be employed if pulsed wave ultrasound is used.

Use of ultrasound is advantageous as, like light, it may be focused accurately on a target. Moreover, ultrasound is advantageous as it may be focused more deeply into tissues unlike light. It is therefore better suited to whole-tissue penetration (such as but not limited to a lobe of the liver) or whole organ (such as but not limited to the entire liver or an entire muscle, such as the heart) therapy. Another important advantage is that ultrasound is a non-invasive stimulus which is used in a wide variety of diagnostic and therapeutic applications. By way of example, ultrasound is well known in medical imaging techniques and, additionally, in orthopedic therapy. Furthermore, instruments suitable for the application of ultrasound to a subject vertebrate are widely available and their use is well known in the art.

In particular embodiments, the guide molecule is modified by a secondary structure to increase the specificity of the CRISPR-Cas system and the secondary structure can protect against exonuclease activity and allow for 5′ additions to the guide sequence also referred to herein as a protected guide molecule.

In one aspect, the invention provides for hybridizing a “protector RNA” to a sequence of the guide molecule, wherein the “protector RNA” is an RNA strand complementary to the 3′ end of the guide molecule to thereby generate a partially double-stranded guide RNA. In an embodiment of the invention, protecting mismatched bases (i.e. the bases of the guide molecule which do not form part of the guide sequence) with a perfectly complementary protector sequence decreases the likelihood of target RNA binding to the mismatched basepairs at the 3′ end. In particular embodiments of the invention, additional sequences comprising an extended length may also be present within the guide molecule such that the guide comprises a protector sequence within the guide molecule. This “protector sequence” ensures that the guide molecule comprises a “protected sequence” in addition to an “exposed sequence” (comprising the part of the guide sequence hybridizing to the target sequence). In particular embodiments, the guide molecule is modified by the presence of the protector guide to comprise a secondary structure such as a hairpin. Advantageously there are three or four to thirty or more, e.g., about 10 or more, contiguous base pairs having complementarity to the protected sequence, the guide sequence or both. It is advantageous that the protected portion does not impede thermodynamics of the CRISPR-Cas system interacting with its target. By providing such an extension including a partially double stranded guide molecule, the guide molecule is considered protected and results in improved specific binding of the CRISPR-Cas complex, while maintaining specific activity.

In particular embodiments, use is made of a truncated guide (tru-guide), i.e. a guide molecule which comprises a guide sequence which is truncated in length with respect to the canonical guide sequence length. As described by Nowak et al. (Nucleic Acids Res (2016) 44 (20): 9555-9564), such guides may allow catalytically active CRISPR-Cas enzyme to bind its target without cleaving the target RNA. In particular embodiments, a truncated guide is used which allows the binding of the target but retains only nickase activity of the CRISPR-Cas enzyme.

### Tn7 and Tn7-Like Transposases

The transposases in the systems herein may be CRISPR-associated transposases (also used interchangeably with “Cas-associated transposases,” “CRISPR-associated Tn7 transposases,” and “CRISPR-associated transposase proteins” herein) or functional fragments thereof. CRISPR-associated transposases may include any transposase that can be directed to or recruited to a region of a target polynucleotide by sequence-specific binding of a CRISPR-Cas complex. CRISPR-associated transposases may include any transposases that associate (e.g., form a complex) with one or more components in a CRISPR-Cas system, e.g., Cas protein, guide molecule etc.). In certain example embodiments, CRISPR-associated transposases may be fused or tethered (e.g. by a linker) to one or more components in a CRISPR-Cas system, e.g., Cas protein, guide molecule etc.).

The term “transposon,” as used herein, refers to a polynucleotide (or nucleic acid segment), which may be recognized by a transposase or an integrase enzyme and which is a component of a functional nucleic acid-protein complex (e.g., a transpososome, or transposon complex) capable of transposition. Transposons employ a variety of regulatory mechanisms to maintain transposition at a low frequency and sometimes coordinate transposition with various cell processes. Some prokaryotic transposons can also mobilize functions that benefit the host or otherwise help maintain the element.

The term “transposase” (used interchangeably herein with “transposition protein”) refers to an enzyme, which is a component of a functional nucleic acid-protein complex capable of transposition and which mediates transposition. The transposase may comprise a single protein or comprise multiple protein sub-units. A transposase may be an enzyme capable of forming a functional complex with a transposon end or transposon end sequences. The term “transposase” may also refer in certain embodiments to integrases. The expression “transposition reaction” used herein refers to a reaction wherein a transposase inserts a donor polynucleotide sequence in or adjacent to an insertion site on a target polynucleotide. The insertion site may contain a sequence or secondary structure recognized by the transposase and/or an insertion motif sequence where the transposase cuts or creates staggered breaks in the target polynucleotide into which the donor polynucleotide sequence may be inserted. Exemplary components in a transposition reaction include a transposon, comprising the donor polynucleotide sequence to be inserted, and a transposase or an integrase enzyme. The term “transposon end sequence” as used herein refers to the nucleotide sequences at the distal ends of a transposon. The transposon end sequences may be responsible for identifying the donor polynucleotide for transposition. The transposon end sequences may be the DNA sequences the transpose enzyme uses in order to form transpososome complex and to perform a transposition reaction.

### Tn7 Transposases

In one example embodiment, the CAST system comprises one or more Tn7 transposases or Tn7-like transposases. As used herein, the term “Tn7 transposase” refers to the canonical transposases encoded by the bacterial Tn7 transposon. These Tn7 transposases comprise the transposases TnsA, TnsB, TnsC, TnsD, and TnsE. The term “Tn7-like transposase” refers to transposases encoded by non-Tn7 transposons (i.e., “Tn7-like transposons”). These Tn7-like transposases include canonical Tn7 transposases (e.g., TnsA, TnsB, TnsC, etc.) as well as homologs of Tn7 transposases (e.g., proteins sharing at least 50%, at least 60%, at least 70%, at least 80%, at least 90%, at least 95%, or at least 99% sequence identity with a Tn7 transposase).

Transposons employ a variety of regulatory mechanisms to maintain transposition at a low frequency and sometimes coordinate transposition with various cell processes. Some prokaryotic transposons can also mobilize functions that benefit the host or otherwise help maintain the element. Certain transposons have evolved mechanisms of tight control over target site selection, the most notable example being the Tn7 family (see Peters J E (2014) Tn7. Microbiol Spectr 2:1-20). Three transposon-encoded proteins form the core transposition machinery of Tn7: a heteromeric transposase consisting of TnsA and TnsB; and a regulator protein, TnsC. In addition to the core TnsABC transposition proteins, Tn7 elements encode dedicated target site selection proteins, TnsD and TnsE. In conjunction with TnsABC, the sequence-specific DNA-binding protein TnsD directs transposition into a conserved site referred to as the “Tn7 attachment site,” attTn7. TnsD is a member of a large family of proteins that also includes TniQ, a protein found in other types of bacterial transposons. TniQ has been shown to target transposition into resolution sites of plasmids.

In example embodiments, the engineered CAST system comprises TnsA, TnsB, and TnsC. In some embodiments, TnsA and TnsB may comprise a fusion protein (denoted herein as “TnsAB”) to advantageously reduce the size and complexity of the system (i.e., to reduce the number of components and promote efficient packaging and delivery of components). Thus, in some embodiments, the engineered CAST system comprises TnsAB and TnsC. In some embodiments, the engineered CAST system further comprises TniQ. In some embodiments, the engineered CAST system further comprises TniQ and TnsF. Although TniQ may serve as an adaptor protein, bridging TnsF and the TnsABC transposition proteins, its presence in the engineered CAST system is not required for transposition. This, like the presence of TnsAB, also advantageously reduces the size and complexity of the system. Thus, in an example embodiment, the engineered CAST system may comprise TnsA, TnsB, TnsC, and TnsF. In another embodiment, the engineered CAST system may comprise TnsAB, TnsC, and TnsF.

As used herein, a right end sequence element or a left end sequence element are made in reference to an example Tn7 transposon. The general structure of the left end (LE) and right end (RE) sequence elements of canonical Tn7 is established. Tn7 ends comprise a series of 22-bp TnsB-binding sites. Flanking the most distal TnsB-binding sites is an 8-bp terminal sequence ending with 5′-TGT-3′/3′-ACA-5′. The right end of Tn7 contains four overlapping TnsB-binding sites in the ˜90-bp right end element. The left end contains three TnsB-binding sites dispersed in the ˜150-bp left end of the element. The number and distribution of TnsB-binding sites can vary among Tn7-like elements. End sequences of Tn7-related elements can be determined by identifying the directly repeated 5-bp target site duplication, the terminal 8-bp sequence, and 22-bp TnsB-binding sites (Peters J E et al., 2017). Example Tn7 elements, including right end sequence element and left end sequence element include those described in Parks A R, Plasmid, 2009 January; 61 (1): 1-14.

### Donor Polynucleotides

The systems may comprise one or more donor polynucleotides (e.g., for insertion into the target polynucleotide). A donor polynucleotide may be an equivalent of a transposable element that can be inserted or integrated to a target site. For example, the donor polynucleotide may comprise a polynucleotide to be inserted, a left element sequence, and a right element sequence. The donor polynucleotide may be or comprise one or more components of a transposon. A donor polynucleotide may be any type of polynucleotides, including, but not limited to, a gene, a gene fragment, a non-coding polynucleotide, a regulatory polynucleotide, a synthetic polynucleotide, etc.

In some embodiments, the donor polynucleotide is linear. In some embodiments, the donor polynucleotide is circular. In some examples, the donor polynucleotide has a single strand break (a nick). In some cases, the single strand break is on or close to the 3′ end of the donor polynucleotide. In some cases, the single strand break is on or close to the 5′ end of the donor polynucleotide.

The donor polynucleotides may be inserted to the upstream or downstream of a PAM sequence of a target polynucleotide. For CRISPR-associated transposases, the donor polynucleotide may be inserted at a position between 10 bases and 200 bases, e.g., between 20 bases and 150 bases, between 30 bases and 100 bases, between 45 bases and 70 bases, between 45 bases and 60 bases, between 55 bases and 70 bases, between 49 bases and 56 bases or between 60 bases and 66 bases, from a PAM sequence on the target polynucleotide. In some cases, the insertion is at a position upstream of the PAM sequence. In some cases, the insertion is at a position downstream of the PAM sequence. In some cases, the insertion is at a position from 49 to 56 bases or base pairs downstream from a PAM sequence. In some cases, the insertion is at a position from 60 to 66 bases or base pairs downstream from a PAM sequence.

The donor polynucleotide may be used for editing the target polynucleotide. In some cases, the donor polynucleotide comprises one or more mutations to be introduced into the target polynucleotide. Examples of such mutations include substitutions, deletions, insertions, or a combination thereof. The mutations may cause a shift in an open reading frame on the target polynucleotide. In some cases, the donor polynucleotide alters a stop codon in the target polynucleotide. For example, the donor polynucleotide may correct a premature stop codon. The correction may be achieved by deleting the stop codon or introduces one or more mutations to the stop codon. In other example embodiments, the donor polynucleotide addresses loss of function mutations, deletions, or translocations that may occur, for example, in certain disease contexts by inserting or restoring a functional copy of a gene, or functional fragment thereof, or a functional regulatory sequence or functional fragment of a regulatory sequence. A functional fragment refers to less than the entire copy of a gene by providing sufficient nucleotide sequence to restore the functionality of a wild type gene or non-coding regulatory sequence (e.g., sequences encoding long non-coding RNA). In certain example embodiments, the systems disclosed herein may be used to replace a single allele of a defective gene or defective fragment thereof. In another example embodiment, the systems disclosed herein may be used to replace both alleles of a defective gene or defective gene fragment. A “defective gene” or “defective gene fragment” is a gene or portion of a gene that when expressed fails to generate a functioning protein or non-coding RNA with functionality of a corresponding wild-type gene. In certain example embodiments, these defective genes may be associated with one or more disease phenotypes. In certain example embodiments, the defective gene or gene fragment is not replaced but the systems described herein are used to insert donor polynucleotides that encode gene or gene fragments that compensate for or override defective gene expression such that cell phenotypes associated with defective gene expression are eliminated or changed to a different or desired cellular phenotype.

In certain embodiments of the invention, the donor may include, but not be limited to, genes or gene fragments, encoding proteins or RNA transcripts to be expressed, regulatory elements, repair templates, and the like. According to the invention, the donor polynucleotides may comprise left end and right end sequence elements that function with transposition components that mediate insertion.

In certain cases, the donor polynucleotide manipulates a splicing site on the target polynucleotide. In some examples, the donor polynucleotide disrupts a splicing site. The disruption may be achieved by inserting the polynucleotide to a splicing site and/or introducing one or more mutations to the splicing site. In certain examples, the donor polynucleotide may restore a splicing site. For example, the polynucleotide may comprise a splicing site sequence.

The donor polynucleotide to be inserted may has a size from 10 bases to 50 kb in length, e.g., from 50 to 40 kb, from 100 and 30 kb, from 100 bases to 300 bases, from 200 bases to 400 bases, from 300 bases to 500 bases, from 400 bases to 600 bases, from 500 bases to 700 bases, from 600 bases to 800 bases, from 700 bases to 900 bases, from 800 bases to 1000 bases, from 900 bases to from 1100 bases, from 1000 bases to 1200 bases, from 1100 bases to 1300 bases, from 1200 bases to 1400 bases, from 1300 bases to 1500 bases, from 1400 bases to 1600 bases, from 1500 bases to 1700 bases, from 600 bases to 1800 bases, from 1700 bases to 1900 bases, from 1800 bases to 2000 bases, from 1900 bases to 2100 bases, from 2000 bases to 2200 bases, from 2100 bases to 2300 bases, from 2200 bases to 2400 bases, from 2300 bases to 2500 bases, from 2400 bases to 2600 bases, from 2500 bases to 2700 bases, from 2600 bases to 2800 bases, from 2700 bases to 2900 bases, or from 2800 bases to 3000 bases in length.

Tn7 Systems with Novel Tnsf Target Site Selection Protein

In some embodiments, the engineered systems may comprise one or more Tn7 transposases comprising a novel target selector referred to herein as TnsF. TnsF is a previously uncharacterized target site selection protein found in the Tn6022 family of Tn7-like transposons. As described in further detail herein, TnsF is characterized by a novel domain architecture exhibiting significant structural similarity to the tyrosine recombinase superfamily member XerH. However, TnsF lacks the catalytic activity of tyrosine recombinases. TnsF has an N-terminal region containing multiple zinc fingers; interior tandem DNA-binding domains, CB1 and CB2, positioned between the N- and C-termini; and a C-terminal, DNA-binding partial catalytic (pCAT) domain. The pCAT domain lacks the tyrosine recombinase catalytic site normally found in the full-length CAT domain of tyrosine recombinases. Within the context of Tn6022, TnsF helps mediate site-specific insertion of the transposon into the comM gene, which encodes a Mg chelatase. The TnsF attachment site within the comM gene spans a region of 50 base pairs upstream and 50 base pairs downstream of the transposon insertion site.

In certain example embodiments, the engineered system comprises TnsA, TnsB, TnsC, and TnsF. In other embodiments, the engineered system comprises TnsAB, TnsC, and TnsF. In other embodiments, the engineered system comprises TnsA, TnsB, TnsC, TniQ, and TnsF. In other embodiments, the engineered system comprises TnsAB, TnsC, TniQ, and TnsF.

TnsF is a modular protein that may be incorporated in Tn7 systems as well as CAST 1-D systems, and the nature of its structural domain architecture may provide pathways towards engineering its biomolecular function. In certain example embodiments, TnsF may be engineered to alter target site selection. In some embodiments, target site selection may be altered by mutating one or more of the DNA-binding domains. In some embodiments, target site selection may be altered by mutating CB1. In some embodiments, target site selection may be altered by mutating CB2. In some embodiments, target site selection may be altered by mutating CB1 and CB2. In some embodiments, target site selection may be altered by mutating the pCAT domain. It will be understood by a person skilled in the art that the target site selection activity of TnsF may be altered by any of the various means known in the art to engineer protein activity. In addition to conventional molecular biology techniques, TnsF may be engineered using phage-assisted continuous directed evolution (PAGE) (Esvelt et al., Nature 472, 499-503 (2011)).

In certain example embodiments, the engineered Tn7 system comprising TnsF may be used in combination with one or more gene editing systems. In such an embodiment, the gene editing system first inserts the TnsF attachment/transposon insertion site into the recipient polynucleotide, and the engineered Tn7 system then mediates transposition of a donor polynucleotide at the transposon insertion site.

For example, a Type II or Type V CRISPR-Cas system may be used to insert the TnsF recognition site in a target polynucleotide, e.g., via HDR, and the Tn7-TnsF system then facilitated transposition of a donor sequence at the marked recognitions sites. The Tn7-TnsF system may also be used with prime editing systems using a similar concept, i.e., the prime editing system inserts the TnsF recognition site and the Tn7 transposase facilitates insertion of a donor polynucleotide at the marked insertion site. Examples of such prime editing systems and methods are further described in Anzalone A V et al., Search-and-replace genome editing without double-strand breaks or donor DNA, Nature. 2019 Oct. 21. doi: 10.1038/s41586-019-1711-4, which is incorporated by reference herein in its entirety. Following integration of the TnsF attachment/transposon insertion site at the recipient polynucleotide, TnsF may bind to the TnsF attachment site and help mediate transposition by the TnsABC proteins.

### TSY Systems

The present disclosure also provides an engineered system comprising one or more components of a non-Tn7 transposon system featuring a catalytically active TnsF-like system termed Tsy (Target Selector based on tyrosine (Y) recombinase). This system lacks Tn7 components, but instead includes genes encoding a tyrosine recombinase (YRec), a small helix-turn-helix (HTH) domain protein, and a TnsF homolog having a predicted catalytic nuclease domain. In some cases, the transposon system includes a gene encoding a GIY-YIG nuclease. The TnsF homolog of the Tsy system has a similar domain architecture to the TnsF found in the Tn7 transposases described above, but includes a predicted active nuclease. The TnsF homolog has an N-terminal region containing multiple zinc fingers; interior tandem DNA-binding domains, CB1 and CB2, positioned between the N- and C-termini; and a C-terminal, DNA-binding catalytic (CAT) domain. In contrast to the pCAT domain of the Tn7 TnsF, the CAT domain of Tsy TnsF comprises the tyrosine recombinase catalytic site. Tsy helps mediate site-specific insertion into the comM gene at an insertion site that is about 67 base pairs downstream of the Tn6022 insertion site. The Tsy attachment site is 40 base pairs upstream of the Tsy transposon insertion site.

In certain example embodiments, Tsy-TnsF may be engineered to alter target site selection. In some embodiments, target site selection may be altered by mutating one or more of the DNA-binding domains. In some embodiments, target site selection may be altered by mutating CB1. In some embodiments, target site selection may be altered by mutating CB2. In some embodiments, target site selection may be altered by mutating CB1 and CB2. In some embodiments, target site selection may be altered by mutating the CAT domain. It will be understood by a person skilled in the art that the target site selection activity of Tsy-TnsF may be altered by any of the various means known in the art to engineer protein activity. In addition to conventional molecular biology techniques, Tsy-TnsF may be engineered using phage-assisted continuous directed evolution (PAGE) (Esvelt et al., Nature 472, 499-503 (2011)).

In certain example embodiments, the engineered system comprises one or more tyrosine recombinases, one or more helix-turn-helix (HTH) domain proteins, and one or more TnsF homologs comprising a catalytic nuclease domain. In some embodiments, the engineered system further comprises one or more GIY-YIG nucleases.

In certain example embodiments, the engineered non-Tn7 system comprising the Tsy-TnsF may be used in combination with one or more gene editing systems. In such an embodiment, the gene editing system first inserts the Tsy-TnsF attachment/transposon insertion site into the recipient polynucleotide, and the engineered non-Tn7 system then mediates transposition of a donor polynucleotide at the transposon insertion site.

For example, a Type II or Type V CRISPR-Cas system may be used to insert the TnsF recognition site in a target polynucleotide, e.g., via HDR, and the Tn7-TnsF system then facilitated transposition of a donor sequence at the marked recognitions sites. The Tn7-TnsF system may also be used with prime editing systems using a similar concept, i.e., the prime editing system inserts the TnsF recognition site and the Tn7 transposase facilitates insertion of a donor polynucleotide at the marked insertion site. Examples of such prime editing systems and methods are further described in Anzalone A V et al., Search-and-replace genome editing without double-strand breaks or donor DNA, Nature. 2019 Oct. 21. doi: 10.1038/s41586-019-1711-4, which is incorporated by reference herein in its entirety. Following integration of the TnsF attachment/transposon insertion site at the recipient polynucleotide, TnsF may bind to the TnsF attachment site and help mediate transposition by the TnsABC proteins.

### Protein Modifications

The polypeptide components of the above described gene editing systems, Tn7-CAST-ID, Tn7-like transposases comprising a TnsF target selection, and non-Tn7-lik transposoase comprising a Tsy-TnsF target selector, may comprise one or more modifications to one or more components of each system, where the components of the CRISPR-Cas Type I-D protein complex or the components of the Tn7-like or non-Tn7-like protein complexes. As used herein, the term “modified” with regard to a Fanzor polypeptide generally refers to a Fanzor polypeptide having one or more modifications or mutations (including point mutations, truncations, insertions, deletions, chimeras, fusion proteins, etc.) compared to the wild-type counterpart from which it is derived. By derived is meant that the derived polypeptide is largely based, in the sense of having a high degree of sequence homology with, a wildtype protein, but that it has been mutated (modified) in some way as known in the art or as described herein.

The Cas 1-D polypeptide, TnsF target selector, or TsY-TnsF target selectors may be catalytically inactive (also referred as dead). As used herein, a catalytically inactive or dead polypeptide may have reduced, or no enzymatic activity compared to a wildtype counterpart enzyme. In some cases, a catalytically inactive or dead nuclease may have nickase activity. Such a catalytically inactive or dead enzyme may not make either double-strand or single-strand break or facilitate recombination or insertion on a target polynucleotide but may still bind or otherwise form complex with the target polynucleotide.

In one embodiment, the modifications of the polypeptide components may or may not cause an altered functionality. By means of example, modifications which do not result in an altered functionality include for instance codon optimization for expression into a particular host, or providing the polypeptide with a particular marker (e.g., for visualization). Modifications with may result in altered functionality may also include mutations, including point mutations, insertions, deletions, truncations (including split nucleases), etc., as well as chimeric nucleases (e.g., comprising domains from different orthologues or homologues) or fusion proteins. Fusion proteins may without limitation include, for instance, fusions with heterologous domains or functional domains (e.g., localization signals, catalytic domains, etc.). In one embodiment, various different modifications may be combined (e.g., a mutated nuclease which is catalytically inactive and which further is fused to a functional domain, such as for instance to induce DNA methylation or another nucleic acid modification, such as including without limitation, a break (e.g., by a different nuclease (domain)), a mutation, a deletion, an insertion, a replacement, a ligation, a digestion, a break or a recombination). As used herein, “altered functionality” includes without limitation an altered specificity (e.g., altered target recognition, increased or decreased specificity, or altered PAM recognition or insertion site recognition, altered activity (e.g., increased or decreased catalytic activity), and/or altered stability (e.g., fusions with destabilization domains). It will be understood that a “modified” polypeptide as referred to herein still has the capacity to interact with or bind to the polynucleic acid (e.g., in complex with the nucleic acid component molecule).

### Nuclear Localization Sequences

In one embodiment, one or more polypeptides in the CRISPR-Cas, Tn7-like transposase, or non-Tn7-like transposase may be fused with one or more nuclear localization sequences (NLSs), such as about or more than about 1, 2, 3, 4, 5, 6, 7, 8, 9, 10, or more NLSs. In one embodiment, the Fanzor polypeptide comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In a preferred embodiment of the invention, the Fanzor polypeptide comprises at most 6 NLSs. In one embodiment, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. Non-limiting examples of NLSs include an NLS sequence derived from: the NLS of the SV40 virus large T-antigen, having the amino acid sequence PKKKRKV (SEQ ID NO: 27); the NLS from nucleoplasmin (e.g., the nucleoplasmin bipartite NLS with the sequence KRPAATKKAGQAKKKK (SEQ ID NO: 28); the c-myc NLS having the amino acid sequence PAAKRVKLD (SEQ ID NO: 29) or RQRRNELKRSP (SEQ ID NO: 30); the hRNPA1 M9 NLS having the sequence NQSSNFGPMKGGNFGGRSSGPYGGGGQYFAKPRNQGGY (SEQ ID NO: 31); the sequence RMRIZFKNKGKDTAELRRRRVEVSVELRKAKKDEQILKRRN (SEQ ID NO: 32) of the IBB domain from importin-alpha; the sequences VSRKRPRP (SEQ ID NO: 33) and PPKKARED (SEQ ID NO: 34) of the myoma T protein; the sequence PQPKKKPL (SEQ ID NO: 35) of human p53; the sequence SALIKKKKKMAP (SEQ ID NO: 36) of mouse c-abl IV; the sequences DRLRR (SEQ ID NO: 37) and PKQKKRK (SEQ ID NO: 38) of the influenza virus NS1; the sequence RKLKKKIKKL (SEQ ID NO: 39) of the Hepatitis virus delta antigen; the sequence REKKKFLKRR (SEQ ID NO: 40) of the mouse Mx1 protein; the sequence KRKGDEVDGVDEVAKKKSKK (SEQ ID NO: 41) of the human poly(ADP-ribose) polymerase; and the sequence RKCLQAGMNLEARKTKK (SEQ ID NO: 42) of the steroid hormone receptors (human) glucocorticoid. In general, the one or more NLSs are of sufficient strength to drive accumulation of the polypeptide complexes in a detectable amount in the nucleus of a eukaryotic cell. In general, strength of nuclear localization activity may derive from the number of NLSs in the polypeptide, the particular NLS(s) used, or a combination of these factors. Detection of accumulation in the nucleus may be performed by any suitable technique. For example, a detectable marker may be fused to the polypeptide, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of complex formation (e.g., assay for DNA cleavage or mutation at the target sequence, or assay for altered gene expression activity affected by complex formation and/or polypeptide activity), as compared to a control no exposed to the Fanzor polypeptide or complex, or exposed to a polypeptide lacking the one or more NLSs. In one embodiment of the herein described polypeptide protein complexes and systems the codon optimized polypeptides comprise an NLS attached to the C-terminal of the protein. In one embodiment, other localization tags may be fused to the Fanzor polypeptide, such as without limitation for localizing the polypeptide to particular sites in a cell, such as organelles, such as mitochondria, plastids, chloroplast, vesicles, Golgi, (nuclear or cellular) membranes, ribosomes, nucleolus, ER, cytoskeleton, vacuoles, centrosome, nucleosome, granules, centrioles, etc.

In one embodiment of the invention, at least one nuclear localization signal (NLS) is attached to the nucleic acid sequences encoding the polypeptide. In preferred embodiments at least one or more C-terminal or N-terminal NLSs are attached (and hence nucleic acid molecule(s) coding for the polypeptide can include coding for NLS(s) so that the expressed product has the NLS(s) attached or connected). In a preferred embodiment a C-terminal NLS is attached for optimal expression and nuclear targeting in eukaryotic cells, preferably human cells. The invention also encompasses methods for delivering multiple nucleic acid components, wherein each nucleic acid component is specific for a different target locus of interest thereby modifying multiple target loci of interest. The nucleic acid component of the complex may comprise one or more protein-binding RNA aptamers. The one or more aptamers may be capable of binding a bacteriophage coat protein.

### Linkers

The term “associated with” is used here in relation to the association of the functional domain to the polypeptide protein or the adaptor protein. It is used in respect of how one molecule ‘associates’ with respect to another, for example between an adaptor protein and a functional domain, or between a Cas or transposase polypeptide protein and other components of the gene editing system. In the case of such protein-protein interactions, this association may be viewed in terms of recognition in the way an antibody recognizes an epitope. Alternatively, one protein may be associated with another protein via a fusion of the two, for instance one subunit being fused to another subunit. Fusion typically occurs by addition of the amino acid sequence of one to that of the other, for instance via splicing together of the nucleotide sequences that encode each protein or subunit. Alternatively, this may essentially be viewed as binding between two molecules or direct linkage, such as a fusion protein. In any event, the fusion protein may include a linker between the two subunits of interest (i.e., between the enzyme and the functional domain or between the adaptor protein and the functional domain). Thus, in one embodiment, the Fanzor polypeptide protein or adaptor protein is associated with a functional domain by binding thereto. In other embodiments, the polypeptide or adaptor protein is associated with a functional domain because the two are fused together, optionally via an intermediate linker.

The term “linker” as used in reference to a fusion protein refers to a molecule which joins the proteins to form a fusion protein. Generally, such molecules have no specific biological activity other than to join or to preserve some minimum distance or other spatial relationship between the proteins. However, in one embodiment, the linker may be selected to influence some property of the linker and/or the fusion protein such as the folding, net charge, or hydrophobicity of the linker.

Suitable linkers for use in the methods of the present invention are well known to those of skill in the art and include, but are not limited to, straight or branched-chain carbon linkers, heterocyclic carbon linkers, or peptide linkers. However, as used herein the linker may also be a covalent bond (carbon-carbon bond or carbon-heteroatom bond). In particular embodiments, the linker is used to separate the Fanzor polypeptide and the nucleotide deaminase by a distance sufficient to ensure that each protein retains its required functional property. Preferred peptide linker sequences adopt a flexible extended conformation and do not exhibit a propensity for developing an ordered secondary structure. In one embodiment, the linker can be a chemical moiety which can be monomeric, dimeric, multimeric or polymeric. Preferably, the linker comprises amino acids. Typical amino acids in flexible linkers include Gly, Asn and Ser. Accordingly, in particular embodiments, the linker comprises a combination of one or more of Gly, Asn and Ser amino acids. Other near neutral amino acids, such as Thr and Ala, also may be used in the linker sequence. Exemplary linkers are disclosed in Maratea et al. (1985), Gene 40:39-46; Murphy et al. (1986) Proc. Nat'l. Acad. Sci. USA 83:8258-62; U.S. Pat. Nos. 4,935,233; and 4,751,180. For example, GlySer linkers GGS, GGGS (SEQ ID NO: 43) or GSG can be used. GGS, GSG, GGGS (SEQ ID NO: 43) or GGGGS (SEQ ID NO: 44) linkers can be used in repeats of 3 (such as (GGS)₃ (SEQ ID NO: 45), (GGGGS)₃ (SEQ ID NO: 46) or 5, 6, 7, 9 or even 12 or more, to provide suitable lengths. In some cases, the linker may be (GGGGS)₃₋₁₅ (SEQ ID NO: 46-58), For example, in some cases, the linker may be (GGGGS)₃₋₁₁ (SEQ ID NO: 46-54), e.g., GGGGS (SEQ ID NO: 44), (GGGGS)₂ (SEQ ID NO: 59), (GGGGS)₃ (SEQ ID NO: 46), (GGGGS)₄(SEQ ID NO: 47), (GGGGS)₅ (SEQ ID NO: 48), (GGGGS)₆ (SEQ ID NO: 49), (GGGGS)₇ (SEQ ID NO: 50), (GGGGS)₈ (SEQ ID NO: 51), (GGGGS)₉ (SEQ ID NO: 52), (GGGGS)₁₀ (SEQ ID NO: 53), or (GGGGS)₁₁ (SEQ ID NO: 54).

In particular embodiments, linkers such as (GGGGS)₃ (SEQ ID NO: 46) are preferably used herein. (GGGGS)₆ (SEQ ID NO: 49), (GGGGS)₉ (SEQ ID NO: 52) or (GGGGS)₁₂ (SEQ ID NO: 55) may preferably be used as alternatives. Other preferred alternatives are (GGGGS)₁ (SEQ ID NO: 44), (GGGGS)₄(SEQ ID NO: 47), (GGGGS)₅ (SEQ ID NO: 48), (GGGGS)₇ (SEQ ID NO: 50), (GGGGS): (SEQ ID NO: 51), (GGGGS)₁₀ (SEQ ID NO: 53), or (GGGGS)₁₁ (SEQ ID NO: 54). In yet a further embodiment, LEPGEKPYKCPECGKSFSQSGALTRHQRTHTR (SEQ ID NO: 60) is used as a linker. In yet an additional embodiment, the linker is an XTEN linker. In particular embodiments, the Fanzor polypeptide is linked to the deaminase protein or its catalytic domain by means of an LEPGEKPYKCPECGKSFSQSGALTRHQRTHTR (SEQ ID NO: 60) (linker. In further particular embodiments, Fanzor polypeptide is linked C-terminally to the N-terminus of a deaminase protein or its catalytic domain by means of an LEPGEKPYKCPECGKSFSQSGALTRHQRTHTR ((SEQ ID NO: 60)) linker. In addition, N-and C-terminal NLSs can also function as linker (e.g., PKKKRKVEASSPKKRKVEAS (SEQ ID NO: 61)).

Examples of linkers are shown in Table 1 below.

TABLE 1

| | |
|---|---|
| GGS | GGTGGTAGT |
| GGS × 3 | GGTGGTAGTGGAGGGAGCGGCGGTTCA |
| (SEQ ID | (SEQ ID NO: 62) |
| NO: 45) | |
| GGS × 7 | ggtggaggaggctctggtggaggcggt |
| (SEQ ID | agcggaggcggagggtcgGGTGGTAGT |
| NO: 63) | GGAGGGAGCGGCGGTTCA |
| | (SEQ ID NO: 64) |
| XTEN | TCGGGATCTGAGACGCCTGGGACCTC |
| | GGAATCGGCTACGCCCGAAAGT |
| | (SEQ ID NO: 65) |
| Z-EFGR_ | Gtggataacaaatttaacaaagaaat |
| Short | gtgggcggcgtgggaagaaattcgta |
| | acctgccgaacctgaacggctggcag |
| | atgaccgcgtttattgcgagcctggt |
| | ggatgatccgagccagagcgcgaacc |
| | tgctggcggaagcgaaaaaactgaac |
| | gatgcgcaggcgccgaaaaccggcgg |
| | tggttctggt (SEQ ID NO: 66) |
| GSAT | Ggtggttctgccggtggctccggttc |
| | tggctccagcggtggcagctctggtg |
| | cgtccggcacgggtactgcgggtggc |
| | actggcagcggttccggtactggctc |
| | tggc (SEQ ID NO: 67) |

Linkers may be used between the Nucleic acid component molecules and the functional domain (activator or repressor), or between the Fanzor polypeptide and the functional domain. The linkers may be used to engineer appropriate amounts of “mechanical flexibility”.

In one embodiment, the one or more functional domains are controllable, e.g., inducible.

Other suitable functional domains can be found, for example, in International Application Publication No. WO 2019/018423, for example, at [0678]-[0692], incorporated herein by reference. Exemplary functional domains are further detailed elsewhere herein.

### Polynucleotides and Vectors

The systems herein may comprise one or more polynucleotides. The polynucleotide(s) may comprise coding sequences of Cas protein(s), transposase(s), guide molecule(s), donor polynucleotide(s), or any combination thereof. The polynucleotide(s) may also comprise coding sequences of tyrosine recombinases, HTH domain proteins, GIY-YIG nucleases, transposase(s), Cas protein(s), guide molecule(s), donor polynucleotide(s), or any combination thereof. The present disclosure further provides vectors or vector systems comprising one or more polynucleotides herein. The vectors or vector systems include those described in the delivery sections herein.

The terms “polynucleotide”, “nucleotide”, “nucleotide sequence”, “nucleic acid”, and “oligonucleotide” are used interchangeably herein. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. The term also encompasses nucleic acid-like structures with synthetic backbones, see, e.g., Eckstein, 1991; Baserga et al., 1992; Milligan, 1993; WO 97/03211; WO 96/39154; Mata, 1997; Strauss-Soukup, 1997; and Samstag, 1996. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. As used herein the term “wild type” is a term of the art understood by skilled persons and means the typical form of an organism, strain, gene or characteristic as it occurs in nature as distinguished from mutant or variant forms. A “wild type” can be a base line. As used herein the term “variant” should be taken to mean the exhibition of qualities that have a pattern that deviates from what occurs in nature. The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature. “Complementarity” refers to the ability of a nucleic acid to form hydrogen bond(s) with another nucleic acid sequence by either traditional Watson-Crick base pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions. As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part I, Second Chapter “Overview of principles of hybridization and the strategy of nucleic acid probe assay”, Elsevier, N.Y. Where reference is made to a polynucleotide sequence, then complementary or partially complementary sequences are also envisaged. These are preferably capable of hybridizing to the reference sequence under highly stringent conditions. Generally, in order to maximize the hybridization rate, relatively low-stringency hybridization conditions are selected: about 20 to 25° C. lower than the thermal melting point (Tm). The Tm is the temperature at which 50% of specific target sequence hybridizes to a perfectly complementary probe in solution at a defined ionic strength and pH. Generally, in order to require at least about 85% nucleotide complementarity of hybridized sequences, highly stringent washing conditions are selected to be about 5 to 15° C. lower than the Tm. A sequence capable of hybridizing with a given sequence is referred to as the “complement” of the given sequence.

As used herein, the term “genomic locus” or “locus” (plural loci) is the specific location of a gene or DNA sequence on a chromosome. A “gene” refers to stretches of DNA or RNA that encode a polypeptide or an RNA chain that has functional role to play in an organism and hence is the molecular unit of heredity in living organisms. It may be considered that genes include regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions. As used herein, “expression of a genomic locus” or “gene expression” is the process by which information from a gene is used in the synthesis of a functional gene product. The products of gene expression are often proteins, but in non-protein coding genes such as rRNA genes or tRNA genes, the product is functional RNA. The process of gene expression is used by all known life-eukaryotes (including multicellular organisms), prokaryotes (bacteria and archaea) and viruses to generate functional products to survive. As used herein “expression” of a gene or nucleic acid encompasses not only cellular gene expression, but also the transcription and translation of nucleic acid(s) in cloning systems and in any other context. As used herein, “expression” also refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell. The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics. As used herein, the term “domain” or “protein domain” refers to a part of a protein sequence that may exist and function independently of the rest of the protein chain. As described in aspects, sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences.

In certain embodiments, the polynucleotide sequence is recombinant DNA. In further embodiments, the polynucleotide sequence further comprises additional sequences as described elsewhere herein. In certain embodiments, the nucleic acid sequence is synthesized in vitro.

Aspects of the disclosure relate to polynucleotide molecules that encode one or more components of the systems as referred to in any embodiment herein. In certain embodiments, the polynucleotide molecules may comprise further regulatory sequences. By means of guidance and not limitation, the polynucleotide sequence can be part of an expression plasmid, a minicircle, a lentiviral vector, a retroviral vector, an adenoviral or adeno-associated viral vector, a piggyback vector, or a tol2 vector. In certain embodiments, the polynucleotide sequence may be a bicistronic expression construct. In further embodiments, the isolated polynucleotide sequence may be incorporated in a cellular genome. In yet further embodiments, the isolated polynucleotide sequence may be part of a cellular genome. In further embodiments, the isolated polynucleotide sequence may be comprised in an artificial chromosome. In certain embodiments, the 5′ and/or 3′ end of the isolated polynucleotide sequence may be modified to improve the stability of the sequence of actively avoid degradation. In certain embodiments, the isolated polynucleotide sequence may be comprised in a bacteriophage. In other embodiments, the isolated polynucleotide sequence may be contained in agrobacterium species. In certain embodiments, the isolated polynucleotide sequence is lyophilized.

### Codon Optimization

Aspects of the disclosure relate to polynucleotide molecules that encode one or more components of the systems as described in any of the embodiments herein, wherein at least one or more regions of the polynucleotide molecule may be codon optimized for expression in a eukaryotic cell. In certain embodiments, the polynucleotide molecules that encode one or more components of the systems as described in any of the embodiments herein are optimized for expression in a mammalian cell or a plant cell.

An example of a codon optimized sequence, is in this instance a sequence optimized for expression in a eukaryote, e.g., humans (i.e., being optimized for expression in humans), or for another eukaryote, animal or mammal as herein discussed; see, e.g., SaCas9 human codon optimized sequence in International Patent Publication No. WO 2014/093622 (PCT/US2013/074667) as an example of a codon optimized sequence (from knowledge in the art and this disclosure, codon optimizing coding nucleic acid molecule(s), especially as to effector protein is within the ambit of the skilled artisan). Whilst this is preferred, it will be appreciated that other examples are possible and codon optimization for a host species other than human, or for codon optimization for specific organs is known. In some embodiments, an enzyme coding sequence encoding a Cas protein and/or transposase is codon optimized for expression in particular cells, such as eukaryotic cells. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate. In some embodiments, processes for modifying the germ line genetic identity of human beings and/or processes for modifying the genetic identity of animals which are likely to cause them suffering without any substantial medical benefit to man or animal, and also animals resulting from such processes, may be excluded. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In some embodiments, one or more codons (e.g., 1, 2, 3, 4, 5, 10, 15, 20, 25, 50, or more, or all codons) in a sequence encoding a DNA/RNA-targeting Cas protein corresponds to the most frequently used codon for a particular amino acid.

### Method of Inserting Polynucleotides

The present disclosure further provides methods of inserting a polynucleotide into a target nucleic acid in a cell, which comprises introducing into a cell one or more of the engineered systems described herein. In some examples, the present disclosure provides methods of inserting a donor polynucleotide into a target polynucleotide in a cell, said method comprising introducing into the cell an engineered system comprising one or more Type I-D Cas proteins, one or more CRISPR-associated Tn7 transposases or functional fragments thereof linked to or otherwise capable of associated with the one or more Type I-D Cas proteins, and a guide molecule capable of forming a complex with the one or more Type I-D Cas proteins and directing sequence-specific binding of the complex to a target polynucleotide. In some examples, the method of inserting a donor polynucleotide into a target polynucleotide in a cell comprises introducing into the cell an engineered system comprising one or more Tn7 transposases or functional fragments thereof (e.g., TnsA, TnsB, TnsC, and TnsF; TnsAB, TnsC, and TnsF; etc.). In some examples, the method of inserting a donor polynucleotide into a target polynucleotide in a cell comprises introducing into the cell an engineered system comprising one or more tyrosine recombinases, one or more HTH domain proteins, and one or more TnsF homologs.

The one or more of components of the engineered systems described herein may be expressed from a nucleic acid operably linked to a regulatory sequence that is expressed in the cell. The one or more of components of the engineered systems described herein may be introduced into a particle for delivery into the cell. The particle may comprise a ribonucleoprotein (RNP). The cell may be a prokaryotic cell. The cell may be a eukaryotic cell. The cell may be a mammalian cell, a cell of a non-human primate, or a human cell. The cell may be a plant cell.

In some cases, the method of inserting a donor polynucleotide into a target polynucleotide in a cell, which comprises introducing into the cell: one or more transposases (e.g., CRISPR-associated transposases), a Cas protein; and a guide molecule capable of complexing with the Cas protein and directing sequence specific binding of the guide-Cas protein complex to a target sequence of the target nucleic acid. The one or more CRISPR-associated transposons may comprise one or more transposases and a donor polynucleotide to be inserted.

### Immune Orthogonal Orthologs

In some embodiments, when one or more components of the systems (e.g., transposases, nucleotide-binding molecules) herein need to be expressed or administered in a subject, immunogenicity of the components may be reduced by sequentially expressing or administering immune orthogonal orthologs of the components of the transposon complexes to the subject. As used herein, the term “immune orthogonal orthologs” refer to orthologous proteins that have similar or substantially the same function or activity, but have no or low cross-reactivity with the immune response generated by one another. In some embodiments, sequential expression or administration of such orthologs elicits low or no secondary immune response. The immune orthogonal orthologs can avoid being neutralized by antibodies (e.g., existing antibodies in the host before the orthologs are expressed or administered). Cells expressing the orthologs can avoid being cleared by the host's immune system (e.g., by activated CTLs). In some examples, CRISPR enzyme orthologs from different species may be immune orthogonal orthologs.

Immune orthogonal orthologs may be identified by analyzing the sequences, structures, and/or immunogenicity of a set of candidates orthologs. In an example method, a set of immune orthogonal orthologs may be identified by a) comparing the sequences of a set of candidate orthologs (e.g., orthologs from different species) to identify a subset of candidates that have low or no sequence similarity; b) assessing immune overlap among the members of the subset of candidates to identify candidates that have no or low immune overlap. In some cases, immune overlap among candidates may be assessed by determining the binding (e.g., affinity) between a candidate ortholog and MHC (e.g., MHC type I and/or MHC II) of the host. Alternatively or additionally, immune overlap among candidates may be assessed by determining B-cell epitopes for the candidate orthologs. In one example, immune orthogonal orthologs may be identified using the method described in Moreno A M et al., BioRxiv, published online Jan. 10, 2018, doi: doi.org/10.1101/245985.

### Delivery

The present disclosure also provides delivery systems for introducing components of the systems and compositions herein to cells, tissues, organs, or organisms. A delivery system may comprise one or more delivery vehicles and/or cargos. Exemplary delivery systems and methods include those described in paragraphs to of Feng Zhang et al., (WO2016106236A1), and pages 1241-1251 and Table 1 of Lino C A et al., Delivering CRISPR: a review of the challenges and approaches, DRUG DELIVERY, 2018, VOL. 25, NO. 1, 1234-1257, which are incorporated by reference herein in their entireties.

In some embodiments, the delivery systems may be used to introduce the components of the systems and compositions to plant cells. For example, the components may be delivered to plant using electroporation, microinjection, aerosol beam injection of plant cell protoplasts, biolistic methods, DNA particle bombardment, and/or Agrobacterium-mediated transformation. Examples of methods and delivery systems for plants include those described in Fu et al., Transgenic Res. 2000 February; 9 (1): 11-9; Klein R M, et al., Biotechnology. 1992; 24:384-6; Casas A M et al., Proc Natl Acad Sci USA. 1993 Dec. 1; 90 (23): 11212-11216; and U.S. Pat. No. 5,563,055, Davey M R et al., Plant Mol Biol. 1989 September; 13 (3): 273-85, which are incorporated by reference herein in their entireties.

### Cargos

The delivery systems may comprise one or more cargos. The cargos may comprise one or more components of the engineered systems described herein. In some embodiments, a cargo may comprise one or more plasmids encoding one or more Cas proteins; one or more plasmids encoding one or more transposases; one or more plasmids encoding one or more guide molecules; or any combination thereof. In some embodiments, a cargo may comprise one or more plasmids encoding one or more tyrosine recombinases; one or more plasmids encoding one or more HTH domain proteins; one or more plasmids encoding one or more TnsF homologs; or any combination thereof. In some examples, a cargo may comprise a plasmid encoding one or more Cas protein and one or more (e.g., a plurality of) guide RNAs. In some embodiments, a cargo may comprise mRNA encoding one or more Cas proteins and one or more guide RNAs.

In some examples, a cargo may comprise one or more Cas proteins and one or more guide RNAs, e.g., in the form of ribonucleoprotein complexes (RNPs). The ribonucleoprotein complexes may be delivered by methods and systems herein. In some cases, the ribonucleoprotein may be delivered by way of a polypeptide-based shuttle agent. In one example, the ribonucleoprotein may be delivered using synthetic peptides comprising an endosome leakage domain (ELD) operably linked to a cell penetrating domain (CPD), to a histidine-rich domain and a CPD, e.g., as describe in WO2016161516. RNP may also be used for delivering the compositions and systems to plant cells, e.g., as described in Wu J W, et al., Nat Biotechnol. 2015 November; 33 (11): 1162-4.

### Physical Delivery

In some embodiments, the cargos may be introduced to cells by physical delivery methods. Examples of physical methods include microinjection, electroporation, and hydrodynamic delivery. Both nucleic acid and proteins may be delivered using such methods. For example, Cas protein may be prepared in vitro, isolated, (refolded, purified if needed), and introduced to cells.

### Microinjection

Microinjection of the cargo directly to cells can achieve high efficiency, e.g., above 90% or about 100%. In some embodiments, microinjection may be performed using a microscope and a needle (e.g., with 0.5-5.0 μm in diameter) to pierce a cell membrane and deliver the cargo directly to a target site within the cell. Microinjection may be used for in vitro and ex vivo delivery.

Plasmids comprising coding sequences for Cas proteins and/or guide RNAs, mRNAs, and/or guide RNAs, may be microinjected. In some cases, microinjection may be used i) to deliver DNA directly to a cell nucleus, and/or ii) to deliver mRNA (e.g., in vitro transcribed) to a cell nucleus or cytoplasm. In certain examples, microinjection may be used to delivery sgRNA directly to the nucleus and Cas-encoding mRNA to the cytoplasm, e.g., facilitating translation and shuttling of Cas to the nucleus.

Microinjection may be used to generate genetically modified animals. For example, gene editing cargos may be injected into zygotes to allow for efficient germline modification. Such approach can yield normal embryos and full-term mouse pups harboring the desired modification(s). Microinjection can also be used to provide transiently up- or down-regulate a specific gene within the genome of a cell, e.g., using CRISPRa and CRISPRi.

### Electroporation

In some embodiments, the cargos and/or delivery vehicles may be delivered by electroporation. Electroporation may use pulsed high-voltage electrical currents to transiently open nanometer-sized pores within the cellular membrane of cells suspended in buffer, allowing for components with hydrodynamic diameters of tens of nanometers to flow into the cell. In some cases, electroporation may be used on various cell types and efficiently transfer cargo into cells. Electroporation may be used for in vitro and ex vivo delivery.

Electroporation may also be used to deliver the cargo to into the nuclei of mammalian cells by applying specific voltage and reagents, e.g., by nucleofection. Such approaches include those described in Wu Y, et al. (2015). Cell Res 25:67-79; Ye L, et al. (2014). Proc Natl Acad Sci USA 111:9591-6; Choi P S, Meyerson M. (2014). Nat Commun 5:3728; Wang J, Quake S R. (2014). Proc Natl Acad Sci 111:13157-62. Electroporation may also be used to deliver the cargo in vivo, e.g., with methods described in Zuckermann M, et al. (2015). Nat Commun 6:7391.

### HYDRODYNAMIC Delivery

Hydrodynamic delivery may also be used for delivering the cargos, e.g., for in vivo delivery. In some examples, hydrodynamic delivery may be performed by rapidly pushing a large volume (8-10% body weight) solution containing the gene editing cargo into the bloodstream of a subject (e.g., an animal or human), e.g., for mice, via the tail vein. As blood is incompressible, the large bolus of liquid may result in an increase in hydrodynamic pressure that temporarily enhances permeability into endothelial and parenchymal cells, allowing for cargo not normally capable of crossing a cellular membrane to pass into cells. This approach may be used for delivering naked DNA plasmids and proteins. The delivered cargos may be enriched in liver, kidney, lung, muscle, and/or heart.

### Transfection

The cargos, e.g., nucleic acids, may be introduced to cells by transfection methods for introducing nucleic acids into cells. Examples of transfection methods include calcium phosphate-mediated transfection, cationic transfection, liposome transfection, dendrimer transfection, heat shock transfection, magnetofection, lipofection, impalefection, optical transfection, proprietary agent-enhanced uptake of nucleic acid.

### Delivery Vehicles

The delivery systems may comprise one or more delivery vehicles. The delivery vehicles may deliver the cargo into cells, tissues, organs, or organisms (e.g., animals or plants). The cargos may be packaged, carried, or otherwise associated with the delivery vehicles. The delivery vehicles may be selected based on the types of cargo to be delivered, and/or the delivery is in vitro and/or in vivo. Examples of delivery vehicles include vectors, viruses, non-viral vehicles, and other delivery reagents described herein.

The delivery vehicles in accordance with the present invention may a greatest dimension (e.g., diameter) of less than 100 microns (μm). In some embodiments, the delivery vehicles have a greatest dimension of less than 10 μm. In some embodiments, the delivery vehicles may have a greatest dimension of less than 2000 nanometers (nm). In some embodiments, the delivery vehicles may have a greatest dimension of less than 1000 nanometers (nm). In some embodiments, the delivery vehicles may have a greatest dimension (e.g., diameter) of less than 900 nm, less than 800 nm, less than 700 nm, less than 600 nm, less than 500 nm, less than 400 nm, less than 300 nm, less than 200 nm, less than 150 nm, or less than 100 nm, less than 50 nm. In some embodiments, the delivery vehicles may have a greatest dimension ranging between 25 nm and 200 nm.

In some embodiments, the delivery vehicles may be or comprise particles. For example, the delivery vehicle may be or comprise nanoparticles (e.g., particles with a greatest dimension (e.g., diameter) no greater than 1000 nm. The particles may be provided in different forms, e.g., as solid particles (e.g., metal such as silver, gold, iron, titanium), non-metal, lipid-based solids, polymers), suspensions of particles, or combinations thereof. Metal, dielectric, and semiconductor particles may be prepared, as well as hybrid structures (e.g., core-shell particles). Nanoparticles may also be used to deliver the compositions and systems to plant cells, e.g., as described in WO 2008042156, US20130185823, and WO2015089419.

### Vectors

The systems, compositions, and/or delivery systems may comprise one or more vectors. The present disclosure also includes vector systems. A vector system may comprise one or more vectors. In some embodiments, a vector refers to a nucleic acid molecule capable of transporting another nucleic acid to which it has been linked. Vectors include nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. A vector may be a plasmid, e.g., a circular double stranded DNA loop into which additional DNA segments can be inserted, such as by standard molecular cloning techniques. Certain vectors may be capable of autonomous replication in a host cell into which they are introduced (e.g., bacterial vectors having a bacterial origin of replication and episomal mammalian vectors). Some vectors (e.g., non-episomal mammalian vectors) are integrated into the genome of a host cell upon introduction into the host cell, and thereby are replicated along with the host genome. In certain examples, vectors may be expression vectors, e.g., capable of directing the expression of genes to which they are operatively-linked. In some cases, the expression vectors may be for expression in eukaryotic cells. Common expression vectors of utility in recombinant DNA techniques are often in the form of plasmids.

Examples of vectors include pGEX, pMAL, pRIT5, E. coli expression vectors (e.g., pTrc, pET 11d, yeast expression vectors (e.g., pYepSec1, pMFa, pJRY88, pYES2, and picZ, Baculovirus vectors (e.g., for expression in insect cells such as SF9 cells) (e.g., pAc series and the pVL series), mammalian expression vectors (e.g., pCDM8 and pMT2PC).

A vector may comprise i) Cas encoding sequence(s), and/or ii) a single, or at least 2, at least 3, at least 4, at least 5, at least 6, at least 7, at least 8, at least 9, at least 10, at least 12, at least 14, at least 16, at least 32, at least 48, at least 50 guide RNA(s) encoding sequences. In a single vector there can be a promoter for each RNA coding sequence. Alternatively or additionally, in a single vector, there may be a promoter controlling (e.g., driving transcription and/or expression) multiple RNA encoding sequences.

In some embodiments, the components (or coding sequences thereof) of any of the engineered systems described herein may be comprised in a single vector. In some examples, a single vector may comprise coding sequences for one or more CRISPR-associated Tn7 transposases, one or more Cas proteins, and one or more guide molecules. In some examples, a single vector may comprise coding sequences for one or more CRISPR-associated Tn7 transposases. In some examples, a single vector may comprise coding sequences for one or more tyrosine recombinases, one or more HTH domain proteins, and one or more TnsF homologs. In certain embodiments, the components (or coding sequences thereof) of any of the engineered systems described herein may be comprised in separate vectors. In some examples, a first vector may comprise coding sequences for one or more CRISPR-associated Tn7 transposases; a second vector may comprise coding sequences for one or more Cas proteins; a third vector may comprise coding sequences for one or more guide molecules. In some examples, a first vector may comprise coding sequences for one or more CRISPR-associated Tn7 transposases and one or more Cas proteins; a second vector may comprise coding sequences for one or more guide molecules. In some examples, a first vector may comprise coding sequences for one or more CRISPR-associated Tn7 transposases; a second vector may comprise coding sequences for one or more Cas proteins and one or more guide molecules. In some examples, a first vector may comprise coding sequences for one or more CRISPR-associated Tn7 transposases and one or more guide molecules; a second vector may comprise coding sequences for one or more Cas proteins. In some examples, a first vector may comprise coding sequences for one or more tyrosine recombinases; a second vector may comprise coding sequences for one or more HTH domain proteins; and a third vector may comprise coding sequences for one or more TnsF homologs. In some examples, a first vector may comprise coding sequences for one or more tyrosine recombinases and one or more HTH domain proteins; and a second vector may comprise coding sequences for one or more TnsF homologs. In some examples, a first vector may comprise coding sequences for one or more tyrosine recombinases; and a second vector may comprise coding sequences for one or more HTH domain proteins and one or more TnsF homologs. In some examples, a first vector may comprise coding sequences for one or more tyrosine recombinases and one or more TnsF homologs; and a second vector may comprise coding sequences for one or more HTH domain proteins.

### Regulatory Elements

A vector may comprise one or more regulatory elements. The regulatory element(s) may be operably linked to coding sequences of Cas proteins, transposases, tyrosine recombinases, HTH domain proteins, GIY-YIG nucleases, accessary proteins, guide RNAs (e.g., a single guide RNA, crRNA, and/or tracrRNA), or combination thereof. The term “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host celD. In certain examples, a vector may comprise a first regulatory element operably linked to a nucleotide sequence encoding a Cas protein, and a second regulatory element operably linked to a nucleotide sequence encoding a guide RNA.

Examples of regulatory elements include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific.

Examples of promoters include one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the β-actin promoter, the phosphoglycerol kinase (PGK) promoter, and the EF1α promoter.

### Viral Vectors

The cargos may be delivered by viruses. In some embodiments, viral vectors are used. A viral vector may comprise virally derived DNA or RNA sequences for packaging into a virus (e.g., retroviruses, replication defective retroviruses, adenoviruses, replication defective adenoviruses, and adeno-associated viruses). Viral vectors also include polynucleotides carried by a virus for transfection into a host cell. Viruses and viral vectors may be used for in vitro, ex vivo, and/or in vivo deliveries.

### Adeno Associated Virus (AAV)

The systems and compositions herein may be delivered by adeno associated virus (AAV). AAV vectors may be used for such delivery. AAV, of the Dependovirus genus and Parvoviridae family, is a single stranded DNA virus. In some embodiments, AAV may provide a persistent source of the provided DNA, as AAV delivered genomic material can exist indefinitely in cells, e.g., either as exogenous DNA or, with some modification, be directly integrated into the host DNA. In some embodiments, AAV do not cause or relate with any diseases in humans. The virus itself is able to efficiently infect cells while provoking little to no innate or adaptive immune response or associated toxicity.

Examples of AAV that can be used herein include AAV-1, AAV-2, AAV-3, AAV-4, AAV-5, AAV-6, AAV-8, and AAV-9. The type of AAV may be selected with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. AAV-2-based vectors were originally proposed for CFTR delivery to CF airways, other serotypes such as AAV-1, AAV-5, AAV-6, and AAV-9 exhibit improved gene transfer efficiency in a variety of models of the lung epithelium. Examples of cell types targeted by AAV are described in Grimm, D. et al, J. Virol. 82:5887-5911 (2008), and shown as follows:

TABLE 2

| Cell Line | AAV-1 | AAV-2 | AAV-3 | AAV-4 | AAV-5 | AAV-6 | AAV-8 | AAV-9 |
|---|---|---|---|---|---|---|---|---|
| Huh-7 | 13 | 100 | 2.5 | 0.0 | 0.1 | 10 | 0.7 | 0.0 |
| HEK293 | 25 | 100 | 2.5 | 0.1 | 0.1 | 5 | 0.7 | 0.1 |
| HeLa | 3 | 100 | 2.0 | 0.1 | 6.7 | 1 | 0.2 | 0.1 |
| HepG2 | 3 | 100 | 16.7 | 0.3 | 1.7 | 5 | 0.3 | ND |
| Hep1A | 20 | 100 | 0.2 | 1.0 | 0.1 | 1 | 0.2 | 0.0 |
| 911 | 17 | 100 | 11 | 0.2 | 0.1 | 17 | 0.1 | ND |
| CHO | 100 | 100 | 14 | 1.4 | 333 | 50 | 10 | 1.0 |
| COS | 33 | 100 | 33 | 3.3 | 5.0 | 14 | 2.0 | 0.5 |
| MeWo | 10 | 100 | 20 | 0.3 | 6.7 | 10 | 1.0 | 0.2 |
| NIH3T3 | 10 | 100 | 2.9 | 2.9 | 0.3 | 10 | 0.3 | ND |
| A549 | 14 | 100 | 20 | ND | 0.5 | 10 | 0.5 | 0.1 |
| HT1180 | 20 | 100 | 10 | 0.1 | 0.3 | 33 | 0.5 | 0.1 |
| Monocytes | 1111 | 100 | ND | ND | 125 | 1429 | ND | ND |
| Immature DC | 2500 | 100 | ND | ND | 222 | 2857 | ND | ND |
| Mature DC | 2222 | 100 | ND | ND | 333 | 3333 | ND | ND |

AAV particles may be created in HEK 293 T cells. Once particles with specific tropism have been created, they are used to infect the target cell line much in the same way that native viral particles do. This may allow for persistent presence of CRISPR-Cas components in the infected cell type, and what makes this version of delivery particularly suited to cases where long-term expression is desirable. Examples of doses and formulations for AAV that can be used include those describe in U.S. Pat. Nos. 8,454,972 and 8,404,658.

Various strategies may be used for delivery the systems and compositions herein with AAVs. In some examples, coding sequences of Cas and gRNA may be packaged directly onto one DNA plasmid vector and delivered via one AAV particle. In some examples, AAVs may be used to deliver gRNAs into cells that have been previously engineered to express Cas. In some examples, coding sequences of Cas and gRNA may be made into two separate AAV particles, which are used for co-transfection of target cells. In some examples, markers, tags, and other sequences may be packaged in the same AAV particles as coding sequences of Cas and/or gRNAs.

### Lentiviruses

The systems and compositions herein may be delivered by lentiviruses. Lentiviral vectors may be used for such delivery. Lentiviruses are complex retroviruses that have the ability to infect and express their genes in both mitotic and post-mitotic cells.

Examples of lentiviruses include human immunodeficiency virus (HIV), which may use its envelope glycoproteins of other viruses to target a broad range of cell types; minimal non-primate lentiviral vectors based on the equine infectious anemia virus (EIAV), which may be used for ocular therapies. In certain embodiments, self-inactivating lentiviral vectors with an siRNA targeting a common exon shared by HIV tat/rev, a nucleolar-localizing TAR decoy, and an anti-CCR5-specific hammerhead ribozyme (see, e.g., DiGiusto et al. (2010) Sci Transl Med 2: 36ra43) may be used/and or adapted to the nucleic acid-targeting system herein.

Lentiviruses may be pseudo-typed with other viral proteins, such as the G protein of vesicular stomatitis virus. In doing so, the cellular tropism of the lentiviruses can be altered to be as broad or narrow as desired. In some cases, to improve safety, second-and third-generation lentiviral systems may split essential genes across three plasmids, which may reduce the likelihood of accidental reconstitution of viable viral particles within cells.

In some examples, leveraging the integration ability, lentiviruses may be used to create libraries of cells comprising various genetic modifications, e.g., for screening and/or studying genes and signaling pathways.

### Adenoviruses

The systems and compositions herein may be delivered by adenoviruses. Adenoviral vectors may be used for such delivery. Adenoviruses include nonenveloped viruses with an icosahedral nucleocapsid containing a double stranded DNA genome. Adenoviruses may infect dividing and non-dividing cells. In some embodiments, adenoviruses do not integrate into the genome of host cells, which may be used for limiting off-target effects of CRISPR-Cas systems in gene editing applications.

### Viral Vehicles for Delivery to Plants

The systems and compositions may be delivered to plant cells using viral vehicles. In particular embodiments, the compositions and systems may be introduced in the plant cells using a plant viral vector (e.g., as described in Scholthof et al. 1996, Annu Rev Phytopathol. 1996; 34:299-323). Such viral vector may be a vector from a DNA virus, e.g., geminivirus (e.g., cabbage leaf curl virus, bean yellow dwarf virus, wheat dwarf virus, tomato leaf curl virus, maize streak virus, tobacco leaf curl virus, or tomato golden mosaic virus) or nanovirus (e.g., Faba bean necrotic yellow virus). The viral vector may be a vector from an RNA virus, e.g., tobravirus (e.g., tobacco rattle virus, tobacco mosaic virus), potexvirus (e.g., potato virus X), or hordeivirus (e.g., barley stripe mosaic virus). The replicating genomes of plant viruses may be non-integrative vectors.

### Non-Viral Vehicles

The delivery vehicles may comprise non-viral vehicles. In general, methods and vehicles capable of delivering nucleic acids and/or proteins may be used for delivering the systems compositions herein. Examples of non-viral vehicles include lipid nanoparticles, cell-penetrating peptides (CPPs), DNA nanoclews, gold nanoparticles, streptolysin O, multifunctional envelope-type nanodevices (MENDs), lipid-coated mesoporous silica particles, and other inorganic nanoparticles.

### Lipid Particles

The delivery vehicles may comprise lipid particles, e.g., lipid nanoparticles (LNPs) and liposomes.

### Lipid Nanoparticles (LNPs)

LNPs may encapsulate nucleic acids within cationic lipid particles (e.g., liposomes), and may be delivered to cells with relative ease. In some examples, lipid nanoparticles do not contain any viral components, which helps minimize safety and immunogenicity concerns. Lipid particles may be used for in vitro, ex vivo, and in vivo deliveries. Lipid particles may be used for various scales of cell populations.

In some examples. LNPs may be used for delivering DNA molecules (e.g., those comprising coding sequences of Cas and/or gRNA) and/or RNA molecules (e.g., mRNA of Cas, gRNAs). In certain cases, LNPs may be use for delivering RNP complexes of Cas/gRNA.

Components in LNPs may comprise cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N,N-dimethyl-3-aminopropane (DLinK-DMA), 1,2-dilinoleyl-4-(2-dimethylaminoethyD-[1,3]-dioxolane (DLinKC2-DMA), (3-o-[2″-(methoxypolyethyleneglycol 2000) succinoyl]-1,2-dimyristoyl-sn-glycol (PEG-S-DMG), R-3-[(ro-methoxy-poly(ethylene glycoD 2000) carbamoyl]-1,2-dimyristyloxlpropyl-3-amine (PEG-C-DOMG, and any combination thereof. Preparation of LNPs and encapsulation may be adapted from Rosin et al, Molecular Therapy, vol. 19, no. 12, pages 1286-220 Dec. 2011).

### Liposomes

In some embodiments, a lipid particle may be liposome. Liposomes are spherical vesicle structures composed of a uni- or multilamellar lipid bilayer surrounding internal aqueous compartments and a relatively impermeable outer lipophilic phospholipid bilayer. In some embodiments, liposomes are biocompatible, nontoxic, can deliver both hydrophilic and lipophilic drug molecules, protect their cargo from degradation by plasma enzymes, and transport their load across biological membranes and the blood brain barrier (BBB).

Liposomes can be made from several different types of lipids, e.g., phospholipids. A liposome may comprise natural phospholipids and lipids such as 1,2-distearoryl-sn-glycero-3-phosphatidyl choline (DSPC), sphingomyelin, egg phosphatidylcholines, monosialoganglioside, or any combination thereof.

Several other additives may be added to liposomes in order to modify their structure and properties. For instance, liposomes may further comprise cholesterol, sphingomyelin, and/or 1,2-dioleoyl-sn-glycero-3-phosphoethanolamine (DOPE), e.g., to increase stability and/or to prevent the leakage of the liposomal inner cargo.

### Stable Nucleic-Acid-Lipid Particles (SNALPs)

In some embodiments, the lipid particles may be stable nucleic acid lipid particles (SNALPs). SNALPs may comprise an ionizable lipid (DLinDMA) (e.g., cationic at low pH), a neutral helper lipid, cholesterol, a diffusible polyethylene glycol (PEG)-lipid, or any combination thereof. In some examples, SNALPs may comprise synthetic cholesterol, dipalmitoylphosphatidylcholine, 3-N-[(w-methoxy polyethylene glycoD 2000) carbamoyl]-1,2-dimyrestyloxypropylamine, and cationic 1,2-dilinoleyloxy-3-N,Ndimethylaminopropane. In some examples, SNALPs may comprise synthetic cholesterol, 1,2-distearoyl-sn-glycero-3-phosphocholine, PEG-CDMA, and 1,2-dilinoleyloxy-3-(N;N-dimethyD aminopropane (DLinDMA).

### Other Lipids

The lipid particles may also comprise one or more other types of lipids, e.g., cationic lipids, such as amino lipid 2,2-dilinoleyl-4-dimethylaminoethyl-[1,3]-dioxolane (DLin-KC2-DMA), DLin-KC2-DMA4, C12-200 and colipids disteroylphosphatidyl choline, cholesterol, and PEG-DMG.

### Lipoplexes/Polyplexes

In some embodiments, the delivery vehicles comprise lipoplexes and/or polyplexes. Lipoplexes may bind to negatively charged cell membrane and induce endocytosis into the cells. Examples of lipoplexes may be complexes comprising lipid(s) and non-lipid components. Examples of lipoplexes and polyplexes include FuGENE-6 reagent, a non-liposomal solution containing lipids and other components, zwitterionic amino lipids (ZALs), Ca2p (e.g., forming DNA/Ca2· microcomplexes), polyethenimine (PEI) (e.g., branched PEI), and poly(L-lysine) (PLL).

### Cell Penetrating Peptides

In some embodiments, the delivery vehicles comprise cell penetrating peptides (CPPs). CPPs are short peptides that facilitate cellular uptake of various molecular cargo (e.g., from nanosized particles to small chemical molecules and large fragments of DNA).

CPPs may be of different sizes, amino acid sequences, and charges. In some examples, CPPs can translocate the plasma membrane and facilitate the delivery of various molecular cargoes to the cytoplasm or an organelle. CPPs may be introduced into cells via different mechanisms, e.g., direct penetration in the membrane, endocytosis-mediated entry, and translocation through the formation of a transitory structure.

CPPs may have an amino acid composition that either contains a high relative abundance of positively charged amino acids such as lysine or arginine or has sequences that contain an alternating pattern of polar/charged amino acids and non-polar, hydrophobic amino acids. These two types of structures are referred to as polycationic or amphipathic, respectively. A third class of CPPs are the hydrophobic peptides, containing only apolar residues, with low net charge or have hydrophobic amino acid groups that are crucial for cellular uptake. Another type of CPPs is the trans-activating transcriptional activator (Tat) from Human Immunodeficiency Virus 1 (HIV-1). Examples of CPPs include to Penetratin, Tat (48-60), Transportan, and (R-AhX-R4) (Ahx refers to aminohexanoyD, Kaposi fibroblast growth factor (FGF) signal peptide sequence, integrin β3 signal peptide sequence, polyarginine peptide Args sequence, Guanine rich-molecular transporters, and sweet arrow peptide. Examples of CPPs and related applications also include those described in U.S. Pat. No. 8,372,951.

CPPs can be used for in vitro and ex vivo work quite readily, and extensive optimization for each cargo and cell type is usually required. In some examples, CPPs may be covalently attached to the Cas protein directly, which is then complexed with the gRNA and delivered to cells. In some examples, separate delivery of CPP-Cas and CPP-gRNA to multiple cells may be performed. CPP may also be used to delivery RNPs.

CPPs may be used to deliver the compositions and systems to plants. In some examples, CPPs may be used to deliver the components to plant protoplasts, which are then regenerated to plant cells and further to plants.

### DNA Nanoclews

In some embodiments, the delivery vehicles comprise DNA nanoclews. A DNA nanoclew refers to a sphere-like structure of DNA (e.g., with a shape of a ball of yarn). The nanoclew may be synthesized by rolling circle amplification with palindromic sequences that aide in the self-assembly of the structure. The sphere may then be loaded with a payload. An example of DNA nanoclew is described in Sun W et al, J Am Chem Soc. 2014 Oct. 22; 136 (42): 14722-5; and Sun W et al, Angew Chem Int Ed Engl. 2015 Oct. 5; 54 (41): 12029-33. DNA nanoclew may have a palindromic sequences to be partially complementary to the gRNA within the Cas: gRNA ribonucleoprotein complex. A DNA nanoclew may be coated, e.g., coated with PEI to induce endosomal escape.

### Gold Nanoparticles

In some embodiments, the delivery vehicles comprise gold nanoparticles (also referred to AuNPs or colloidal gold). Gold nanoparticles may form complex with cargos, e.g., Cas: gRNA RNP. Gold nanoparticles may be coated, e.g., coated in a silicate and an endosomal disruptive polymer, PAsp (DET). Examples of gold nanoparticles include AuraSense Therapeutics' Spherical Nucleic Acid (SNATM) constructs, and those described in Mout R, et al. (2017). ACS Nano 11:2452-8; Lee K, et al. (2017). Nat Biomed Eng 1:889-901.

iTop

In some embodiments, the delivery vehicles comprise iTOP. iTOP refers to a combination of small molecules drives the highly efficient intracellular delivery of native proteins, independent of any transduction peptide. iTOP may be used for induced transduction by osmocytosis and propanebetaine, using NaCl-mediated hyperosmolality together with a transduction compound (propanebetaine) to trigger macropinocytotic uptake into cells of extracellular macromolecules. Examples of iTOP methods and reagents include those described in D′Astolfo D S, Pagliero R J, Pras A, et al. (2015). Cell 161:674-690.

### Polymer-Based Particles

In some embodiments, the delivery vehicles may comprise polymer-based particles (e.g., nanoparticles). In some embodiments, the polymer-based particles may mimic a viral mechanism of membrane fusion. The polymer-based particles may be a synthetic copy of Influenza virus machinery and form transfection complexes with various types of nucleic acids (siRNA, miRNA, plasmid DNA or shRNA, mRNA) that cells take up via the endocytosis pathway, a process that involves the formation of an acidic compartment. The low pH in late endosomes acts as a chemical switch that renders the particle surface hydrophobic and facilitates membrane crossing. Once in the cytosol, the particle releases its payload for cellular action. This Active Endosome Escape technology is safe and maximizes transfection efficiency as it is using a natural uptake pathway. In some embodiments, the polymer-based particles may comprise alkylated and carboxyalkylated branched polyethylenimine. In some examples, the polymer-based particles are VIROMER, e.g., VIROMER RNAi, VIROMER RED, VIROMER mRNA, VIROMER CRISPR. Example methods of delivering the systems and compositions herein include those described in Bawage S S et al., Synthetic mRNA expressed Cas13a mitigates RNA virus infections, www.biorxiv.org/content/10.1101/370460v1.full doi: doi.org/10.1101/370460, Viromer® RED, a powerful tool for transfection of keratinocytes. doi: 10.13140/RG.2.2.16993.61281, Viromer® Transfection-Factbook 2018: technology, product overview, users' data., doi: 10.13140/RG.2.2.23912.16642.

### Streptolysin O (SLO)

The delivery vehicles may be streptolysin O (SLO). SLO is a toxin produced by Group A streptococci that works by creating pores in mammalian cell membranes. SLO may act in a reversible manner, which allows for the delivery of proteins (e.g., up to 100 kDa) to the cytosol of cells without compromising overall viability. Examples of SLO include those described in Sierig G, et al. (2003). Infect Immun 71:446-55; Walev I, et al. (2001). Proc Natl Acad Sci USA 98:3185-90; Teng K W, et al. (2017). Elife 6: e25460.

### Multifunctional Envelope-Type Nanodevice (MEND)

The delivery vehicles may comprise multifunctional envelope-type nanodevice (MENDs). MENDs may comprise condensed plasmid DNA, a PLL core, and a lipid film shell. A MEND may further comprise cell-penetrating peptide (e.g., stearyl octaarginine). The cell penetrating peptide may be in the lipid shell. The lipid envelope may be modified with one or more functional components, e.g., one or more of: polyethylene glycol (e.g., to increase vascular circulation time), ligands for targeting of specific tissues/cells, additional cell-penetrating peptides (e.g., for greater cellular delivery), lipids to enhance endosomal escape, and nuclear delivery tags. In some examples, the MEND may be a tetra-lamellar MEND (T-MEND), which may target the cellular nucleus and mitochondria. In certain examples, a MEND may be a PEG-peptide-DOPE-conjugated MEND (PPD-MEND), which may target bladder cancer cells. Examples of MENDs include those described in Kogure K, et al. (2004). J Control Release 98:317-23; Nakamura T, et al. (2012). Acc Chem Res 45:1113-21.

### Lipid-Coated Mesoporous Silica Particles

The delivery vehicles may comprise lipid-coated mesoporous silica particles. Lipid-coated mesoporous silica particles may comprise a mesoporous silica nanoparticle core and a lipid membrane shell. The silica core may have a large internal surface area, leading to high cargo loading capacities. In some embodiments, pore sizes, pore chemistry, and overall particle sizes may be modified for loading different types of cargos. The lipid coating of the particle may also be modified to maximize cargo loading, increase circulation times, and provide precise targeting and cargo release. Examples of lipid-coated mesoporous silica particles include those described in Du X, et al. (2014). Biomaterials 35:5580-90; Durfee P N, et al. (2016). ACS Nano 10:8325-45.

### Inorganic Nanoparticles

The delivery vehicles may comprise inorganic nanoparticles. Examples of inorganic nanoparticles include carbon nanotubes (CNTs) (e.g., as described in Bates K and Kostarelos K. (2013). Adv Drug Deliv Rev 65:2023-33.), bare mesoporous silica nanoparticles (MSNPs) (e.g., as described in Luo G F, et al. (2014). Sci Rep 4:6064), and dense silica nanoparticles (SiNPs) (as described in Luo D and Saltzman W M. (2000). Nat Biotechnol 18:893-5).

### Exosomes

The delivery vehicles may comprise exosomes. Exosomes include membrane bound extracellular vesicles, which can be used to contain and delivery various types of biomolecules, such as proteins, carbohydrates, lipids, and nucleic acids, and complexes thereof (e.g., RNPs). Examples of exosomes include those described in Schroeder A, et al., J Intern Med. 2010 January;267 (1): 9-21; El-Andaloussi S, et al., Nat Protoc. 2012 December;7 (12): 2112-26; Uno Y, et al., Hum Gene Ther. 2011 June;22 (6): 711-9; Zou W, et al., Hum Gene Ther. 2011 April;22 (4): 465-75.

In some examples, the exosome may form a complex (e.g., by binding directly or indirectly) to one or more components of the cargo. In certain examples, a molecule of an exosome may be fused with first adapter protein and a component of the cargo may be fused with a second adapter protein. The first and the second adapter protein may specifically bind each other, thus associating the cargo with the exosome. Examples of such exosomes include those described in Ye Y, et al., Biomater Sci. 2020 Apr. 28. doi: 10.1039/d0bm00427h.

### Modified Cells and Organisms

Described herein are modified cells, cell populations, and organisms that can be modified by the engineered systems described herein. The modified cells, cell populations, and organisms can have an insertion of one or more polynucleotides, deletion of one or more polynucleotides, mutation of one or more polynucleotides, or a combination thereof. The modification can result in activation of one or more genes, inactivation of one or more genes, modulation of one or more genes, or a combination thereof. Cells, including cells in an organism, can be modified in vitro, in situ, ex vivo, or in vivo. In some embodiments, the modification is insertion or deletion of a polynucleotide, gene, or allele of interest. In some embodiments, the polynucleotide, gene, or allele of interest is associated with a genetic disease or condition.

### Modified Cells

Also described herein are modified cells and cell populations that can be modified by an embodiment of a polynucleotide modifying agent or system described in greater detail elsewhere herein. In some embodiments, a cell is modified the CRISPR-Cas or Cas-based components of the engineered systems described herein. In some embodiments, a cell is modified by the transposase components of the engineered systems described herein. In some embodiments, a cell is modified by the CRISPR-Cas and CRISPR-associated Tn7 transposase components of the engineered systems described herein. In some embodiments, the cell is a eukaryotic cell. In some embodiments, the eukaryotic cell is a mammalian cell. In some embodiments, the eukaryotic cell is a non-human mammalian cell. In some embodiments, the cell is a human cell. In some embodiments, the cell is a plant cell. In some embodiments, the cell is a fungal cell. In some embodiments, the cell is a prokaryotic cell. The cells can be modified in vitro, ex vivo, or in vivo. The cells can be modified by delivering a polynucleotide modifying agent or system described in greater detail elsewhere herein or a component thereof into a cell by a suitable delivery mechanism. Suitable delivery methods and techniques include but are not limited to, transfection via a vector, transduction with viral particles, electroporation, endocytic methods, and others, which are described elsewhere herein and will be appreciated by those of ordinary skill in the art in view of this disclosure.

The modified cells can be further optionally cultured and/or expanded in vitro or ex vivo using any suitable cell culture techniques or conditions, which unless specified otherwise herein, will be appreciated by one of ordinary skill in the art in view of this disclosure. In some embodiments, the cells can be modified, optionally cultured and/or expanded, and administered to a subject in need thereof. In some embodiments, cells can be isolated from a subject, subsequently modified and optionally cultured and/or expanded, and administered back to the subject, such as in a cell therapy. In some embodiments, the cell therapy is an adoptive cell therapy. Such administration can be referred to as autologous administration. In some embodiments, cells can be isolated from a first subject, subsequently modified, optionally cultured and/or expanded, and administered to a second subject, where the first subject and the second subject are different. Such administration can be referred to as non-autologous administration.

In some embodiments, the modified cells can be used as a bioreactor for production of a bioproduct. In some embodiments engineered compositions of the present invention introduce a gene or polynucleotide or otherwise modify the cell to produce one or more bioproducts. In some embodiments, the engineered compositions of the present invention are used to modify a producer cell so as to improve production of a bioproduct. For example, one or more genes and/or transcripts of a cell that limit or decrease efficiency of production of a bioproduct may be modified by the CRISPR-Cas and/or CRISPR-associated Tn7 transposase components of the engineered systems described herein such that efficiency in production of and/or amount of the bioproduct is increased. In some embodiments, one or more genes and/or transcripts of a cell are modified such that they enhance production or efficiency of production of the bioproduct.

### Organisms

Also described herein are modified organisms. In some embodiments, the modified organisms can include one or more modified cells as are described elsewhere herein. In some embodiments, the modified organism is a non-human mammal. In some embodiments, the modified organism is a modified plant. In some embodiments, the modified organism is an insect. In some embodiments, the modified organism is a fungus. In some embodiments, the modified organism is a fungus. The modified organisms can be generated using a that can be modified by an embodiment of the engineered or non-natural guided excision-transposition system described herein. Methods of making modified organisms are described in greater detail elsewhere herein.

The systems and methods described herein can be used in non-animal organisms, e.g., plants, fungi to generated modified non-animal organisms. The system and methods described can be used to generate non-human animal organisms. The system and methods described herein can be used to modify non-germline cells in a human. In some embodiments, the modification is expression of a polynucleotide of interest, gene of interest, and/or allele of interest.

### Non-Human Animals

The systems and methods may be used to generate modified non-human animals and cells thereof. In an aspect, the invention provides a non-human eukaryotic organism; preferably a multicellular eukaryotic organism, comprising a eukaryotic host cell according to any of the described embodiments. In other aspects, the invention provides a eukaryotic organism; preferably a multicellular eukaryotic organism, comprising a eukaryotic host cell according to any of the described embodiments. The organism in some embodiments of these aspects may be an animal, for example, a mammal. Also, the organism may be an arthropod such as an insect. The present invention may also be extended to other agricultural applications such as, for example, farm and production animals. For example, pigs have many features that make them attractive as biomedical models, especially in regenerative medicine. In particular, pigs with severe combined immunodeficiency (SCID) may provide useful models for regenerative medicine, xenotransplantation (discussed also elsewhere herein), and tumor development and will aid in developing therapies for human SCID patients. Lee et al., (Proc Natl Acad Sci USA. 2014 May 20; 111 (20): 7260-5) utilized a reporter-guided transcription activator-like effector nuclease (TALEN) system to generated targeted modifications of recombination activating gene (RAG) 2 in somatic cells at high efficiency, including some that affected both alleles. Such techniques and modifications can be adapted for and used with the modifying agent(s) and systems thereof described herein to generate a modified non-human animal or cell thereof.

The methods of Lee et al., (Proc Natl Acad Sci USA. 2014 May 20; 111 (20): 7260-5) may be applied to the present invention analogously as follows. Mutated pigs are produced by targeted insertion for example in RAG2 in fetal fibroblast cells followed by SCNT and embryo transfer. Constructs coding for CRISPR Cas and a reporter are electroporated into fetal-derived fibroblast cells. After 48 h, transfected cells expressing the green fluorescent protein are sorted into individual wells of a 96-well plate at an estimated dilution of a single cell per well. Targeted modification of RAG2 is screened by amplifying a genomic DNA fragment flanking any CRISPR Cas cutting sites followed by sequencing the PCR products. After screening and ensuring lack of off-site mutations, cells carrying targeted modification of RAG2 are used for SCNT. The polar body, along with a portion of the adjacent cytoplasm of oocyte, presumably containing the metaphase II plate, are removed, and a donor cell are placed in the perivitelline. The reconstructed embryos are then electrically porated to fuse the donor cell with the oocyte and then chemically activated. The activated embryos are incubated in Porcine Zygote Medium 3 (PZM3) with 0.5 μM Scriptaid (S7817; Sigma-Aldrich) for 14-16 h. Embryos are then washed to remove the Scriptaid and cultured in PZM3 until they were transferred into the oviducts of surrogate pigs. Such techniques and modifications can be adapted for and used with the modifying agent(s) and systems thereof described herein to generate a modified non-human animal or cell thereof.

The modified non-human animals described herein can be a platform to model a disease or disorder of an animal, including but not limited to mammals. In some of these embodiments, the mammal can be a human. In certain embodiments, such models and platforms are rodent based, in non-limiting examples rat or mouse. Such models and platforms can take advantage of distinctions among and comparisons between inbred rodent strains. In certain embodiments, such models and platforms primate, horse, cattle, sheep, goat, swine, dog, cat or bird-based, for example to directly model diseases and disorders of such animals or to create modified and/or improved lines of such animals. Advantageously, in certain embodiments, an animal-based platform or model is created to mimic a human disease or disorder. For example, the similarities of swine to humans make swine an ideal platform for modeling human diseases. Compared to rodent models, development of swine models has been costly and time intensive. On the other hand, swine and other animals are much more similar to humans genetically, anatomically, physiologically and pathophysiologically. The present invention provides a high efficiency platform for targeted gene and genome editing, gene and genome modification and gene and genome regulation to be used in such animal platforms and models. Though ethical standards block development of human models and in many cases models based on non-human primates, the present invention is used with in vitro systems, including but not limited to cell culture systems, three dimensional models and systems, and organoids to mimic, model, and investigate genetics, anatomy, physiology and pathophysiology of structures, organs, and systems of humans. The platforms and models provide manipulation of single or multiple targets.

In certain embodiments, the present invention is applicable to disease models like that of Schomberg et al. (FASEB Journal, April 2016; 30 (1): Suppl 571.1). To model the inherited disease neurofibromatosis type 1 (NF-1) Schomberg used CRISPR-Cas9 to introduce mutations in the swine neurofibromin 1 gene by cytosolic microinjection of CRISPR/Cas9 components into swine embryos. CRISPR guide RNAs (gRNA) were created for regions targeting sites both upstream and downstream of an exon within the gene for targeted cleavage by Cas9 and repair was mediated by a specific single-stranded oligodeoxynucleotide (ssODN) template to introduce a 2500 bp deletion. The systems were also used to engineer swine with specific NF-1 mutations or clusters of mutations, and further can be used to engineer mutations that are specific to or representative of a given human individual. Such techniques and modifications can be adapted for and used with the modifying agent(s) and systems thereof described herein to generate a modified non-human animal or cell thereof. In some embodiments, the polynucleotide modifying agent(s) or systems thereof can be similarly used to develop animal models, including but not limited to swine models, of human multigenic diseases. In some embodiments, multiple genetic loci in one gene or in multiple genes are simultaneously targeted using multiplexed guides and optionally one or multiple templates.

SNPs of other animals, such as cows can also be modified or generated using one or more polynucleotide modifying agents or systems described herein. Tan et al. (Proc Natl Acad Sci USA. 2013 Oct. 8; 110 (41): 16526-16531) expanded the livestock gene editing toolbox to include transcription activator-like (TAL) effector nuclease (TALEN)-and clustered regularly interspaced short palindromic repeats (CRISPR)/Cas9-stimulated homology-directed repair (HDR) using plasmid, rAAV, and oligonucleotide templates. Gene specific gRNA sequences were cloned into the Church lab gRNA vector (Addgene ID: 41824) according to their methods (Mali P, et al. (2013) RNA-Guided Human Genome Engineering via Cas9. Science 339 (6121): 823-826). The Cas9 nuclease was provided either by co-transfection of the hCas9 plasmid (Addgene ID: 41815) or mRNA synthesized from RCIScript-hCas9. This RCIScript-hCas9 was constructed by sub-cloning the Xbal-Agel fragment from the hCas9 plasmid (encompassing the hCas9 cDNA) into the RCIScript plasmid. Such techniques and modifications can be adapted for and used with the modifying agent(s) and systems thereof described herein to generate a modified non-human animal or cell thereof.

Heo et al. (Stem Cells Dev. 2015 Feb. 1; 24 (3): 393-402. doi: 10.1089/scd.2014.0278. Epub 2014 Nov. 3) reported highly efficient gene targeting in the bovine genome using bovine pluripotent cells and clustered regularly interspaced short palindromic repeat (CRISPR)/Cas9 nuclease. First, Heo et al. generate induced pluripotent stem cells (iPSCs) from bovine somatic fibroblasts by the ectopic expression of yamanaka factors and GSK3B and MEK inhibitor (2i) treatment. Heo et al. observed that these bovine iPSCs are highly similar to naïve pluripotent stem cells with regard to gene expression and developmental potential in teratomas. Moreover, CRISPR-Cas9 nuclease, which was specific for the bovine NANOG locus, showed highly efficient editing of the bovine genome in bovine iPSCs and embryos. Such techniques and modifications can be adapted for and used with the modifying agent(s) and systems thereof described herein to generate a modified non-human animal or cell thereof.

Igenity® provides a profile analysis of animals, such as cows, to perform and transmit traits of economic traits of economic importance, such as carcass composition, carcass quality, maternal and reproductive traits and average daily gain. The analysis of a comprehensive Igenity® profile begins with the discovery of DNA markers (most often single nucleotide polymorphisms or SNPs). All the markers behind the Igenity® profile were discovered by independent scientists at research institutions, including universities, research organizations, and government entities such as USDA. Markers are then analyzed at Igenity® in validation populations. Igenity® uses multiple resource populations that represent various production environments and biological types, often working with industry partners from the seedstock, cow-calf, feedlot and/or packing segments of the beef industry to collect phenotypes that are not commonly available. Cattle genome databases are widely available, see, e.g., the NAGRP Cattle Genome Coordination Program (www.animalgenome.org/cattle/maps/db.htmD. Thus, the polynucleotide modifying agent(s) and/or systems described herein can be applied to target bovine SNPs. One of skill in the art may utilize the above protocols for targeting SNPs and apply them to bovine SNPs as described, for example, by Tan et al. or Heo et al.

Qingjian Zou et al. (Journal of Molecular Cell Biology Advance Access published Oct. 12, 2015) demonstrated increased muscle mass in dogs by targeting the first exon of the dog Myostatin (MSTN) gene (a negative regulator of skeletal muscle mass). First, the efficiency of the sgRNA was validated, using cotransfection of the sgRNA targeting MSTN with a Cas9 vector into canine embryonic fibroblasts (CEFs). Thereafter, MSTN KO dogs were generated by micro-injecting embryos with normal morphology with a mixture of Cas9 mRNA and MSTN sgRNA and auto-transplantation of the zygotes into the oviduct of the same female dog. The knock-out puppies displayed an obvious muscular phenotype on thighs compared with its wild-type littermate sister. This can also be performed using the polynucleotide agent(s) and/or systems provided herein. Such techniques and modifications can be adapted for and used with the modifying agent(s) and systems thereof described herein to generate a modified non-human animal or cell thereof.

### Livestock

Also described herein are modified pigs or cells that can express one or more polynucleotides, genes or alleles of interest. As reported by Kristin M Whitworth and Dr Randall Prather et al. (Nature Biotech 3434 published online 7 Dec. 2015) CD163 (a viral target) was targeted using CRISPR-Cas9 and the offspring of edited pigs were resistant when exposed to PRRSv. One founder male and one founder female, both of whom had mutations in exon 7 of CD163, were bred to produce offspring. The founder male possessed an 11-bp deletion in exon 7 on one allele, which results in a frameshift mutation and missense translation at amino acid 45 in domain 5 and a subsequent premature stop codon at amino acid 64. The other allele had a 2-bp addition in exon 7 and a 377-bp deletion in the preceding intron, which were predicted to result in the expression of the first 49 amino acids of domain 5, followed by a premature stop code at amino acid 85. The sow had a 7 bp addition in one allele that when translated was predicted to express the first 48 amino acids of domain 5, followed by a premature stop codon at amino acid 70. The sow's other allele was unamplifiable. Selected offspring were predicted to be a null animal (CD163−/−), i.e., a CD163 knock out. Such techniques and modifications can be adapted for and used with the modifying agent(s) and systems thereof described herein to generate a modified pig that can express a polynucleotide of interest. Thus, also described herein are modified pigs their progeny that also express one or more copies of the gene or allele of interest. This may be for livestock, breeding or modelling purposes (i.e., a porcine modeD. Semen comprising the modification (e.g., polynucleotide of interest) is also provided.

### Other Animals

Also described herein are other non-human animals that are modified to express one or more polynucleotides, genes or alleles of interest. Suitable polynucleotide modifying agent(s) and/or system thereof described elsewhere herein can be used to generate other non-human animals such as non-human primates, chickens (reviewed in Sid and Schusser et al 2018. Front. Genet. Doi.org/10.3389/fgene.2018.00456) and other avians (e.g., Scott et al. 2010. ILAR J. 51 (4): 353-361), cattle (Yum et al., 2016. Scientific Reports. 6:27185 and Tait-Burkard et al. 2018. Genome Biology. 19:2014.), sheep and goats (see e.g., Kalds et al., 2019. Front. Genet. Doi.org//10.3389/fgene.2019.00750), horses (see e.g., West and Gill. 2016. J. Equine Vet. Sci. 41:1-6), dogs (see e.g., D. Duan. Nature Biomedical Engineering. 2018. 2:795-796), reptiles (see e.g. Rasys et al. 2019. Cell Reports. 28:2288-2292), fish (including but not limited to zebrafish, see e.g. Datsomor et al. 2019. Scientific Reports. 9:7533, Liu et al. 2019. Front. Cell. Dev. Biol. https://doi.org/10.3389/fcell.2019.00013), insects (see e.g., Kotwica-Rolinska et al. 2019. Front. Physiol. https://doi.org/10.3389/fphys.2019.00891; Gantz and Akbari. 2018. Curr. Opin. Insect. Sci. 28:66-72), rabbits (see e.g., Kawano and Honda. 2017. Methods Mol. Biol. 4630:109-120; Liu et al., 2018. Nature Commun. 9:2717; and Liu et al. 2018. Gene. https://doi.org/10.1016/j.gene.2018.01.044), mice (see e.g., Hall et al. 2018. Curr Protoc Cell Biol. 81 (1): e57), rats (see e.g., Back et al. 2019. Neuron. 102 (1): 105-119), amphibians (see e.g., Nakayama et al. 2013. Genesis. 51 (12): 835-843), nematodes (see e.g., J.B. Lok. 2019. Front. Genet. https://doi.org/10.3389/fgene.2019.00656), molluscs (see e.g., Abe and Kuroda. 2019. Development. 146: dev175976 doi: 10.1242/dev.175976, geckos, shrimp and other crustaceans (see e.g., Gui et al. Genes Genomes Genetics: 6 (11): 3757-3764), oysters (Yu et al. 2019; Mar. Biotechnol (NY) 21 (3): 301-309. doi: 10.1007/s10126-019-09885-y), and sponges (see e.g., Revilla-i-Domingo et al. 2018. Genetics. 210 (2) 435-443), the teachings of which can be adapted for use with one or more of the modifying agent(s) and/or systems described herein to generate the modified non-human animal or cell thereof.

### Therapeutic Applications

Also provided herein are methods of diagnosing, prognosing, treating, and/or preventing a disease, state, or condition in or of a subject. Generally, the methods of diagnosing, prognosing, treating, and/or preventing a disease, state, or condition in or of a subject can include modifying a polynucleotide in a subject or cell thereof using a composition, system, or component thereof described herein and/or include detecting a diseased or healthy polynucleotide in a subject or cell thereof using a composition, system, or component thereof described herein. In some embodiments, the method of treatment or prevention can include using a composition, system, or component thereof to modify a polynucleotide of an infectious organism (e.g., bacterial or virus) within a subject or cell thereof. In some embodiments, the method of treatment or prevention can include using a composition, system, or component thereof to modify a polynucleotide of an infectious organism or symbiotic organism within a subject. The composition, system, and components thereof can be used to develop models of diseases, states, or conditions. The composition, system, and components thereof can be used to detect a disease state or correction thereof, such as by a method of treatment or prevention described herein. The composition, system, and components thereof can be used to screen and select cells that can be used, for example, as treatments or preventions described herein. The composition, system, and components thereof can be used to develop biologically active agents that can be used to modify one or more biologic functions or activities in a subject or a cell thereof.

In general, the method can include delivering a composition, system, and/or component thereof to a subject or cell thereof, or to an infectious or symbiotic organism by a suitable delivery technique and/or composition. Once administered the components can operate as described elsewhere herein to elicit a nucleic acid modification event. In some aspects, the nucleic acid modification event can occur at the genomic, epigenomic, and/or transcriptomic level. DNA and/or RNA cleavage, gene activation, and/or gene deactivation can occur. Additional features, uses, and advantages are described in greater detail below. On the basis of this concept, several variations are appropriate to elicit a genomic locus event, including DNA cleavage, gene activation, or gene deactivation. Using the provided compositions, the person skilled in the art can advantageously and specifically target single or multiple loci with the same or different functional domains to elicit one or more genomic locus events. In addition to treating and/or preventing a disease in a subject, the compositions may be applied in a wide variety of methods for screening in libraries in cells and functional modeling in vivo (e.g., gene activation of lincRNA and identification of function; gain-of-function modeling; loss-of-function modeling; the use the compositions of the invention to establish cell lines and transgenic animals for optimization and screening purposes).

The composition, system, and components thereof described elsewhere herein can be used to treat and/or prevent a disease, such as a genetic and/or epigenetic disease, in a subject. The composition, system, and components thereof described elsewhere herein can be used to treat and/or prevent genetic infectious diseases in a subject, such as bacterial infections, viral infections, fungal infections, parasite infections, and combinations thereof. The composition, system, and components thereof described elsewhere herein can be used to modify the composition or profile of a microbiome in a subject, which can in turn modify the health status of the subject. The composition, system, described herein can be used to modify cells ex vivo, which can then be administered to the subject whereby the modified cells can treat or prevent a disease or symptom thereof. This is also referred to in some contexts as adoptive therapy. The composition, system, described herein can be used to treat mitochondrial diseases, where the mitochondrial disease etiology involves a mutation in the mitochondrial DNA.

Also provided is a method of treating a subject, e.g., a subject in need thereof, comprising inducing gene editing by transforming the subject with the polynucleotide encoding one or more components of the composition, system, or complex or any of polynucleotides or vectors described herein and administering them to the subject. A suitable repair template may also be provided, for example delivered by a vector comprising said repair template. Also provided is a method of treating a subject, e.g., a subject in need thereof, comprising inducing transcriptional activation or repression of multiple target gene loci by transforming the subject with the polynucleotides or vectors described herein, wherein said polynucleotide or vector encodes or comprises one or more components of composition, system, complex or component thereof comprising multiple Cas effectors. Where any treatment is occurring ex vivo, for example in a cell culture, then it will be appreciated that the term ‘subject’ may be replaced by the phrase “cell or cell culture.”

Also provided is a method of treating a subject, e.g., a subject in need thereof, comprising inducing gene editing by transforming the subject with the Cas effector(s), advantageously encoding and expressing in vivo the remaining portions of the composition, system, (e.g., RNA, guides). A suitable repair template may also be provided, for example delivered by a vector comprising said repair template. Also provided is a method of treating a subject, e.g., a subject in need thereof, comprising inducing transcriptional activation or repression by transforming the subject with the Cas effector(s) advantageously encoding and expressing in vivo the remaining portions of the composition, system, (e.g., RNA, guides); advantageously in some embodiments the CRISPR enzyme is a catalytically inactive Cas effector and includes one or more associated functional domains. Where any treatment is occurring ex vivo, for example in a cell culture, then it will be appreciated that the term ‘subject’ may be replaced by the phrase “cell or cell culture.”

One or more components of the composition and system described herein can be included in a composition, such as a pharmaceutical composition, and administered to a host individually or collectively. Alternatively, these components may be provided in a single composition for administration to a host. Administration to a host may be performed via viral vectors known to the skilled person or described herein for delivery to a host (e.g., lentiviral vector, adenoviral vector, AAV vector). As explained herein, use of different selection markers (e.g., for lentiviral gRNA selection) and concentration of gRNA (e.g., dependent on whether multiple gRNAs are used) may be advantageous for eliciting an improved effect.

Thus, also described herein are methods of inducing one or more polynucleotide modifications in a eukaryotic or prokaryotic cell or component thereof (e.g., a mitochondria) of a subject, infectious organism, and/or organism of the microbiome of the subject. The modification can include the introduction, deletion, or substitution of one or more nucleotides at a target sequence of a polynucleotide of one or more cell(s). The modification can occur in vitro, ex vivo, in situ, or in vivo.

In some embodiments, the method of treating or inhibiting a condition or a disease caused by one or more mutations in a genomic locus in a eukaryotic organism or a non-human organism can include manipulation of a target sequence within a coding, non-coding or regulatory element of said genomic locus in a target sequence in a subject or a non-human subject in need thereof comprising modifying the subject or a non-human subject by manipulation of the target sequence and wherein the condition or disease is susceptible to treatment or inhibition by manipulation of the target sequence including providing treatment comprising delivering a composition comprising the particle delivery system or the delivery system or the virus particle of any one of the above embodiment or the cell of any one of the above embodiment.

Also provided herein is the use of the particle delivery system or the delivery system or the virus particle of any one of the above embodiments or the cell of any one of the above embodiments in ex vivo or in vivo gene or genome editing; or for use in in vitro, ex vivo or in vivo gene therapy. Also provided herein are particle delivery systems, non-viral delivery systems, and/or the virus particle of any one of the above embodiments or the cell of any one of the above embodiments used in the manufacture of a medicament for in vitro, ex vivo or in vivo gene or genome editing or for use in in vitro, ex vivo or in vivo gene therapy or for use in a method of modifying an organism or a non-human organism by manipulation of a target sequence in a genomic locus associated with a disease or in a method of treating or inhibiting a condition or disease caused by one or more mutations in a genomic locus in a eukaryotic organism or a non-human organism.

In some embodiments, polynucleotide modification can include the introduction, deletion, or substitution of 1-75 nucleotides at each target sequence of said polynucleotide of said cell(s). The modification can include the introduction, deletion, or substitution of at least 1, 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence. The modification can include the introduction, deletion, or substitution of at least 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s). The modification can include the introduction, deletion, or substitution of at least 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s). The modification can include the introduction, deletion, or substitution of at least 20, 21, 22, 23, 24, 25, 26, 27, 28, 29, 30, 35, 40, 45, 50, or 75 nucleotides at each target sequence of said cell(s). The modification can include the introduction, deletion, or substitution of at least 40, 45, 50, 75, 100, 200, 300, 400 or 500 nucleotides at each target sequence of said cell(s). The modification can include the introduction, deletion, or substitution of at least 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, or 9900 to 10000 nucleotides at each target sequence of said cell(s).

In some embodiments, the modifications can include the introduction, deletion, or substitution of nucleotides at each target sequence of said cell(s) via nucleic acid components (e.g., guide(s) RNA(s) or sgRNA(s)), such as those mediated by a composition, system, or a component thereof described elsewhere herein. In some embodiments, the modifications can include the introduction, deletion, or substitution of nucleotides at a target or random sequence of said cell(s) via a composition, system, or technique.

The target sequences of polynucleotides to be modified to treat or prevent disease are described in greater detail below.

As is also discussed elsewhere herein, the composition, system, can include a template polynucleotide (also referred to herein as template nucleic acids or template sequence). In an embodiment, the template nucleic acid alters the structure of the target position by participating in homologous recombination. In an embodiment, the template nucleic acid alters the sequence of the target position. In an embodiment, the template nucleic acid results in the incorporation of a modified, or non-naturally occurring base into the target nucleic acid.

The template sequence may undergo a breakage mediated or catalyzed recombination with the target sequence. In an embodiment, the template nucleic acid can include sequence that corresponds to a site on the target sequence that is cleaved, nicked, or otherwise modified by one or more Cas effector mediated cleavage event(s). In an embodiment, the template nucleic acid can include sequence that corresponds to both, a first site on the target sequence that is cleaved, nicked, or otherwise modified in a first Cas effector mediated event, and a second site on the target sequence that is cleaved in a second Cas effector mediated event.

In certain embodiments, the template nucleic acid can include a sequence which results in an alteration in the coding sequence of a translated sequence, e.g., one which results in the substitution of one amino acid for another in a protein product, e.g., transforming a mutant allele into a wild type allele, transforming a wild type allele into a mutant allele, and/or introducing a stop codon, insertion of an amino acid residue, deletion of an amino acid residue, or a nonsense mutation. In certain embodiments, the template nucleic acid can include sequence which results in an alteration in a non-coding sequence, e.g., an alteration in an exon or in a 5′ or 3′ non-translated or non-transcribed region. Such alterations include an alteration in a control element, e.g., a promoter, enhancer, and an alteration in a cis-acting or trans-acting control element.

A template nucleic acid having homology with a target position in a target gene may be used to alter the structure of a target sequence. The template sequence may be used to alter an unwanted structure, e.g., an unwanted or mutant nucleotide. The template nucleic acid may include sequence which, when integrated, results in: decreasing the activity of a positive control element; increasing the activity of a positive control element; decreasing the activity of a negative control element; increasing the activity of a negative control element; decreasing the expression of a gene; increasing the expression of a gene; increasing resistance to a disorder or disease; increasing resistance to viral entry; correcting a mutation or altering an unwanted amino acid residue conferring, increasing, abolishing or decreasing a biological property of a gene product, e.g., increasing the enzymatic activity of an enzyme, or increasing the ability of a gene product to interact with another molecule.

The template nucleic acid may include sequence which results in: a change in sequence of 1, 2, 3, 4, 5, 6, 7, 8, 9, 10, 11, 12 or more nucleotides of the target sequence. In an embodiment, the template nucleic acid may be 20+/−10, 30+/−10, 40+/−10, 50+/−10, 60+/−10, 70+/−10, 80 +/−10, 90+/−10, 100+/−10, 110+/−10, 120+/−10, 130+/−10, 140+/−10, 150+/−10, 160+/−10, 170+/−10, 180+/−10, 190+/−10, 200+/−10, 210+/−10, or 220+/−10 nucleotides in length. In an embodiment, the template nucleic acid may be 30+/−20, 40+/−20, 50+/−20, 60+/−20, 70+/−20, 80+/−20, 90+/−20, 100+/−20, 110+/−20, 120+/−20, 130+/−20, 140+/−20, 150+/−20, 160+/−20, 170+/−20, 180+/−20, 190+/−20, 200+/−20, 210+/−20, or 220+/−20 nucleotides in length. In an embodiment, the template nucleic acid is 10 to 1,000, 20 to 900, 30 to 800, 40 to 700, 50 to 600, 50 to 500, 50 to 400, 50 to 300, 50 to 200, or 50 to 100 nucleotides in length.

A template nucleic acid comprises the following components: [5′ homology arm]-[replacement sequence]-[3′ homology arm]. The homology arms provide for recombination into the chromosome, thus replacing the undesired element, e.g., a mutation or signature, with the replacement sequence. In an embodiment, the homology arms flank the most distal cleavage sites. In an embodiment, the 3′ end of the 5′ homology arm is the position next to the 5′ end of the replacement sequence. In an embodiment, the 5′ homology arm can extend at least 10, 20, 30, 40, 50, 100, 200, 300, 400, 500, 600, 700, 800, 900, 1000, 1500, or 2000 nucleotides 5′ from the 5′ end of the replacement sequence. In an embodiment, the 5′ end of the 3′ homology arm is the position next to the 3′ end of the replacement sequence. In an embodiment, the 3′ homology arm can extend at least 10, 20, 30, 40, 50, 100, 200, 300, 400, 500, 600, 700, 800, 900, 1000, 1500, or 2000 nucleotides 3′ from the 3′ end of the replacement sequence.

In certain embodiments, one or both homology arms may be shortened to avoid including certain sequence repeat elements. For example, a 5′ homology arm may be shortened to avoid a sequence repeat element. In other embodiments, a 3′ homology arm may be shortened to avoid a sequence repeat element. In some embodiments, both the 5′ and the 3′ homology arms may be shortened to avoid including certain sequence repeat elements.

In certain embodiments, template nucleic acids for correcting a mutation may designed for use as a single-stranded oligonucleotide. When using a single-stranded oligonucleotide, 5′ and 3′ homology arms may range up to about 200 base pairs (bp) in length, e.g., at least 25, 50, 75, 100, 125, 150, 175, or 200 bp in length.

In some embodiments, the composition, system, or component thereof can promote Non-Homologous End-Joining (NHEJ). In some embodiments, modification of a polynucleotide by a composition, system, or a component thereof, such as a diseased polynucleotide, can include NHEJ. In some embodiments, promotion of this repair pathway by the composition, system, or a component thereof can be used to target gene or polynucleotide specific knock-outs and/or knock-ins. In some embodiments, promotion of this repair pathway by the composition, system, or a component thereof can be used to generate NHEJ-mediated indels. Nuclease-induced NHEJ can also be used to remove (e.g., delete) sequence in a gene of interest. Generally, NHEJ repairs a double-strand break in the DNA by joining together the two ends; however, generally, the original sequence is restored only if two compatible ends, exactly as they were formed by the double-strand break, are perfectly ligated. The DNA ends of the double-strand break are frequently the subject of enzymatic processing, resulting in the addition or removal of nucleotides, at one or both strands, prior to rejoining of the ends. This results in the presence of insertion and/or deletion (indeD mutations in the DNA sequence at the site of the NHEJ repair. The indel can range in size from 1-50 or more base pairs. In some embodiments thee indel can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 base pairs or more. If a double-strand break is targeted near to a short target sequence, the deletion mutations caused by the NHEJ repair often span, and therefore remove, the unwanted nucleotides. For the deletion of larger DNA segments, introducing two double-strand breaks, one on each side of the sequence, can result in NHEJ between the ends with removal of the entire intervening sequence. Both of these approaches can be used to delete specific DNA sequences.

In some embodiments, composition, system, mediated NHEJ can be used in the method to delete small sequence motifs. In some embodiments, composition, system, mediated NHEJ can be used in the method to generate NHEJ-mediate indels that can be targeted to the gene, e.g., a coding region, e.g., an early coding region of a gene of interest can be used to knockout (i.e., eliminate expression of) a gene of interest. For example, early coding region of a gene of interest includes sequence immediately following a transcription start site, within a first exon of the coding sequence, or within 500 bp of the transcription start site (e.g., less than 500, 450, 400, 350, 300, 250, 200, 150, 100 or 50 bp). In an embodiment, in which a guide RNA and Cas effector generate a double strand break for the purpose of inducing NHEJ-mediated indels, a guide RNA may be configured to position one double-strand break in close proximity to a nucleotide of the target position. In an embodiment, the cleavage site may be between 0-500 bp away from the target position (e.g., less than 500, 400, 300, 200, 100, 50, 40, 30, 25, 20, 15, 10, 9, 8, 7, 6, 5, 4, 3, 2 or 1 bp from the target position). In an embodiment, in which two guide RNAs complexing with one or more Cas nickases induce two single strand breaks for the purpose of inducing NHEJ-mediated indels, two guide RNAs may be configured to position two single-strand breaks to provide for NHEJ repair a nucleotide of the target position.

For minimization of toxicity and off-target effect, it may be important to control the concentration of Cas mRNA and guide RNA delivered. Optimal concentrations of Cas mRNA and guide RNA can be determined by testing different concentrations in a cellular or non-human eukaryote animal model and using deep sequencing the analyze the extent of modification at potential off-target genomic loci. Alternatively, to minimize the level of toxicity and off-target effect, Cas nickase mRNA (for example S. pyogenes Cas9 with the D10A mutation) can be delivered with a pair of guide RNAs targeting a site of interest. Guide sequences and strategies to minimize toxicity and off-target effects can be as in WO 2014/093622 (PCT/US2013/074667); or, via mutation. Others are as described elsewhere herein.

Typically, in the context of an endogenous CRISPR or CAST system, formation of a CRISPR or CAST complex (comprising a guide sequence hybridized to a target sequence and complexed with one or more Cas proteins) results in cleavage, nicking, and/or another modification of one or both strands in or near (e.g., within 1, 2, 3, 4, 5, 6, 7, 8, 9, 10, 20, 50, or more base pairs from) the target sequence. In some embodiments, the tracr sequence, which may comprise or consist of all or a portion of a wild-type tracr sequence (e.g., about or more than about 20, 26, 32, 45, 48, 54, 63, 67, 85, or more nucleotides of a wild-type tracr sequence), can also form part of a CRISPR complex, such as by hybridization along at least a portion of the tracr sequence to all or a portion of a tracr mate sequence that is operably linked to the guide sequence.

In some embodiments, a method of modifying a target polynucleotide in a cell to treat or prevent a disease can include allowing a composition, system, or component thereof to bind to the target polynucleotide, e.g., to effect cleavage, nicking, or other modification as the composition, system, is capable of said target polynucleotide, thereby modifying the target polynucleotide, wherein the composition, system, or component thereof, complex with a guide sequence, and hybridize said guide sequence to a target sequence within the target polynucleotide, wherein said guide sequence is optionally linked to a tracr mate sequence, which in turn can hybridize to a tracr sequence. In some of these embodiments, the composition, system, or component thereof can be or include a CRISPR-Cas effector complexed with a guide sequence. In some embodiments, modification can include cleaving or nicking one or two strands at the location of the target sequence by one or more components of the composition, system, or component thereof.

The cleavage, nicking, or other modification capable of being performed by the composition, system, can modify transcription of a target polynucleotide. In some embodiments, modification of transcription can include decreasing transcription of a target polynucleotide. In some embodiments, modification can include increasing transcription of a target polynucleotide. In some embodiments, the method includes repairing said cleaved target polynucleotide by homologous recombination with an exogenous template polynucleotide, wherein said repair results in a modification such as, but not limited to, an insertion, deletion, or substitution of one or more nucleotides of said target polynucleotide. In some embodiments, said modification results in one or more amino acid changes in a protein expressed from a gene comprising the target sequence. In some embodiments, the modification imparted by the composition, system, or component thereof provides a transcript and/or protein that can correct a disease or a symptom thereof, including but not limited to, any of those described in greater detail elsewhere herein.

In some embodiments, the method of treating or preventing a disease can include delivering one or more vectors or vector systems to a cell, such as a eukaryotic or prokaryotic cell, wherein one or more vectors or vector systems include the composition, system, or component thereof. In some embodiments, the vector(s) or vector system(s) can be a viral vector or vector system, such as an AAV or lentiviral vector system, which are described in greater detail elsewhere herein. In some embodiments, the method of treating or preventing a disease can include delivering one or more viral particles, such as an AAV or lentiviral particle, containing the composition, system, or component thereof. In some embodiments, the viral particle has a tissue specific tropism. In some embodiments, the viral particle has a liver, muscle, eye, heart, pancreas, kidney, neuron, epithelial cell, endothelial cell, astrocyte, glial cell, immune cell, or red blood cell specific tropism.

It will be understood that the composition, system, according to the invention as described herein, such as the composition, system, for use in the methods according to the invention as described herein, may be suitably used for any type of application known for composition, system, preferably in eukaryotes. In certain aspects, the application is therapeutic, preferably therapeutic in a eukaryote organism, such as including but not limited to animals (including human), plants, algae, fungi (including yeasts), etc. Alternatively, or in addition, in certain aspects, the application may involve accomplishing or inducing one or more particular traits or characteristics, such as genotypic and/or phenotypic traits or characteristics, as also described elsewhere herein.

### Treating Diseases of the Circulatory System

In some embodiments, the composition, system, and/or component thereof described herein can be used to treat and/or prevent a circulatory system disease. Exemplary disease is provided, for example, in Tables 3 and 4. In some embodiments the plasma exosomes of Wahlgren et al. (Nucleic Acids Research, 2012, Vol. 40, No. 17e130) can be used to deliver the composition, system, and/or component thereof described herein to the blood. In some embodiments, the circulatory system disease can be treated by using a lentivirus to deliver the composition, system, described herein to modify hematopoietic stem cells (HSCs) in vivo or ex vivo (see e.g., Drakopoulou, “Review Article, The Ongoing Challenge of Hematopoietic Stem Cell-Based Gene Therapy for β-Thalassemia,” Stem Cells International, Volume 2011, Article ID 987980, 10 pages, doi: 10.4061/2011/987980, which can be adapted for use with the composition, system, herein in view of the description herein). In some embodiments, the circulatory system disorder can be treated by correcting HSCs as to the disease using a composition, system, herein or a component thereof, wherein the composition, system, optionally includes a suitable HDR repair template (see e.g., Cavazzana, “Outcomes of Gene Therapy for β-Thalassemia Major via Transplantation of Autologous Hematopoietic Stem Cells Transduced Ex Vivo with a Lentiviral BA-T87Q-Globin Vector.”; Cavazzana-Calvo, “Transfusion independence and HMGA2 activation after gene therapy of human B-thalassaemia”, Nature 467, 318-322 (16 Sep. 2010) doi: 10.1038/nature09328; Nienhuis, “Development of Gene Therapy for Thalassemia, Cold Spring Harbor Perspectives in Medicine, doi: 10.1101/cshperspect.a011833 (2012), LentiGlobin BB305, a lentiviral vector containing an engineered B-globin gene (BA-T87Q; and Xie et al., “Seamless gene correction of B-thalassaemia mutations in patient-specific iPSCs using CRISPR/Cas9 and piggyback” Genome Research gr. 173427.114 (2014) http://www.genome.org/cgi/doi/10.1101/gr.173427.114 (Cold Spring Harbor Laboratory Press; Watts, “Hematopoietic Stem Cell Expansion and Gene Therapy” Cytotherapy 13 (10): 1164-1171. doi: 10.3109/14653249.2011.620748 (2011), which can be adapted for use with the composition, system, herein in view of the description herein). In some embodiments, iPSCs can be modified using a composition, system, described herein to correct a disease polynucleotide associated with a circulatory disease. In this regard, the teachings of Xu et al. (Sci Rep. 2015 Jul. 9; 5:12065. doi: 10.1038/srep12065) and Song et al. (Stem Cells Dev. 2015 May 1; 24 (9): 1053-65. doi: 10.1089/scd.2014.0347. Epub 2015 Feb. 5) with respect to modifying iPSCs can be adapted for use in view of the description herein with the composition, system, described herein.

The term “Hematopoietic Stem Cell” or “HSC” refers broadly those cells considered to be an HSC, e.g., blood cells that give rise to all the other blood cells and are derived from mesoderm; located in the red bone marrow, which is contained in the core of most bones. HSCs of the invention include cells having a phenotype of hematopoietic stem cells, identified by small size, lack of lineage (lin) markers, and markers that belong to the cluster of differentiation series, like: CD34, CD38, CD90, CD133, CD105, CD45, and also c-kit,-the receptor for stem cell factor. Hematopoietic stem cells are negative for the markers that are used for detection of lineage commitment, and are, thus, called Lin-; and, during their purification by FACS, a number of up to 14 different mature blood-lineage markers, e.g., CD13 & CD33 for myeloid, CD71 for erythroid, CD19 for B cells, CD61 for megakaryocytic, etc. for humans; and, B220 (murine CD45) for B cells, Mac-1 (CD11b/CD18) for monocytes, Gr-1 for Granulocytes, Ter119 for erythroid cells, I17Ra, CD3, CD4, CD5, CD8 for T cells, etc. Mouse HSC markers: CD34lo/−, SCA-1+, Thy 1.1+/lo, CD38+, C-kit+, lin−, and Human HSC markers: CD34+, CD59+, Thyl/CD90+, CD38lo/−, C-kit/CD117+, and lin−. HSCs are identified by markers. Hence in embodiments discussed herein, the HSCs can be CD34+ cells. HSCs can also be hematopoietic stem cells that are CD34-/CD38-. Stem cells that may lack c-kit on the cell surface that are considered in the art as HSCs are within the ambit of the invention, as well as CD133+ cells likewise considered HSCs in the art.

The CRISPR-Cas system may be engineered to target genetic locus or loci in HSCs. In some embodiments, the Cas effector(s) can be codon-optimized for a eukaryotic cell and especially a mammalian cell, e.g., a human cell, for instance, HSC, or iPSC and sgRNA targeting a locus or loci in HSC, such as circulatory disease, can be prepared. These may be delivered via particles. The particles may be formed by the Cas effector (e.g., Cas9) protein and the gRNA being admixed. The gRNA and Cas effector (e.g., Cas9) protein mixture can be, for example, admixed with a mixture comprising or consisting essentially of or consisting of surfactant, phospholipid, biodegradable polymer, lipoprotein and alcohol, whereby particles containing the gRNA and Cas effector (e.g., Cas9) protein may be formed. The invention comprehends so making particles and particles from such a method as well as uses thereof. Particles suitable delivery of the CRISRP-Cas systems in the context of blood or circulatory system or HSC delivery to the blood or circulatory system are described in greater detail elsewhere herein.

In some embodiments, after ex vivo modification the HSCs or iPCS can be expanded prior to administration to the subject. Expansion of HSCs can be via any suitable method such as that described by, Lee, “Improved ex vivo expansion of adult hematopoietic stem cells by overcoming CUL4-mediated degradation of HOXB4.” Blood. 2013 May 16; 121 (20): 4082-9. doi: 10.1182/blood-2012-09-455204. Epub 2013 Mar. 21.

In some embodiments, the HSCs or iPSCs modified can be autologous. In some embodiments, the HSCs or iPSCs can be allogenic. In addition to the modification of the disease gene(s), allogenic cells can be further modified using the composition, system, described herein to reduce the immunogenicity of the cells when delivered to the recipient. Such techniques are described elsewhere herein and e.g., Cartier, “MINI-SYMPOSIUM: X-Linked Adrenoleukodystrophypa, Hematopoietic Stem Cell Transplantation and Hematopoietic Stem Cell Gene Therapy in X-Linked Adrenoleukodystrophy,” Brain Pathology 20 (2010) 857-862, which can be adapted for use with the composition, system, herein.

### Treating Diseases of the Brain

In some embodiments, the compositions, systems, described herein can be used to treat diseases of the brain and CNS. Delivery options for the brain include encapsulation of CRISPR enzyme and guide RNA in the form of either DNA or RNA into liposomes and conjugating to molecular Trojan horses for trans-blood brain barrier (BBB) delivery. Molecular Trojan horses have been shown to be effective for delivery of B-gal expression vectors into the brain of non-human primates. The same approach can be used to delivery vectors containing CRISPR enzyme and guide RNA. For instance, Xia C F and Boado R J, Pardridge W M (“Antibody-mediated targeting of siRNA via the human insulin receptor using avidin-biotin technology.” Mol Pharm. 2009 May-Jun;6 (3): 747-51. doi: 10.1021/mp800194) describes how delivery of short interfering RNA (siRNA) to cells in culture, and in vivo, is possible with combined use of a receptor-specific monoclonal antibody (mAb) and avidin-biotin technology. The authors also report that because the bond between the targeting mAb and the siRNA is stable with avidin-biotin technology, and RNAi effects at distant sites such as brain are observed in vivo following an intravenous administration of the targeted siRNA, the teachings of which can be adapted for use with the compositions, systems, herein. In other embodiments, an artificial virus can be generated for CNS and/or brain delivery. See e.g., Zhang et al. (Mol Ther. 2003 January;7 (1): 11-8.), the teachings of which can be adapted for use with the compositions, systems, herein.

### Treating Hearing Diseases

In some embodiments the composition, system, described herein can be used to treat a hearing disease or hearing loss in one or both ears. Deafness is often caused by lost or damaged hair cells that cannot relay signals to auditory neurons. In such cases, cochlear implants may be used to respond to sound and transmit electrical signals to the nerve cells. But these neurons often degenerate and retract from the cochlea as fewer growth factors are released by impaired hair cells.

In some embodiments, the composition, system, or modified cells can be delivered to one or both ears for treating or preventing hearing disease or loss by any suitable method or technique. Suitable methods and techniques include, but are not limited to, those set forth in U.S. patent application No. 20120328580 describes injection of a pharmaceutical composition into the ear (e.g., auricular administration), such as into the luminae of the cochlea (e.g., the Scala media, Sc vestibulae, and Sc tympani), e.g., using a syringe, e.g., a single-dose syringe. For example, one or more of the compounds described herein can be administered by intratympanic injection (e.g., into the middle ear), and/or injections into the outer, middle, and/or inner ear; administration in situ, via a catheter or pump (see e.g., Mckenna et al., (U.S. Publication No. 2006/0030837) and Jacobsen et al., (U.S. Pat. No. 7,206,639); administration in combination with a mechanical device such as a cochlear implant or a hearing aid, which is worn in the outer ear (see e.g., U.S. Publication No. 2007/0093878, which provides an exemplary cochlear implant suitable for delivery of the compositions, systems, described herein to the ear). Such methods are routinely used in the art, for example, for the administration of steroids and antibiotics into human ears. Injection can be, for example, through the round window of the ear or through the cochlear capsule. Other inner ear administration methods are known in the art (see, e.g., Salt and Plontke, Drug Discovery Today, 10:1299-1306, 2005). In some embodiments, a catheter or pump can be positioned, e.g., in the ear (e.g., the outer, middle, and/or inner ear) of a patient during a surgical procedure. In some embodiments, a catheter or pump can be positioned, e.g., in the ear (e.g., the outer, middle, and/or inner ear) of a patient without the need for a surgical procedure.

In general, the cell therapy methods described in U.S. patent application No. 20120328580 can be used to promote complete or partial differentiation of a cell to or towards a mature cell type of the inner ear (e.g., a hair celD in vitro. Cells resulting from such methods can then be transplanted or implanted into a patient in need of such treatment. The cell culture methods required to practice these methods, including methods for identifying and selecting suitable cell types, methods for promoting complete or partial differentiation of selected cells, methods for identifying complete or partially differentiated cell types, and methods for implanting complete or partially differentiated cells are described below.

Cells suitable for use in the present invention include, but are not limited to, cells that are capable of differentiating completely or partially into a mature cell of the inner ear, e.g., a hair cell (e.g., an inner and/or outer hair celD, when contacted, e.g., in vitro, with one or more of the compounds described herein. Exemplary cells that are capable of differentiating into a hair cell include, but are not limited to stem cells (e.g., inner ear stem cells, adult stem cells, bone marrow derived stem cells, embryonic stem cells, mesenchymal stem cells, skin stem cells, iPS cells, and fat derived stem cells), progenitor cells (e.g., inner ear progenitor cells), support cells (e.g., Deiters' cells, pillar cells, inner phalangeal cells, tectal cells and Hensen's cells), and/or germ cells. The use of stem cells for the replacement of inner ear sensory cells is described in Li et al., (U.S. Publication No. 2005/0287127) and Li et al., (U.S. patent Ser. No. 11/953,797). The use of bone marrow derived stem cells for the replacement of inner ear sensory cells is described in Edge et al., PCT/US2007/084654. iPS cells are described, e.g., at Takahashi et al., Cell, Volume 131, Issue 5, Pages 861-872 (2007); Takahashi and Yamanaka, Cell 126, 663-76 (2006); Okita et al., Nature 448, 260-262 (2007); Yu, J. et al., Science 318 (5858): 1917-1920 (2007); Nakagawa et al., Nat. Biotechnol. 26:101-106 (2008); and Zaehres and Scholer, Cell 131 (5): 834-835 (2007). Such suitable cells can be identified by analyzing (e.g., qualitatively or quantitatively) the presence of one or more tissue specific genes. For example, gene expression can be detected by detecting the protein product of one or more tissue-specific genes. Protein detection techniques involve staining proteins (e.g., using cell extracts or whole cells) using antibodies against the appropriate antigen. In this case, the appropriate antigen is the protein product of the tissue-specific gene expression. Although, in principle, a first antibody (i.e., the antibody that binds the antigen) can be labeled, it is more common (and improves the visualization) to use a second antibody directed against the first (e.g., an anti-IgG). This second antibody is conjugated either with fluorochromes, or appropriate enzymes for colorimetric reactions, or gold beads (for electron microscopy), or with the biotin-avidin system, so that the location of the primary antibody, and thus the antigen, can be recognized.

The composition and system may be delivered to the ear by direct application of pharmaceutical composition to the outer ear, with compositions modified from US Published application, 20110142917. In some embodiments the pharmaceutical composition is applied to the ear canal. Delivery to the ear may also be referred to as aural or otic delivery.

In some embodiments, the compositions, systems, or components thereof and/or vectors or vector systems can be delivered to ear via a transfection to the inner ear through the intact round window by a novel proteidic delivery technology which may be applied to the nucleic acid-targeting system of the present invention (see, e.g., Qi et al., Gene Therapy (2013), 1-9). About 40 μl of 10 mM RNA may be contemplated as the dosage for administration to the ear.

According to Rejali et al. (Hear Res. 2007 June;228 (1-2): 180-7), cochlear implant function can be improved by good preservation of the spiral ganglion neurons, which are the target of electrical stimulation by the implant and brain derived neurotrophic factor (BDNF) has previously been shown to enhance spiral ganglion survival in experimentally deafened ears. Rejali et al. tested a modified design of the cochlear implant electrode that includes a coating of fibroblast cells transduced by a viral vector with a BDNF gene insert. To accomplish this type of ex vivo gene transfer, Rejali et al. transduced guinea pig fibroblasts with an adenovirus with a BDNF gene cassette insert, and determined that these cells secreted BDNF and then attached BDNF-secreting cells to the cochlear implant electrode via an agarose gel, and implanted the electrode in the scala tympani. Rejali et al. determined that the BDNF expressing electrodes were able to preserve significantly more spiral ganglion neurons in the basal turns of the cochlea after 48 days of implantation when compared to control electrodes and demonstrated the feasibility of combining cochlear implant therapy with ex vivo gene transfer for enhancing spiral ganglion neuron survival. Such a system may be applied to the nucleic acid-targeting system of the present invention for delivery to the ear.

In some embodiments, the system set forth in Mukherjea et al. (Antioxidants & Redox Signaling, Volume 13, Number 5, 2010) can be adapted for transtympanic administration of the composition, system, or component thereof to the ear. In some embodiments, a dosage of about 2 mg to about 4 mg of CRISPR Cas for administration to a human.

In some embodiments, the system set forth in Jung et al. (Molecular Therapy, vol. 21 no. 4, 834-841 Apr. 2013) can be adapted for vestibular epithelial delivery of the composition, system, or component thereof to the ear. In some embodiments, a dosage of about 1 to about 30 mg of CRISPR Cas for administration to a human.

### Treating Diseases in Non-Dividing Cells

In some embodiments, the gene or transcript to be corrected is in a non-dividing cell. Exemplary non-dividing cells are muscle cells or neurons. Non-dividing (especially non-dividing, fully differentiated) cell types present issues for gene targeting or genome engineering, for example because homologous recombination (HR) is generally suppressed in the G1 cell-cycle phase. However, while studying the mechanisms by which cells control normal DNA repair systems, Durocher discovered a previously unknown switch that keeps HR “off” in non-dividing cells and devised a strategy to toggle this switch back on. Orthwein et al. (Daniel Durocher's lab at the Mount Sinai Hospital in Ottawa, Canada) recently reported (Nature 16142, published online 9 Dec. 2015) have shown that the suppression of HR can be lifted and gene targeting successfully concluded in both kidney (293T) and osteosarcoma (U2OS) cells. Tumor suppressors, BRCA1, PALB2 and BRAC2 are known to promote DNA DSB repair by HR. They found that formation of a complex of BRCA1 with PALB2-BRAC2 is governed by a ubiquitin site on PALB2, such that action on the site by an E3 ubiquitin ligase. This E3 ubiquitin ligase is composed of KEAP1 (a PALB2-interacting protein) in complex with cullin −3 (CUL3)-RBX1. PALB2 ubiquitylation suppresses its interaction with BRCA1 and is counteracted by the deubiquitylase USP11, which is itself under cell cycle control. Restoration of the BRCA1-PALB2 interaction combined with the activation of DNA-end resection is sufficient to induce homologous recombination in G1, as measured by a number of methods including a CRISPR-Cas9-based gene-targeting assay directed at USP11 or KEAP1 (expressed from a pX459 vector). However, when the BRCA1-PALB2 interaction was restored in resection-competent G1 cells using either KEAP1 depletion or expression of the PALB2-KR mutant, a robust increase in gene-targeting events was detected. These teachings can be adapted for and/or applied to the Cas compositions, systems, described herein.

Thus, reactivation of HR in cells, especially non-dividing, fully differentiated cell types is preferred, in some embodiments. In some embodiments, promotion of the BRCA1-PALB2 interaction is preferred in some embodiments. In some embodiments, the target cell is a non-dividing cell. In some embodiments, the target cell is a neuron or muscle cell. In some embodiments, the target cell is targeted in vivo. In some embodiments, the cell is in G1 and HR is suppressed. In some embodiments, use of KEAP1 depletion, for example inhibition of expression of KEAP1 activity, is preferred. KEAP1 depletion may be achieved through siRNA, for example as shown in Orthwein et al. Alternatively, expression of the PALB2-KR mutant (lacking all eight Lys residues in the BRCA1-interaction domain is preferred, either in combination with KEAP1 depletion or alone. PALB2-KR interacts with BRCA1 irrespective of cell cycle position. Thus, promotion or restoration of the BRCA1-PALB2 interaction, especially in G1 cells, is preferred in some embodiments, especially where the target cells are non-dividing, or where removal and return (ex vivo gene targeting) is problematic, for example neuron or muscle cells. KEAP1 siRNA is available from ThermoFischer. In some embodiments, a BRCA1-PALB2 complex may be delivered to the G1 cell. In some embodiments, PALB2 deubiquitylation may be promoted for example by increased expression of the deubiquitylase USP11, so it is envisaged that a construct may be provided to promote or up-regulate expression or activity of the deubiquitylase USP11.

### Treating Diseases of the Eye

In some embodiments, the disease to be treated is a disease that affects the eyes. Thus, in some embodiments, the composition, system, or component thereof described herein is delivered to one or both eyes.

The composition, system, can be used to correct ocular defects that arise from several genetic mutations further described in Genetic Diseases of the Eye, Second Edition, edited by Elias I. Traboulsi, Oxford University Press, 2012.

In some embodiments, the condition to be treated or targeted is an eye disorder. In some embodiments, the eye disorder may include glaucoma. In some embodiments, the eye disorder includes a retinal degenerative disease. In some embodiments, the retinal degenerative disease is selected from Stargardt disease, Bardet-Biedl Syndrome, Best disease, Blue Cone Monochromacy, Choroidermia, Cone-rod dystrophy, Congenital Stationary Night Blindness, Enhanced S-Cone Syndrome, Juvenile X-Linked Retinoschisis, Leber Congenital Amaurosis, Malattia Leventinesse, Norrie Disease or X-linked Familial Exudative Vitreoretinopathy, Pattern Dystrophy, Sorsby Dystrophy, Usher Syndrome, Retinitis Pigmentosa, Achromatopsia or Macular dystrophies or degeneration, Retinitis Pigmentosa, Achromatopsia, and age related macular degeneration. In some embodiments, the retinal degenerative disease is Leber Congenital Amaurosis (LCA) or Retinitis Pigmentosa. Other exemplary eye diseases are described in greater detail elsewhere herein.

In some embodiments, the composition, system, is delivered to the eye, optionally via intravitreal injection or subretinal injection. Intraocular injections may be performed with the aid of an operating microscope. For subretinal and intravitreal injections, eyes may be prolapsed by gentle digital pressure and fundi visualized using a contact lens system consisting of a drop of a coupling medium solution on the cornea covered with a glass microscope slide coverslip. For subretinal injections, the tip of a 10-mm 34-gauge needle, mounted on a 5-μl Hamilton syringe may be advanced under direct visualization through the superior equatorial sclera tangentially towards the posterior pole until the aperture of the needle was visible in the subretinal space. Then, 2 μl of vector suspension may be injected to produce a superior bullous retinal detachment, thus confirming subretinal vector administration. This approach creates a self-sealing sclerotomy allowing the vector suspension to be retained in the subretinal space until it is absorbed by the RPE, usually within 48 h of the procedure. This procedure may be repeated in the inferior hemisphere to produce an inferior retinal detachment. This technique results in the exposure of approximately 70% of neurosensory retina and RPE to the vector suspension. For intravitreal injections, the needle tip may be advanced through the sclera 1 mm posterior to the corneoscleral limbus and 2 μl of vector suspension injected into the vitreous cavity. For intracameral injections, the needle tip may be advanced through a corneoscleral limbal paracentesis, directed towards the central cornea, and 2 μl of vector suspension may be injected. For intracameral injections, the needle tip may be advanced through a corneoscleral limbal paracentesis, directed towards the central cornea, and 2 μl of vector suspension may be injected. These vectors may be injected at titers of either 1.0-1.4×10¹⁰ or 1.0-1.4×10⁹ transducing units (TU)/ml.

In some embodiments, for administration to the eye, lentiviral vectors. In some embodiments, the lentiviral vector is an equine infectious anemia virus (EIAV) vector. Exemplary EIAV vectors for eye delivery are described in Balagaan, J Gene Med 2006; 8:275-285, Published online 21 Nov. 2005 in Wiley InterScience (www.interscience.wiley.com). DOI: 10.1002/jgm.845; Binley et al., HUMAN GENE THERAPY 23:980-991 (September 2012), which can be adapted for use with the composition, system, described herein. In some embodiments, the dosage can be 1.1×10⁵ transducing units per eye (TU/eye) in a total volume of 100 μl.

Other viral vectors can also be used for delivery to the eye, such as AAV vectors, such as those described in Campochiaro et al., Human Gene Therapy 17:167-176 (February 2006), Millington-Ward et al. (Molecular Therapy, vol. 19 no. 4, 642-649 Apr. 2011; Dalkara et al. (Sci Transl Med 5, 189ra76 (2013)), which can be adapted for use with the composition, system, described herein. In some embodiments, the dose can range from about 106 to 109.5 particle units. In the context of the Millington-Ward AAV vectors, a dose of about 2×10¹¹ to about 6×10¹³ virus particles can be administered. In the context of Dalkara vectors, a dose of about 1×10¹⁵ to about 1×10¹⁶ vg/ml administered to a human.

In some embodiments, the sd-rxRNA® system of RXi Pharmaceuticals may be used/and or adapted for delivering composition, system, to the eye. In this system, a single intravitreal administration of 3 μg of sd-rxRNA results in sequence-specific reduction of PPIB mRNA levels for 14 days. The sd-rxRNA® system may be applied to the nucleic acid-targeting system of the present invention, contemplating a dose of about 3 to 20 mg of CRISPR administered to a human.

In other embodiments, the methods of US Patent Publication No. 20130183282, which is directed to methods of cleaving a target sequence from the human rhodopsin gene, may also be modified to the nucleic acid-targeting system of the present invention.

In other embodiments, the methods of US Patent Publication No. 20130202678 for treating retinopathies and sight-threatening ophthalmologic disorders relating to delivering of the Puf-A gene (which is expressed in retinal ganglion and pigmented cells of eye tissues and displays a unique anti-apoptotic activity) to the sub-retinal or intravitreal space in the eye. In particular, desirable targets are zgc: 193933, prdm1a, spata2, tex10, rbb4, ddx3, zp2.2, Blimp-1 and HtrA2, all of which may be targeted by the composition, system, of the present invention.

Wu (Cell Stem Cell, 13:659-62, 2013) designed a guide RNA that led Cas9 to a single base pair mutation that causes cataracts in mice, where it induced DNA cleavage. Then using either the other wild-type allele or oligos given to the zygotes repair mechanisms corrected the sequence of the broken allele and corrected the cataract-causing genetic defect in mutant mouse. This approach can be adapted to and/or applied to the compositions, systems, described herein.

US Patent Publication No. 20120159653, describes use of zinc finger nucleases to genetically modify cells, animals and proteins associated with macular degeneration (MD), the teachings of which can be applied to and/or adapted for the compositions, systems, described herein.

One aspect of US Patent Publication No. 20120159653 relates to editing of any chromosomal sequences that encode proteins associated with MD which may be applied to the nucleic acid-targeting system of the present invention.

### Treating Muscle Diseases and Cardiovascular Diseases

In some embodiments, the composition, system, can be used to treat and/or prevent a muscle disease and associated circulatory or cardiovascular disease or disorder. The present invention also contemplates delivering the composition, system, described herein, e.g. Cas effector protein systems, to the heart. For the heart, a myocardium tropic adeno-associated virus (AAVM) is preferred, in particular AAVM41 which showed preferential gene transfer in the heart (see, e.g., Lin-Yanga et al., PNAS, Mar. 10, 2009, vol. 106, no. 10). Administration may be systemic or local. A dosage of about 1-10×10¹⁴ vector genomes is contemplated for systemic administration. See also, e.g., Eulalio et al. (2012) Nature 492:376 and Somasuntharam et al. (2013) Biomaterials 34:7790, the teachings of which can be adapted for and/or applied to the compositions, systems, described herein.

For example, US Patent Publication No. 20110023139, the teachings of which can be adapted for and/or applied to the compositions, systems, described herein describes use of zinc finger nucleases to genetically modify cells, animals and proteins associated with cardiovascular disease. Cardiovascular diseases generally include high blood pressure, heart attacks, heart failure, and stroke and TIA. Any chromosomal sequence involved in cardiovascular disease or the protein encoded by any chromosomal sequence involved in cardiovascular disease may be utilized in the methods described in this disclosure. The cardiovascular-related proteins are typically selected based on an experimental association of the cardiovascular-related protein to the development of cardiovascular disease. For example, the production rate or circulating concentration of a cardiovascular-related protein may be elevated or depressed in a population having a cardiovascular disorder relative to a population lacking the cardiovascular disorder. Differences in protein levels may be assessed using proteomic techniques including but not limited to Western blot, immunohistochemical staining, enzyme linked immunosorbent assay (ELISA), and mass spectrometry. Alternatively, the cardiovascular-related proteins may be identified by obtaining gene expression profiles of the genes encoding the proteins using genomic techniques including but not limited to DNA microarray analysis, serial analysis of gene expression (SAGE), and quantitative real-time polymerase chain reaction (Q-PCR). Exemplary chromosomal sequences can be found in Table 3.

The compositions, systems, herein can be used for treating diseases of the muscular system. The present invention also contemplates delivering the composition, system, described herein, e.g., Cas (e.g. Cas9 and/or Cas12) effector protein systems, to muscle(s).

In some embodiments, the muscle disease to be treated is a muscle dystrophy such as DMD. In some embodiments, the composition, system, such as a system capable of RNA modification, described herein can be used to achieve exon skipping to achieve correction of the diseased gene. As used herein, the term “exon skipping” refers to the modification of pre-mRNA splicing by the targeting of splice donor and/or acceptor sites within a pre-mRNA with one or more complementary antisense oligonucleotide(s) (AONs). By blocking access of a spliceosome to one or more splice donor or acceptor site, an AON may prevent a splicing reaction thereby causing the deletion of one or more exons from a fully-processed mRNA. Exon skipping may be achieved in the nucleus during the maturation process of pre-mRNAs. In some examples, exon skipping may include the masking of key sequences involved in the splicing of targeted exons by using a composition, system, described herein capable of RNA modification. In some embodiments, exon skipping can be achieved in dystrophin mRNA. In some embodiments, the composition, system, can induce exon skipping at exon 1, 2, 3, 4, 5, 6, 7, 8, 10, 11, 12, 13, 14, 15, 16, 17, 18, 19, 20, 21, 22, 23, 24, 25, 26, 27, 28, 29, 30, 31, 32, 33, 34, 45, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or any combination thereof of the dystrophin mRNA. In some embodiments, the composition, system, can induce exon skipping at exon 43, 44, 50, 51, 52, 55, or any combination thereof of the dystrophin mRNA. Mutations in these exons, can also be corrected using non-exon skipping polynucleotide modification methods.

In some embodiments, for treatment of a muscle disease, the method of Bortolanza et al. Molecular Therapy vol. 19 no. 11, 2055-264 Nov. 2011) may be applied to an AAV expressing CRISPR Cas and injected into humans at a dosage of about 2× 1015 or 2× 1016 vg of vector. The teachings of Bortolanza et al., can be adapted for and/or applied to the compositions, systems, described herein.

In some embodiments, the method of Dumonceaux et al. (Molecular Therapy vol. 18 no. 5, 881-887 May 2010) may be applied to an AAV expressing CRISPR Cas and injected into humans, for example, at a dosage of about 1014 to about 1015 vg of vector. The teachings of Dumonceaux described herein can be adapted for and/or applied to the compositions, systems, described herein.

In some embodiments, the method of Kinouchi et al. (Gene Therapy (2008) 15, 1126-1130) may be applied to CRISPR Cas systems described herein and injected into a human, for example, at a dosage of about 500 to 1000 ml of a 40 μM solution into the muscle.

In some embodiments, the method of Hagstrom et al. (Molecular Therapy Vol. 10, No. 2, August 2004) can be adapted for and/or applied to the compositions, systems, herein and injected at a dose of about 15 to about 50 mg into the great saphenous vein of a human.

### Treating Diseases of the Liver and Kidney

In some embodiments, the composition, system, or component thereof described herein can be used to treat a disease of the kidney or liver. Thus, in some embodiments, delivery of the CRISRP-Cas system or component thereof described herein is to the liver or kidney.

Delivery strategies to induce cellular uptake of the therapeutic nucleic acid include physical force or vector systems such as viral-, lipid- or complex-based delivery, or nanocarriers. From the initial applications with less possible clinical relevance, when nucleic acids were addressed to renal cells with hydrodynamic high-pressure injection systemically, a wide range of gene therapeutic viral and non-viral carriers have been applied already to target posttranscriptional events in different animal kidney disease models in vivo (Csaba Révész and Péter Hamar (2011). Delivery Methods to Target RNAs in the Kidney, Gene Therapy Applications, Prof. Chunsheng Kang (Ed.), ISBN: 978-953-307-541-9, InTech, Available from: www.intechopen.com/books/gene-therapy-applications/delivery-methods-to-target-rnas-inthe-kidney). Delivery methods to the kidney may include those in Yuan et al. (Am J Physiol Renal Physiol 295: F605-F617, 2008). The method of Yuang et al. may be applied to the CRISPR Cas system of the present invention contemplating a 1-2 g subcutaneous injection of CRISPR Cas conjugated with cholesterol to a human for delivery to the kidneys. In some embodiments, the method of Molitoris et al. (J Am Soc Nephrol 20:1754-1764, 2009) can be adapted to the CRISRP-Cas system of the present invention and a cumulative dose of 12-20 mg/kg to a human can be used for delivery to the proximal tubule cells of the kidneys. In some embodiments, the methods of Thompson et al. (Nucleic Acid Therapeutics, Volume 22, Number 4, 2012) can be adapted to the CRISRP-Cas system of the present invention and a dose of up to 25 mg/kg can be delivered via i.v. administration. In some embodiments, the method of Shimizu et al. (J Am Soc Nephrol 21:622-633, 2010) can be adapted to the CRISRP-Cas system of the present invention and a dose of about of 10-20 μmol CRISPR Cas complexed with nanocarriers in about 1-2 liters of a physiologic fluid for i.p. administration can be used.

Other various delivery vehicles can be used to deliver the composition, system, to the kidney such as viral, hydrodynamic, lipid, polymer nanoparticles, aptamers and various combinations thereof (see e.g. Larson et al., Surgery, (August 2007), Vol. 142, No. 2, pp. (262-269); Hamar et al., Proc Natl Acad Sci, (October 2004), Vol. 101, No. 41, pp. (14883-14888); Zheng et al., Am J Pathol, (October 2008), Vol. 173, No. 4, pp. (973-980); Feng et al., Transplantation, (May 2009), Vol. 87, No. 9, pp. (1283-1289); Q. Zhang et al., PloS ONE, (July 2010), Vol. 5, No. 7, e11709, pp. (1-13); Kushibikia et al., J Controlled Release, (July 2005), Vol. 105, No. 3, pp. (318-331); Wang et al., Gene Therapy, (July 2006), Vol. 13, No. 14, pp. (1097-1103); Kobayashi et al., Journal of Pharmacology and Experimental Therapeutics, (February 2004), Vol. 308, No. 2, pp. (688-693); Wolfrum et al., Nature Biotechnology, (September 2007), Vol. 25, No. 10, pp. (1149-1157); Molitoris et al., J Am Soc Nephrol, (August 2009), Vol. 20, No. 8 pp. (1754-1764); Mikhaylova et al., Cancer Gene Therapy, (March 2011), Vol. 16, No. 3, pp. (217-226); Y. Zhang et al., J Am Soc Nephrol, (April 2006), Vol. 17, No. 4, pp. (1090-1101); Singhal et al., Cancer Res, (May 2009), Vol. 69, No. 10, pp. (4244-4251); Malek et al., Toxicology and Applied Pharmacology, (April 2009), Vol. 236, No. 1, pp. (97-108); Shimizu et al., J Am Soc Nephrology, (April 2010), Vol. 21, No. 4, pp. (622-633); Jiang et al., Molecular Pharmaceutics, (May-June 2009), Vol. 6, No. 3, pp. (727-737); Cao et al, J Controlled Release, (June 2010), Vol. 144, No. 2, pp. (203-212); Ninichuk et al., Am J Pathol, (March 2008), Vol. 172, No. 3, pp. (628-637); Purschke et al., Proc Natl Acad Sci, (March 2006), Vol. 103, No. 13, pp. (5173-5178).

In some embodiments, delivery is to liver cells. In some embodiments, the liver cell is a hepatocyte. Delivery of the composition and system herein may be via viral vectors, especially AAV (and in particular AAV2/6) vectors. These can be administered by intravenous injection. A preferred target for the liver, whether in vitro or in vivo, is the albumin gene. This is a so-called ‘safe harbor” as albumin is expressed at very high levels and so some reduction in the production of albumin following successful gene editing is tolerated. It is also preferred as the high levels of expression seen from the albumin promoter/enhancer allows for useful levels of correct or transgene production (from the inserted donor template) to be achieved even if only a small fraction of hepatocytes are edited. See sites identified by Wechsler et al. (reported at the 57th Annual Meeting and Exposition of the American Society of Hematology-abstract available online at https://ash.confex.com/ash/2015/webprogram/Paper86495.html and presented on 6th December 2015) which can be adapted for use with the compositions, systems, herein.

Exemplary liver and kidney diseases that can be treated and/or prevented are described elsewhere herein.

### Treating Epithelial and Lung Diseases

In some embodiments, the disease treated or prevented by the composition, system, described herein can be a lung or epithelial disease. The compositions, systems, described herein can be used for treating epithelial and/or lung diseases. The present invention also contemplates delivering the composition, system, described herein, to one or both lungs.

In some embodiments, as viral vector can be used to deliver the composition, system, or component thereof to the lungs. In some embodiments, the AAV is an AAV-1, AAV-2, AAV-5, AAV-6, and/or AAV-9 for delivery to the lungs. (see, e.g., Li et al., Molecular Therapy, vol. 17 no. 12, 2067-277 Dec. 2009). In some embodiments, the MOI can vary from 1×10³ to 4×10⁵ vector genomes/cell. In some embodiments, the delivery vector can be an RSV vector as in Zamora et al. (Am J Respir Crit Care Med Vol 183. pp 531-538, 2011. The method of Zamora et al. may be applied to the nucleic acid-targeting system of the present invention and an aerosolized CRISPR Cas, for example with a dosage of 0.6 mg/kg, may be contemplated for the present invention.

Subjects treated for a lung disease may for example receive pharmaceutically effective amount of aerosolized AAV vector system per lung endobronchially delivered while spontaneously breathing. As such, aerosolized delivery is preferred for AAV delivery in general.

An adenovirus or an AAV particle may be used for delivery. Suitable gene constructs, each operably linked to one or more regulatory sequences, may be cloned into the delivery vector. In this instance, the following constructs are provided as examples: Cbh or EF1α promoter for Cas (Cas (e.g., Cas9 and/or Cas12)), U6 or H1 promoter for guide RNA). A preferred arrangement is to use a CFTRdelta508 targeting guide, a repair template for deltaF508 mutation and a codon optimized Cas (e.g. Cas9 and/or Cas12) enzyme, with optionally one or more nuclear localization signal or sequence(s) (NLS(s)), e.g., two (2) NLSs.

### Treating Diseases of the Skin

The compositions, systems, described herein can be used for the treatment of skin diseases. The present invention also contemplates delivering the composition, system, described herein, to the skin.

In some embodiments, delivery to the skin (intradermal delivery) of the composition, system, or component thereof can be via one or more microneedles or microneedle containing device. For example, in some embodiments the device and methods of Hickerson et al. (Molecular Therapy-Nucleic Acids (2013) 2, e129) can be used and/or adapted to deliver the composition, system, described herein, for example, at a dosage of up to 300 μl of 0.1 mg/ml CRISPR-Cas (e.g. Cas9 and/or Cas12) system to the skin.

In some embodiments, the methods and techniques of Leachman et al. (Molecular Therapy, vol. 18 no. 2, 442-446 Feb. 2010) can be used and/or adapted for delivery of a CIRPSR-Cas system described herein to the skin.

In some embodiments, the methods and techniques of Zheng et al. (PNAS, Jul. 24, 2012, vol. 109, no. 30, 11975-11980) can be used and/or adapted for nanoparticle delivery of a CIRPSR-Cas system described herein to the skin. In some embodiments, as dosage of about 25 nM applied in a single application can achieve gene knockdown in the skin.

### Treating Cancer

The compositions, systems, described herein can be used for the treatment of cancer. The present invention also contemplates delivering the composition, system, described herein, to a cancer cell. Also, as is described elsewhere herein the compositions, systems, can be used to modify an immune cell, such as a CAR or CAR T cell, which can then in turn be used to treat and/or prevent cancer. This is also described in WO2015161276, the disclosure of which is hereby incorporated by reference and described herein below.

Target genes suitable for the treatment or prophylaxis of cancer can include those set forth in Tables 4 and 5. In some embodiments, target genes for cancer treatment and prevention can also include those described in WO2015048577 the disclosure of which is hereby incorporated by reference and can be adapted for and/or applied to the composition, system, described herein.

### Diseases

Genetic Diseases and Diseases with a Genetic and/or Epigenetic Aspect

The compositions, systems, or components thereof can be used to treat and/or prevent a genetic disease or a disease with a genetic and/or epigenetic aspect. The genes and conditions exemplified herein are not exhaustive. In some embodiments, a method of treating and/or preventing a genetic disease can include administering a composition, system, and/or one or more components thereof to a subject, where the composition, system, and/or one or more components thereof is capable of modifying one or more copies of one or more genes associated with the genetic disease or a disease with a genetic and/or epigenetic aspect in one or more cells of the subject. In some embodiments, modifying one or more copies of one or more genes associated with a genetic disease or a disease with a genetic and/or epigenetic aspect in the subject can eliminate a genetic disease or a symptom thereof in the subject. In some embodiments, modifying one or more copies of one or more genes associated with a genetic disease or a disease with a genetic and/or epigenetic aspect in the subject can decrease the severity of a genetic disease or a symptom thereof in the subject. In some embodiments, the compositions, systems, or components thereof can modify one or more genes or polynucleotides associated with one or more diseases, including genetic diseases and/or those having a genetic aspect and/or epigenetic aspect, including but not limited to, any one or more set forth in Table 3. It will be appreciated that those diseases and associated genes listed herein are non-exhaustive and non-limiting. Further some genes play roles in the development of multiple diseases.

TABLE 3
Table 3. Exemplary Genetic and Other Diseases and Associated Genes

| | Primary | Additional | |
|---|---|---|---|
| | Tissues or | Tissues/ | |
| | System | Systems | |
| Disease Name | Affected | Affected | Genes |
| Achondroplasia | Bone and | | fibroblast growth factor receptor 3 |
| | Muscle | | (FGFR3) |
| Achromatopsia | eye | | CNGA3, CNGB3, GNAT2, PDE6C, |
| | | | PDE6H, ACHM2, ACHM3, |
| Acute Renal Injury | kidney | | NFkappaB, AATF, p85alpha, FAS, |
| | | | Apoptosis cascade elements (e.g. |
| | | | FASR, Caspase 2, 3, 4, 6, 7, 8, 9, 10, |
| | | | AKT, TNF alpha, IGF1, IGF1R, |
| | | | RIPK1), p53 |
| Age Related Macular | eye | | Abcr; CCL2; CC2; CP |
| Degeneration | | | (ceruloplasmin); Timp3; cathepsinD; |
| | | | VLDLR, CCR2 |
| AIDS | Immune System | | KIR3DL1, NKAT3, NKB1, AMB11, |
| | | | KIR3DS1, IFNG, CXCL12, SDF1 |
| Albinism (including | Skin, hair, eyes, | | TYR, OCA2, TYRP1, and SLC45A2, |
| oculocutaneous albinism (types | | | SLC24A5 and C10orf11 |
| 1-7) and ocular albinism) | | | |
| Alkaptonuria | Metabolism of | Tissues/organs | HGD |
| | amino acids | where | |
| | | homogentisic | |
| | | acid | |
| | | accumulates, | |
| | | particularly | |
| | | cartilage (joints), | |
| | | heart valves, | |
| | | kidneys | |
| alpha-1 antitrypsin deficiency | Lung | Liver, skin, | SERPINA1, those set forth in |
| (AATD or A1AD) | | vascular system, | WO2017165862, PiZ allele |
| | | kidneys, GI | |
| ALS | CNS | | SOD1; ALS2; ALS3; ALS5; |
| | | | ALS7; STEX; FUS; TARDBP; VEGF |
| | | | (VEGF-a; |
| | | | VEGF-b; VEGF-c); DPP6; NEFH, |
| | | | PTGS1, SLC1A2, TNFRSF10B, |
| | | | PRPH, HSP90AA1, CRIA2, IFNG, |
| | | | AMPA2 S100B, FGF2, AOX1, CS, |
| | | | TXN, RAPHJ1, MAP3K5, NBEAL1, |
| | | | GPX1, ICA1L, RAC1, MAPT, ITPR2, |
| | | | ALS2CR4, GLS, ALS2CR8, CNTFR, |
| | | | ALS2CR11, FOLH1, FAM117B, |
| | | | P4HB, CNTF, SQSTM1, STRADB, |
| | | | NAIP, NLR, YWHAQ, SLC33A1, |
| | | | TRAK2, SCA1, NIF3L1, NIF3, |
| | | | PARD3B, COX8A, CDK15, HECW1, |
| | | | HECT, C2, WW 15, NOS1, MET, |
| | | | SOD2, HSPB1, NEFL, CTSB, ANG, |
| | | | HSPA8, RNase A, VAPB, VAMP, |
| | | | SNCA, alpha HGF, CAT, ACTB, |
| | | | NEFM, TH, BCL2, FAS, CASP3, |
| | | | CLU, SMN1, G6PD, BAX, HSF1, |
| | | | RNF19A, JUN, ALS2CR12, HSPA5, |
| | | | MAPK14, APEX1, TXNRD1, NOS2, |
| | | | TIMP1, CASP9, XIAP, GLG1, EPO, |
| | | | VEGFA, ELN, GDNF, NFE2L2, |
| | | | SLC6A3, HSPA4, APOE, PSMB8, |
| | | | DCTN2, TIMP3, KIFAP3, SLC1A1, |
| | | | SMN2, CCNC, STUB1, ALS2, |
| | | | PRDX6, SYP, CABIN1, CASP1, |
| | | | GART, CDK5, ATXN3, RTN4, |
| | | | C1QB, VEGFC, HTT, PARK7, XDH, |
| | | | GFAP, MAP2, CYCS, FCGR3B, CCS, |
| | | | UBL5, MMP9m SLC18A3, TRPM7, |
| | | | HSPB2, AKT1, DEERL1, CCL2, |
| | | | NGRN, GSR, TPPP3, APAF1, |
| | | | BTBD10, GLUD1, CXCR4, S:C1A3, |
| | | | FLT1, PON1, AR, LIF, ERBB3, :GA:S1, |
| | | | CD44, TP53, TLR3, GRIA1, |
| | | | GAPDH, AMPA, GRIK1, DES, |
| | | | CHAT, FLT4, CHMP2B, BAG1, |
| | | | CHRNA4, GSS, BAK1, KDR, GSTP1, |
| | | | OGG1, IL6 |
| Alzheimer's Disease | Brain | | E1; CHIP; UCH; UBB; Tau; LRP; |
| | | | PICALM; CLU; PS1; |
| | | | SORL1; CR1; VLDLR; UBA1; |
| | | | UBA3; CHIP28; AQP1; UCHL1; |
| | | | UCHL3; APP, AAA, CVAP, AD1, |
| | | | APOE, AD2, DCP1, ACE1, MPO, |
| | | | PACIP1, PAXIP1L, PTIP, A2M, |
| | | | BLMH, BMH, PSEN1, AD3, ALAS2, |
| | | | ABCA1, BIN1, BDNF, BTNL8, |
| | | | C1ORF49, CDH4, CHRNB2, |
| | | | CKLFSF2, CLEC4E, CR1L, CSF3R, |
| | | | CST3, CYP2C, DAPK1, ESR1, |
| | | | FCAR, FCGR3B, FFA2, FGA, GAB2, |
| | | | GALP, GAPDHS, GMPB, HP, HTR7, |
| | | | IDE, IF127, IFI6, IFIT2, IL1RN, IL- |
| | | | 1RA, IL8RA, IL8RB, JAG1, KCNJ15, |
| | | | LRP6, MAPT, MARK4, MPHOSPH1, |
| | | | MTHFR, NBN, NCSTN, NIACR2, |
| | | | NMNAT3, NTM, ORM1, P2RY13, |
| | | | PBEF1, PCK1, PICALM, PLAU, |
| | | | PLXNC1, PRNP, PSEN1, PSEN2, |
| | | | PTPRA, RALGPS2, RGSL2, |
| | | | SELENBP1, SLC25A37, SORL1, |
| | | | Mitoferrin-1, TF, TFAM, TNF, |
| | | | TNFRSF10C, UBE1C |
| Amyloidosis | | | APOA1, APP, AAA, CVAP, AD1, |
| | | | GSN, FGA, LYZ, TTR, PALB |
| Amyloid neuropathy | | | TTR, PALB |
| Anemia | Blood | | CDAN1, CDA1, RPS19, DBA, PKLR, |
| | | | PK1, NT5C3, UMPH1, PSN1, RHAG, |
| | | | RH50A, NRAMP2, SPTB, ALAS2, |
| | | | ANH1, ASB, ABCB7, ABC7, ASAT |
| Angelman Syndrome | Nervous system, | | UBE3A |
| | brain | | |
| Attention Deficit Hyperactivity | Brain | | PTCHD1 |
| Disorder (ADHD) | | | |
| Autoimmune lymphoproliferative | Immune system | | TNFRSF6, APT1, FAS, CD95, |
| syndrome | | | ALPS1A |
| Autism, Autism spectrum | Brain | | PTCHD1; Mecp2; BZRAP1; MDGA2; |
| disorders (ASDs), including | | | Sema5A; Neurexin 1; GLO1, RTT, |
| Asperger's and a general | | | PPMX, MRX16, RX79, NLGN3, |
| diagnostic category called | | | NLGN4, KIAA1260, AUTSX2, |
| Pervasive Developmental | | | FMR1, FMR2; FXR1; FXR2; |
| Disorders (PDDs) | | | MGLUR5, ATP10C, CDH10, GRM6, |
| | | | MGLUR6, CDH9, CNTN4, NLGN2, |
| | | | CNTNAP2, SEMA5A, DHCR7, |
| | | | NLGN4X, NLGN4Y, DPP6, NLGN5, |
| | | | EN2, NRCAM, MDGA2, NRXN1, |
| | | | FMR2, AFF2, FOXP2, OR4M2, |
| | | | OXTR, FXR1, FXR2, PAH, |
| | | | GABRA1, PTEN, GABRA5, PTPRZ1, |
| | | | GABRB3, GABRG1, HIRIP3, |
| | | | SEZ6L2, HOXA1, SHANK3, IL6, |
| | | | SHBZRAP1, LAMB1, SLC6A4, |
| | | | SERT, MAPK3, TAS2R1, MAZ, |
| | | | TSC1, MDGA2, TSC2, MECP2, |
| | | | UBE3A, WNT2, see also |
| | | | 20110023145 |
| autosomal dominant polycystic | kidney | liver | PKD1, PKD2 |
| kidney disease (ADPKD) - | | | |
| (includes diseases such as von | | | |
| Hippel-Lindau disease and | | | |
| tubreous sclerosis complex | | | |
| disease) | | | |
| Autosomal Recessive Polycystic | kidney | liver | PKDH1 |
| Kidney Disease (ARPKD) | | | |
| Ataxia-Telangiectasia (a.k.a | Nervous system, | various | ATM |
| Louis Bar syndrome) | immune system | | |
| B-Cell Non-Hodgkin Lymphoma | | | BCL7A, BCL7 |
| Bardet-Biedl syndrome | Eye, | Liver, ear, | ARL6, BBS1, BBS2, BBS4, BBS5, |
| | musculoskeletal | gastrointestinal | BBS7, BBS9, BBS10, BBS12, |
| | system, kidney, | system, brain | CEP290, INPP5E, LZTFL1, MKKS, |
| | reproductive | | MKS1, SDCCAG8, TRIM32, TTC8 |
| | organs | | |
| Bare Lymphocyte Syndrome | blood | | TAPBP, TPSN, TAP2, ABCB3, PSF2, |
| | | | RING11, MHC2TA, C2TA, RFX5, |
| | | | RFXAP, RFX5 |
| Barter's Syndrome (types I, II, | kidney | | SLC12A1 (type I), KCNJ1 (type II), |
| III, IVA and B, and V) | | | CLCNKB (type III), BSND (type IV |
| | | | A), or both the CLCNKA CLCNKB |
| | | | genes (type IV B), CASR (type V). |
| Becker muscular dystrophy | Muscle | | DMD, BMD, MYF6 |
| Best Disease (Vitelliform | eye | | VMD2 |
| Macular Dystrophy type 2) | | | |
| Bleeding Disorders | blood | | TBXA2R, P2RX1, P2X1 |
| Blue Cone Monochromacy | eye | | OPN1LW, OPN1MW, and LCR |
| Breast Cancer | Breast tissue | | BRCA1, BRCA2, COX-2 |
| Bruton's Disease (aka X-linked | Immune system, | | BTK |
| Agammglobulinemia) | specifically B | | |
| | cells | | |
| Cancers (e.g., lymphoma, chronic | Various | | FAS, BID, CTLA4, PDCD1, CBLB, |
| lymphocytic leukemia (CLL), B | | | PTPN6, TRAC, TRBC, those |
| cell acute lymphocytic leukemia | | | described in WO2015048577 |
| (B-ALL), acute lymphoblastic | | | |
| leukemia, acute myeloid | | | |
| leukemia, non-Hodgkin's | | | |
| lymphoma (NHL), diffuse large | | | |
| cell lymphoma (DLCL), multiple | | | |
| myeloma, renal cell carcinoma | | | |
| (RCC), neuroblastoma, colorectal | | | |
| cancer, breast cancer, ovarian | | | |
| cancer, melanoma, sarcoma, | | | |
| prostate cancer, lung cancer, | | | |
| esophageal cancer, hepatocellular | | | |
| carcinoma, pancreatic cancer, | | | |
| astrocytoma, mesothelioma, head | | | |
| and neck cancer, and | | | |
| medulloblastoma | | | |
| Cardiovascular Diseases | heart | Vascular system | IL1B, XDH, TP53, PTGS, MB, IL4, |
| | | | ANGPT1, ABCGu8, CTSK, PTGIR, |
| | | | KCNJ11, INS, CRP, PDGFRB, |
| | | | CCNA2, PDGFB, KCNJ5, KCNN3, |
| | | | CAPN10, ADRA2B, ABCG5, |
| | | | PRDX2, CPAN5, PARP14, MEX3C, |
| | | | ACE, RNF, IL6, TNF, STN, |
| | | | SERPINE1, ALB, ADIPOQ, APOB, |
| | | | APOE, LEP, MTHFR, APOA1, |
| | | | EDN1, NPPB, NOS3, PPARG, PLAT, |
| | | | PTGS2, CETP, AGTR1, HMGCR, |
| | | | IGF1, SELE, REN, PPARA, PON1, |
| | | | KNG1, CCL2, LPL, VWF, F2, |
| | | | ICAM1, TGFB, NPPA, IL10, EPO, |
| | | | SOD1, VCAM1, IFNG, LPA, MPO, |
| | | | ESR1, MAPK, HP, F3, CST3, COG2, |
| | | | MMP9, SERPINC1, F8, HMOX1, |
| | | | APOC3, IL8, PROL1, CBS, NOS2, |
| | | | TLR4, SELP, ABCA1, AGT, LDLR, |
| | | | GPT, VEGFA, NR3C2, IL18, NOS1, |
| | | | NR3C1, FGB, HGF, IL1A, AKT1, |
| | | | LIPC, HSPD1, MAPK14, SPP1, |
| | | | ITGB3, CAT, UTS2, THBD, F10, CP, |
| | | | TNFRSF11B, EGFR, MMP2, PLG, |
| | | | NPY, RHOD, MAPK8, MYC, FN1, |
| | | | CMA1, PLAU, GNB3, ADRB2, |
| | | | SOD2, F5, VDR, ALOX5, HLA- |
| | | | DRB1, PARP1, CD40LG, PON2, |
| | | | AGER, IRS1, PTGS1, ECE1, F7, |
| | | | IRMN, EPHX2, IGFBP1, MAPK10, |
| | | | FAS, ABCB1, JUN, IGFBP3, CD14, |
| | | | PDE5A, AGTR2, CD40, LCAT, |
| | | | CCR5, MMP1, TIMP1, ADM, |
| | | | DYT10, STAT3, MMP3, ELN, USF1, |
| | | | CFH, HSPA4, MMP12, MME, F2R, |
| | | | SELL, CTSB, ANXA5, ADRB1, |
| | | | CYBA, FGA, GGT1, LIPG, HIF1A, |
| | | | CXCR4, PROC, SCARB1, CD79A, |
| | | | PLTP, ADD1, FGG, SAA1, KCNH2, |
| | | | DPP4, NPR1, VTN, KIAA0101, FOS, |
| | | | TLR2, PPIG, IL1R1, AR, CYP1A1, |
| | | | SERPINA1, MTR, RBP4, APOA4, |
| | | | CDKN2A, FGF2, EDNRB, ITGA2, |
| | | | VLA-2, CABIN1, SHBG, HMGB1, |
| | | | HSP90B2P, CYP3A4, GJA1, CAV1, |
| | | | ESR2, LTA, GDF15, BDNF, |
| | | | CYP2D6, NGF, SP1, TGIF1, SRC, |
| | | | EGF, PIK3CG, HLA-A, KCNQ1, |
| | | | CNR1, FBN1, CHKA, BEST1, |
| | | | CTNNB1, IL2, CD36, PRKAB1, TPO, |
| | | | ALDH7A1, CX3CR1, TH, F9, CH1, |
| | | | TF, HFE, IL17A, PTEN, GSTM1, |
| | | | DMD, GATA4, F13A1, TTR, FABP4, |
| | | | PON3, APOC1, INSR, TNFRSF1B, |
| | | | HTR2A, CSF3, CYP2C9, TXN, |
| | | | CYP11B2, PTH, CSF2, KDR, |
| | | | PLA2G2A, THBS1, GCG, RHOA, |
| | | | ALDH2, TCF7L2, NFE2L2, |
| | | | NOTCH1, UGT1A1, IFNA1, PPARD, |
| | | | SIRT1, GNHR1, PAPPA, ARR3, |
| | | | NPPC, AHSP, PTK2, IL13, MTOR, |
| | | | ITGB2, GSTT1, IL6ST, CPB2, |
| | | | CYP1A2, HNF4A, SLC64A, |
| | | | PLA2G6, TNFSF11, SLC8A1, F2RL1, |
| | | | AKR1A1, ALDH9A1, BGLAP, |
| | | | MTTP, MTRR, SULT1A3, RAGE, |
| | | | C4B, P2RY12, RNLS, CREB1, |
| | | | POMC, RAC1, LMNA, CD59, |
| | | | SCM5A, CYP1B1, MIF, MMP13, |
| | | | TIMP2, CYP19A1, CUP21A2, |
| | | | PTPN22, MYH14, MBL2, SELPLG, |
| | | | AOC3, CTSL1, PCNA, IGF2, ITGB1, |
| | | | CAST, CXCL12, IGHE, KCNE1, |
| | | | TFRC, COL1A1, COL1A2, IL2RB, |
| | | | PLA2G10, ANGPT2, PROCR, NOX4, |
| | | | HAMP, PTPN11, SLCA1, IL2RA, |
| | | | CCL5, IRF1, CF:AR, CA:CA, EIF4E, |
| | | | GSTP1, JAK2, CYP3A5, HSPG2, |
| | | | CCL3, MYD88, VIP, SOAT1, |
| | | | ADRBK1, NR4A2, MMP8, NPR2, |
| | | | GCH1, EPRS, PPARGC1A, F12, |
| | | | PECAM1, CCL4, CERPINA34, |
| | | | CASR, FABP2, TTF2, PROS1, CTF1, |
| | | | SGCB, YME1L1, CAMP, ZC3H12A, |
| | | | AKR1B1, MMP7, AHR, CSF1, |
| | | | HDAC9, CTGF, KCNMA1, UGT1A, |
| | | | PRKCA, COMT, S100B, EGR1, PRL, |
| | | | IL15, DRD4, CAMK2G, SLC22A2, |
| | | | CCL11, PGF, THPO, GP6, TACR1, |
| | | | NTS, HNF1A, SST, KCDN1, |
| | | | LOC646627, TBXAS1, CUP2J2, |
| | | | TBXA2R, ADH1C, ALOX12, AHSG, |
| | | | BHMT, GJA4, SLC25A4, ACLY, |
| | | | ALOX5AP, NUMA1, CYP27B1, |
| | | | CYSLTR2, SOD3, LTC4S, UCN, |
| | | | GHRL, APOC2, CLEC4A, |
| | | | KBTBD10, TNC, TYMS, SHC1, |
| | | | LRP1, SOCS3, ADH1B, KLK3, |
| | | | HSD11B1, VKORC1, SERPINB2, |
| | | | TNS1, RNF19A, EPOR, ITGAM, |
| | | | PITX2, MAPK7, FCGR3A, LEEPR, |
| | | | ENG, GPX1, GOT2, HRH1, NR112, |
| | | | CRH, HTR1A, VDAC1, HPSE, |
| | | | SFTPD, TAP2, RMF123, PTK2Bm |
| | | | NTRK2, IL6R, ACHE, GLP1R, GHR, |
| | | | GSR, NQO1, NR5A1, GJB2, |
| | | | SLC9A1, MAOA, PCSK9, FCGR2A, |
| | | | SERPINF1, EDN3, UCP2, TFAP2A, |
| | | | C4BPA, SERPINF2, TYMP, ALPP, |
| | | | CXCR2, SLC3A3, ABCG2, ADA, |
| | | | JAK3, HSPA1A, FASN, FGF1, F11, |
| | | | ATP7A, CR1, GFPA, ROCK1, |
| | | | MECP2, MYLK, BCHE, LIPE, |
| | | | ADORA1, WRN, CXCR3, CD81, |
| | | | SMAD7, LAMC2, MAP3K5, CHGA, |
| | | | IAPP, RHO, ENPP1, PTHLH, NRG1, |
| | | | VEGFC, ENPEP, CEBPB, NAGLU,. |
| | | | F2RL3, CX3CL1, BDKRB1, |
| | | | ADAMTS13, ELANE, ENPP2, CISH, |
| | | | GAST, MYOC, ATP1A2, NF1, GJB1, |
| | | | MEF2A, VCL, BMPR2, TUBB, |
| | | | CDC42, KRT18, HSF1, MYB, |
| | | | PRKAA2, ROCK2, TFP1, PRKG1, |
| | | | BMP2, CTNND1, CTH, CTSS, |
| | | | VAV2, NPY2R, IGFBP2, CD28, |
| | | | GSTA1, PPIA, APOH, S100A8, IL11, |
| | | | ALOX15, FBLN1, NR1H3, SCD, GIP, |
| | | | CHGB, PRKCB, SRD5A1,HSD11B2, |
| | | | CALCRL, GALNT2, ANGPTL4, |
| | | | KCNN4, PIK3C2A, HBEGF, |
| | | | CYP7A1, HLA-DRB5, BNIP3, |
| | | | GCKR, S100A12, PADI4, HSPA14, |
| | | | CXCR1, H19, KRTAP19-3, IDDM2, |
| | | | RAC2, YRY1, CLOCK, NGFR, DBH, |
| | | | CHRNA4, CACNA1C, PRKAG2, |
| | | | CHAT, PTGDS, NR1H2, TEK, |
| | | | VEGFB, MEF2C, MAPKAPK2, |
| | | | TNFRSF11A, HSPA9, CYSLTR1, |
| | | | MAT1A, OPRL1, IMPA1, CLCN2, |
| | | | DLD, PSMA6, PSMB8, CHI3L1, |
| | | | ALDH1B1, PARP2, STAR, LBP, |
| | | | ABCC6, RGS2, EFNB2, GJB6, |
| | | | APOA2, AMPD1, DYSF, |
| | | | FDFT1, EMD2, CCR6, GJB3, IL1RL1, |
| | | | ENTPD1, BBS4, CELSR2, F11R, |
| | | | RAPGEF3, HYAL1, ZNF259, |
| | | | ATOX1, ATF6, KHK, SAT1, GGH, |
| | | | TIMP4, SLC4A4, PDE2A, PDE3B, |
| | | | FADS1, FADS2, TMSB4X, TXNIP, |
| | | | LIMS1, RHOB, LY96, FOXO1, |
| | | | PNPLA2,TRH, GJC1, S:C17A5, FTO, |
| | | | GJD2, PRSC1, CASP12, GPBAR1, |
| | | | PXK, IL33, TRIB1, PBX4, NUPR1, |
| | | | 15-SEP, CILP2, TERC, GGT2, |
| | | | MTCO1, UOX, AVP |
| Cataract | eye | | CRYAA, CRYA1, CRYBB2, CRYB2, |
| | | | PITX3, BFSP2, CP49, CP47, CRYAA, |
| | | | CRYA1, PAX6, AN2, MGDA, |
| | | | CRYBA1, CRYB1, CRYGC, CRYG3, |
| | | | CCL, LIM2, MP19, CRYGD, CRYG4, |
| | | | BFSP2, CP49, CP47, HSF4, CTM, |
| | | | HSF4, CTM, MIP, AQP0, CRYAB, |
| | | | CRYA2, CTPP2, CRYBB1, CRYGD, |
| | | | CRYG4, CRYBB2, CRYB2, CRYGC, |
| | | | CRYG3, CCL, CRYAA, CRYA1, |
| | | | GJA8, CX50, CAE1, GJA3, CX46, |
| | | | CZP3, CAE3, CCM1, CAM, KRIT1 |
| CDKL-5 Deficiencies or | Brain, CNS | | CDKL5 |
| Mediated Diseases | | | |
| Charcot-Marie-Tooth (CMT) | Nervous system | Muscles | PMP22 (CMT1A and E), MPZ |
| disease (Types 1, 2, 3, 4,) | | (dystrophy) | (CMT1B), LITAF (CMT1C), EGR2 |
| | | | (CMT1D), NEFL (CMT1F), GJB1 |
| | | | (CMT1X), MFN2 (CMT2A), KIF1B |
| | | | (CMT2A2B), RAB7A (CMT2B), |
| | | | TRPV4 (CMT2C), GARS (CMT2D), |
| | | | NEFL (CMT2E), GAPD1 (CMT2K), |
| | | | HSPB8 (CMT2L), DYNC1H1, |
| | | | CMT20), LRSAM1 (CMT2P), |
| | | | IGHMBP2 (CMT2S), MORC2 |
| | | | (CMT2Z), GDAP1 (CMT4A), |
| | | | MTMR2 or SBF2/MTMR13 |
| | | | (CMT4B), SH3TC2 (CMT4C), |
| | | | NDRG1 (CMT4D), PRX (CMT4F), |
| | | | FIG4 (CMT4J), NT-3 |
| Chédiak-Higashi Syndrome | Immune system | Skin, hair, eyes, | LYST |
| | | neurons | |
| Choroidermia | | | CHM, REP1, |
| Chorioretinal atrophy | eye | | PRDM13, RGR, TEAD1 |
| Chronic Granulomatous Disease | Immune system | | CYBA, CYBB, NCF1, NCF2, NCF4 |
| Chronic Mucocutaneous | Immune system | | AIRE, CARD9, CLEC7A IL12B, |
| Candidiasis | | | IL12B1, IL1F, IL17RA, IL17RC, |
| | | | RORC, STAT1, STAT3, TRAF31P2 |
| Cirrhosis | liver | | KRT18, KRT8, CIRH1A, NAIC, |
| | | | TEX292, KIAA1988 |
| Colon cancer (Familial | Gastrointestinal | | FAP: APC HNPCC: |
| adenomatous polyposis (FAP) | | | MSH2, MLH1, PMS2, SH6, PMS1 |
| and hereditary nonpolyposis | | | |
| colon cancer (HNPCC)) | | | |
| Combined Immunodeficiency | Immune System | | IL2RG, SCIDX1, SCIDX, IMD4); |
| | | | HIV-1 (CCL5, SCYA5, D17S136E, |
| | | | TCP228 |
| Cone(-rod) dystrophy | eye | | AIPL1, CRX, GUA1A, GUCY2D, |
| | | | PITPM3, PROM1, PRPH2, RIMS1, |
| | | | SEMA4A, ABCA4, ADAM9, ATF6, |
| | | | C21ORF2, C8ORF37, CACNA2D4, |
| | | | CDHR1, CERKL, CNGA3, CNGB3, |
| | | | CNNM4, CNAT2, IFT81, KCNV2, |
| | | | PDE6C, PDE6H, POC1B, RAX2, |
| | | | RDH5, RPGRIP1, TTLL5, RetCG1, |
| | | | GUCY2E |
| Congenital Stationary Night | eye | | CABP4, CACNA1F, CACNA2D4, |
| Blindness | | | GNAT1, CPR179, GRK1, GRM6, |
| | | | LRIT3, NYX, PDE6B, RDH5, RHO, |
| | | | RLBP1, RPE65, SAG, SLC24A1, |
| | | | TRPM1, |
| Congenital Fructose Intolerance | Metabolism | | ALDOB |
| Cori's Disease (Glycogen Storage | Various- | | AGL |
| Disease Type III) | wherever | | |
| | glycogen | | |
| | accumulates, | | |
| | particularly | | |
| | liver, heart, | | |
| | skeletal muscle | | |
| Corneal clouding and dystrophy | eye | | APOA1, TGFBI, CSD2, CDGG1, |
| | | | CSD, BIGH3, CDG2, TACSTD2, |
| | | | TROP2, M1S1, VSX1, RINX, PPCD, |
| | | | PPD, KTCN, COL8A2, FECD, |
| | | | PPCD2, PIP5K3, CFD |
| Cornea plana congenital | | | KERA, CNA2 |
| Cri du chat Syndrome, also | | | Deletions involving only band 5p15.2 |
| known as 5p syndrome and cat | | | to the entire short arm of chromosome |
| cry syndrome | | | 5, e.g. CTNND2, TERT, |
| Cystic Fibrosis (CF) | Lungs and | Pancreas, liver, | CTFR, ABCC7, CF, MRP7, SCNN1A, |
| | respiratory | digestive | those described in WO2015157070 |
| | system | system, | |
| | | reproductive | |
| | | system, | |
| | | exocrine, glands, | |
| Diabetic nephropathy | kidney | | Gremlin, 12/15- lipoxygenase, TIM44, |
| Dent Disease (Types 1 and 2) | Kidney | | Type 1: CLCN5, Type 2: ORCL |
| Dentatorubro-Pallidoluysian | CNS, brain, | | Atrophin-1 and Atn1 |
| Atrophy (DRPLA) (aka Haw | muscle | | |
| River and Naito-Oyanagi | | | |
| Disease) | | | |
| Down Syndrome | various | | Chromosome 21 trisomy |
| Drug Addiction | Brain | | Prkce; Drd2; Drd4; ABAT; |
| | | | GRIA2; Grm5; Grin1; Htr1b; Grin2a; |
| | | | Drd3; Pdyn; Gria1 |
| Duane syndrome (Types 1, 2, and | eye | | CHN1, indels on chromosomes 4 and 8 |
| 3, including subgroups A, B and | | | |
| C). Other names for this | | | |
| condition include: Duane's | | | |
| Retraction Syndrome (or DR | | | |
| syndrome), Eye Retraction | | | |
| Syndrome, Retraction Syndrome, | | | |
| Congenital retraction syndrome | | | |
| and Stilling-Turk-Duane | | | |
| Syndrome | | | |
| Duchenne muscular dystrophy | muscle | Cardiovascular, | DMD, BMD, dystrophin gene, intron |
| (DMD) | | respiratory | flanking exon 51 of DMD gene, exon |
| | | | 51 mutations in DMD gene, see also |
| | | | WO2013163628 and US Pat. Pub. |
| | | | 20130145487 |
| Edward's Syndrome | | | Complete or partial trisomy of |
| (Trisomy 18) | | | chromosome 18 |
| Ehlers-Danlos Syndrome (Types | Various | | COL5A1, COL5A2, COL1A1, |
| I-VI) | depending on | | COL3A1, TNXB, PLOD1, COL1A2, |
| | type: including | | FKBP14 and ADAMTS2 |
| | musculoskeletal, | | |
| | eye, vasculature, | | |
| | immune, and | | |
| | skin | | |
| Emery-Dreifuss muscular | muscle | | LMNA, LMN1, EMD2, FPLD, |
| dystrophy | | | CMD1A, HGPS, LGMD1B, LMNA, |
| | | | LMN1, EMD2, FPLD, CMD1A |
| Enhanced S-Cone Syndrome | eye | | NR2E3, NRL |
| Fabry's Disease | Various - | | GLA |
| | including skin, | | |
| | eyes, and | | |
| | gastrointestinal | | |
| | system, kidney, | | |
| | heart, brain, | | |
| | nervous system | | |
| Facioscapulohumeral muscular | muscles | | FSHMD1A, FSHD1A, FRG1, |
| dystrophy | | | |
| Factor H and Factor H-like 1 | blood | | HF1, CFH, HUS |
| Factor V Leiden thrombophilia | blood | | Factor V (F5) |
| and Factor V deficiency | | | |
| Factor V and Factor VII | blood | | MCFD2 |
| deficiency | | | |
| Factor VII deficiency | blood | | F7 |
| Factor X deficiency | blood | | F10 |
| Factor XI deficiency | blood | | F11 |
| Factor XII deficiency | blood | | F12, HAF |
| Factor XIIIA deficiency | blood | | F13A1, F13A |
| Factor XIIIB deficiency | blood | | F13B |
| Familial Hypercholestereolemia | Cardiovascular | | APOB, LDLR, PCSK9 |
| | system | | |
| Familial Mediterranean Fever | Various- | Heart, kidney, | MEFV |
| (FMF) also called recurrent | organs/tissues | brain/CNS, | |
| polyserositis or familial | with serous or | reproductive | |
| paroxysmal polyserositis | synovial | organs | |
| | membranes, | | |
| | skin, joints | | |
| Fanconi Anemia | Various - blood | | FANCA, FACA, FA1, FA, FAA, |
| | (anemia), | | FAAP95, FAAP90, FLJ34064, |
| | immune system, | | FANCC, FANCG, RAD51, BRCA1, |
| | cognitive, | | BRCA2, BRIP1, BACH1, FANCJ, |
| | kidneys, eyes, | | FANCB, FANCD1, FANCD2, |
| | musculoskeletal | | FANCD, FAD, FANCE, FACE, |
| | | | FANCF, FANCI, ERCC4, FANCL, |
| | | | FANCM, PALB2, RAD51C, SLX4, |
| | | | UBE2T, FANCB, XRCC9, PHF9, |
| | | | KIAA1596 |
| Fanconi Syndrome Types I | kidneys | | FRTS1, GATM |
| (Childhood onset) and II (Adult | | | |
| Onset) | | | |
| Fragile X syndrome and related | brain | | FMR1, FMR2; FXR1; FXR2; |
| disorders | | | mGLUR5 |
| Fragile XE Mental Retardation | Brain, nervous | | FMR1 |
| (aka Martin Bell syndrome) | system | | |
| Friedreich Ataxia (FRDA) | Brain, nervous | heart | FXN/X25 |
| | system | | |
| Fuchs endothelial corneal | Eye | | TCF4; COL8A2 |
| dystrophy | | | |
| Galactosemia | Carbohydrate | Various-where | GALT, GALK1, and GALE |
| | metabolism | galactose | |
| | disorder | accumulates - | |
| | | liver, brain, eyes | |
| Gastrointestinal Epithelial | | | CISH |
| Cancer, GI cancer | | | |
| Gaucher Disease (Types 1, 2, and | Fat metabolism | Various-liver, | GBA |
| 3, as well as other unusual forms | disorder | spleen, blood, | |
| that may not fit into these types) | | CNS, skeletal | |
| | | system | |
| Griscelli syndrome | | | |
| Glaucoma | eye | | MYOC, TIGR, GLC1A, JOAG, |
| | | | GPOA, OPTN, GLC1E, FIP2, HYPL, |
| | | | NRP, CYP1B1, GLC3A, OPA1, NTG, |
| | | | NPG, CYP1B1, GLC3A, those |
| | | | described in WO2015153780 |
| Glomerulo sclerosis | kidney | | CC chemokine ligand 2 |
| Glycogen Storage Diseases | Metabolism | | SLC2A2, GLUT2, G6PC, G6PT, |
| Types I-VI -See also Cori's | Diseases | | G6PT1, GAA, LAMP2, LAMPB, |
| Disease, Pompe's Disease, | | | AGL, GDE, GBE1, GYS2, PYGL, |
| McArdle's disease, Hers Disease, | | | PFKM, see also Cori's Disease, |
| and Von Gierke's disease | | | Pompe's Disease, McArdle's disease, |
| | | | Hers Disease, and Von Gierke's |
| | | | disease |
| RBC Glycolytic enzyme | blood | | any mutations in a gene for an enzyme |
| deficiency | | | in the glycolysis pathway including |
| | | | mutations in genes for hexokinases I |
| | | | and II, glucokinase, phosphoglucose |
| | | | isomerase, phosphofructokinase, |
| | | | aldolase Bm triosephosphate |
| | | | isomerease, glyceraldehydee-3- |
| | | | phosphate dehydrogenase, |
| | | | phosphoglycerokinase, |
| | | | phosphoglycerate mutase, enolase I, |
| | | | pyruvate kinase |
| Hartnup's disease | Malabsorption | Various- brain, | SLC6A19 |
| | disease | gastrointestinal, | |
| | | skin, | |
| Hearing Loss | ear | | NOX3, Hes5, BDNF, |
| Hemochromatosis (HH) | Iron absorption | Various- | HFE and H63D |
| | regulation | wherever iron | |
| | disease | accumulates, | |
| | | liver, heart, | |
| | | pancreas, joints, | |
| | | pituitary gland | |
| Hemophagocytic | blood | | PRF1, HPLH2, UNC13D, MUNC13- |
| lymphohistiocytosis disorders | | | 4, HPLH3, HLH3, FHL3 |
| Hemorrhagic disorders | blood | | PI, ATT, F5 |
| Hers disease (Glycogen storage | liver | muscle | PYGL |
| disease Type VI) | | | |
| Hereditary angioedema (HAE) | | | kalikrein B1 |
| Hereditary Hemorrhagic | Skin and | | ACVRL1, ENG and SMAD4 |
| Telangiectasia (Osler-Weber- | mucous | | |
| Rendu Syndrome) | membranes | | |
| Hereditary Spherocytosis | blood | | NK1, EPB42, SLC4A1, SPTA1, and |
| | | | SPTB |
| Hereditary Persistence of Fetal | blood | | HBG1, HBG2, BCL11A, promoter |
| Hemoglobin | | | region of HBG 1 and/or 2 (in the |
| | | | CCAAT box) |
| Hemophilia (hemophilia A | blood | | A: FVIII, F8C, HEMA |
| (Classic) a B (aka Christmas | | | B: FVIX, HEMB |
| disease) and C) | | | C: F9, F11 |
| Hepatic adenoma | liver | | TCF1, HNF1A, MODY3 |
| Hepatic failure, early onset, and | liver | | SCOD1, SCO1 |
| neurologic disorder | | | |
| Hepatic lipase deficiency | liver | | LIPC |
| Hepatoblastoma, cancer and | liver | | CTNNB1, PDGFRL, PDGRL, PRLTS, |
| carcinomas | | | AXIN1, AXIN, CTNNB1, TP53, P53, |
| | | | LFS1, IGF2R, MPRI, MET, CASP8, |
| | | | MCH5 |
| Hermansky-Pudlak syndrome | Skin, eyes, | | HPS1, HPS3, HPS4, HPS5, HPS6, |
| | blood, lung, | | HPS7, DTNBP1, BLOC1, BLOC1S2, |
| | kidneys, | | BLOC3 |
| | intestine | | |
| HIV susceptibility or infection | Immune system | | IL10, CSIF, CMKBR2, CCR2, |
| | | | CMKBR5, CCCKR5 (CCR5), those in |
| | | | WO2015148670A1 |
| Holoprosencephaly (HPE) | brain | | ACVRL1, ENG, SMAD4 |
| (Alobar, Semilobar, and Lobar) | | | |
| Homocystinuria | Metabolic | Various- | CBS, MTHFR, MTR, MTRR, and |
| | disease | connective | MMADHC |
| | | tissue, muscles, | |
| | | CNS, | |
| | | cardiovascular | |
| | | system | |
| HPV | | | HPV16 and HPV18 E6/E7 |
| HSV1, HSV2, and related | eye | | HSV1 genes (immediate early and late |
| keratitis | | | HSV-1 genes (UL1, 1.5, 5, 6, 8, 9, 12, |
| | | | 15, 16, 18, 19, 22, 23, 26, 26.5, 27, 28, |
| | | | 29, 30, 31, 32, 33, 34, 35, 36, 37, 38, |
| | | | 42, 48, 49.5, 50, 52, 54, S6, RL2, RS1, |
| | | | those described in WO2015153789, |
| | | | WO2015153791 |
| Hunter's Syndrome (aka | Lysosomal | Various- liver, | IDS |
| Mucopolysaccharidosis type II) | storage disease | spleen, eye, | |
| | | joint, heart, | |
| | | brain, skeletal | |
| Huntington's disease (HD) and | Brain, nervous | | HD, HTT, IT15, PRNP, PRIP, JPH3, |
| HD-like disorders | system | | JP3, HDL2, TBP, SCA17, PRKCE; |
| | | | IGF1; EP300; RCOR1; PRKCZ; |
| | | | HDAC4; and TGM2, and those |
| | | | described in WO2013130824, |
| | | | WO2015089354 |
| Hurler's Syndrome (aka | Lysosomal | Various- liver, | IDUA, α-L-iduronidase |
| mucopolysaccharidosis type I H, | storage disease | spleen, eye, | |
| MPS IH) | | joint, heart, | |
| | | brain, skeletal | |
| Hurler-Scheie syndrome (aka | Lysosomal | Various- liver, | IDUA, α-L-iduronidase |
| mucopolysaccharidosis type I H- | storage disease | spleen, eye, | |
| S, MPS I H-S) | | joint, heart, | |
| | | brain, skeletal | |
| hyaluronidase deficiency (aka | Soft and | | HYAL1 |
| MPS IX) | connective | | |
| | tissues | | |
| Hyper IgM syndrome | Immune system | | CD40L |
| Hyper- tension caused renal | kidney | | Mineral corticoid receptor |
| damage | | | |
| Immunodeficiencies | Immune System | | CD3E, CD3G, AICDA, AID, HIGM2, |
| | | | TNFRSF5, CD40, UNG, DGU, |
| | | | HIGM4, TNFSF5, CD40LG, HIGM1, |
| | | | IGM, FOXP3, IPEX, AIID, XPID, |
| | | | PIDX, TNFRSF14B, TACI |
| Inborn errors of metabolism: | Metabolism | Various organs | See also: Carbohydrate metabolism |
| including urea cycle disorders, | diseases, liver | and cells | disorders (e.g. galactosemia), Amino |
| organic acidemias), fatty acid | | | acid Metabolism disorders (e.g. |
| oxidation defects, amino | | | phenylketonuria), Fatty acid |
| acidopathies, carbohydrate | | | metabolism (e.g. MCAD deficiency), |
| disorders, mitochondrial | | | Urea Cycle disorders (e.g. |
| disorders | | | Citrullinemia), Organic acidemias (e.g. |
| | | | Maple Syrup Urine disease), |
| | | | Mitochondrial disorders (e.g. |
| | | | MELAS), peroxisomal disorders (e.g. |
| | | | Zellweger syndrome) |
| Inflammation | Various | | IL-10; IL-1 (IL-1a; IL-1b); IL-13; IL- |
| | | | 17 (IL-17a (CTLA8); IL- |
| | | | 17b; IL-17c; IL-17d; IL-17f); II-23; |
| | | | Cx3cr1; ptpn22; TNFa; |
| | | | NOD2/CARD15 for IBD; IL-6; IL-12 |
| | | | (IL-12a; IL-12b); |
| | | | CTLA4; Cx3cl1 |
| Inflammatory Bowel Diseases | Gastrointestinal | Joints, skin | NOD2, IRGM, LRRK2, ATG5, |
| (e.g. Ulcerative Colitis and | | | ATG16L1, IRGM, GATM, ECM1, |
| Chron's Disease) | | | CDH1, LAMB1, HNF4A, GNA12, |
| | | | IL10, CARD9/15. CCR6, IL2RA, |
| | | | MST1, TNFSF15, REL, STAT3, |
| | | | IL23R, IL12B, FUT2 |
| Interstitial renal fibrosis | kidney | | TGF-β type II receptor |
| Job's Syndrome (aka Hyper IgE | Immune System | | STAT3, DOCK8 |
| Syndrome) | | | |
| Juvenile Retinoschisis | eye | | RS1, XLRS1 |
| Kabuki Syndrome 1 | | | MLL4, KMT2D |
| Kennedy Disease (aka | Muscles, brain, | | SBMA/SMAX1/AR |
| Spinobulbar Muscular Atrophy) | nervous system | | |
| Klinefelter syndrome | Various- | | Extra X chromosome in males |
| | particularly | | |
| | those involved | | |
| | in development | | |
| | of male | | |
| | characteristics | | |
| Lafora Disease | Brain, CNS | | EMP2A and EMP2B |
| Leber Congenital Amaurosis | eye | | CRB1, RP12, CORD2, CRD, CRX, |
| | | | IMPDH1, OTX2, AIPL1, CABP4, |
| | | | CCT2, CEP290, CLUAP1, CRB1, |
| | | | CRX, DTHD1, GDF6, GUCY2D, |
| | | | IFT140, IQCB1, KCNJ13, LCA5, |
| | | | LRAT, NMNAT1, PRPH2, RD3, |
| | | | RDH12, RPE65, RP20, RPGRIP1, |
| | | | SPATA7, TULP1, LCA1, LCA4, |
| | | | GUC2D, CORD6, LCA3, |
| Lesch-Nyhan Syndrome | Metabolism | Various - joints, | HPRT1 |
| | disease | cognitive, brain, | |
| | | nervous system | |
| Leukocyte deficiencies and | blood | | ITGB2, CD18, LCAMB, LAD, |
| disorders | | | EIF2B1, EIF2BA, EIF2B2, EIF2B3, |
| | | | EIF2B5, LVWM, CACH, CLE, |
| | | | EIF2B4 |
| Leukemia | Blood | | TAL1, TCL5, SCL, TAL2, FLT3, |
| | | | NBS1, NBS, ZNFN1A1, IK1, LYF1, |
| | | | HOXD4, HOX4B, BCR, CML, PHL, |
| | | | ALL, ARNT, KRAS2, RASK2, |
| | | | GMPS, AF10, ARHGEF12, LARG, |
| | | | KIAA0382, CALM, CLTH, CEBPA, |
| | | | CEBP, CHIC2, BTL, FLT3, KIT, |
| | | | PBT, LPP, NPM1, NUP214, D9S46E, |
| | | | CAN, CAIN, RUNX1, CBFA2, |
| | | | AML1, WHSC1L1, NSD3, FLT3, |
| | | | AF1Q, NPM1, NUMA1, ZNF145, |
| | | | PLZF, PML, MYL, STAT5B, AF10, |
| | | | CALM, CLTH, ARL11, ARLTS1, |
| | | | P2RX7, P2X7, BCR, CML, PHL, |
| | | | ALL, GRAF, NF1, VRNF, WSS, |
| | | | NFNS, PTPN11, PTP2C, SHP2, NS1, |
| | | | BCL2, CCND1, PRAD1, BCL1, |
| | | | TCRA, GATA1, GF1, ERYF1, NFE1, |
| | | | ABL1, NQO1, DIA4, NMOR1, |
| | | | NUP214, D9S46E, CAN, CAIN |
| Limb-girdle muscular dystrophy | muscle | | LGMD |
| diseases | | | |
| Lowe syndrome | brain, eyes, | | OCRL |
| | kidneys | | |
| Lupus glomerulo- nephritis | kidney | | MAPK1 |
| Machado- | Brain, CNS, | | ATX3 |
| Joseph's Disease (also known as | muscle | | |
| Spinocerebellar ataxia Type 3) | | | |
| Macular degeneration | eye | | ABC4, CBC1, CHM1, APOE, |
| | | | C1QTNF5, C2, C3, CCL2, CCR2, |
| | | | CD36, CFB, CFH, CFHR1, CFHR3, |
| | | | CNGB3, CP, CRP, CST3, CTSD, |
| | | | CX3CR1, ELOVL4, ERCC6, FBLN5, |
| | | | FBLN6, FSCN2, HMCN1, HIRA1, |
| | | | IL6, IL8, PLEKHA1, PROM1, |
| | | | PRPH2, RPGR, SERPING1, TCOF1, |
| | | | TIMP3, TLR3 |
| Macular Dystrophy | eye | | BEST1, C1QTNF5, CTNNA1, |
| | | | EFEMP1, ELOVL4, FSCN2, |
| | | | GUCA1B, HMCN1, IMPG1, OTX2, |
| | | | PRDM13, PROM1, PRPH2, RP1L1, |
| | | | TIMP3, ABCA4, CFH, DRAM2, |
| | | | IMG1, MFSD8, ADMD, STGD2, |
| | | | STGD3, RDS, RP7, PRPH, AVMD, |
| | | | AOFMD, VMD2 |
| Malattia Leventinesse | eye | | EFEMP1, FBLN3 |
| Maple Syrup Urine Disease | Metabolism | | BCKDHA, BCKDHB, and DBT |
| | disease | | |
| Marfan syndrome | Connective | Musculoskeletal | FBN1 |
| | tissue | | |
| Maroteaux-Lamy Syndrome (aka | Musculoskeletal | Liver, spleen | ARSB |
| MPS VI) | system, nervous | | |
| | system | | |
| McArdle's Disease (Glycogen | Glycogen | muscle | PYGM |
| Storage Disease Type V) | storage disease | | |
| Medullary cystic kidney disease | kidney | | UMOD, HNFJ, FJHN, MCKD2, |
| | | | ADMCKD2 |
| Metachromatic leukodystrophy | Lysosomal | Nervous system | ARSA |
| | storage disease | | |
| Methylmalonic acidemia (MMA) | Metabolism | | MMAA, MMAB, MUT, MMACHC, |
| | disease | | MMADHC, LMBRD1 |
| Morquio Syndrome (aka MPS IV | Connective | heart | GALNS |
| A and B) | tissue, skin, | | |
| | bone, eyes | | |
| Mucopolysaccharidosis diseases | Lysosomal | | See also Hurler/Scheie syndrome, |
| (Types I H/S, I H, II, III A B and | storage disease - | | Hurler disease, Sanfillipo syndrome, |
| C, I S, IVA and B, IX, VII, and | affects various | | Scheie syndrome, Morquio syndrome, |
| VI) | organs/tissues | | hyaluronidase deficiency, Sly |
| | | | syndrome, and Maroteaux-Lamy |
| | | | syndrome |
| Muscular Atrophy | muscle | | VAPB, VAPC, ALS8, SMN1, SMA1, |
| | | | SMA2, SMA3, SMA4, BSCL2, |
| | | | SPG17, GARS, SMAD1, CMT2D, |
| | | | HEXB, IGHMBP2, SMUBP2, |
| | | | CATF1, SMARD1 |
| Muscular dystrophy | muscle | | FKRP, MDC1C, LGMD2I, LAMA2, |
| | | | LAMM, LARGE, KIAA0609, |
| | | | MDC1D, FCMD, TTID, MYOT, |
| | | | CAPN3, CANP3, DYSF, LGMD2B, |
| | | | SGCG, LGMD2C, DMDA1, SCG3, |
| | | | SGCA, ADL, DAG2, LGMD2D, |
| | | | DMDA2, SGCB, LGMD2E, SGCD, |
| | | | SGD, LGMD2F, CMD1L, TCAP, |
| | | | LGMD2G, CMD1N, TRIM32, HT2A, |
| | | | LGMD2H, FKRP, MDC1C, LGMD2I, |
| | | | TTN, CMD1G, TMD, LGMD2J, |
| | | | POMT1, CAV3, LGMD1C, SEPN1, |
| | | | SELN, RSMD1, PLEC1, PLTN, EBS1 |
| Myotonic dystrophy (Type 1 and | Muscles | Eyes, heart, | CNBP (Type 2) and DMPK (Type 1) |
| Type 2) | | endocrine | |
| Neoplasia | | | PTEN; ATM; ATR; EGFR; ERBB2; |
| | | | ERBB3; ERBB4; |
| | | | Notch1; Notch2; Notch3; Notch4; |
| | | | AKT; AKT2; AKT3; HIF; |
| | | | HIF1a; HIF3a; Met; HRG; Bcl2; |
| | | | PPAR alpha; PPAR |
| | | | gamma; WT1 (Wilms Tumor); FGF |
| | | | Receptor Family |
| | | | members (5 members: 1, 2, 3, 4, 5); |
| | | | CDKN2a; APC; RB |
| | | | (retinoblastoma); MEN1; VHL; |
| | | | BRCA1; BRCA2; AR |
| | | | (Androgen Receptor); TSG101; IGF; |
| | | | IGF Receptor; Igf1 (4 |
| | | | variants); Igf2 (3 variants); Igf 1 |
| | | | Receptor; Igf 2 Receptor; |
| | | | Bax; Bcl2; caspases family (9 |
| | | | members: |
| | | | 1, 2, 3, 4, 6, 7, 8, 9, 12); Kras; Apc |
| Neurofibromatosis (NF) (NF1, | brain, spinal | | NF1, NF2 |
| formerly Recklinghausen's NF, | cord, nerves, | | |
| and NF2) | and skin | | |
| Niemann-Pick Lipidosis (Types | Lysosomal | Various- where | Types A and B: SMPD1; Type C: |
| A, B, and C) | Storage Disease | sphingomyelin | NPC1 or NPC2 |
| | | accumulates, | |
| | | particularly | |
| | | spleen, liver, | |
| | | blood, CNS | |
| Noonan Syndrome | Various - | | PTPN11, SOS1, RAF1 and KRAS |
| | musculoskeletal, | | |
| | heart, eyes, | | |
| | reproductive | | |
| | organs, blood | | |
| Norrie Disease or X-linked | eye | | NDP |
| Familial Exudative | | | |
| Vitreoretinopathy | | | |
| North Carolina Macular | eye | | MCDR1 |
| Dystrophy | | | |
| Osteogenesis imperfecta (OI) | bones, | | COL1A1, COL1A2, CRTAP, P3H |
| (Types I, II, III, IV, V, VI, VII) | musculoskeletal | | |
| Osteopetrosis | bones | | LRP5, BMND1, LRP7, LR3, OPPG, |
| | | | VBCH2, CLCN7, CLC7, OPTA2, |
| | | | OSTM1, GL, TCIRG1, TIRC7, |
| | | | OC116, OPTB1 |
| Patau's Syndrome | Brain, heart, | | Additional copy of chromosome 13 |
| (Trisomy 13) | skeletal system | | |
| Parkinson's disease (PD) | Brain, nervous | | SNCA (PARK1), UCHL1 (PARK 5), |
| | system | | and LRRK2 (PARK8), (PARK3), |
| | | | PARK2, PARK4, PARK7 (PARK7), |
| | | | PINK1 (PARK6); x-Synuclein, DJ-1, |
| | | | Parkin, NR4A2, NURR1, NOT, |
| | | | TINUR, SNCAIP, TBP, SCA17, |
| | | | NCAP, PRKN, PDJ, DBH, NDUFV2 |
| Pattern Dystrophy of the RPE | eye | | RDS/peripherin |
| Phenylketonuria (PKU) | Metabolism | Various due to | PAH, PKU1, QDPR, DHPR, PTS |
| | disorder | build-up of | |
| | | phenylalanine, | |
| | | phenyl ketones | |
| | | in tissues and | |
| | | CNS | |
| Polycystic kidney and hepatic | Kidney, liver | | FCYT, PKHD1, ARPKD, PKD1, |
| disease | | | PKD2, PKD4, PKDTS, PRKCSH, |
| | | | G19P1, PCLD, SEC63 |
| Pompe's Disease | Glycogen | Various - heart, | GAA |
| | storage disease | liver, spleen | |
| Porphyria (actually refers to a | Various- | | ALAD, ALAS2, CPOX, FECH, |
| group of different diseases all | wherever heme | | HMBS, PPOX, UROD, or UROS |
| having a specific heme | precursors | | |
| production process abnormality) | accumulate | | |
| posterior polymorphous corneal | eyes | | TCF4; COL8A2 |
| dystrophy | | | |
| Primary Hyperoxaluria (e.g. type | Various - eyes, | | LDHA (lactate dehydrogenase A) and |
| 1) | heart, kidneys, | | hydroxyacid oxidase 1 (HAO1) |
| | skeletal system | | |
| Primary Open Angle Glaucoma | eyes | | MYOC |
| (POAG) | | | |
| Primary sclerosing cholangitis | Liver, | | TCF4; COL8A2 |
| | gallbladder | | |
| Progeria (also called Hutchinson- | All | | LMNA |
| Gilford progeria syndrome) | | | |
| Prader-Willi Syndrome | Musculoskeletal | | Deletion of region of short arm of |
| | system, brain, | | chromosome 15, including UBE3A |
| | reproductive | | |
| | and endocrine | | |
| | system | | |
| Prostate Cancer | prostate | | HOXB13, MSMB, GPRC6A, TP53 |
| Pyruvate Dehydrogenase | Brain, nervous | | PDHA1 |
| Deficiency | system | | |
| Kidney/Renal carcinoma | kidney | | RLIP76, VEGF |
| Rett Syndrome | Brain | | MECP2, RTT, PPMX, MRX16, |
| | | | MRX79, CDKL5, STK9, MECP2, |
| | | | RTT, PPMX, MRX16, MRX79, x- |
| | | | Synuclein, DJ-1 |
| Retinitis pigmentosa (RP) | eye | | ADIPOR1, ABCA4, AGBL5, |
| | | | ARHGEF18, ARL2BP, ARL3, ARL6, |
| | | | BEST1, BBS1, BBS2, C2ORF71, |
| | | | C8ORF37, CA4, CERKL, CLRN1, |
| | | | CNGA1, CMGB1, CRB1, CRX, |
| | | | CYP4V2, DHDDS, DHX38, EMC1, |
| | | | EYS, FAM161A, FSCN2, GPR125, |
| | | | GUCA1B, HK1, HPRPF3, HGSNAT, |
| | | | IDH3B, IMPDH1, IMPG2, IFT140, |
| | | | IFT172, KLHL7, KIAA1549, KIZ, |
| | | | LRAT, MAK, MERTK, MVK, NEK2, |
| | | | NUROD1, NR2E3, NRL, OFD1, |
| | | | PDE6A, PDE6B, PDE6G, POMGNT1, |
| | | | PRCD, PROM1, PRPF3, PRPF4, |
| | | | PRPF6, PRPF8, PRPF31, PRPH2, |
| | | | RPB3, RDH12, REEP6, RP39, RGR, |
| | | | RHO, RLBP1, ROM1, RP1, RP1L1, |
| | | | RPY, RP2, RP9, RPE65, RPGR, |
| | | | SAMD11, SAG, SEMA4A, SLC7A14, |
| | | | SNRNP200, SPP2, SPATA7, TRNT1, |
| | | | TOPORS, TTC8, TULP1, USH2A, |
| | | | ZFN408, ZNF513, see also |
| | | | 20120204282 |
| Scheie syndrome (also known as | Various- liver, | | IDUA, α-L-iduronidase |
| mucopolysaccharidosis type I | spleen, eye, | | |
| S(MPS I-S)) | joint, heart, | | |
| | brain, skeletal | | |
| Schizophrenia | Brain | | Neuregulin1 (Nrg1); Erb4 (receptor for |
| | | | Neuregulin); |
| | | | Complexin1 (Cplx1); Tph1 |
| | | | Tryptophan hydroxylase; Tph2 |
| | | | Tryptophan hydroxylase 2; Neurexin |
| | | | 1; GSK3; GSK3a; |
| | | | GSK3b; 5-HTT (Slc6a4); COMT; |
| | | | DRD (Drd1a); SLC6A3; DAOA; |
| | | | DTNBP1; Dao (Dao1); TCF4; |
| | | | COL8A2 |
| Secretase Related Disorders | Various | | APH-1 (alpha and beta); PSEN1; |
| | | | NCSTN; PEN-2; Nos1, Parp1, Nat1, |
| | | | Nat2, CTSB, APP, APH1B, PSEN2, |
| | | | PSENEN, BACE1, ITM2B, CTSD, |
| | | | NOTCH1, TNF, INS, DYT10, |
| | | | ADAM17, APOE, ACE, STN, TP53, |
| | | | IL6, NGFR, IL1B, ACHE, CTNNB1, |
| | | | IGF1, IFNG, NRG1, CASP3, MAPK1, |
| | | | CDH1, APBB1, HMGCR, CREB1, |
| | | | PTGS2, HES1, CAT, TGFB1, ENO2, |
| | | | ERBB4, TRAPPC10, MAOB, NGF, |
| | | | MMP12, JAG1, CD40LG, PPARG, |
| | | | FGF2, LRP1, NOTCH4, MAPK8, |
| | | | PREP, NOTCH3, PRNP, CTSG, EGF, |
| | | | REN, CD44, SELP, GHR, ADCYAP1, |
| | | | INSR, GFAP, MMP3, MAPK10, SP1, |
| | | | MYC, CTSE, PPARA, JUN, TIMP1, |
| | | | IL5, IL1A, MMP9, HTR4, HSPG2, |
| | | | KRAS, CYCS, SMG1, IL1R1, |
| | | | PROK1, MAPK3, NTRK1, IL13, |
| | | | MME, TKT, CXCR2, CHRM1, |
| | | | ATXN1, PAWR, NOTCJ2, M6PR, |
| | | | CYP46A1, CSNK1D, MAPK14, |
| | | | PRG2, PRKCA, L1 CAM, CD40, |
| | | | NR1I2, JAG2, CTNND1, CMA1, |
| | | | SORT1, DLK1, THEM4, JUP, CD46, |
| | | | CCL11, CAV3, RNASE3, HSPA8, |
| | | | CASP9, CYP3A4, CCR3, TFAP2A, |
| | | | SCP2, CDK4, JOF1A, TCF7L2, |
| | | | B3GALTL, MDM2, RELA, CASP7, |
| | | | IDE, FANP4, CASK, ADCYAP1R1, |
| | | | ATF4, PDGFA, C21ORF33, SCG5, |
| | | | RMF123, NKFB1, ERBB2, CAV1, |
| | | | MMP7, TGFA, RXRA, STX1A, |
| | | | PSMC4, P2RY2, TNFRSF21, DLG1, |
| | | | NUMBL, SPN, PLSCR1, UBQLN2, |
| | | | UBQLN1, PCSK7, SPON1, SILV, |
| | | | QPCT, HESS, GCC1 |
| Selective IgA Deficiency | Immune system | | Type 1: MSH5; Type 2: TNFRSF13B |
| Severe Combined | Immune system | | JAK3, JAKL, DCLRE1C, ARTEMIS, |
| Immunodeficiency (SCID) and | | | SCIDA, RAG1, RAG2, ADA, PTPRC, |
| SCID-χ1, and ADA-SCID | | | CD45, LCA, IL7R, CD3D, T3D, |
| | | | IL2RG, SCIDX1, SCIDX, IMD4, |
| | | | those identified in US Pat. App. Pub. |
| | | | 20110225664, 20110091441, |
| | | | 20100229252, 20090271881 and |
| | | | 20090222937; |
| Sickle cell disease | blood | | HBB, BCL11A, BCL11Ae, cis- |
| | | | regulatory elements of the B-globin |
| | | | locus, HBG ½ promoter, HBG distal |
| | | | CCAAT box region between -92 and - |
| | | | 130 of the HBG Transcription Start |
| | | | Site, those described in |
| | | | WO2015148863, WO 2013/126794, |
| | | | US Pat. Pub. 20110182867 |
| Sly Syndrome (aka MPS VII) | | | GUSB |
| Spinocerebellar Ataxias (SCA | | | ATXN1, ATXN2, ATX3 |
| types 1, 2, 3, 6, 7, 8, 12 and 17) | | | |
| Sorsby Fundus Dystrophy | eye | | TIMP3 |
| Stargardt disease | eye | | ABCR, ELOVL4, ABCA4, PROM1 |
| Tay-Sachs Disease | Lysosomal | Various - CNS, | HEX-A |
| | Storage disease | brain, eye | |
| Thalassemia (Alpha, Beta, Delta) | blood | | HBA1, HBA2 (Alpha), HBB (Beta), |
| | | | HBB and HBD (delta), LCRB, |
| | | | BCL11A, BCL11Ae, cis-regulatory |
| | | | elements of the B-globin locus, HBG |
| | | | ½ promoter, those described in |
| | | | WO2015148860, US Pat. Pub. |
| | | | 20110182867, 2015/148860 |
| Thymic Aplasia (DiGeorge | Immune system, | | deletion of 30 to 40 genes in the |
| Syndrome; 22q11.2 deletion | thymus | | middle of chromosome 22 at |
| syndrome) | | | a location known as 22q11.2, including |
| | | | TBX1, DGCR8 |
| Transthyretin amyloidosis | liver | | TTR (transthyretin) |
| (ATTR) | | | |
| trimethylaminuria | Metabolism | | FMO3 |
| | disease | | |
| Trinucleotide Repeat Disorders | Various | | HTT; SBMA/SMAX1/AR; |
| (generally) | | | FXN/X25 ATX3; |
| | | | ATXN1; ATXN2; |
| | | | DMPK; Atrophin-1 and Atn1 |
| | | | (DRPLA Dx); CBP (Creb-BP - global |
| | | | instability); VLDLR; Atxn7; Atxn10; |
| | | | FEN1, TNRC6A, PABPN1, JPH3, |
| | | | MED15, ATXN1, ATXN3, TBP, |
| | | | CACNA1A, ATXN80S, PPP2R2B, |
| | | | ATXN7, TNRC6B, TNRC6C, CELF3, |
| | | | MAB21L1, MSH2, TMEM185A, |
| | | | SIX5, CNPY3, RAXE, GNB2, RPL14, |
| | | | ATXN8, ISR, TTR, EP400, GIGYF2, |
| | | | OGG1, STC1, CNDP1, C10ORF2, |
| | | | MAML3, DKC1, PAXIP1, CASK, |
| | | | MAPT, SP1, POLG, AFF2, THBS1, |
| | | | TP53, ESR1, CGGBP1, ABT1, KLK3, |
| | | | PRNP, JUN, KCNN3, BAX, FRAXA, |
| | | | KBTBD10, MBNL1, RAD51, |
| | | | NCOA3, ERDA1, TSC1, COMP, |
| | | | GGLC, RRAD, MSH3, DRD2, CD44, |
| | | | CTCF, CCND1, CLSPN, MEF2A, |
| | | | PTPRU, GAPDH, TRIM22, WT1, |
| | | | AHR, GPX1, TPMT, NDP, ARX, |
| | | | TYR, EGR1, UNG, NUMBL, FABP2, |
| | | | EN2, CRYGC, SRP14, CRYGB, |
| | | | PDCD1, HOXA1, ATXN2L, PMS2, |
| | | | GLA, CBL, FTH1, IL12RB2, OTX2, |
| | | | HOXA5, POLG2, DLX2, AHRR, |
| | | | MANF, RMEM158, see also |
| | | | 20110016540 |
| Turner's Syndrome (XO) | Various - | | Monosomy X |
| | reproductive | | |
| | organs, and sex | | |
| | characteristics, | | |
| | vasculature | | |
| Tuberous Sclerosis | CNS, heart, | | TSC1, TSC2 |
| | kidneys | | |
| Usher syndrome (Types I, II, and | Ears, eyes | | ABHD12, CDH23, CIB2, CLRN1, |
| III) | | | DFNB31, GPR98, HARS, MYO7A, |
| | | | PCDH15, USH1C, USH1G, USH2A, |
| | | | USH11A, those described in |
| | | | WO2015134812A1 |
| Velocardiofacial syndrome (aka | Various - | | Many genes are deleted, COM, TBX1, |
| 22q11.2 deletion syndrome, | skeletal, heart, | | and other are associated with |
| DiGeorge syndrome, conotruncal | kidney, immune | | symptoms |
| anomaly face syndrome (CTAF), | system, brain | | |
| autosomal dominant Opitz G/BB | | | |
| syndrome or Cayler cardiofacial | | | |
| syndrome) | | | |
| Von Gierke's Disease (Glycogen | Glycogen | Various - liver, | G6PC and SLC37A4 |
| Storage Disease type I) | Storage disease | kidney | |
| Von Hippel-Lindau Syndrome | Various - cell | CNS, Kidney, | VHL |
| | growth | Eye, visceral | |
| | regulation | organs | |
| | disorder | | |
| Von Willebrand Disease (Types | blood | | VWF |
| I, II and III) | | | |
| Wilson Disease | Various - | Liver, brains, | ATP7B |
| | Copper Storage | eyes, other | |
| | Disease | tissues where | |
| | | copper builds up | |
| Wiskott-Aldrich Syndrome | Immune System | | WAS |
| Xeroderma Pigmentosum | Skin | Nervous system | POLH |
| XXX Syndrome | Endocrine, brain | | X chromosome trisomy |

In some embodiments, the compositions, systems, or components thereof can be used treat or prevent a disease in a subject by modifying one or more genes associated with one or more cellular functions, such as any one or more of those in Table 4. In some embodiments, the disease is a genetic disease or disorder. In some of embodiments, the composition, system, or component thereof can modify one or more genes or polynucleotides associated with one or more genetic diseases such as any set forth in Table 4.

TABLE 4
Exemplary Genes controlling Cellular Functions

| CELLULAR FUNCTION | GENES |
|---|---|
| PI3K/AKT Signaling | PRKCE; ITGAM; ITGA5; IRAK1; PRKAA2; EIF2AK2; |
| | PTEN; EIF4E; PRKCZ; GRK6; MAPK1; TSC1; PLK1; |
| | AKT2; IKBKB; PIK3CA; CDK8; CDKN1B; NFKB2; BCL2; |
| | PIK3CB; PPP2R1A; MAPK8; BCL2L1; MAPK3; TSC2; |
| | ITGA1; KRAS; EIF4EBP1; RELA; PRKCD; NOS3; |
| | PRKAA1; MAPK9; CDK2; PPP2CA; PIM1; ITGB7; |
| | YWHAZ; ILK; TP53; RAF1; IKBKG; RELB; DYRK1A; |
| | CDKN1A; ITGB1; MAP2K2; JAK1; AKT1; JAK2; PIK3R1; |
| | CHUK; PDPK1; PPP2R5C; CTNNB1; MAP2K1; NFKB1; |
| | PAK3; ITGB3; CCND1; GSK3A; FRAP1; SFN; ITGA2; |
| | TTK; CSNK1A1; BRAF; GSK3B; AKT3; FOXO1; SGK; |
| | HSP90AA1; RPS6KB1 |
| ERK/MAPK Signaling | PRKCE; ITGAM; ITGA5; HSPB1; IRAK1; PRKAA2; |
| | EIF2AK2; RAC1; RAP1A; TLN1; EIF4E; ELK1; GRK6; |
| | MAPK1; RAC2; PLK1; AKT2; PIK3CA; CDK8; CREB1; |
| | PRKCI; PTK2; FOS; RPS6KA4; PIK3CB; PPP2R1A; |
| | PIK3C3; MAPK8; MAPK3; ITGA1; ETS1; KRAS; MYCN; |
| | EIF4EBP1; PPARG; PRKCD; PRKAA1; MAPK9; SRC; |
| | CDK2; PPP2CA; PIM1; PIK3C2A; ITGB7; YWHAZ; |
| | PPP1CC; KSR1; PXN; RAF1; FYN; DYRK1A; ITGB1; |
| | MAP2K2; PAK4; PIK3R1; STAT3; PPP2R5C; MAP2K1; |
| | PAK3; ITGB3; ESR1; ITGA2; MYC; TTK; CSNK1A1; |
| | CRKL; BRAF; ATF4; PRKCA; SRF; STAT1; SGK |
| Glucocorticoid Receptor | RAC1; TAF4B; EP300; SMAD2; TRAF6; PCAF; ELK1; |
| Signaling | MAPK1; SMAD3; AKT2; IKBKB; NCOR2; UBE2I; |
| | PIK3CA; CREB1; FOS; HSPA5; NFKB2; BCL2; |
| | MAP3K14; STAT5B; PIK3CB; PIK3C3; MAPK8; BCL2L1; |
| | MAPK3; TSC22D3; MAPK10; NRIP1; KRAS; MAPK13; |
| | RELA; STAT5A; MAPK9; NOS2A; PBX1; NR3C1; |
| | PIK3C2A; CDKN1C; TRAF2; SERPINE1; NCOA3; |
| | MAPK14; TNF; RAF1; IKBKG; MAP3K7; CREBBP; |
| | CDKN1A; MAP2K2; JAK1; IL8; NCOA2; AKT1; JAK2; |
| | PIK3R1; CHUK; STAT3; MAP2K1; NFKB1; TGFBR1; |
| | ESR1; SMAD4; CEBPB; JUN; AR; AKT3; CCL2; MMP1; |
| | STAT1; IL6; HSP90AA1 |
| Axonal Guidance Signaling | PRKCE; ITGAM; ROCK1; ITGA5; CXCR4; ADAM12; |
| | IGF1; RAC1; RAP1A; EIF4E; PRKCZ; NRP1; NTRK2; |
| | ARHGEF7; SMO; ROCK2; MAPK1; PGF; RAC2; |
| | PTPN11; GNAS; AKT2; PIK3CA; ERBB2; PRKCI; PTK2; |
| | CFL1; GNAQ; PIK3CB; CXCL12; PIK3C3; WNT11; |
| | PRKD1; GNB2L1; ABL1; MAPK3; ITGA1; KRAS; RHOA; |
| | PRKCD; PIK3C2A; ITGB7; GLI2; PXN; VASP; RAF1; |
| | FYN; ITGB1; MAP2K2; PAK4; ADAM17; AKT1; PIK3R1; |
| | GLI1; WNT5A; ADAM10; MAP2K1; PAK3; ITGB3; |
| | CDC42; VEGFA; ITGA2; EPHA8; CRKL; RND1; GSK3B; |
| | AKT3; PRKCA |
| Ephrin Receptor Signaling | PRKCE; ITGAM; ROCK1; ITGA5; CXCR4; IRAK1; |
| Actin Cytoskeleton | PRKAA2; EIF2AK2; RAC1; RAP1A; GRK6; ROCK2; |
| Signaling | MAPK1; PGF; RAC2; PTPN11; GNAS; PLK1; AKT2; |
| | DOK1; CDK8; CREB1; PTK2; CFL1; GNAQ; MAP3K14; |
| | CXCL12; MAPK8; GNB2L1; ABL1; MAPK3; ITGA1; |
| | KRAS; RHOA; PRKCD; PRKAA1; MAPK9; SRC; CDK2; |
| | PIM1; ITGB7; PXN; RAF1; FYN; DYRK1A; ITGB1; |
| | MAP2K2; PAK4; AKT1; JAK2; STAT3; ADAM10; |
| | MAP2K1; PAK3; ITGB3; CDC42; VEGFA; ITGA2; |
| | EPHA8; TTK; CSNK1A1; CRKL; BRAF; PTPN13; ATF4; |
| | AKT3; SGK |
| | ACTN4; PRKCE; ITGAM; ROCK1; ITGA5; IRAK1; |
| | PRKAA2; EIF2AK2; RAC1; INS; ARHGEF7; GRK6; |
| | ROCK2; MAPK1; RAC2; PLK1; AKT2; PIK3CA; CDK8; |
| | PTK2; CFL1; PIK3CB; MYH9; DIAPH1; PIK3C3; MAPK8; |
| | F2R; MAPK3; SLC9A1; ITGA1; KRAS; RHOA; PRKCD; |
| | PRKAA1; MAPK9; CDK2; PIM1; PIK3C2A; ITGB7; |
| | PPP1CC; PXN; VIL2; RAF1; GSN; DYRK1A; ITGB1; |
| | MAP2K2; PAK4; PIP5K1A; PIK3R1; MAP2K1; PAK3; |
| | ITGB3; CDC42; APC; ITGA2; TTK; CSNK1A1; CRKL; |
| | BRAF; VAV3; SGK |
| Huntington's Disease | PRKCE; IGF1; EP300; RCOR1; PRKCZ; HDAC4; TGM2; |
| Signaling | MAPK1; CAPNS1; AKT2; EGFR; NCOR2; SP1; CAPN2; |
| | PIK3CA; HDAC5; CREB1; PRKCI; HSPA5; REST; |
| | GNAQ; PIK3CB; PIK3C3; MAPK8; IGF1R; PRKD1; |
| | GNB2L1; BCL2L1; CAPN1; MAPK3; CASP8; HDAC2; |
| | HDAC7A; PRKCD; HDAC11; MAPK9; HDAC9; PIK3C2A; |
| | HDAC3; TP53; CASP9; CREBBP; AKT1; PIK3R1; |
| | PDPK1; CASP1; APAF1; FRAP1; CASP2; JUN; BAX; |
| | ATF4; AKT3; PRKCA; CLTC; SGK; HDAC6; CASP3 |
| Apoptosis Signaling | PRKCE; ROCK1; BID; IRAK1; PRKAA2; EIF2AK2; BAK1; |
| | BIRC4; GRK6; MAPK1; CAPNS1; PLK1; AKT2; IKBKB; |
| | CAPN2; CDK8; FAS; NFKB2; BCL2; MAP3K14; MAPK8; |
| | BCL2L1; CAPN1; MAPK3; CASP8; KRAS; RELA; |
| | PRKCD; PRKAA1; MAPK9; CDK2; PIM1; TP53; TNF; |
| | RAF1; IKBKG; RELB; CASP9; DYRK1A; MAP2K2; |
| | CHUK; APAF1; MAP2K1; NFKB1; PAK3; LMNA; CASP2; |
| | BIRC2; TTK; CSNK1A1; BRAF; BAX; PRKCA; SGK; |
| | CASP3; BIRC3; PARP1 |
| B Cell Receptor Signaling | RAC1; PTEN; LYN; ELK1; MAPK1; RAC2; PTPN11; |
| | AKT2; IKBKB; PIK3CA; CREB1; SYK; NFKB2; CAMK2A; |
| | MAP3K14; PIK3CB; PIK3C3; MAPK8; BCL2L1; ABL1; |
| | MAPK3; ETS1; KRAS; MAPK13; RELA; PTPN6; MAPK9; |
| | EGR1; PIK3C2A; BTK; MAPK14; RAF1; IKBKG; RELB; |
| | MAP3K7; MAP2K2; AKT1; PIK3R1; CHUK; MAP2K1; |
| | NFKB1; CDC42; GSK3A; FRAP1; BCL6; BCL10; JUN; |
| | GSK3B; ATF4; AKT3; VAV3; RPS6KB1 |
| Leukocyte Extravasation | ACTN4; CD44; PRKCE; ITGAM; ROCK1; CXCR4; CYBA; |
| Signaling | RAC1; RAP1A; PRKCZ; ROCK2; RAC2; PTPN11; |
| | MMP14; PIK3CA; PRKCI; PTK2; PIK3CB; CXCL12; |
| | PIK3C3; MAPK8; PRKD1; ABL1; MAPK10; CYBB; |
| | MAPK13; RHOA; PRKCD; MAPK9; SRC; PIK3C2A; BTK; |
| | MAPK14; NOX1; PXN; VIL2; VASP; ITGB1; MAP2K2; |
| | CTNND1; PIK3R1; CTNNB1; CLDN1; CDC42; F11R; ITK; |
| | CRKL; VAV3; CTTN; PRKCA; MMP1; MMP9 |
| Integrin Signaling | ACTN4; ITGAM; ROCK1; ITGA5; RAC1; PTEN; RAP1A; |
| | TLN1; ARHGEF7; MAPK1; RAC2; CAPNS1; AKT2; |
| | CAPN2; PIK3CA; PTK2; PIK3CB; PIK3C3; MAPK8; |
| | CAV1; CAPN1; ABL1; MAPK3; ITGA1; KRAS; RHOA; |
| | SRC; PIK3C2A; ITGB7; PPP1CC; ILK; PXN; VASP; |
| | RAF1; FYN; ITGB1; MAP2K2; PAK4; AKT1; PIK3R1; |
| | TNK2; MAP2K1; PAK3; ITGB3; CDC42; RND3; ITGA2; |
| | CRKL; BRAF; GSK3B; AKT3 |
| Acute Phase Response | IRAK1; SOD2; MYD88; TRAF6; ELK1; MAPK1; PTPN11; |
| Signaling | AKT2; IKBKB; PIK3CA; FOS; NFKB2; MAP3K14; |
| | PIK3CB; MAPK8; RIPK1; MAPK3; IL6ST; KRAS; |
| | MAPK13; IL6R; RELA; SOCS1; MAPK9; FTL; NR3C1; |
| | TRAF2; SERPINE1; MAPK14; TNF; RAF1; PDK1; |
| | IKBKG; RELB; MAP3K7; MAP2K2; AKT1; JAK2; PIK3R1; |
| | CHUK; STAT3; MAP2K1; NFKB1; FRAP1; CEBPB; JUN; |
| | AKT3; IL1R1; IL6 |
| PTEN Signaling | ITGAM; ITGA5; RAC1; PTEN; PRKCZ; BCL2L11; |
| | MAPK1; RAC2; AKT2; EGFR; IKBKB; CBL; PIK3CA; |
| | CDKN1B; PTK2; NFKB2; BCL2; PIK3CB; BCL2L1; |
| | MAPK3; ITGA1; KRAS; ITGB7; ILK; PDGFRB; INSR; |
| | RAF1; IKBKG; CASP9; CDKN1A; ITGB1; MAP2K2; |
| | AKT1; PIK3R1; CHUK; PDGFRA; PDPK1; MAP2K1; |
| | NFKB1; ITGB3; CDC42; CCND1; GSK3A; ITGA2; |
| | GSK3B; AKT3; FOXO1; CASP3; RPS6KB1 |
| p53 Signaling | PTEN; EP300; BBC3; PCAF; FASN; BRCA1; GADD45A; |
| Aryl Hydrocarbon Receptor | BIRC5; AKT2; PIK3CA; CHEK1; TP53INP1; BCL2; |
| Signaling | PIK3CB; PIK3C3; MAPK8; THBS1; ATR; BCL2L1; E2F1; |
| | PMAIP1; CHEK2; TNFRSF10B; TP73; RB1; HDAC9; |
| | CDK2; PIK3C2A; MAPK14; TP53; LRDD; CDKN1A; |
| | HIPK2; AKT1; PIK3R1; RRM2B; APAF1; CTNNB1; |
| | SIRT1; CCND1; PRKDC; ATM; SFN; CDKN2A; JUN; |
| | SNAI2; GSK3B; BAχ; AKT3 |
| | HSPB1; EP300; FASN; TGM2; RXRA; MAPK1; NQO1; |
| | NCOR2; SP1; ARNT; CDKN1B; FOS; CHEK1; |
| | SMARCA4; NFKB2; MAPK8; ALDH1A1; ATR; E2F1; |
| | MAPK3; NRIP1; CHEK2; RELA; TP73; GSTP1; RB1; |
| | SRC; CDK2; AHR; NFE2L2; NCOA3; TP53; TNF; |
| | CDKN1A; NCOA2; APAF1; NFKB1; CCND1; ATM; ESR1; |
| | CDKN2A; MYC; JUN; ESR2; BAX; IL6; CYP1B1; |
| | HSP90AA1 |
| Xenobiotic Metabolism | PRKCE; EP300; PRKCZ; RXRA; MAPK1; NQO1; |
| Signaling | NCOR2; PIK3CA; ARNT; PRKCI; NFKB2; CAMK2A; |
| | PIK3CB; PPP2R1A; PIK3C3; MAPK8; PRKD1; |
| | ALDH1A1; MAPK3; NRIP1; KRAS; MAPK13; PRKCD; |
| | GSTP1; MAPK9; NOS2A; ABCB1; AHR; PPP2CA; FTL; |
| | NFE2L2; PIK3C2A; PPARGC1A; MAPK14; TNF; RAF1; |
| | CREBBP; MAP2K2; PIK3R1; PPP2R5C; MAP2K1; |
| | NFKB1; KEAP1; PRKCA; EIF2AK3; IL6; CYP1B1; |
| | HSP90AA1 |
| SAPK/JNK Signaling | PRKCE; IRAK1; PRKAA2; EIF2AK2; RAC1; ELK1; |
| | GRK6; MAPK1; GADD45A; RAC2; PLK1; AKT2; PIK3CA; |
| | FADD; CDK8; PIK3CB; PIK3C3; MAPK8; RIPK1; |
| | GNB2L1; IRS1; MAPK3; MAPK10; DAXX; KRAS; |
| | PRKCD; PRKAA1; MAPK9; CDK2; PIM1; PIK3C2A; |
| | TRAF2; TP53; LCK; MAP3K7; DYRK1A; MAP2K2; |
| | PIK3R1; MAP2K1; PAK3; CDC42; JUN; TTK; CSNK1A1; |
| | CRKL; BRAF; SGK |
| PPAr/RXR Signaling | PRKAA2; EP300; INS; SMAD2; TRAF6; PPARA; FASN; |
| | RXRA; MAPK1; SMAD3; GNAS; IKBKB; NCOR2; |
| | ABCA1; GNAQ; NFKB2; MAP3K14; STAT5B; MAPK8; |
| | IRS1; MAPK3; KRAS; RELA; PRKAA1; PPARGC1A; |
| | NCOA3; MAPK14; INSR; RAF1; IKBKG; RELB; MAP3K7; |
| | CREBBP; MAP2K2; JAK2; CHUK; MAP2K1; NFKB1; |
| | TGFBR1; SMAD4; JUN; IL1R1; PRKCA; IL6; HSP90AA1; |
| | ADIPOQ |
| NF-KB Signaling | IRAK1; EIF2AK2; EP300; INS; MYD88; PRKCZ; TRAF6; |
| | TBK1; AKT2; EGFR; IKBKB; PIK3CA; BTRC; NFKB2; |
| | MAP3K14; PIK3CB; PIK3C3; MAPK8; RIPK1; HDAC2; |
| | KRAS; RELA; PIK3C2A; TRAF2; TLR4; PDGFRB; TNF; |
| | INSR; LCK; IKBKG; RELB; MAP3K7; CREBBP; AKT1; |
| | PIK3R1; CHUK; PDGFRA; NFKB1; TLR2; BCL10; |
| | GSK3B; AKT3; TNFAIP3; IL1R1 |
| Neuregulin Signaling | ERBB4; PRKCE; ITGAM; ITGA5; PTEN; PRKCZ; ELK1; |
| Wnt & Beta catenin | MAPK1; PTPN11; AKT2; EGFR; ERBB2; PRKCI; |
| Signaling | CDKN1B; STAT5B; PRKD1; MAPK3; ITGA1; KRAS; |
| | PRKCD; STAT5A; SRC; ITGB7; RAF1; ITGB1; MAP2K2; |
| | ADAM17; AKT1; PIK3R1; PDPK1; MAP2K1; ITGB3; |
| | EREG; FRAP1; PSEN1; ITGA2; MYC; NRG1; CRKL; |
| | AKT3; PRKCA; HSP90AA1; RPS6KB1 |
| | CD44; EP300; LRP6; DVL3; CSNK1E; GJA1; SMO; |
| | AKT2; PIN1; CDH1; BTRC; GNAQ; MARK2; PPP2R1A; |
| | WNT11; SRC; DKK1; PPP2CA; SOX6; SFRP2; ILK; |
| | LEF1; SOX9; TP53; MAP3K7; CREBBP; TCF7L2; AKT1; |
| | PPP2R5C; WNT5A; LRP5; CTNNB1; TGFBR1; CCND1; |
| | GSK3A; DVL1; APC; CDKN2A; MYC; CSNK1A1; GSK3B; |
| | AKT3; SOX2 |
| Insulin Receptor Signaling | PTEN; INS; EIF4E; PTPN1; PRKCZ; MAPK1; TSC1; |
| | PTPN11; AKT2; CBL; PIK3CA; PRKCI; PIK3CB; PIK3C3; |
| | MAPK8; IRS1; MAPK3; TSC2; KRAS; EIF4EBP1; |
| | SLC2A4; PIK3C2A; PPP1CC; INSR; RAF1; FYN; |
| | MAP2K2; JAK1; AKT1; JAK2; PIK3R1; PDPK1; MAP2K1; |
| | GSK3A; FRAP1; CRKL; GSK3B; AKT3; FOXO1; SGK; |
| | RPS6KB1 |
| IL-6 Signaling | HSPB1; TRAF6; MAPKAPK2; ELK1; MAPK1; PTPN11; |
| | IKBKB; FOS; NFKB2; MAP3K14; MAPK8; MAPK3; |
| | MAPK10; IL6ST; KRAS; MAPK13; IL6R; RELA; SOCS1; |
| | MAPK9; ABCB1; TRAF2; MAPK14; TNF; RAF1; IKBKG; |
| | RELB; MAP3K7; MAP2K2; IL8; JAK2; CHUK; STAT3; |
| | MAP2K1; NFKB1; CEBPB; JUN; IL1R1; SRF; IL6 |
| Hepatic Cholestasis | PRKCE; IRAK1; INS; MYD88; PRKCZ; TRAF6; PPARA; |
| | RXRA; IKBKB; PRKCI; NFKB2; MAP3K14; MAPK8; |
| | PRKD1; MAPK10; RELA; PRKCD; MAPK9; ABCB1; |
| | TRAF2; TLR4; TNF; INSR; IKBKG; RELB; MAP3K7; IL8; |
| | CHUK; NR1H2; TJP2; NFKB1; ESR1; SREBF1; FGFR4; |
| | JUN; IL1R1; PRKCA; IL6 |
| IGF-1 Signaling | IGF1; PRKCZ; ELK1; MAPK1; PTPN11; NEDD4; AKT2; |
| | PIK3CA; PRKCI; PTK2; FOS; PIK3CB; PIK3C3; MAPK8; |
| | IGF1R; IRS1; MAPK3; IGFBP7; KRAS; PIK3C2A; |
| | YWHAZ; PXN; RAF1; CASP9; MAP2K2; AKT1; PIK3R1; |
| | PDPK1; MAP2K1; IGFBP2; SFN; JUN; CYR61; AKT3; |
| | FOXO1; SRF; CTGF; RPS6KB1 |
| NRF2-mediated Oxidative | PRKCE; EP300; SOD2; PRKCZ; MAPK1; SQSTM1; |
| Stress Response | NQO1; PIK3CA; PRKCI; FOS; PIK3CB; PIK3C3; MAPK8; |
| | PRKD1; MAPK3; KRAS; PRKCD; GSTP1; MAPK9; FTL; |
| | NFE2L2; PIK3C2A; MAPK14; RAF1; MAP3K7; CREBBP; |
| | MAP2K2; AKT1; PIK3R1; MAP2K1; PPIB; JUN; KEAP1; |
| | GSK3B; ATF4; PRKCA; EIF2AK3; HSP90AA1 |
| Hepatic Fibrosis/Hepatic | EDN1; IGF1; KDR; FLT1; SMAD2; FGFR1; MET; PGF; |
| Stellate Cell Activation | SMAD3; EGFR; FAS; CSF1; NFKB2; BCL2; MYH9; |
| | IGF1R; IL6R; RELA; TLR4; PDGFRB; TNF; RELB; IL8; |
| | PDGFRA; NFKB1; TGFBR1; SMAD4; VEGFA; BAX; |
| | IL1R1; CCL2; HGF; MMP1; STAT1; IL6; CTGF; MMP9 |
| PPAR Signaling | EP300; INS; TRAF6; PPARA; RXRA; MAPK1; IKBKB; |
| | NCOR2; FOS; NFKB2; MAP3K14; STAT5B; MAPK3; |
| | NRIP1; KRAS; PPARG; RELA; STAT5A; TRAF2; |
| | PPARGC1A; PDGFRB; TNF; INSR; RAF1; IKBKG; |
| | RELB; MAP3K7; CREBBP; MAP2K2; CHUK; PDGFRA; |
| | MAP2K1; NFKB1; JUN; IL1R1; HSP90AA1 |
| Fc Epsilon RI Signaling | PRKCE; RAC1; PRKCZ; LYN; MAPK1; RAC2; PTPN11; |
| | AKT2; PIK3CA; SYK; PRKCI; PIK3CB; PIK3C3; MAPK8; |
| | PRKD1; MAPK3; MAPK10; KRAS; MAPK13; PRKCD; |
| | MAPK9; PIK3C2A; BTK; MAPK14; TNF; RAF1; FYN; |
| | MAP2K2; AKT1; PIK3R1; PDPK1; MAP2K1; AKT3; |
| | VAV3; PRKCA |
| G-Protein Coupled | PRKCE; RAP1A; RGS16; MAPK1; GNAS; AKT2; IKBKB; |
| Receptor Signaling | PIK3CA; CREB1; GNAQ; NFKB2; CAMK2A; PIK3CB; |
| | PIK3C3; MAPK3; KRAS; RELA; SRC; PIK3C2A; RAF1; |
| | IKBKG; RELB; FYN; MAP2K2; AKT1; PIK3R1; CHUK; |
| | PDPK1; STAT3; MAP2K1; NFKB1; BRAF; ATF4; AKT3; |
| | PRKCA |
| Inositol Phosphate | PRKCE; IRAK1; PRKAA2; EIF2AK2; PTEN; GRK6; |
| Metabolism | MAPK1; PLK1; AKT2; PIK3CA; CDK8; PIK3CB; PIK3C3; |
| | MAPK8; MAPK3; PRKCD; PRKAA1; MAPK9; CDK2; |
| | PIM1; PIK3C2A; DYRK1A; MAP2K2; PIP5K1A; PIK3R1; |
| | MAP2K1; PAK3; ATM; TTK; CSNK1A1; BRAF; SGK |
| PDGF Signaling | EIF2AK2; ELK1; ABL2; MAPK1; PIK3CA; FOS; PIK3CB; |
| | PIK3C3; MAPK8; CAV1; ABL1; MAPK3; KRAS; SRC; |
| | PIK3C2A; PDGFRB; RAF1; MAP2K2; JAK1; JAK2; |
| | PIK3R1; PDGFRA; STAT3; SPHK1; MAP2K1; MYC; |
| | JUN; CRKL; PRKCA; SRF; STAT1; SPHK2 |
| VEGF Signaling | ACTN4; ROCK1; KDR; FLT1; ROCK2; MAPK1; PGF; |
| | AKT2; PIK3CA; ARNT; PTK2; BCL2; PIK3CB; PIK3C3; |
| | BCL2L1; MAPK3; KRAS; HIF1A; NOS3; PIK3C2A; PXN; |
| | RAF1; MAP2K2; ELAVL1; AKT1; PIK3R1; MAP2K1; SFN; |
| | VEGFA; AKT3; FOXO1; PRKCA |
| Natural Killer Cell Signaling | PRKCE; RAC1; PRKCZ; MAPK1; RAC2; PTPN11; |
| | KIR2DL3; AKT2; PIK3CA; SYK; PRKCI; PIK3CB; |
| | PIK3C3; PRKD1; MAPK3; KRAS; PRKCD; PTPN6; |
| | PIK3C2A; LCK; RAF1; FYN; MAP2K2; PAK4; AKT1; |
| | PIK3R1; MAP2K1; PAK3; AKT3; VAV3; PRKCA |
| Cell Cycle: G1/S | HDAC4; SMAD3; SUV39H1; HDAC5; CDKN1B; BTRC; |
| Checkpoint Regulation | ATR; ABL1; E2F1; HDAC2; HDAC7A; RB1; HDAC11; |
| | HDAC9; CDK2; E2F2; HDAC3; TP53; CDKN1A; CCND1; |
| | E2F4; ATM; RBL2; SMAD4; CDKN2A; MYC; NRG1; |
| | GSK3B; RBL1; HDAC6 |
| T Cell Receptor Signaling | RAC1; ELK1; MAPK1; IKBKB; CBL; PIK3CA; FOS; |
| | NFKB2; PIK3CB; PIK3C3; MAPK8; MAPK3; KRAS; |
| | RELA; PIK3C2A; BTK; LCK; RAF1; IKBKG; RELB; FYN; |
| | MAP2K2; PIK3R1; CHUK; MAP2K1; NFKB1; ITK; BCL10; |
| | JUN; VAV3 |
| Death Receptor Signaling | CRADD; HSPB1; BID; BIRC4; TBK1; IKBKB; FADD; |
| | FAS; NFKB2; BCL2; MAP3K14; MAPK8; RIPK1; CASP8; |
| | DAXX; TNFRSF10B; RELA; TRAF2; TNF; IKBKG; RELB; |
| | CASP9; CHUK; APAF1; NFKB1; CASP2; BIRC2; CASP3; |
| | BIRC3 |
| FGF Signaling | RAC1; FGFR1; MET; MAPKAPK2; MAPK1; PTPN11; |
| | AKT2; PIK3CA; CREB1; PIK3CB; PIK3C3; MAPK8; |
| | MAPK3; MAPK13; PTPN6; PIK3C2A; MAPK14; RAF1; |
| | AKT1; PIK3R1; STAT3; MAP2K1; FGFR4; CRKL; ATF4; |
| | AKT3; PRKCA; HGF |
| GM-CSF Signaling | LYN; ELK1; MAPK1; PTPN11; AKT2; PIK3CA; CAMK2A; |
| | STAT5B; PIK3CB; PIK3C3; GNB2L1; BCL2L1; MAPK3; |
| | ETS1; KRAS; RUNX1; PIM1; PIK3C2A; RAF1; MAP2K2; |
| | AKT1; JAK2; PIK3R1; STAT3; MAP2K1; CCND1; AKT3; |
| | STAT1 |
| Amyotrophic Lateral | BID; IGF1; RAC1; BIRC4; PGF; CAPNS1; CAPN2; |
| Sclerosis Signaling | PIK3CA; BCL2; PIK3CB; PIK3C3; BCL2L1; CAPN1; |
| | PIK3C2A; TP53; CASP9; PIK3R1; RAB5A; CASP1; |
| | APAF1; VEGFA; BIRC2; BAχ; AKT3; CASP3; BIRC3 |
| JAK/Stat Signaling | PTPN1; MAPK1; PTPN11; AKT2; PIK3CA; STAT5B; |
| | PIK3CB; PIK3C3; MAPK3; KRAS; SOCS1; STAT5A; |
| | PTPN6; PIK3C2A; RAF1; CDKN1A; MAP2K2; JAK1; |
| | AKT1; JAK2; PIK3R1; STAT3; MAP2K1; FRAP1; AKT3; |
| | STAT1 |
| Nicotinate and Nicotinamide | PRKCE; IRAK1; PRKAA2; EIF2AK2; GRK6; MAPK1; |
| Metabolism | PLK1; AKT2; CDK8; MAPK8; MAPK3; PRKCD; PRKAA1; |
| | PBEF1; MAPK9; CDK2; PIM1; DYRK1A; MAP2K2; |
| | MAP2K1; PAK3; NT5E; TTK; CSNK1A1; BRAF; SGK |
| Chemokine Signaling | CXCR4; ROCK2; MAPK1; PTK2; FOS; CFL1; GNAQ; |
| | CAMK2A; CXCL12; MAPK8; MAPK3; KRAS; MAPK13; |
| | RHOA; CCR3; SRC; PPP1CC; MAPK14; NOX1; RAF1; |
| | MAP2K2; MAP2K1; JUN; CCL2; PRKCA |
| IL-2 Signaling | ELK1; MAPK1; PTPN11; AKT2; PIK3CA; SYK; FOS; |
| | STAT5B; PIK3CB; PIK3C3; MAPK8; MAPK3; KRAS; |
| | SOCS1; STAT5A; PIK3C2A; LCK; RAF1; MAP2K2; |
| | JAK1; AKT1; PIK3R1; MAP2K1; JUN; AKT3 |
| Synaptic Long Term | PRKCE; IGF1; PRKCZ; PRDX6; LYN; MAPK1; GNAS; |
| Depression | PRKCI; GNAQ; PPP2R1A; IGF1R; PRKD1; MAPK3; |
| | KRAS; GRN; PRKCD; NOS3; NOS2A; PPP2CA; |
| | YWHAZ; RAF1; MAP2K2; PPP2R5C; MAP2K1; PRKCA |
| Estrogen Receptor | TAF4B; EP300; CARM1; PCAF; MAPK1; NCOR2; |
| Signaling | SMARCA4; MAPK3; NRIP1; KRAS; SRC; NR3C1; |
| | HDAC3; PPARGC1A; RBM9; NCOA3; RAF1; CREBBP; |
| | MAP2K2; NCOA2; MAP2K1; PRKDC; ESR1; ESR2 |
| Protein Ubiquitination | TRAF6; SMURF1; BIRC4; BRCA1; UCHL1; NEDD4; |
| Pathway | CBL; UBE2I; BTRC; HSPA5; USP7; USP10; FBXW7; |
| | USP9X; STUB1; USP22; B2M; BIRC2; PARK2; USP8; |
| | USP1; VHL; HSP90AA1; BIRC3 |
| IL-10 Signaling | TRAF6; CCR1; ELK1; IKBKB; SP1; FOS; NFKB2; |
| | MAP3K14; MAPK8; MAPK13; RELA; MAPK14; TNF; |
| | IKBKG; RELB; MAP3K7; JAK1; CHUK; STAT3; NFKB1; |
| | JUN; IL1R1; IL6 |
| VDR/RXR Activation | PRKCE; EP300; PRKCZ; RXRA; GADD45A; HES1; |
| | NCOR2; SP1; PRKCI; CDKN1B; PRKD1; PRKCD; |
| | RUNX2; KLF4; YY1; NCOA3; CDKN1A; NCOA2; SPP1; |
| | LRP5; CEBPB; FOXO1; PRKCA |
| TGF-beta Signaling | EP300; SMAD2; SMURF1; MAPK1; SMAD3; SMAD1; |
| | FOS; MAPK8; MAPK3; KRAS; MAPK9; RUNX2; |
| | SERPINE1; RAF1; MAP3K7; CREBBP; MAP2K2; |
| | MAP2K1; TGFBR1; SMAD4; JUN; SMAD5 |
| Toll-like Receptor Signaling | IRAK1; EIF2AK2; MYD88; TRAF6; PPARA; ELK1; |
| | IKBKB; FOS; NFKB2; MAP3K14; MAPK8; MAPK13; |
| | RELA; TLR4; MAPK14; IKBKG; RELB; MAP3K7; CHUK; |
| | NFKB1; TLR2; JUN |
| p38 MAPK Signaling | HSPB1; IRAK1; TRAF6; MAPKAPK2; ELK1; FADD; FAS; |
| | CREB1; DDIT3; RPS6KA4; DAXX; MAPK13; TRAF2; |
| | MAPK14; TNF; MAP3K7; TGFBR1; MYC; ATF4; IL1R1; |
| | SRF; STAT1 |
| Neurotrophin/TRK Signaling | NTRK2; MAPK1; PTPN11; PIK3CA; CREB1; FOS; |
| | PIK3CB; PIK3C3; MAPK8; MAPK3; KRAS; PIK3C2A; |
| | RAF1; MAP2K2; AKT1; PIK3R1; PDPK1; MAP2K1; |
| | CDC42; JUN; ATF4 |
| FXR/RXR Activation | INS; PPARA; FASN; RXRA; AKT2; SDC1; MAPK8; |
| | APOB; MAPK10; PPARG; MTTP; MAPK9; PPARGC1A; |
| | TNF; CREBBP; AKT1; SREBF1; FGFR4; AKT3; FOXO1 |
| Synaptic Long Term | PRKCE; RAP1A; EP300; PRKCZ; MAPK1; CREB1; |
| Potentiation | PRKCI; GNAQ; CAMK2A; PRKD1; MAPK3; KRAS; |
| | PRKCD; PPP1CC; RAF1; CREBBP; MAP2K2; MAP2K1; |
| | ATF4; PRKCA |
| Calcium Signaling | RAP1A; EP300; HDAC4; MAPK1; HDAC5; CREB1; |
| | CAMK2A; MYH9; MAPK3; HDAC2; HDAC7A; HDAC11; |
| | HDAC9; HDAC3; CREBBP; CALR; CAMKK2; ATF4; |
| | HDAC6 |
| EGF Signaling | ELK1; MAPK1; EGFR; PIK3CA; FOS; PIK3CB; PIK3C3; |
| | MAPK8; MAPK3; PIK3C2A; RAF1; JAK1; PIK3R1; |
| | STAT3; MAP2K1; JUN; PRKCA; SRF; STAT1 |
| Hypoxia Signaling in the | EDN1; PTEN; EP300; NQO1; UBE2I; CREB1; ARNT; |
| Cardiovascular System | HIF1A; SLC2A4; NOS3; TP53; LDHA; AKT1; ATM; |
| | VEGFA; JUN; ATF4; VHL; HSP90AA1 |
| LPS/IL-1 Mediated Inhibition | IRAK1; MYD88; TRAF6; PPARA; RXRA; ABCA1; |
| of RXR Function | MAPK8; ALDH1A1; GSTP1; MAPK9; ABCB1; TRAF2; |
| | TLR4; TNF; MAP3K7; NR1H2; SREBF1; JUN; IL1R1 |
| LXR/RXR Activation | FASN; RXRA; NCOR2; ABCA1; NFKB2; IRF3; RELA; |
| | NOS2A; TLR4; TNF; RELB; LDLR; NR1H2; NFKB1; |
| | SREBF1; IL1R1; CCL2; IL6; MMP9 |
| Amyloid Processing | PRKCE; CSNK1E; MAPK1; CAPNS1; AKT2; CAPN2; |
| | CAPN1; MAPK3; MAPK13; MAPT; MAPK14; AKT1; |
| | PSEN1; CSNK1A1; GSK3B; AKT3; APP |
| IL-4 Signaling | AKT2; PIK3CA; PIK3CB; PIK3C3; IRS1; KRAS; SOCS1; |
| | PTPN6; NR3C1; PIK3C2A; JAK1; AKT1; JAK2; PIK3R1; |
| | FRAP1; AKT3; RPS6KB1 |
| Cell Cycle: G2/M DNA | EP300; PCAF; BRCA1; GADD45A; PLK1; BTRC; |
| Damage Checkpoint | CHEK1; ATR; CHEK2; YWHAZ; TP53; CDKN1A; |
| Regulation | PRKDC; ATM; SFN; CDKN2A |
| Nitric Oxide Signaling in the | KDR; FLT1; PGF; AKT2; PIK3CA; PIK3CB; PIK3C3; |
| Cardiovascular System | CAV1; PRKCD; NOS3; PIK3C2A; AKT1; PIK3R1; |
| | VEGFA; AKT3; HSP90AA1 |
| Purine Metabolism | NME2; SMARCA4; MYH9; RRM2; ADAR; EIF2AK4; |
| | PKM2; ENTPD1; RAD51; RRM2B; TJP2; RAD51C; |
| | NT5E; POLD1; NME1 |
| cAMP-mediated Signaling | RAP1A; MAPK1; GNAS; CREB1; CAMK2A; MAPK3; |
| | SRC; RAF1; MAP2K2; STAT3; MAP2K1; BRAF; ATF4 |
| Mitochondrial Dysfunction | SOD2; MAPK8; CASP8; MAPK10; MAPK9; CASP9; |
| Notch Signaling | PARK7; PSEN1; PARK2; APP; CASP3 |
| | HES1; JAG1; NUMB; NOTCH4; ADAM17; NOTCH2; |
| | PSEN1; NOTCH3; NOTCH1; DLL4 |
| Endoplasmic Reticulum | HSPA5; MAPK8; XBP1; TRAF2; ATF6; CASP9; ATF4; |
| Stress Pathway | EIF2AK3; CASP3 |
| Pyrimidine Metabolism | NME2; AICDA; RRM2; EIF2AK4; ENTPD1; RRM2B; |
| | NT5E; POLD1; NME1 |
| Parkinson's Signaling | UCHL1; MAPK8; MAPK13; MAPK14; CASP9; PARK7; |
| | PARK2; CASP3 |
| Cardiac & Beta Adrenergic | GNAS; GNAQ; PPP2R1A; GNB2L1; PPP2CA; PPP1CC; |
| Signaling | PPP2R5C |
| Glycolysis/Gluconeogenesis | HK2; GCK; GPI; ALDH1A1; PKM2; LDHA; HK1 |
| Interferon Signaling | IRF1; SOCS1; JAK1; JAK2; IFITM1; STAT1; IFIT3 |
| Sonic Hedgehog Signaling | ARRB2; SMO; GLI2; DYRK1A; GLI1; GSK3B; DYRK1B |
| Glycerophospholipid | PLD1; GRN; GPAM; YWHAZ; SPHK1; SPHK2 |
| Metabolism | |
| Phospholipid Degradation | PRDX6; PLD1; GRN; YWHAZ; SPHK1; SPHK2 |
| Tryptophan Metabolism | SIAH2; PRMT5; NEDD4; ALDH1A1; CYP1B1; SIAH1 |
| Lysine Degradation | SUV39H1; EHMT2; NSD1; SETD7; PPP2R5C |
| Nucleotide Excision Repair | ERCC5; ERCC4; XPA; XPC; ERCC1 |
| Pathway | |
| Starch and Sucrose | UCHL1; HK2; GCK; GPI; HK1 |
| Metabolism | |
| Aminosugars Metabolism | NQO1; HK2; GCK; HK1 |
| Arachidonic Acid | PRDX6; GRN; YWHAZ; CYP1B1 |
| Metabolism | |
| Circadian Rhythm Signaling | CSNK1E; CREB1; ATF4; NR1D1 |
| Coagulation System | BDKRB1; F2R; SERPINE1; F3 |
| Dopamine Receptor | PPP2R1A; PPP2CA; PPP1CC; PPP2R5C |
| Signaling | |
| Glutathione Metabolism | IDH2; GSTP1; ANPEP; IDH1 |
| Glycerolipid Metabolism | ALDH1A1; GPAM; SPHK1; SPHK2 |
| Linoleic Acid Metabolism | PRDX6; GRN; YWHAZ; CYP1B1 |
| Methionine Metabolism | DNMT1; DNMT3B; AHCY; DNMT3A |
| Pyruvate Metabolism | GLO1; ALDH1A1; PKM2; LDHA |
| Arginine and Proline | ALDH1A1; NOS3; NOS2A |
| Metabolism | |
| Eicosanoid Signaling | PRDX6; GRN; YWHAZ |
| Fructose and Mannose | HK2; GCK; HK1 |
| Metabolism | |
| Galactose Metabolism | HK2; GCK; HK1 |
| Stilbene, Coumarine and | PRDX6; PRDX1; TYR |
| Lignin Biosynthesis | |
| Antigen Presentation | CALR; B2M |
| Pathway | |
| Biosynthesis of Steroids | NQO1; DHCR7 |
| Butanoate Metabolism | ALDH1A1; NLGN1 |
| Citrate Cycle | IDH2; IDH1 |
| Fatty Acid Metabolism | ALDH1A1; CYP1B1 |
| Glycerophospholipid | PRDX6; CHKA |
| Metabolism | |
| Histidine Metabolism | PRMT5; ALDH1A1 |
| Inositol Metabolism | ERO1L; APEX1 |
| Metabolism of Xenobiotics | GSTP1; CYP1B1 |
| by Cytochrome p450 | |
| Methane Metabolism | PRDX6; PRDX1 |
| Phenylalanine Metabolism | PRDX6; PRDX1 |
| Propanoate Metabolism | ALDH1A1; LDHA |
| Selenoamino Acid | PRMT5; AHCY |
| Metabolism | |
| Sphingolipid Metabolism | SPHK1; SPHK2 |
| Aminophosphonate | PRMT5 |
| Metabolism | |
| Androgen and Estrogen | PRMT5 |
| Metabolism | |
| Ascorbate and Aldarate | ALDH1A1 |
| Metabolism | |
| Bile Acid Biosynthesis | ALDH1A1 |
| Cysteine Metabolism | LDHA |
| Fatty Acid Biosynthesis | FASN |
| Glutamate Receptor | GNB2L1 |
| Signaling | |
| NRF2-mediated Oxidative | PRDX1 |
| Stress Response | |
| Pentose Phosphate | GPI |
| Pathway | |
| Pentose and Glucuronate | UCHL1 |
| Interconversions | |
| Retinol Metabolism | ALDH1A1 |
| Riboflavin Metabolism | TYR |
| Tyrosine Metabolism | PRMT5, TYR |
| Ubiquinone Biosynthesis | PRMT5 |
| Valine, Leucine and | ALDH1A1 |
| Isoleucine Degradation | |
| Glycine, Serine and | CHKA |
| Threonine Metabolism | |
| Lysine Degradation | ALDH1A1 |
| Pain/Taste | TRPM5; TRPA1 |
| Pain | TRPM7; TRPC5; TRPC6; TRPC1; Cnr1; crn2; Grk2; |
| | Trpa1; Pomc; Cgrp; Crf; Pka; Era; Nr2b; TRPM5; Prkaca; |
| | Prkacb; Prkar1a; Prkar2a |
| Mitochondrial Function | AIF; CytC; SMAC (Diablo); Aifm-1; Aifm-2 |
| Developmental Neurology | BMP-4; Chordin (Chrd); Noggin (Nog); WNT (Wnt2; |
| | Wnt2b; Wnt3a; Wnt4; Wnt5a; Wnt6; Wnt7b; Wnt8b; |
| | Wnt9a; Wnt9b; Wnt10a; Wnt10b; Wnt16); beta-catenin; |
| | Dkk-1; Frizzled related proteins; Otx-2; Gbx2; FGF-8; |
| | Reelin; Dab1; unc-86 (Pou4f1 or Brn3a); Numb; Reln |

In an aspect, the invention provides a method of individualized or personalized treatment of a genetic disease in a subject in need of such treatment comprising: (a) introducing one or more mutations ex vivo in a tissue, organ or a cell line, or in vivo in a transgenic non-human mammal, comprising delivering to cell(s) of the tissue, organ, cell or mammal a composition comprising the particle delivery system or the delivery system or the virus particle of any one of the above embodiment or the cell of any one of the above embodiment, wherein the specific mutations or precise sequence substitutions are or have been correlated to the genetic disease; (b) testing treatment(s) for the genetic disease on the cells to which the vector has been delivered that have the specific mutations or precise sequence substitutions correlated to the genetic disease; and (c) treating the subject based on results from the testing of treatment(s) of step (b).

### Infectious Diseases

In some embodiments, the composition, system(s), or component(s) thereof can be used to diagnose, prognose, treat, and/or prevent an infectious disease caused by a microorganism, such as bacteria, virus, fungi, parasites, or combinations thereof.

In some embodiments, the system(s) or component(s) thereof can be capable of targeting specific microorganism within a mixed population. Exemplary methods of such techniques are described in e.g. Gomaa A A, Klumpe H E, Luo M L, Selle K, Barrangou R, Beisel

CL. 2014. Programmable removal of bacterial strains by use of genome-targeting composition, systems. mBio 5: e00928-13; Citorik R J, Mimee M, Lu T K. 2014. Sequence-specific antimicrobials using efficiently delivered RNA-guided nucleases. Nat Biotechnol 32:1141-1145, the teachings of which can be adapted for use with the compositions, systems, and components thereof described herein.

In some embodiments, the composition, system(s), and/or components thereof can be capable of targeting pathogenic and/or drug-resistant microorganisms, such as bacteria, virus, parasites, and fungi. In some embodiments, the composition, system(s), and/or components thereof can be capable of targeting and modifying one or more polynucleotides in a pathogenic microorganism such that the microorganism is less virulent, killed, inhibited, or is otherwise rendered incapable of causing disease and/or infecting and/or replicating in a host cell.

In some embodiments, the pathogenic bacteria that can be targeted and/or modified by the composition, system(s), and/or component(s) thereof described herein include, but are not limited to, those of the genus Actinomyces (e.g., A. israelii), Bacillus (e.g., B. anthracis, B. cereus), Bactereoides (e.g., B. fragilis), Bartonella (B. henselae, B. quintana), Bordetella (B. pertussis), Borrelia (e.g., B. burgdorferi, B. garinii, B. afzelii, and B. recurreentis), Brucella (e.g., B. abortus, B. canis, B. melitensis, and B. suis), Campylobacter (e.g., C. jejuni), Chlamydia (e.g., C. pneumoniae and C. trachomatis), Chlamydophila (e.g., C. psittaci), Clostridium (e.g., C. botulinum, C. difficile, C. perfringens. C. tetani), Corynebacterium (e.g., C. diptheriae), Enterococcus (e.g., E. Faecalis, E. faecium), Ehrlichia (E. canis and E. chaffensis) Escherichia (e.g., E. coli), Francisella (e.g., F. tularensis), Haemophilus (e.g., H. influenzae), Helicobacter (H. pylori), Klebsiella (e.g., K. pneumoniae), Legionella (e.g., L. pneumophila), Leptospira (e.g., L. interrogans, L. santarosai, L. weilii, L. noguchii), Listereia (e.g., L. monocytogeenes), Mycobacterium (e.g., M. leprae, M. tuberculosis, M. ulcerans), Mycoplasma (M. pneumoniae), Neisseria (N. gonorrhoeae and N. menigitidis), Nocardia (e.g., N. asteeroides), Pseudomonas (P. aeruginosa), Rickettsia (R. rickettsia), Salmonella (S. typhi and S. typhimurium), Shigella (S. sonnei and S. dysenteriae), Staphylococcus (S. aureus, S. epidermidis, and S. saprophyticus), Streptococcus (S. agalactiaee, S. pneumoniae, S. pyogenes), Treponema (T. pallidum), Ureeaplasma (e.g., U. urealyticum), Vibrio (e.g., V. cholerae), Yersinia (e.g., Y pestis, Y, enteerocolitica, and Y, pseudotuberculosis).

In some embodiments, the pathogenic virus that can be targeted and/or modified by the composition, system(s), and/or component(s) thereof described herein include, but are not limited to, a double-stranded DNA virus, a partly double-stranded DNA virus, a single-stranded DNA virus, a positive single-stranded RNA virus, a negative single-stranded RNA virus, or a double stranded RNA virus. In some embodiments, the pathogenic virus can be from the family Adenoviridae (e.g., Adenovirus), Herpesviridae (e.g., Herpes simplex, type 1, Herpes simplex, type 2, Varicella-zoster virus, Epstein-Barr virus, Human cytomegalovirus, Human herpesvirus, type 8), Papillomaviridae (e.g., Human papillomavirus), Polyomaviridae (e.g., BK virus, JC virus), Poxviridae (e.g., smallpox), Hepadnaviridae (e.g., Hepatitis B), Parvoviridae (e.g., Parvovirus B19), Astroviridae (e.g., Human astrovirus), Caliciviridae (e.g., Norwalk virus), Picornaviridae (e.g., coxsackievirus, hepatitis A virus, poliovirus, rhinovirus), Coronaviridae (e.g., Severe acute respiratory syndrome-related coronavirus, strains: Severe acute respiratory syndrome virus, Severe acute respiratory syndrome coronavirus 2 (COVID-19)), Flaviviridae (e.g., Hepatitis C virus, yellow fever virus, dengue virus, West Nile virus, TBE virus), Togaviridae (e.g., Rubella virus), Hepeviridae (e.g., Hepatitis E virus), Retroviridae (Human immunodeficiency virus (HIV)), Orthomyxoviridae (e.g., Influenza virus), Arenaviridae (e.g., Lassa virus), Bunyaviridae (e.g., Crimean-Congo hemorrhagic fever virus, Hantaan virus), Filoviridae (e.g., Ebola virus and Marburg virus), Paramyxoviridae (e.g., Measles virus, Mumps virus, Parainfluenza virus, Respiratory syncytial virus), Rhabdoviridae (Rabies virus), Hepatitis D virus, Reoviridae (e.g., Rotavirus, Orbivirus, Coltivirus, Banna virus).

In some embodiments, the pathogenic fungi that can be targeted and/or modified by the composition, system(s), and/or component(s) thereof described herein include, but are not limited to, those of the genus Candida (e.g., C. albicans), Aspergillus (e.g., A. fumigatus, A. flavus, A. clavatus), Cryptococcus (e.g., C. neoformans, C. gattii), Histoplasma (H. capsulatum), Pneumocystis (e.g., P. jiroveecii), Stachybotrys (e.g., S. chartarum).

In some embodiments, the pathogenic parasites that can be targeted and/or modified by the composition, system(s), and/or component(s) thereof described herein include, but are not limited to, protozoa, helminths, and ectoparasites. In some embodiments, the pathogenic protozoa that can be targeted and/or modified by the composition, system(s), and/or component(s) thereof described herein include, but are not limited to, those from the groups Sarcodina (e.g., ameba such as Entamoeba), Mastigophora (e.g., flagellates such as Giardia and Leishmania), Cilophora (e.g., ciliates such as Balantidum), and sporozoa (e.g., plasmodium and cryptosporidium). In some embodiments, the pathogenic helminths that can be targeted and/or modified by the composition, system(s), and/or component(s) thereof described herein include, but are not limited to, flatworms (platyhelminths), thorny-headed worms (acanthoceephalins), and roundworms (nematodes). In some embodiments, the pathogenic ectoparasites that can be targeted and/or modified by the composition, system(s), and/or component(s) thereof described herein include, but are not limited to, ticks, fleas, lice, and mites.

In some embodiments, the pathogenic parasite that can be targeted and/or modified by the composition, system(s), and/or component(s) thereof described herein include, but are not limited to, Acanthamoeba spp., Balamuthia mandrillaris, Babesiosis spp. (e.g., Babesia B. divergens, B. bigemina, B. equi, B. microfti, B. duncani), Balantidiasis spp. (e.g., Balantidium coli), Blastocystis spp., Cryptosporidium spp., Cyclosporiasis spp. (e.g., Cyclospora cayetanensis), Dientamoebiasis spp. (e.g., Dientamoeba fragilis), Amoebiasis spp. (e.g., Entamoeba histolytica), Giardiasis spp. (e.g., Giardia lamblia), Isosporiasis spp. (e.g., Isospora belli), Leishmania spp., Naegleria spp. (e.g., Naegleria fowleri), Plasmodium spp. (e.g., Plasmodium falciparum, Plasmodium vivax, Plasmodium ovale curtisi, Plasmodium ovale wallikeri, Plasmodium malariae, Plasmodium knowlesi), Rhinosporidiosis spp. (e.g., Rhinosporidium seeberi), Sarcocystosis spp. (e.g., Sarcocystis bovihominis, Sarcocystis suihominis), Toxoplasma spp. (e.g., Toxoplasma gondii), Trichomonas spp. (e.g., Trichomonas vaginalis), Trypanosoma spp. (e.g., Trypanosoma brucei), Trypanosoma spp. (e.g., Trypanosoma cruzi), Tapeworm (e.g., Cestoda, Taenia multiceps, Taenia saginata, Taenia solium), Diphyllobothrium latum spp., Echinococcus spp. (e.g., Echinococcus granulosus, Echinococcus multilocularis, E. vogeli, E. oligarthrus), Hymenolepis spp. (e.g., Hymenolepis nana, Hymenolepis diminuta), Bertiella spp. (e.g., Bertiella mucronata, Bertiella studeri), Spirometra (e.g., Spirometra erinaceieuropaei), Clonorchis spp. (e.g., Clonorchis sinensis; Clonorchis viverrini), Dicrocoelium spp. (e.g., Dicrocoelium dendriticum), Fasciola spp. (e.g., Fasciola hepatica, Fasciola gigantica), Fasciolopsis spp. (e.g., Fasciolopsis buski), Metagonimus spp. (e.g., Metagonimus yokogawai), Metorchis spp. (e.g., Metorchis conjunctus), Opisthorchis spp. (e.g., Opisthorchis viverrini, Opisthorchis felineus), Clonorchis spp. (e.g., Clonorchis sinensis), Paragonimus spp. (e.g., Paragonimus westermani; Paragonimus africanus; Paragonimus caliensis; Paragonimus kellicotti; Paragonimus skrjabini; Paragonimus uterobilateralis), Schistosoma sp., Schistosoma spp. (e.g., Schistosoma mansoni, Schistosoma haematobium, Schistosoma japonicum, Schistosoma mekongi, and Schistosoma intercalatum), Echinostoma spp. (e.g., E. echinatum), Trichobilharzia spp. (e.g., Trichobilharzia regent), Ancylostoma spp. (e.g., Ancylostoma duodenale), Necator spp. (e.g., Necator americanus), Angiostrongylus spp., Anisakis spp., Ascaris spp. (e.g., Ascaris lumbricoides), Baylisascaris spp. (e.g., Baylisascaris procyonis), Brugia spp. (e.g., Brugia malayi, Brugia timori), Dioctophyme spp. (e.g., Dioctophyme renale), Dracunculus spp. (e.g., Dracunculus medinensis), Enterobius spp. (e.g., Enterobius vermicularis, Enterobius gregorii), Gnathostoma spp. (e.g., Gnathostoma spinigerum, Gnathostoma hispidum), Halicephalobus spp. (e.g., Halicephalobus gingivalis), Loa loa spp. (e.g., Loa loa filaria), Mansonella spp. (e.g., Mansonella streptocerca), Onchocerca spp. (e.g., Onchocerca volvulus), Strongyloides spp. (e.g., Strongyloides stercoralis), Thelazia spp. (e.g., Thelazia californiensis, Thelazia callipaeda), Toxocara spp. (e.g., Toxocara canis, Toxocara cati, Toxascaris leonine), Trichinella spp. (e.g., Trichinella spiralis, Trichinella britovi, Trichinella nelsoni, Trichinella nativa), Trichuris spp. (e.g., Trichuris trichiura, Trichuris vulpis), Wuchereria spp. (e.g., Wuchereria bancrofti), Dermatobia spp. (e.g., Dermatobia hominis), Tunga spp. (e.g., Tunga penetrans), Cochliomyia spp. (e.g., Cochliomyia hominivorax), Linguatula spp. (e.g., Linguatula serrata), Archiacanthocephala sp., Moniliformis sp. (e.g., Moniliformis moniliformis), Pediculus spp. (e.g., Pediculus humanus capitis, Pediculus humanus humanus), Pthirus spp. (e.g., Pthirus pubis), Arachnida spp. (e.g., Trombiculidae, Ixodidae, Argaside), Siphonaptera spp (e.g., Siphonaptera: Pulicinae), Cimicidae spp. (e.g., Cimex lectularius and Cimex hemipterus), Diptera spp., Demodex spp. (e.g., Demodex folliculorum/brevis/canis), Sarcoptes spp. (e.g., Sarcoptes scabiei), Dermanyssus spp. (e.g., Dermanyssus gallinae), Ornithonyssus spp. (e.g., Ornithonyssus sylviarum, Ornithonyssus bursa, Ornithonyssus bacoti), Laelaps spp. (e.g., Laelaps echidnina), Liponyssoides spp. (e.g., Liponyssoides sanguineus).

In some embodiments, the gene targets can be any of those as set forth in Table 1 of Strich and Chertow. 2019. J. Clin. Microbio. 57:4e01307-18, which is incorporated herein as if expressed in its entirety herein.

In some embodiments, the method can include delivering a composition, system, and/or component thereof to a pathogenic organism described herein, allowing the composition, system, and/or component thereof to specifically bind and modify one or more targets in the pathogenic organism, whereby the modification kills, inhibits, reduces the pathogenicity of the pathogenic organism, or otherwise renders the pathogenic organism non-pathogenic. In some embodiments, delivery of the composition, system, occurs in vivo (i.e., in the subject being treated). In some embodiments occurs by an intermediary, such as microorganism or phage that is non-pathogenic to the subject but is capable of transferring polynucleotides and/or infecting the pathogenic microorganism. In some embodiments, the intermediary microorganism can be an engineered bacteria, virus, or phage that contains the composition, system(s), and/or component(s) thereof and/or CRISPR-Cas vectors and/or vector systems. The method can include administering an intermediary microorganism containing the composition, system(s), and/or component(s) thereof and/or CRISPR-Cas vectors and/or vector systems to the subject to be treated. The intermediary microorganism can then produce the CRISPR-system and/or component thereof or transfer a composition, system, polynucleotide to the pathogenic organism. In embodiments, where the CRISPR-system and/or component thereof, vector, or vector system is transferred to the pathogenic microorganism, the composition, system, or component thereof is then produced in the pathogenic microorganism and modifies the pathogenic microorganism such that it is less virulent, killed, inhibited, or is otherwise rendered incapable of causing disease and/or infecting and/or replicating in a host or cell thereof.

In some embodiments, where the pathogenic microorganism inserts its genetic material into the host cell's genome (e.g., a virus), the composition, system can be designed such that it modifies the host cell's genome such that the viral DNA or cDNA cannot be replicated by the host cell's machinery into a functional virus. In some embodiments, where the pathogenic microorganism inserts its genetic material into the host cell's genome (e.g., a virus), the composition, system, can be designed such that it modifies the host cell's genome such that the viral DNA or cDNA is deleted from the host cell's genome.

It will be appreciated that inhibiting or killing the pathogenic microorganism, the disease and/or condition that its infection causes in the subject can be treated or prevented. Thus, also provided herein are methods of treating and/or preventing one or more diseases or symptoms thereof caused by any one or more pathogenic microorganisms, such as any of those described herein.

### Mitochondrial Diseases

Some of the most challenging mitochondrial disorders arise from mutations in mitochondrial DNA (mtDNA), a high copy number genome that is maternally inherited. In some embodiments, mtDNA mutations can be modified using a composition, system, described herein. In some embodiments, the mitochondrial disease that can be diagnosed, prognosed, treated, and/or prevented can be MELAS (mitochondrial myopathy encephalopathy, and lactic acidosis and stroke-like episodes), CPEO/PEO (chronic progressive external ophthalmoplegia syndrome/progressive external ophthalmoplegia), KSS (Kearns-Sayre syndrome), MIDD (maternally inherited diabetes and deafness), MERRF (myoclonic epilepsy associated with ragged red fibers), NIDDM (noninsulin-dependent diabetes mellitus), LHON (Leber hereditary optic neuropathy), LS (Leigh Syndrome) an aminoglycoside induced hearing disorder, NARP (neuropathy, ataxia, and pigmentary retinopathy), Extrapyramidal disorder with akinesia-rigidity, psychosis and SNHL, Nonsyndromic hearing loss a cardiomyopathy, an encephalomyopathy, Pearson's syndrome, or a combination thereof.

In some embodiments, the mtDNA of a subject can be modified in vivo or ex vivo. In some embodiments, where the mtDNA is modified ex vivo, after modification the cells containing the modified mitochondria can be administered back to the subject. In some embodiments, the composition, system, or component thereof can be capable of correcting an mtDNA mutation, or a combination thereof.

In some embodiments, at least one of the one or more mtDNA mutations is selected from the group consisting of: A3243G, C3256T, T3271C, G1019A, A1304T, A15533G, C1494T, C4467A, T1658C, G12315A, A3421G, A8344G, T8356C, G8363A, A13042T, T3200C, G3242A, A3252G, T3264C, G3316A, T3394C, T14577C, A4833G, G3460A, G9804A, G11778A, G14459A, A14484G, G15257A, T8993C, T8993G, G10197A, G13513A, T1095C, C1494T, A1555G, G1541A, C1634T, A3260G, A4269G, T7587C, A8296G, A8348G, G8363A, T9957C, T9997C, G12192A, C12297T, A14484G, G15059A, duplication of CCCCCTCCCC-tandem (SEQ ID NO: 68) repeats at positions 305-314 and/or 956-965, deletion at positions from 8,469-13,447, 4,308-14,874, and/or 4,398-14,822, 961ins/delC, the mitochondrial common deletion (e.g., mtDNA 4,977 bp deletion), and combinations thereof.

In some embodiments, the mitochondrial mutation can be any mutation as set forth in or as identified by use of one or more bioinformatic tools available at Mitomap available at mitomap.org. Such tools include, but are not limited to, “Variant Search, aka Market Finder”, Find Sequences for Any Haplogroup, aka “Sequence Finder”, “Variant Info”, “POLG Pathogenicity Prediction Server”, “MITOMASTER”, “Allele Search”, “Sequence and Variant Downloads”, “Data Downloads”. MitoMap contains reports of mutations in mtDNA that can be associated with disease and maintains a database of reported mitochondrial DNA Base Substitution Diseases: rRNA/tRNA mutations.

In some embodiments, the method includes delivering a composition, system, and/or a component thereof to a cell, and more specifically one or more mitochondria in a cell, allowing the composition, system, and/or component thereof to modify one or more target polynucleotides in the cell, and more specifically one or more mitochondria in the cell. The target polynucleotides can correspond to a mutation in the mtDNA, such as any one or more of those described herein. In some embodiments, the modification can alter a function of the mitochondria such that the mitochondria functions normally or at least is/are less dysfunctional as compared to an unmodified mitochondria. Modification can occur in vivo or ex vivo. Where modification is performed ex vivo, cells containing modified mitochondria can be administered to a subject in need thereof in an autologous or allogenic manner.

### Kits

Any of the compounds, compositions, formulations, particles, cells, described herein or a combination thereof can be presented as a combination kit. As used herein, the terms “combination kit” or “kit of parts” refers to the compounds, compositions, formulations, particles, cells and any additional components that are used to package, sell, market, deliver, and/or administer the combination of elements or a single element, such as the active ingredient, contained therein. Such additional components include, but are not limited to, packaging, syringes, blister packages, bottles, and the like. When one or more of the compounds, compositions, formulations, particles, cells, described herein or a combination thereof (e.g., agents) contained in the kit are administered simultaneously, the combination kit can contain the active agents in a single formulation, such as a pharmaceutical formulation, (e.g., a tablet) or in separate formulations. When the compounds, compositions, formulations, particles, and cells described herein or a combination thereof and/or kit components are not administered simultaneously, the combination kit can contain each agent or other component in separate pharmaceutical formulations. The separate kit components can be contained in a single package or in separate packages within the kit.

In some embodiments, the combination kit also includes instructions printed on or otherwise contained in a tangible medium of expression. The instructions can provide information regarding the content of the compounds, compositions, formulations, particles, cells, described herein or a combination thereof contained therein, safety information regarding the content of the compounds, compositions, formulations (e.g., pharmaceutical formulations), particles, and cells described herein or a combination thereof contained therein, information regarding the dosages, indications for use, and/or recommended treatment regimen(s) for the compound(s) and/or pharmaceutical formulations contained therein. In some embodiments, the instructions can provide directions for administering the compounds, compositions, formulations, particles, and cells described herein or a combination thereof to a subject in need thereof.

### EXAMPLES

Further embodiments are illustrated in the following Examples which are given for illustrative purposes only and are not intended to limit the scope of the invention.

### Example 1

### Results

### TnsC Phylogeny Reveals the Diversity of Target Selectors in Th7-Like Transposons

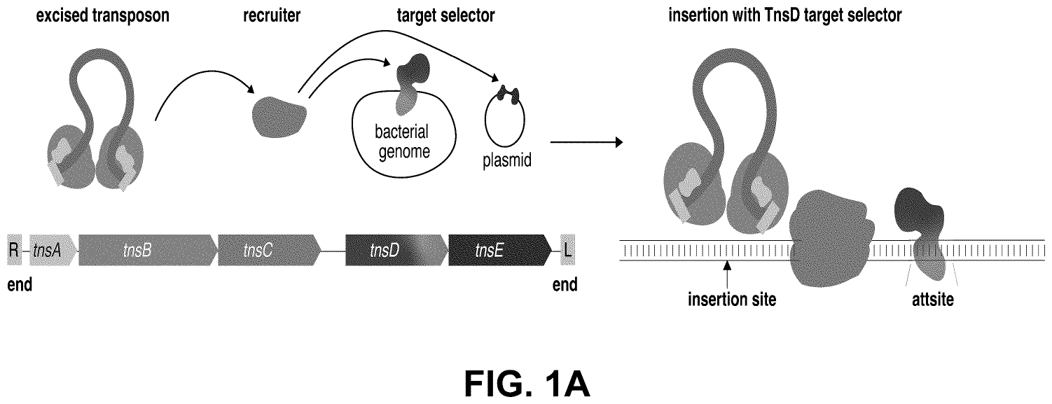

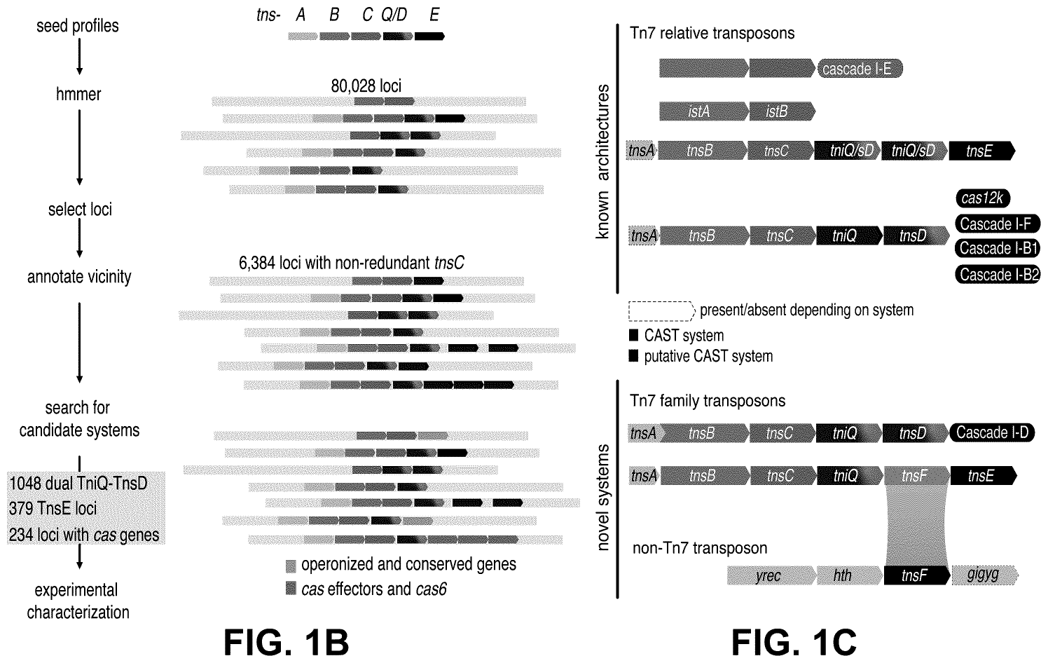

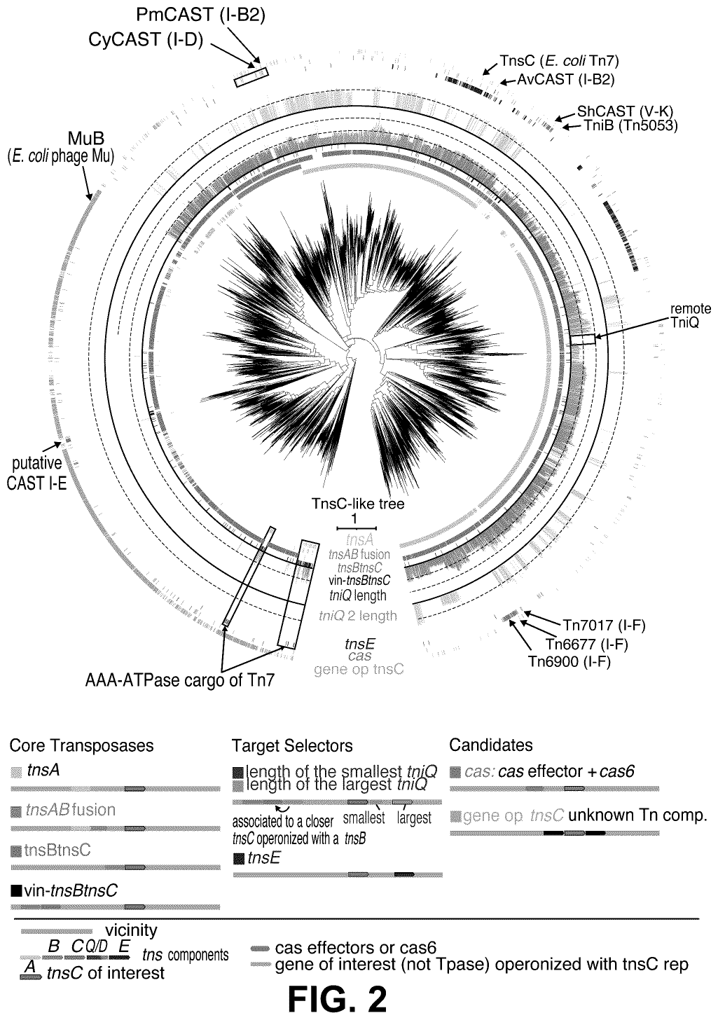

Known target selector proteins exhibit a wide range of diversity, but they all use TnsC to bridge target selection function with transposition. Applicant therefore used phylogenomic analysis of TnsC, which is more conserved, as a gateway to investigate the diversity of target selectors and search for new ones (FIG. 1B-1C). Applicant selected 80,028 Tn7-like loci identified in publicly available prokaryotic datasets from NCBI, JGI, and MG-RAST and analyzed their genomic neighborhoods to identify candidates associated with Tn7 components using a phylogenetic tree of a representative set of TnsC. Applicant selected 6,384 protein homologs of TnsC to build the tree. The tree included two main clades, one consisting of MuB, the homolog of tnsC from Mu phage-transposons (MuTn), and the other one of TnsC from Tn7-like transposons (FIG. 2). Applicant refers to BI-11050_All_Tn7_Systems (herein referred to as Table 8) and BI-11050_Tn7_Tsy_TnsF_Systems_BROD-5710 (herein referred to as Table 9) from U.S. Provisional Application No. 63/442,710, filed Feb. 1, 2023 to which priority is claimed herein. Reference is made herein to Faure, G., Saito, M., Benler, S., Peng, I., Wolf, Y., Strecker, J., Altae-Tran, H., Neumann, E., Li, D., Makarova, K., Macrae, R., Koonin, E., and Zhang, F., Modularity and diversity of target selectors in Tn7 transposons, Modular Cell, 83, 2122-2136.e1-e10, Jun. 15, 2023. Supplemental Information at https://doi.org/10.1016/j.molcel.2023.05.013, the entire contents of the which is fully incorporated herein by reference.

To explore the diversity of target selectors, the known target selectors (tniQ/tnsD, tnsE, and cas genes from CAST systems) were mapped on the tree. tniQ/tnsD is ubiquitous in the Tn7 branch and is represented either by a single gene (2,905 loci) or as tandem genes (1,048 loci). These tandems consist of either two tniQ genes, or tniQ and tnsD, or two thsD genes, which are collectively referred to as dual tniQ/tnsD. In contrast to the ubiquity of tniQ/tnsD, tnsE is more restricted and is present in transposons closely related to the canonical E. coli Tn7 and in the more distantly related group of Tn6022 transposons (379 loci total; FIG. 2). The CAST systems are spread around the tree and grouped according to their subtypes except for CAST I-B, which is found in two distinct clades (1 and 2), suggesting independent capture by two distinct transposons (Saito, M. et al. Dual modes of CRISPR-associated transposon homing. Cell 184, 2441-2453.e18 (2021)). Tn7017, a CAST I-F variant that uniquely harbors a dual tniQ/tnsD and uses TnsD for protein-mediated homing (Klompe, S. E. et al. Evolutionary and mechanistic diversity of Type I-F CRISPR-associated transposons. Mol. Cell 82, 616-628.e5 (2022)), belongs to a branch distant from other CAST I-F, which use a dedicated spacer for RNA-guided transposition (Petassi, M. T., Hsieh, S.-C. & Peters, J. E. Guide RNA categorization enables target site choice in Tn7-CRISPR-Cas transposons. Cell 183, 1757-1771.e18 (2020)). This branch consists of transposons encoding tns C and dual tniQ-tnsD, but mostly lacking Cascade I-F, suggesting multiple gains or losses of Cascade I-F.

### Identification of CAST I-D Subtype

To identify potential novel CAST systems, a search was performed for cas genes encoding CRISPR effector components (including Cascade subunits Cas6 and single protein Class 2 effectors) in the vicinity of tnsC and mapped the detected cas genes onto the tree (FIGS. 1B-1C and 2). Altogether, 234 groups of loci (nodes in the tree) harboring at least one of these cas genes were identified. Manual examination of tns C tree branches bearing cas genes showed that several of these genes are part of the cargo and are unlikely to be involved in transposition (Benler, S. et al. Cargo genes of Tn7-like transposons comprise an enormous diversity of defense systems, mobile genetic elements, and antibiotic resistance genes. MBio 12, e0293821 (2021)) or belong to already reported CAST systems. However, one group of loci was identified in the Tn7 clade in branches closely related to CAST I-B2 that encodes transposase components closely similar to those of I-B2 PmCAST, with ˜50% sequence identity between TnsABs and TnsCs, and ˜30% sequence identity between the dual TniQ-TnsDs (FIG. 8A). However, these loci contain Cascade I-D, rather than Cascade I-B, and thus comprise a novel CAST variety.

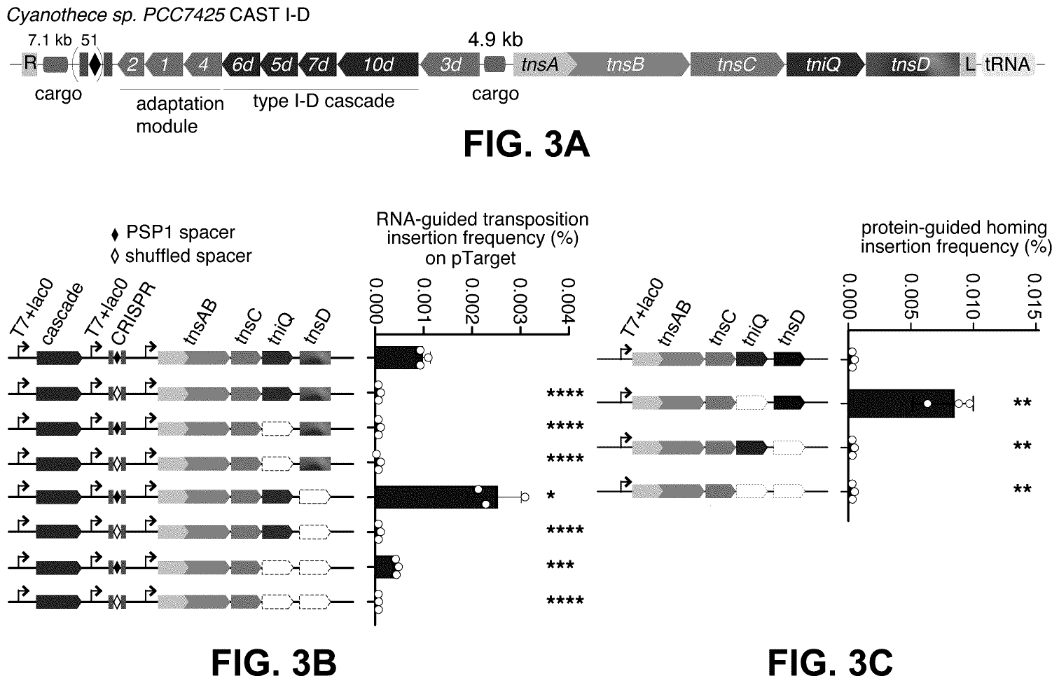

To experimentally characterize CAST I-D, Applicant chose a locus from the cyanobacterium Cyanothece sp. PCC 7425, CyCAST, that encodes a complete subtype I-D CRISPR-Cas system encompassing both the adaptation module (Cas1, Cas2 and Cas4), the Cas6 processing nuclease, and the Cascade complex, along with TniQ and TnsD (FIG. 3A). Unlike other known CASTs, the CRISPR-Cas system of CyCAST appears to be fully functional, that is, competent for both adaptation and interference based on the conservation of catalytic residues in the HD nuclease domain of Cas10d, suggesting that this is a recent acquisition of a CRISPR system not yet fully domesticated by Tn7 (FIG. 8B). Similar Cas10d proteins are also found encoded in loci where Tn7 components are absent in the vicinity (FIG. 8B). CyCAST Cas10d and orthologs are described in Table 5, below. Manual identification of the CyCAST boundaries indicated potential attachment sites in a tRNA gene, similar to the attachment site of CAST I-B2 systems (FIG. 8A).

TABLE 5
Table 5.

| Cas10d | Amino Acid Sequence |
|---|---|
| Cyanothece sp. | MKVFLKPLYSQLNPGIGHCPLGCEQVCQVQQQAPSLQPPPGYTC |
| PCC 7425 | PLSIHQANTCAKVVQGTADVIFNKAATGDGKSLAAYAPSLLNPN |
| (ACL44814.1) | VRIMGLYPTIELVEDQTRSQQGWHSMFRLDADERIDRLFGEELS |
| | RRVQEAETSNKFQELLLAIQQKPILLTNPDIFHLITHFQYRDPAYG |
| | NDLLPLALAEFPDLWVFDEFHIFGPHQEAAVLNSMTLIRRTQSD |
| | QRPRRFLFTSATPKPDFINQLQQSGLHTVEITGSYASEPTPGFRQV |
| | LQPVELEFVNLKDSDSLGWLAENANIIRDILQTESQGRGLVILNS |
| | VAQAGRAARLLAELLPNILVREISGRIDRQGRSETQLDLRNCPQP |
| | VLVIGTSAVDVGVDFKIHLLIFESSDSATVIQRLGRLGRHPGFSSY |
| | RAFVLLSGRAPWVMARLQERLQPDQLIERSELQEAIVEAFDAPK |
| | EFQEYRNRWGAIQAQGMLWKMGEGNSKVMQPVRDRMVADLQ |
| | KVYGSRLKSWLGDWKNLAKTDVGKETQQELLRFRGGSTLQAA |
| | VWDGDRFYTYDLLRLLPYTLVETTTRETFLEAATKAGLEKEAFP |
| | KNYIEIYLRVLEWVDDRFNISLHCNWNSSDLKCCELTLLNRLSL |
| | VGHLQSEVGRCFYSKKLLAFLVPLGMRQLHWEVSRHLKLSPVF |
| | GLHYLIDGSNKQGYACAFNQDALLLDALKSRLTKFCRLQPQSLI |
| | F (SEQ ID NO: 69) |
| Cyanobacteria | MATLLQTLLLQTLDLDPVLLAYIETVLPALEQEFGMLSAFDKAW |
| bacterium FACHB- | NGWFVQPEELDRMQQATQSSNNLGQSLLICILNSLLIVWNLLSFL |
| 471 | RAKLMLSDNEKRLLCLGFTFQGYNNGCLSKATALLPTSDITLGE |
| (MBD1866148.1) | TLNLDAFWMEWREYLSEINYLAQITPKIARKNTSSVKRNTYKISE |
| | CRLKQPLHQLLLFGTIAAQLSGPADIVVTPEGNCLQELMNELGIA |
| | RTLIYHRLRNCTGLLTNSIHNAVLQFTEKLDWQPILFFPDGIIYLA |
| | PSDSRIPAHIEFQKFVWEQVRDQLIDSISRGEAGFKRDGKGLKAA |
| | PQTLELFSPARLIRNLPSIMKAYVRNEKEPATPKRLARLSLDPTER |
| | EFLARGAGLRSDRLAEFLILVQREFFRNRPEFIAWILEKLEIQSDIT |
| | PEQTORNAGGVNLGWYYAAAHYVTKHSTPNSRNSIPNYFLVEL |
| | QDLAEQLATWAEQNHLLPTHPNLTREIFYDYVTQYLEIQGWDV |
| | QPTSFQRELAGYVNAKTKLAKQPICSLSSGEFLSEDQMDSVVLF |
| | KPQQYSNKNQLGGRQLKRGISKIWSLEMLLRQAFWSVPAGKLE |
| | DQQPIFLYLFPAFVHAPQITKAIRLLVKQLKQVNLWEICRFWQEH |
| | GMDANALRFYPWLRTDLDPNAEDKRDLPFLAVIYTTTKGKTTT |
| | DAWIKPAFLAIALPLLLGVKVVATASSVPFYNRGSEFQATVQVD |
| | ETASFWSLLSLPTTLHLEEHSQGQTKHLSDWLDRLLIAYTIHLDC |
| | RAKRPDPQWQAFPGTVQAIVTDVSAIFSLARSRFQSLSDDRVQQ |
| | YWEFAQIWATGDLDMEQQLELTQRLVQEYRTFYRVHVAKSSH |
| | AIVLPLSKVLEDILSTSNDLPIETLILQTAGRLHDALKRQEPYKRP |
| | LIMDKSLSIEIRVKNEMQAVHQFVTTCIKDLFLGQYKGDRALLQ |
| | EHRHRVKSGAEFAYRNL (SEQ ID NO: 70) |
| Oculatella sp. | MATLLQTLLLQTLPADLDPVLLAYIETVLPAVEQKFGMLPVVEG |
| FACHB-28 | SSNDRFVQLGKLDEVQRVFYSTNDSDQSLLIFILNGLLTVWNLLP |
| (WP 190646788.1) | FLSKKLALSDSEKRLLCLGFTFQGYRNLWQHSAKTSERTPVILLG |
| | ETLNLDAFWAGWQEYLPELTYIVPTIYQTAGERIAPTKQSPYKIP |
| | EPRLKLLLNQLLTFGKIAAHISDPADIVVTLRGNYLQGVMNELGI |
| | NRTLIYHRLRNCIGLLTNSIHNAVLQFTEKLDWQPILFFPEGIIYL |
| | APPNSGVPTHIEFQEFAWKQISDRLIDSMLRGEAGFKRDGKGLK |
| | AAPQTLELFSPAQLIRNLPSIIKAYVRNEKEPATPKRLVRLSLDST |
| | EQEFLAGGADLRSDRLAEFLILVQREFFRNRPEFIAWILEKLEIQS |
| | DITPEQTQRNAGGVNLGWYHAAAHYVAKHPTPNSNNPVPNHFL |
| | IELQDLAEQLATWAEQNQLLPTHPNPTREIFYDYVTQYLEIQSW |
| | DVQATSFQRELAGYVNAKTKLAKQPICSLSSGEFLSEDQVDSVV |
| | LFKPQQYSNKNSLGGRQLKRGISKIWSLEMLLRQAFWSVPAGKL |
| | EDQQPIFLYLFPAFVHAPQITKAIRLLVNQLKQVNLWEIRRFWQE |
| | HGMDANALRFYPWLRTDLDSNTEDKRDLPFLAVTYTTTKGKTT |
| | TDAWIEPAFLAIALPLLLGIKVVATASSVPSYNRGSEFQATAQVD |
| | GPASFWSLLGLPTTLHLEEHLQEQSKHLSDWIDRLLIAYTIHLDC |
| | RSKPPDSRWQSFPGTVHAIVTDVLAIFSLARSHFQNLSDDKVQQ |
| | YWEFAQIWAAGDLNMEQQLELTQRLVQEYRMFYRVHVAKSSY |
| | AIVLPLSKVLEDILSTSSDLPIETLMLQTAGRLHDALKRQEPYKRP |
| | LIMDQSLSIEIRVNNEIQAIHQFVTTCIKDLFLGQYKGDRALLQEH |
| | RHRVKSGAEFAYRLLAMQESESKTKQQDVEGAQL (SEQ ID NO: |
| | 71) |
| Kovacikia minuta | MTTLLQTLLLQTLPPDLDPVLLAYIKTVLPAVEEPGMISALNGLL |
| (WP_224344603.1) | TTWNLLPLLNSKFALSDDEKRLLCLGFTFQGYGHPCQSRTFELD |
| | TFWAEWREYLPEINYLAQITQQTVGKNSALARKTAYKIPERRLK |
| | LPLHQLLSLGTIAAQLTDPADIVTKPAGDRLREHLKALGIDRTLT |
| | YHRLRNCTGLLTNSIHNAVLHFTEKLDWQPLLFFAQGVVYLVPS |
| | DSELPDRSKVQAFLWEQISGSLATRMLNGKIGCKRAGQGLKVA |
| | PQALELIPPAQLIQNLPGVVAVTVKNETDPATLKRLARLDLRPTE |
| | REFLARGADLRSDLIAEFVIFLQKEFFGNNPEFITQVLDQLAIRLA |
| | ITPEQTQVRTGGFYWGWYHAAAYYVASNPHLESEDVTAKLQD |
| | LANQLVTWAEEKKLLPVRSSSTREGFDRYLTQHLELPGWDNPEP |
| | SFQQELATYIAAKTKAAKQPICSLSSGEFASEEQMDSVVLFKPQQ |
| | YSNKNPLGGRQLKRGISKIWSLEMLLRQAFWPIPAGKLEERQPIF |
| | LYLFPAFVHAPQVTKAIRLLVNQLKRVNLWEIRRFWQEQGMDS |
| | NALRFYPWLRDNPNPDENDKRDLPFIAVTYTTTNGKTTTDAWV |
| | EPAFLAIALPLLLGMKVVATASPVPLYNSSSEFSAKAQVDGPPSF |
| | WHLLGLPTSMHLEEEIQGQGKHLSDWLDRLLIAYSIHLDCQAKP |
| | PKTHWQAFPGTVQAISTNITAIFSLAHSHFQSLFDEKVQQYWQF |
| | AQIWATGDLEMEQQLELTQRLVQEYRTFYRVHVAKSTHAIVLP |
| | LSKVLEDILSTPSDLPFEDMLLQISGRLHDALRRQEPYERPLIMD |
| | QSLSFEIRVNNEIQAVHQFVKTCIKDLFLGQYKGDRALLQEHRN |
| | RIKSGAEFAYRLQAMQEPKSKTKKQEIEGAQR (SEQ ID NO: 72) |
| Myxacorys | MKPLLQTLLLQTLPSDIDPVLLAYVETVLPAVEQEFGLIPVSNDV |
| californica | SQTEQSLLVHILNSLLTAWNLFPFLSSKLALSDNEKRLLCLSLIFQ |
| WJT36-NPBG1 | NDNSKCQSRENNSPPTLGVNETLKQYRAFGDKLNLAAFWTEWR |
| (MBW4418978.1) | EYLTDIVYLTQSTQPHALSIKKTALKIPANRLELPLRPLLLFGAIA |
| | AQLTDPADIVIKPEGNVLKECLKRLGVQHQLTYHRVRACTGLLT |
| | EGIHNAIMRFVQPLDWQSILFFAQGIVYLVPSDSKLLDCSKLQTFI |
| | WEQISEQLAISMLRGKVGFKRDGKGMKAAPQTLELFSPAQFIRN |
| | LPTVINACVKNETEPATPKRLANLDLDPTDREFLACGADLRSDRI |
| | AEFILFLQKKFFGTNPEFIDRVLDQLGIQSAITPEQTQIFSSGVNRG |
| | WYHAAAYYVASNPHLDSDDVTIKLQEIANQLVDWAEEKELFPP |
| | QISPTREVFNQYLAQYLEFPRWNNSAPSFQHELATYVAAKTKAA |
| | KQPICSLSSSEFDSEDQLDSVVLFKPQQYSNKNPLGGWQLKRGIS |
| | KLWSLEMLLRQSFWSVSAGKLEERQPVFLYFFPSLVHSPQVAKA |
| | IRILVNQLKRVNLWKVCRFWQVHGMNANTLRFYSWLKSDPRP |
| | DDNGKRDLPFLAITYTTTAGKTTTQAWIEPAFLVLALSLVLGIKV |
| | VATASPVPLYNRSSEFPETVKIDGPASFWSLLGLPTSIRLEEKIQG |
| | RVKHLNDWLDRLLIAYSIHLDCQANPPDSRWQLFPGTVQAITTD |
| | VSAIFSLAHSHFQSLSDDRVQRYWHFAQVWAAGDSNMEQQLEL |
| | TQRLVQEYRAFYQVHVAKATHAIVLPLLKVLEEILSTPSDLPIET |
| | LILQTSGRLHDALKRQEPYKRPLIMDQSLSIEIRVNNEIQAIQRFV |
| | TTCINDLFLAQYKGDRALLQEHRNRIKSGAEFAYRLLAMQEPKP |
| | KTKKQEIEGAQI (SEQ ID NO: 73) |
| Trichocoleus sp. | MAIALPLVLGIKVTATTSSAPPYNRDSDSEFLTTVQVDGPASFW |
| FACHB-46 | NLLDLPTRIHLEEERKGQVKHLSDWLNRLLIAYSVHLDCRSKPP |
| (WP_199299221.1) | DPRWQAFPGTVHAITDDVLAIFSLAHASFPVLSDEIVQRYWKFA |
| | QIWTAGNPAMKQQLDITQRLVQEYRVFYQVHVAKSTYAILLPLS |
| | KVLEDILSTPSDLPIETLILQTSERLHDALRRQEPKRRPLVKDHSL |
| | SIEIRVNNEIQAVHQFVSTCVNDLFLGQYKGDRALLQEHRSRIKS |
| | GAEFAYRLLAMEETKPKTKKQEVEGAQL (SEQ ID NO: 74) |
| Synechococcus sp. | MTLLQTLLLRTLSEETDPILRAYLATVLPAMEQEFGLIPALGGSE |
| C9 | EAQYHLLLQRGSEFAMEKAKRYAEYSDQSLLVHVLNALLTAW |
| (WP_247216937.1) | NLLPFLSPELILTDAEKRLLCLGLTLHDYNKWCNGEQQQVPDAS |
| | EIPKILELCQTLGERLNFDAFWAEWREYLTEIGYLAQNTQGKVGI |
| | NPFKANWGTFKIPDTRRLDSPLRWLLAFGDVAVHLSDPSDIETK |
| | TAGNRLRDHLRHLGIHRKLTYHRLRDCTGILTSGIHNAVMHFAR |
| | ELDWQPLLFFAQGVVYLAPQDTGVPDRSELQEFLWEQVSKLLA |
| | SNMENGQVGFKRDGKGLKVAPQTLELFSPAQLIRNLPSVVKSYV |
| | ANAKSPATPKRLERLEPSEIEREFLSSSADLRADRTAEFIILAQKE |
| | FFADRPEFIAWMLEKLGVQDAITPEQTQFQSGGVNYGWYYVAA |
| | YYIARHSTLDLEQLDKKLQEIAEQLATWAEENKLLPEHYSPTRE |
| | VFYDYLTQYLEVQGWEQPESSFQRELVGYISAKTSKAKHPICSLS |
| | SGEFASEDQMDSVVLFKPQQYSNKNPLGGTQIKRGISKIWALEM |
| | LLRQIFWSVPAGKLEDQQPVFLYIFPAYVYSPQVAAAIRLMMNE |
| | LKRINFWDVRKYWLEHGMDAQALRSFSWLTNDDGDAGNSAPS |
| | KYSREDLPFMAMHYTTTRGKTVTEAWIEPAFLALALPLLLGVK |
| | VVATSSQIPLYDSDKDFKESVKIDGPANFWYALGFPTSLHLQDW |
| | IGGRVQNLADNLNRLMVAYCLHLDSRSNPPDACWRKLNDTDF |
| | ANKHSGLGGTNPPDERWRKLNDTIQQIVASVLNVFSLAKEGLRR |
| | SSSKDKRKPTKNEINRYWSYAHIWAEGDTEMETKLKITKRLANE |
| | YRQFYRVGIDKSSYAILLPFSKALEHILSVPNDWDDEELIVQGAG |
| | QLRAALERQEVYKRPIIQNKEIPFEQRRIQEFEAIQTFMTTCVIDV |
| | FGRMCKGDRALLQEHRNRLKSGVEFAYLQLALQDQQATS (SEQ |
| | ID NO: 75) |
| Kovacikia minuta | MTTLLQTLLLRTLPPDTDPILRAYIETVLPAMEREFGMIPALGGS |
| (WP_224346160.1) | EEAHYYLQKQRGNKFAAENARRYASRSDQSLLVHVLNALLTG |
| | WNLLPFLSPALAVSEDEKRLFCLGLTLHDYNKWCQGEEDSPHA |
| | SEVADILKLCNTLGAQLNFDAFWVDWREYLTEIAYLAQNTQGK |
| | VGTNLPKTNWGSFKLPDTRRLDSPLRWLLAFGDVAVHLSDPADI |
| | VTKTGGDRLREHLRFLGIRRRLVYHRLRNCTGILTNAIHNSVLRF |
| | VQEFDWHPILFFAQGIVYLAPLDEEIPDRLSIQEFLWERISQFLAE |
| | EMLGGQVGFKRDGKGLKVAPQTLELFSPAQLIRNLPGVIQSYVN |
| | NAKSPATPKRLEKLELSQTEREFLARGEDLRADRLAEFIFLAQKE |
| | FFGNRPEFIPWMLETLGIQSAITPEQTQLQSGGVNWGWYHAAAY |
| | YVASNATLDLNGLSEKLQDFAEHLATWAEENGFFPEHLSPTRDI |
| | FSNYLAQYLEVQGWDEQERSFQYELASYTVSKTKAARQPICSLS |
| | SGEFASEDQMDSVVLFKPQQYSNKNALGGRQIKRGISKIWSLEM |
| | LLRQALWSAPAGKLEEQQPVFLYIFPAYVYSPQVASAIRLLVND |
| | LKRVNLWEIRKHWLQNGMDVEALRSLPWLNTSGD (SEQ ID NO: |
| | 76) |
| Trichocoleus sp. | MNSLLIVWSLIPLLNEELALSKDEKCLLCVWFTLQGYNSCYSSR |
| FACHB-46 | VTAPLLTPDINLGEVLNLDMFWPQWREYLPEIDYLTQITLQNSD |
| (WP_206755453.1) | NNTSAKRNGFRVPDQRLRLTLHPLLSFSTIAAQLTSPVDIVTTSA |
| | GDRLPALLKELGIDRTLIYHRLRNCTGLLTNSIHNAVLNFTEKLN |
| | WQPILCFTEGVVYLAPRGFNLLNRMDLQQFIWEQISDQLVTNML |
| | VGEIGFKRDGKGLKAAPQILELLSPAQLIQGLPSIVEATIKNEIDP |
| | ATPKRLMNLSITTLERKFLSQGADLRSDRIAEFIIFLQKQFFRSNS |
| | EFIAWILEQLGIQSAITWEQAQIHTGGVNLGWYHAAAYYVANSP |
| | HLDLRSVTRKLQEIASRLTIWAEEKKLFPVLTSATREAFDRYLVQ |
| | YLELPGWDSPESSFEHELATYMAAKTKAARQPICSLSSGEFVSEA |
| | QMASVVPFKPQQYSNKNPLGGRQLKRGISKIWSLEMLLRQAFW |
| | SFSPGKLEERQPVFLYFSPTFAHAPPVTKVIHILAKQLERVNLWKI |
| | RRFWQEHGMDANALRFYPWLKNELNSNEESQHNLTFLAMTYT |
| | TTKGKNNDRCLD (SEQ ID NO: 77) |
| Leptolyngbya sp. | MTTLLQTLLIRTLPPDTDLILKEFIDTVLPAMETQFGHMTALGGS |
| SIO4C1 | QAVHEHRLRKMNDAYATEKAQRWASSPDQSLLVHVTNALLLA |
| (NEP20205.1) | WTLTPFLSEPLSDNEKRLICLGLTLHDYNKYCQGEEEDVPKAHE |
| | VNEILTLCEEMGERLNFSAFWSDWRQYLSEIGFLAQNTQGKIGT |
| | NLIGTNWPKFQLRERRLKNPLRPLLRFGDVAVHMANPAELAMP |
| | ATGRTRPRGKALKDCLEDLGIKGELTYHRLRQPTGILSNRVHNA |
| | VLHFTAALDWQPILYFAQGVVYLSPLSPTAPKRETLKKALWQSI |
| | SQFLESQMMNGEIGFKRDGKGVKVAPQTRELFESTQIIRELPGVI |
| | AANVRNEKDPATPKRLASIGMDATERKALMAVADLRCDLIAER |
| | (SEQ ID NO: 78) |
| Leptolyngbyaceae | MATLLQMLLISTLSSDTDPTLRQFIDTVLPSMEQEFSSITALGGSY |
| cyanobacterium | EVHLSRLKHKETAKRWSEKADQSLLVHVLNGLLTAWNLIPFLD |
| CCMR0081 | DNPLNDDEKRLLCLGLTLHDYNKYCQGEEKDSPKAYEVDSILA |
| (NEZ54669.1) | LCQEMGKRLNFGSFWPEWKSYLSEIGFLAQNTQRKHGSNLVLA |
| | NWPNFKLRRHRLLNPLRLLLTFGDVAVHMTNPAEVITTTRGDRL |
| | QDCLDDLCIPRKLVYHRLRDVTGILSNGIHNAVLAFTKQEKWQP |
| | ILYFAQGAVYLAPQGTKVPESEKLKSVLWNAIEKILSGAMLGGN |
| | IGFKRDGKGVKVSPQTLELFSPEQLIRGLPEVITAKVRNDKAPAT |
| | PKRLASLELSESELERLAAGSDIRSDQLAELIGLVQKEFFSKTPEF |
| | TPWVLEQLKIQDEITTEQTQVQKGGVNYGWYRAAAEYVLKHQ |
| | TLDTEQFAEMLGQFSTALADWAIEKQLLPETVNSTKDAFLSYLD |
| | QYLEISGWDSIGGSFQQELERYTIAKTKASKQPICSLSSGEFISED |
| | QMDSVVLFKPQQYSNKNPLGGGQIKRGISKIWSLEMLLRQSKW |
| | AVPPGKMEDQNPIFLYMYPAYVYSPQVAQAVRVFAKDLRQINF |
| | WELHKFWINHNLDIHALHQFPWLSEPDPEEWKLKNTQIYGSTGP |
| | KQDLPFLGISYTTTRGKTHTDAWAEPAFLSLVIAKFLGIKVVATA |
| | SQTPLYRSDSDFRESIKLDGLANFWSVLNLPDAIHLEEHHQGRIQ |
| | RIEDFMQRLLIAYSVHLENQADKPDPRWRALPDTVRQLMSNVL |
| | NLFSLAESGCRKRKRDPTPDDAQRYWKYAQLWSQGDAIMEEQI |
| | SLVKRLAQEYRQFYKVRSTASSHAILLPLTKALDTILSVPPDLPEE |
| | DLILEGAGQLKDALERQEVYTRPLLLDKSKDIALRKALELQAIQ |
| | QFMTTCVEQLFKEQYQKDIALLQENRNRIKSGVEFAYLMLNLED |
| | VQQEAESETETESAA (SEQ ID NO: 79) |
| Leptothoe | MATLLQTLLINTLAADTDPIFRQFIDTVLPAMEQEFGSITALGGS |
| spongobia | YEVHLDRLTRKKHPYAEETAQRWANSADQSLLVHVLNGLLTA |
| (WP_215607749.1) | WNLIPFLPDNDLTDTEKRLLCLGLTLHDYNKYCQGEEDDPPKAY |
| | EVDSILALCHEMGQKLNFESFWPEWKDYVSEIGFLAQNTQGKH |
| | GTNLAPRNWPDFKLRDRRLLKPLRPLLAFGDVAVHMTNPAEVE |
| | TTTRKDRLRDHLDSLGISGKLVYHRLRDVTGILSNGIHNAVLAFT |
| | AQEDWQPILYFAQGAVYLAPKKTEVPEPDHLKHALWNSIEELLS |
| | GAMLGGDIGFKRDGKGVKVSPQTLELFSPEQLIRGLPEVITAKVR |
| | NEKSPATPKRLDSLELSETERARLGTVADIRSDRLAELIGLVQKE |
| | FFSKTPEFTPWVLEQLKIQDEITPEQTQVQKGGVNYGWYRAAAE |
| | YVLKHQTLDTEQFSEILEEFVKALADWATEKQLLPETVNPTKEA |
| | FLSYLDQYLEISGWDSVGGSFQQELERYTIAKTKGSKQPICSLSS |
| | GEFISEDQMDSVVLFKPQQYSNKNPLGGGQIKRGISKIWSLEMLL |
| | RQSKWAVSSGKMEDQQPIFLYMYPAYVYSPQVAKAVRVLVND |
| | LKRVNFWELHKFLIEHDLDINALKQYPWLSETEPEEGRENDEKY |
| | AGQDLPFLGISYTTTRGKTHTDAWAEPAFLSLVIAKFLGIKVVAT |
| | ASQTPLYRSDNDFRESIKLDGLANFWSVLNLPDAIHLEEHHQGHI |
| | QRIEDFMQRLLIAYSVHLENQADKPDPRWRALPDTARQLMSNV |
| | LNLFSLAESGCRKRKRDPTPDDAQRYWEYAQLWSQGDAIMEEQ |
| | ISLVKRLAQEYRQFYKVRSTASSHAILLPLTKALDTILSVPSDLPE |
| | EDLILEGSGQLKDALERQEVYTRPLLLDKSKDIALRKAQELQAIQ |
| | QFMATCVEQLFKGQYQKDIALLQENRNRIKSGVEFAYLILNLED |
| | AQQETESETETESAA (SEQ ID NO: 80) |
| Cyanobacteria | MTLLQSLLTTTLEVDPDPILQAYIDTIVPAMEKEFGSIPALGGSEA |
| bacterium FACHB- | VHFYRLQKQGNTYADSTAKRWAEKADQSLLVHVLNGLLTAWN |
| 63 | LFPFLNQDKQLSEIEKRLLCLGLTLHDYNKYCQGEEDDAPKAHE |
| (MBD1847458.1) | VSEILALCEKLGQLLNFDAFWSDWRLYLADISFLAQNTQYKVG |
| | ANPVPSNWQTFKLEDSRRLKNPLRPLLAFGDIAVHINDPADVITT |
| | TRGNRLREHLRSLQIDRKLMYHRLRDCTGLLSGGIHNAVLYFTE |
| | PLDWQPLLFFGQGVIYLAPQNAEVPDRNDVQAVLWEQISQILGS |
| | KMLGGEIGFKRDGKGLKVAPQTLELFSPAELIRNLPEVVRTKVG |
| | NEKNPATPKRLESLDLGGDELEHLSKGADLRSDRLAEFIFLAQK |
| | EFFGNCPEFAPWILQRLGIQNRITPEHTQVQSGGVNYGWYYAAA |
| | CYVAAHASMRPEELLQTLEEVANDLADWAEENNLLPEHHSPTR |
| | DVFLNYLNQYLELSEWDSSVNQFAIEFNNYVAAKTKAAKQPICS |
| | LSSGEFISEDQVDSVVLFKPQQYSNKNPLGGRQIKRGISKIWSLE |
| | MLLRQALWAVPAGKLEEQQPVFLYLFPAYVYSPQVAKAVRVLI |
| | NELKRVNFWDLRKFWFEHDMNVQALRHYQWLQNEDEPGRFSS |
| | KDYGRRDRRDLPFMAITYTTTREKTTTDAWVEPAFLALALPFLL |
| | GIKVVASASPAPLYQSDREFRESVKLDGAANFWNLLGMPSSAHL |
| | EEFHQGRILRLEDWLERLLVAYSIHLDCEGEPPKPRWQALPGTV |
| | REIMTDVLNVFSLADSAFRDAKRDPKADEVLRYWSFAQIWSKG |
| | DVRMEAQLQLTQKLVKEYRRFYQVNTGESSHAILLPLSKALEIIL |
| | TVPNDLPIEDLIFQGSGQLHDAIARQKPYSRPLIMDKSVAYETRL |
| | ANELQAIHQFMTTCVKELFGEQYKGDRALLQENRNRIKSGAEFA |
| | YRWIALQEKQTKPSV (SEQ ID NO: 81) |
| Halomicronema | MTLLQDLLTTTLQEEPDPVIQRFVETVVPAMEQEFALVPALGGS |
| hongdechloris | DAVHRYRLRDDPFCEEKVQRWNQSADQSLLVHVINAILTAWNL |
| (WP_080810414.1) | QTFLDEDKQLTEEEKKLLCLGLTLHDYNKYCQGEEEDAPKTHE |
| | VSEILGLCHKLGHKLNFTDFWQDWENYLGDIGFLAQNTQYKTG |
| | TNPRLEVWNPKITDQRRLKNPLRPLLAFGDIAVHMNDPADIVTP |
| | KEGNQSRSRGHALREHLETLQIERKLVYHRLRDCTGLLTTGIHN |
| | AVLHFTEDLDWKPILFFAQGVVYLAPLDSETPDRETIQAVLWEQ |
| | IQQLLANKMLSGDIGFKRDGKGLKVAPQTLEVFKPAQLIRGLPD |
| | VIIAKVGNAKNPATPKRLASLELSDTECQKLEPAADLRSDRLAEL |
| | IFLAQKEFFGACPDFVPWVLKYLGIEQGISPEQTQVQSGGVNYG |
| | WYRAAAYYIAVTQKNTLDNEELEKILENLAYSLADWAEENDLL |
| | PEYKSPTQDVFHRYLNQNLEVSGWEPCLTSFDDELSAYTAAKTK |
| | ASKQPICSLSSGEFASEDQMDSVVLFKPQQYSNKNPLGGRHIKR |
| | GISKIWSLEMLIRQAMWAVPAGKLEDQRPVFLYIFPAYVYSPQT |
| | AKVVRVLMDELKDRINFWDIRKFWQENNMDIQALRSYSWLEEE |
| | SEAGRFGNPNYGRGDRRDLPFVAITYTTTRGKTVTDAWIEPAFL |
| | AMALPMLLGVKVVASTSPAPLYSSDSEFRESVKLDGPAGFWNS |
| | LGLPNSLHLEEWLQNRVQRLDELLNRLMIAYALHLDCEGDPPDP |
| | RWRAFANTVRDMMTDVLNIFSLAASHFRELKREPYPDEVGRYW |
| | RYAQIWTEGNTNMQKKLKITKQLVTEYRKFYRVNLSESSHAILL |
| | PLSKALELILSVPEDWDDEELILQGSGQLQDALDRQKVYRPILSD |
| | KSLPYQERKVQELEAIQAFVTTCVKDLFGEMCKGDRALLQENR |
| | NRIKSGVEFAYRWLTLQESQAETKNQKTEGEK (SEQ ID NO: 82) |
| Nodosilinea sp. | MTLLQDLLTITLREEPDPVLQKFVETVVPAMEREFALIPALGGSE |
| LEGE 07298 | AVHRHRLRDDLYGEEKAQRWSQSADQSLLVHVINAILTAWNLQ |
| (WP_194024837.1) | PFLDEDKQLTEEEQHLLCLGLTLHDYNKYCQGEEEDSPKAHKV |
| | EEILRLCRELGEKLNFSAFWPEWQDYLSDIGFLAQNTQYKAGTN |
| | LVSINWPTFKIDSRRLRNPLRPLLAFGDIAVHMGNPSDILTTTRG |
| | DRLREHLDALQIDRKLVYHRLRDCTGLLSSGIHNAVLHFTKELD |
| | WQPILFFAQGVVYLAPQSSETPDRDTVQGVLWEQIQSLLASKML |
| | TGEIGFKRDGKGLKVAPQTLELFSPVQLIRGLPDVITAKVGNVK |
| | NPATPKRLESLGLSEAERESLEPAADLRSDRLAELIFLAQKEFFG |
| | DIPEFAPWVLEYLGIDQSITPEQTQVQAGGVNYGWYYAAAHYIA |
| | VVRDNTLDKDQSQEVLVAFSNALADWAEEHNFLDSHKSPTQDI |
| | FLDYLSQNLEITGWHQTLTPFGDELAGYVAAKTKAARQPICSLS |
| | SGQFASEDQMDSVVLFKPQQYSNKNSLGGRQIKRGISKIWSLEM |
| | LIRQAMWAAPAGKLEDQRPVFIYVLPAYVYSPQTAKAIRVLMD |
| | ELKDRINFWDIRKFWQEHEMDIQALRSYPWLKEKSEEGRFADSN |
| | YGRGEKRDLPFVAITYTTTRGKTVTDAWIEPAFLAIALPLLLGVK |
| | VVASTSPAPLYSSDSEFRESVKLDGPAGFWNSLGLPNSLHLEEW |
| | MQGRVQRLDEILNRLLIAYALHLDCEGDPPDPRWRAFANTVRDI |
| | MTDVLNIFSLAASHFRGLKREPYAEEVKRYWSYAQIWSKGNID |
| | MQEKLKITNQLVTEYRQFYKVHLSESSHAILLPLSKALEEILSVPE |
| | DWDDEELILQGAGQLQAALDRQEIYKRPILSDKSIPFLDRKIQEL |
| | EAIEAFMTTCVKDLFGEMCKGDRALLQENRNRIKSGAEFAYRW |
| | LTLQESQSQANTQQPEGEE (SEQ ID NO: 83) |

CyCAST was expressed heterologously in E. coli and tested for activity. Using previously established assays (Strecker, J. et al. RNA-guided DNA insertion with CRISPR-associated transposases. Science 365, 48-53 (2019); Klompe, S. E., Vo, P. L. H., Halpin-Healy, T. S. & Sternberg, S. H. Transposon-encoded CRISPR-Cas systems direct RNA-guided DNA integration. Nature 571, 219-225 (2019); Saito, M. et al. Dual modes of CRISPR-associated transposon homing. Cell 184, 2441-2453.e18 (2021)), Applicant determined that CyCAST exhibits a GTT protospacer adjacent motif (PAM) preference, as observed in subtype I-D CRISPR-Cas systems (Kieper, S. N. et al. Cas4 facilitates PAM-compatible spacer selection during CRISPR adaptation. Cell Rep. 22, 3377-3384 (2018)) (FIG. 8C). Applicant detected mainly unidirectional left end (LE)-cargo-right end (RE) insertions within a 70-80-bp window downstream of the protospacer on a target plasmid (pTarget) (FIG. 8D).

To clarify the roles of TniQ and TnsD in RNA-guided insertion, Applicant deleted either TniQ, TnsD, or both and checked for activity. The full CyCAST system with both TniQ and TnsD showed RNA-guided transposition at an insertion frequency of 0.001% (FIG. 3B). Removing TnsD boosted RNA-guided transposition about 2.5-fold, to 0.0025%, suggesting that TnsD partially inhibits this activity. Elimination of TniQ abolished RNA-guided insertion activity, but perhaps unexpectedly, in the absence of both TniQ and TnsD, a low level of RNA-guided transposition was detected (0.0004%). Thus, CAST I-D retains some basal RNA-guided transposition activity in the absence of a protein target selector, in contrast to the previously characterized CAST systems, which may indicate TnsC can recognize Cascade at the insertion site (FIG. 8E).

Applicant also tested CRISPR-independent transposition (homing) of CyCAST. To this end, Applicant cloned into the pTarget plasmid a tRNA leucine gene from Cyanothece sp. PCC 7425 to serve as a homing site. Homing transposition was observed when TnsAB, TnsC, and TnsD were expressed in the absence of TniQ (0.008%); the presence of TniQ drastically diminished this activity, but transposition was still detectable (0.0006%) (FIG. 3C). Homing transposition occurs around 30-33 nt downstream of the end of the tRNA homing site, resembling the insertion site of CyCAST in the genome of Cyanothece sp. PCC 7425 (FIG. 8F). Thus, like some other CASTs, such as AvCAST, a type I-B1 system, and Tn7017, CyCAST exhibits dual modes of transposition that rely on different target selectors, namely, the small TniQ protein for RNA-guided transposition and the larger TnsD for protein-mediated homing (Saito, M. et al. Dual modes of CRISPR-associated transposon homing. Cell 184, 2441-2453.e18 (2021); Klompe, S. E. et al. Evolutionary and mechanistic diversity of Type I-F CRISPR-associated transposons. Mol. Cell 82, 616-628.e5 (2022)).

Comparison of TniQ and TnsD reveals a modular architecture of target selectors

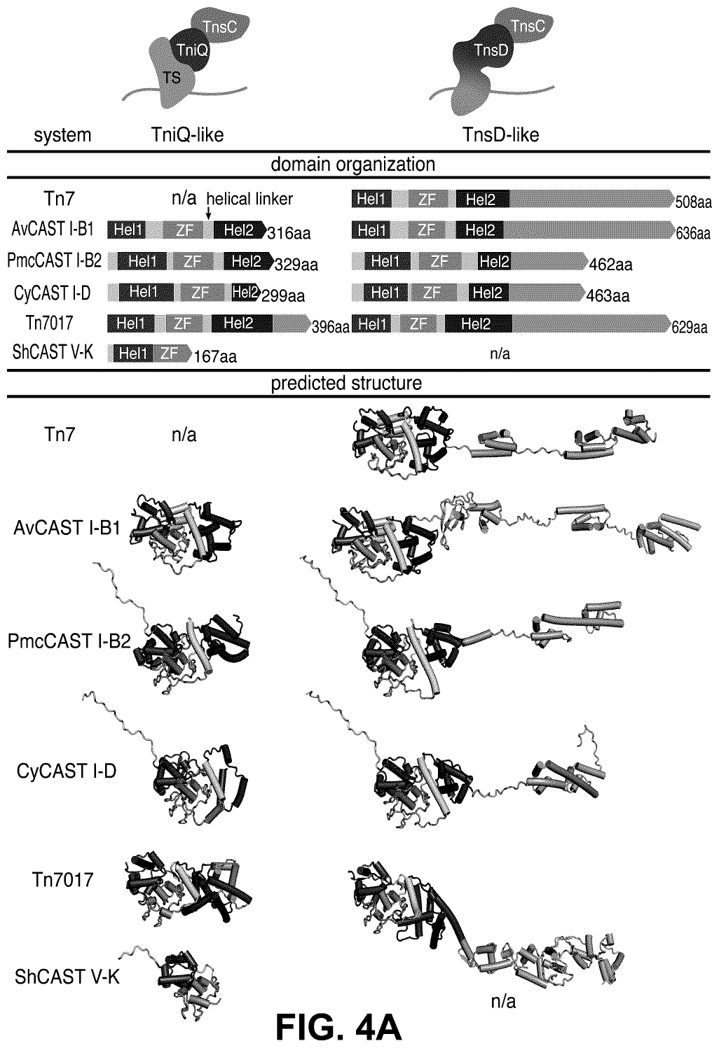

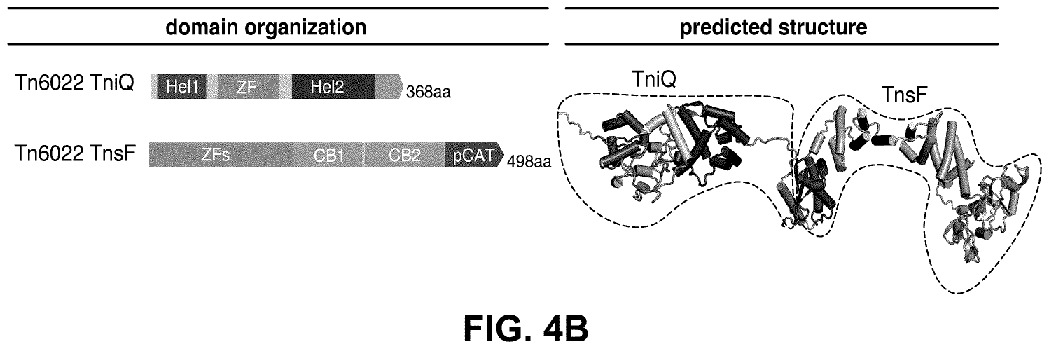

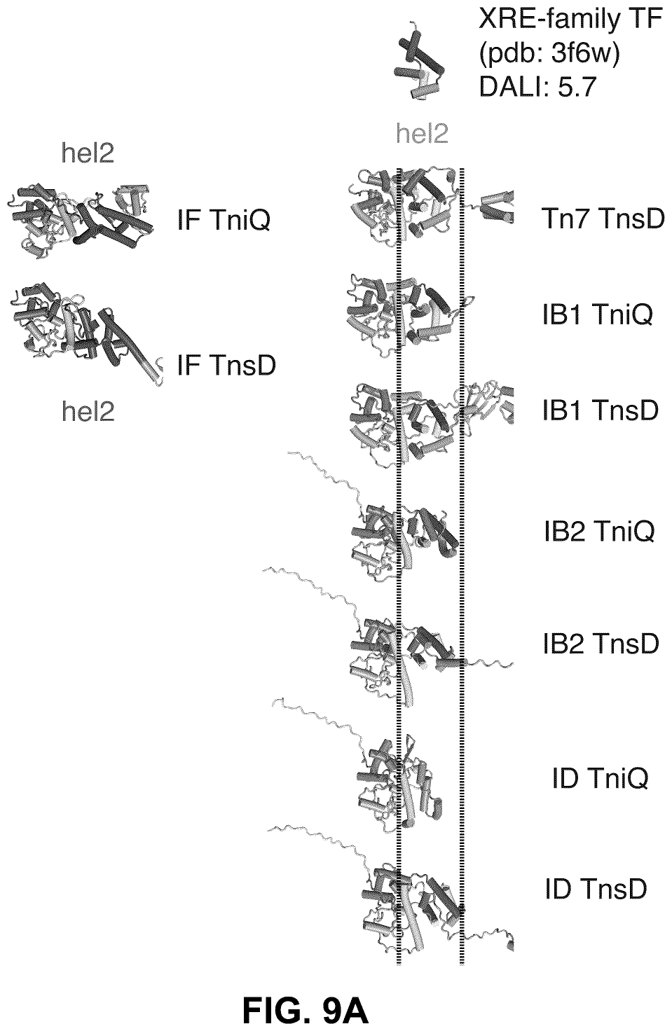

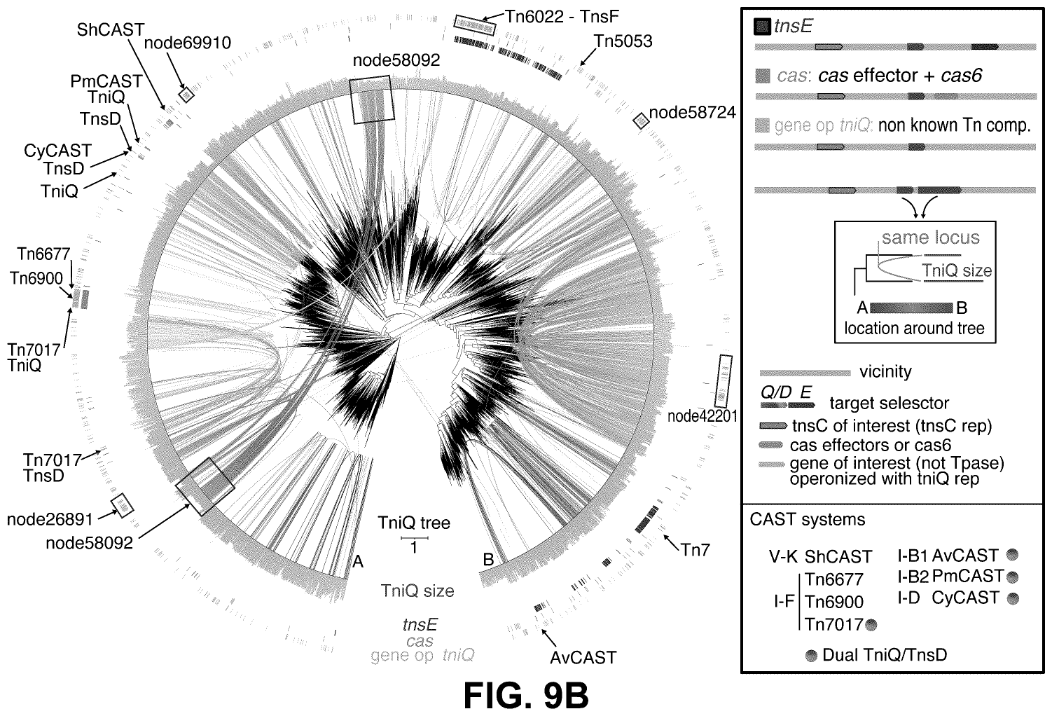

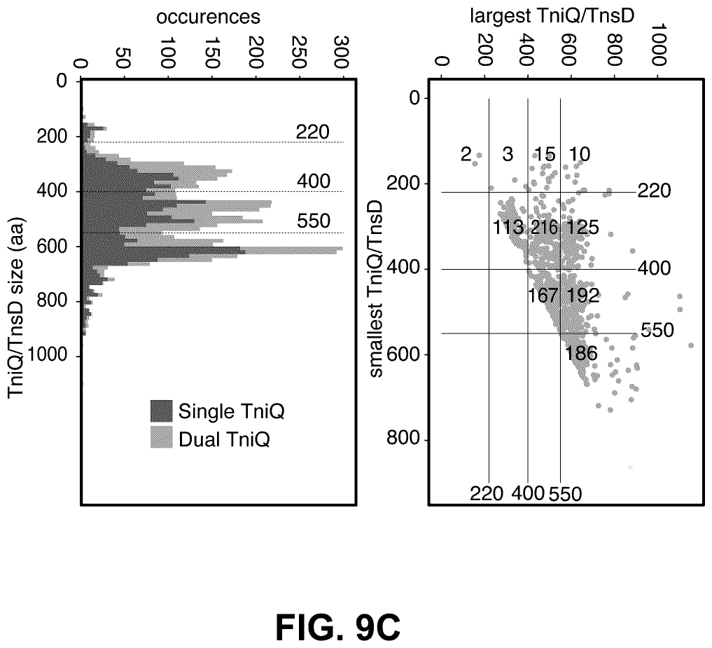

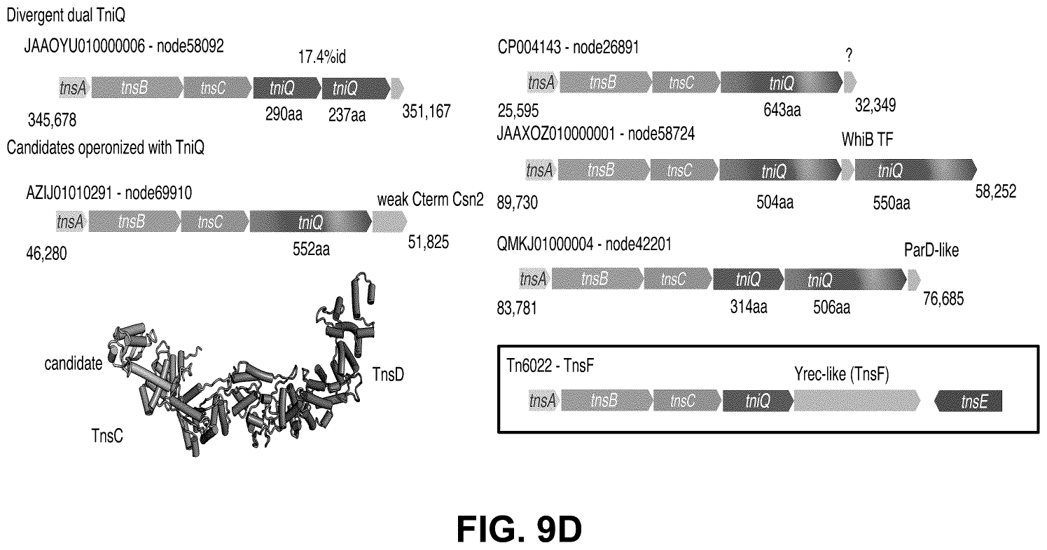

The presence of dual tniQ-tnsD genes in a variety of Tn7 loci motivated Applicant to examine these proteins in greater detail to gain additional insight into their roles in target selection. Mechanistic studies of CAST systems have shown that TniQ, which is smaller than TnsD, partners with Cascade to mediate RNA-guided transposition, whereas TnsD mediates protein-guided transposition (similar to its role in E. coli Tn7) (Saito, M. et al. Dual modes of CRISPR-associated transposon homing. Cel/184, 2441-2453.e18 (2021)). Applicant therefore first focused on TniQ and TnsD from CASTs and non-CAST Tn7-like transposons to determine how they function in these two capacities. Applicant employed structural prediction using AlphaFold2 (Evans, R. et al. Protein complex prediction with AlphaFold-Multimer. bioRxiv (2021) doi: 10.1101/2021.10.04.463034; Jumper, J. et al. Highly accurate protein structure prediction with AlphaFold. Nature 596, 583-589 (2021); Mirdita, M. et al. ColabFold: making protein folding accessible to all. Nat. Methods 19, 679-682 (2022)) to compare the domain organizations of TniQ and TnsD variants encoded by E. coli Tn7, CAST I-B, CAST I-D, and Tn7017 (the dual TniQ-TnsD CAST I-F) (FIGS. 4A and 9A). From the structural models, Applicant identified a common core structure shared by CAST TniQ and TnsD of about 300 amino acids (aa) that consists of a helical domain (HelD, a zinc finger (ZF), a connector helix, and another helical domain (Hel2). Even within this core region, however, there are notable differences among the TniQ and TnsD proteins from different transposons. For example, in some of these proteins, the Hel2 domain folds into an HTH domain that is similar to an XRE family transcription factor (TF), whereas in others, this domain has a unique fold (FIG. 9A). TniQ from Tn6677 CAST I-F interacts with Cas6 as well as Cas7 and the guide RNA through a loop in the Hel2 domain (Halpin-Healy, T. S., Klompe, S. E., Sternberg, S. H. & Fernández, I. S. Structural basis of DNA targeting by a transposon-encoded CRISPR-Cas system. Nature 577, 271-274 (2020)), suggesting that Hel2 provides a bridge to Cascade and that the structural diversity of Hel2 translates into compatibility with distinct Cascades. However, TniQ from CAST V-K, which associates with the Cas 12k effector rather than Cascade, contains only the Hell and ZF domains (Park, J.-U. et al. Structural basis for target site selection in RNA-guided DNA transposition systems. Science 373, 768-774 (2021); Querques, I., Schmitz, M., Oberli, S., Chanez, C. & Jinek, M. Target site selection and remodelling by type V CRISPR-transposon systems. Nature 599, 497-502 (2021)), indicating a different mode of interaction between the target selector components and highlighting the flexibility of TniQ as an adaptor between target selection and transposase machineries.

The CAST TniQ proteins consist largely of the core region, whereas the CAST TnsD and Tn7 TnsD proteins have a long C-terminal extension with broad diversity in terms of length, sequence, and structure (FIG. 4A). However, some of these regions share similarities that might reflect overlapping functions. For example, the TnsD proteins in Tn7 and CAST I-B, both of which home to the same site (glmS gene) (Waddell, C. S. & Craig, N. L. Tn7 transposition: two transposition pathways directed by five Tn7-encoded genes. Genes Dev. 2, 137-149 (1988); Saito, M. et al. Dual modes of CRISPR-associated transposon homing. Cell 184, 2441-2453.e18 (2021); Kuduvalli, P. N., Rao, J. E. & Craig, N. L. Target DNA structure plays a critical role in Tn7 transposition. EMBO J. 20, 924-932 (2001)), have common C-terminal domain architectures (one HTH domain following by a long linker and 2 HTH domains staggered by a short linker), which may indicate the C-terminal region is involved in the recognition of the attachment site. Applicant also detected similar structures and domain architectures (a long linker followed by a tandem of HTH domains back-to-back) between the C-terminal regions of TnsDs from CAST I-B2 and CAST I-D, suggesting they also recognize similar attachment sites.

Applicant's structural analysis of the models suggests that the conserved core region of TniQ and TnsD mediates the recruitment of the transposon via direct interaction with TnsC or via local conformational changes in the DNA near the target site, consistent with the observations of CAST V-K (Querques, I., Schmitz, M., Oberli, S., Chanez, C. & Jinek, M. Target site selection and remodelling by type V CRISPR-transposon systems. Nature 599, 497-502 (2021); Schmitz, M., Querques, I., Oberli, S., Chanez, C. & Jinek, M. Structural basis for RNA-mediated assembly of type V CRISPR-associated transposons. bioRxiv (2022) doi: 10.1101/2022.06.17.496590). By contrast, the C-terminal region of TnsD likely recognizes the attachment site during protein-guided transposition (FIG. 4A). The diversity of the interactions between TniQ and Cas effectors raises the possibility that the TniQ core acquired the ability to bind other target selector proteins as well. Furthermore, these findings suggest that the presence of a small TniQ lacking a large C-terminal extension is a general hallmark of Tn7-like transposons that employ additional target selectors.

### Identification of Novel Target Selectors

The analysis of TniQ and TnsD was expanded beyond the CAST systems to systematically analyze their diversity and identify potential novel partners of TniQ. To this end, Applicant extracted all TniQ and TnsD proteins encoded in the vicinity of the tnsC homologs in the Tn7 clade (4,916 altogether). Applicant found 2,905 loci encoding a single TniQ or TnsD and 1,048 loci encoding two distinct TniQ or TnsD proteins (FIGS. 1B and 9B). TniQ and TnsD protein size distribution comprises four sharply defined bins (210aa, 350aa, 450aa, and 610aa), suggesting selection driven by particular size constraints (FIG. 9C). However, TniQ and TnsD seem to represent two ends of a continuum, which is spanned by proteins containing extensions of variable length (FIG. 9C), making it difficult to predict, for some of these proteins, whether they bind directly to attachment sites or require a target selector partner. Therefore, some proteins are referred to as TniQ/TnsD, to emphasize that it remains unclear based on their size whether they interact directly with the target site.

The C-terminal target selector region of TnsD is highly diverse, and these proteins might have evolved by independent fusion of the TniQ core with other proteins containing DNA binding domains, such as HTH-containing transcription factors. However, the opposite directionality of evolution, namely truncation of TnsD upon recruitment of other target selectors, such as CRISPR effectors, cannot be ruled out either. The evolution of target selectors might have taken these alternative routes in different groups of Tn7-like transposons.

In dual TniQ-TnsD loci, one of these proteins is usually larger than 400aa, suggesting direct target selection, whereas the second one is substantially smaller (FIG. 9C). Such an architecture could provide transposons with multiple options for target selection: direct, via TnsD, or indirect, via TniQ interacting with an external target selector. Analysis of the phylogenetic tree of TniQ/TnsD built using the multiple alignment of the core regions indicates that dual TniQ-TnsD is polyphyletic (i.e., it emerged independently on multiple occasions via duplication of a single tniQ/tnsD (FIG. 9B), and monophyly of dual TniQ-TnsD (i.e., evolution from a single, ancestral TniQ/TnsD) was compellingly ruled out (p-value=2.4e-236). Dual TniQ-TnsD have likely arisen from in situ duplication of a single tniQ/tnsD (FIG. 9B), followed by neofunctionalization, and thus the ancestral Tn7-like transposons likely encoded a single member of this family. Such duplication might ensure compatibility with the other transposase components, while opening the possibility of evolving different modes of target selection. Consistent with the independent duplication scenario, only a few examples of dual TniQ-TnsD loci containing distantly related proteins were identified, as would be expected under an alternative scenario including exchange of tniQ/tnsD genes between different transposons (FIGS. 9B and 9D).

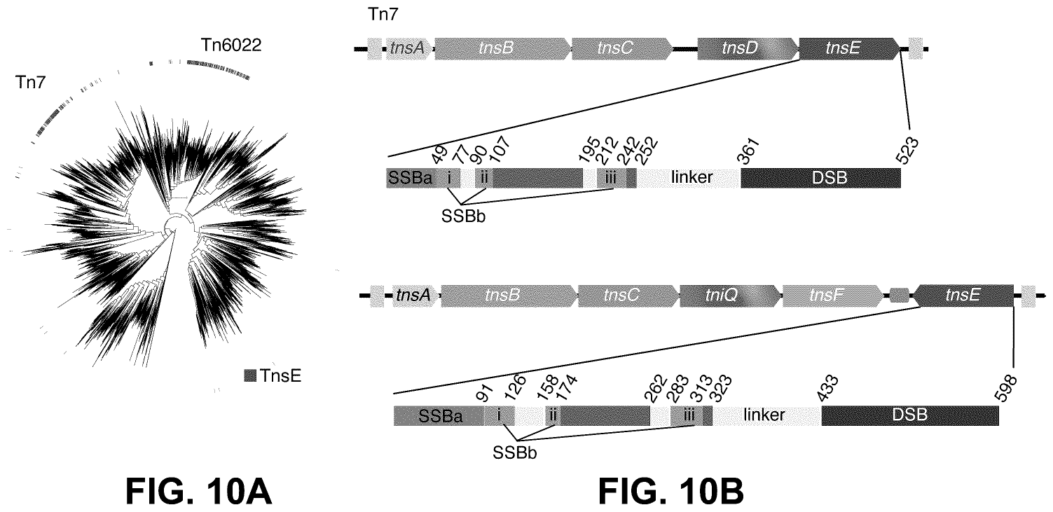

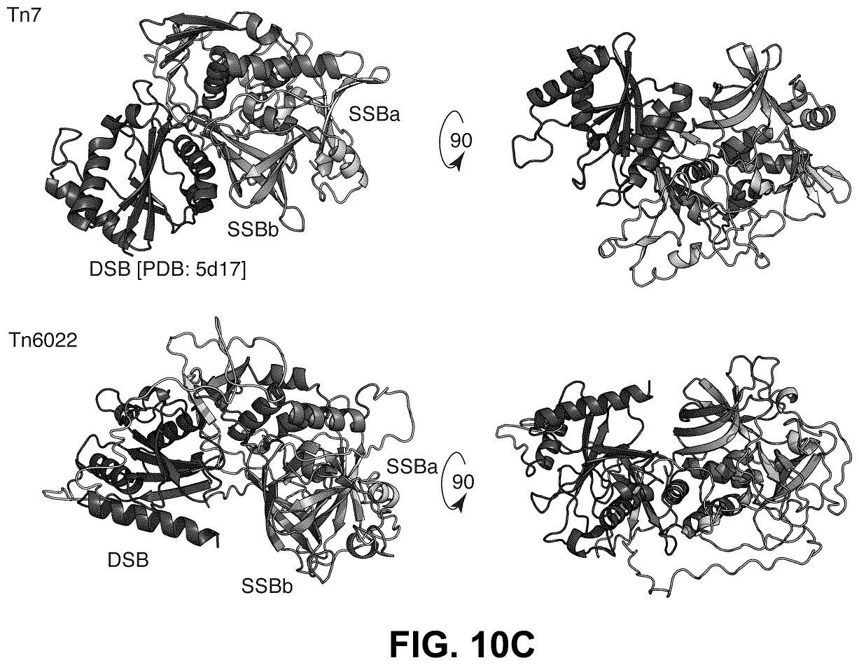

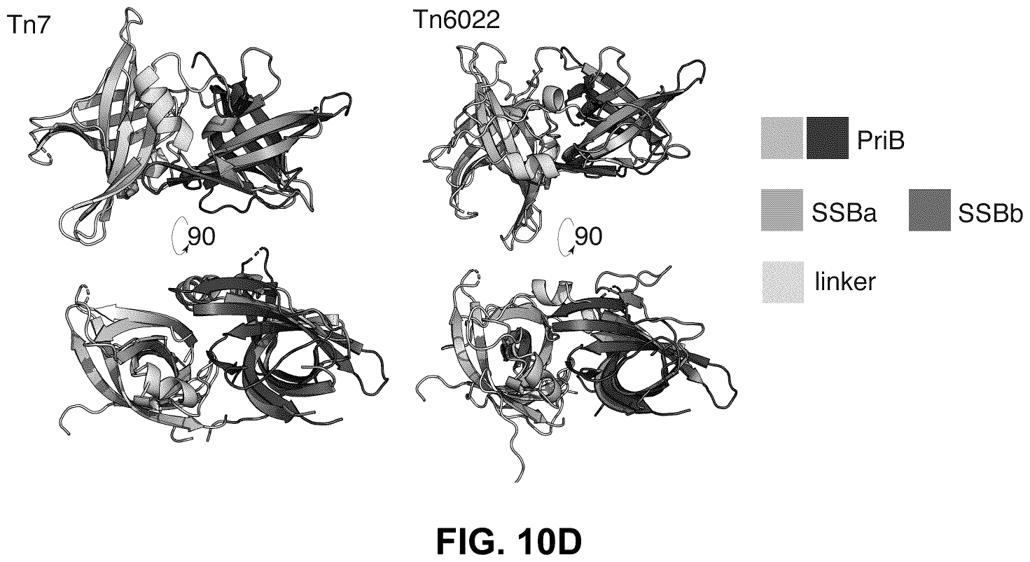

Apart from the dual TniQ-TnsD loci, numerous Tn7-like transposons encompass TniQ and another, unrelated target selector, such as TnsE, the plasmid target selector (FIG. 9B). Applicant identified two distinct branches encoding TniQ and TnsE (FIG. 10A-10B). Although these TnsEs are highly divergent in sequence (less than 10% of sequence identity) (FIG. 10C), they are predicted to form closely similar structures based on Alphafold2 modeling. The N-terminal domain of TnsE binds dsRNA via a unique fold (Shi, Q. et al. Conformational toggling controls target site choice for the heteromeric transposase element Tn7. Nucleic Acids Res. 43, 10734-10745 (2015)), but the activity of this domain has not been explored. Using structural prediction and structural mining (Holm, L. Dali server: structural unification of protein families. Nucleic Acids Res. 50, W210-W215 (2022)) of the N-terminal region, Applicant found that it folds into two single-strand binding (SSB) domains related to PriB (FIG. 10D), a component of the bacterial primosome, which can bind both ssDNA and ssRNA and is involved in restarting replication at the fork (Fujiyama, S. et al. Structure and mechanism of the primosome protein DnaT-functional structures for homotrimerization, dissociation of ssDNA from the PriB. ssDNA complex, and formation of the DnaT ssDNA complex. FEBS J. 281, 5356-5370 (2014); Huang, C.-Y., Hsu, C.-H., Sun, Y.-J., Wu, H.-N. & Hsiao, C.-D. Complexed crystal structure of replication restart primosome protein PriB reveals a novel single-stranded DNA-binding mode. Nucleic Acids Res. 34, 3878-3886 (2006); Lovett, S. T. Filling the gaps in replication restart pathways. Mol. Cell 17, 751-752 (2005)). Such structural similarity with PriB suggests that TnsE might have been co-opted from a system that functions at the replication fork. The domain architecture of TnsE has features including a dsDNA binding domain and a domain predicted to bind ssRNA or DNA, suggesting it targets the lagging strand of replication. Thus, TnsE is likely to specifically target replication forks of conjugative plasmids, further highlighting the remarkable diversity of target selectors co-opted by Tn7-like transposons.

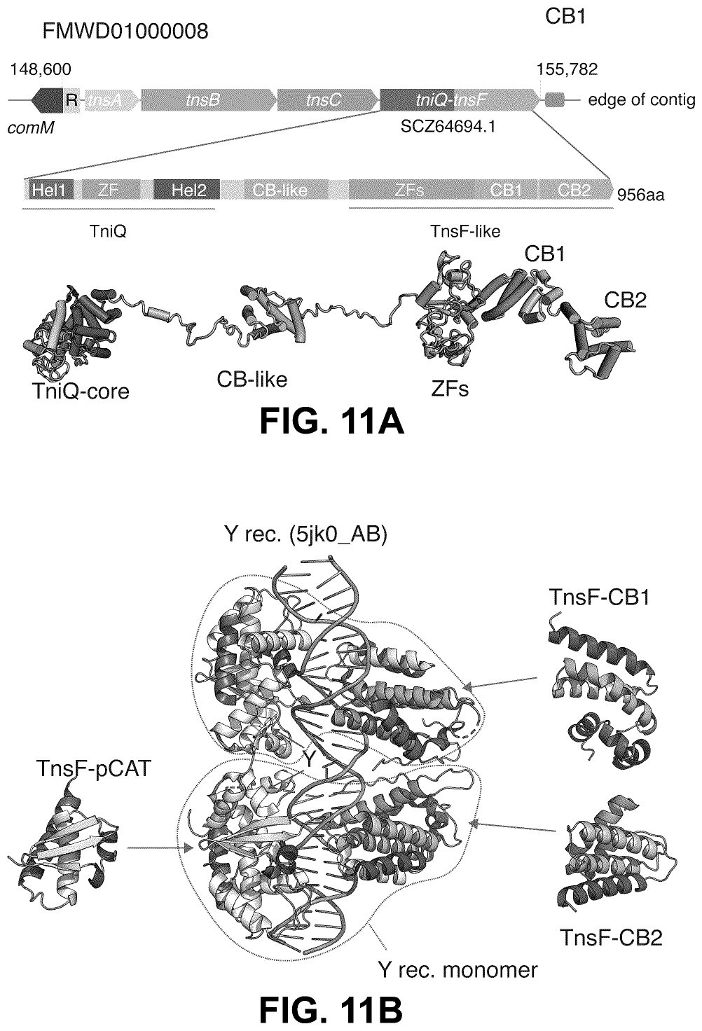

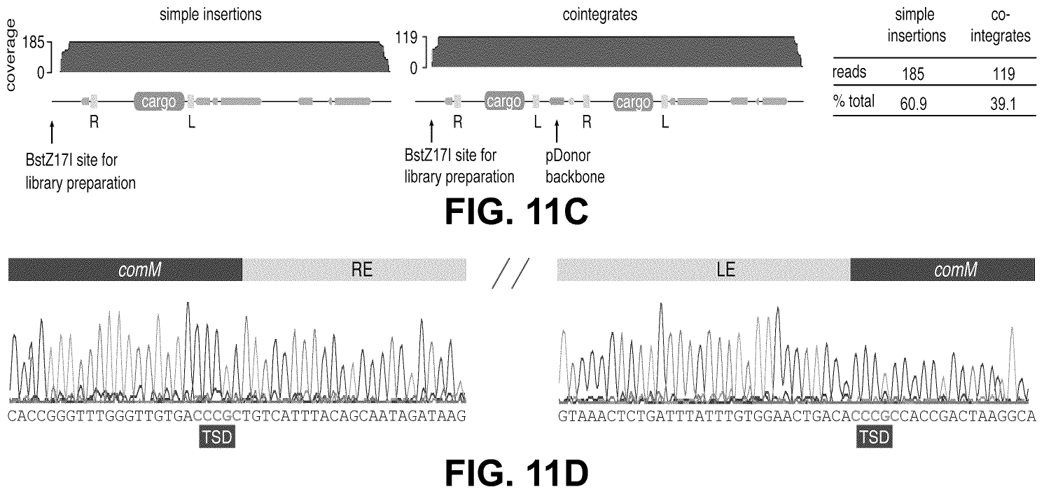

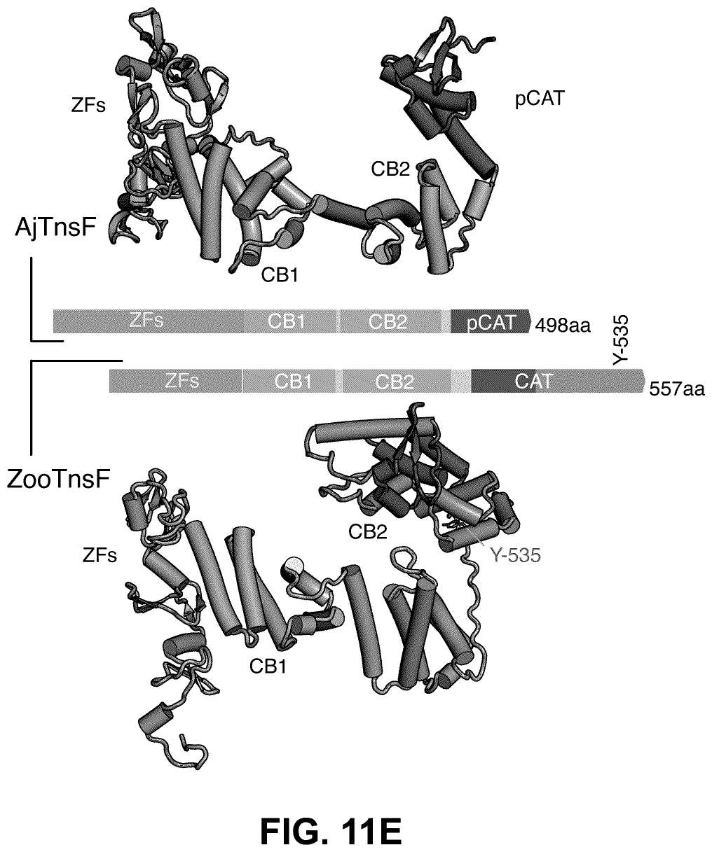

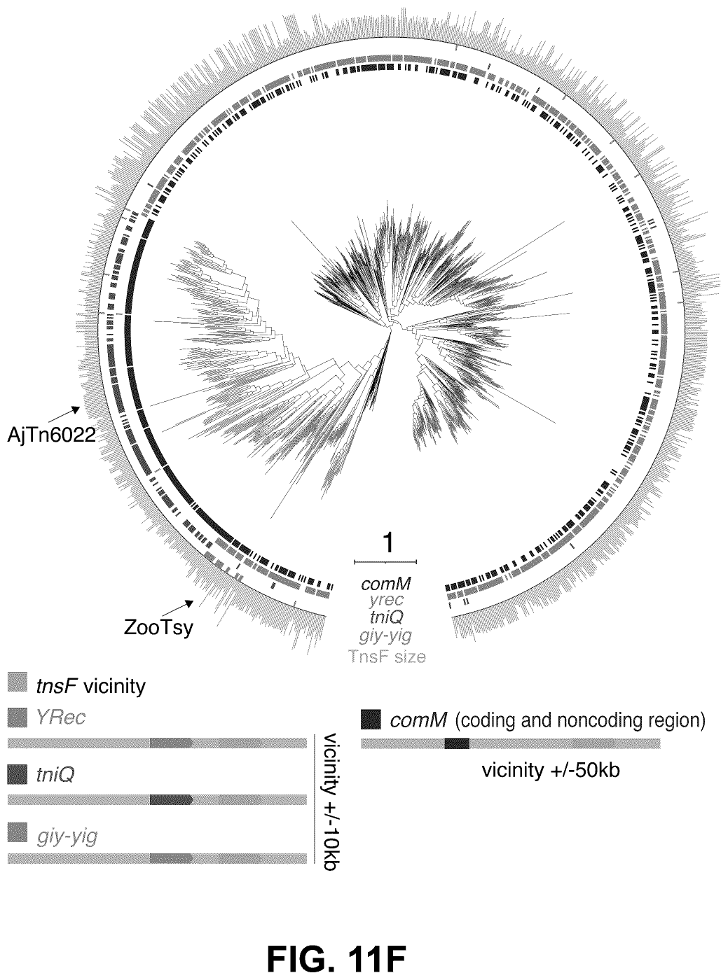

To search for additional target selectors, Applicant focused on genes operonized with TniQ/TnsD and conserved in multiple nodes (FIG. 9D). The most common candidate gene (121 groups of loci), found in the Tn6022 family of Tn7-like transposons, encodes an uncharacterized protein of 498aa, which forms a putative operon with a TniQ of 369aa and is denoted TnsF (previously annotated as orf3 or thif (Petassi, M. T., Hsieh, S.-C. & Peters, J. E. Guide RNA categorization enables target site choice in Tn7-CRISPR-Cas transposons. Cell 183, 1757-1771.e18 (2020); Partridge, S. R., Kwong, S. M., Firth, N. & Jensen, S. O. Mobile genetic elements associated with antimicrobial resistance. Clin. Microbiol. Rev. 31, (2018)), see SI). Structural prediction with Alphafold2 of TnsF revealed a novel domain architecture including an N-terminal region containing multiple ZFs in the first 199aa, whereas the remaining ˜300aa exhibit significant structural similarity to the N- and C-terminal domains of the tyrosine recombinase superfamily member XerH (pdb: 5jk0 PMID: 28009253) (Dali score 3.9) (FIG. 4B). However, the chamber holding the tyrosine catalytic site is missing in TnsF (FIG. 11A-11B). Tyrosine recombinases typically contain an N-terminal DNA binding domain (CB domain (Smyshlyaev, G., Bateman, A. & Barabas, O. Sequence analysis of tyrosine recombinases allows annotation of mobile genetic elements in prokaryotic genomes. Mol. Syst. Biol. 17, e9880 (2021))) and a C-terminal catalytic domain (CAT) and dimerize or tetramerise on DNA during site-specific recombination. Both the N-and C-terminal domains of XerH interact with DNA in the crystal structure (FIG. 11A). TnsF is predicted to contain tandem domains (designated CB1 and CB2) structurally similar and, by inference, homologous to the CB of XerH (FIG. 11A-11B), suggesting these domains may impart the ability to interact with DNA. A beta sheet at the C-terminus of XerH maps to the last 70aa of TnsF (designated partial CAT or pCAT) (FIG. 4B) and corresponds to the DNA binding region of the recombinase domain of XerH (FIG. 11A) but lacks the helix K (Bebel, A., Karaca, E., Kumar, B., Stark, W. M. & Barabas, O. Structural snapshots of Xer recombination reveal activation by synaptic complex remodeling and DNA bending. Elife 5, (2016)). In XerH, the helix K contributes substantially to its interaction with DNA, suggesting that the pCAT domain of TnsF might have lost DNA binding activity but gained the ability to interact with TniQ. Indeed, based on an Alphafold2 multimer model, TnsF is predicted to interact with TniQ through its C-terminal region (FIG. 4B). Additionally, Applicant found one case where the N-terminal region of TnsF is fused to the C-terminal region of TniQ (NCBI accession: SCZ64694). This fusion protein lacks the full catalytic domain but contains an additional CB-like domain within a linker between TniQ and TnsF.

The Tn6022 transposons also encode tnsE on the opposite strand, suggesting they can jump to conjugative plasmids, whereas TniQ and TnsF are likely involved in target selection within the bacterial chromosome. Together, these data suggest that TnsF is a novel target selector protein and that TniQ of Tn6022 serves as a hub that bridges TnsF, which binds directly to the attachment site, with the transposition machinery.

### TnsF is a Bona Fide Target Selector

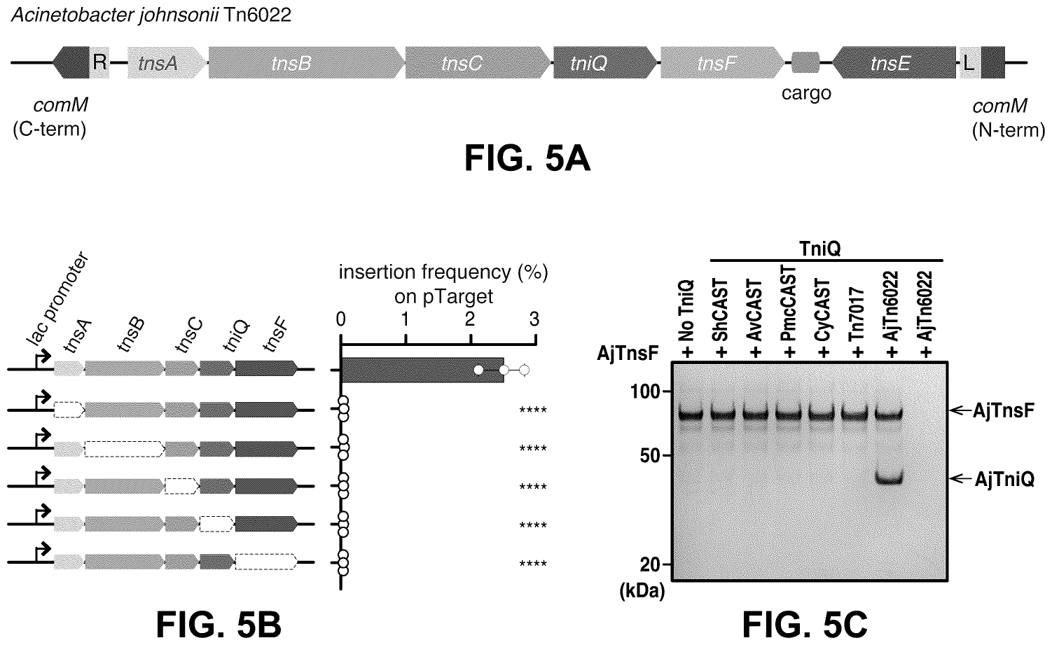

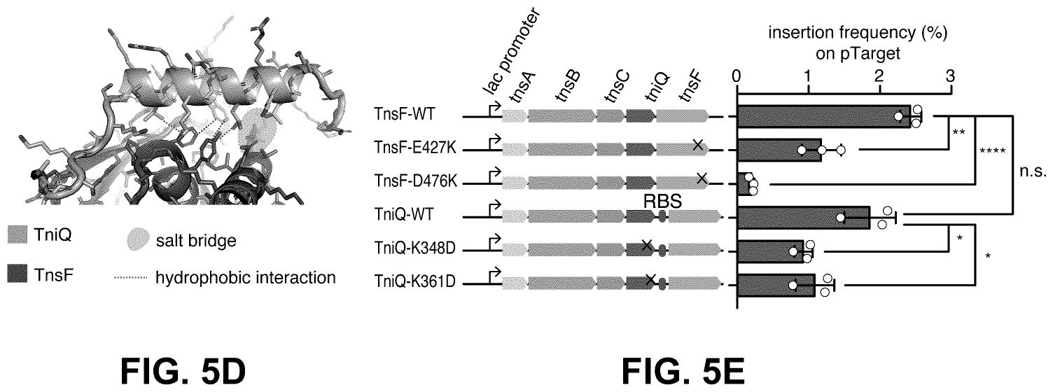

To experimentally test the predicted target partner selector role of TnsF, Applicant focused on the Tn6022 transposon. Tn6022 encodes TnsA, TnsB, TnsC, TniQ, TnsF, and TnsE and is inserted in the comM gene, which encodes a Mg chelatase-containing protein (FIG. 5A). Applicant reconstituted Acinetobacter johnsonii Tn6022 (hereafter AjTn6022) in E. coli. Applicant determined the ends of the transposon and cloned the left and right ends into a pDonor plasmid with a kanamycin resistance gene as a cargo. Applicant also cloned a 100-bp fragment (50 bp upstream and 50 bp downstream of the insertion site) of the AjcomM gene into a pTarget plasmid. These plasmids were co-electroporated into E. coli with a pHelper plasmid (bearing tnsA, tnsB, tnsC, tniQ, and tnsD. To determine the structure of the insertion, long read, amplification-free nanopore sequencing was performed. Applicant found simple insertions (60.9% of insertions) and co-integrate insertions (39.1% of insertions) (FIG. 11C), and confirmed the presence of target site duplications (TSDs), a signature of Tn7-like transposition (FIG. 11D). To determine whether all Tns proteins including TnsF are essential for transposition into the comM gene, Applicant generated pHelper variants lacking each Tns protein component and repeated the transposition assay. AjTn6022 achieves transposition at 2.4% efficiency, and removal of any Tns protein (A, B, C, Q, or F) impaired transposition, as quantified by ddPCR (FIG. 5B).

To confirm the prediction that TnsF and TniQ interact, Applicant purified TnsF and TniQ proteins from AjTn6022, and tested their interaction in vitro. Applicant showed that the TnsF interacts specifically with AjTniQ (and not with TniQ proteins from ShCAST, AvCAST, PmcCAST, CyCAST, or Tn7017) (FIG. 5C). The docking model of AjTnsF: AjTniQ predicts an interaction between the C-terminal helix of TniQ and the pCAT region of TnsF held by hydrophobic interactions and salt bridges (FIG. 5D). TniQ from other systems lack this helical region, which may explain why they do not interact with AjTnsF. Mutants designed to disrupt the predicted salt bridges (TnsF E427K/D476K and TniQ K348D/K361D) abrogated or substantially reduced transposition (FIG. 5D), supporting the hypothesis that this Aj-specific region of TniQ is important for interaction with TnsF.

Identification of Tsy, a Non-Th7 Transposon that Uses Task for Target Selection

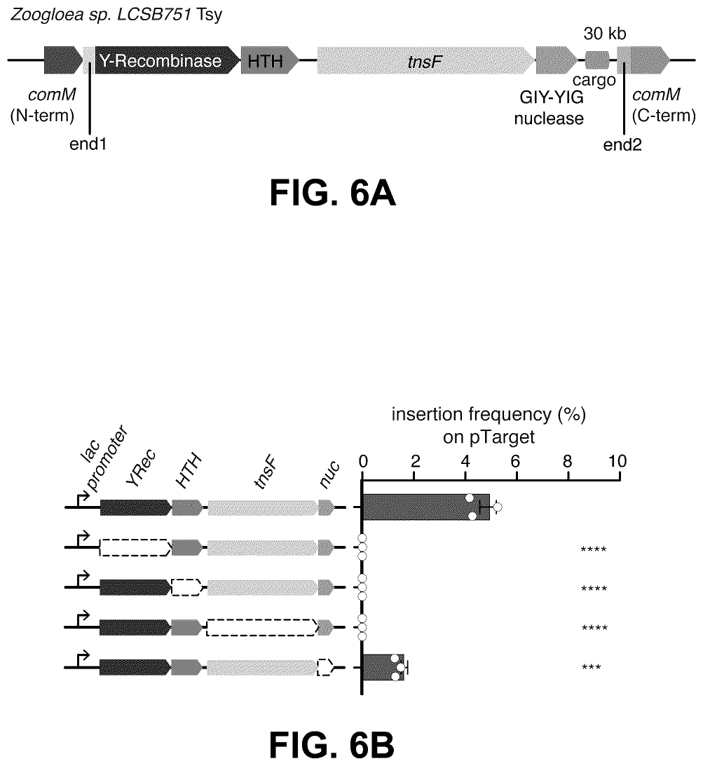

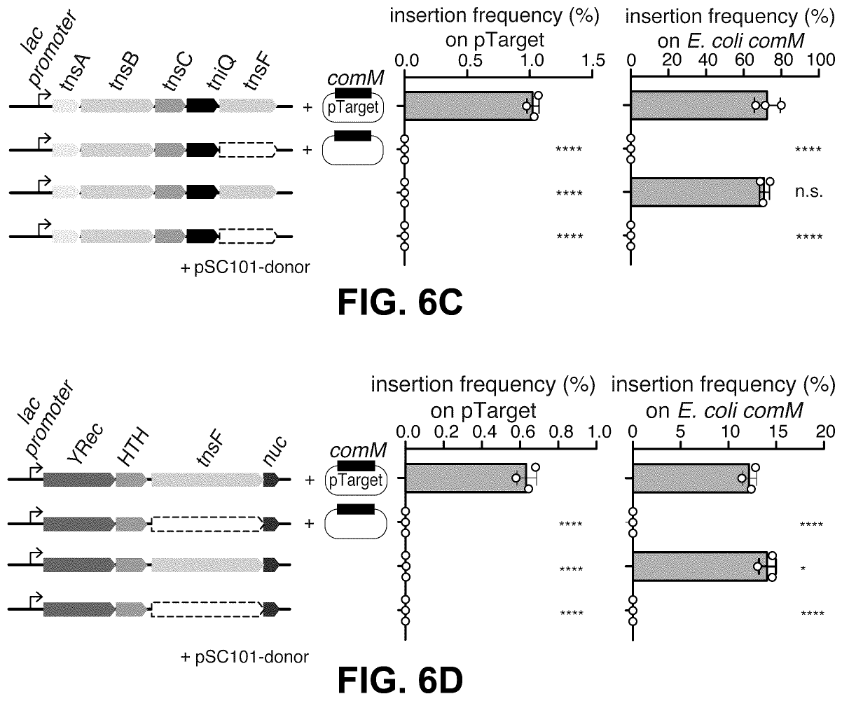

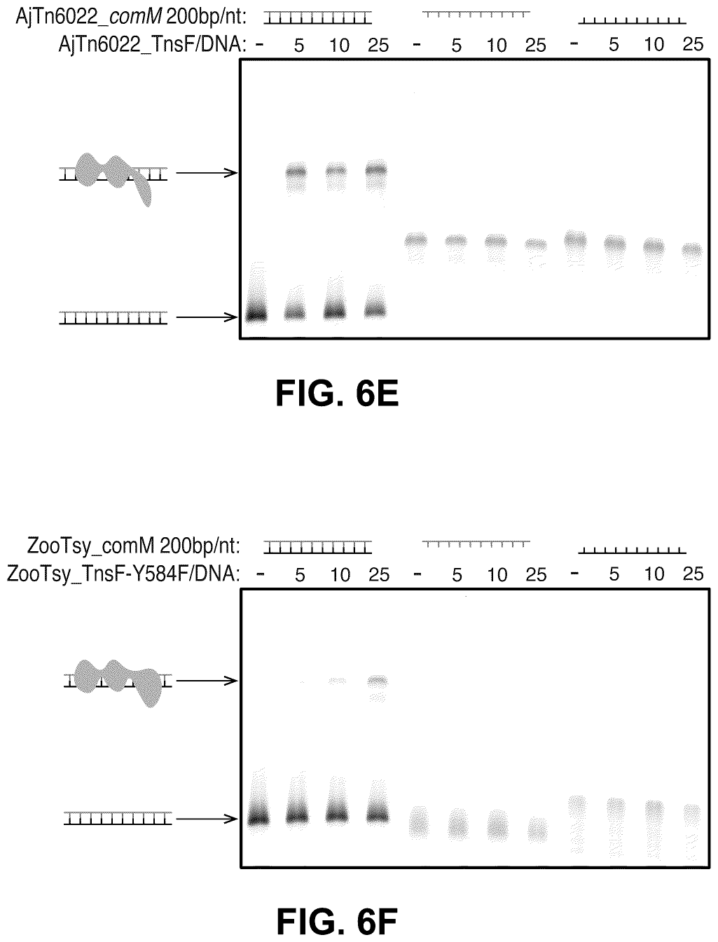

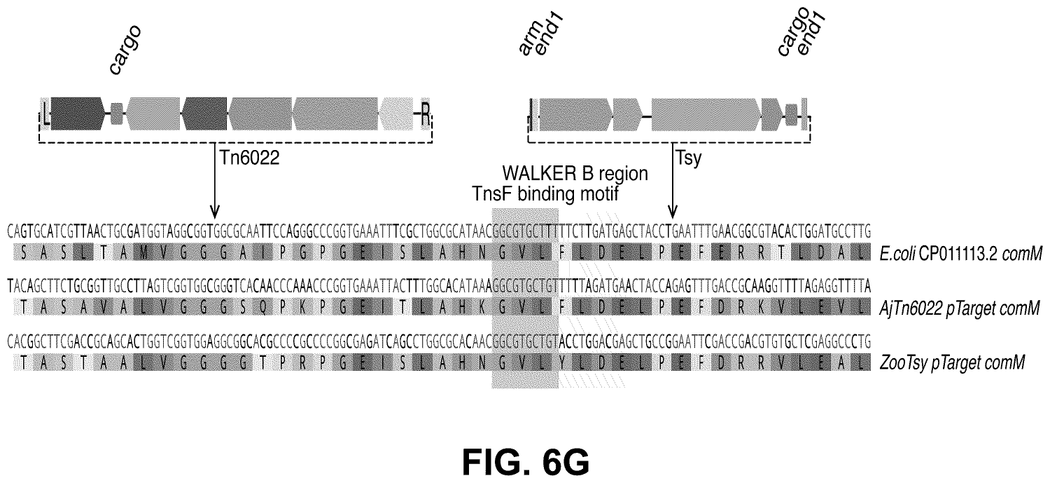

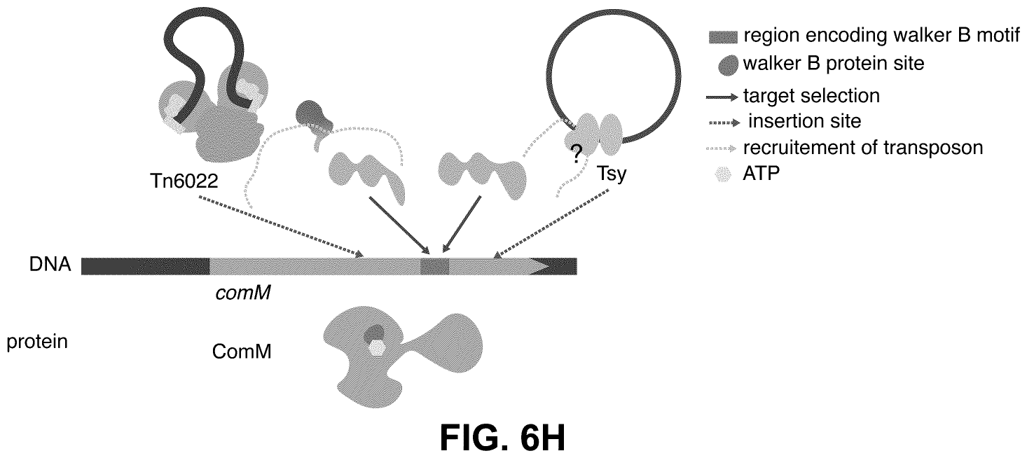

To gain further insight into the role of TnsF as a target selector, Applicant searched for TnsF homologs in genomic databases. Applicant selected 1,099 non-redundant TnsF homologs, including Tn6022 TnsF and a homolog of TnsF (referred to as TnsF-like protein) that harbors a predicted active catalytic domain (FIG. 11A-11B). Although Applicant also detected more remote structural homologs of TnsF harboring tandem CB and CAT domains, these homologs did not have ZF N-terminal regions, so Applicant did not focus on them (FIG. 11E). Applicant built a phylogenetic tree from the TnsF and TnsF-like proteins (FIG. 11F) and mapped on it conserved genes located in the vicinity of tnsF and tns/-like genes. The Tn7 TnsF forms a distinct clade and apparently evolved from TnsF-like proteins with an active catalytic domain. The genomic vicinity of these TnsF-like proteins lacks Tn7 components but instead includes upstream genes encoding a tyrosine recombinase (YRec) and a small HTH domain protein as well as a downstream gene encoding a GIY-YIG nuclease (present only in the branch close to Tn7) (FIGS. 6A and 11F). Although inverted repeats or any canonical ends in these loci could not be detected, the presence of comM fragments, namely, the 5′-terminal portion located upstream of YRec and the 3′-terminal portion located downstream of the predicted operon was detected (FIG. 6A). The downstream portion of the comM gene is in some cases located after several additional genes, which likely represent transposon cargo. These features suggest that this locus is a distinct transposon and that the TnsF-like protein recognizes comM, similarly to TnsF of Tn6022.

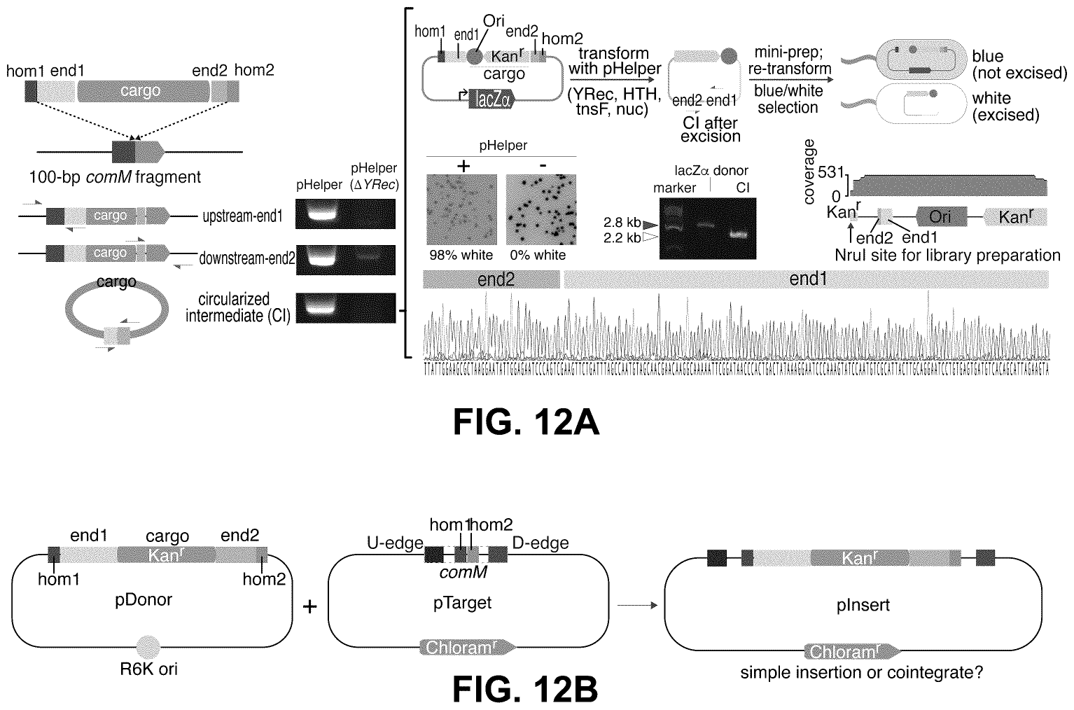

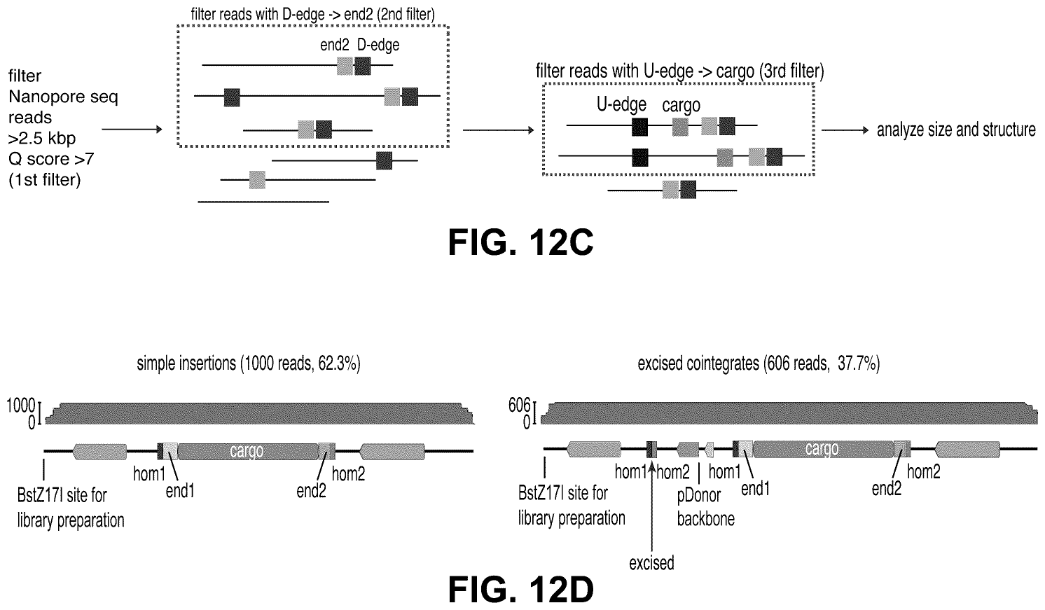

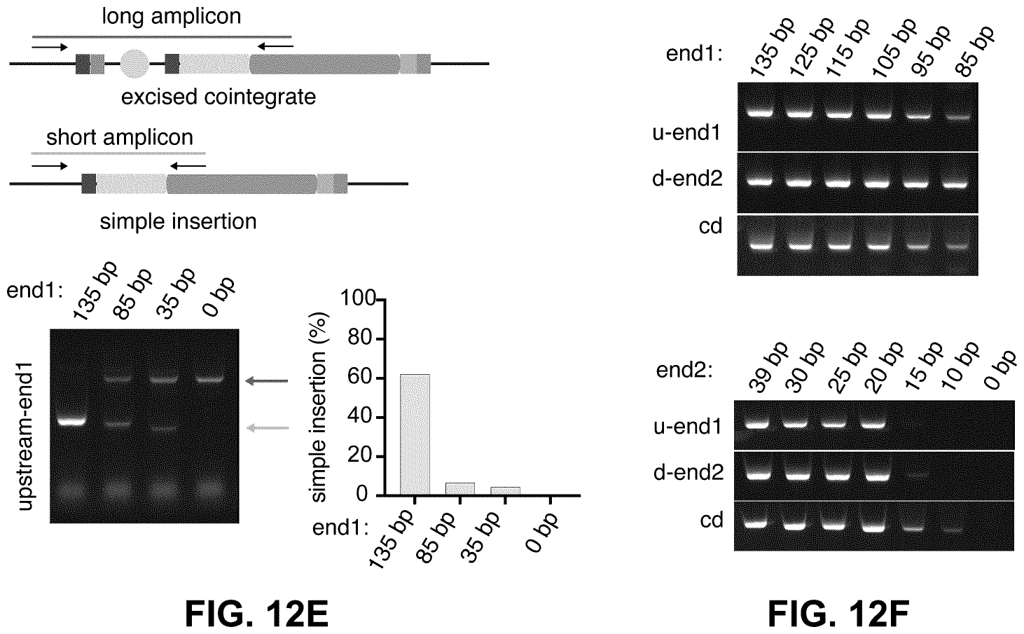

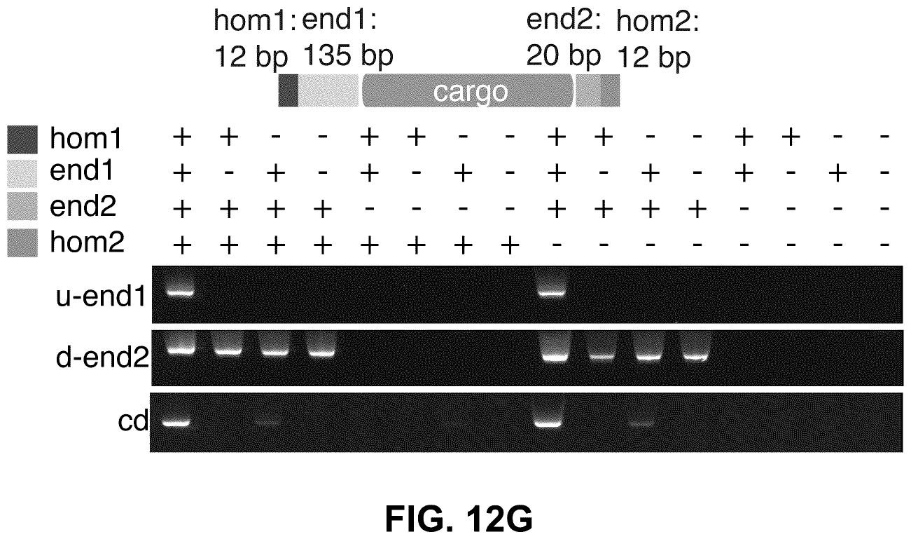

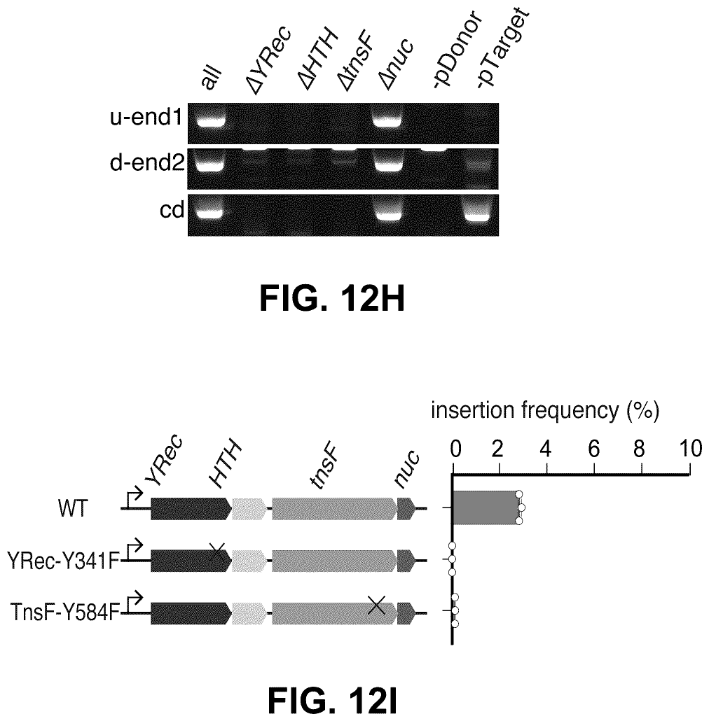

To test the function of this TnsF-like protein, Applicant experimentally characterized this mobile element (designated Tsy (Target Selector based on tYrosine (Y) recombinase). The system from Zoogloea sp. LCSB751 (hereafter ZooTsy) was reconstituted in E. coli. To assess ZooTsy transposition, Applicant cloned 135 bp and 39 bp of each transposon endl (comM 5′-terminal portion side) and end2 (comM 3′-terminal portion side) with 12 bp of homology arm extensions into an R6K origin pDonor plasmid with a kanamycin resistance gene as a cargo (FIG. 12A-12B). A Tsy attachment site (a 100-bp fragment with 50 bp upstream and 50 bp downstream of the Zoogloea sp. comM insertion site) was also cloned into a pTarget plasmid (FIG. 12A-12B). These plasmids were co-electroporated into E. coli with a pHelper plasmid (bearing YRec, HTH, tns, and the GIY-YIG nuclease, nuc). Applicant detected transposition into pTarget and observed circular intermediates (CIs) derived from pDonor by PCR in a YRec-dependent manner (FIG. 12A), as previously demonstrated for various transposons encoding tyrosine recombinases (Rubio-Cosials, A. et al. Transposase-DNA complex structures reveal mechanisms for conjugative transposition of antibiotic resistance. Cell 173, 208-220.e20 (2018)). To further validate this, Applicant established an assay to isolate and confirm the structure of the CI derived from pDonor. Applicant created a new pDonor that contains the ColEl origin of replication and kanamycin resistance gene as cargo as well as lacZα but no other origin of replication. Upon circularization, this pDonor will lose lacZα and can be isolated from white colonies by traditional blue-white screening (FIG. 12A). Using this assay, Applicant obtained white colonies (98% of total) in a pHelper-dependent manner after re-transformation with the extracted plasmids and successfully isolated a smaller plasmid which had lost the 0.6-kb backbone region of pDonor (FIG. 12A). Applicant confirmed the smaller plasmid as a CI by nanopore long-read sequencing and observed the connected endl and end2 junction by Sanger sequencing (FIG. 12A). To determine the structure of the ZooTsy insertions, Applicant performed nanopore long-read sequencing and found simple insertions (62.3% of total insertions) as well as cointegrates (37.7% of total insertions) in which an upstream copy of ZooTsy was excised before resolution (FIG. 12C-12D).

To narrow down the requirements for transposition, variants of the cargo with serial deletions of end1 from 135 bp to 0 bp were generated, finding that truncation of this end gradually decreased the rate of simple insertions to zero (FIG. 12E-12F). By contrast, only 20 bp at end2 are required for the transposition (FIG. 12F). By systematically combining these optimized parameters, Applicant found that 12 bp of homology arm 1 (hom1), 135-bp end1, and 20-bp end2 are sufficient for ZooTsy to achieve transposition (FIG. 12G).

To determine the genetic requirements for ZooTsy transpositions in E. coli, a series of pHelper plasmids with deletions of each gene were constructed. ZooTsy achieves transposition at 5.0% efficiency, and removal of any component (YRec, HTH, TnsF, or GIY-YIG nuclease) from the system impaired transposition, as quantified by ddPCR for the upstream-end1 junction formation (FIGS. 6B and 12H). The GIY-YIG nuclease, however, is not essential for transposition, which is supported by the identification of Tsy relatives lacking this component (FIG. 11E). Applicant also confirmed that the tyrosine recombinase catalytic activities of both YRec and TnsF are essential for transposition (FIG. 12I).

Tnsk Targets a Conserved Region of comM

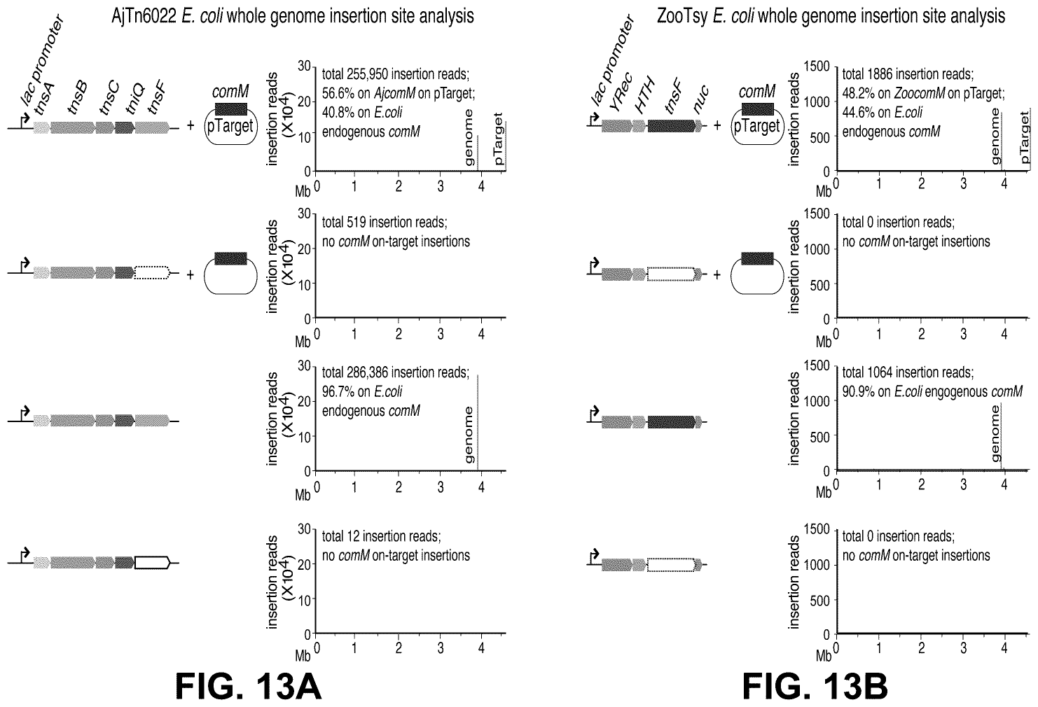

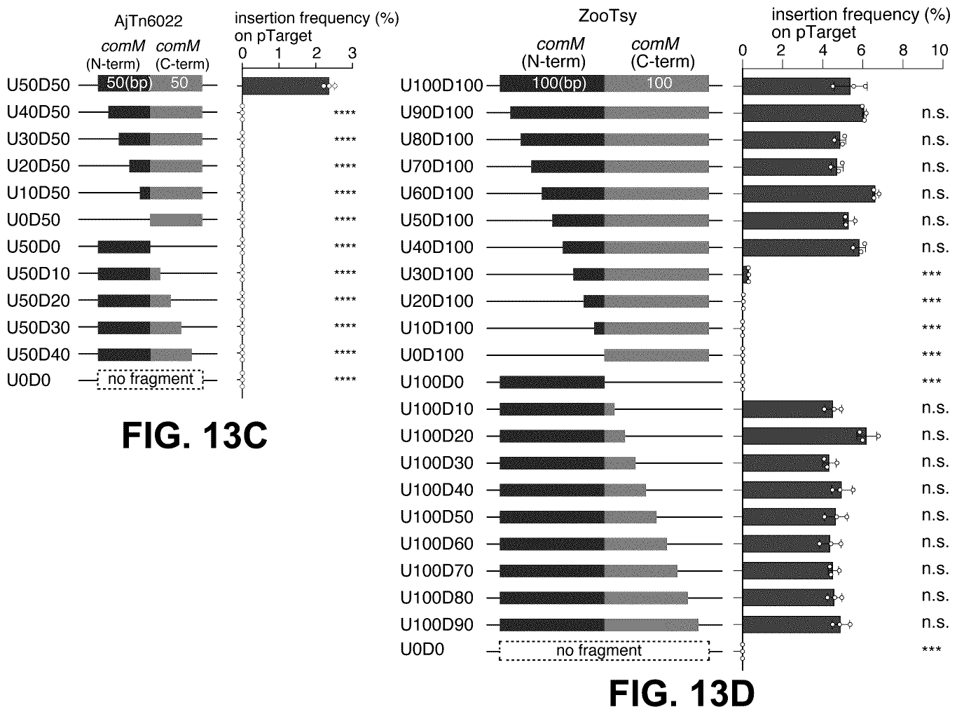

Both AjTn6022 and ZooTsy use TnsF to target comM of their respective hosts. ComM has been reported to facilitate recombination of sequences acquired by transformation (Nero, T. M. et al. ComM is a hexameric helicase that promotes branch migration during natural transformation in diverse Gram-negative species. Nucleic Acids Res. 46, 6099-6111 (2018)), and disruption of the comM gene by transposon insertion could inactivate this functionality, limiting transformation of other MGEs. To explore comM targeting in greater depth, Applicant sought to compare TnsF-mediated targeting of this gene by AjTnsF and ZooTnsF. Using heterologous E. coli expression systems, Applicant looked for AjTn6022 and ZooTsy targeting of genomic comM, finding that they can both target E. coli endogenous comM in addition to their respective comM sites on pTarget, highlighting the broad recognition of comM by both TnsFs (FIG. 6C-6D). To determine the specificity of comM gene targeting by TnsF, Applicant performed tagmentation-based tag integration site sequencing (TTISS) (Schmid-Burgk, J. L. et al. Highly Parallel Profiling of Cas9 Variant Specificity. Mol. Cell 78, 794-800.e8 (2020)). For AjTn6022, in the absence of pTarget, 96.7% of insertions at the E. coli endogenous comM locus were observed; in the presence of pTarget, 56.6% of insertions were on pTarget and 40.8% were on the genome comM (97.4% on-target in total) (FIG. 13A). For ZooTsy, similarly high levels of specificity were observed, indicating that TnsF is highly selective for comM (FIG. 13B). Applicant confirmed both TnsF proteins bind 200-bp dsDNA fragments corresponding to their respective comM target sequences (FIG. 6E-6F).

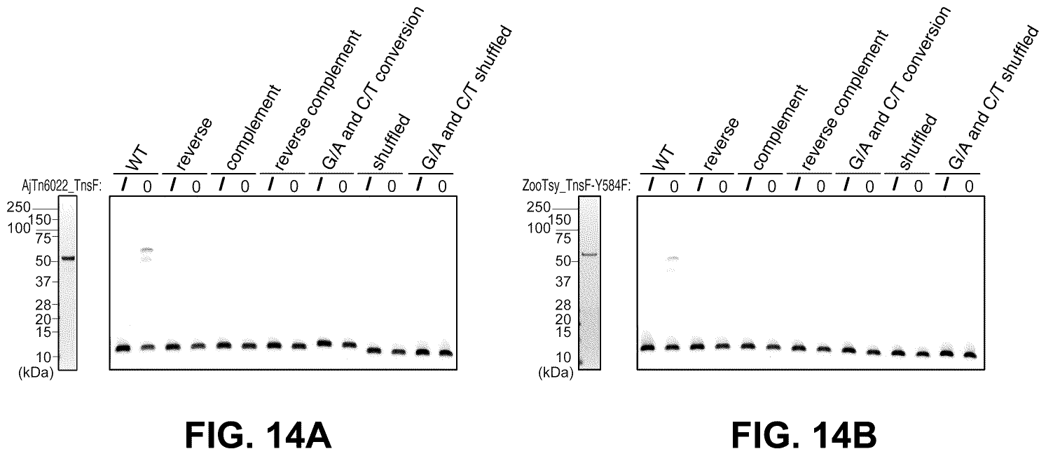

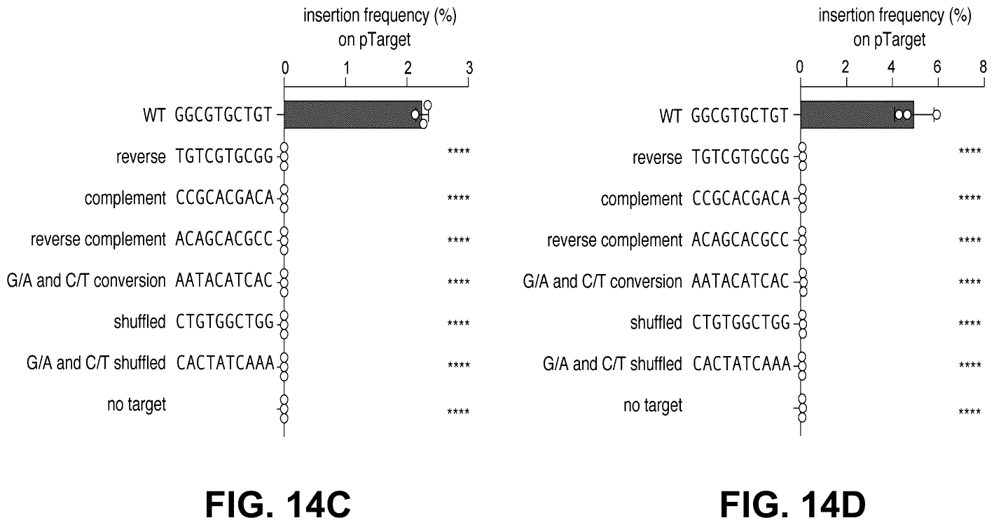

To further characterize the attachment sites of these proteins, Applicant constructed additional pTarget variants with different lengths of comM gene fragments to map the target-site specificity of both TnsFs with greater resolution. For AjTn6022, deletion of the 40-50 bp-either upstream or downstream sequences of the insertion site substantially reduced transposition, indicating that the insertion site is within the TnsF attachment site (FIG. 13C). For Tsy, Applicant found the attachment site is within a 40 bp-upstream region from the insertion site (FIG. 13D). Mapping these refined target sites on the respective comM genes revealed they overlap with a conserved 10-bp region within the WalkerB motif (a conserved ATPase motif (Iyer, L. M., Leipe, D. D., Koonin, E. V. & Aravind, L. Evolutionary history and higher order classification of AAA+ATPases. J. Struct. Biol. 146, 11-31 (2004)) of the comM protein (FIG. 6G). Applicant confirmed this 10-bp region is necessary for both AjTnsF and ZooTnsF binding (FIG. 14A-14B). Furthermore, Applicant found that mutating this region abolished transposition activity for both AjTn6022 and ZooTsy (FIG. 14C-14D). Targeting the Walker B motif of comM might provide a natural conserved anchor for TEs to spread across species, paralleling Tn7 targeting of the catalytic site in glmS. Together, these results demonstrate that TnsF is a novel target selector found in two distinct transposons and highlights the ability of Tn7-like transposons to co-opt target selectors from various systems and adapt them by abrogating their original functions, such as recombinase/nuclease activity.

### Supplementary Information

### TnsC Phylogeny Analysis

Applicant selected loci using the following criteria: at least 2 genes, one of which is a tns C homolog and the other is a homolog of another Tn7 component (tnsA, tnsB, tniQ or tnsD (tniQ/tnsD, or tnsk:), and the presence of at least two closely spaced codirectional Tn7 components comprising a predicted operon (See Methods). Applicant obtained 80,028 loci meeting these criteria, from which 6,384 non-redundant tns C homolog representatives (<80% sequence identity) were extracted and a phylogenetic tree was built from them (FIG. 2). Applicant mapped onto the tree known target selectors tniQ/tnsD and tnsE and found they are present in the Tn7 clades (the isolated occurrences of these components in the MuB branch are instances of Tn7 transposons located near MuTn). TnsE is detected in 2 distant branches, one encompassing the canonical E. coli Tn7 and another the Tn6022 transposon. These two groups of transposons harbor distinct InsEs distantly related and only detectable by profile profile comparison (hhpred probability >=90) (Söding, J., Biegert, A. & Lupas, A. N. The HHpred interactive server for protein homology detection and structure prediction. Nucleic Acids Res. 33, W244-8 (2005); Steinegger, M. et al. HH-suite3 for fast remote homology detection and deep protein annotation. BMC Bioinformatics 20, 473 (2019)), suggesting an ancient origin of tnsE in the Tn7 family.

### Search for CAST Systems

From TnsC phylogenetic tree analysis (FIG. 2), it was found that CAST systems are spread around the tree: CAST V-K forms one clade, suggesting a single ancestral capture of Cas12k, CAST I-B is found in two distinct clades respective to their subtype (1 and 2), suggesting two distincts transposons independently captured a Cascade I-B system (Saito, M. et al. Dual modes of CRISPR-associated transposon homing. Cell 184, 2441-2453.e18 (2021)), and CAST I-F forms its own clade. However, Tn7017, a CAST I-F system that uniquely harbors a dual tniQ-tnsD and uses TnsD for protein-mediated homing (in contrast to other CAST I-F systems that use a dedicated spacer for RNA-guided transposition (Petassi, M. T., Hsieh, S.-C. & Peters, J. E. Guide RNA categorization enables target site choice in Tn7-CRISPR-Cas transposons. Cell 183, 1757-1771.e18 (2020)) is located in a side branch containing tns ('s with dual tniQ-tnsD and sparsely populated with Cascade I-B systems. This separation of CAST I-F and Tn7017 suggests either an ongoing gain or loss of Cascade I-F or a trans partnership with a Cascade system located outside the transposon border.

Several groups of loci in the MuB clade harbor associations with Cascade including I-E Cascade, in agreement with a previous report (Faure, G. et al. CRISPR-Cas in mobile genetic elements: counter-defence and beyond. Nat. Rev. Microbiol. 17, 513-525 (2019)), however, the absence of detectable ends and the presence of the cas3 nuclease gene suggest that these Cascade systems are not likely to be involved in RNA-guided transposition with MuA/MuB components and therefore are unlikely to be target selectors.

Analysis of the Genes Operonized with tnsC's

To search for new target selectors, Applicant first looked at non-Tn7 components (i.e., genes other than tnsA, tnsB, tnsC′, tniQ/tnsD, or tnsk) that are conserved in the vicinity of closely related tns ('s (defined as branches that group together to form a subtree) and operonized with it. It was observed that the entire MuB clade has a conserved and operonized gene downstream, which is a known component of the Mu phage. The Tn7 TnsC clade contains a few branches harboring potential candidates, but manual inspection of these candidates revealed that they are remote homologs of TniQ and TnsD that were not detected automatically, highlighting the diversity of these proteins. No new components directly operonized with ins C were found.

Structural analysis of dual TniQ/TnsD

From the structural prediction of CAST dual TniQ-TnsD and Tn7 TnsD Applicant found the core region of TniQ consists of a helical domain (HelD, a zinc finger (ZF), a connector helix, and another helical domain (Hel2) (FIGS. 3A and 9A). Hel2 in TnsD of Tn7, TniQ and TnsD of CAST I-B, and TnsD of CAST I-D fold into an HTH domain that is structurally similar to an XRE family transcription factor (TF) (FIG. 9A). Hel2 in TniQ of CAST I-D is composed of 3 helices that fold into a smaller domain that seems to be a partial version of CAST I-D TnsD. Hel2 in TniQ and TnsD of Tn7017 folds into a large helical domain distinct and larger than other Hel2 domains (FIG. 9A).

The structure of the Cascade I-F-TniQ complex from Tn6677 CAST I-F (a single TniQ CAST I-F) revealed that TniQ dimerizes head to tail via an HTH domain located downstream of Hel2 (Halpin-Healy, T. S., Klompe, S. E., Sternberg, S. H. & Fernández, I. S. Structural basis of DNA targeting by a transposon-encoded CRISPR-Cas system. Nature 577, 271-274 (2020)) and that this dimer interacts with Cas6 (as well as Cas7 and the guide RNA) through a loop in the Hel2 domain, suggesting that Hel2 bridges TniQ to the Cascade complex. Hel2 domains from TniQ and TnsD of CAST I-B2 and CAST I-D share a similar fold. However, the Hel2 domain in CAST I-D TniQ shows the most pronounced differences compared to both CAST I-D TnsD and TniQ/TnsD of CAST I-B2. This Hel2 domain seems to be truncated, which distorts the structure and likely the function of the HTH fold (FIG. 9A). Finally, CAST V-K TniQ is the smallest protein of this family, containing only the Hell and ZF domains and lacking the connector helix and the Hel2 domain (Querques, I., Schmitz, M., Oberli, S., Chanez, C. & Jinek, M. Target site selection and remodelling by type V CRISPR-transposon systems. Nature 599, 497-502 (2021)).

The C-terminal extensions of TnsD proteins show broad diversity in sequence, length, and predicted structure (FIG. 3A). For example, the C-terminal region of TnsD from Tn7 accommodates three HTH domains (distinct from the one in TniQ from Tn7017, which is involved in dimerization) interspaced with disordered linkers (FIG. 3A). Three HTH domains are similarly predicted to exist in the C-terminal regions of the TnsD proteins of CAST I-B and I-D. Other TnsD proteins contain even larger C-terminal regions: TnsD of Tn7017 contains an additional domain composed of an alpha helix and beta sheet, and TnsD of CAST I-B1 contains an additional domain composed of a beta sheet with an unknown function. These domains are likely involved in additional interactions during transposition that remain to be explored.

### Analysis of TniQ/TnsD Size

TniQ/TnsD size distribution shows 4 distinct groups (FIG. 9C). Group 1 (g1) encompasses proteins that are smaller than 220aa and are mainly seen in CAST V-K systems. The second group (g2) of proteins range from 220aa to 400aa and encode the TniQ core region with no or only a short C-terminal addition, which suggests they probably cannot recognize target sites alone and need a partner to perform target selection. The proteins from group 3 (g3), which are the most widespread, range from 400aa to 550aa and encode a C-terminal extension that is likely long enough to recognize target sites. Finally, group 4 (g4) encompasses proteins larger than 550aa and which harbor very large C-terminal regions, suggesting they bind longer target sites and/or encode additional functions.

### TniQ-TnsD Tandem

Applicant identified 5072 TniQ/TnsD protein in total in the vicinity of the Tn7 tnsc representatives. 2905 loci encode a single TniQ/TnsD, while 998 loci have a dual TniQ-TnsD, 33 have 3 TniQ/TnsD, 17 have more than 3 TniQ/TnsD. Manual inspection indicates the loci with more than 2 TniQ-TnsD are usually involving partial TniQ maybe results from degradation of TniQ or insertion of another Tn7 where other transposases would be localized beyond the edge of the contigs. For the TniQ-TnsD tandem analysis, for loci encoding more than 2 TniQ/TnsD Applicant selected the two largest TniQ/TnsD and considered these systems as TniQ-TnsD tandem systems. Thus, Applicant considered a total 1048 tandem TniQ-TnsD systems and considered 1029 of them from which the sequences were not discarded during the alignment constructions (see Methods).

Dual TniQ-TnsD loci often harbor at least one protein larger than 400aa (FIG. 9C), suggesting that it is a direct target selector. Typically, one of the two proteins is larger than the other, with the most common architecture being one protein from g2 and the other protein from g3. Such an architecture could provide systems with multiple options for target selection: either directly via TnsD or indirectly via TniQ interacting with an external target selector.

To explore the origin of dual TniQ-TnsD systems, a phylogenetic tree of TniQ/TnsD using the core region (FIG. 9C) was built and tandem TniQ/TnsD proteins (encoded in the same locus) were mapped using connecter lines that link one TniQ/TnsD leaf to the other TniQ/TnsD leaf when they belong to the same locus. Using statistical comparison of branch distances and visualization of these connectors, Applicant found that the dual TniQ-TnsD likely emerged multiple times and independently via duplication of a single TniQ/TnsD (FIG. 9A-9D) ruling out an origin whereby an ancestral TniQ/TnsD would give rise to all dual TniQ-TnsD systems (p-value=2.4e-236). The duplication of TniQ/TnsD in situ might ensure compatibility with the other transposase components while opening up the possibility of acquiring different target selectors, either direct (encoded in the C-terminal region of TnsD) or via a partnership (through interaction with TniQ. This scenario may explain the frequency of duplication events. Consistent with this idea, Applicant found only a few examples of dual TniQ-TnsD loci containing very distant proteins, as would be expected to arise from the exchange of TniQ/TnsD. (FIG. 9B).

### Structural Comparison of Tandem TniQ/TnsD CAST Systems

The presence of dual tniQ/tnsD genes in a variety of Tn7 loci motivated Applicant to look more deeply at these proteins to better understand their roles in target selection. Mechanistic studies of CAST systems have shown that TniQ, which is smaller than TnsD, partners with Cascade to mediate RNA-guided transposition, whereas TnsD mediates protein-guided transposition (similar to its role in E. coli Tn7) (Saito, M. et al. Dual modes of CRISPR-associated transposon homing. Cell 184, 2441-2453.e18 (2021)). Applicant therefore first focused on TniQ and TnsD from these systems. Structural prediction was employed using AlphaFold2 (Evans, R. et al. Protein complex prediction with AlphaFold-Multimer. bioRxiv (2021) doi: 10.1101/2021.10.04.463034; Jumper, J. et al. Highly accurate protein structure prediction with AlphaFold. Nature 596, 583-589 (2021); Mirdita, M. et al. ColabFold: making protein folding accessible to all. Nat. Methods 19, 679-682 (2022)) to compare the domain organization of TniQ and TnsD variants encoded by E. coli Tn7, CAST I-B, CAST I-D, and Tn7017 (the dual TniQ-TnsD CAST I-F) (FIGS. 2 and 9A). Applicant identified a common core structure shared by CAST TniQ and TnsD of about 300 amino acids (aa). The CAST TniQ proteins are largely restricted to this core region, whereas the CAST TnsD and Tn7 TnsD proteins have a long C-terminal extension. Applicant hypothesized that this additional region in TnsD might confer target DNA recognition to enable protein-guided transposition. Accordingly, Applicant proposed that TniQ family proteins predicted to perform target selection be named TnsD, while the TniQ designation be reserved for those proteins that consist of the core region but cannot perform target selection without a partner. Applicant notes, however, that TniQ and TnsD seem to represent two ends of a continuum, which is spanned by proteins containing extensions of variable length (FIG. 9C), making it difficult to predict in some cases whether they bind directly to attachment sites or require a target selector partner. As a reflection of this continuum, Applicant refers to some proteins as TniQ/TnsD when the ability of the proteins to interact directly with a target site could not be predicted.

The core region of TniQ consists of a helical domain (HelD, a zinc finger (ZF), a connector helix, and another helical domain (Hel2) (FIGS. 2 and 9A). Even within this core region, there are notable differences in the TniQ and TnsD proteins found in different systems. For example, in some proteins, the Hel2 domain folds into an HTH domain that is similar to an XRE family transcription factor (TF), whereas in others, this domain has a unique fold (FIG. 9A). TniQ (from Tn6677 CAST I-F) interacts with Cas6 (as well as Cas7 and the guide RNA) through a loop in the Hel2 domain (Halpin-Healy, T. S., Klompe, S. E., Sternberg, S. H. & Fernández, I. S. Structural basis of DNA targeting by a transposon-encoded CRISPR-Cas system. Nature 577, 271-274 (2020)) suggesting Hel2 might bridge to Cascade and that the diversity of Hel2 might encode compatibility with distinct Cascades. TniQ from CAST V-K, which uses the Cas12k effector rather than Cascade, contains only the Hell and ZF domains, suggesting a different mode of target selector partner interaction, highlighting the flexibility of TniQ as an adaptor between target selection and transposase machinery.

The C-terminal extensions of TnsD proteins show even broader diversity in terms of sequence, length, and predicted structure (FIG. 9A). This region often folds into HTH domains (distinct from the one found in the core region) interspaced with disordered linkers. For example, three HTH domains are predicted to exist in the C-terminal regions of the TnsD proteins of CAST I-B and I-D suggesting similarities in the mechanism of recognition of the attachment site.

The structural analysis described herein suggests that the conserved core region of TniQ and TnsD likely mediates the recruitment of the transposon (via direct interaction with TnsC or via local conformational changes in the DNA near the target site), consistent with observations from the CAST V-K system (Querques, I., Schmitz, M., Oberli, S., Chanez, C. & Jinek, M. Target site selection and remodelling by type V CRISPR-transposon systems. Nature 599, 497-502 (2021); Schmitz, M., Querques, I., Oberli, S., Chanez, C. & Jinek, M. Structural basis for RNA-mediated assembly of type V CRISPR-associated transposons. bioRxiv (2022) doi: 10.1101/2022.06.17.496590). By contrast, the C-terminal region in TnsD recognizes the attachment site in protein-guided transposition (FIG. 9A). In CAST systems, the Cas effectors cooperate with TniQ proteins that lack the C-terminal extension, likely via the Hel2 domain. This raises the possibility that the TniQ core acquired the ability to bind other target selector proteins as well. Together, these results suggest that the presence of a small TniQ lacking a large C-terminal extension is a general hallmark of Tn7-like transposons that employ additional target selectors.

Analysis of the genes operonized with tniQs

In addition to instances of dual TniQ-TnsD loci, there are also systems that contain TniQ and another target selector from a non-TniQ family (e.g., TnsE). To search for non-TniQ family target selector partners of TniQ and independent target selectors, Applicant focused on genes operonized with TniQs with no or minimal C-terminal extensions (FIG. 9D). Applicant found several genes operonized with TniQ/TnsD that are conserved in multiple nodes. The 6 largest groups of candidates were selected based on the conservation of the operonized genes across several adjacent leaves in the TniQ tree. For example, Cascade I-F, a known target selector partner, forms a conserved pool of genes operonized with TniQ, validating the approach described herein (FIG. 9B). Applicant found 5 conserved candidates located in closely related branches of the TniQ/TnsD tree. The first candidate (comprising 6 groups of loci) encodes a protein with a weak relationship to the C-terminal region of Csn2, a protein found in Class 2 type II-A CRISPR-Cas systems that is linked to adaptation. The structural docking prediction indicates a potential interaction between the candidate and a region at the surface of TnsC distinct from where TniQ/TnsD is predicted to interact, suggesting this candidate is likely not performing target selection in partnership with TniQ/TnsD but rather independently (FIG. 9D). The second candidate is an unknown gene operonized with a g4 TnsD (found in 16 groups of loci) and does not seem to interact with TnsC or TniQ/TnsD (FIG. 9D), suggesting it is likely not involved in target selection. The third candidate (comprising 8 groups of loci) is a small gene sharing similarity with transcription factor (TF). It is located in between two tniQ/tnsD (encoding proteins of 500aa and 550aa) and is not predicted to interact with TniQ/TnsD or TnsC. Although this TF-like protein is likely not involved in target selection given the lack of interaction with TnsC or TniQ, TFs have been shown to regulate CAST I-F transposition (Petassi, M. T., Hsieh, S.-C. & Peters, J. E. Guide RNA categorization enables target site choice in Tn7-CRISPR-Cas transposons. Cell 183, 1757-1771.e18 (2020)) and are also found in CAST V-K systems. Thus, this TF could regulate the expression of TniQ/TnsD in this system. The fourth candidate (comprising 31 groups of loci) is a small gene with similarity to ParD-like proteins, which are the antitoxin component of the ParDE toxin-antitoxin systems. It is encoded downstream of two operonized tandem tniQ family genes (encoding a 314aa and 506aa TniQ and TnsD, respectively). The ParD component in ParDE systems binds the promoter region of the ParDE system on plasmids, suggesting it has target selector-like functionality. However, it is not predicted to interact with TnsC or the TniQ or TnsD proteins. The last candidate is highly spread (the most spread candidate) and hhsearch profile profile comparison with pfam highlight a match in its N-terminal region (hhprob=94.5; evalue=0.07; from positions ˜50-160) with pf18551.4 annotated as Thaumarchaeal output domain 1 and described as a metal binding domain found in prokaryotes. The candidate is also predicted to interact with TniQ predicted with AF2 multimer (Evans, R. et al. Protein complex prediction with AlphaFold-Multimer. bioRxiv (2021) doi: 10.1101/2021.10.04.463034; Mirdita, M. et al. ColabFold: making protein folding accessible to all. Nat. Methods 19, 679-682 (2022)). Considering the widespread of the latter candidate and its predicted direct interaction with TniQ, it was further investigated computational and experimentally. This new candidate was named TnsF as an alphabetical logic order after TnsA, TnsB, TnsC, TnsD.

### CAST I-D Analysis

For CAST I-D, 6 contigs encoding the system were extracted (FIG. 8A). Two of these loci are located near the edge of the contig and are incomplete. Three loci lack cas3d and harbor a dead Cas10d (the large subunit of the subtype I-D effector complex) in which the Histidine Aspartate (HD) nuclease has been inactivated (FIG. 12A).

### TnsF and Alternative Naming in Literature

Applicant named TnsF as a logical alphabetical order after TnsA, TnsB, TnsC, TnsD, TnsF. In Tn6022 transposons TnsA, TnsB, TnsC are sometimes denoted TniC, TniA, TniB (Partridge, S. R., Kwong, S. M., Firth, N. & Jensen, S. O. Mobile genetic elements associated with antimicrobial resistance. Clin. Microbiol. Rev. 31, (2018)), TniQ was referred as orf2 or TniD (Partridge, S. R., Kwong, S. M., Firth, N. & Jensen, S. O. Mobile genetic elements associated with antimicrobial resistance. Clin. Microbiol. Rev. 31, (2018); Petassi, M. T., Hsieh, S.-C. & Peters, J. E. Guide RNA categorization enables target site choice in Tn7-CRISPR-Cas transposons. Cell 183, 1757-1771.e18 (2020)), and TnsF as orf3 or til. Finally, tnsE which (FIG. 4) which is located near the left end, after the cargo was previously referred to as orf4 (Partridge, S. R., Kwong, S. M., Firth, N. & Jensen, S. O. Mobile genetic elements associated with antimicrobial resistance. Clin. Microbiol. Rev. 31, (2018); Petassi, M. T., Hsieh, S.-C. & Peters, J. E. Guide RNA categorization enables target site choice in Tn7-CRISPR-Cas transposons. Cell 183, 1757-1771.e18 (2020)).

### Mining of TnsF Relative

Applicant mined and visualized the diversity of the TnsF hits by drawing a graph network using hit BLAST comparisons (see Methods). Applicant found that TnsF forms a dense connected cluster but is also connected to another cluster (FIG. 13A) formed by larger proteins.

These larger proteins share a similar domain architecture with TnsF but retain the tyrosine recombinase catalytic site in the C-terminal region (CAT) (FIG. 13B). Applicant extracted 1099 non-redundant TnsF homologs including Tn6022 TnsF and these nuclease-active larger TnsFs.

### Characterization of CyCAST

The previously characterized CAST systems have been shown to mediate either bidirectional or unidirectional transposon insertion (Strecker, J. et al. RNA-guided DNA insertion with CRISPR-associated transposases. Science 365, 48-53 (2019); Klompe, S. E., Vo, P. L. H., Halpin-Healy, T. S. & Sternberg, S. H. Transposon-encoded CRISPR-Cas systems direct RNA-guided DNA integration. Nature 571, 219-225 (2019); Saito, M. et al. Dual modes of CRISPR-associated transposon homing. Cell 184, 2441-2453.e18 (2021)). To define the directionality of the CyCAST-mediated insertion, four pairs of primers were tested to recover each junction between target and insertion (FIG. 12C). Given that CyCAST likely requires a protospacer adjacent motif (PAM) to recognize target DNA, Applicant targeted a plasmid (pTarget) library containing protospacer adjacent motif 1 (PSP1) flanked by a 6N motif upstream of the protospacer with a single CyCAST protein expression vector (pHelper), as previously described (Strecker, J. et al. RNA-guided DNA insertion with CRISPR-associated transposases. Science 365, 48-53 (2019)).

### Prediction of the Insertion Site of Th6022 and Tsy

Applicant searched for contigs with a full comM gene and mapped the insertion sites of Tn6022 and this non-Tn7 putative system. Applicant found that both are inserted in the same region of comM 66 bp apart from each other but the directionality of insertion is different, suggesting different modes of insertion. The insertion of this putative mobile element into the same homing site as Tn6022 suggests that the TnsF and TnsF-like proteins perform similar target selection via a region within ZF, CB1, and CB2 domains that specifically recognizes the comM gene (FIG. 13D).

### Tsy Ends and Homology Arms

Applicant manually defined the ends of ZooTsy based on conservation at 135-bp for end1 (upstream the YRec to the border of comM) and 39-bp for end2 (end of cargo till the border of comM) and test 5 differents extensions (called homology arm and hereafter hom) for each end with comM sequence upstream end1 (hom1) and downstream end2 (hom2) to test if comM itself encode any motif for end recognition. YRec usually works as a dimer to recognize a region with two DNA motifs (each bound by the CB domain of each monomer) and cleave the middle region surrounding these motifs during recombination (Jayaram et al., 2015). In an excision scenario where the cleavage site for excision is at the edge of the partial comM gene, one motif should be located within comM gene while the other motif should be located downstream comM in the end of the transposon which would lead to a cleavage site at the transition area between comM gene and the end region.

### DISCUSSION

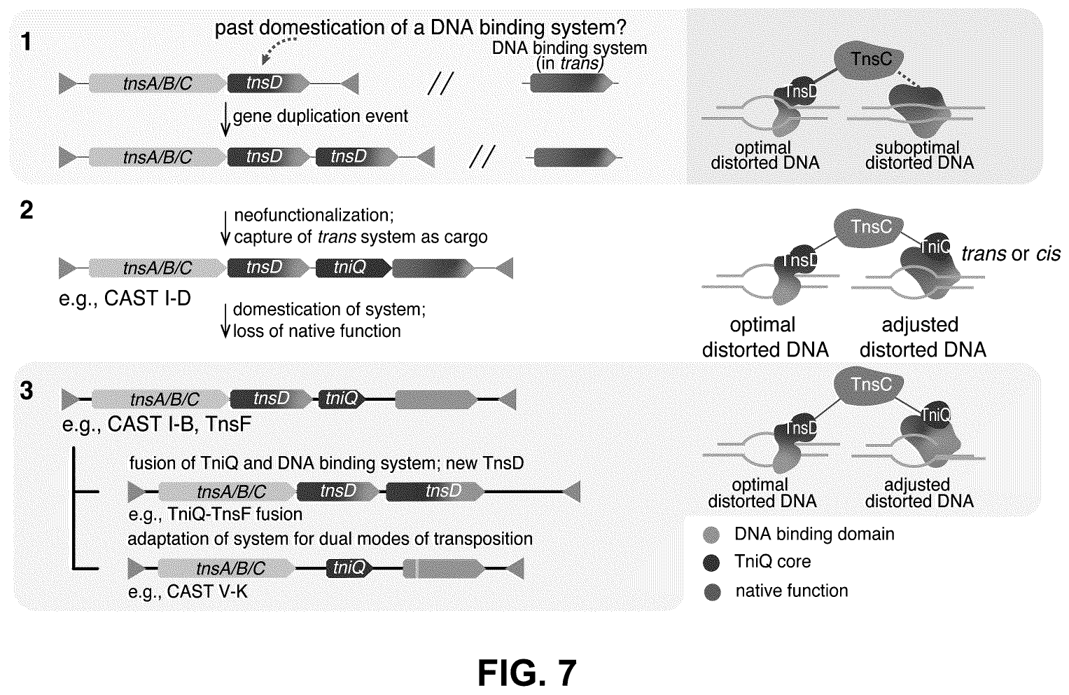

Target site selection is a crucial step in the life cycle of TEs because insertion of the element can dramatically affect the fitness of both the host and the TE depending on the insertion site (Durrant, M. G., Li, M. M., Siranosian, B. A., Montgomery, S. B. & Bhatt, A. S. A bioinformatic analysis of integrative mobile genetic elements highlights their role in bacterial adaptation. Cell Host Microbe 28, 767 (2020); Touchon, M., Bobay, L.-M. & Rocha, E. P. C. The chromosomal accommodation and domestication of mobile genetic elements. Curr. Opin. Microbiol. 22, 22-29 (2014)). Moreover, the choice of target site is critical for the TE to spread horizontally. It was recently reported that Tn7-like transposons co-opted CRISPR systems for RNA-guided target-site selection in addition to using protein-guided target selection. Here, Applicant expands on the understanding of target site selection mechanisms, identifying previously uncharacterized target site selectors and a novel TE. Together, these results indicate a modular architecture used by Tn7-like transposons to bridge target-site selection with transposase activity, providing TEs with maximal flexibility in targeting while maintaining conserved activity (FIG. 7).

This flexibility is manifested in the various proteins that Tn7-like transposons have co-opted and adapted for target-site selection. The results disclosed herein provide several examples of this. Although the CRISPR systems in most CASTs have lost their ancestral interference and nuclease functions, the CyCAST system was found to have an interference component and active Cas10d. Very recently, other CAST I-D systems were reported, but these have fully domesticated Cascade I-D whereby Cas3d is absent and HD nuclease of Cas10d is dead (Hsieh, S.-C. & Peters, J. E. Discovery and characterization of novel type I-D CRISPR-guided transposons identified among diverse Tn7-like elements in cyanobacteria. Nucleic Acids Res. (2022) doi: 10.1093/nar/gkac1216). Thus, CyCAST may be an evolutionary intermediate where the CRISPR system has been co-opted by the TE, but has not fully lost its native function. Similarly, Applicant found the Tn7-like transposon Tn6022 has co-opted TnsF, a catalytically inactive derivative of a tyrosine recombinase containing protein, whereas the Tsy transposon described here encodes a homolog of TnsF with an active catalytic domain. Applicant showed that TnsF from Tn6022 and Tsy interact with TniQ, the bridge to the transposase machinery. These results highlight the advantages of using TniQ as a hub to expand target-site selection by interacting with other sequence-specific DNA recognition partners, a process that may evolve into complete co-option of these partners, which then lose their original function(s).

In the case of Tsy, however, the nuclease function of TnsF is required for transposition (FIG. 121). Interestingly, Applicant also found that, in contrast to most transposons, Tsy does not have inverted repeats or conserved sequence patterns at its ends. Instead, Applicant found that it requires ends of different size (135 bp and 20 bp) and a 12-bp homology arm matching the insertion site upstream of end1. The requirement for homology arms, along with the finding that a catalytically dead TnsF does not support transposition, suggests an active role of TnsF (and/or Yrec, which is also encoded by Tsy) in excision whereby the recognition motifs surround the cleavage site, leaving half of the motif in the transposon after excision. The different lengths of the ends, however, suggest a distinct excision mechanism for each end. TnsF may also participate in the recognition of the ends through its DNA binding domain.

Applicant showed TnsF can target a heterologous comM gene, specifically the highly conserved Walker B motif, suggesting it may confer the ability on its host TE to spread across species. Although both Tn6022 and Tsy target this same attachment site, a unique characteristic of the Tsy system compared to Tn6022 is the lack of target duplication (TSD), which is consistent with previous findings that tyrosine recombinase driven MGEs do not create TSDs (Smyshlyaev, G., Bateman, A. & Barabas, O. Sequence analysis of tyrosine recombinases allows annotation of mobile genetic elements in prokaryotic genomes. Mol. Syst. Biol. 17, e9880 (2021)). This raises the possibility that, although insertion by Tn6022 or Tsy will impair comM, excision of Tsy could restore comM integrity, and the encoded ComM might play a role in the spread of Tsy.

Applicant's finding that other enzymes have been co-opted by Tn7-like transposons raises the question of how new target selectors originate. In Tn7, to recruit the transposase machinery to the target site, TnsD binds to and induces a local distortion of the DNA, which attracts TnsC. Artificial induction of such DNA distortion has been shown to attract TnsC independently of TnsD (Kuduvalli, P. N., Rao, J. E. & Craig, N. L. Target DNA structure plays a critical role in Tn7 transposition. EMBO J. 20, 924-932 (2001); Rao, J. E., Miller, P. S. & Craig, N. L. Recognition of triple-helical DNA structures by transposon Tn7. Proc. Natl. Acad. Sci. U.S.A. 97, 3936-3941 (2000)). Native Cas effectors (Jiang, F. & Doudna, J. A. The structural biology of CRISPR-Cas systems. Curr. Opin. Struct. Biol. 30, 100-111 (2015)) and TnsF likely induce similar distortions in the target DNA (i.e., R-loop formation by Cas effectors and DNA bending by tyrosine recombinase-containing proteins including TnsF). Although a canonical tyrosine recombinase would entirely cover the distorted DNA, precluding TnsC access, the distinct architecture of TnsF, with two different DNA binding domains, may bend the DNA while still allowing partial or full access for TnsC. Thus, proteins that provide more efficient target selection may supersede the target selecting role of the C-terminal region of TnsD, ultimately leading to their domestication and loss of their native enzymatic activity (FIG. 7). CAST-I-D and the TnsF homolog in the Tsy system are examples of intermediate stages on the evolutionary path to domestication. Applicant also detected numerous loci encoding a TniQ that is too short to enable target selection and no other identifiable target selector partner. Such elements might recruit target selectors in trans, perhaps representing the very first step in the generation of a new target selector or even greater flexibility in target selector partnering.

### Methods

### Identification of Th7-Like Transposons

HMMs of Tn7 proteins, i.e., TnsA (PF08721.12, PF08722.12), TnsB (PF00665.27), TnsC (PF05621.12, PF11426.9, PF13401.7), TnsD PF06527.12, PF15978.6), and TnsE (PF18623.2) were used to search homologs with hmmsearch software (Eddy, S. R. Accelerated profile HMM searches. PLoS Comput. Biol. 7, e1002195 (2011)) (using option ga_cut) within predicted protein sequences derived from microbial contigs available in the NCBI Genbank and WGS databases, JGI database (projects with stated permission to use), and the MG-RAST database (Meyer, F. et al. The metagenomics RAST server-a public resource for the automatic phylogenetic and functional analysis of metagenomes. BMC Bioinformatics 9, 386 (2008)) (all frozen in November 2020). The full database encompasses 521,828,662 contigs in total (contigs greater than 1.5 kb) covering 6,932,321,054,498 bp of genomic DNA. 1,617,895 contigs have detectable rpob genes (detected with hmmsearch from TIRGR02013.1 profile) suggesting the diversity is probably reflected by 1.6 million genomes (Vos, M., Quince, C., Pijl, A. S., de Hollander, M. & Kowalchuk, G. A. A comparison of rpoB and 16S rRNA as markers in pyrosequencing studies of bacterial diversity. PLoS One 7, e30600 (2012); Ogier, J.-C., Pagès, S., Galan, M., Barret, M. & Gaudriault, S. rpoB, a promising marker for analyzing the diversity of bacterial communities by amplicon sequencing. BMC Microbiol. 19, 171 (2019)). Loci were built by mapping the location of the hits into the contig and aggregating hits when they are no further than 20 kb from each other. Loci were selected if they satisfied the following criteria: (i) at least 2 hit genes homologous to 2 distinct Tn7 components, (ii) 2 of the hit genes are in a putative operon, which is operationally defined as 2 codirectional ORFs separated by less than 50 bp of non-coding sequence, and (iii) the hit genes are less than 3 kb from the contig boundary (to remove likely incomplete transposons). Using these criteria, 80,028 loci were obtained from which 86,517 TnsC homologs were extracted and translated. These homologs were clustered at 80% sequence identity over 75% of the protein length (coverage) using MMSeqs2 (v. 12-113e3) (Steinegger, M. & Söding, J. Clustering huge protein sequence sets in linear time. Nat. Commun. 9, (2018)) and obtained 7,789 TnsC homolog representatives.

### Construction of the Phylogenetic Tree of TnsC Homologs

The protein sequences of representative TnsC homologs were aligned using a method described previously (Wolf, Y. I. et al. Origins and evolution of the global RNA virome. MBio 9, (2018)). Briefly, the protein sequences were clustered at 50% identity and cluster members were extracted and aligned using MUSCLE (version 5) (Edgar, R. C. et al. Petabase-scale sequence alignment catalyses viral discovery. bioRxiv (2020) doi: 10.1101/2020.08.07.241729). An all-versus-all comparison of the multiple sequence alignments (MSAs) was computed using HHsearch (Steinegger, M. et al. HH-suite3 for fast remote homology detection and deep protein annotation. BMC Bioinformatics 20, 473 (2019)). An unweighted pair group method with arithmetic mean (UPGMA) dendrogram was constructed using the HHSearch similarity scores. The dendrogram was used to guide the iterative pairwise alignment of cluster MSAs using HHalign (Steinegger, M. et al. HH-suite3 for fast remote homology detection and deep protein annotation. BMC Bioinformatics 20, 473 (2019)). Clusters were discarded if they could not be aligned using this approach, leaving 6,988 sequences in the single final alignment. The alignment was first filtered to remove sites with conserved gaps using trimal version 1.2 with the option gappyout (Capella-Gutiérrez, S., Silla-Martínez, J. M. & Gabaldón, T. trimAl: a tool for automated alignment trimming in large-scale phylogenetic analyses. Bioinformatics 25, 1972-1973 (2009)). Finally, aligned sequences from which Walkers A and B were not aligned correctly (any gap in one of the positions) were discarded. This led to an alignment of 6,384 sequences that are used for this study. The alignment was input into FastTree2 (Price, M. N., Dehal, P. S. & Arkin, A. P. FastTree 2-approximately maximum-likelihood trees for large alignments. PLoS One 5, e9490 (2010)) with the Whelan-Goldman models of amino acid evolution and gamma-distributed site rates. The tree was visualized and annotated using the interactive tree of life (itoD (Letunic, I. & Bork, P. Interactive Tree Of Life (iTOL) v5: an online tool for phylogenetic tree display and annotation. Nucleic Acids Res. 49, W293-W296 (2021)).

### Annotation of the Genomic Vicinity of TnsC Homologs

TnsC homolog representatives were mapped onto their genomic contigs and genes within 50 kb distance were extracted. These genes were translated and annotated for specific genes of interest including Cas effectors (Cascade and RAMP components and single protein effector from Class 2 CRISPR-Cas systems) and Cas6 that were annotated using the profiles extracted from DefenseFinder (Tesson, F. et al. Systematic and quantitative view of the antiviral arsenal of prokaryotes. Nat. Commun. 13, 2561 (2022)) and hmmsearch (Eddy, S. R. Accelerated profile HMM searches. PLoS Comput. Biol. 7, e1002195 (2011)) with a hit threshold set at a score of 25 whereby all hits with a score of 25 or greater were selected. Hits were mapped onto leaves of the TnsC homolog tree and subsequently the TniQ homolog tree and displayed with itol (FIGS. 2 and 9B). As TniQ/TnsD can be extremely divergent and therefore not always detected by hmmsearch, a profile-profile comparison was performed using HHalign software (Steinegger, M. et al. HH-suite3 for fast remote homology detection and deep protein annotation. BMC Bioinformatics 20, 473 (2019); Söding, J., Biegert, A. & Lupas, A. N. The HHpred interactive server for protein homology detection and structure prediction. Nucleic Acids Res. 33, W244-8 (2005)) and the TniQ PF06527 profile to annotate remote TniQ homologs. TniQ hits were selected if they have a hhsearch probability >=80. Given the abundance of TniQ, false positive detection of TnsC (AAA-ATpase among cargo), and sometimes multiple Tn7 transposons co-occurring, it is challenging to know which tniQ/tnsD is associated with which tns C. For a given tnsC, Applicant defined an association with tniQ/tnsD (s) if no other tns C homologs were found in the vicinity. If there was a tns C homolog closer than the given tnsC, Applicant associated tniQ/tnsD to it only if there was a tnsB gene operonized with this closer tns C. In total 5,072 tniQ/tnsD were found to be associated with TnsC homolog representatives. The presence of these tniQ/tnsD were mapped onto the TnsC homolog tree. When multiple tniQ/tnsD were found in the vicinity, the two largest were selected and the second selected tniQ/tnsD were mapped on an additional barplot indicating the size of the second protein in aa. tnsE were detected in relatives of E. coli Tn7 but also weakly in Tn6022-like transposons where it is encoded in the far vicinity of the Tn core components (in contrast to E. coli Tn7 relatives where tnsE is part of a full operon encompassing all Tn core components and tnsD). Manual inspection indicated these TnsE remote homologs are encoded near the left end of the transposon far from the other transposon components. Structural comparison from the models predicted by Alphafold2 (Jumper, J. et al. Highly accurate protein structure prediction with AlphaFold. Nature 596, 583-589 (2021)) (FIG. 10A-10B) confirmed that these hits are TnsE relatives. These remote TnsE were used as a new seed to annotate more TnsE using blastp on all translated genes in the vicinity of tns C. 552 TnsE in total were detected, and their presence were mapped on the TnsC homolog tree and on the TniQ homolog tree and displayed using itol framework (Letunic, I. & Bork, P. Interactive Tree Of Life (iTOL) v5: an online tool for phylogenetic tree display and annotation. Nucleic Acids Res. 49, W293-W296 (2021)).

### Structural Prediction and Analysis

All structural models were built using Alphafold2 (AF2) software under the colabfold framework installed locally (Evans, R. et al. Protein complex prediction with AlphaFold-Multimer. bioRxiv (2021) doi: 10.1101/2021.10.04.463034; Jumper, J. et al. Highly accurate protein structure prediction with AlphaFold. Nature 596, 583-589 (2021); Mirdita, M. et al. ColabFold: making protein folding accessible to all. Nat. Methods 19, 679-682 (2022)). Multiple sequence alignments were constructed using colabfold_search on colabfold database that includes Uniref and environmental protein sequences. Alignments were input into AF2 and generated 3 models with 35 recycles. All models were examined using the PyMOL framework (The PyMOL Molecular Graphic System Version 1.2, Schrodinger, LLC) mapping the predicted local distance difference test (plDDT) (Jumper, J. et al. Highly accurate protein structure prediction with AlphaFold. Nature 596, 583-589 (2021); Mariani, V., Biasini, M., Barbato, A. & Schwede, T. IDDT: a local superposition-free score for comparing protein structures and models using distance difference tests. Bioinformatics 29, 2722-2728 (2013)), a local measure of prediction confidence, on each residue. Regions of the proteins with plDDT inferior to 50 were not considered. Protein docking prediction was performed using AF2 with the multimeric model (Evans, R. et al. Protein complex prediction with AlphaFold-Multimer. bioRxiv (2021) doi: 10.1101/2021.10.04.463034). Results of protein docking were analyzed by examining the predicted aligned error (PAE) matrix (Jumper, J. et al. Highly accurate protein structure prediction with AlphaFold. Nature 596, 583-589 (2021)) and visualizing the interaction area on PyMOL. The spatial distribution of specific chemical interactions found in protein-protein interactions (Andreani, J., Faure, G. & Guerois, R. Versatility and invariance in the evolution of homologous heteromeric interfaces. PLoS Comput. Biol. 8, e1002677 (2012); Faure, G., Andreani, J. & Guerois, R. InterEvol database: exploring the structure and evolution of protein complex interfaces. Nucleic Acids Res. 40, D847-56 (2012)) were analyzed using PyMOL to validate models when PAE was weak. The predicted structures with high confidence (typically average pLDDT>70) were considered for downstream analysis. Search for structural similarity was performed using DALI software version 226 using the PDB50 (non-redundant at 50% of sequence identity) and a custom made database made from the EBI Alphafold2 database. This database contains Alphafold2 models of Uniref50 (Steinegger, M. & Söding, J. Clustering huge protein sequence sets in linear time. Nat. Commun. 9, (2018); Suzek, B. E., Huang, H., McGarvey, P., Mazumder, R. & Wu, C. H. UniRef: comprehensive and non-redundant UniProt reference clusters. Bioinformatics 23, 1282-1288 (2007)) extracted from EBI Alphafold2 database (https://alphafold.ebi.ac.uk/) restricted to models having at least 30aa with a plDDT greater than 50 (hereafter called AF2DB50). Hits with Z-scores greater than 5 were retained, and every hit was manually verified by building structural alignment in PyMOL editing mode.

### Phylogenetic Analysis of TniQ

5,072 tniQ/tnsD detected in the vicinity of the Tn7 tns C homolog representatives were translated and aligned using muscle version 5 (Edgar, R. C. et al. Petabase-scale sequence alignment catalyses viral discovery. bioRxiv (2020) doi: 10.1101/2020.08.07.241729) using the option super5 that clusters the sequences. Alignments were then created for each cluster and merged into a single alignment. The alignment was first filtered to remove sites with conserved gaps using trimal version 1.2 with the option gappyout (Capella-Gutiérrez, S., Silla-Martinez, J. M. & Gabaldón, T. trimAl: a tool for automated alignment trimming in large-scale phylogenetic analyses. Bioinformatics 25, 1972-1973 (2009)). As TniQ/TnsD can harbor very divergent C-terminal regions in terms of both sequence and size, the alignment was restricted to the core region of TniQ. To determine the core (roughly corresponding to the first 300 aa), the structure of all dual TniQ-TnsD CAST systems was predicted with AF2 and manually aligned structurally under the PyMOL framework. Core positions were mapped to the sequence alignment and the downstream regions (C-terminal region) were trimmed out. Finally, to filter out misaligned TniQ cores, aligned sequences from which the first CxxC motif of the zing finger were not well aligned (any gap in one of the positions) were discarded. The 4,916 remaining aligned sequences were used to build a tree using FastTree2 (Price, M. N., Dehal, P. S. & Arkin, A. P. FastTree 2--approximately maximum-likelihood trees for large alignments. PLoS One 5, e9490 (2010)) with the Whelan-Goldman models of amino acid evolution and gamma-distributed site rates. The presence of cas effector genes and cas6 as well as tnsE in the vicinity was shown similarly as for the TnsC tree. Operonized genes with tniQ/tnsD (see TniQ partner candidate analysis) were also displayed (light green ring). To investigate the origin of dual TniQ-TnsD, a visual approach and a statistical approach were used. A connector between dual TniQ-TnsD leaves of the tree was drawn and colored based on a rainbow gradient spread across all leaves from the left to the right of the tree. The connector has a uniform color picked from the color assigned to the left-most leaf. If a connector has a color that matched the color both at the starting leaf (left) and the arriving leaf (right), the proteins are closely related. Conversely, any contrast between the colors indicates the proteins are not closely related. For the statistical approach, the branch distances of all dual TniQ-TnsD were extracted and compared with 1000 random branch distances involving nondual TniQ-TnsD across the tree. Branch distances were calculated using the Phylo package from the Biopython library. Biopython package (Cock et al., 2009). Comparison of these distances was done via a T-Test using SciPy python library version 1.0 (Virtanen et al., 2020).

### Analysis of Candidate Partners of TniQ

Genes operonized with tniQ/tnsD were extracted and translated if they were not related to any Tn7 components (TnsA, TnsB, TnsC, TniQ/TnsD, or TnsE). 782 proteins were clustered at 30% of sequence identities and 30% sequences coverages using MMSeqs2 (v. 12-113e3) (Steinegger, M. & Söding, J. Clustering huge protein sequence sets in linear time. Nat. Commun. 9, (2018)). From each cluster, members were aligned together using mafft-linsi (Katoh and Standley, 2013). From each alignment, secondary structures were predicted psipred version 2.6 (Jones, 1999) to ensure compatibility with hhpred, and HMM profiles were built for each using hhmake (Söding et al., 2005). Each hhpred profile was compared to the Pfam protein domain database (preformatted for hhpred and available at https://wwwuser.gwdg.de/˜compbiol/data/hhsuite/databases/hhsuite_dbs) using hhsearch. Hits with hhsearch probability >=90 were considered. Candidates operonized with tniQ/tnsD were mapped onto the tree (light green ring). The 6 largest groups of candidates were selected based on the conservation of the operonized genes across several adjacent leaves in the TniQ tree and analyzed further. For each candidate, profile analysis was performed (using hhpred webserver (Söding, J., Biegert, A. & Lupas, A. N. The HHpred interactive server for protein homology detection and structure prediction. Nucleic Acids Res. 33, W244-8 (2005)) to assess their potential function and structural docking with TnsC and TniQ/TnsD to test for potential interactions (interaction with TnsC could highlight a novel target selector independent from TniQ/TnsD, whereas interaction with TniQ/TnsD could suggest a partner target selector) using Alphafold multimer.

### TnsF Analysis

TnsF from Acinetobacter johnsonii Tn6022 (hereafter AjTn6022 and AjTnsF) was chosen as the representative for computational and experimental analysis. HHpred was used to annotate the domain architecture region of AjTnsF and detect the presence of multiple zinc fingers in the N-terminal region (positions 1-180 has a hit to a LIM Zinc-binding domain-containing protein hhprob=97.23) and similarity with tyrosine recombinase in the C-terminal region (positions 328-493 has a hit to a site-specific recombinase IntI4 hhprob=99.14). A structural model of TnsF was obtained using AF2 and split into 3 domains defined by long linkers connecting globular regions and a long N-terminal region encoding several zinc fingers. Each domain was used as a seed for structural similarity search using DALI software across the PDB50 (as described above). Top hits were inspected manually with PyMOL.

### Mining of TnsF and Phylogenetic Analysis of Tnsf Homologs

To search for TnsF relatives, TnsF from Acinetobacter johnsonii Tn6022 (hereafter AjTn6022) Tn6022 was used as a seed for a psiblast (Altschul et al., 1997) search for 3 iterations on the NCBI NR database (in August 2022). 22,419 protein hits were extracted and clustered at 80% of sequence identity and 70% of coverage with MMSeqs2 (v. 12-113e3) (Steinegger, M. & Söding, J. Clustering huge protein sequence sets in linear time. Nat. Commun. 9, (2018)). A blastall comparison (Altschul and Lipman, 1990) was performed, and e-values associated with each comparison were input into CLANS software (Frickey and Lupas, 2004) to cluster hits according to their e-values and draw a graph network representation. Several clusters (point density connected and close to each other) are also connected to each other. AjTn6022 TnsF was mapped onto the graph to identify the cluster to which it belongs in order to define the Tn6022 TnsF cluster. The Tn6022 TnsF cluster is connected to another cluster from which several members were extracted and mapped onto genomic contigs. Genomic comparison between these contigs and Tn6022 contigs reveals a distinct system with partial comM surrounding the system and with no apparent Tn7 components but other genes operonized with tnsl. Hhpred webserver (Söding, J., Biegert, A. & Lupas, A. N. The HHpred interactive server for protein homology detection and structure prediction. Nucleic Acids Res. 33, W244-8 (2005)) was used to annotate these genes, revealing the presence of a gene encoding a tyrosine recombinase (yrec), a gene encoding a helix turn helix domain (hth), and a gene encoding a GIY-YIG nuclease. The system was named transposon using Target Selector based on tYrosine recombinase (Tsy) based on the components operonized with tnsl. Hits belonging to the CLANS Tn6022 TnsF cluster and the adjacent cluster harboring the Tsy TnsF were extracted and aligned using muscle version 5 (Edgar, R. C. et al. Petabase-scale sequence alignment catalyses viral discovery. bioRxiv (2020) doi: 10.1101/2020.08.07.241729) with the super5 algorithm. The alignment of 1,095 protein sequences was further trimmed using trimal version 1.2 (gappyout option) (Capella-Gutiérrez, S., Silla-Martinez, J. M. & Gabaldón, T. trimAl: a tool for automated alignment trimming in large-scale phylogenetic analyses. Bioinformatics 25, 1972-1973 (2009)) and was input to FastTree2 (Price, M. N., Dehal, P. S. & Arkin, A. P. FastTree 2--approximately maximum-likelihood trees for large alignments. PLoS One 5, e9490 (2010)) with the Whelan-Goldman models of amino acid evolution and gamma-distributed site rates. The tree was visualized and annotated using the interactive tree of life (itoD (Letunic, I. & Bork, P. Interactive Tree Of Life (iTOL) v5: an online tool for phylogenetic tree display and annotation. Nucleic Acids Res. 49, W293-W296 (2021)). Ins genes were mapped to genomic contigs, and genes in the vicinity (20 kb) were extracted, translated, and further clustered at 30% of sequence identity retaining 50% of coverage using MMSeqs2 (v. 12-113e3) (Steinegger, M. & Söding, J. Clustering huge protein sequence sets in linear time. Nat. Commun. 9, (2018)) and each cluster was converted into HMM profile using hhmake and compared to the profile pfam database using hhsearch (Söding, J., Biegert, A. & Lupas, A. N. The HHpred interactive server for protein homology detection and structure prediction. Nucleic Acids Res. 33, W244-8 (2005)). The top populated clusters were Tn7 components (tnsA, tnsB, tns C, and tniQ/tnsD and candidates operonized with Tsy TnsF (yrec, GIY-YIG). The presence of tniQ thsD as a marker of Tn7, and the presence of the yrec and the GIY-YIG nuclease were mapped on the TnsF tree as distinct rings. Split comM genes were extracted and translated from AjTn6022 and one Tsy locus and used as seeds to search for the full protein version of Mg chelatase for Tn6022 and Tsy using blastp (Altschul and Lipman, 1990). The closest Mg chelatase was selected for each of the two systems and used as a seed to detect comM pieces in the nucleotide vicinity of each locus using tblastn (Altschul and Lipman, 1990). The presence of a comM hit is indicated as a ring on the TnsF tree. Inspection of the tree shows Tn6022 TnsF is monophyletic (branch support=0.976). A TnsF from Tsy was extracted from Zoogloea sp. LCSB751 (ZooTnsF) and the structure was predicted using AF2. Structural comparison between AjTnsF and ZooTnsF was performed manually using a PyMOL framework. The structural similarity search was done using DALI on AF2DB50 (as described above). Hits with a zscore greater than 8 were inspected manually to search for tandem CB+CAT domain architecture using PyMOL.

### Prediction of Transposon Ends for Th6022 and Tsy

Transposon ends for AjTn6022 were determined using Geneious searching for at least one distinct cluster of short repeats (12 nt with 3 mismatches maximum, repeated at least twice in each end) that surround the transposon components including the tsA, tnsB, tnsC, tniQ, tnsF, and tnsE. Exact end boundaries were then adjusted manually based on local alignment of the clustered repeats area and search for target site duplications.

Nucleotide sequences for the right and left transposon ends of CyCAST, AjTn6022, and ZooTsy can be found in Table 6, below.

TABLE 6

| System Type | RE (5′ to 3′) | LE (5′ to 3′) |
|---|---|---|
| CyCAST | Tgtgactgcaaccataagttgtccgatttt | Tgtgactgcaaccataagttgtctatttcgga |
| 40005\|0\|GCA_ | gaaagcataagttgtccactcatgggtgtg | cactttatgcttacaattttggctggacagctta |
| 000022045.1_ | gacaacttgagtcatcgtcaatattcacttt | tgcttacaattttggctggacagatagagcga |
| ASM2204v1_ | gcaaaataaaaaactgaaagcataagttg | aagcaataggcaagacgattatatttctttgcc |
| genomic\| | cccaaatgttaacaatcgaaagcataagtt | tataaagtaggcctattgatggatgaccctcc |
| CP001344.1\| | gtccaaaagttagaacttggagcagccgt | aac (SEQ ID NO: 85) |
| 5374574\| | tgtgttgccttagggttatttgccaagagca | |
| Cyanothece | agtatgcttttaatcctggaaaagctccga | |
| | aggaatgacttct (SEQ ID NO: | |
| | 84) | |
| AjTn6022 | Tgtcatttacagcaatagataagagtgtca | Tgtcagttccacaaataaatcagagtttacac |
| ENU40864.1 | tgataagaaaacaataggttgccactctaa | aaataaactagagtgataataaaaactatact |
| (Acinetobacter | aattacacaaatagattagagtaatgacag | atttatatatgaatttcattaaattcactaaaaga |
| johnsonii | taataagcaagagtggcttgcaaatttaca | tatgcctgcaaataaaaaagttaaagtcacaa |
| CIP 64.6 | caaatacgcacgagtggctgtaagaggc | ctattgattgtttcccaaatgataatattcgcag |
| acLZl- | attct (SEQ ID NO: 86) | aactgtctatagatatggtgggtttcttaggaa |
| supercont1.5.C9, | | tgagggcagtga (SEQ ID NO: 87) |
| whole genome | | |
| shotgun | | |
| sequence) | | |
| ZooTsy | Aagttctgatttagccaatgtagcaacga | Ggactgggattctccaatat (SEQ ID |
| Zoogloea sp. | acaaggcaaaaattcggataacccactga | NO: 89) |
| LCSB751 NODE_ | ctataaaggaatcccaaagtatccaatgtc | |
| 6_length_ | gcattacttgcaggaatcctgtgagtgatg | |
| 275285_cov_ | tcacagcattagaagta (SEQ ID | |
| 54.3012_ID_11, | NO: 88) | |
| whole genome | | |
| shotgun | | |
| sequence | | |

Computational analysis of CAST I-D

A blastp search was performed from CAST I-D Cas10d on the NCBI NR database to fetch additional loci. Only one candidate, from the Cyanothece sp. PCC 7425 genome (CyCAST), harbors a predicted active HD and Cas3′. The CRISPR-array of CyCast was predicted using minced (Bland et al., 2007) with default parameters, and transposon ends were determined as described above. Exact end boundaries were then adjusted manually based on local alignment of the clustered repeats area and search for target site duplication. CAST I-D Cas10d was used as a seed to search for homologs using blastp on the NCBI NR database. An initial phylogenetic tree (using FastTree) was done from the sequence alignment (using mafft-linsi) of the hits, and the subtree harboring all CAST I-D Cas10d and a few non-CAST I-D Cas10d were extracted. A structural model of CyCAST Cas10d was obtained using AF2 and used to determine the position of the catalytic sites of the HD nuclease domain. These positions were mapped onto the alignment from which a sub-alignment encompassing these positions and their local sequential vicinity was built and juxtaposed next to the tree.

### Plasmid Construction

All plasmids used in this study are described in Table 7. For type I-D CyCAST experiments, genes encoding Cyanothece sp. PCC 7425 (ASM2204v1_genomic|CP001344.1|5374574|) TnsAB-TnsC-TniQ-TnsD, and Cas10d-Csc2-Cas5-Cas6 were synthesized (Twist Bioscience) and cloned into pCDFDuet-1 with the appropriate spacer flanked by two CRISPR direct repeats, yielding pHelper (CyCAST). Gene fragments encoding both transposon ends (Genewiz) were cloned into pBluescript II SK (+) (Agilent), yielding pDonor (CyCAST). For PAM screens, a 0.5-kb exon fragment amplified from human EMX1 was inserted between the transposon ends as a mock non-functional cargo in E. coli. For AjTn6022 experiments, genes encoding Acinetobacter johnsonii Tn6022 locus TnsA-TnsB-TnsC-TnsD-TnsF weresynthesized (Twist Bioscience) and cloned into pCDFDuet-1. Predicted AjTn6022 transposon ends (RE and LE) were synthesized (Genewiz) and cloned into pBluescript II SK (+) with a kanamycin resistance gene to obtain pDonor (AjTn6022). For ZooTsy experiments, genes encoding Zoogloea sp. LCSB751 Tsy locus YRec-HTH-TnsF-GIY-YIG nuclease were synthesized (Twist Bioscience) and cloned into pCDFDuet-1. Predicted ZooTsy transposon ends (endl and end2) were synthesized (Genewiz) and cloned to obtain pDonor (ZooTsy). Through the study, pBluescropt-donor was used for CyCAST, and pR6K-donor (backbone: addgene #127924) was used for AjTn6022 and ZooTsy experiments, except Tagmentation-based Tag Integration Site Sequencing (TTISS) analysis (Schmid-Burgk, J. L. et al. Highly Parallel Profiling of Cas9 Variant Specificity. Mol. Cell 78, 794-800.e8 (2020)) with pSC101-donor (backbone: addgene #140630). To construct pHelper variants for CyCAST, AjTn6022, and ZooTsy, the Q5 Site-Directed Mutagenesis Kit (NEB) was used. For pHelper (AjTn6022) TniQ point mutants, a ribosome binding site and a partial TnsF N-terminal coding sequence was inserted to separate overlapped ORFs of TniQ and TnsF, and mutations were introduced into TniQ. The tRNA-Val gene of pTarget (PmcCAST) (addgene #168163) was replaced by either tRNA-Leu gene of Cyanothece, comM gene fragment of A. johnsonii, or comM gene fragment of Zoogloea sp. LCSB751, yielding pTarget (CyCAST), pTarget (AjTn6022), or pTarget (ZooTsy), respectively. For TniQ and TnsF proteins purification, TniQ of AvCAST, PmcCAST, CyCAST, Tn7017 and AjTn6022 were individually cloned into ColE1-based pTwinStrep-SUMO bacterial expression vector, and ShCAST (addgene #135527) were used. TnsF of AjTn6022 and nuclease-dead TnsF-Y584F mutant of ZooTsy were individually cloned into ColE1-based pTwinStrep-bdSUMO bacterial expression vector.

TABLE 7

| Plasmid description | Plasmid DNA sequence |
|---|---|
| pHelper(CyCAST)_PSP | ggggaattgtgagcggataacaattcccctgtagaaataattttgtttaactttaataaggagatat |
| 1 | accatggtttccaagcctgaatcccggtctacgggactgaaacgagaagtcatttaataaggcc |
| | actgttaaacgtaggtttccaagcctgaatcccggtctacgggactgaaacgatccgaattcga |
| | gctcggcgcgcctgcaggtcgacaagcttgcggccgcataatgcttaagtcgaacagaaagta |
| | atcgtattgtacacggccgcataatcgaaattaatacgactcactataggggaattgtgagcgga |
| | taacaattccccatcttagtatattagttaagtataagaaggagatatacatatggctaccctactcc |
| | aaaccctcctgttacgaacgttgtcacccgaggcggatccagttctctatgtctacattgagacg |
| | gtactacctgcaatggaacgggaatttggcttgattccagcccttggtggctcggaggaagcac |
| | actttcatctgctgaggcgacggggaaacaaatatgcggcggaaaattccaagcggtacgcca |
| | gtcgctctgatcaaagcctgctggttcatgtgctcaatgccttgctgaccgcctggaatttgttgc |
| | cattcctaagctccacatttgaactatcagatgatgagaaacggctgctctgcctggggctgacc |
| | ctacacgattacaacaagtggtgccagggtgaggaggaagattcgccgattgcatgcgaagta |
| | gcagagattcttgagctttgtcaaacattgggagagcggttgaactttgacggattctgggcaga |
| | gtggcgggagtatttgacagagattgcctaccttgcccaaaacacccagggaaaagttggaac |
| | aaatctacccaaagcgaattggggcagcttcaaaattctagatacccgtcgtctggattcgcccc |
| | tacgatggctactcgcttttggcgatgtcgcagtccatctcagcgatccatctgacatcgcaatga |
| | aaacggggggagatcgcctgcgggaacatctgcgctttctaggtattcgtcgcaagctggtcta |
| | ccatcgcctgcgcaactgtacagggctattaaccaacggaattcataatgcggtaatgcattttg |
| | ctagagaactggattggcaacccatcctcttctttgctcagggtgttgtttatctcgctcctcaggg |
| | tgctggcgttccggatcgcttagaacttcaggaatttctctgggagcaggtcagcaaccgattag |
| | ccaccagcatgttgcggggagaggttggtttcaaacgggatggcaagggactcaaggtggct |
| | ccccaaaccctggaactgttctcccctgctcagttgattcggaacttgcccggtgttgtccaatctt |
| | atgtcggaaacgctaaatctcctgcgactccaaaacgacttgagaagctggaactcagttcaac |
| | agagcgagagtttttggctaaaggtgcagatttaagagcagatcgcattgccgagtttatctttct |
| | ggcgcaaaaagagtttttcggagataaaccagaatttatcacttggatgcttgaaactctggggg |
| | ttcaaactgctatcacttcggagcagacgcagcttcaatccggtggtgtgaactggggatggtat |
| | cacgcagcagcctattacgtcgcgagcaatgccacgctggatttggaagagatttcggagaaa |
| | cttcaggaatttgccgagcagttggcaacctgggcggaggaaaacaatttcttcccagagcatc |
| | gcagcccaacccatgacatctttttcaactatctgactcaatacctggaagttcaggggtgggag |
| | cagcaggagcagtcctttcagcttgaactggcaggctatgttggcgcaaagactaaaactgcca |
| | gacaaccgatttgctccctcagttccggtgaatttgcttcggaagaccagatggattctgttgtcct |
| | gttcaaaccgcagcagtacagcaacaagaatcctctcggtggacggcagatcaagcggggg |
| | atttccaaaatctggtccctggaaatgctgctcaggcaggctctttggtctgcgccagcaggcaa |
| | actagaggaacaacaacccattttcctttacatcttcccggcatacgtttactcgccccaggttgct |
| | gctgctgtccggttactggtgaatgatctgaagcgtgtcaacctctgggaagtccgtaaacactg |
| | gttggagaatggcatggatgctcaggcgttgcgatcgtttccctggctagtcggtgacaaaaac |
| | gattcgggtaactctgctccatccagatactctggtgaagacttgcctttcatggcaatgaactac |
| | accaccaccagaggcaaaacgataactgatgcctgggttgaacctgctttcctgacactggtgc |
| | tgcctttgttgctgggtgtgaaagttgtcgccaccagtagccagattccactctatgaaagcgac |
| | aacgacttttgcgaatctgtcaagctggatggacccgccgatttctgggctttgctgcggctacc |
| | aacttccctgcatctccaggattggattggggggcgtgttcaaaatttagcagaaggcttgaacc |
| | gcttgatggttgcctactgcctgcatctggacagtcgtagcgctccaccagatgcgcgctggag |
| | agccttaaacggcagtgtccgagagatgacaacaaatgttctaaacattttttcacttgcagagg |
| | agggattacgtcgtgatggctctaaacgcgatgcaacagttgaagaaattaaccgctattggaa |
| | atacgcccagctgtgggcagagggagactctgatatgcaaaacaagttgaagattaccaaacg |
| | cttagtcgatgagtaccgccagttttacagagccagtgcgaacgaatccagccatacggttttgc |
| | tgccattttctaaggcactggaacacattctttcggtaccaaatgattgggacgatgaggaaatta |
| | ttgttcaaggggcaggacagttacaggccgcgttagatcggcaggaagtctacaaacgtcccc |
| | tcatgcttgacaaagagattccctttgaaattcgtcggatgaaggagtttgaggcaattcaaacct |
| | tcatgaccacctgcgtcattgacctatttggacaaatgtgtaaaggcgatcgcgcgctcctgcag |
| | gaacatcgcaaccgccttaaatcaggggtcgaatttgcctatcgtcagttagccctgcaagaga |
| | aacaagccaaagccaaccaaaaatctggagaacaatcatgagcattcttgaaaccttaaagcc |
| | agagttccacacagccttccctcgcctcgcctctggcaaatatgtccatttcttcatggtgcgtca |
| | cagccagtcgttccccgtctttcaaaccgatggcgttttgaacactgcccgtactcaggctgggtt |
| | aaatgcaggcgatgaaaaatccagacaggcaatcagtcgcctggtcatgttcaaacggaaaca |
| | ggtgacaccagaacgactcacaggacgggaactgctgcgatcgctgggcattaacggagtag |
| | ttgaaggggaagccaatttctgtgaatacaacggcgaaagatcctgtaaaaaatgccctgattg |
| | cattctctatggttttgcgattggggacagtggctcggaacgttccaaagtctactctgattcagc |
| | cttttccctgagtccttatgaagtttcccatcgcagttttaccttcaatgctccctttgaaggtggaac |
| | catgagtgagaagggtgaaatgcggagtgcgatcaacgaactggatcatgttttgccggaagtt |
| | gcttttccttgcgttgaaacactacgtgaccccacgctggaaggcttcatttatgtaatgggcaac |
| | ctgatccggactcggcgctatggagcgcaggagtcgcgtacagggacaatggcgaatcacat |
| | tattggcgttaccctatgtgatggcgagatcttcagcaatttgcactttacccaagccctctacgat |
| | gccctgaagggtgatttgagtgcgccaattagtgatttatgcagagaggcggcgatcgtggca |
| | gaaaacttgctaaatcaggaacctgtgcggaagatccaagttatttttggagagcagttgaacga |
| | actcctggctgaggtagcgggaatctgccaggatgaaactcgcttgcgaaacactgtgacagc |
| | cttgtaccagcaatcacaagcctatgccgagacctatggtgctgctgctaagaaaggcaaagg |
| | caaatcttaaggtggctgccaatggttatttatcgatgcaatctcacgttgcacgacaacgtattct |
| | tcgccactcgcgagatggggattctctacgaaacagagaagtatctacacaactgggcactca |
| | gctttgctctgtttgaagccacccacatccccaaaccctatcgcttgcagggtgcgatcgctcaa |
| | cgacccggttatctggaagctgacaacgagcaaaaccttttagttctgaatcacgcaggcatcta |
| | tgttttccctgctctaccacttcagtggtcctaccaagtcaataccttcaaagcggctcaagtgcct |
| | tactatggcaaatccaaacagttcggggataagggagctgatcgcaactacccgattaactacg |
| | gacgggcaaaagaactggcagttggcagcacctaccgaacgtatattcttgcccctgaaccaa |
| | tccagatcccccactggatacggttgggtaagtgggcagccaagattcaggtggacgcaaga |
| | cccatacctaactgtggaatccgacaacggtcaggagaatacacctgcgaccatccactaaatc |
| | cgatcgacctgtccggttcaacccgattgctgttatacaaccgcattgtcatgccacccgctagtc |
| | tcgtcagtcagtcgcgagtttctggggactactgggaaatttctaaaccatccaattggactgaat |
| | ttccggactggtcagacctgaaagacatacccaatcaagtttgcttacccagcggagtagcctat |
| | ggagccagtactgccattgccgcatcttaagtccaccgctctgcatatcctggtaattcatttagg |
| | cgttgccgaaagcggagccttgcctgttgaatccggtcgagccgtccatgctcaagttttggact |
| | ggttccgcatggcggatccggcaatttctgaggcaattcacaaaagccaggaatccccgctca |
| | gtctctcaggcctggtcgggaaacgccgtaaagtaggggtgcgggcaggagatgaattttact |
| | tccgaattgggttgttagatgggagtttgattgaaccactgctgagtggtttggagaagtggggc |
| | gacaagcccttttctctggcaaagtgtccctttgttttccgcagtatcaatatgttgccaggaactg |
| | acccctgggttggttcttccgattatgccttgcttacccaaaccccggctactttggataacctagc |
| | tctcaagctcctgtcgccaacaagcttcaagtcgaatgccgggcaaggaatccaaccctttcctt |
| | tgccagagtcagttttcggaaatttacatcgtcgctggaatgtctttgcaccaaaggaattgcagtt |
| | tccaaaaattcaatggagtgggctggtctctgattatgacctcaaaactcaaaagttaaggatgg |
| | aaaactcggttgagatcggcgcagtgggctgggtgcgctatcggtttccagatccagaacaag |
| | cgagaattgcaaccattctggctcactttgccttttttgctggagttgggcgtaagacagcaatgg |
| | ggatgggacaggtggtgttggacaaaaaaacgtagcctaggctgctgccaccgctgagcaat |
| | aactagcataaccccttggggcctctaaacgggtcttgaggggttttttgctgaaacctcaggca |
| | tttgagaagcacacggtcacactgcttccggtagtcaataaaccggtaaaccagcaatagacat |
| | aagcggctatttaacgaccctcgggatctcgacgctctcccttatgcgactcctgcattaggaaa |
| | ttaatacgactcactataggggaattgtgagcggataacaattcccctgtagaaataattttgttta |
| | actttaataaggagatataccatgcttgatgaaaaaagcttcgacaactggtgccaatccgttggt |
| | ctgagccaggcagcacgtgaagttgtagagcgtgtgcgatcctcttcaccctcccgtcgcgtac |
| | agagcggtagaggaaacgtctccggcaattaccctagccgcaaaatgggcgtcacaattcagt |
| | ttgagtcccatcgaaatgagcttgcccgcatctacgaactggagcatgaccccggcgttctgga |
| | atattacgatcaacctcccaaaatcgaacttgattatttgtcaaaaaatgggaggcgtgttcgtact |
| | caatacacacctgatttctttgttatccgtaatgactcagcaggttgggaggagtgtaaaacagag |
| | gatgaccttaagcaattagctgaggacagccctaaccgttaccaacgagctgaggacaatcgc |
| | tggcgatgtccacctgcagaagcctatgctcttccccttgagttgtattttcaggtacggtctagtg |
| | ctgaaattaactggattttccagcaaaactttgtatggctagaagactaccttggtactgaatcactt |
| | actatggatgagtcggttgcagatgcaatcatagccacagtcagagcaaacgagggtatcaca |
| | ctactcgaactgattcaacaattacaaggcacagatgctgatgctatctacatcctgattgcaacc |
| | gaacaaatttatgttgatttgatggctgaacggttagcgaacagcgatcgggcacgggtattttc |
| | cagtccagaaattgctcaagcctttcaactcattacgctggtcgaagcacctctggctaacagttc |
| | tctccatatagttcaggtgggcgttggcacaaccatacactgggatggtgaagtctgggagatt |
| | gttaatacaggctctaccatgacaggattgttgcgatcggacggtaagtttatcgagttgcccaat |
| | tctgcaattgatgaattgattcagaagggcaagattcaatcaatttcagcacaacaagcccctgat |
| | agcaaagcagaagttgaagaaatgcttcgctgtgctcgtccagccgacattaaagaagccaac |
| | cgtcgctacgaaatcattcagccttacctggaaaaggatgcaccagcttgtccaactcgaacaat |
| | tcggcgctggcgtaatcagtattggcaggcagaaaagctttacgggaatggctatgttggtttac |
| | taccaaatcatcagaagaaagggaattatttagttaaggttaatgaagtaactcaagcttttatgctt |
| | gagtttattgagaagcattacgaaaatctcaaacagcgtagtaagcttagagtttatcaggcttttg |
| | tggaggcatgtgaaagccacgaaccgaaactaaaccctccaagccgtactacattcacactag |
| | cgattcagaaacgagcaggacatcagcaaaccttgaaacgtgaaggacgaagagctgccatt |
| | cagaaggaggagttttactgggaactggagctaactactcctcgacacggcgatcgccccttc |
| | gagatcgttcatattgatcacactcaactcgatattgagcttatcagttctttgaactccctggccca |
| | atgcaaccctacatctgttgatgccacttacaatttgggtcgtccttgggcaacgttcatggttgat |
| | gcttacagccgccgtttgctagcagtttacataacctatgaagaacccagctatcgttcctgcatg |
| | atggtgctccgtatttgcgtacagcgatttaagcgttttcctcagacgatcgtggtagataatggtg |
| | cagaatttcacagccattactttgaacaactactggcaacatatcaatgcacaaagaagcacag |
| | gccacctgcaaaagctcgctttggtgcagttgttgaacgattatttggcactgcaaataagcaatt |
| | aattcatgagttgcagggaaatacccaaattactcgcaagcatcgccaagttactcaatctgttca |
| | tccccaaaaattggctctttggacgttaggcgatctctacgaatcactatgtgaatgggcttacaa |
| | agtttatgctgaccgccagcaccctgcgctagggcaaagccctaatgctgcattcgctcaaggg |
| | ctggcgttgggaggtagtcgcatccatcgccgagtagagtatgaccaagtcttccgtatcttgac |
| | attgcccgcaccggaaagaggacaacgcaaagttcagccaggacaagggataaaacttagc |
| | aatatctactactggtcaaatgcctttcgagacccagaaattgaacaatcgatggttgaagttcgtt |
| | atgatccctttgatgccagcattgcctatgcactggtgcgaaaacaatgggtccggtgtatttcct |
| | cctactatcaacagtttcaaaatcgttcagaaaaggaaattcgtttaataactgctgagttgattaa |
| | gcaaaagcgtgaccagggaagagatacaaatgttacggataaagaattggcgcagtttttaag |
| | ctctacagaaatgaaagaaggcaaattcttggagcaacgcttgcggactacagaaaacagggt |
| | agttattcagaatagtacagctacagaagacgatccccccatcactaattctcagaattcttacga |
| | gccatcgcttatgctgccaagtgaggatgagttggatgatcaggaagactggttaaataccggc |
| | aaagctgaggcagaattagaattctatggagagtactagcgatgcaacctcaaataagcttccc |
| | aaaagagttactgcttcaaccaattcctgatcgaattgcctatttcaaagattacacaatggcacat |
| | ccgaagcttatagaagcagaccggaagttaatgcatgctattcgtgaacctggaggagctgctc |
| | ttatctttgtctttggtcctaccggagttggaaagtctactctattgaaacgaattactcaaaagctg |
| | cttaaagaagcattgtctttgatggagactgacaaaggctatattcctattgcgggggtagagata |
| | aaaactccagaggatagtaactttgattggaacgatttttatgttcgtgctcttatggcacttaatga |
| | gccaatgatcgagaaaaaaatcaaccgtacaggtgcaaaactaaaattacggttggcacttgaa |
| | tctgcccttcgtaatcgccgtcctgatgctttttacatagacgaagctcaaaattttggtaaggtag |
| | ccagtgggcgaaaattgcaagatcaaacagattgcattaaatctcttgcaaatctcgctcaaact |
| | cggtttatcttatgtgggatttatgaacttttaccactacgaaatctaagtgggcagctatgccgcc |
| | gtagccatagtattcactttccacgttattcggcagattctgagcaggatctaaaggcctttaaaag |
| | tgcgttattaacactacaaaatcaactccctcttcccgaaccacctgatttagtggcgcattggga |
| | attttgttatgagagaactatcggctgtattggtattttgaaagatttgctccttcggactctaagcg |
| | cgattttagtacgggatggagacaaagttttgacattgaagcccaaggatttagaacaacgtgca |
| | tggtctttagaagaatgtttggttatgctgagagaggcaaaggaaggagagcaacaacttactg |
| | acaaatcgggcttacatgaggaattacggacagctttagggtttaatgcagctacgtccaagca |
| | gtctgccaaatcatccaagtcaacatcaagcaaaaaagcaggcagtgttggaaagccttcacct |
| | cgtcgtcgttcagttggaatagatcaagatgccagctaacgatctagaaatctatgaatcttggaa |
| | tttagatctccctttactacctaatcgaagtaggctctaccacttgaaaccgattgcaattggagat |
| | gcttacgttgaaggattaatcagctatgtttgccgtttggctgaagcccactgtgtttctccaggag |
| | tccttactaaacaggaaatattgcccagtctaagaaaagtttataatgtgggttcaagagaagttc |
| | atgaagtacgaagagatgaaagtttagtttctatttcctcctttcctaagccagaaagcaataaaaa |
| | tcctaatgaaccaggcttctgggcatggcaatatattgaaggattgcagcctttaactcttagaga |
| | aaaccttgaagcattaactattccaaattggaaaaagcaaattccacaggtgacaggaattgag |
| | gttgcgcgtgagttgagagcttggtgccctgaatgctttcaacaatggcgcaagactaatcagat |
| | aatttatgaaccgatgctttggtcgatcgctttagttaccgtatgtccttatcattaccttccgctaca |
| | aactcgctgtccttattgtaaaaaaactcaacggcctatcacaggaaaaatgcaggtagctcgct |
| | gttccgaatgtttagagtggcttggcattcaaccaagcgaagattcagagttagaaatcaaaatta |
| | aatctgaagtcgaatggcaccattgggttgcggagaaagtaatggagattttggtatttaaagatt |
| | atactttaactgcaaatgctataacaatagcagctccaattacgaaagaaaaatattttcaactaga |
| | atcgagcttaataaattgtccaattaagtatgctcaacagcagttcttaaagctcttcttcaattaaat |
| | ttataaaggtatggctatggcgactgaagtaccttttacttttagcacgtgggatttatctaaacccc |
| | ttaccccagctcgtagttctctctttagcttgaaaccgattggggttggaacggctgcagtagaaa |
| | gcctcactggatatattgcacgtttggctgaagctcactgtatctggccaggagatcttcttgcag |
| | ctgaatttagttcttcatattcctattcttacctgcatagcatgaggggccgtattgctgctttgaatg |
| | gtaatagccaaatggctcaggatgctgtacttaagcttcaagagcttaccttacaggagaatttat |
| | ccgctttaacaatgttaggctgggctaatattttacccgcaaaagggttgttaaatcggactcgaa |
| | cttggtgtccggtttgttttgaggcgtggcgtttcatcaaaagtcctatatatgagcctctattatggt |
| | atttaacctcggtcaaatattgtccaattcatcagcaacgccttttatctcaatgtccattctgcgggt |
| | gccagcagcctcctctagactggaaaactcggccgggctattgttcaaggtgccaggtgtggc |
| | tgggttcaccaccaggtgtaacttccagtaattcgctttctaatacagaaataaggtgggagagtt |
| | gggttgcccagagctgcggaaatttgttagcctatagtcctcgtttacacaagatatccgttagcg |
| | ttcaggtcgcagcatccctttctaaatgtatcaacctttatgcagagggcaatattgcggagtttgc |
| | ccgtcaaatgagcttgccaaaaacttctgtttggctgtggcagtctggtcaggtactccccgagtt |
| | atccaggcttttaagcatctgctatcacttcaatctgtcgctcttagactttctaattaatacaccagt |
| | tcttaagataaggagagacttaattccagaaaagagaactaatgagtttcgccagcctcgaagag |
| | atccctcaaccatatctcaattgcaacctattctgggattaattcttttcgagcagccacctccctctt |
| | tagcagcagtagcgtgtcgcttaggttgtagctcaagatccctgcgccataattttcctgatctctg |
| | tggctctatttctgcacgttaccttcaacaccgtgatgaagtttataaagaaaaattgaatcaattct |
| | gtcaagacttgcgccaagttgtacttcaactttattcggcaggcattgatccgactcggagccaa |
| | gttgctcgccgtctgaggaaacccgcttattttagagaacccgaagtcgaggcaatgcttgagt |
| | ctgttcgacgagaattaggcttgcacagcaattctcagcatgagactaattctcacaaaaacgcc |
| | cagttttaacttaagtcgaacagaaagtaatcgtattgtacacggccgcataatcgaaattaatac |
| | gactcactataggggaattgtgagcggataacaattccccatcttagtatattagttaagtataaga |
| | aggagatatacatatggcagatctcaattggatatcggccggccacgcgatcgctgacgtcggt |
| | accctcgagtctggtaaagaaaccgctgctgcgaaatttgaacgccagcacatggactcgtcta |
| | ctagcgcagcttaattaacctaggctgctgccaccgctgagcaataactagcataaccccttgg |
| | ggcctctaaacgggtcttgaggggttttttgctgaaacctcaggcatttgagaagcacacggtca |
| | cactgcttccggtagtgccctgaaccgacgaccgggtcatcgtggccggatcttgcggcccct |
| | cggcttgaacgaattgttagacattatttgccgactaccttggtgatctcgcctttcacgtagtgga |
| | caaattcttccaactgatctgcgcgcgaggccaagcgatcttcttcttgtccaagataagcctgtc |
| | tagcttcaagtatgacgggctgatactgggccggcaggcgctccattgcccagtcggcagcga |
| | catccttcggcgcgattttgccggttactgcgctgtaccaaatggggacaacgtaagcactac |
| | atttcgctcatcgccagcccagtcgggcggcgagttccatagcgttaaggtttcatttagcgcct |
| | caaatagatcctgttcaggaaccggatcaaagagttcctccgccgctggacctaccaaggcaa |
| | cgctatgttctcttgcttttgtcagcaagatagccagatcaatgtcgatcgtggctggctcgaaga |
| | tacctgcaagaatgtcattgcgctgccattctccaaattgcagttcgcgcttagctggataacgcc |
| | acggaatgatgtcgtcgtgcacaacaatggtgacttctacagcgcggagaatctcgctctctcc |
| | aggggaagccgaagtttccaaaaggtcgttgatcaaagctcgccgcgttgtttcatcaagcctta |
| | cggtcaccgtaaccagcaaatcaatatcactgtgtggcttcaggccgccatccactgcggagc |
| | cgtacaaatgtacggccagcaacgtcggttcgagatggcgctcgatgacgccaactacctctg |
| | atagttgagtcgatacttcggcgatcaccgcttccctcatactcttcctttttcaatattattgaagca |
| | tttatcagggttattgtctcatgagcggatacatatttgaatgtatttagaaaaataaacaaatagct |
| | agctcactcggtcgctacgctccgggcgtgagactgcggcgggcgctgcggacacatacaaa |
| | gttacccacagattccgtggataagcaggggactaacatgtgaggcaaaacagcagggccgc |
| | gccggtggcgtttttccataggctccgccctcctgccagagttcacataaacagacgcttttccg |
| | gtgcatctgtgggagccgtgaggctcaaccatgaatctgacagtacgggcgaaacccgacag |
| | gacttaaagatccccaccgtttccggcgggtcgctccctcttgcgctctcctgttccgaccctgc |
| | cgtttaccggatacctgttccgcctttctcccttacgggaagtgtggcgctttctcatagctcacac |
| | actggtatctcggctcggtgtaggtcgttcgctccaagctgggctgtaagcaagaactccccgtt |
| | cagcccgactgctgcgccttatccggtaactgttcacttgagtccaacccggaaaagcacggta |
| | aaacgccactggcagcagccattggtaactgggagttcgcagaggatttgtttagctaaacacg |
| | cggttgctcttgaagtgtgcgccaaagtccggctacactggaaggacagatttggttgctgtgct |
| | ctgcgaaagccagttaccacggttaagcagttccccaactgacttaaccttcgatcaaaccacct |
| | ccccaggtggttttttcgtttacagggcaaaagattacgcgcagaaaaaaaggatctcaagaag |
| | atcctttgatcttttctactgaaccgctctagatttcagtgcaatttatctcttcaaatgtagcacctg |
| | aagtcagccccatacgatataagttgtaattctcatgttagtcatgccccgcgcccaccggaagga |
| | gctgactgggttgaaggctctcaagggcatcggtcgagatcccggtgcctaatgagtgagcta |
| | acttacattaattgcgttgcgctcactgcccgctttccagtcgggaaacctgtcgtgccagctgca |
| | ttaatgaatcggccaacgcgcggggagaggcggtttgcgtattgggcgccagggtggtttttct |
| | tttcaccagtgagacgggcaacagctgattgcccttcaccgcctggccctgagagagttgcag |
| | caagcggtccacgctggtttgccccagcaggcgaaaatcctgtttgatggtggttaacggcgg |
| | gatataacatgagctgtcttcggtatcgtcgtatcccactaccgagatgtccgcaccaacgcgca |
| | gcccggactcggtaatggcgcgcattgcgcccagcgccatctgatcgttggcaaccagcatcg |
| | cagtgggaacgatgccctcattcagcatttgcatggtttgttgaaaaccggacatggcactccag |
| | tcgccttcccgttccgctatcggctgaatttgattgcgagtgagatatttatgccagccagccaga |
| | cgcagacgcgccgagacagaacttaatgggcccgctaacagcgcgatttgctggtgacccaa |
| | tgcgaccagatgctccacgcccagtcgcgtaccgtcttcatgggagaaaataatactgttgatg |
| | ggtgtctggtcagagacatcaagaaataacgccggaacattagtgcaggcagcttccacagca |
| | atggcatcctggtcatccagcggatagttaatgatcagcccactgacgcgttgcgcgagaagat |
| | tgtgcaccgccgctttacaggcttcgacgccgcttcgttctaccatcgacaccaccacgctggc |
| | acccagttgatcggcgcgagatttaatcgccgcgacaatttgcgacggcgcgtgcagggcca |
| | gactggaggtggcaacgccaatcagcaacgactgtttgcccgccagttgttgtgccacgcggt |
| | tgggaatgtaattcagctccgccatcgccgcttccactttttcccgcgttttcgcagaaacgtggc |
| | tggcctggttcaccacgcgggaaacggtctgataagagacaccggcatactctgcgacatcgt |
| | ataacgttactggtttcacattcaccaccctgaattgactctcttccgggcgctatcatgccatacc |
| | gcgaaaggttttgcgccattcgatggtgtccgggatctcgacgctctcccttatgcgactcctgc |
| | attaggaaattaatacgactcactata (SEQ ID NO: 90) |
| pHelper(CyCAST)_ | ggggaattgtgagcggataacaattcccctgtagaaataattttgtttaactttaataaggagatat |
| Shuffled-spacer | accatggtttccaagcctgaatcccggtctacgggactgaaacaccatctatgtatagctacgat |
| | ggaatagtaaagggtttccaagcctgaatcccggtctacgggactgaaacgatccgaattcga |
| | gctcggcgcgcctgcaggtcgacaagcttgcggccgcataatgcttaagtcgaacagaaagta |
| | atcgtattgtacacggccgcataatcgaaattaatacgactcactataggggaattgtgagcgga |
| | taacaattccccatcttagtatattagttaagtataagaaggagatatacatatggctaccctactcc |
| | aaaccctcctgttacgaacgttgtcacccgaggcggatccagttctctatgtctacattgagacg |
| | gtactacctgcaatggaacgggaatttggcttgattccagcccttggtggctcggaggaagcac |
| | actttcatctgctgaggcgacggggaaacaaatatgcggcggaaaattccaagcggtacgcca |
| | gtcgctctgatcaaagcctgctggttcatgtgctcaatgccttgctgaccgcctggaatttgttgc |
| | cattcctaagctccacatttgaactatcagatgatgagaaacggctgctctgcctggggctgacc |
| | ctacacgattacaacaagtggtgccagggtgaggaggaagattcgccgattgcatgcgaagta |
| | gcagagattcttgagctttgtcaaacattgggagagcggttgaactttgacggattctgggcaga |
| | gtggcgggagtatttgacagagattgcctaccttgcccaaaacacccagggaaaagttggaac |
| | aaatctacccaaagcgaattggggcagcttcaaaattctagatacccgtcgtctggattcgcccc |
| | tacgatggctactcgcttttggcgatgtcgcagtccatctcagcgatccatctgacatcgcaatga |
| | aaacggggggagatcgcctgcgggaacatctgcgctttctaggtattcgtcgcaagctggtcta |
| | ccatcgcctgcgcaactgtacagggctattaaccaacggaattcataatgcggtaatgcattttg |
| | ctagagaactggattggcaacccatcctcttctttgctcagggtgttgtttatctcgctcctcaggg |
| | tgctggcgttccggatcgcttagaacttcaggaatttctctgggagcaggtcagcaaccgattag |
| | ccaccagcatgttgcggggagaggttggtttcaaacgggatggcaagggactcaaggggct |
| | ccccaaaccctggaactgttctcccctgctcagttgattcggaacttgcccggtgttgtccaatctt |
| | atgtcggaaacgctaaatctcctgcgactccaaaacgacttgagaagctggaactcagttcaac |
| | agagcgagagtttttggctaaaggtgcagatttaagagcagatcgcattgccgagtttatctttct |
| | ggcgcaaaaagagtttttcggagataaaccagaatttatcacttggatgcttgaaactctggggg |
| | ttcaaactgctatcacttcggagcagacgcagcttcaatccggtggtgtgaactggggatggtat |
| | cacgcagcagcctattacgtcgcgagcaatgccacgctggatttggaagagatttcggagaaa |
| | cttcaggaatttgccgagcagttggcaacctgggcggaggaaaacaatttcttcccagagcatc |
| | gcagcccaacccatgacatctttttcaactatctgactcaatacctggaagttcaggggtgggag |
| | cagcaggagcagtcctttcagcttgaactggcaggctatgttggcgcaaagactaaaactgcca |
| | gacaaccgatttgctccctcagttccggtgaatttgcttcggaagaccagatggattctgttgtcct |
| | gttcaaaccgcagcagtacagcaacaagaatcctctcggtggacggcagatcaagcggggg |
| | atttccaaaatctggtccctggaaatgctgctcaggcaggctctttggtctgcgccagcaggcaa |
| | actagaggaacaacaacccattttcctttacatcttcccggcatacgtttactcgccccaggttgct |
| | gctgctgtccggttactggtgaatgatctgaagcgtgtcaacctctgggaagtccgtaaacactg |
| | gttggagaatggcatggatgctcaggcgttgcgatcgtttccctggctagtcggtgacaaaaac |
| | gattcgggtaactctgctccatccagatactctggtgaagacttgcctttcatggcaatgaactac |
| | accaccaccagaggcaaaacgataactgatgcctgggttgaacctgctttcctgacactggtgc |
| | tgcctttgttgctgggtgtgaaagttgtcgccaccagtagccagattccactctatgaaagcgac |
| | aacgacttttgcgaatctgtcaagctggatggacccgccgatttctgggctttgctgcggctacc |
| | aacttccctgcatctccaggattggattggggggcgtgttcaaaatttagcagaaggcttgaacc |
| | gcttgatggttgcctactgcctgcatctggacagtcgtagcgctccaccagatgcgcgctggag |
| | agccttaaacggcagtgtccgagagatgacaacaaatgttctaaacattttttcacttgcagagg |
| | agggattacgtcgtgatggctctaaacgcgatgcaacagttgaagaaattaaccgctattggaa |
| | atacgcccagctgtgggcagagggagactctgatatgcaaaacaagttgaagattaccaaacg |
| | cttagtcgatgagtaccgccagttttacagagccagtgcgaacgaatccagccatacggttttgc |
| | tgccattttctaaggcactggaacacattctttcggtaccaaatgattgggacgatgaggaaatta |
| | ttgttcaaggggcaggacagttacaggccgcgttagatcggcaggaagtctacaaacgtcccc |
| | tcatgcttgacaaagagattccctttgaaattcgtcggatgaaggagtttgaggcaattcaaacct |
| | tcatgaccacctgcgtcattgacctatttggacaaatgtgtaaaggcgatcgcgcgctcctgcag |
| | gaacatcgcaaccgccttaaatcaggggtcgaatttgcctatcgtcagttagccctgcaagaga |
| | aacaagccaaagccaaccaaaaatctggagaacaatcatgagcattcttgaaaccttaaagcc |
| | agagttccacacagccttccctcgcctcgcctctggcaaatatgtccatttcttcatggtgcgtca |
| | cagccagtcgttccccgtctttcaaaccgatggcgttttgaacactgcccgtactcaggctgggtt |
| | aaatgcaggcgatgaaaaatccagacaggcaatcagtcgcctggtcatgttcaaacggaaaca |
| | ggtgacaccagaacgactcacaggacgggaactgctgcgatcgctgggcattaacggagtag |
| | ttgaaggggaagccaatttctgtgaatacaacggcgaaagatcctgtaaaaaatgccctgattg |
| | cattctctatggttttgcgattggggacagtggctcggaacgttccaaagtctactctgattcagc |
| | cttttccctgagtccttatgaagtttcccatcgcagttttaccttcaatgctccctttgaaggtggaac |
| | catgagtgagaagggtgaaatgcggagtgcgatcaacgaactggatcatgttttgccggaagtt |
| | gcttttccttgcgttgaaacactacgtgaccccacgctggaaggcttcatttatgtaatgggcaac |
| | ctgatccggactcggcgctatggagcgcaggagtcgcgtacagggacaatggcgaatcacat |
| | tattggcgttaccctatgtgatggcgagatcttcagcaatttgcactttacccaagccctctacgat |
| | gccctgaagggtgatttgagtgcgccaattagtgatttatgcagagaggcggcgatcgtggca |
| | gaaaacttgctaaatcaggaacctgtgcggaagatccaagttatttttggagagcagttgaacga |
| | actcctggctgaggtagcgggaatctgccaggatgaaactcgcttgcgaaacactgtgacagc |
| | cttgtaccagcaatcacaagcctatgccgagacctatggtgctgctgctaagaaaggcaaagg |
| | caaatcttaaggtggctgccaatggttatttatcgatgcaatctcacgttgcacgacaacgtattct |
| | tcgccactcgcgagatggggattctctacgaaacagagaagtatctacacaactgggcactca |
| | gctttgctctgtttgaagccacccacatccccaaaccctatcgcttgcagggtgcgatcgctcaa |
| | cgacccggttatctggaagctgacaacgagcaaaaccttttagttctgaatcacgcaggcatcta |
| | tgttttccctgctctaccacttcagtggtcctaccaagtcaataccttcaaagcggctcaagtgcct |
| | tactatggcaaatccaaacagttcggggataagggagctgatcgcaactacccgattaactacg |
| | gacgggcaaaagaactggcagttggcagcacctaccgaacgtatattcttgcccctgaaccaa |
| | tccagatcccccactggatacggttgggtaagtgggcagccaagattcaggtggacgcaaga |
| | cccatacctaactgtggaatccgacaacggtcaggagaatacacctgcgaccatccactaaatc |
| | cgatcgacctgtccggttcaacccgattgctgttatacaaccgcattgtcatgccacccgctagtc |
| | tcgtcagtcagtcgcgagtttctggggactactgggaaatttctaaaccatccaattggactgaat |
| | ttccggactggtcagacctgaaagacatacccaatcaagtttgcttacccagcggagtagcctat |
| | ggagccagtactgccattgccgcatcttaagtccaccgctctgcatatcctggtaattcatttagg |
| | cgttgccgaaagcggagccttgcctgttgaatccggtcgagccgtccatgctcaagttttggact |
| | ggttccgcatggcggatccggcaatttctgaggcaattcacaaaagccaggaatccccgctca |
| | gtctctcaggcctggtcgggaaacgccgtaaagtaggggtgcgggcaggagatgaattttact |
| | tccgaattgggttgttagatgggagtttgattgaaccactgctgagtggtttggagaagtggggc |
| | gacaagcccttttctctggcaaagtgtccctttgttttccgcagtatcaatatgttgccaggaactg |
| | acccctgggttggttcttccgattatgccttgcttacccaaaccccggctactttggataacctagc |
| | tctcaagctcctgtcgccaacaagcttcaagtcgaatgccgggcaaggaatccaaccctttcctt |
| | tgccagagtcagttttcggaaatttacatcgtcgctggaatgtctttgcaccaaaggaattgcagtt |
| | tccaaaaattcaatggagtgggctggtctctgattatgacctcaaaactcaaaagttaaggatgg |
| | aaaactcggttgagatcggcgcagtgggctgggtgcgctatcggtttccagatccagaacaag |
| | cgagaattgcaaccattctggctcactttgccttttttgctggagttgggcgtaagacagcaatgg |
| | ggatgggacaggtggtgttggacaaaaaaacgtagcctaggctgctgccaccgctgagcaat |
| | aactagcataaccccttggggcctctaaacgggtcttgaggggttttttgctgaaacctcaggca |
| | tttgagaagcacacggtcacactgcttccggtagtcaataaaccggtaaaccagcaatagacat |
| | aagcggctatttaacgaccctcgggatctcgacgctctcccttatgcgactcctgcattaggaaa |
| | ttaatacgactcactataggggaattgtgagcggataacaattcccctgtagaaataattttgttta |
| | actttaataaggagatataccatgcttgatgaaaaaagcttcgacaactggtgccaatccgttggt |
| | ctgagccaggcagcacgtgaagttgtagagcgtgtgcgatcctcttcaccctcccgtcgcgtac |
| | agagcggtagaggaaacgtctccggcaattaccctagccgcaaaatgggcgtcacaattcagt |
| | ttgagtcccatcgaaatgagcttgcccgcatctacgaactggagcatgaccccggcgttctgga |
| | atattacgatcaacctcccaaaatcgaacttgattatttgtcaaaaaatgggaggcgtgttcgtact |
| | caatacacacctgatttctttgttatccgtaatgactcagcaggttgggaggagtgtaaaacagag |
| | gatgaccttaagcaattagctgaggacagccctaaccgttaccaacgagctgaggacaatcgc |
| | tggcgatgtccacctgcagaagcctatgctcttccccttgagttgtattttcaggtacggtctagtg |
| | ctgaaattaactggattttccagcaaaactttgtatggctagaagactaccttggtactgaatcactt |
| | actatggatgagtcggttgcagatgcaatcatagccacagtcagagcaaacgagggtatcaca |
| | ctactcgaactgattcaacaattacaaggcacagatgctgatgctatctacatcctgattgcaacc |
| | gaacaaatttatgttgatttgatggctgaacggttagcgaacagcgatcgggcacgggtattttc |
| | cagtccagaaattgctcaagcctttcaactcattacgctggtcgaagcacctctggctaacagttc |
| | tctccatatagttcaggtgggcgttggcacaaccatacactgggatggtgaagtctgggagatt |
| | gttaatacaggctctaccatgacaggattgttgcgatcggacggtaagtttatcgagttgcccaat |
| | tctgcaattgatgaattgattcagaagggcaagattcaatcaatttcagcacaacaagcccctgat |
| | agcaaagcagaagttgaagaaatgcttcgctgtgctcgtccagccgacattaaagaagccaac |
| | cgtcgctacgaaatcattcagccttacctggaaaaggatgcaccagcttgtccaactcgaacaat |
| | tcggcgctggcgtaatcagtattggcaggcagaaaagctttacgggaatggctatgttggtttac |
| | taccaaatcatcagaagaaagggaattatttagttaaggttaatgaagtaactcaagcttttatgctt |
| | gagtttattgagaagcattacgaaaatctcaaacagcgtagtaagcttagagtttatcaggcttttg |
| | tggaggcatgtgaaagccacgaaccgaaactaaaccctccaagccgtactacattcacactag |
| | cgattcagaaacgagcaggacatcagcaaaccttgaaacgtgaaggacgaagagctgccatt |
| | cagaaggaggagttttactgggaactggagctaactactcctcgacacggcgatcgccccttc |
| | gagatcgttcatattgatcacactcaactcgatattgagcttatcagttctttgaactccctggccca |
| | atgcaaccctacatctgttgatgccacttacaatttgggtcgtccttgggcaacgttcatggttgat |
| | gcttacagccgccgtttgctagcagtttacataacctatgaagaacccagctatcgttcctgcatg |
| | atggtgctccgtatttgcgtacagcgatttaagcgttttcctcagacgatcgtggtagataatggtg |
| | cagaatttcacagccattactttgaacaactactggcaacatatcaatgcacaaagaagcacag |
| | gccacctgcaaaagctcgctttggtgcagttgttgaacgattatttggcactgcaaataagcaatt |
| | aattcatgagttgcagggaaatacccaaattactcgcaagcatcgccaagttactcaatctgttca |
| | tccccaaaaattggctctttggacgttaggcgatctctacgaatcactatgtgaatgggcttacaa |
| | agtttatgctgaccgccagcaccctgcgctagggcaaagccctaatgctgcattcgctcaaggg |
| | ctggcgttgggaggtagtcgcatccatcgccgagtagagtatgaccaagtcttccgtatcttgac |
| | attgcccgcaccggaaagaggacaacgcaaagttcagccaggacaagggataaaacttagc |
| | aatatctactactggtcaaatgcctttcgagacccagaaattgaacaatcgatggttgaagttcgtt |
| | atgatccctttgatgccagcattgcctatgcactggtgcgaaaacaatgggtccggtgtatttcct |
| | cctactatcaacagtttcaaaatcgttcagaaaaggaaattcgtttaataactgctgagttgattaa |
| | gcaaaagcgtgaccagggaagagatacaaatgttacggataaagaattggcgcagtttttaag |
| | ctctacagaaatgaaagaaggcaaattcttggagcaacgcttgcggactacagaaaacagggt |
| | agttattcagaatagtacagctacagaagacgatccccccatcactaattctcagaattcttacga |
| | gccatcgcttatgctgccaagtgaggatgagttggatgatcaggaagactggttaaataccggc |
| | aaagctgaggcagaattagaattctatggagagtactagcgatgcaacctcaaataagcttccc |
| | aaaagagttactgcttcaaccaattcctgatcgaattgcctatttcaaagattacacaatggcacat |
| | ccgaagcttatagaagcagaccggaagttaatgcatgctattcgtgaacctggaggagctgctc |
| | ttatctttgtctttggtcctaccggagttggaaagtctactctattgaaacgaattactcaaaagctg |
| | cttaaagaagcattgtctttgatggagactgacaaaggctatattcctattgcgggggtagagata |
| | aaaactccagaggatagtaactttgattggaacgatttttatgttcgtgctcttatggcacttaatga |
| | gccaatgatcgagaaaaaaatcaaccgtacaggtgcaaaactaaaattacggttggcacttgaa |
| | tctgcccttcgtaatcgccgtcctgatgctttttacatagacgaagctcaaaattttggtaaggtag |
| | ccagtgggcgaaaattgcaagatcaaacagattgcattaaatctcttgcaaatctcgctcaaact |
| | cggtttatcttatgtgggatttatgaacttttaccactacgaaatctaagtgggcagctatgccgcc |
| | gtagccatagtattcactttccacgttattcggcagattctgagcaggatctaaaggcctttaaaag |
| | tgcgttattaacactacaaaatcaactccctcttcccgaaccacctgatttagtggcgcattggga |
| | attttgttatgagagaactatcggctgtattggtattttgaaagatttgctccttcggactctaagcg |
| | cgattttagtacgggatggagacaaagttttgacattgaagcccaaggatttagaacaacgtgca |
| | tggtctttagaagaatgtttggttatgctgagagaggcaaaggaaggagagcaacaacttactg |
| | acaaatcgggcttacatgaggaattacggacagctttagggtttaatgcagctacgtccaagca |
| | gtctgccaaatcatccaagtcaacatcaagcaaaaaagcaggcagtgttggaaagccttcacct |
| | cgtcgtcgttcagttggaatagatcaagatgccagctaacgatctagaaatctatgaatcttggaa |
| | tttagatctccctttactacctaatcgaagtaggctctaccacttgaaaccgattgcaattggagat |
| | gcttacgttgaaggattaatcagctatgtttgccgtttggctgaagcccactgtgtttctccaggag |
| | tccttactaaacaggaaatattgcccagtctaagaaaagtttataatgtgggttcaagagaagttc |
| | atgaagtacgaagagatgaaagtttagtttctatttcctcctttcctaagccagaaagcaataaaaa |
| | tcctaatgaaccaggcttctgggcatggcaatatattgaaggattgcagcctttaactcttagaga |
| | aaaccttgaagcattaactattccaaattggaaaaagcaaattccacaggtgacaggaattgag |
| | gttgcgcgtgagttgagagcttggtgccctgaatgctttcaacaatggcgcaagactaatcagat |
| | aatttatgaaccgatgctttggtcgatcgctttagttaccgtatgtccttatcattaccttccgctaca |
| | aactcgctgtccttattgtaaaaaaactcaacggcctatcacaggaaaaatgcaggtagctcgct |
| | gttccgaatgtttagagtggcttggcattcaaccaagcgaagattcagagttagaaatcaaaatta |
| | aatctgaagtcgaatggcaccattgggttgcggagaaagtaatggagattttggtatttaaagatt |
| | atactttaactgcaaatgctataacaatagcagctccaattacgaaagaaaaatattttcaactaga |
| | atcgagcttaataaattgtccaattaagtatgctcaacagcagttcttaaagctcttcttcaattaaat |
| | ttataaaggtatggctatggcgactgaagtaccttttacttttagcacgtgggatttatctaaacccc |
| | ttaccccagctcgtagttctctctttagcttgaaaccgattggggttggaacggctgcagtagaaa |
| | gcctcactggatatattgcacgtttggctgaagctcactgtatctggccaggagatcttcttgcag |
| | ctgaatttagttcttcatattcctattcttacctgcatagcatgaggggccgtattgctgctttgaatg |
| | gtaatagccaaatggctcaggatgctgtacttaagcttcaagagcttaccttacaggagaatttat |
| | ccgctttaacaatgttaggctgggctaatattttacccgcaaaagggttgttaaatcggactcgaa |
| | cttggtgtccggtttgttttgaggcgtggcgtttcatcaaaagtcctatatatgagcctctattatggt |
| | atttaacctcggtcaaatattgtccaattcatcagcaacgccttttatctcaatgtccattctgcgggt |
| | gccagcagcctcctctagactggaaaactcggccgggctattgttcaaggtgccaggtgtggc |
| | tgggttcaccaccaggtgtaacttccagtaattcgctttctaatacagaaataaggtgggagagtt |
| | gggttgcccagagctgcggaaatttgttagcctatagtcctcgtttacacaagatatccgttagcg |
| | ttcaggtcgcagcatccctttctaaatgtatcaacctttatgcagagggcaatattgcggagtttgc |
| | ccgtcaaatgagcttgccaaaaacttctgtttggctgtggcagtctggtcaggtactccccgagtt |
| | atccaggcttttaagcatctgctatcacttcaatctgtcgctcttagactttctaattaatacaccagt |
| | tcttaagataaggagagacttaattccagaaaagagaactaatgagtttcgccagcctcgaagag |
| | atccctcaaccatatctcaattgcaacctattctgggattaattcttttcgagcagccacctccctctt |
| | tagcagcagtagcgtgtcgcttaggttgtagctcaagatccctgcgccataattttcctgatctctg |
| | tggctctatttctgcacgttaccttcaacaccgtgatgaagtttataaagaaaaattgaatcaattct |
| | gtcaagacttgcgccaagttgtacttcaactttattcggcaggcattgatccgactcggagccaa |
| | gttgctcgccgtctgaggaaacccgcttattttagagaacccgaagtcgaggcaatgcttgagt |
| | ctgttcgacgagaattaggcttgcacagcaattctcagcatgagactaattctcacaaaaacgcc |
| | cagttttaacttaagtcgaacagaaagtaatcgtattgtacacggccgcataatcgaaattaatac |
| | gactcactataggggaattgtgagcggataacaattccccatcttagtatattagttaagtataaga |
| | aggagatatacatatggcagatctcaattggatatcggccggccacgcgatcgctgacgtcggt |
| | accctcgagtctggtaaagaaaccgctgctgcgaaatttgaacgccagcacatggactcgtcta |
| | ctagcgcagcttaattaacctaggctgctgccaccgctgagcaataactagcataaccccttgg |
| | ggcctctaaacgggtcttgaggggttttttgctgaaacctcaggcatttgagaagcacacggtca |
| | cactgcttccggtagtgccctgaaccgacgaccgggtcatcgtggccggatcttgcggcccct |
| | cggcttgaacgaattgttagacattatttgccgactaccttggtgatctcgcctttcacgtagtgga |
| | caaattcttccaactgatctgcgcgcgaggccaagcgatcttcttcttgtccaagataagcctgtc |
| | tagcttcaagtatgacgggctgatactgggccggcaggcgctccattgcccagtcggcagcga |
| | catccttcggcgcgattttgccggttactgcgctgtaccaaatgcgggacaacgtaagcactac |
| | atttcgctcatcgccagcccagtcgggcggcgagttccatagcgttaaggtttcatttagcgcct |
| | caaatagatcctgttcaggaaccggatcaaagagttcctccgccgctggacctaccaaggcaa |
| | cgctatgttctcttgcttttgtcagcaagatagccagatcaatgtcgatcgtggctggctcgaaga |
| | tacctgcaagaatgtcattgcgctgccattctccaaattgcagttcgcgcttagctggataacgcc |
| | acggaatgatgtcgtcgtgcacaacaatggtgacttctacagcgcggagaatctcgctctctcc |
| | aggggaagccgaagtttccaaaaggtcgttgatcaaagctcgccgcgttgtttcatcaagcctta |
| | cggtcaccgtaaccagcaaatcaatatcactgtgtggcttcaggccgccatccactgcggagc |
| | cgtacaaatgtacggccagcaacgtcggttcgagatggcgctcgatgacgccaactacctctg |
| | atagttgagtcgatacttcggcgatcaccgcttccctcatactcttcctttttcaatattattgaagca |
| | tttatcagggttattgtctcatgagcggatacatatttgaatgtatttagaaaaataaacaaatagct |
| | agctcactcggtcgctacgctccgggcgtgagactgcgggggcgctgcggacacatacaaa |
| | gttacccacagattccgtggataagcaggggactaacatgtgaggcaaaacagcagggccgc |
| | gccggtggcgtttttccataggctccgccctcctgccagagttcacataaacagacgcttttccg |
| | gtgcatctgtgggagccgtgaggctcaaccatgaatctgacagtacgggcgaaacccgacag |
| | gacttaaagatccccaccgtttccggcgggtcgctccctcttgcgctctcctgttccgaccctgc |
| | cgtttaccggatacctgttccgcctttctcccttacgggaagtgtggcgctttctcatagctcacac |
| | actggtatctcggctcggtgtaggtcgttcgctccaagctgggctgtaagcaagaactccccgtt |
| | cagcccgactgctgcgccttatccggtaactgttcacttgagtccaacccggaaaagcacggta |
| | aaacgccactggcagcagccattggtaactgggagttcgcagaggatttgtttagctaaacacg |
| | cggttgctcttgaagtgtgcgccaaagtccggctacactggaaggacagatttggttgctgtgct |
| | ctgcgaaagccagttaccacggttaagcagttccccaactgacttaaccttcgatcaaaccacct |
| | ccccaggtggttttttcgtttacagggcaaaagattacgcgcagaaaaaaaggatctcaagaag |
| | atcctttgatcttttctactgaaccgctctagatttcagtgcaatttatctcttcaaatgtagcacctg |
| | aagtcagccccatacgatataagttgtaattctcatgttagtcatgccccgcgcccaccggaagga |
| | gctgactgggttgaaggctctcaagggcatcggtcgagatcccggtgcctaatgagtgagcta |
| | acttacattaattgcgttgcgctcactgcccgctttccagtcgggaaacctgtcgtgccagctgca |
| | ttaatgaatcggccaacgcgcggggagaggcggtttgcgtattgggcgccagggtggtttttct |
| | tttcaccagtgagacgggcaacagctgattgcccttcaccgcctggccctgagagagttgcag |
| | caagcggtccacgctggtttgccccagcaggcgaaaatcctgtttgatggtggttaacggcgg |
| | gatataacatgagctgtcttcggtatcgtcgtatcccactaccgagatgtccgcaccaacgcgca |
| | gcccggactcggtaatggcgcgcattgcgcccagcgccatctgatcgttggcaaccagcatcg |
| | cagtgggaacgatgccctcattcagcatttgcatggtttgttgaaaaccggacatggcactccag |
| | tcgccttcccgttccgctatcggctgaatttgattgcgagtgagatatttatgccagccagccaga |
| | cgcagacgcgccgagacagaacttaatgggcccgctaacagcgcgatttgctggtgacccaa |
| | tgcgaccagatgctccacgcccagtcgcgtaccgtcttcatgggagaaaataatactgttgatg |
| | ggtgtctggtcagagacatcaagaaataacgccggaacattagtgcaggcagcttccacagca |
| | atggcatcctggtcatccagcggatagttaatgatcagcccactgacgcgttgcgcgagaagat |
| | tgtgcaccgccgctttacaggcttcgacgccgcttcgttctaccatcgacaccaccacgctggc |
| | acccagttgatcggcgcgagatttaatcgccgcgacaatttgcgacggcgcgtgcagggcca |
| | gactggaggtggcaacgccaatcagcaacgactgtttgcccgccagttgttgtgccacgcggt |
| | tgggaatgtaattcagctccgccatcgccgcttccactttttcccgcgttttcgcagaaacgtggc |
| | tggcctggttcaccacgcgggaaacggtctgataagagacaccggcatactctgcgacatcgt |
| | ataacgttactggtttcacattcaccaccctgaattgactctcttccgggcgctatcatgccatacc |
| | gcgaaaggttttgcgccattcgatggtgtccgggatctcgacgctctcccttatgcgactcctgc |
| | attaggaaattaatacgactcactata (SEQ ID NO: 91) |
| pHelper(CyCAST)_PSP | ggggaattgtgagcggataacaattcccctgtagaaataattttgtttaactttaataaggagatat |
| 1_ΔTniQ | accatggtttccaagcctgaatcccggtctacgggactgaaacgagaagtcatttaataaggcc |
| | actgttaaacgtaggtttccaagcctgaatcccggtctacgggactgaaacgatccgaattcga |
| | gctcggcgcgcctgcaggtcgacaagcttgcggccgcataatgcttaagtcgaacagaaagta |
| | atcgtattgtacacggccgcataatcgaaattaatacgactcactataggggaattgtgagcgga |
| | taacaattccccatcttagtatattagttaagtataagaaggagatatacatatggctaccctactcc |
| | aaaccctcctgttacgaacgttgtcacccgaggcggatccagttctctatgtctacattgagacg |
| | gtactacctgcaatggaacgggaatttggcttgattccagcccttggtggctcggaggaagcac |
| | actttcatctgctgaggcgacggggaaacaaatatgcggcggaaaattccaagcggtacgcca |
| | gtcgctctgatcaaagcctgctggttcatgtgctcaatgccttgctgaccgcctggaatttgttgc |
| | cattcctaagctccacatttgaactatcagatgatgagaaacggctgctctgcctggggctgacc |
| | ctacacgattacaacaagtggtgccagggtgaggaggaagattcgccgattgcatgcgaagta |
| | gcagagattcttgagctttgtcaaacattgggagagcggttgaactttgacggattctgggcaga |
| | gtggcgggagtatttgacagagattgcctaccttgcccaaaacacccagggaaaagttggaac |
| | aaatctacccaaagcgaattggggcagcttcaaaattctagatacccgtcgtctggattcgcccc |
| | tacgatggctactcgcttttggcgatgtcgcagtccatctcagcgatccatctgacatcgcaatga |
| | aaacggggggagatcgcctgcgggaacatctgcgctttctaggtattcgtcgcaagctggtcta |
| | ccatcgcctgcgcaactgtacagggctattaaccaacggaattcataatgcggtaatgcattttg |
| | ctagagaactggattggcaacccatcctcttctttgctcagggtgttgtttatctcgctcctcaggg |
| | tgctggcgttccggatcgcttagaacttcaggaatttctctgggagcaggtcagcaaccgattag |
| | ccaccagcatgttgcggggagaggttggtttcaaacgggatggcaagggactcaaggtggct |
| | ccccaaaccctggaactgttctcccctgctcagttgattcggaacttgcccggtgttgtccaatctt |
| | atgtcggaaacgctaaatctcctgcgactccaaaacgacttgagaagctggaactcagttcaac |
| | agagcgagagtttttggctaaaggtgcagatttaagagcagatcgcattgccgagtttatctttct |
| | ggcgcaaaaagagtttttcggagataaaccagaatttatcacttggatgcttgaaactctggggg |
| | ttcaaactgctatcacttcggagcagacgcagcttcaatccggtggtgtgaactggggatggtat |
| | cacgcagcagcctattacgtcgcgagcaatgccacgctggatttggaagagatttcggagaaa |
| | cttcaggaatttgccgagcagttggcaacctgggcggaggaaaacaatttcttcccagagcatc |
| | gcagcccaacccatgacatctttttcaactatctgactcaatacctggaagttcaggggtgggag |
| | cagcaggagcagtcctttcagcttgaactggcaggctatgttggcgcaaagactaaaactgcca |
| | gacaaccgatttgctccctcagttccggtgaatttgcttcggaagaccagatggattctgttgtcct |
| | gttcaaaccgcagcagtacagcaacaagaatcctctcggtggacggcagatcaagcggggg |
| | atttccaaaatctggtccctggaaatgctgctcaggcaggctctttggtctgcgccagcaggcaa |
| | actagaggaacaacaacccattttcctttacatcttcccggcatacgtttactcgccccaggttgct |
| | gctgctgtccggttactggtgaatgatctgaagcgtgtcaacctctgggaagtccgtaaacactg |
| | gttggagaatggcatggatgctcaggcgttgcgatcgtttccctggctagtcggtgacaaaaac |
| | gattcgggtaactctgctccatccagatactctggtgaagacttgcctttcatggcaatgaactac |
| | accaccaccagaggcaaaacgataactgatgcctgggttgaacctgctttcctgacactggtgc |
| | tgcctttgttgctgggtgtgaaagttgtcgccaccagtagccagattccactctatgaaagcgac |
| | aacgacttttgcgaatctgtcaagctggatggacccgccgatttctgggctttgctgcggctacc |
| | aacttccctgcatctccaggattggattggggggcgtgttcaaaatttagcagaaggcttgaacc |
| | gcttgatggttgcctactgcctgcatctggacagtcgtagcgctccaccagatgcgcgctggag |
| | agccttaaacggcagtgtccgagagatgacaacaaatgttctaaacattttttcacttgcagagg |
| | agggattacgtcgtgatggctctaaacgcgatgcaacagttgaagaaattaaccgctattggaa |
| | atacgcccagctgtgggcagagggagactctgatatgcaaaacaagttgaagattaccaaacg |
| | cttagtcgatgagtaccgccagttttacagagccagtgcgaacgaatccagccatacggttttgc |
| | tgccattttctaaggcactggaacacattctttcggtaccaaatgattgggacgatgaggaaatta |
| | ttgttcaaggggcaggacagttacaggccgcgttagatcggcaggaagtctacaaacgtcccc |
| | tcatgcttgacaaagagattccctttgaaattcgtcggatgaaggagtttgaggcaattcaaacct |
| | tcatgaccacctgcgtcattgacctatttggacaaatgtgtaaaggcgatcgcgcgctcctgcag |
| | gaacatcgcaaccgccttaaatcaggggtcgaatttgcctatcgtcagttagccctgcaagaga |
| | aacaagccaaagccaaccaaaaatctggagaacaatcatgagcattcttgaaaccttaaagcc |
| | agagttccacacagccttccctcgcctcgcctctggcaaatatgtccatttcttcatggtgcgtca |
| | cagccagtcgttccccgtctttcaaaccgatggcgttttgaacactgcccgtactcaggctgggtt |
| | aaatgcaggcgatgaaaaatccagacaggcaatcagtcgcctggtcatgttcaaacggaaaca |
| | ggtgacaccagaacgactcacaggacgggaactgctgcgatcgctgggcattaacggagtag |
| | ttgaaggggaagccaatttctgtgaatacaacggcgaaagatcctgtaaaaaatgccctgattg |
| | cattctctatggttttgcgattggggacagtggctcggaacgttccaaagtctactctgattcagc |
| | cttttccctgagtccttatgaagtttcccatcgcagttttaccttcaatgctccctttgaaggtggaac |
| | catgagtgagaagggtgaaatgcggagtgcgatcaacgaactggatcatgttttgccggaagtt |
| | gcttttccttgcgttgaaacactacgtgaccccacgctggaaggcttcatttatgtaatgggcaac |
| | ctgatccggactcggcgctatggagcgcaggagtcgcgtacagggacaatggcgaatcacat |
| | tattggcgttaccctatgtgatggcgagatcttcagcaatttgcactttacccaagccctctacgat |
| | gccctgaagggtgatttgagtgcgccaattagtgatttatgcagagaggcggcgatcgtggca |
| | gaaaacttgctaaatcaggaacctgtgcggaagatccaagttatttttggagagcagttgaacga |
| | actcctggctgaggtagcgggaatctgccaggatgaaactcgcttgcgaaacactgtgacagc |
| | cttgtaccagcaatcacaagcctatgccgagacctatggtgctgctgctaagaaaggcaaagg |
| | caaatcttaaggtggctgccaatggttatttatcgatgcaatctcacgttgcacgacaacgtattct |
| | tcgccactcgcgagatggggattctctacgaaacagagaagtatctacacaactgggcactca |
| | gctttgctctgtttgaagccacccacatccccaaaccctatcgcttgcagggtgcgatcgctcaa |
| | cgacccggttatctggaagctgacaacgagcaaaaccttttagttctgaatcacgcaggcatcta |
| | tgttttccctgctctaccacttcagtggtcctaccaagtcaataccttcaaagcggctcaagtgcct |
| | tactatggcaaatccaaacagttcggggataagggagctgatcgcaactacccgattaactacg |
| | gacgggcaaaagaactggcagttggcagcacctaccgaacgtatattcttgcccctgaaccaa |
| | tccagatcccccactggatacggttgggtaagtgggcagccaagattcaggtggacgcaaga |
| | cccatacctaactgtggaatccgacaacggtcaggagaatacacctgcgaccatccactaaatc |
| | cgatcgacctgtccggttcaacccgattgctgttatacaaccgcattgtcatgccacccgctagtc |
| | tcgtcagtcagtcgcgagtttctggggactactgggaaatttctaaaccatccaattggactgaat |
| | ttccggactggtcagacctgaaagacatacccaatcaagtttgcttacccagcggagtagcctat |
| | ggagccagtactgccattgccgcatcttaagtccaccgctctgcatatcctggtaattcatttagg |
| | cgttgccgaaagcggagccttgcctgttgaatccggtcgagccgtccatgctcaagttttggact |
| | ggttccgcatggcggatccggcaatttctgaggcaattcacaaaagccaggaatccccgctca |
| | gtctctcaggcctggtcgggaaacgccgtaaagtaggggtgcgggcaggagatgaattttact |
| | tccgaattgggttgttagatgggagtttgattgaaccactgctgagtggtttggagaagtggggc |
| | gacaagcccttttctctggcaaagtgtccctttgttttccgcagtatcaatatgttgccaggaactg |
| | acccctgggttggttcttccgattatgccttgcttacccaaaccccggctactttggataacctagc |
| | tctcaagctcctgtcgccaacaagcttcaagtcgaatgccgggcaaggaatccaaccctttcctt |
| | tgccagagtcagttttcggaaatttacatcgtcgctggaatgtctttgcaccaaaggaattgcagtt |
| | tccaaaaattcaatggagtgggctggtctctgattatgacctcaaaactcaaaagttaaggatgg |
| | aaaactcggttgagatcggcgcagtgggctgggtgcgctatcggtttccagatccagaacaag |
| | cgagaattgcaaccattctggctcactttgccttttttgctggagttgggcgtaagacagcaatgg |
| | ggatgggacaggtggtgttggacaaaaaaacgtagcctaggctgctgccaccgctgagcaat |
| | aactagcataaccccttggggcctctaaacgggtcttgaggggttttttgctgaaacctcaggca |
| | tttgagaagcacacggtcacactgcttccggtagtcaataaaccggtaaaccagcaatagacat |
| | aagcggctatttaacgaccctcgggatctcgacgctctcccttatgcgactcctgcattaggaaa |
| | ttaatacgactcactataggggaattgtgagcggataacaattcccctgtagaaataattttgttta |
| | actttaataaggagatataccatgcttgatgaaaaaagcttcgacaactggtgccaatccgttggt |
| | ctgagccaggcagcacgtgaagttgtagagcgtgtgcgatcctcttcaccctcccgtcgcgtac |
| | agagcggtagaggaaacgtctccggcaattaccctagccgcaaaatgggcgtcacaattcagt |
| | ttgagtcccatcgaaatgagcttgcccgcatctacgaactggagcatgaccccggcgttctgga |
| | atattacgatcaacctcccaaaatcgaacttgattatttgtcaaaaaatgggaggcgtgttcgtact |
| | caatacacacctgatttctttgttatccgtaatgactcagcaggttgggaggagtgtaaaacagag |
| | gatgaccttaagcaattagctgaggacagccctaaccgttaccaacgagctgaggacaatcgc |
| | tggcgatgtccacctgcagaagcctatgctcttccccttgagttgtattttcaggtacggtctagtg |
| | ctgaaattaactggattttccagcaaaactttgtatggctagaagactaccttggtactgaatcactt |
| | actatggatgagtcggttgcagatgcaatcatagccacagtcagagcaaacgagggtatcaca |
| | ctactcgaactgattcaacaattacaaggcacagatgctgatgctatctacatcctgattgcaacc |
| | gaacaaatttatgttgatttgatggctgaacggttagcgaacagcgatcgggcacgggtattttc |
| | cagtccagaaattgctcaagcctttcaactcattacgctggtcgaagcacctctggctaacagttc |
| | tctccatatagttcaggtgggcgttggcacaaccatacactgggatggtgaagtctgggagatt |
| | gttaatacaggctctaccatgacaggattgttgcgatcggacggtaagtttatcgagttgcccaat |
| | tctgcaattgatgaattgattcagaagggcaagattcaatcaatttcagcacaacaagcccctgat |
| | agcaaagcagaagttgaagaaatgcttcgctgtgctcgtccagccgacattaaagaagccaac |
| | cgtcgctacgaaatcattcagccttacctggaaaaggatgcaccagcttgtccaactcgaacaat |
| | tcggcgctggcgtaatcagtattggcaggcagaaaagctttacgggaatggctatgttggtttac |
| | taccaaatcatcagaagaaagggaattatttagttaaggttaatgaagtaactcaagcttttatgctt |
| | gagtttattgagaagcattacgaaaatctcaaacagcgtagtaagcttagagtttatcaggcttttg |
| | tggaggcatgtgaaagccacgaaccgaaactaaaccctccaagccgtactacattcacactag |
| | cgattcagaaacgagcaggacatcagcaaaccttgaaacgtgaaggacgaagagctgccatt |
| | cagaaggaggagttttactgggaactggagctaactactcctcgacacggcgatcgccccttc |
| | gagatcgttcatattgatcacactcaactcgatattgagcttatcagttctttgaactccctggccca |
| | atgcaaccctacatctgttgatgccacttacaatttgggtcgtccttgggcaacgttcatggttgat |
| | gcttacagccgccgtttgctagcagtttacataacctatgaagaacccagctatcgttcctgcatg |
| | atggtgctccgtatttgcgtacagcgatttaagcgttttcctcagacgatcgtggtagataatggtg |
| | cagaatttcacagccattactttgaacaactactggcaacatatcaatgcacaaagaagcacag |
| | gccacctgcaaaagctcgctttggtgcagttgttgaacgattatttggcactgcaaataagcaatt |
| | aattcatgagttgcagggaaatacccaaattactcgcaagcatcgccaagttactcaatctgttca |
| | tccccaaaaattggctctttggacgttaggcgatctctacgaatcactatgtgaatgggcttacaa |
| | agtttatgctgaccgccagcaccctgcgctagggcaaagccctaatgctgcattcgctcaaggg |
| | ctggcgttgggaggtagtcgcatccatcgccgagtagagtatgaccaagtcttccgtatcttgac |
| | attgcccgcaccggaaagaggacaacgcaaagttcagccaggacaagggataaaacttagc |
| | aatatctactactggtcaaatgcctttcgagacccagaaattgaacaatcgatggttgaagttcgtt |
| | atgatccctttgatgccagcattgcctatgcactggtgcgaaaacaatgggtccggtgtatttcct |
| | cctactatcaacagtttcaaaatcgttcagaaaaggaaattcgtttaataactgctgagttgattaa |
| | gcaaaagcgtgaccagggaagagatacaaatgttacggataaagaattggcgcagtttttaag |
| | ctctacagaaatgaaagaaggcaaattcttggagcaacgcttgcggactacagaaaacagggt |
| | agttattcagaatagtacagctacagaagacgatccccccatcactaattctcagaattcttacga |
| | gccatcgcttatgctgccaagtgaggatgagttggatgatcaggaagactggttaaataccggc |
| | aaagctgaggcagaattagaattctatggagagtactagcgatgcaacctcaaataagcttccc |
| | aaaagagttactgcttcaaccaattcctgatcgaattgcctatttcaaagattacacaatggcacat |
| | ccgaagcttatagaagcagaccggaagttaatgcatgctattcgtgaacctggaggagctgctc |
| | ttatctttgtctttggtcctaccggagttggaaagtctactctattgaaacgaattactcaaaagctg |
| | cttaaagaagcattgtctttgatggagactgacaaaggctatattcctattgcgggggtagagata |
| | aaaactccagaggatagtaactttgattggaacgatttttatgttcgtgctcttatggcacttaatga |
| | gccaatgatcgagaaaaaaatcaaccgtacaggtgcaaaactaaaattacggttggcacttgaa |
| | tctgcccttcgtaatcgccgtcctgatgctttttacatagacgaagctcaaaattttggtaaggtag |
| | ccagtgggcgaaaattgcaagatcaaacagattgcattaaatctcttgcaaatctcgctcaaact |
| | cggtttatcttatgtgggatttatgaacttttaccactacgaaatctaagtgggcagctatgccgcc |
| | gtagccatagtattcactttccacgttattcggcagattctgagcaggatctaaaggcctttaaaag |
| | tgcgttattaacactacaaaatcaactccctcttcccgaaccacctgatttagtggcgcattggga |
| | attttgttatgagagaactatcggctgtattggtattttgaaagatttgctccttcggactctaagcg |
| | cgattttagtacgggatggagacaaagttttgacattgaagcccaaggatttagaacaacgtgca |
| | tggtctttagaagaatgtttggttatgctgagagaggcaaaggaaggagagcaacaacttactg |
| | acaaatcgggcttacatgaggaattacggacagctttagggtttaatgcagctacgtccaagca |
| | gtctgccaaatcatccaagtcaacatcaagcaaaaaagcaggcagtgttggaaagccttcacct |
| | cgtcgtcgttcagttggaatagatcaagatgccagctaatagcagctccaattacgaaagaaaa |
| | atattttcaactagaatcgagcttaataaattgtccaattaagtatgctcaacagcagttcttaaagc |
| | tcttcttcaattaaatttataaaggtatggctatggcgactgaagtaccttttacttttagcacgtggg |
| | atttatctaaaccccttaccccagctcgtagttctctctttagcttgaaaccgattggggttggaac |
| | ggctgcagtagaaagcctcactggatatattgcacgtttggctgaagctcactgtatctggccag |
| | gagatcttcttgcagctgaatttagttcttcatattcctattcttacctgcatagcatgaggggccgt |
| | attgctgctttgaatggtaatagccaaatggctcaggatgctgtacttaagcttcaagagcttacct |
| | tacaggagaatttatccgctttaacaatgttaggctgggctaatattttacccgcaaaagggttgtt |
| | aaatcggactcgaacttggtgtccggtttgttttgaggcgtggcgtttcatcaaaagtcctatatat |
| | gagcctctattatggtatttaacctcggtcaaatattgtccaattcatcagcaacgccttttatctcaa |
| | tgtccattctgcgggtgccagcagcctcctctagactggaaaactcggccgggctattgttcaa |
| | ggtgccaggtgtggctgggttcaccaccaggtgtaacttccagtaattcgctttctaatacagaa |
| | ataaggtgggagagttgggttgcccagagctgcggaaatttgttagcctatagtcctcgtttaca |
| | caagatatccgttagcgttcaggtcgcagcatccctttctaaatgtatcaacctttatgcagaggg |
| | caatattgcggagtttgcccgtcaaatgagcttgccaaaaacttctgtttggctgtggcagtctgg |
| | tcaggtactccccgagttatccaggcttttaagcatctgctatcacttcaatctgtcgctcttagact |
| | ttctaattaatacaccagttcttaagataaggagagacttaattccagaaaagagaactaatgagtt |
| | tcgccagcctcgaagagatccctcaaccatatctcaattgcaacctattctgggattaattcttttc |
| | gagcagccacctccctctttagcagcagtagcgtgtcgcttaggttgtagctcaagatccctgcg |
| | ccataattttcctgatctctgtggctctatttctgcacgttaccttcaacaccgtgatgaagtttataa |
| | agaaaaattgaatcaattctgtcaagacttgcgccaagttgtacttcaactttattcggcaggcatt |
| | gatccgactcggagccaagttgctcgccgtctgaggaaacccgcttattttagagaacccgaag |
| | tcgaggcaatgcttgagtctgttcgacgagaattaggcttgcacagcaattctcagcatgagact |
| | aattctcacaaaaacgcccagttttaacttaagtcgaacagaaagtaatcgtattgtacacggcc |
| | gcataatcgaaattaatacgactcactataggggaattgtgagcggataacaattccccatcttag |
| | tatattagttaagtataagaaggagatatacatatggcagatctcaattggatatcggccggccac |
| | gcgatcgctgacgtcggtaccctcgagtctggtaaagaaaccgctgctgcgaaatttgaacgc |
| | cagcacatggactcgtctactagcgcagcttaattaacctaggctgctgccaccgctgagcaat |
| | aactagcataaccccttggggcctctaaacgggtcttgaggggttttttgctgaaacctcaggca |
| | tttgagaagcacacggtcacactgcttccggtagtgccctgaaccgacgaccgggtcatcgtg |
| | gccggatcttgcggcccctcggcttgaacgaattgttagacattatttgccgactaccttggtgat |
| | ctcgcctttcacgtagtggacaaattcttccaactgatctgcgcgcgaggccaagcgatcttcttc |
| | ttgtccaagataagcctgtctagcttcaagtatgacgggctgatactgggccggcaggcgctcc |
| | attgcccagtcggcagcgacatccttcggcgcgattttgccggttactgcgctgtaccaaatgc |
| | gggacaacgtaagcactacatttcgctcatcgccagcccagtcgggcggcgagttccatagcg |
| | ttaaggtttcatttagcgcctcaaatagatcctgttcaggaaccggatcaaagagttcctccgccg |
| | ctggacctaccaaggcaacgctatgttctcttgcttttgtcagcaagatagccagatcaatgtcga |
| | tcgtggctggctcgaagatacctgcaagaatgtcattgcgctgccattctccaaattgcagttcgc |
| | gcttagctggataacgccacggaatgatgtcgtcgtgcacaacaatggtgacttctacagcgcg |
| | gagaatctcgctctctccaggggaagccgaagtttccaaaaggtcgttgatcaaagctcgccgc |
| | gttgtttcatcaagccttacggtcaccgtaaccagcaaatcaatatcactgtgtggcttcaggccg |
| | ccatccactgcggagccgtacaaatgtacggccagcaacgtcggttcgagatggcgctcgatg |
| | acgccaactacctctgatagttgagtcgatacttcggcgatcaccgcttccctcatactcttcctttt |
| | tcaatattattgaagcatttatcagggttattgtctcatgagcggatacatatttgaatgtatttagaa |
| | aaataaacaaatagctagctcactcggtcgctacgctccgggcgtgagactgcggcgggcgct |
| | gcggacacatacaaagttacccacagattccgtggataagcaggggactaacatgtgaggca |
| | aaacagcagggccgcgccggtggcgtttttccataggctccgccctcctgccagagttcacata |
| | aacagacgcttttccggtgcatctgtgggagccgtgaggctcaaccatgaatctgacagtacgg |
| | gcgaaacccgacaggacttaaagatccccaccgtttccggcgggtcgctccctcttgcgctctc |
| | ctgttccgaccctgccgtttaccggatacctgttccgcctttctcccttacgggaagtgtggcgctt |
| | tctcatagctcacacactggtatctcggctcggtgtaggtcgttcgctccaagctgggctgtaag |
| | caagaactccccgttcagcccgactgctgcgccttatccggtaactgttcacttgagtccaaccc |
| | ggaaaagcacggtaaaacgccactggcagcagccattggtaactgggagttcgcagaggatt |
| | tgtttagctaaacacgcggttgctcttgaagtgtgcgccaaagtccggctacactggaaggaca |
| | gatttggttgctgtgctctgcgaaagccagttaccacggttaagcagttccccaactgacttaacc |
| | ttcgatcaaaccacctccccaggtggttttttcgtttacagggcaaaagattacgcgcagaaaaa |
| | aaggatctcaagaagatcctttgatcttttctactgaaccgctctagatttcagtgcaatttatctctt |
| | caaatgtagcacctgaagtcagccccatacgatataagttgtaattctcatgttagtcatgccccg |
| | cgcccaccggaaggagctgactgggttgaaggctctcaagggcatcggtcgagatcccggtg |
| | cctaatgagtgagctaacttacattaattgcgttgcgctcactgcccgctttccagtcgggaaacc |
| | tgtcgtgccagctgcattaatgaatcggccaacgcgcggggagaggcggtttgcgtattgggc |
| | gccagggtggtttttcttttcaccagtgagacgggcaacagctgattgcccttcaccgcctggcc |
| | ctgagagagttgcagcaagcggtccacgctggtttgccccagcaggcgaaaatcctgtttgat |
| | ggtggttaacggcgggatataacatgagctgtcttcggtatcgtcgtatcccactaccgagatgt |
| | ccgcaccaacgcgcagcccggactcggtaatggcgcgcattgcgcccagcgccatctgatcg |
| | ttggcaaccagcatcgcagtgggaacgatgccctcattcagcatttgcatggtttgttgaaaacc |
| | ggacatggcactccagtcgccttcccgttccgctatcggctgaatttgattgcgagtgagatattt |
| | atgccagccagccagacgcagacgcgccgagacagaacttaatgggcccgctaacagcgc |
| | gatttgctggtgacccaatgcgaccagatgctccacgcccagtcgcgtaccgtcttcatgggag |
| | aaaataatactgttgatgggtgtctggtcagagacatcaagaaataacgccggaacattagtgc |
| | aggcagcttccacagcaatggcatcctggtcatccagcggatagttaatgatcagcccactgac |
| | gcgttgcgcgagaagattgtgcaccgccgctttacaggcttcgacgccgcttcgttctaccatc |
| | gacaccaccacgctggcacccagttgatcggcgcgagatttaatcgccgcgacaatttgcgac |
| | ggcgcgtgcagggccagactggaggtggcaacgccaatcagcaacgactgtttgcccgcca |
| | gttgttgtgccacgcggttgggaatgtaattcagctccgccatcgccgcttccactttttcccgcg |
| | ttttcgcagaaacgtggctggcctggttcaccacgcgggaaacggtctgataagagacaccgg |
| | catactctgcgacatcgtataacgttactggtttcacattcaccaccctgaattgactctcttccgg |
| | gcgctatcatgccataccgcgaaaggttttgcgccattcgatggtgtccgggatctcgacgctct |
| | cccttatgcgactcctgcattaggaaattaatacgactcactata (SEQ ID NO: 92) |
| pHelper(CyCAST)_ | ggggaattgtgagcggataacaattcccctgtagaaataattttgtttaactttaataaggagatat |
| Shuffled-spacer_ | accatggtttccaagcctgaatcccggtctacgggactgaaacaccatctatgtatagctacgat |
| ΔTniQ | ggaatagtaaagggtttccaagcctgaatcccggtctacgggactgaaacgatccgaattcga |
| | gctcggcgcgcctgcaggtcgacaagcttgcggccgcataatgcttaagtcgaacagaaagta |
| | atcgtattgtacacggccgcataatcgaaattaatacgactcactataggggaattgtgagcgga |
| | taacaattccccatcttagtatattagttaagtataagaaggagatatacatatggctaccctactcc |
| | aaaccctcctgttacgaacgttgtcacccgaggcggatccagttctctatgtctacattgagacg |
| | gtactacctgcaatggaacgggaatttggcttgattccagcccttggtggctcggaggaagcac |
| | actttcatctgctgaggcgacggggaaacaaatatgcggcggaaaattccaagcggtacgcca |
| | gtcgctctgatcaaagcctgctggttcatgtgctcaatgccttgctgaccgcctggaatttgttgc |
| | cattcctaagctccacatttgaactatcagatgatgagaaacggctgctctgcctggggctgacc |
| | ctacacgattacaacaagtggtgccagggtgaggaggaagattcgccgattgcatgcgaagta |
| | gcagagattcttgagctttgtcaaacattgggagagcggttgaactttgacggattctgggcaga |
| | gtggcgggagtatttgacagagattgcctaccttgcccaaaacacccagggaaaagttggaac |
| | aaatctacccaaagcgaattggggcagcttcaaaattctagatacccgtcgtctggattcgcccc |
| | tacgatggctactcgcttttggcgatgtcgcagtccatctcagcgatccatctgacatcgcaatga |
| | aaacggggggagatcgcctgcgggaacatctgcgctttctaggtattcgtcgcaagctggtcta |
| | ccatcgcctgcgcaactgtacagggctattaaccaacggaattcataatgcggtaatgcattttg |
| | ctagagaactggattggcaacccatcctcttctttgctcagggtgttgtttatctcgctcctcaggg |
| | tgctggcgttccggatcgcttagaacttcaggaatttctctgggagcaggtcagcaaccgattag |
| | ccaccagcatgttgcggggagaggttggtttcaaacgggatggcaagggactcaaggtggct |
| | ccccaaaccctggaactgttctcccctgctcagttgattcggaacttgcccggtgttgtccaatctt |
| | atgtcggaaacgctaaatctcctgcgactccaaaacgacttgagaagctggaactcagttcaac |
| | agagcgagagtttttggctaaaggtgcagatttaagagcagatcgcattgccgagtttatctttct |
| | ggcgcaaaaagagtttttcggagataaaccagaatttatcacttggatgcttgaaactctggggg |
| | ttcaaactgctatcacttcggagcagacgcagcttcaatccggtggtgtgaactggggatggtat |
| | cacgcagcagcctattacgtcgcgagcaatgccacgctggatttggaagagatttcggagaaa |
| | cttcaggaatttgccgagcagttggcaacctgggcggaggaaaacaatttcttcccagagcatc |
| | gcagcccaacccatgacatctttttcaactatctgactcaatacctggaagttcaggggtgggag |
| | cagcaggagcagtcctttcagcttgaactggcaggctatgttggcgcaaagactaaaactgcca |
| | gacaaccgatttgctccctcagttccggtgaatttgcttcggaagaccagatggattctgttgtcct |
| | gttcaaaccgcagcagtacagcaacaagaatcctctcggtggacggcagatcaagcggggg |
| | atttccaaaatctggtccctggaaatgctgctcaggcaggctctttggtctgcgccagcaggcaa |
| | actagaggaacaacaacccattttcctttacatcttcccggcatacgtttactcgccccaggttgct |
| | gctgctgtccggttactggtgaatgatctgaagcgtgtcaacctctgggaagtccgtaaacactg |
| | gttggagaatggcatggatgctcaggcgttgcgatcgtttccctggctagtcggtgacaaaaac |
| | gattcgggtaactctgctccatccagatactctggtgaagacttgcctttcatggcaatgaactac |
| | accaccaccagaggcaaaacgataactgatgcctgggttgaacctgctttcctgacactggtgc |
| | tgcctttgttgctgggtgtgaaagttgtcgccaccagtagccagattccactctatgaaagcgac |
| | aacgacttttgcgaatctgtcaagctggatggacccgccgatttctgggctttgctgcggctacc |
| | aacttccctgcatctccaggattggattggggggcgtgttcaaaatttagcagaaggcttgaacc |
| | gcttgatggttgcctactgcctgcatctggacagtcgtagcgctccaccagatgcgcgctggag |
| | agccttaaacggcagtgtccgagagatgacaacaaatgttctaaacattttttcacttgcagagg |
| | agggattacgtcgtgatggctctaaacgcgatgcaacagttgaagaaattaaccgctattggaa |
| | atacgcccagctgtgggcagagggagactctgatatgcaaaacaagttgaagattaccaaacg |
| | cttagtcgatgagtaccgccagttttacagagccagtgcgaacgaatccagccatacggttttgc |
| | tgccattttctaaggcactggaacacattctttcggtaccaaatgattgggacgatgaggaaatta |
| | ttgttcaaggggcaggacagttacaggccgcgttagatcggcaggaagtctacaaacgtcccc |
| | tcatgcttgacaaagagattccctttgaaattcgtcggatgaaggagtttgaggcaattcaaacct |
| | tcatgaccacctgcgtcattgacctatttggacaaatgtgtaaaggcgatcgcgcgctcctgcag |
| | gaacatcgcaaccgccttaaatcaggggtcgaatttgcctatcgtcagttagccctgcaagaga |
| | aacaagccaaagccaaccaaaaatctggagaacaatcatgagcattcttgaaaccttaaagcc |
| | agagttccacacagccttccctcgcctcgcctctggcaaatatgtccatttcttcatggtgcgtca |
| | cagccagtcgttccccgtctttcaaaccgatggcgttttgaacactgcccgtactcaggctgggtt |
| | aaatgcaggcgatgaaaaatccagacaggcaatcagtcgcctggtcatgttcaaacggaaaca |
| | ggtgacaccagaacgactcacaggacgggaactgctgcgatcgctgggcattaacggagtag |
| | ttgaaggggaagccaatttctgtgaatacaacggcgaaagatcctgtaaaaaatgccctgattg |
| | cattctctatggttttgcgattggggacagtggctcggaacgttccaaagtctactctgattcagc |
| | cttttccctgagtccttatgaagtttcccatcgcagttttaccttcaatgctccctttgaaggtggaac |
| | catgagtgagaagggtgaaatgcggagtgcgatcaacgaactggatcatgttttgccggaagtt |
| | gcttttccttgcgttgaaacactacgtgaccccacgctggaaggcttcatttatgtaatgggcaac |
| | ctgatccggactcggcgctatggagcgcaggagtcgcgtacagggacaatggcgaatcacat |
| | tattggcgttaccctatgtgatggcgagatcttcagcaatttgcactttacccaagccctctacgat |
| | gccctgaagggtgatttgagtgcgccaattagtgatttatgcagagaggcggcgatcgtggca |
| | gaaaacttgctaaatcaggaacctgtgcggaagatccaagttatttttggagagcagttgaacga |
| | actcctggctgaggtagcgggaatctgccaggatgaaactcgcttgcgaaacactgtgacagc |
| | cttgtaccagcaatcacaagcctatgccgagacctatggtgctgctgctaagaaaggcaaagg |
| | caaatcttaaggtggctgccaatggttatttatcgatgcaatctcacgttgcacgacaacgtattct |
| | tcgccactcgcgagatggggattctctacgaaacagagaagtatctacacaactgggcactca |
| | gctttgctctgtttgaagccacccacatccccaaaccctatcgcttgcagggtgcgatcgctcaa |
| | cgacccggttatctggaagctgacaacgagcaaaaccttttagttctgaatcacgcaggcatcta |
| | tgttttccctgctctaccacttcagtggtcctaccaagtcaataccttcaaagcggctcaagtgcct |
| | tactatggcaaatccaaacagttcggggataagggagctgatcgcaactacccgattaactacg |
| | gacgggcaaaagaactggcagttggcagcacctaccgaacgtatattcttgcccctgaaccaa |
| | tccagatcccccactggatacggttgggtaagtgggcagccaagattcaggtggacgcaaga |
| | cccatacctaactgtggaatccgacaacggtcaggagaatacacctgcgaccatccactaaatc |
| | cgatcgacctgtccggttcaacccgattgctgttatacaaccgcattgtcatgccacccgctagtc |
| | tcgtcagtcagtcgcgagtttctggggactactgggaaatttctaaaccatccaattggactgaat |
| | ttccggactggtcagacctgaaagacatacccaatcaagtttgcttacccagcggagtagcctat |
| | ggagccagtactgccattgccgcatcttaagtccaccgctctgcatatcctggtaattcatttagg |
| | cgttgccgaaagcggagccttgcctgttgaatccggtcgagccgtccatgctcaagttttggact |
| | ggttccgcatggcggatccggcaatttctgaggcaattcacaaaagccaggaatccccgctca |
| | gtctctcaggcctggtcgggaaacgccgtaaagtaggggtgcgggcaggagatgaattttact |
| | tccgaattgggttgttagatgggagtttgattgaaccactgctgagtggtttggagaagtggggc |
| | gacaagcccttttctctggcaaagtgtccctttgttttccgcagtatcaatatgttgccaggaactg |
| | acccctgggttggttcttccgattatgccttgcttacccaaaccccggctactttggataacctagc |
| | tctcaagctcctgtcgccaacaagcttcaagtcgaatgccgggcaaggaatccaaccctttcctt |
| | tgccagagtcagttttcggaaatttacatcgtcgctggaatgtctttgcaccaaaggaattgcagtt |
| | tccaaaaattcaatggagtgggctggtctctgattatgacctcaaaactcaaaagttaaggatgg |
| | aaaactcggttgagatcggcgcagtgggctgggtgcgctatcggtttccagatccagaacaag |
| | cgagaattgcaaccattctggctcactttgccttttttgctggagttgggcgtaagacagcaatgg |
| | ggatgggacaggtggtgttggacaaaaaaacgtagcctaggctgctgccaccgctgagcaat |
| | aactagcataaccccttggggcctctaaacgggtcttgaggggttttttgctgaaacctcaggca |
| | tttgagaagcacacggtcacactgcttccggtagtcaataaaccggtaaaccagcaatagacat |
| | aagcggctatttaacgaccctcgggatctcgacgctctcccttatgcgactcctgcattaggaaa |
| | ttaatacgactcactataggggaattgtgagcggataacaattcccctgtagaaataattttgttta |
| | actttaataaggagatataccatgcttgatgaaaaaagcttcgacaactggtgccaatccgttggt |
| | ctgagccaggcagcacgtgaagttgtagagcgtgtgcgatcctcttcaccctcccgtcgcgtac |
| | agagcggtagaggaaacgtctccggcaattaccctagccgcaaaatgggcgtcacaattcagt |
| | ttgagtcccatcgaaatgagcttgcccgcatctacgaactggagcatgaccccggcgttctgga |
| | atattacgatcaacctcccaaaatcgaacttgattatttgtcaaaaaatgggaggcgtgttcgtact |
| | caatacacacctgatttctttgttatccgtaatgactcagcaggttgggaggagtgtaaaacagag |
| | gatgaccttaagcaattagctgaggacagccctaaccgttaccaacgagctgaggacaatcgc |
| | tggcgatgtccacctgcagaagcctatgctcttccccttgagttgtattttcaggtacggtctagtg |
| | ctgaaattaactggattttccagcaaaactttgtatggctagaagactaccttggtactgaatcactt |
| | actatggatgagtcggttgcagatgcaatcatagccacagtcagagcaaacgagggtatcaca |
| | ctactcgaactgattcaacaattacaaggcacagatgctgatgctatctacatcctgattgcaacc |
| | gaacaaatttatgttgatttgatggctgaacggttagcgaacagcgatcgggcacgggtattttc |
| | cagtccagaaattgctcaagcctttcaactcattacgctggtcgaagcacctctggctaacagttc |
| | tctccatatagttcaggtgggcgttggcacaaccatacactgggatggtgaagtctgggagatt |
| | gttaatacaggctctaccatgacaggattgttgcgatcggacggtaagtttatcgagttgcccaat |
| | tctgcaattgatgaattgattcagaagggcaagattcaatcaatttcagcacaacaagcccctgat |
| | agcaaagcagaagttgaagaaatgcttcgctgtgctcgtccagccgacattaaagaagccaac |
| | cgtcgctacgaaatcattcagccttacctggaaaaggatgcaccagcttgtccaactcgaacaat |
| | tcggcgctggcgtaatcagtattggcaggcagaaaagctttacgggaatggctatgttggtttac |
| | taccaaatcatcagaagaaagggaattatttagttaaggttaatgaagtaactcaagcttttatgctt |
| | gagtttattgagaagcattacgaaaatctcaaacagcgtagtaagcttagagtttatcaggcttttg |
| | tggaggcatgtgaaagccacgaaccgaaactaaaccctccaagccgtactacattcacactag |
| | cgattcagaaacgagcaggacatcagcaaaccttgaaacgtgaaggacgaagagctgccatt |
| | cagaaggaggagttttactgggaactggagctaactactcctcgacacggcgatcgccccttc |
| | gagatcgttcatattgatcacactcaactcgatattgagcttatcagttctttgaactccctggccca |
| | atgcaaccctacatctgttgatgccacttacaatttgggtcgtccttgggcaacgttcatggttgat |
| | gcttacagccgccgtttgctagcagtttacataacctatgaagaacccagctatcgttcctgcatg |
| | atggtgctccgtatttgcgtacagcgatttaagcgttttcctcagacgatcgtggtagataatggtg |
| | cagaatttcacagccattactttgaacaactactggcaacatatcaatgcacaaagaagcacag |
| | gccacctgcaaaagctcgctttggtgcagttgttgaacgattatttggcactgcaaataagcaatt |
| | aattcatgagttgcagggaaatacccaaattactcgcaagcatcgccaagttactcaatctgttca |
| | tccccaaaaattggctctttggacgttaggcgatctctacgaatcactatgtgaatgggcttacaa |
| | agtttatgctgaccgccagcaccctgcgctagggcaaagccctaatgctgcattcgctcaaggg |
| | ctggcgttgggaggtagtcgcatccatcgccgagtagagtatgaccaagtcttccgtatcttgac |
| | attgcccgcaccggaaagaggacaacgcaaagttcagccaggacaagggataaaacttagc |
| | aatatctactactggtcaaatgcctttcgagacccagaaattgaacaatcgatggttgaagttcgtt |
| | atgatccctttgatgccagcattgcctatgcactggtgcgaaaacaatgggtccggtgtatttcct |
| | cctactatcaacagtttcaaaatcgttcagaaaaggaaattcgtttaataactgctgagttgattaa |
| | gcaaaagcgtgaccagggaagagatacaaatgttacggataaagaattggcgcagtttttaag |
| | ctctacagaaatgaaagaaggcaaattcttggagcaacgcttgcggactacagaaaacagggt |
| | agttattcagaatagtacagctacagaagacgatccccccatcactaattctcagaattcttacga |
| | gccatcgcttatgctgccaagtgaggatgagttggatgatcaggaagactggttaaataccggc |
| | aaagctgaggcagaattagaattctatggagagtactagcgatgcaacctcaaataagcttccc |
| | aaaagagttactgcttcaaccaattcctgatcgaattgcctatttcaaagattacacaatggcacat |
| | ccgaagcttatagaagcagaccggaagttaatgcatgctattcgtgaacctggaggagctgctc |
| | ttatctttgtctttggtcctaccggagttggaaagtctactctattgaaacgaattactcaaaagctg |
| | cttaaagaagcattgtctttgatggagactgacaaaggctatattcctattgcgggggtagagata |
| | aaaactccagaggatagtaactttgattggaacgatttttatgttcgtgctcttatggcacttaatga |
| | gccaatgatcgagaaaaaaatcaaccgtacaggtgcaaaactaaaattacggttggcacttgaa |
| | tctgcccttcgtaatcgccgtcctgatgctttttacatagacgaagctcaaaattttggtaaggtag |
| | ccagtgggcgaaaattgcaagatcaaacagattgcattaaatctcttgcaaatctcgctcaaact |
| | cggtttatcttatgtgggatttatgaacttttaccactacgaaatctaagtgggcagctatgccgcc |
| | gtagccatagtattcactttccacgttattcggcagattctgagcaggatctaaaggcctttaaaag |
| | tgcgttattaacactacaaaatcaactccctcttcccgaaccacctgatttagtggcgcattggga |
| | attttgttatgagagaactatcggctgtattggtattttgaaagatttgctccttcggactctaagcg |
| | cgattttagtacgggatggagacaaagttttgacattgaagcccaaggatttagaacaacgtgca |
| | tggtctttagaagaatgtttggttatgctgagagaggcaaaggaaggagagcaacaacttactg |
| | acaaatcgggcttacatgaggaattacggacagctttagggtttaatgcagctacgtccaagca |
| | gtctgccaaatcatccaagtcaacatcaagcaaaaaagcaggcagtgttggaaagccttcacct |
| | cgtcgtcgttcagttggaatagatcaagatgccagctaatagcagctccaattacgaaagaaaa |
| | atattttcaactagaatcgagcttaataaattgtccaattaagtatgctcaacagcagttcttaaagc |
| | tcttcttcaattaaatttataaaggtatggctatggcgactgaagtaccttttacttttagcacgtggg |
| | atttatctaaaccccttaccccagctcgtagttctctctttagcttgaaaccgattggggttggaac |
| | ggctgcagtagaaagcctcactggatatattgcacgtttggctgaagctcactgtatctggccag |
| | gagatcttcttgcagctgaatttagttcttcatattcctattcttacctgcatagcatgaggggccgt |
| | attgctgctttgaatggtaatagccaaatggctcaggatgctgtacttaagcttcaagagcttacct |
| | tacaggagaatttatccgctttaacaatgttaggctgggctaatattttacccgcaaaagggttgtt |
| | aaatcggactcgaacttggtgtccggtttgttttgaggcgtggcgtttcatcaaaagtcctatatat |
| | gagcctctattatggtatttaacctcggtcaaatattgtccaattcatcagcaacgccttttatctcaa |
| | tgtccattctgcgggtgccagcagcctcctctagactggaaaactcggccgggctattgttcaa |
| | ggtgccaggtgtggctgggttcaccaccaggtgtaacttccagtaattcgctttctaatacagaa |
| | ataaggtgggagagttgggttgcccagagctgcggaaatttgttagcctatagtcctcgtttaca |
| | caagatatccgttagcgttcaggtcgcagcatccctttctaaatgtatcaacctttatgcagaggg |
| | caatattgcggagtttgcccgtcaaatgagcttgccaaaaacttctgtttggctgtggcagtctgg |
| | tcaggtactccccgagttatccaggcttttaagcatctgctatcacttcaatctgtcgctcttagact |
| | ttctaattaatacaccagttcttaagataaggagagacttaattccagaaaagagaactaatgagtt |
| | tcgccagcctcgaagagatccctcaaccatatctcaattgcaacctattctgggattaattcttttc |
| | gagcagccacctccctctttagcagcagtagcgtgtcgcttaggttgtagctcaagatccctgcg |
| | ccataattttcctgatctctgtggctctatttctgcacgttaccttcaacaccgtgatgaagtttataa |
| | agaaaaattgaatcaattctgtcaagacttgcgccaagttgtacttcaactttattcggcaggcatt |
| | gatccgactcggagccaagttgctcgccgtctgaggaaacccgcttattttagagaacccgaag |
| | tcgaggcaatgcttgagtctgttcgacgagaattaggcttgcacagcaattctcagcatgagact |
| | aattctcacaaaaacgcccagttttaacttaagtcgaacagaaagtaatcgtattgtacacggcc |
| | gcataatcgaaattaatacgactcactataggggaattgtgagcggataacaattccccatcttag |
| | tatattagttaagtataagaaggagatatacatatggcagatctcaattggatatcggccggccac |
| | gcgatcgctgacgtcggtaccctcgagtctggtaaagaaaccgctgctgcgaaatttgaacgc |
| | cagcacatggactcgtctactagcgcagcttaattaacctaggctgctgccaccgctgagcaat |
| | aactagcataaccccttggggcctctaaacgggtcttgaggggttttttgctgaaacctcaggca |
| | tttgagaagcacacggtcacactgcttccggtagtgccctgaaccgacgaccgggtcatcgtg |
| | gccggatcttgcggcccctcggcttgaacgaattgttagacattatttgccgactaccttggtgat |
| | ctcgcctttcacgtagtggacaaattcttccaactgatctgcgcgcgaggccaagcgatcttcttc |
| | ttgtccaagataagcctgtctagcttcaagtatgacgggctgatactgggccggcaggcgctcc |
| | attgcccagtcggcagcgacatccttcggcgcgattttgccggttactgcgctgtaccaaatgc |
| | gggacaacgtaagcactacatttcgctcatcgccagcccagtcgggcggcgagttccatagcg |
| | ttaaggtttcatttagcgcctcaaatagatcctgttcaggaaccggatcaaagagttcctccgccg |
| | ctggacctaccaaggcaacgctatgttctcttgcttttgtcagcaagatagccagatcaatgtcga |
| | tcgtggctggctcgaagatacctgcaagaatgtcattgcgctgccattctccaaattgcagttcgc |
| | gcttagctggataacgccacggaatgatgtcgtcgtgcacaacaatggtgacttctacagcgcg |
| | gagaatctcgctctctccaggggaagccgaagtttccaaaaggtcgttgatcaaagctcgccgc |
| | gttgtttcatcaagccttacggtcaccgtaaccagcaaatcaatatcactgtgtggcttcaggccg |
| | ccatccactgcggagccgtacaaatgtacggccagcaacgtcggttcgagatggcgctcgatg |
| | acgccaactacctctgatagttgagtcgatacttcggcgatcaccgcttccctcatactcttcctttt |
| | tcaatattattgaagcatttatcagggttattgtctcatgagcggatacatatttgaatgtatttagaa |
| | aaataaacaaatagctagctcactcggtcgctacgctccgggcgtgagactgcggcgggcgct |
| | gcggacacatacaaagttacccacagattccgtggataagcaggggactaacatgtgaggca |
| | aaacagcagggccgcgccggtggcgtttttccataggctccgccctcctgccagagttcacata |
| | aacagacgcttttccggtgcatctgtgggagccgtgaggctcaaccatgaatctgacagtacgg |
| | gcgaaacccgacaggacttaaagatccccaccgtttccggcgggtcgctccctcttgcgctctc |
| | ctgttccgaccctgccgtttaccggatacctgttccgcctttctcccttacgggaagtgtggcgctt |
| | tctcatagctcacacactggtatctcggctcggtgtaggtcgttcgctccaagctgggctgtaag |
| | caagaactccccgttcagcccgactgctgcgccttatccggtaactgttcacttgagtccaaccc |
| | ggaaaagcacggtaaaacgccactggcagcagccattggtaactgggagttcgcagaggatt |
| | tgtttagctaaacacgcggttgctcttgaagtgtgcgccaaagtccggctacactggaaggaca |
| | gatttggttgctgtgctctgcgaaagccagttaccacggttaagcagttccccaactgacttaacc |
| | ttcgatcaaaccacctccccaggtggttttttcgtttacagggcaaaagattacgcgcagaaaaa |
| | aaggatctcaagaagatcctttgatcttttctactgaaccgctctagatttcagtgcaatttatctctt |
| | caaatgtagcacctgaagtcagccccatacgatataagttgtaattctcatgttagtcatgccccg |
| | cgcccaccggaaggagctgactgggttgaaggctctcaagggcatcggtcgagatcccggtg |
| | cctaatgagtgagctaacttacattaattgcgttgcgctcactgcccgctttccagtcgggaaacc |
| | tgtcgtgccagctgcattaatgaatcggccaacgcgcggggagaggcggtttgcgtattgggc |
| | gccagggtggtttttcttttcaccagtgagacgggcaacagctgattgcccttcaccgcctggcc |
| | ctgagagagttgcagcaagcggtccacgctggtttgccccagcaggcgaaaatcctgtttgat |
| | ggtggttaacggcgggatataacatgagctgtcttcggtatcgtcgtatcccactaccgagatgt |
| | ccgcaccaacgcgcagcccggactcggtaatggcgcgcattgcgcccagcgccatctgatcg |
| | ttggcaaccagcatcgcagtgggaacgatgccctcattcagcatttgcatggtttgttgaaaacc |
| | ggacatggcactccagtcgccttcccgttccgctatcggctgaatttgattgcgagtgagatattt |
| | atgccagccagccagacgcagacgcgccgagacagaacttaatgggcccgctaacagcgc |
| | gatttgctggtgacccaatgcgaccagatgctccacgcccagtcgcgtaccgtcttcatgggag |
| | aaaataatactgttgatgggtgtctggtcagagacatcaagaaataacgccggaacattagtgc |
| | aggcagcttccacagcaatggcatcctggtcatccagcggatagttaatgatcagcccactgac |
| | gcgttgcgcgagaagattgtgcaccgccgctttacaggcttcgacgccgcttcgttctaccatc |
| | gacaccaccacgctggcacccagttgatcggcgcgagatttaatcgccgcgacaatttgcgac |
| | ggcgcgtgcagggccagactggaggtggcaacgccaatcagcaacgactgtttgcccgcca |
| | gttgttgtgccacgcggttgggaatgtaattcagctccgccatcgccgcttccactttttcccgcg |
| | ttttcgcagaaacgtggctggcctggttcaccacgcgggaaacggtctgataagagacaccgg |
| | catactctgcgacatcgtataacgttactggtttcacattcaccaccctgaattgactctcttccgg |
| | gcgctatcatgccataccgcgaaaggttttgcgccattcgatggtgtccgggatctcgacgctct |
| | cccttatgcgactcctgcattaggaaattaatacgactcactata (SEQ ID NO: 93) |
| pHelper(CyCAST)_PSP | ggggaattgtgagcggataacaattcccctgtagaaataattttgtttaactttaataaggagatat |
| 1_ΔTnsD | accatggtttccaagcctgaatcccggtctacgggactgaaacgagaagtcatttaataaggcc |
| | actgttaaacgtaggtttccaagcctgaatcccggtctacgggactgaaacgatccgaattcga |
| | gctcggcgcgcctgcaggtcgacaagcttgcggccgcataatgcttaagtcgaacagaaagta |
| | atcgtattgtacacggccgcataatcgaaattaatacgactcactataggggaattgtgagcgga |
| | taacaattccccatcttagtatattagttaagtataagaaggagatatacatatggctaccctactcc |
| | aaaccctcctgttacgaacgttgtcacccgaggcggatccagttctctatgtctacattgagacg |
| | gtactacctgcaatggaacgggaatttggcttgattccagcccttggtggctcggaggaagcac |
| | actttcatctgctgaggcgacggggaaacaaatatgcggcggaaaattccaagcggtacgcca |
| | gtcgctctgatcaaagcctgctggttcatgtgctcaatgccttgctgaccgcctggaatttgttgc |
| | cattcctaagctccacatttgaactatcagatgatgagaaacggctgctctgcctggggctgacc |
| | ctacacgattacaacaagtggtgccagggtgaggaggaagattcgccgattgcatgcgaagta |
| | gcagagattcttgagctttgtcaaacattgggagagcggttgaactttgacggattctgggcaga |
| | gtgggggagtatttgacagagattgcctaccttgcccaaaacacccagggaaaagttggaac |
| | aaatctacccaaagcgaattggggcagcttcaaaattctagatacccgtcgtctggattcgcccc |
| | tacgatggctactcgcttttggcgatgtcgcagtccatctcagcgatccatctgacatcgcaatga |
| | aaacggggggagatcgcctgcgggaacatctgcgctttctaggtattcgtcgcaagctggtcta |
| | ccatcgcctgcgcaactgtacagggctattaaccaacggaattcataatgcggtaatgcattttg |
| | ctagagaactggattggcaacccatcctcttctttgctcagggtgttgtttatctcgctcctcaggg |
| | tgctggcgttccggatcgcttagaacttcaggaatttctctgggagcaggtcagcaaccgattag |
| | ccaccagcatgttgcggggagaggttggtttcaaacgggatggcaagggactcaaggtggct |
| | ccccaaaccctggaactgttctcccctgctcagttgattcggaacttgcccggtgttgtccaatctt |
| | atgtcggaaacgctaaatctcctgcgactccaaaacgacttgagaagctggaactcagttcaac |
| | agagcgagagtttttggctaaaggtgcagatttaagagcagatcgcattgccgagtttatctttct |
| | ggcgcaaaaagagtttttcggagataaaccagaatttatcacttggatgcttgaaactctggggg |
| | ttcaaactgctatcacttcggagcagacgcagcttcaatccggtggtgtgaactggggatggtat |
| | cacgcagcagcctattacgtcgcgagcaatgccacgctggatttggaagagatttcggagaaa |
| | cttcaggaatttgccgagcagttggcaacctgggggaggaaaacaatttcttcccagagcatc |
| | gcagcccaacccatgacatctttttcaactatctgactcaatacctggaagttcaggggtgggag |
| | cagcaggagcagtcctttcagcttgaactggcaggctatgttggcgcaaagactaaaactgcca |
| | gacaaccgatttgctccctcagttccggtgaatttgcttcggaagaccagatggattctgttgtcct |
| | gttcaaaccgcagcagtacagcaacaagaatcctctcggtggacggcagatcaagcggggg |
| | atttccaaaatctggtccctggaaatgctgctcaggcaggctctttggtctgcgccagcaggcaa |
| | actagaggaacaacaacccattttcctttacatcttcccggcatacgtttactcgccccaggttgct |
| | gctgctgtccggttactggtgaatgatctgaagcgtgtcaacctctgggaagtccgtaaacactg |
| | gttggagaatggcatggatgctcaggcgttgcgatcgtttccctggctagtcggtgacaaaaac |
| | gattcgggtaactctgctccatccagatactctggtgaagacttgcctttcatggcaatgaactac |
| | accaccaccagaggcaaaacgataactgatgcctgggttgaacctgctttcctgacactggtgc |
| | tgcctttgttgctgggtgtgaaagttgtcgccaccagtagccagattccactctatgaaagcgac |
| | aacgacttttgcgaatctgtcaagctggatggacccgccgatttctgggctttgctgcggctacc |
| | aacttccctgcatctccaggattggattggggggcgtgttcaaaatttagcagaaggcttgaacc |
| | gcttgatggttgcctactgcctgcatctggacagtcgtagcgctccaccagatgcgcgctggag |
| | agccttaaacggcagtgtccgagagatgacaacaaatgttctaaacattttttcacttgcagagg |
| | agggattacgtcgtgatggctctaaacgcgatgcaacagttgaagaaattaaccgctattggaa |
| | atacgcccagctgtgggcagagggagactctgatatgcaaaacaagttgaagattaccaaacg |
| | cttagtcgatgagtaccgccagttttacagagccagtgcgaacgaatccagccatacggttttgc |
| | tgccattttctaaggcactggaacacattctttcggtaccaaatgattgggacgatgaggaaatta |
| | ttgttcaaggggcaggacagttacaggccgcgttagatcggcaggaagtctacaaacgtcccc |
| | tcatgcttgacaaagagattccctttgaaattcgtcggatgaaggagtttgaggcaattcaaacct |
| | tcatgaccacctgcgtcattgacctatttggacaaatgtgtaaaggcgatcgcgcgctcctgcag |
| | gaacatcgcaaccgccttaaatcaggggtcgaatttgcctatcgtcagttagccctgcaagaga |
| | aacaagccaaagccaaccaaaaatctggagaacaatcatgagcattcttgaaaccttaaagcc |
| | agagttccacacagccttccctcgcctcgcctctggcaaatatgtccatttcttcatggtgcgtca |
| | cagccagtcgttccccgtctttcaaaccgatggcgttttgaacactgcccgtactcaggctgggtt |
| | aaatgcaggcgatgaaaaatccagacaggcaatcagtcgcctggtcatgttcaaacggaaaca |
| | ggtgacaccagaacgactcacaggacgggaactgctgcgatcgctgggcattaacggagtag |
| | ttgaaggggaagccaatttctgtgaatacaacggcgaaagatcctgtaaaaaatgccctgattg |
| | cattctctatggttttgcgattggggacagtggctcggaacgttccaaagtctactctgattcagc |
| | cttttccctgagtccttatgaagtttcccatcgcagttttaccttcaatgctccctttgaaggtggaac |
| | catgagtgagaagggtgaaatgcggagtgcgatcaacgaactggatcatgttttgccggaagtt |
| | gcttttccttgcgttgaaacactacgtgaccccacgctggaaggcttcatttatgtaatgggcaac |
| | ctgatccggactcggcgctatggagcgcaggagtcgcgtacagggacaatggcgaatcacat |
| | tattggcgttaccctatgtgatggcgagatcttcagcaatttgcactttacccaagccctctacgat |
| | gccctgaagggtgatttgagtgcgccaattagtgatttatgcagagaggcggcgatcgtggca |
| | gaaaacttgctaaatcaggaacctgtgcggaagatccaagttatttttggagagcagttgaacga |
| | actcctggctgaggtagcgggaatctgccaggatgaaactcgcttgcgaaacactgtgacagc |
| | cttgtaccagcaatcacaagcctatgccgagacctatggtgctgctgctaagaaaggcaaagg |
| | caaatcttaaggtggctgccaatggttatttatcgatgcaatctcacgttgcacgacaacgtattct |
| | tcgccactcgcgagatggggattctctacgaaacagagaagtatctacacaactgggcactca |
| | gctttgctctgtttgaagccacccacatccccaaaccctatcgcttgcagggtgcgatcgctcaa |
| | cgacccggttatctggaagctgacaacgagcaaaaccttttagttctgaatcacgcaggcatcta |
| | tgttttccctgctctaccacttcagtggtcctaccaagtcaataccttcaaagcggctcaagtgcct |
| | tactatggcaaatccaaacagttcggggataagggagctgatcgcaactacccgattaactacg |
| | gacgggcaaaagaactggcagttggcagcacctaccgaacgtatattcttgcccctgaaccaa |
| | tccagatcccccactggatacggttgggtaagtgggcagccaagattcaggtggacgcaaga |
| | cccatacctaactgtggaatccgacaacggtcaggagaatacacctgcgaccatccactaaatc |
| | cgatcgacctgtccggttcaacccgattgctgttatacaaccgcattgtcatgccacccgctagtc |
| | tcgtcagtcagtcgcgagtttctggggactactgggaaatttctaaaccatccaattggactgaat |
| | ttccggactggtcagacctgaaagacatacccaatcaagtttgcttacccagcggagtagcctat |
| | ggagccagtactgccattgccgcatcttaagtccaccgctctgcatatcctggtaattcatttagg |
| | cgttgccgaaagcggagccttgcctgttgaatccggtcgagccgtccatgctcaagttttggact |
| | ggttccgcatggcggatccggcaatttctgaggcaattcacaaaagccaggaatccccgctca |
| | gtctctcaggcctggtcgggaaacgccgtaaagtaggggtgcgggcaggagatgaattttact |
| | tccgaattgggttgttagatgggagtttgattgaaccactgctgagtggtttggagaagtggggc |
| | gacaagcccttttctctggcaaagtgtccctttgttttccgcagtatcaatatgttgccaggaactg |
| | acccctgggttggttcttccgattatgccttgcttacccaaaccccggctactttggataacctagc |
| | tctcaagctcctgtcgccaacaagcttcaagtcgaatgccgggcaaggaatccaaccctttcctt |
| | tgccagagtcagttttcggaaatttacatcgtcgctggaatgtctttgcaccaaaggaattgcagtt |
| | tccaaaaattcaatggagtgggctggtctctgattatgacctcaaaactcaaaagttaaggatgg |
| | aaaactcggttgagatcggcgcagtgggctgggtgcgctatcggtttccagatccagaacaag |
| | cgagaattgcaaccattctggctcactttgccttttttgctggagttgggcgtaagacagcaatgg |
| | ggatgggacaggtggtgttggacaaaaaaacgtagcctaggctgctgccaccgctgagcaat |
| | aactagcataaccccttggggcctctaaacgggtcttgaggggttttttgctgaaacctcaggca |
| | tttgagaagcacacggtcacactgcttccggtagtcaataaaccggtaaaccagcaatagacat |
| | aagcggctatttaacgaccctcgggatctcgacgctctcccttatgcgactcctgcattaggaaa |
| | ttaatacgactcactataggggaattgtgagcggataacaattcccctgtagaaataattttgttta |
| | actttaataaggagatataccatgcttgatgaaaaaagcttcgacaactggtgccaatccgttggt |
| | ctgagccaggcagcacgtgaagttgtagagcgtgtgcgatcctcttcaccctcccgtcgcgtac |
| | agagcggtagaggaaacgtctccggcaattaccctagccgcaaaatgggcgtcacaattcagt |
| | ttgagtcccatcgaaatgagcttgcccgcatctacgaactggagcatgaccccggcgttctgga |
| | atattacgatcaacctcccaaaatcgaacttgattatttgtcaaaaaatgggaggcgtgttcgtact |
| | caatacacacctgatttctttgttatccgtaatgactcagcaggttgggaggagtgtaaaacagag |
| | gatgaccttaagcaattagctgaggacagccctaaccgttaccaacgagctgaggacaatcgc |
| | tggcgatgtccacctgcagaagcctatgctcttccccttgagttgtattttcaggtacggtctagtg |
| | ctgaaattaactggattttccagcaaaactttgtatggctagaagactaccttggtactgaatcactt |
| | actatggatgagtcggttgcagatgcaatcatagccacagtcagagcaaacgagggtatcaca |
| | ctactcgaactgattcaacaattacaaggcacagatgctgatgctatctacatcctgattgcaacc |
| | gaacaaatttatgttgatttgatggctgaacggttagcgaacagcgatcgggcacgggtattttc |
| | cagtccagaaattgctcaagcctttcaactcattacgctggtcgaagcacctctggctaacagttc |
| | tctccatatagttcaggtgggcgttggcacaaccatacactgggatggtgaagtctgggagatt |
| | gttaatacaggctctaccatgacaggattgttgcgatcggacggtaagtttatcgagttgcccaat |
| | tctgcaattgatgaattgattcagaagggcaagattcaatcaatttcagcacaacaagcccctgat |
| | agcaaagcagaagttgaagaaatgcttcgctgtgctcgtccagccgacattaaagaagccaac |
| | cgtcgctacgaaatcattcagccttacctggaaaaggatgcaccagcttgtccaactcgaacaat |
| | tcggcgctggcgtaatcagtattggcaggcagaaaagctttacgggaatggctatgttggtttac |
| | taccaaatcatcagaagaaagggaattatttagttaaggttaatgaagtaactcaagcttttatgctt |
| | gagtttattgagaagcattacgaaaatctcaaacagcgtagtaagcttagagtttatcaggcttttg |
| | tggaggcatgtgaaagccacgaaccgaaactaaaccctccaagccgtactacattcacactag |
| | cgattcagaaacgagcaggacatcagcaaaccttgaaacgtgaaggacgaagagctgccatt |
| | cagaaggaggagttttactgggaactggagctaactactcctcgacacggcgatcgccccttc |
| | gagatcgttcatattgatcacactcaactcgatattgagcttatcagttctttgaactccctggccca |
| | atgcaaccctacatctgttgatgccacttacaatttgggtcgtccttgggcaacgttcatggttgat |
| | gcttacagccgccgtttgctagcagtttacataacctatgaagaacccagctatcgttcctgcatg |
| | atggtgctccgtatttgcgtacagcgatttaagcgttttcctcagacgatcgtggtagataatggtg |
| | cagaatttcacagccattactttgaacaactactggcaacatatcaatgcacaaagaagcacag |
| | gccacctgcaaaagctcgctttggtgcagttgttgaacgattatttggcactgcaaataagcaatt |
| | aattcatgagttgcagggaaatacccaaattactcgcaagcatcgccaagttactcaatctgttca |
| | tccccaaaaattggctctttggacgttaggcgatctctacgaatcactatgtgaatgggcttacaa |
| | agtttatgctgaccgccagcaccctgcgctagggcaaagccctaatgctgcattcgctcaaggg |
| | ctggcgttgggaggtagtcgcatccatcgccgagtagagtatgaccaagtcttccgtatcttgac |
| | attgcccgcaccggaaagaggacaacgcaaagttcagccaggacaagggataaaacttagc |
| | aatatctactactggtcaaatgcctttcgagacccagaaattgaacaatcgatggttgaagttcgtt |
| | atgatccctttgatgccagcattgcctatgcactggtgcgaaaacaatgggtccggtgtatttcct |
| | cctactatcaacagtttcaaaatcgttcagaaaaggaaattcgtttaataactgctgagttgattaa |
| | gcaaaagcgtgaccagggaagagatacaaatgttacggataaagaattggcgcagtttttaag |
| | ctctacagaaatgaaagaaggcaaattcttggagcaacgcttgcggactacagaaaacagggt |
| | agttattcagaatagtacagctacagaagacgatccccccatcactaattctcagaattcttacga |
| | gccatcgcttatgctgccaagtgaggatgagttggatgatcaggaagactggttaaataccggc |
| | aaagctgaggcagaattagaattctatggagagtactagcgatgcaacctcaaataagcttccc |
| | aaaagagttactgcttcaaccaattcctgatcgaattgcctatttcaaagattacacaatggcacat |
| | ccgaagcttatagaagcagaccggaagttaatgcatgctattcgtgaacctggaggagctgctc |
| | ttatctttgtctttggtcctaccggagttggaaagtctactctattgaaacgaattactcaaaagctg |
| | cttaaagaagcattgtctttgatggagactgacaaaggctatattcctattgcgggggtagagata |
| | aaaactccagaggatagtaactttgattggaacgatttttatgttcgtgctcttatggcacttaatga |
| | gccaatgatcgagaaaaaaatcaaccgtacaggtgcaaaactaaaattacggttggcacttgaa |
| | tctgcccttcgtaatcgccgtcctgatgctttttacatagacgaagctcaaaattttggtaaggtag |
| | ccagtgggcgaaaattgcaagatcaaacagattgcattaaatctcttgcaaatctcgctcaaact |
| | cggtttatcttatgtgggatttatgaacttttaccactacgaaatctaagtgggcagctatgccgcc |
| | gtagccatagtattcactttccacgttattcggcagattctgagcaggatctaaaggcctttaaaag |
| | tgcgttattaacactacaaaatcaactccctcttcccgaaccacctgatttagtggcgcattggga |
| | attttgttatgagagaactatcggctgtattggtattttgaaagatttgctccttcggactctaagcg |
| | cgattttagtacgggatggagacaaagttttgacattgaagcccaaggatttagaacaacgtgca |
| | tggtctttagaagaatgtttggttatgctgagagaggcaaaggaaggagagcaacaacttactg |
| | acaaatcgggcttacatgaggaattacggacagctttagggtttaatgcagctacgtccaagca |
| | gtctgccaaatcatccaagtcaacatcaagcaaaaaagcaggcagtgttggaaagccttcacct |
| | cgtcgtcgttcagttggaatagatcaagatgccagctaacgatctagaaatctatgaatcttggaa |
| | tttagatctccctttactacctaatcgaagtaggctctaccacttgaaaccgattgcaattggagat |
| | gcttacgttgaaggattaatcagctatgtttgccgtttggctgaagcccactgtgtttctccaggag |
| | tccttactaaacaggaaatattgcccagtctaagaaaagtttataatgtgggttcaagagaagttc |
| | atgaagtacgaagagatgaaagtttagtttctatttcctcctttcctaagccagaaagcaataaaaa |
| | tcctaatgaaccaggcttctgggcatggcaatatattgaaggattgcagcctttaactcttagaga |
| | aaaccttgaagcattaactattccaaattggaaaaagcaaattccacaggtgacaggaattgag |
| | gttgcgcgtgagttgagagcttggtgccctgaatgctttcaacaatggcgcaagactaatcagat |
| | aatttatgaaccgatgctttggtcgatcgctttagttaccgtatgtccttatcattaccttccgctaca |
| | aactcgctgtccttattgtaaaaaaactcaacggcctatcacaggaaaaatgcaggtagctcgct |
| | gttccgaatgtttagagtggcttggcattcaaccaagcgaagattcagagttagaaatcaaaatta |
| | aatctgaagtcgaatggcaccattgggttgcggagaaagtaatggagattttggtatttaaagatt |
| | atactttaactgcaaatgctataacaatagcagctccaattacgaaagaaaaatattttcaactaga |
| | atcgagcttaataaattgtccaattaagtatgctcaacagcagttcttaaagctcttcttcaattaac |
| | acaaaaacgcccagttttaacttaagtcgaacagaaagtaatcgtattgtacacggccgcataat |
| | cgaaattaatacgactcactataggggaattgtgagcggataacaattccccatcttagtatatta |
| | gttaagtataagaaggagatatacatatggcagatctcaattggatatcggccggccacgcgat |
| | cgctgacgtcggtaccctcgagtctggtaaagaaaccgctgctgcgaaatttgaacgccagca |
| | catggactcgtctactagcgcagcttaattaacctaggctgctgccaccgctgagcaataactag |
| | cataaccccttggggcctctaaacgggtcttgaggggttttttgctgaaacctcaggcatttgaga |
| | agcacacggtcacactgcttccggtagtgccctgaaccgacgaccgggtcatcgtggccggat |
| | cttgcggcccctcggcttgaacgaattgttagacattatttgccgactaccttggtgatctcgcctt |
| | tcacgtagtggacaaattcttccaactgatctgcgcgcgaggccaagcgatcttcttcttgtccaa |
| | gataagcctgtctagcttcaagtatgacgggctgatactgggccggcaggcgctccattgccca |
| | gtcggcagcgacatccttcggcgcgattttgccggttactgcgctgtaccaaatgcgggacaac |
| | gtaagcactacatttcgctcatcgccagcccagtcgggcggcgagttccatagcgttaaggtttc |
| | atttagcgcctcaaatagatcctgttcaggaaccggatcaaagagttcctccgccgctggaccta |
| | ccaaggcaacgctatgttctcttgcttttgtcagcaagatagccagatcaatgtcgatcgtggctg |
| | gctcgaagatacctgcaagaatgtcattgcgctgccattctccaaattgcagttcgcgcttagctg |
| | gataacgccacggaatgatgtcgtcgtgcacaacaatggtgacttctacagcgcggagaatctc |
| | gctctctccaggggaagccgaagtttccaaaaggtcgttgatcaaagctcgccgcgttgtttcat |
| | caagccttacggtcaccgtaaccagcaaatcaatatcactgtgtggcttcaggccgccatccact |
| | gcggagccgtacaaatgtacggccagcaacgtcggttcgagatggcgctcgatgacgccaac |
| | tacctctgatagttgagtcgatacttcggcgatcaccgcttccctcatactcttcctttttcaatatta |
| | ttgaagcatttatcagggttattgtctcatgagcggatacatatttgaatgtatttagaaaaataaaca |
| | aatagctagctcactcggtcgctacgctccgggcgtgagactgcggcgggcgctgcggacac |
| | atacaaagttacccacagattccgtggataagcaggggactaacatgtgaggcaaaacagcag |
| | ggccgcgccggtggcgtttttccataggctccgccctcctgccagagttcacataaacagacgc |
| | ttttccggtgcatctgtgggagccgtgaggctcaaccatgaatctgacagtacgggcgaaaccc |
| | gacaggacttaaagatccccaccgtttccggcgggtcgctccctcttgcgctctcctgttccgac |
| | cctgccgtttaccggatacctgttccgcctttctcccttacgggaagtgtggcgctttctcatagct |
| | cacacactggtatctcggctcggtgtaggtcgttcgctccaagctgggctgtaagcaagaactc |
| | cccgttcagcccgactgctgcgccttatccggtaactgttcacttgagtccaacccggaaaagc |
| | acggtaaaacgccactggcagcagccattggtaactgggagttcgcagaggatttgtttagcta |
| | aacacgcggttgctcttgaagtgtgcgccaaagtccggctacactggaaggacagatttggttg |
| | ctgtgctctgcgaaagccagttaccacggttaagcagttccccaactgacttaaccttcgatcaa |
| | accacctccccaggtggttttttcgtttacagggcaaaagattacgcgcagaaaaaaaggatctc |
| | aagaagatcctttgatcttttctactgaaccgctctagatttcagtgcaatttatctcttcaaatgtag |
| | cacctgaagtcagccccatacgatataagttgtaattctcatgttagtcatgccccgcgcccacc |
| | ggaaggagctgactgggttgaaggctctcaagggcatcggtcgagatcccggtgcctaatga |
| | gtgagctaacttacattaattgcgttgcgctcactgcccgctttccagtcgggaaacctgtcgtgc |
| | cagctgcattaatgaatcggccaacgcgcggggagaggcggtttgcgtattgggcgccaggg |
| | tggtttttcttttcaccagtgagacgggcaacagctgattgcccttcaccgcctggccctgagag |
| | agttgcagcaagcggtccacgctggtttgccccagcaggcgaaaatcctgtttgatggtggtta |
| | acggcgggatataacatgagctgtcttcggtatcgtcgtatcccactaccgagatgtccgcacc |
| | aacgcgcagcccggactcggtaatggcgcgcattgcgcccagcgccatctgatcgttggcaa |
| | ccagcatcgcagtgggaacgatgccctcattcagcatttgcatggtttgttgaaaaccggacatg |
| | gcactccagtcgccttcccgttccgctatcggctgaatttgattgcgagtgagatatttatgccag |
| | ccagccagacgcagacgcgccgagacagaacttaatgggcccgctaacagcgcgatttgctg |
| | gtgacccaatgcgaccagatgctccacgcccagtcgcgtaccgtcttcatgggagaaaataat |
| | actgttgatgggtgtctggtcagagacatcaagaaataacgccggaacattagtgcaggcagct |
| | tccacagcaatggcatcctggtcatccagcggatagttaatgatcagcccactgacgcgttgcg |
| | cgagaagattgtgcaccgccgctttacaggcttcgacgccgcttcgttctaccatcgacaccac |
| | cacgctggcacccagttgatcggcgcgagatttaatcgccgcgacaatttgcgacggcgcgtg |
| | cagggccagactggaggtggcaacgccaatcagcaacgactgtttgcccgccagttgttgtgc |
| | cacgcggttgggaatgtaattcagctccgccatcgccgcttccactttttcccgcgttttcgcaga |
| | aacgtggctggcctggttcaccacgcgggaaacggtctgataagagacaccggcatactctgc |
| | gacatcgtataacgttactggtttcacattcaccaccctgaattgactctcttccgggcgctatcat |
| | gccataccgcgaaaggttttgcgccattcgatggtgtccgggatctcgacgctctcccttatgcg |
| | actcctgcattaggaaattaatacgactcactata (SEQ ID NO: 94) |
| pHelper(CyCAST)_ | ggggaattgtgagcggataacaattcccctgtagaaataattttgtttaactttaataaggagatat |
| Shuffled-spacer_ | accatggtttccaagcctgaatcccggtctacgggactgaaacaccatctatgtatagctacgat |
| ΔTnsD | ggaatagtaaagggtttccaagcctgaatcccggtctacgggactgaaacgatccgaattcga |
| | gctcggcgcgcctgcaggtcgacaagcttgcggccgcataatgcttaagtcgaacagaaagta |
| | atcgtattgtacacggccgcataatcgaaattaatacgactcactataggggaattgtgagcgga |
| | taacaattccccatcttagtatattagttaagtataagaaggagatatacatatggctaccctactcc |
| | aaaccctcctgttacgaacgttgtcacccgaggcggatccagttctctatgtctacattgagacg |
| | gtactacctgcaatggaacgggaatttggcttgattccagcccttggtggctcggaggaagcac |
| | actttcatctgctgaggcgacggggaaacaaatatgcggcggaaaattccaagcggtacgcca |
| | gtcgctctgatcaaagcctgctggttcatgtgctcaatgccttgctgaccgcctggaatttgttgc |
| | cattcctaagctccacatttgaactatcagatgatgagaaacggctgctctgcctggggctgacc |
| | ctacacgattacaacaagtggtgccagggtgaggaggaagattcgccgattgcatgcgaagta |
| | gcagagattcttgagctttgtcaaacattgggagagcggttgaactttgacggattctgggcaga |
| | gtggcgggagtatttgacagagattgcctaccttgcccaaaacacccagggaaaagttggaac |
| | aaatctacccaaagcgaattggggcagcttcaaaattctagatacccgtcgtctggattcgcccc |
| | tacgatggctactcgcttttggcgatgtcgcagtccatctcagcgatccatctgacatcgcaatga |
| | aaacggggggagatcgcctgcgggaacatctgcgctttctaggtattcgtcgcaagctggtcta |
| | ccatcgcctgcgcaactgtacagggctattaaccaacggaattcataatgcggtaatgcattttg |
| | ctagagaactggattggcaacccatcctcttctttgctcagggtgttgtttatctcgctcctcaggg |
| | tgctggcgttccggatcgcttagaacttcaggaatttctctgggagcaggtcagcaaccgattag |
| | ccaccagcatgttgcggggagaggttggtttcaaacgggatggcaagggactcaaggtggct |
| | ccccaaaccctggaactgttctcccctgctcagttgattcggaacttgcccggtgttgtccaatctt |
| | atgtcggaaacgctaaatctcctgcgactccaaaacgacttgagaagctggaactcagttcaac |
| | agagcgagagtttttggctaaaggtgcagatttaagagcagatcgcattgccgagtttatctttct |
| | ggcgcaaaaagagtttttcggagataaaccagaatttatcacttggatgcttgaaactctggggg |
| | ttcaaactgctatcacttcggagcagacgcagcttcaatccggtggtgtgaactggggatggtat |
| | cacgcagcagcctattacgtcgcgagcaatgccacgctggatttggaagagatttcggagaaa |
| | cttcaggaatttgccgagcagttggcaacctgggcggaggaaaacaatttcttcccagagcatc |
| | gcagcccaacccatgacatctttttcaactatctgactcaatacctggaagttcaggggtgggag |
| | cagcaggagcagtcctttcagcttgaactggcaggctatgttggcgcaaagactaaaactgcca |
| | gacaaccgatttgctccctcagttccggtgaatttgcttcggaagaccagatggattctgttgtcct |
| | gttcaaaccgcagcagtacagcaacaagaatcctctcggtggacggcagatcaagcggggg |
| | atttccaaaatctggtccctggaaatgctgctcaggcaggctctttggtctgcgccagcaggcaa |
| | actagaggaacaacaacccattttcctttacatcttcccggcatacgtttactcgccccaggttgct |
| | gctgctgtccggttactggtgaatgatctgaagcgtgtcaacctctgggaagtccgtaaacactg |
| | gttggagaatggcatggatgctcaggcgttgcgatcgtttccctggctagtcggtgacaaaaac |
| | gattcgggtaactctgctccatccagatactctggtgaagacttgcctttcatggcaatgaactac |
| | accaccaccagaggcaaaacgataactgatgcctgggttgaacctgctttcctgacactggtgc |
| | tgcctttgttgctgggtgtgaaagttgtcgccaccagtagccagattccactctatgaaagcgac |
| | aacgacttttgcgaatctgtcaagctggatggacccgccgatttctgggctttgctgcggctacc |
| | aacttccctgcatctccaggattggattggggggcgtgttcaaaatttagcagaaggcttgaacc |
| | gcttgatggttgcctactgcctgcatctggacagtcgtagcgctccaccagatgcgcgctggag |
| | agccttaaacggcagtgtccgagagatgacaacaaatgttctaaacattttttcacttgcagagg |
| | agggattacgtcgtgatggctctaaacgcgatgcaacagttgaagaaattaaccgctattggaa |
| | atacgcccagctgtgggcagagggagactctgatatgcaaaacaagttgaagattaccaaacg |
| | cttagtcgatgagtaccgccagttttacagagccagtgcgaacgaatccagccatacggttttgc |
| | tgccattttctaaggcactggaacacattctttcggtaccaaatgattgggacgatgaggaaatta |
| | ttgttcaaggggcaggacagttacaggccgcgttagatcggcaggaagtctacaaacgtcccc |
| | tcatgcttgacaaagagattccctttgaaattcgtcggatgaaggagtttgaggcaattcaaacct |
| | tcatgaccacctgcgtcattgacctatttggacaaatgtgtaaaggcgatcgcgcgctcctgcag |
| | gaacatcgcaaccgccttaaatcaggggtcgaatttgcctatcgtcagttagccctgcaagaga |
| | aacaagccaaagccaaccaaaaatctggagaacaatcatgagcattcttgaaaccttaaagcc |
| | agagttccacacagccttccctcgcctcgcctctggcaaatatgtccatttcttcatggtgcgtca |
| | cagccagtcgttccccgtctttcaaaccgatggcgttttgaacactgcccgtactcaggctgggtt |
| | aaatgcaggcgatgaaaaatccagacaggcaatcagtcgcctggtcatgttcaaacggaaaca |
| | ggtgacaccagaacgactcacaggacgggaactgctgcgatcgctgggcattaacggagtag |
| | ttgaaggggaagccaatttctgtgaatacaacggcgaaagatcctgtaaaaaatgccctgattg |
| | cattctctatggttttgcgattggggacagtggctcggaacgttccaaagtctactctgattcagc |
| | cttttccctgagtccttatgaagtttcccatcgcagttttaccttcaatgctccctttgaaggtggaac |
| | catgagtgagaagggtgaaatgcggagtgcgatcaacgaactggatcatgttttgccggaagtt |
| | gcttttccttgcgttgaaacactacgtgaccccacgctggaaggcttcatttatgtaatgggcaac |
| | ctgatccggactcggcgctatggagcgcaggagtcgcgtacagggacaatggcgaatcacat |
| | tattggcgttaccctatgtgatggcgagatcttcagcaatttgcactttacccaagccctctacgat |
| | gccctgaagggtgatttgagtgcgccaattagtgatttatgcagagaggcggcgatcgtggca |
| | gaaaacttgctaaatcaggaacctgtgcggaagatccaagttatttttggagagcagttgaacga |
| | actcctggctgaggtagcgggaatctgccaggatgaaactcgcttgcgaaacactgtgacagc |
| | cttgtaccagcaatcacaagcctatgccgagacctatggtgctgctgctaagaaaggcaaagg |
| | caaatcttaaggtggctgccaatggttatttatcgatgcaatctcacgttgcacgacaacgtattct |
| | tcgccactcgcgagatggggattctctacgaaacagagaagtatctacacaactgggcactca |
| | gctttgctctgtttgaagccacccacatccccaaaccctatcgcttgcagggtgcgatcgctcaa |
| | cgacccggttatctggaagctgacaacgagcaaaaccttttagttctgaatcacgcaggcatcta |
| | tgttttccctgctctaccacttcagtggtcctaccaagtcaataccttcaaagcggctcaagtgcct |
| | tactatggcaaatccaaacagttcggggataagggagctgatcgcaactacccgattaactacg |
| | gacgggcaaaagaactggcagttggcagcacctaccgaacgtatattcttgcccctgaaccaa |
| | tccagatcccccactggatacggttgggtaagtgggcagccaagattcaggtggacgcaaga |
| | cccatacctaactgtggaatccgacaacggtcaggagaatacacctgcgaccatccactaaatc |
| | cgatcgacctgtccggttcaacccgattgctgttatacaaccgcattgtcatgccacccgctagtc |
| | tcgtcagtcagtcgcgagtttctggggactactgggaaatttctaaaccatccaattggactgaat |
| | ttccggactggtcagacctgaaagacatacccaatcaagtttgcttacccagcggagtagcctat |
| | ggagccagtactgccattgccgcatcttaagtccaccgctctgcatatcctggtaattcatttagg |
| | cgttgccgaaagcggagccttgcctgttgaatccggtcgagccgtccatgctcaagttttggact |
| | ggttccgcatggcggatccggcaatttctgaggcaattcacaaaagccaggaatccccgctca |
| | gtctctcaggcctggtcgggaaacgccgtaaagtaggggtgcgggcaggagatgaattttact |
| | tccgaattgggttgttagatgggagtttgattgaaccactgctgagtggtttggagaagtggggc |
| | gacaagcccttttctctggcaaagtgtccctttgttttccgcagtatcaatatgttgccaggaactg |
| | acccctgggttggttcttccgattatgccttgcttacccaaaccccggctactttggataacctagc |
| | tctcaagctcctgtcgccaacaagcttcaagtcgaatgccgggcaaggaatccaaccctttcctt |
| | tgccagagtcagttttcggaaatttacatcgtcgctggaatgtctttgcaccaaaggaattgcagtt |
| | tccaaaaattcaatggagtgggctggtctctgattatgacctcaaaactcaaaagttaaggatgg |
| | aaaactcggttgagatcggcgcagtgggctgggtgcgctatcggtttccagatccagaacaag |
| | cgagaattgcaaccattctggctcactttgccttttttgctggagttgggcgtaagacagcaatgg |
| | ggatgggacaggtggtgttggacaaaaaaacgtagcctaggctgctgccaccgctgagcaat |
| | aactagcataaccccttggggcctctaaacgggtcttgaggggttttttgctgaaacctcaggca |
| | tttgagaagcacacggtcacactgcttccggtagtcaataaaccggtaaaccagcaatagacat |
| | aagcggctatttaacgaccctcgggatctcgacgctctcccttatgcgactcctgcattaggaaa |
| | ttaatacgactcactataggggaattgtgagcggataacaattcccctgtagaaataattttgttta |
| | actttaataaggagatataccatgcttgatgaaaaaagcttcgacaactggtgccaatccgttggt |
| | ctgagccaggcagcacgtgaagttgtagagcgtgtgcgatcctcttcaccctcccgtcgcgtac |
| | agagcggtagaggaaacgtctccggcaattaccctagccgcaaaatgggcgtcacaattcagt |
| | ttgagtcccatcgaaatgagcttgcccgcatctacgaactggagcatgaccccggcgttctgga |
| | atattacgatcaacctcccaaaatcgaacttgattatttgtcaaaaaatgggaggcgtgttcgtact |
| | caatacacacctgatttctttgttatccgtaatgactcagcaggttgggaggagtgtaaaacagag |
| | gatgaccttaagcaattagctgaggacagccctaaccgttaccaacgagctgaggacaatcgc |
| | tggcgatgtccacctgcagaagcctatgctcttccccttgagttgtattttcaggtacggtctagtg |
| | ctgaaattaactggattttccagcaaaactttgtatggctagaagactaccttggtactgaatcactt |
| | actatggatgagtcggttgcagatgcaatcatagccacagtcagagcaaacgagggtatcaca |
| | ctactcgaactgattcaacaattacaaggcacagatgctgatgctatctacatcctgattgcaacc |
| | gaacaaatttatgttgatttgatggctgaacggttagcgaacagcgatcgggcacgggtattttc |
| | cagtccagaaattgctcaagcctttcaactcattacgctggtcgaagcacctctggctaacagttc |
| | tctccatatagttcaggtgggcgttggcacaaccatacactgggatggtgaagtctgggagatt |
| | gttaatacaggctctaccatgacaggattgttgcgatcggacggtaagtttatcgagttgcccaat |
| | tctgcaattgatgaattgattcagaagggcaagattcaatcaatttcagcacaacaagcccctgat |
| | agcaaagcagaagttgaagaaatgcttcgctgtgctcgtccagccgacattaaagaagccaac |
| | cgtcgctacgaaatcattcagccttacctggaaaaggatgcaccagcttgtccaactcgaacaat |
| | tcggcgctggcgtaatcagtattggcaggcagaaaagctttacgggaatggctatgttggtttac |
| | taccaaatcatcagaagaaagggaattatttagttaaggttaatgaagtaactcaagcttttatgctt |
| | gagtttattgagaagcattacgaaaatctcaaacagcgtagtaagcttagagtttatcaggcttttg |
| | tggaggcatgtgaaagccacgaaccgaaactaaaccctccaagccgtactacattcacactag |
| | cgattcagaaacgagcaggacatcagcaaaccttgaaacgtgaaggacgaagagctgccatt |
| | cagaaggaggagttttactgggaactggagctaactactcctcgacacggcgatcgccccttc |
| | gagatcgttcatattgatcacactcaactcgatattgagcttatcagttctttgaactccctggccca |
| | atgcaaccctacatctgttgatgccacttacaatttgggtcgtccttgggcaacgttcatggttgat |
| | gcttacagccgccgtttgctagcagtttacataacctatgaagaacccagctatcgttcctgcatg |
| | atggtgctccgtatttgcgtacagcgatttaagcgttttcctcagacgatcgtggtagataatggtg |
| | cagaatttcacagccattactttgaacaactactggcaacatatcaatgcacaaagaagcacag |
| | gccacctgcaaaagctcgctttggtgcagttgttgaacgattatttggcactgcaaataagcaatt |
| | aattcatgagttgcagggaaatacccaaattactcgcaagcatcgccaagttactcaatctgttca |
| | tccccaaaaattggctctttggacgttaggcgatctctacgaatcactatgtgaatgggcttacaa |
| | agtttatgctgaccgccagcaccctgcgctagggcaaagccctaatgctgcattcgctcaaggg |
| | ctggcgttgggaggtagtcgcatccatcgccgagtagagtatgaccaagtcttccgtatcttgac |
| | attgcccgcaccggaaagaggacaacgcaaagttcagccaggacaagggataaaacttagc |
| | aatatctactactggtcaaatgcctttcgagacccagaaattgaacaatcgatggttgaagttcgtt |
| | atgatccctttgatgccagcattgcctatgcactggtgcgaaaacaatgggtccggtgtatttcct |
| | cctactatcaacagtttcaaaatcgttcagaaaaggaaattcgtttaataactgctgagttgattaa |
| | gcaaaagcgtgaccagggaagagatacaaatgttacggataaagaattggcgcagtttttaag |
| | ctctacagaaatgaaagaaggcaaattcttggagcaacgcttgcggactacagaaaacagggt |
| | agttattcagaatagtacagctacagaagacgatccccccatcactaattctcagaattcttacga |
| | gccatcgcttatgctgccaagtgaggatgagttggatgatcaggaagactggttaaataccggc |
| | aaagctgaggcagaattagaattctatggagagtactagcgatgcaacctcaaataagcttccc |
| | aaaagagttactgcttcaaccaattcctgatcgaattgcctatttcaaagattacacaatggcacat |
| | ccgaagcttatagaagcagaccggaagttaatgcatgctattcgtgaacctggaggagctgctc |
| | ttatctttgtctttggtcctaccggagttggaaagtctactctattgaaacgaattactcaaaagctg |
| | cttaaagaagcattgtctttgatggagactgacaaaggctatattcctattgcgggggtagagata |
| | aaaactccagaggatagtaactttgattggaacgatttttatgttcgtgctcttatggcacttaatga |
| | gccaatgatcgagaaaaaaatcaaccgtacaggtgcaaaactaaaattacggttggcacttgaa |
| | tctgcccttcgtaatcgccgtcctgatgctttttacatagacgaagctcaaaattttggtaaggtag |
| | ccagtgggcgaaaattgcaagatcaaacagattgcattaaatctcttgcaaatctcgctcaaact |
| | cggtttatcttatgtgggatttatgaacttttaccactacgaaatctaagtgggcagctatgccgcc |
| | gtagccatagtattcactttccacgttattcggcagattctgagcaggatctaaaggcctttaaaag |
| | tgcgttattaacactacaaaatcaactccctcttcccgaaccacctgatttagtggcgcattggga |
| | attttgttatgagagaactatcggctgtattggtattttgaaagatttgctccttcggactctaagcg |
| | cgattttagtacgggatggagacaaagttttgacattgaagcccaaggatttagaacaacgtgca |
| | tggtctttagaagaatgtttggttatgctgagagaggcaaaggaaggagagcaacaacttactg |
| | acaaatcgggcttacatgaggaattacggacagctttagggtttaatgcagctacgtccaagca |
| | gtctgccaaatcatccaagtcaacatcaagcaaaaaagcaggcagtgttggaaagccttcacct |
| | cgtcgtcgttcagttggaatagatcaagatgccagctaacgatctagaaatctatgaatcttggaa |
| | tttagatctccctttactacctaatcgaagtaggctctaccacttgaaaccgattgcaattggagat |
| | gcttacgttgaaggattaatcagctatgtttgccgtttggctgaagcccactgtgtttctccaggag |
| | tccttactaaacaggaaatattgcccagtctaagaaaagtttataatgtgggttcaagagaagttc |
| | atgaagtacgaagagatgaaagtttagtttctatttcctcctttcctaagccagaaagcaataaaaa |
| | tcctaatgaaccaggcttctgggcatggcaatatattgaaggattgcagcctttaactcttagaga |
| | aaaccttgaagcattaactattccaaattggaaaaagcaaattccacaggtgacaggaattgag |
| | gttgcgcgtgagttgagagcttggtgccctgaatgctttcaacaatggcgcaagactaatcagat |
| | aatttatgaaccgatgctttggtcgatcgctttagttaccgtatgtccttatcattaccttccgctaca |
| | aactcgctgtccttattgtaaaaaaactcaacggcctatcacaggaaaaatgcaggtagctcgct |
| | gttccgaatgtttagagtggcttggcattcaaccaagcgaagattcagagttagaaatcaaaatta |
| | aatctgaagtcgaatggcaccattgggttgcggagaaagtaatggagattttggtatttaaagatt |
| | atactttaactgcaaatgctataacaatagcagctccaattacgaaagaaaaatattttcaactaga |
| | atcgagcttaataaattgtccaattaagtatgctcaacagcagttcttaaagctcttcttcaattaac |
| | acaaaaacgcccagttttaacttaagtcgaacagaaagtaatcgtattgtacacggccgcataat |
| | cgaaattaatacgactcactataggggaattgtgagcggataacaattccccatcttagtatatta |
| | gttaagtataagaaggagatatacatatggcagatctcaattggatatcggccggccacgcgat |
| | cgctgacgtcggtaccctcgagtctggtaaagaaaccgctgctgcgaaatttgaacgccagca |
| | catggactcgtctactagcgcagcttaattaacctaggctgctgccaccgctgagcaataactag |
| | cataaccccttggggcctctaaacgggtcttgaggggttttttgctgaaacctcaggcatttgaga |
| | agcacacggtcacactgcttccggtagtgccctgaaccgacgaccgggtcatcgtggccggat |
| | cttgcggcccctcggcttgaacgaattgttagacattatttgccgactaccttggtgatctcgcctt |
| | tcacgtagtggacaaattcttccaactgatctgcgcgcgaggccaagcgatcttcttcttgtccaa |
| | gataagcctgtctagcttcaagtatgacgggctgatactgggccggcaggcgctccattgccca |
| | gtcggcagcgacatccttcggcgcgattttgccggttactgcgctgtaccaaatgcgggacaac |
| | gtaagcactacatttcgctcatcgccagcccagtcgggcggcgagttccatagcgttaaggtttc |
| | atttagcgcctcaaatagatcctgttcaggaaccggatcaaagagttcctccgccgctggaccta |
| | ccaaggcaacgctatgttctcttgcttttgtcagcaagatagccagatcaatgtcgatcgtggctg |
| | gctcgaagatacctgcaagaatgtcattgcgctgccattctccaaattgcagttcgcgcttagctg |
| | gataacgccacggaatgatgtcgtcgtgcacaacaatggtgacttctacagcgcggagaatctc |
| | gctctctccaggggaagccgaagtttccaaaaggtcgttgatcaaagctcgccgcgttgtttcat |
| | caagccttacggtcaccgtaaccagcaaatcaatatcactgtgtggcttcaggccgccatccact |
| | gcggagccgtacaaatgtacggccagcaacgtcggttcgagatggcgctcgatgacgccaac |
| | tacctctgatagttgagtcgatacttcggcgatcaccgcttccctcatactcttcctttttcaatatta |
| | ttgaagcatttatcagggttattgtctcatgagcggatacatatttgaatgtatttagaaaaataaaca |
| | aatagctagctcactcggtcgctacgctccgggcgtgagactgcggcgggcgctgcggacac |
| | atacaaagttacccacagattccgtggataagcaggggactaacatgtgaggcaaaacagcag |
| | ggccgcgccggtggcgtttttccataggctccgccctcctgccagagttcacataaacagacgc |
| | ttttccggtgcatctgtgggagccgtgaggctcaaccatgaatctgacagtacgggcgaaaccc |
| | gacaggacttaaagatccccaccgtttccgggggtcgctccctcttgcgctctcctgttccgac |
| | cctgccgtttaccggatacctgttccgcctttctcccttacgggaagtgtggcgctttctcatagct |
| | cacacactggtatctcggctcggtgtaggtcgttcgctccaagctgggctgtaagcaagaactc |
| | cccgttcagcccgactgctgcgccttatccggtaactgttcacttgagtccaacccggaaaagc |
| | acggtaaaacgccactggcagcagccattggtaactgggagttcgcagaggatttgtttagcta |
| | aacacgcggttgctcttgaagtgtgcgccaaagtccggctacactggaaggacagatttggttg |
| | ctgtgctctgcgaaagccagttaccacggttaagcagttccccaactgacttaaccttcgatcaa |
| | accacctccccaggtggttttttcgtttacagggcaaaagattacgcgcagaaaaaaaggatctc |
| | aagaagatcctttgatcttttctactgaaccgctctagatttcagtgcaatttatctcttcaaatgtag |
| | cacctgaagtcagccccatacgatataagttgtaattctcatgttagtcatgccccgcgcccacc |
| | ggaaggagctgactgggttgaaggctctcaagggcatcggtcgagatcccggtgcctaatga |
| | gtgagctaacttacattaattgcgttgcgctcactgcccgctttccagtcgggaaacctgtcgtgc |
| | cagctgcattaatgaatcggccaacgcgcggggagaggcggtttgcgtattgggcgccaggg |
| | tggtttttcttttcaccagtgagacgggcaacagctgattgcccttcaccgcctggccctgagag |
| | agttgcagcaagcggtccacgctggtttgccccagcaggcgaaaatcctgtttgatggtggtta |
| | acggcgggatataacatgagctgtcttcggtatcgtcgtatcccactaccgagatgtccgcacc |
| | aacgcgcagcccggactcggtaatggcgcgcattgcgcccagcgccatctgatcgttggcaa |
| | ccagcatcgcagtgggaacgatgccctcattcagcatttgcatggtttgttgaaaaccggacatg |
| | gcactccagtcgccttcccgttccgctatcggctgaatttgattgcgagtgagatatttatgccag |
| | ccagccagacgcagacgcgccgagacagaacttaatgggcccgctaacagcgcgatttgctg |
| | gtgacccaatgcgaccagatgctccacgcccagtcgcgtaccgtcttcatgggagaaaataat |
| | actgttgatgggtgtctggtcagagacatcaagaaataacgccggaacattagtgcaggcagct |
| | tccacagcaatggcatcctggtcatccagcggatagttaatgatcagcccactgacgcgttgcg |
| | cgagaagattgtgcaccgccgctttacaggcttcgacgccgcttcgttctaccatcgacaccac |
| | cacgctggcacccagttgatcggcgcgagatttaatcgccgcgacaatttgcgacggcgcgtg |
| | cagggccagactggaggtggcaacgccaatcagcaacgactgtttgcccgccagttgttgtgc |
| | cacgcggttgggaatgtaattcagctccgccatcgccgcttccactttttcccgcgttttcgcaga |
| | aacgtggctggcctggttcaccacgcgggaaacggtctgataagagacaccggcatactctgc |
| | gacatcgtataacgttactggtttcacattcaccaccctgaattgactctcttccgggcgctatcat |
| | gccataccgcgaaaggttttgcgccattcgatggtgtccgggatctcgacgctctcccttatgcg |
| | actcctgcattaggaaattaatacgactcactata (SEQ ID NO: 95) |
| pHelper(CyCAST)_PSP | ggggaattgtgagcggataacaattcccctgtagaaataattttgtttaactttaataaggagatat |
| 1_ΔTniQΔTnsD | accatggtttccaagcctgaatcccggtctacgggactgaaacgagaagtcatttaataaggcc |
| | actgttaaacgtaggtttccaagcctgaatcccggtctacgggactgaaacgatccgaattcga |
| | gctcggcgcgcctgcaggtcgacaagcttgcggccgcataatgcttaagtcgaacagaaagta |
| | atcgtattgtacacggccgcataatcgaaattaatacgactcactataggggaattgtgagcgga |
| | taacaattccccatcttagtatattagttaagtataagaaggagatatacatatggctaccctactcc |
| | aaaccctcctgttacgaacgttgtcacccgaggcggatccagttctctatgtctacattgagacg |
| | gtactacctgcaatggaacgggaatttggcttgattccagcccttggtggctcggaggaagcac |
| | actttcatctgctgaggcgacggggaaacaaatatgcggcggaaaattccaagcggtacgcca |
| | gtcgctctgatcaaagcctgctggttcatgtgctcaatgccttgctgaccgcctggaatttgttgc |
| | cattcctaagctccacatttgaactatcagatgatgagaaacggctgctctgcctggggctgacc |
| | ctacacgattacaacaagtggtgccagggtgaggaggaagattcgccgattgcatgcgaagta |
| | gcagagattcttgagctttgtcaaacattgggagagcggttgaactttgacggattctgggcaga |
| | gtggcgggagtatttgacagagattgcctaccttgcccaaaacacccagggaaaagttggaac |
| | aaatctacccaaagcgaattggggcagcttcaaaattctagatacccgtcgtctggattcgcccc |
| | tacgatggctactcgcttttggcgatgtcgcagtccatctcagcgatccatctgacatcgcaatga |
| | aaacggggggagatcgcctgcgggaacatctgcgctttctaggtattcgtcgcaagctggtcta |
| | ccatcgcctgcgcaactgtacagggctattaaccaacggaattcataatgcggtaatgcattttg |
| | ctagagaactggattggcaacccatcctcttctttgctcagggtgttgtttatctcgctcctcaggg |
| | tgctggcgttccggatcgcttagaacttcaggaatttctctgggagcaggtcagcaaccgattag |
| | ccaccagcatgttgcggggagaggttggtttcaaacgggatggcaagggactcaaggtggct |
| | ccccaaaccctggaactgttctcccctgctcagttgattcggaacttgcccggtgttgtccaatctt |
| | atgtcggaaacgctaaatctcctgcgactccaaaacgacttgagaagctggaactcagttcaac |
| | agagcgagagtttttggctaaaggtgcagatttaagagcagatcgcattgccgagtttatctttct |
| | ggcgcaaaaagagtttttcggagataaaccagaatttatcacttggatgcttgaaactctggggg |
| | ttcaaactgctatcacttcggagcagacgcagcttcaatccggtggtgtgaactggggatggtat |
| | cacgcagcagcctattacgtcgcgagcaatgccacgctggatttggaagagatttcggagaaa |
| | cttcaggaatttgccgagcagttggcaacctgggcggaggaaaacaatttcttcccagagcatc |
| | gcagcccaacccatgacatctttttcaactatctgactcaatacctggaagttcaggggtgggag |
| | cagcaggagcagtcctttcagcttgaactggcaggctatgttggcgcaaagactaaaactgcca |
| | gacaaccgatttgctccctcagttccggtgaatttgcttcggaagaccagatggattctgttgtcct |
| | gttcaaaccgcagcagtacagcaacaagaatcctctcggtggacggcagatcaagcggggg |
| | atttccaaaatctggtccctggaaatgctgctcaggcaggctctttggtctgcgccagcaggcaa |
| | actagaggaacaacaacccattttcctttacatcttcccggcatacgtttactcgccccaggttgct |
| | gctgctgtccggttactggtgaatgatctgaagcgtgtcaacctctgggaagtccgtaaacactg |
| | gttggagaatggcatggatgctcaggcgttgcgatcgtttccctggctagtcggtgacaaaaac |
| | gattcgggtaactctgctccatccagatactctggtgaagacttgcctttcatggcaatgaactac |
| | accaccaccagaggcaaaacgataactgatgcctgggttgaacctgctttcctgacactggtgc |
| | tgcctttgttgctgggtgtgaaagttgtcgccaccagtagccagattccactctatgaaagcgac |
| | aacgacttttgcgaatctgtcaagctggatggacccgccgatttctgggctttgctgcggctacc |
| | aacttccctgcatctccaggattggattggggggcgtgttcaaaatttagcagaaggcttgaacc |
| | gcttgatggttgcctactgcctgcatctggacagtcgtagcgctccaccagatgcgcgctggag |
| | agccttaaacggcagtgtccgagagatgacaacaaatgttctaaacattttttcacttgcagagg |
| | agggattacgtcgtgatggctctaaacgcgatgcaacagttgaagaaattaaccgctattggaa |
| | atacgcccagctgtgggcagagggagactctgatatgcaaaacaagttgaagattaccaaacg |
| | cttagtcgatgagtaccgccagttttacagagccagtgcgaacgaatccagccatacggttttgc |
| | tgccattttctaaggcactggaacacattctttcggtaccaaatgattgggacgatgaggaaatta |
| | ttgttcaaggggcaggacagttacaggccgcgttagatcggcaggaagtctacaaacgtcccc |
| | tcatgcttgacaaagagattccctttgaaattcgtcggatgaaggagtttgaggcaattcaaacct |
| | tcatgaccacctgcgtcattgacctatttggacaaatgtgtaaaggcgatcgcgcgctcctgcag |
| | gaacatcgcaaccgccttaaatcaggggtcgaatttgcctatcgtcagttagccctgcaagaga |
| | aacaagccaaagccaaccaaaaatctggagaacaatcatgagcattcttgaaaccttaaagcc |
| | agagttccacacagccttccctcgcctcgcctctggcaaatatgtccatttcttcatggtgcgtca |
| | cagccagtcgttccccgtctttcaaaccgatggcgttttgaacactgcccgtactcaggctgggtt |
| | aaatgcaggcgatgaaaaatccagacaggcaatcagtcgcctggtcatgttcaaacggaaaca |
| | ggtgacaccagaacgactcacaggacgggaactgctgcgatcgctgggcattaacggagtag |
| | ttgaaggggaagccaatttctgtgaatacaacggcgaaagatcctgtaaaaaatgccctgattg |
| | cattctctatggttttgcgattggggacagtggctcggaacgttccaaagtctactctgattcagc |
| | cttttccctgagtccttatgaagtttcccatcgcagttttaccttcaatgctccctttgaaggtggaac |
| | catgagtgagaagggtgaaatgcggagtgcgatcaacgaactggatcatgttttgccggaagtt |
| | gcttttccttgcgttgaaacactacgtgaccccacgctggaaggcttcatttatgtaatgggcaac |
| | ctgatccggactcggcgctatggagcgcaggagtcgcgtacagggacaatggcgaatcacat |
| | tattggcgttaccctatgtgatggcgagatcttcagcaatttgcactttacccaagccctctacgat |
| | gccctgaagggtgatttgagtgcgccaattagtgatttatgcagagaggcggcgatcgtggca |
| | gaaaacttgctaaatcaggaacctgtgcggaagatccaagttatttttggagagcagttgaacga |
| | actcctggctgaggtagcgggaatctgccaggatgaaactcgcttgcgaaacactgtgacagc |
| | cttgtaccagcaatcacaagcctatgccgagacctatggtgctgctgctaagaaaggcaaagg |
| | caaatcttaaggtggctgccaatggttatttatcgatgcaatctcacgttgcacgacaacgtattct |
| | tcgccactcgcgagatggggattctctacgaaacagagaagtatctacacaactgggcactca |
| | gctttgctctgtttgaagccacccacatccccaaaccctatcgcttgcagggtgcgatcgctcaa |
| | cgacccggttatctggaagctgacaacgagcaaaaccttttagttctgaatcacgcaggcatcta |
| | tgttttccctgctctaccacttcagtggtcctaccaagtcaataccttcaaagcggctcaagtgcct |
| | tactatggcaaatccaaacagttcggggataagggagctgatcgcaactacccgattaactacg |
| | gacgggcaaaagaactggcagttggcagcacctaccgaacgtatattcttgcccctgaaccaa |
| | tccagatcccccactggatacggttgggtaagtgggcagccaagattcaggtggacgcaaga |
| | cccatacctaactgtggaatccgacaacggtcaggagaatacacctgcgaccatccactaaatc |
| | cgatcgacctgtccggttcaacccgattgctgttatacaaccgcattgtcatgccacccgctagtc |
| | tcgtcagtcagtcgcgagtttctggggactactgggaaatttctaaaccatccaattggactgaat |
| | ttccggactggtcagacctgaaagacatacccaatcaagtttgcttacccagcggagtagcctat |
| | ggagccagtactgccattgccgcatcttaagtccaccgctctgcatatcctggtaattcatttagg |
| | cgttgccgaaagcggagccttgcctgttgaatccggtcgagccgtccatgctcaagttttggact |
| | ggttccgcatggcggatccggcaatttctgaggcaattcacaaaagccaggaatccccgctca |
| | gtctctcaggcctggtcgggaaacgccgtaaagtaggggtgcgggcaggagatgaattttact |
| | tccgaattgggttgttagatgggagtttgattgaaccactgctgagtggtttggagaagtggggc |
| | gacaagcccttttctctggcaaagtgtccctttgttttccgcagtatcaatatgttgccaggaactg |
| | acccctgggttggttcttccgattatgccttgcttacccaaaccccggctactttggataacctagc |
| | tctcaagctcctgtcgccaacaagcttcaagtcgaatgccgggcaaggaatccaaccctttcctt |
| | tgccagagtcagttttcggaaatttacatcgtcgctggaatgtctttgcaccaaaggaattgcagtt |
| | tccaaaaattcaatggagtgggctggtctctgattatgacctcaaaactcaaaagttaaggatgg |
| | aaaactcggttgagatcggcgcagtgggctgggtgcgctatcggtttccagatccagaacaag |
| | cgagaattgcaaccattctggctcactttgccttttttgctggagttgggcgtaagacagcaatgg |
| | ggatgggacaggtggtgttggacaaaaaaacgtagcctaggctgctgccaccgctgagcaat |
| | aactagcataaccccttggggcctctaaacgggtcttgaggggttttttgctgaaacctcaggca |
| | tttgagaagcacacggtcacactgcttccggtagtcaataaaccggtaaaccagcaatagacat |
| | aagcggctatttaacgaccctcgggatctcgacgctctcccttatgcgactcctgcattaggaaa |
| | ttaatacgactcactataggggaattgtgagcggataacaattcccctgtagaaataattttgttta |
| | actttaataaggagatataccatgcttgatgaaaaaagcttcgacaactggtgccaatccgttggt |
| | ctgagccaggcagcacgtgaagttgtagagcgtgtgcgatcctcttcaccctcccgtcgcgtac |
| | agagcggtagaggaaacgtctccggcaattaccctagccgcaaaatgggcgtcacaattcagt |
| | ttgagtcccatcgaaatgagcttgcccgcatctacgaactggagcatgaccccggcgttctgga |
| | atattacgatcaacctcccaaaatcgaacttgattatttgtcaaaaaatgggaggcgtgttcgtact |
| | caatacacacctgatttctttgttatccgtaatgactcagcaggttgggaggagtgtaaaacagag |
| | gatgaccttaagcaattagctgaggacagccctaaccgttaccaacgagctgaggacaatcgc |
| | tggcgatgtccacctgcagaagcctatgctcttccccttgagttgtattttcaggtacggtctagtg |
| | ctgaaattaactggattttccagcaaaactttgtatggctagaagactaccttggtactgaatcactt |
| | actatggatgagtcggttgcagatgcaatcatagccacagtcagagcaaacgagggtatcaca |
| | ctactcgaactgattcaacaattacaaggcacagatgctgatgctatctacatcctgattgcaacc |
| | gaacaaatttatgttgatttgatggctgaacggttagcgaacagcgatcgggcacgggtattttc |
| | cagtccagaaattgctcaagcctttcaactcattacgctggtcgaagcacctctggctaacagttc |
| | tctccatatagttcaggtgggcgttggcacaaccatacactgggatggtgaagtctgggagatt |
| | gttaatacaggctctaccatgacaggattgttgcgatcggacggtaagtttatcgagttgcccaat |
| | tctgcaattgatgaattgattcagaagggcaagattcaatcaatttcagcacaacaagcccctgat |
| | agcaaagcagaagttgaagaaatgcttcgctgtgctcgtccagccgacattaaagaagccaac |
| | cgtcgctacgaaatcattcagccttacctggaaaaggatgcaccagcttgtccaactcgaacaat |
| | tcggcgctggcgtaatcagtattggcaggcagaaaagctttacgggaatggctatgttggtttac |
| | taccaaatcatcagaagaaagggaattatttagttaaggttaatgaagtaactcaagcttttatgctt |
| | gagtttattgagaagcattacgaaaatctcaaacagcgtagtaagcttagagtttatcaggcttttg |
| | tggaggcatgtgaaagccacgaaccgaaactaaaccctccaagccgtactacattcacactag |
| | cgattcagaaacgagcaggacatcagcaaaccttgaaacgtgaaggacgaagagctgccatt |
| | cagaaggaggagttttactgggaactggagctaactactcctcgacacggcgatcgccccttc |
| | gagatcgttcatattgatcacactcaactcgatattgagcttatcagttctttgaactccctggccca |
| | atgcaaccctacatctgttgatgccacttacaatttgggtcgtccttgggcaacgttcatggttgat |
| | gcttacagccgccgtttgctagcagtttacataacctatgaagaacccagctatcgttcctgcatg |
| | atggtgctccgtatttgcgtacagcgatttaagcgttttcctcagacgatcgtggtagataatggtg |
| | cagaatttcacagccattactttgaacaactactggcaacatatcaatgcacaaagaagcacag |
| | gccacctgcaaaagctcgctttggtgcagttgttgaacgattatttggcactgcaaataagcaatt |
| | aattcatgagttgcagggaaatacccaaattactcgcaagcatcgccaagttactcaatctgttca |
| | tccccaaaaattggctctttggacgttaggcgatctctacgaatcactatgtgaatgggcttacaa |
| | agtttatgctgaccgccagcaccctgcgctagggcaaagccctaatgctgcattcgctcaaggg |
| | ctggcgttgggaggtagtcgcatccatcgccgagtagagtatgaccaagtcttccgtatcttgac |
| | attgcccgcaccggaaagaggacaacgcaaagttcagccaggacaagggataaaacttagc |
| | aatatctactactggtcaaatgcctttcgagacccagaaattgaacaatcgatggttgaagttcgtt |
| | atgatccctttgatgccagcattgcctatgcactggtgcgaaaacaatgggtccggtgtatttcct |
| | cctactatcaacagtttcaaaatcgttcagaaaaggaaattcgtttaataactgctgagttgattaa |
| | gcaaaagcgtgaccagggaagagatacaaatgttacggataaagaattggcgcagtttttaag |
| | ctctacagaaatgaaagaaggcaaattcttggagcaacgcttgcggactacagaaaacagggt |
| | agttattcagaatagtacagctacagaagacgatccccccatcactaattctcagaattcttacga |
| | gccatcgcttatgctgccaagtgaggatgagttggatgatcaggaagactggttaaataccggc |
| | aaagctgaggcagaattagaattctatggagagtactagcgatgcaacctcaaataagcttccc |
| | aaaagagttactgcttcaaccaattcctgatcgaattgcctatttcaaagattacacaatggcacat |
| | ccgaagcttatagaagcagaccggaagttaatgcatgctattcgtgaacctggaggagctgctc |
| | ttatctttgtctttggtcctaccggagttggaaagtctactctattgaaacgaattactcaaaagctg |
| | cttaaagaagcattgtctttgatggagactgacaaaggctatattcctattgcgggggtagagata |
| | aaaactccagaggatagtaactttgattggaacgatttttatgttcgtgctcttatggcacttaatga |
| | gccaatgatcgagaaaaaaatcaaccgtacaggtgcaaaactaaaattacggttggcacttgaa |
| | tctgcccttcgtaatcgccgtcctgatgctttttacatagacgaagctcaaaattttggtaaggtag |
| | ccagtgggcgaaaattgcaagatcaaacagattgcattaaatctcttgcaaatctcgctcaaact |
| | cggtttatcttatgtgggatttatgaacttttaccactacgaaatctaagtgggcagctatgccgcc |
| | gtagccatagtattcactttccacgttattcggcagattctgagcaggatctaaaggcctttaaaag |
| | tgcgttattaacactacaaaatcaactccctcttcccgaaccacctgatttagtggcgcattggga |
| | attttgttatgagagaactatcggctgtattggtattttgaaagatttgctccttcggactctaagcg |
| | cgattttagtacgggatggagacaaagttttgacattgaagcccaaggatttagaacaacgtgca |
| | tggtctttagaagaatgtttggttatgctgagagaggcaaaggaaggagagcaacaacttactg |
| | acaaatcgggcttacatgaggaattacggacagctttagggtttaatgcagctacgtccaagca |
| | gtctgccaaatcatccaagtcaacatcaagcaaaaaagcaggcagtgttggaaagccttcacct |
| | cgtcgtcgttcagttggaatagatcaagatgccagctaatagcagctccaattacgaaagaaaa |
| | atattttcaactagaatcgagcttaataaattgtccaattaagtatgctcaacagcagttcttaaagc |
| | tcttcttcaattaacacaaaaacgcccagttttaacttaagtcgaacagaaagtaatcgtattgtac |
| | acggccgcataatcgaaattaatacgactcactataggggaattgtgagcggataacaattccc |
| | catcttagtatattagttaagtataagaaggagatatacatatggcagatctcaattggatatcggc |
| | cggccacgcgatcgctgacgtcggtaccctcgagtctggtaaagaaaccgctgctgcgaaatt |
| | tgaacgccagcacatggactcgtctactagcgcagcttaattaacctaggctgctgccaccgct |
| | gagcaataactagcataaccccttggggcctctaaacgggtcttgaggggttttttgctgaaacc |
| | tcaggcatttgagaagcacacggtcacactgcttccggtagtgccctgaaccgacgaccgggt |
| | catcgtggccggatcttgcggcccctcggcttgaacgaattgttagacattatttgccgactacct |
| | tggtgatctcgcctttcacgtagtggacaaattcttccaactgatctgcgcgcgaggccaagcga |
| | tcttcttcttgtccaagataagcctgtctagcttcaagtatgacgggctgatactgggccggcagg |
| | cgctccattgcccagtcggcagcgacatccttcggcgcgattttgccggttactgcgctgtacca |
| | aatgcgggacaacgtaagcactacatttcgctcatcgccagcccagtcgggcggcgagttcca |
| | tagcgttaaggtttcatttagcgcctcaaatagatcctgttcaggaaccggatcaaagagttcctc |
| | cgccgctggacctaccaaggcaacgctatgttctcttgcttttgtcagcaagatagccagatcaa |
| | tgtcgatcgtggctggctcgaagatacctgcaagaatgtcattgcgctgccattctccaaattgca |
| | gttcgcgcttagctggataacgccacggaatgatgtcgtcgtgcacaacaatggtgacttctaca |
| | gcgcggagaatctcgctctctccaggggaagccgaagtttccaaaaggtcgttgatcaaagct |
| | cgccgcgttgtttcatcaagccttacggtcaccgtaaccagcaaatcaatatcactgtgtggcttc |
| | aggccgccatccactgcggagccgtacaaatgtacggccagcaacgtcggttcgagatggcg |
| | ctcgatgacgccaactacctctgatagttgagtcgatacttcggcgatcaccgcttccctcatact |
| | cttcctttttcaatattattgaagcatttatcagggttattgtctcatgagcggatacatatttgaatg |
| | tatttagaaaaataaacaaatagctagctcactcggtcgctacgctccgggcgtgagactgcggc |
| | gggcgctgcggacacatacaaagttacccacagattccgtggataagcaggggactaacatgt |
| | gaggcaaaacagcagggccgcgccggtggcgtttttccataggctccgccctcctgccagag |
| | ttcacataaacagacgcttttccggtgcatctgtgggagccgtgaggctcaaccatgaatctgac |
| | agtacgggcgaaacccgacaggacttaaagatccccaccgtttccgggggtcgctccctctt |
| | gcgctctcctgttccgaccctgccgtttaccggatacctgttccgcctttctcccttacgggaagt |
| | gtggcgctttctcatagctcacacactggtatctcggctcggtgtaggtcgttcgctccaagctg |
| | ggctgtaagcaagaactccccgttcagcccgactgctgcgccttatccggtaactgttcacttga |
| | gtccaacccggaaaagcacggtaaaacgccactggcagcagccattggtaactgggagttcg |
| | cagaggatttgtttagctaaacacgcggttgctcttgaagtgtgcgccaaagtccggctacactg |
| | gaaggacagatttggttgctgtgctctgcgaaagccagttaccacggttaagcagttccccaact |
| | gacttaaccttcgatcaaaccacctccccaggtggttttttcgtttacagggcaaaagattacgcg |
| | cagaaaaaaaggatctcaagaagatcctttgatcttttctactgaaccgctctagatttcagtgcaa |
| | tttatctcttcaaatgtagcacctgaagtcagccccatacgatataagttgtaattctcatgttagtca |
| | tgccccgcgcccaccggaaggagctgactgggttgaaggctctcaagggcatcggtcgagat |
| | cccggtgcctaatgagtgagctaacttacattaattgcgttgcgctcactgcccgctttccagtcg |
| | ggaaacctgtcgtgccagctgcattaatgaatcggccaacgcgcggggagaggcggtttgcg |
| | tattgggcgccagggtggtttttcttttcaccagtgagacgggcaacagctgattgcccttcacc |
| | gcctggccctgagagagttgcagcaagcggtccacgctggtttgccccagcaggcgaaaatc |
| | ctgtttgatggtggttaacggcgggatataacatgagctgtcttcggtatcgtcgtatcccactac |
| | cgagatgtccgcaccaacgcgcagcccggactcggtaatggcgcgcattgcgcccagcgcc |
| | atctgatcgttggcaaccagcatcgcagtgggaacgatgccctcattcagcatttgcatggtttgt |
| | tgaaaaccggacatggcactccagtcgccttcccgttccgctatcggctgaatttgattgcgagt |
| | gagatatttatgccagccagccagacgcagacgcgccgagacagaacttaatgggcccgcta |
| | acagcgcgatttgctggtgacccaatgcgaccagatgctccacgcccagtcgcgtaccgtcttc |
| | atgggagaaaataatactgttgatgggtgtctggtcagagacatcaagaaataacgccggaac |
| | attagtgcaggcagcttccacagcaatggcatcctggtcatccagcggatagttaatgatcagc |
| | ccactgacgcgttgcgcgagaagattgtgcaccgccgctttacaggcttcgacgccgcttcgtt |
| | ctaccatcgacaccaccacgctggcacccagttgatcggcgcgagatttaatcgccgcgacaa |
| | tttgcgacggcgcgtgcagggccagactggaggtggcaacgccaatcagcaacgactgtttg |
| | cccgccagttgttgtgccacgcggttgggaatgtaattcagctccgccatcgccgcttccactttt |
| | tcccgcgttttcgcagaaacgtggctggcctggttcaccacgcgggaaacggtctgataagag |
| | acaccggcatactctgcgacatcgtataacgttactggtttcacattcaccaccctgaattgactct |
| | cttccgggcgctatcatgccataccgcgaaaggttttgcgccattcgatggtgtccgggatctcg |
| | acgctctcccttatgcgactcctgcattaggaaattaatacgactcactata (SEQ ID NO: |
| | 96) |
| pHelper(CyCAST)_ | ggggaattgtgagcggataacaattcccctgtagaaataattttgtttaactttaataaggagatat |
| Shuffled- | accatggtttccaagcctgaatcccggtctacgggactgaaacaccatctatgtatagctacgat |
| spacer_ΔTniQΔTnsD | ggaatagtaaagggtttccaagcctgaatcccggtctacgggactgaaacgatccgaattcga |
| | gctcggcgcgcctgcaggtcgacaagcttgcggccgcataatgcttaagtcgaacagaaagta |
| | atcgtattgtacacggccgcataatcgaaattaatacgactcactataggggaattgtgagcgga |
| | taacaattccccatcttagtatattagttaagtataagaaggagatatacatatggctaccctactcc |
| | aaaccctcctgttacgaacgttgtcacccgaggcggatccagttctctatgtctacattgagacg |
| | gtactacctgcaatggaacgggaatttggcttgattccagcccttggtggctcggaggaagcac |
| | actttcatctgctgaggcgacggggaaacaaatatgcggcggaaaattccaagcggtacgcca |
| | gtcgctctgatcaaagcctgctggttcatgtgctcaatgccttgctgaccgcctggaatttgttgc |
| | cattcctaagctccacatttgaactatcagatgatgagaaacggctgctctgcctggggctgacc |
| | ctacacgattacaacaagtggtgccagggtgaggaggaagattcgccgattgcatgcgaagta |
| | gcagagattcttgagctttgtcaaacattgggagagcggttgaactttgacggattctgggcaga |
| | gtggcgggagtatttgacagagattgcctaccttgcccaaaacacccagggaaaagttggaac |
| | aaatctacccaaagcgaattggggcagcttcaaaattctagatacccgtcgtctggattcgcccc |
| | tacgatggctactcgcttttggcgatgtcgcagtccatctcagcgatccatctgacatcgcaatga |
| | aaacggggggagatcgcctgcgggaacatctgcgctttctaggtattcgtcgcaagctggtcta |
| | ccatcgcctgcgcaactgtacagggctattaaccaacggaattcataatgcggtaatgcattttg |
| | ctagagaactggattggcaacccatcctcttctttgctcagggtgttgtttatctcgctcctcaggg |
| | tgctggcgttccggatcgcttagaacttcaggaatttctctgggagcaggtcagcaaccgattag |
| | ccaccagcatgttgcggggagaggttggtttcaaacgggatggcaagggactcaaggtggct |
| | ccccaaaccctggaactgttctcccctgctcagttgattcggaacttgcccggtgttgtccaatctt |
| | atgtcggaaacgctaaatctcctgcgactccaaaacgacttgagaagctggaactcagttcaac |
| | agagcgagagtttttggctaaaggtgcagatttaagagcagatcgcattgccgagtttatctttct |
| | ggcgcaaaaagagtttttcggagataaaccagaatttatcacttggatgcttgaaactctggggg |
| | ttcaaactgctatcacttcggagcagacgcagcttcaatccggtggtgtgaactggggatggtat |
| | cacgcagcagcctattacgtcgcgagcaatgccacgctggatttggaagagatttcggagaaa |
| | cttcaggaatttgccgagcagttggcaacctgggggaggaaaacaatttcttcccagagcatc |
| | gcagcccaacccatgacatctttttcaactatctgactcaatacctggaagttcaggggtgggag |
| | cagcaggagcagtcctttcagcttgaactggcaggctatgttggcgcaaagactaaaactgcca |
| | gacaaccgatttgctccctcagttccggtgaatttgcttcggaagaccagatggattctgttgtcct |
| | gttcaaaccgcagcagtacagcaacaagaatcctctcggtggacggcagatcaagcggggg |
| | atttccaaaatctggtccctggaaatgctgctcaggcaggctctttggtctgcgccagcaggcaa |
| | actagaggaacaacaacccattttcctttacatcttcccggcatacgtttactcgccccaggttgct |
| | gctgctgtccggttactggtgaatgatctgaagcgtgtcaacctctgggaagtccgtaaacactg |
| | gttggagaatggcatggatgctcaggcgttgcgatcgtttccctggctagtcggtgacaaaaac |
| | gattcgggtaactctgctccatccagatactctggtgaagacttgcctttcatggcaatgaactac |
| | accaccaccagaggcaaaacgataactgatgcctgggttgaacctgctttcctgacactggtgc |
| | tgcctttgttgctgggtgtgaaagttgtcgccaccagtagccagattccactctatgaaagcgac |
| | aacgacttttgcgaatctgtcaagctggatggacccgccgatttctgggctttgctgcggctacc |
| | aacttccctgcatctccaggattggattggggggcgtgttcaaaatttagcagaaggcttgaacc |
| | gcttgatggttgcctactgcctgcatctggacagtcgtagcgctccaccagatgcgcgctggag |
| | agccttaaacggcagtgtccgagagatgacaacaaatgttctaaacattttttcacttgcagagg |
| | agggattacgtcgtgatggctctaaacgcgatgcaacagttgaagaaattaaccgctattggaa |
| | atacgcccagctgtgggcagagggagactctgatatgcaaaacaagttgaagattaccaaacg |
| | cttagtcgatgagtaccgccagttttacagagccagtgcgaacgaatccagccatacggttttgc |
| | tgccattttctaaggcactggaacacattctttcggtaccaaatgattgggacgatgaggaaatta |
| | ttgttcaaggggcaggacagttacaggccgcgttagatcggcaggaagtctacaaacgtcccc |
| | tcatgcttgacaaagagattccctttgaaattcgtcggatgaaggagtttgaggcaattcaaacct |
| | tcatgaccacctgcgtcattgacctatttggacaaatgtgtaaaggcgatcgcgcgctcctgcag |
| | gaacatcgcaaccgccttaaatcaggggtcgaatttgcctatcgtcagttagccctgcaagaga |
| | aacaagccaaagccaaccaaaaatctggagaacaatcatgagcattcttgaaaccttaaagcc |
| | agagttccacacagccttccctcgcctcgcctctggcaaatatgtccatttcttcatggtgcgtca |
| | cagccagtcgttccccgtctttcaaaccgatggcgttttgaacactgcccgtactcaggctgggtt |
| | aaatgcaggcgatgaaaaatccagacaggcaatcagtcgcctggtcatgttcaaacggaaaca |
| | ggtgacaccagaacgactcacaggacgggaactgctgcgatcgctgggcattaacggagtag |
| | ttgaaggggaagccaatttctgtgaatacaacggcgaaagatcctgtaaaaaatgccctgattg |
| | cattctctatggttttgcgattggggacagtggctcggaacgttccaaagtctactctgattcagc |
| | cttttccctgagtccttatgaagtttcccatcgcagttttaccttcaatgctccctttgaaggtggaac |
| | catgagtgagaagggtgaaatgcggagtgcgatcaacgaactggatcatgttttgccggaagtt |
| | gcttttccttgcgttgaaacactacgtgaccccacgctggaaggcttcatttatgtaatgggcaac |
| | ctgatccggactcggcgctatggagcgcaggagtcgcgtacagggacaatggcgaatcacat |
| | tattggcgttaccctatgtgatggcgagatcttcagcaatttgcactttacccaagccctctacgat |
| | gccctgaagggtgatttgagtgcgccaattagtgatttatgcagagaggcggcgatcgtggca |
| | gaaaacttgctaaatcaggaacctgtgcggaagatccaagttatttttggagagcagttgaacga |
| | actcctggctgaggtagcgggaatctgccaggatgaaactcgcttgcgaaacactgtgacagc |
| | cttgtaccagcaatcacaagcctatgccgagacctatggtgctgctgctaagaaaggcaaagg |
| | caaatcttaaggtggctgccaatggttatttatcgatgcaatctcacgttgcacgacaacgtattct |
| | tcgccactcgcgagatggggattctctacgaaacagagaagtatctacacaactgggcactca |
| | gctttgctctgtttgaagccacccacatccccaaaccctatcgcttgcagggtgcgatcgctcaa |
| | cgacccggttatctggaagctgacaacgagcaaaaccttttagttctgaatcacgcaggcatcta |
| | tgttttccctgctctaccacttcagtggtcctaccaagtcaataccttcaaagcggctcaagtgcct |
| | tactatggcaaatccaaacagttcggggataagggagctgatcgcaactacccgattaactacg |
| | gacgggcaaaagaactggcagttggcagcacctaccgaacgtatattcttgcccctgaaccaa |
| | tccagatcccccactggatacggttgggtaagtgggcagccaagattcaggtggacgcaaga |
| | cccatacctaactgtggaatccgacaacggtcaggagaatacacctgcgaccatccactaaatc |
| | cgatcgacctgtccggttcaacccgattgctgttatacaaccgcattgtcatgccacccgctagtc |
| | tcgtcagtcagtcgcgagtttctggggactactgggaaatttctaaaccatccaattggactgaat |
| | ttccggactggtcagacctgaaagacatacccaatcaagtttgcttacccagcggagtagcctat |
| | ggagccagtactgccattgccgcatcttaagtccaccgctctgcatatcctggtaattcatttagg |
| | cgttgccgaaagcggagccttgcctgttgaatccggtcgagccgtccatgctcaagttttggact |
| | ggttccgcatggcggatccggcaatttctgaggcaattcacaaaagccaggaatccccgctca |
| | gtctctcaggcctggtcgggaaacgccgtaaagtaggggtgcgggcaggagatgaattttact |
| | tccgaattgggttgttagatgggagtttgattgaaccactgctgagtggtttggagaagtggggc |
| | gacaagcccttttctctggcaaagtgtccctttgttttccgcagtatcaatatgttgccaggaactg |
| | acccctgggttggttcttccgattatgccttgcttacccaaaccccggctactttggataacctagc |
| | tctcaagctcctgtcgccaacaagcttcaagtcgaatgccgggcaaggaatccaaccctttcctt |
| | tgccagagtcagttttcggaaatttacatcgtcgctggaatgtctttgcaccaaaggaattgcagtt |
| | tccaaaaattcaatggagtgggctggtctctgattatgacctcaaaactcaaaagttaaggatgg |
| | aaaactcggttgagatcggcgcagtgggctgggtgcgctatcggtttccagatccagaacaag |
| | cgagaattgcaaccattctggctcactttgccttttttgctggagttgggcgtaagacagcaatgg |
| | ggatgggacaggtggtgttggacaaaaaaacgtagcctaggctgctgccaccgctgagcaat |
| | aactagcataaccccttggggcctctaaacgggtcttgaggggttttttgctgaaacctcaggca |
| | tttgagaagcacacggtcacactgcttccggtagtcaataaaccggtaaaccagcaatagacat |
| | aagcggctatttaacgaccctcgggatctcgacgctctcccttatgcgactcctgcattaggaaa |
| | ttaatacgactcactataggggaattgtgagcggataacaattcccctgtagaaataattttgttta |
| | actttaataaggagatataccatgcttgatgaaaaaagcttcgacaactggtgccaatccgttggt |
| | ctgagccaggcagcacgtgaagttgtagagcgtgtgcgatcctcttcaccctcccgtcgcgtac |
| | agagcggtagaggaaacgtctccggcaattaccctagccgcaaaatgggcgtcacaattcagt |
| | ttgagtcccatcgaaatgagcttgcccgcatctacgaactggagcatgaccccggcgttctgga |
| | atattacgatcaacctcccaaaatcgaacttgattatttgtcaaaaaatgggaggcgtgttcgtact |
| | caatacacacctgatttctttgttatccgtaatgactcagcaggttgggaggagtgtaaaacagag |
| | gatgaccttaagcaattagctgaggacagccctaaccgttaccaacgagctgaggacaatcgc |
| | tggcgatgtccacctgcagaagcctatgctcttccccttgagttgtattttcaggtacggtctagtg |
| | ctgaaattaactggattttccagcaaaactttgtatggctagaagactaccttggtactgaatcactt |
| | actatggatgagtcggttgcagatgcaatcatagccacagtcagagcaaacgagggtatcaca |
| | ctactcgaactgattcaacaattacaaggcacagatgctgatgctatctacatcctgattgcaacc |
| | gaacaaatttatgttgatttgatggctgaacggttagcgaacagcgatcgggcacgggtattttc |
| | cagtccagaaattgctcaagcctttcaactcattacgctggtcgaagcacctctggctaacagttc |
| | tctccatatagttcaggtgggcgttggcacaaccatacactgggatggtgaagtctgggagatt |
| | gttaatacaggctctaccatgacaggattgttgcgatcggacggtaagtttatcgagttgcccaat |
| | tctgcaattgatgaattgattcagaagggcaagattcaatcaatttcagcacaacaagcccctgat |
| | agcaaagcagaagttgaagaaatgcttcgctgtgctcgtccagccgacattaaagaagccaac |
| | cgtcgctacgaaatcattcagccttacctggaaaaggatgcaccagcttgtccaactcgaacaat |
| | tcggcgctggcgtaatcagtattggcaggcagaaaagctttacgggaatggctatgttggtttac |
| | taccaaatcatcagaagaaagggaattatttagttaaggttaatgaagtaactcaagcttttatgctt |
| | gagtttattgagaagcattacgaaaatctcaaacagcgtagtaagcttagagtttatcaggcttttg |
| | tggaggcatgtgaaagccacgaaccgaaactaaaccctccaagccgtactacattcacactag |
| | cgattcagaaacgagcaggacatcagcaaaccttgaaacgtgaaggacgaagagctgccatt |
| | cagaaggaggagttttactgggaactggagctaactactcctcgacacggcgatcgccccttc |
| | gagatcgttcatattgatcacactcaactcgatattgagcttatcagttctttgaactccctggccca |
| | atgcaaccctacatctgttgatgccacttacaatttgggtcgtccttgggcaacgttcatggttgat |
| | gcttacagccgccgtttgctagcagtttacataacctatgaagaacccagctatcgttcctgcatg |
| | atggtgctccgtatttgcgtacagcgatttaagcgttttcctcagacgatcgtggtagataatggtg |
| | cagaatttcacagccattactttgaacaactactggcaacatatcaatgcacaaagaagcacag |
| | gccacctgcaaaagctcgctttggtgcagttgttgaacgattatttggcactgcaaataagcaatt |
| | aattcatgagttgcagggaaatacccaaattactcgcaagcatcgccaagttactcaatctgttca |
| | tccccaaaaattggctctttggacgttaggcgatctctacgaatcactatgtgaatgggcttacaa |
| | agtttatgctgaccgccagcaccctgcgctagggcaaagccctaatgctgcattcgctcaaggg |
| | ctggcgttgggaggtagtcgcatccatcgccgagtagagtatgaccaagtcttccgtatcttgac |
| | attgcccgcaccggaaagaggacaacgcaaagttcagccaggacaagggataaaacttagc |
| | aatatctactactggtcaaatgcctttcgagacccagaaattgaacaatcgatggttgaagttcgtt |
| | atgatccctttgatgccagcattgcctatgcactggtgcgaaaacaatgggtccggtgtatttcct |
| | cctactatcaacagtttcaaaatcgttcagaaaaggaaattcgtttaataactgctgagttgattaa |
| | gcaaaagcgtgaccagggaagagatacaaatgttacggataaagaattggcgcagtttttaag |
| | ctctacagaaatgaaagaaggcaaattcttggagcaacgcttgcggactacagaaaacagggt |
| | agttattcagaatagtacagctacagaagacgatccccccatcactaattctcagaattcttacga |
| | gccatcgcttatgctgccaagtgaggatgagttggatgatcaggaagactggttaaataccggc |
| | aaagctgaggcagaattagaattctatggagagtactagcgatgcaacctcaaataagcttccc |
| | aaaagagttactgcttcaaccaattcctgatcgaattgcctatttcaaagattacacaatggcacat |
| | ccgaagcttatagaagcagaccggaagttaatgcatgctattcgtgaacctggaggagctgctc |
| | ttatctttgtctttggtcctaccggagttggaaagtctactctattgaaacgaattactcaaaagctg |
| | cttaaagaagcattgtctttgatggagactgacaaaggctatattcctattgcgggggtagagata |
| | aaaactccagaggatagtaactttgattggaacgatttttatgttcgtgctcttatggcacttaatga |
| | gccaatgatcgagaaaaaaatcaaccgtacaggtgcaaaactaaaattacggttggcacttgaa |
| | tctgcccttcgtaatcgccgtcctgatgctttttacatagacgaagctcaaaattttggtaaggtag |
| | ccagtgggcgaaaattgcaagatcaaacagattgcattaaatctcttgcaaatctcgctcaaact |
| | cggtttatcttatgtgggatttatgaacttttaccactacgaaatctaagtgggcagctatgccgcc |
| | gtagccatagtattcactttccacgttattcggcagattctgagcaggatctaaaggcctttaaaag |
| | tgcgttattaacactacaaaatcaactccctcttcccgaaccacctgatttagtggcgcattggga |
| | attttgttatgagagaactatcggctgtattggtattttgaaagatttgctccttcggactctaagcg |
| | cgattttagtacgggatggagacaaagttttgacattgaagcccaaggatttagaacaacgtgca |
| | tggtctttagaagaatgtttggttatgctgagagaggcaaaggaaggagagcaacaacttactg |
| | acaaatcgggcttacatgaggaattacggacagctttagggtttaatgcagctacgtccaagca |
| | gtctgccaaatcatccaagtcaacatcaagcaaaaaagcaggcagtgttggaaagccttcacct |
| | cgtcgtcgttcagttggaatagatcaagatgccagctaatagcagctccaattacgaaagaaaa |
| | atattttcaactagaatcgagcttaataaattgtccaattaagtatgctcaacagcagttcttaaagc |
| | tcttcttcaattaacacaaaaacgcccagttttaacttaagtcgaacagaaagtaatcgtattgtac |
| | acggccgcataatcgaaattaatacgactcactataggggaattgtgagcggataacaattccc |
| | catcttagtatattagttaagtataagaaggagatatacatatggcagatctcaattggatatcggc |
| | cggccacgcgatcgctgacgtcggtaccctcgagtctggtaaagaaaccgctgctgcgaaatt |
| | tgaacgccagcacatggactcgtctactagcgcagcttaattaacctaggctgctgccaccgct |
| | gagcaataactagcataaccccttggggcctctaaacgggtcttgaggggttttttgctgaaacc |
| | tcaggcatttgagaagcacacggtcacactgcttccggtagtgccctgaaccgacgaccgggt |
| | catcgtggccggatcttgcggcccctcggcttgaacgaattgttagacattatttgccgactacct |
| | tggtgatctcgcctttcacgtagtggacaaattcttccaactgatctgcgcgcgaggccaagcga |
| | tcttcttcttgtccaagataagcctgtctagcttcaagtatgacgggctgatactgggccggcagg |
| | cgctccattgcccagtcggcagcgacatccttcggcgcgattttgccggttactgcgctgtacca |
| | aatgcgggacaacgtaagcactacatttcgctcatcgccagcccagtcgggcggcgagttcca |
| | tagcgttaaggtttcatttagcgcctcaaatagatcctgttcaggaaccggatcaaagagttcctc |
| | cgccgctggacctaccaaggcaacgctatgttctcttgcttttgtcagcaagatagccagatcaa |
| | tgtcgatcgtggctggctcgaagatacctgcaagaatgtcattgcgctgccattctccaaattgca |
| | gttcgcgcttagctggataacgccacggaatgatgtcgtcgtgcacaacaatggtgacttctaca |
| | gcgcggagaatctcgctctctccaggggaagccgaagtttccaaaaggtcgttgatcaaagct |
| | cgccgcgttgtttcatcaagccttacggtcaccgtaaccagcaaatcaatatcactgtgtggcttc |
| | aggccgccatccactgcggagccgtacaaatgtacggccagcaacgtcggttcgagatggcg |
| | ctcgatgacgccaactacctctgatagttgagtcgatacttcggcgatcaccgcttccctcatact |
| | cttcctttttcaatattattgaagcatttatcagggttattgtctcatgagcggatacatatttgaatg |
| | tatttagaaaaataaacaaatagctagctcactcggtcgctacgctccgggcgtgagactgcggc |
| | gggcgctgcggacacatacaaagttacccacagattccgtggataagcaggggactaacatgt |
| | gaggcaaaacagcagggccgcgccggtggcgtttttccataggctccgccctcctgccagag |
| | ttcacataaacagacgcttttccggtgcatctgtgggagccgtgaggctcaaccatgaatctgac |
| | agtacgggcgaaacccgacaggacttaaagatccccaccgtttccggcgggtcgctccctctt |
| | gcgctctcctgttccgaccctgccgtttaccggatacctgttccgcctttctcccttacgggaagt |
| | gtggcgctttctcatagctcacacactggtatctcggctcggtgtaggtcgttcgctccaagctg |
| | ggctgtaagcaagaactccccgttcagcccgactgctgcgccttatccggtaactgttcacttga |
| | gtccaacccggaaaagcacggtaaaacgccactggcagcagccattggtaactgggagttcg |
| | cagaggatttgtttagctaaacacgcggttgctcttgaagtgtgcgccaaagtccggctacactg |
| | gaaggacagatttggttgctgtgctctgcgaaagccagttaccacggttaagcagttccccaact |
| | gacttaaccttcgatcaaaccacctccccaggtggttttttcgtttacagggcaaaagattacgcg |
| | cagaaaaaaaggatctcaagaagatcctttgatcttttctactgaaccgctctagatttcagtgcaa |
| | tttatctcttcaaatgtagcacctgaagtcagccccatacgatataagttgtaattctcatgttagtca |
| | tgccccgcgcccaccggaaggagctgactgggttgaaggctctcaagggcatcggtcgagat |
| | cccggtgcctaatgagtgagctaacttacattaattgcgttgcgctcactgcccgctttccagtcg |
| | ggaaacctgtcgtgccagctgcattaatgaatcggccaacgcgcggggagaggcggtttgcg |
| | tattgggcgccagggtggtttttcttttcaccagtgagacgggcaacagctgattgcccttcacc |
| | gcctggccctgagagagttgcagcaagcggtccacgctggtttgccccagcaggcgaaaatc |
| | ctgtttgatggtggttaacggcgggatataacatgagctgtcttcggtatcgtcgtatcccactac |
| | cgagatgtccgcaccaacgcgcagcccggactcggtaatggcgcgcattgcgcccagcgcc |
| | atctgatcgttggcaaccagcatcgcagtgggaacgatgccctcattcagcatttgcatggtttgt |
| | tgaaaaccggacatggcactccagtcgccttcccgttccgctatcggctgaatttgattgcgagt |
| | gagatatttatgccagccagccagacgcagacgcgccgagacagaacttaatgggcccgcta |
| | acagcgcgatttgctggtgacccaatgcgaccagatgctccacgcccagtcgcgtaccgtcttc |
| | atgggagaaaataatactgttgatgggtgtctggtcagagacatcaagaaataacgccggaac |
| | attagtgcaggcagcttccacagcaatggcatcctggtcatccagcggatagttaatgatcagc |
| | ccactgacgcgttgcgcgagaagattgtgcaccgccgctttacaggcttcgacgccgcttcgtt |
| | ctaccatcgacaccaccacgctggcacccagttgatcggcgcgagatttaatcgccgcgacaa |
| | tttgcgacggcgcgtgcagggccagactggaggtggcaacgccaatcagcaacgactgtttg |
| | cccgccagttgttgtgccacgcggttgggaatgtaattcagctccgccatcgccgcttccactttt |
| | tcccgcgttttcgcagaaacgtggctggcctggttcaccacgcgggaaacggtctgataagag |
| | acaccggcatactctgcgacatcgtataacgttactggtttcacattcaccaccctgaattgactct |
| | cttccgggcgctatcatgccataccgcgaaaggttttgcgccattcgatggtgtccgggatctcg |
| | acgctctcccttatgcgactcctgcattaggaaattaatacgactcactata (SEQ ID NO: |
| | 97) |
| pDonor(CyCAST)_ | tcgcgcgtttcggtgatgacggtgaaaacctctgacacatgcagctcccggagactgtcacag |
| 0.5 kb-cargo | cttgtctgtaagcggatgccgggagcagacaagcccgtcagggcgcgtcagcgggtgttggc |
| | gggtgtcggggctggcttaactatgcggcatcagagcagattgtactgagagtgcaccatatgc |
| | ggtgtgaaataccgcacagatgcgtaaggagaaaataccgcatcaggcgccattcgccattca |
| | ggctgcgcaactgttgggaagggcgatcggtgcgggcctcttcgctattacgccagctggcga |
| | aagggggatgtgctgcaaggcgattaagttgggtaacgccagggttttcccagtcacgacgtt |
| | gtaaaacgacggccagtgaattgacgcgtattgggattaaaaacgttcaaccccataaatcatg |
| | catgttgtgactgcaaccataagttgtctatttcggacactttatgcttacaattttggctggacagc |
| | ttatgcttacaattttggctggacagatagagcgaaagcaataggcaagacgattatatttctttgc |
| | ctataaagtaggcctattgatggatgaccctccaaccgggttcgtgagtggcttccctgccgcg |
| | gccgccgcgggcgcgggccgctcgctctacggtgggcccgagctcgtgttccccgaggcca |
| | tgaaccaccccgcgctgaccgtgcatccggcgcaccagctgggcgcctccccgctgcagcc |
| | cccgcactccttcttcggcgcccagcaccgggaccctctccatttctacccctgggtcctgcgg |
| | aaccgcttcttcggccaccgcttccagggtgagtgtccacgctgtgcccgccgaggcggccg |
| | gccggcgcccgtgctgcggcgatgcgggggaggctcgggggcgcgcggggctgtttagaa |
| | gttactgccgggaaggctgcaggtccgcggaggtagattcccaggcagggaagagctgtgc |
| | ggcatccacccgcgccttcgccgcgtaggtctccctcccaggaaagcaggtggagatattggt |
| | gcccttctagagtcagaagtcattccttcggagcttttccaggattaaaagcatacttgctcttggc |
| | aaataaccctaaggcaacacaacggctgctccaagttctaacttttggacaacttatgctttcgatt |
| | gttaacatttgggcaacttatgctttcagttttttattttgcaaagtgaatattgacgatgactcaagt |
| | tgtccacacccatgagtggacaacttatgctttcaaaatcggacaacttatggttgcagtcacacat |
| | gttgtgactgggcaagtgagatgatagtcacctgatttgcttctccctcactgcttccttagcctaa |
| | aatcccaatggcgcgccgagcttggcgtaatcatggtcatagctgtttcctgtgtgaaattgttat |
| | ccgctcacaattccacacaacatacgagccggaagcataaagtgtaaagcctggggtgcctaa |
| | tgagtgagctaactcacattaattgcgttgcgctcactgcccgctttccagtcgggaaacctgtcg |
| | tgccagctgcattaatgaatcggccaacgcgcggggagaggcggtttgcgtattgggcgctgt |
| | tccgcttcctcgctcactgactcgctgcgctcggtcgttcggctgcggcgagcggtatcagctc |
| | actcaaaggcggtaatacggttatccacagaatcaggggataacgcaggaaagaacatgtga |
| | gcaaaaggccagcaaaaggccaggaaccgtaaaaaggccgcgttgctggcgtttttccatag |
| | gctccgcccccctgacgagcatcacaaaaatcgacgctcaagtcagaggtggcgaaacccga |
| | caggactataaagataccaggcgtttccccctggaagctccctcgtgcgctctcctgttccgacc |
| | ctgccgcttaccggatacctgtccgcctttctcccttcgggaagcgtggcgctttctcatagctca |
| | cgctgtaggtatctcagttcggtgtaggtcgttcgctccaagctgggctgtgtgcacgaaccccc |
| | cgttcagcccgaccgctgcgccttatccggtaactatcgtcttgagtccaacccggtaagacac |
| | gacttatcgccactggcagcagccactggtaacaggattagcagagcgaggtatgtaggcggt |
| | gctacagagttcttgaagtggtggcctaactacggctacactagaagaacagtatttggtatctg |
| | cgctctgctgaagccagttaccttcggaaaaagagttggtagctcttgatccggcaaacaaacc |
| | accgctggtagcggtggtttttttgtttgcaagcagcagattacgcgcagaaaaaaaggatctca |
| | agaagatcctttgatcttttctacggggtctgacgctcagtggaacgaaaactcacgttaaggga |
| | ttttggtcatgagattatcaaaaaggatcttcacctagatccttttaaattaaaaatgaagttttaaat |
| | caatctaaagtatatatgagtaaacttggtctgacagttaccaatgcttaatcagtgaggcacctat |
| | ctcagcgatctgtctatttcgttcatccatagttgcctgactccccgtcgtgtagataactacgatac |
| | gggagggcttaccatctggccccagtgctgcaatgataccgcgagaaccacgctcaccggct |
| | ccagatttatcagcaataaaccagccagccggaagggccgagcgcagaagtggtcctgcaac |
| | tttatccgcctccatccagtctattaattgttgccgggaagctagagtaagtagttcgccagttaat |
| | agtttgcgcaacgttgttgccattgctacaggcatcgtggtgtcacgctcgtcgtttggtatggctt |
| | cattcagctccggttcccaacgatcaaggcgagttacatgatcccccatgttgtgcaaaaaagc |
| | ggttagctccttcggtcctccgatcgttgtcagaagtaagttggccgcagtgttatcactcatggtt |
| | atggcagcactgcataattctcttactgtcatgccatccgtaagatgcttttctgtgactggtgagt |
| | actcaaccaagtcattctgagaatagtgtatgcggcgaccgagttgctcttgcccggcgtcaata |
| | cgggataataccgcgccacatagcagaactttaaaagtgctcatcattggaaaacgttcttcggg |
| | gcgaaaactctcaaggatcttaccgctgttgagatccagttcgatgtaacccactcgtgcaccca |
| | actgatcttcagcatcttttactttcaccagcgtttctgggtgagcaaaaacaggaaggcaaaatg |
| | ccgcaaaaaagggaataagggcgacacggaaatgttgaatactcatactcttcctttttcaatatt |
| | attgaagcatttatcagggttattgtctcatgagcggatacatatttgaatgtatttagaaaaataaa |
| | caaataggggttccgcgcacatttccccgaaaagtgccacctgacgtctaagaaaccattattat |
| | catgacattaacctataaaaataggcgtatcacgaggcccttttgtc (SEQ ID NO: 98) |
| pTarget(CyCAST) | cggggcggagatttcctggaagatgccaggaagatacttaacagggaagtgagagggccgc |
| | ggcaaagccgtttttccataggctccgcccccctgacaagcatcacgaaatctgacgctcaaat |
| | cagtggtggcgaaacccgacaggactataaagataccaggcgtttccccctggcggctccctc |
| | gtgcgctctcctgttcctgcctttcggtttaccggtgtcattccgctgttatggccgcgtttgtctcat |
| | tccacgcctgacactcagttccgggtaggcagttcgctccaagctggactgtatgcacgaaccc |
| | cccgttcagtccgaccgctgcgccttatccggtaactatcgtcttgagtccaacccggaaagac |
| | atgcaaaagcaccactggcagcagccactggtaattgatttagaggagttagtcttgaagtcatg |
| | cgccggttaaggctaaactgaaaggacaagttttggtgactgcgctcctccaagccagttacct |
| | cggttcaaagagttggtagctcagagaaccttcgaaaaaccgccctgcaaggcggttttttcgtt |
| | ttcagagcaagagattacgcgcagaccaaaacgatctcaagaagatcatcttattaatatacgta |
| | gtatctacgatacgtagtatctacgatacgtagnnnnnngagaagtcatttaataaggccactgt |
| | taaacgtagatatatctacgcgtagatatatctacgatacgtagcggccgacgcgctgggctac |
| | gtctttaatgggcagcgcccacatacgcagcgatttccaacagcttgaggcggaagctttaatg |
| | cggtagtttatcacagttaaattgctaacgcagtcaggcaccgtgtatgaaatctaacaatgcgct |
| | catcgtcatcctcggcaccgtcaccctggatgctgtaggcataggcttggttatgccggtactgc |
| | cgggcctcttgcgggatatcgtccattccgacagcatcgccagtcactatggcgtgctgctagc |
| | gctatatgcgttgatgcaatttctatgcgcacccgttctcggagcactgtccgaccgctttggccg |
| | ccgcccagtcctgctcgcttcgctacttggagccactatcgactacgcgatcatggcgaccaca |
| | cccgtcctgtggatcctctacgccggacgcatcgtggccggcatcaccggcgccacaggtgc |
| | ggttgctggcgcctatatcgccgacatcaccgatggggaagatcgggcctgccaccataccca |
| | cgccgaaacaagcgccctgcaccattatgttccggatctgcatcgcaggatgctgctggctacc |
| | ctgtggaacacctacatctgtattaacgaagcgctaaccgtttttatcaggctctgggaggcaga |
| | ataaatgatcatatcgtcaattattacctccacggggagagcctgagcaaactggcctcaggcat |
| | ttgagaagcacacggtcacactgcttccggtagtcaataaaccggtaaaccagcaatagacata |
| | agcggctatttaacgaccctgccctgaaccgacgaccgggtcgaatttgctttcgaatttctgcc |
| | attcatccgcttattatcacttattcaggcgtagcaaccaggcgtttaagggcaccaataactgcc |
| | ttaaaaaaattacgccccgccctgccactcatcgcagtactgttgtaattcattaagcattctgccg |
| | acatggaagccatcacaaacggcatgatgaacctgaatcgccagcggcatcagcaccttgtcg |
| | ccttgcgtataatatttgcccatggtgaaaacgggggcgaagaagttgtccatattggccacgttt |
| | aaatcaaaactggtgaaactcacccagggattggctgagacgaaaaacatattctcaataaacc |
| | ctttagggaaataggccaggttttcaccgtaacacgccacatcttgcgaatatatgtgtagaaact |
| | gccggaaatcgtcgtggtattcactccagagcgatgaaaacgtttcagtttgctcatggaaaacg |
| | gtgtaacaagggtgaacactatcccatatcaccagctcaccgtctttcattgccatacggaattcc |
| | ggatgagcattcatcaggcgggcaagaatgtgaataaaggccggataaaacttgtgcttattttt |
| | ctttacggtctttaaaaaggccgtaatatccagctgaacggtctggttataggtacattgagcaac |
| | tgactgaaatgcctcaaaatgttctttacgatgccattgggatatatcaacggtggtatatccagtg |
| | atttttttctccattttagcttccttagctcctgaaaatctcgataactcaaaaaatacgcccggcgg |
| | atgtggtggaattggtagacacgcacgtttgaggggcgtgtggcgtaagccttgcgagttcgat |
| | tctcgccatccgcagtagtgatcttatttcattatggtgaaagttggaacctcttacgtgccgatca |
| | acgtctcattttcgccaaaagttggcccagggcttcccggtatcaacagggacaccaggatttat |
| | ttattctgcgaagtgatcttccgtcacaggtatttattcggcgcaaagtgcgtcgggtgatgctgc |
| | caacttactgatttagtgtatgatggtgtttttgaggtgctccagtggcttctgtttctatcagctgtc |
| | cctcctgttcagctactgacggggtggtgcgtaacggcaaaagcaccgccggacatcagcgct |
| | agcggagtgtatactggcttactatgttggcactgatgagggtgtcagtgaagtgcttcatgtgg |
| | caggagaaaaaaggctgcaccggtgcgtcagcagaatatgtgatacaggatatattccgcttcc |
| | tcgctcactgactcgctacgctcggtcgttcgactgcggcgagcggaaatggcttacgaa |
| | (SEQ ID NO: 99) |
| pHelper(AjTn6022) | ggggaattgtgagcggataacaattcccctgtagaaataattttgtttaactttaataaggagatat |
| | acatgaaaagaatcagtgatgaatattccccagttaggaaatcccaaactgtatatgggagtattt |
| | cagtaaattatgcttttagaggggaaaagacgatttggtgtgaatctactcttgagcgtgattttttg |
| | attaggcaagaattcaatagcaatgtcatagatatcgtaagccagcctattaccatcccatatata |
| | acagagttgggtaatgaatccacttatactcctgatttcctcgtacagttttccagtgcaaattgtgg |
| | tgattttgaggaaattccctgtccactgttaatagaaataaaacctagaaagaaacttatagaagat |
| | tgggataaattaaagccaaaatttaaggcggctcatacttttgctaaagaaaaaggttggaaattc |
| | aagattataaatgaatcaaggttatatgatcagtattgggaaaatattaattttttgaaaagatttaga |
| | cgttctcatgtggatgtctatgatgaacaaactttaattgagactttagatagtcttggtcaatcgac |
| | tattaatcaattaccggcacatatttttaaggataaaactaatattcttcgaggtattcatcaagtatg |
| | ggcattggtcgcaaaacgaaaaatcatgtgtgatttatattgccccttaacagcagagacggtga |
| | tttggacaaatcaagatgatgtgcttacaaggagatttttaaatggttcagactgatactgtaagta |
| | ttgaccgaagtcggatcatactcaaaccaaatgtaattgttaagtatgaagggcaaccgtataag |
| | attgctaatgttttgaatgccaacgatatcgtgatttctagtttagattctgtgcgttgtttacaagta |
| | agtgcagattctctacaagtatttgaagctgaaaataaccaaactaaagatataaataaaggtgatt |
| | gggatattacgcatgaagcgtggaaaatagctttacatcgttatgaaatcatcaagccgttgatta |
| | agtattcaacaaccgaattagtcgagcaacgagcaagtgaatatggcattaaccgttccacctta |
| | tggaaatggttaaaagattttagagaaaataattctatcttagtgttagttcctaaaaagagaggtt |
| | ggacgaccaagaagtctaggttgtctcctcaaattatgaatattattaaccaaggtattcaagagg |
| | aatatctcaatgccagaaagcctagtattgcaaaaactatagattttattaaagcagaatgttctcg |
| | ccttaaactagaagcaccccatgacaattcaattcgtagaaaaatagaagcattaaacgattatat |
| | ggtgattaaggctcgtcatggttctaaagcagcacaagataagtatgcatctgctgctggtgagt |
| | ttcctaatgctgattatcccttggcatatgtacaggtagaccacactccactagatattgaaattgtt |
| | gatgatgaatttagagaggcgattggacgaccatttctaacattagctattgatataaatactcgaa |
| | tggtggtcggttattacttgtccttagaagcccccagtgcaacatctgttgccatgtgtattgcttcc |
| | agtattttgtcaaaaaaacggaagctaattgaattggatatagatgctgattggcaagttgaaggt |
| | cttatggattctatacatacagataatggtcctgactttagaactaaccatatcagtcgagcttgctt |
| | aaaatatgggattcattgggagtttcgtccgattggtggggcaaggtttggtggacatatcgaac |
| | gtttgattggtgttgttaacattgaaatgcacatcttggatggtacaaccttctcgaatatccaagaa |
| | cgtggaacctataattcagcaggtcatgcctgtatgacactaaaagaactagaatattatattgttt |
| | attggatcactaaagtttatcaccaaaagaaacactccaaactcggtatgtcacctttacagaagt |
| | ggcaagatggtgtttggggaacaaaaactagtgttggaacaggtttaaaagagagagtttcgga |
| | tgaagatacattatttattgatttccttcccgaatttgaatcaacaattcaacgtgtaggtgtacaga |
| | aagacaatctattttattttgctgattgtttacgacaatgggttaattgtgttgatcccgaagatgtaa |
| | ataaaaagagaaaaaagaaatttttgtttaaacgtgatccaagggatatttctcttatttggttttatg |
| | aacctctgtccaatacatattttaaagtgcctactgctaagcgagaaataccaccaattagcttattt |
| | gaatacaaacaagtgcagaaatatcttcgtggcgagcaacatgataatcaaaatcaggatgaga |
| | tttatcaggcaattcttcatttacgagagcaattgaaacaatcccgtagtctgactcggaagcaac |
| | ggagagtaaatcaacgtaaaaaggaaaatgcaaaagcaacacatcatttatctgtaaaaaatga |
| | acctgaaaaaccatctattgtcgaaaacagtgttcagccgactaatgatttatggagtattccattg |
| | acggcatttgaagatttaaggggataataatgcaaaaatatgaacatcttgcagatgatgtacttg |
| | aagtcatgtctctaagtgatagtgatcgtatcaatattttattttctgatcgttggattggttataaaa |
| | aagcggtcactattttggatactttaactgatctgttgaatcgccctaggaagcttcgcccagaatgt |
| | ttattgattgtgggtgattctaatatgggtaaaacgaccattatccacgaattcacaaagcagcatt |
| | acaccaaaacagtgaatgacatagatatggagctgctgtctgtaaaaaaacctgttttaaccatat |
| | tagcccctgctaaagcgaatgtaaaagaattctatatcaatatattgaatcatttctttgtcccatttc |
| | gtcctactgatcctgaagcaaagttacggaatcaagctgtccatctaatgcggaagtatgaaaca |
| | aaaatgttgattattgatgaaatccataattgtttaacaggttctaccaaacttctttccgaagtgatg |
| | aataccttaaagaatctaagtaatgatctaagtctgaatatcgttggcgtcggtactcgtgaagca |
| | gttacgattctacataccgatgctcaatatgcaagccgttttgatgtagcaatgttgcctaaatggg |
| | aactgaatcaggattttctaagactattattaagttatgtacgattattgcctttaaagaaacgttctg |
| | atctagcatctcaaagacttgccacccttatatttgaaatttcaggtgggaattttggtgacttaaac |
| | agattgttgattgagtgtgctaaagaggcaatcgtaaaaggtaatgaagaaattaccgaagaaat |
| | tgttaataaatttaaatggcttaaaccaaccgagggtatgaggaatattagaaatataaatcttaac |
| | atggtttaggtattctacttttcataggaaaatagccatgcaccaagtagaacaacgttggtatatt |
| | cgtacccctattcaagagggtgaagtattttcctcttggctcatacgttctgctttggatttaggctg |
| | ctcaccattaactttagtgaatcttttgtgggaaaaatggagaggattaacgatagatattgatcgt |
| | gggttaaatgaagaaaaattagacatattgttaaatcattgttttgtggaacagcatgaagttcaga |
| | aaactatgttaagcagttatttccctgctttgattccaaaaaattataacaaaaatcagcgtattcctt |
| | ggatattggctctaggtattcgaaatcgttcaaatgtgtcaggtcgtcaaatttgtacgcaatgtct |
| | aagttctacccatacaccaccatatttgcgtatcaagtggcgtatggggtggcatagtggctgtg |
| | atattcataaaaccatattaatagatcattgtcctcaatgcagaatcacattacaacctataaaaatt |
| | gatatagagcatggatcattagcaatttgtacttcatgctatgctgatttaagtacactcattcaacg |
| | tccagtagaaacggatgcattaagatttcagcatcaagctgatcaggtcttagagtcacaatttgg |
| | gcgatataaccaaaaggatatttcatcaatagagtggtttgagattgctcgtatatggttaagtttta |
| | ttcgatcccctttaaatatgaagagtcctaaccttttgcaaatgtttgaaagtcttaatattgatttaa |
| | atatagattatcctattacaccagtagcctttgaatttctgtctgttcaggagcgtgaaaaattattat |
| | caattttaaataaaatcatggcattaccttgtgatttatttgttgatcgctctttagaatatcatgtaa |
| | gtcattcttatttttgggataaaagaaaaaaactgcctacgcagcttcaactaatgaaagataaaatgt |
| | tgaagccaattaagattattttgccgaaagataacgccatagccatgagtaaacccaaatctaaaa |
| | aaagtgtacaacggaagtggttaaggctattgagaaagctacagaaaggggggaacaggtga |
| | ctttagctcaattttcccataaagaaggctgtagtggttgtgacagggctgatatagataaattcaa |
| | aaaaagatataagggcgataagtattgttcaacttgttatgcccgaatttttaaaaaaagagcttgt |
| | ccaaaatgtggtgaatatgccagattgccaaaaaatgatgatcaagcaatttgtaatgaatgcatc |
| | aaaaaacagccttgtattcgctgtaatcaaactaagaagccgatagggacactcaccgaatatg |
| | gtgtggtttgtaatagctgttctgtttattttcgcccaattgagcgttgtgagaggtgtgatacacca |
| | tcacaaaaattgacacatatttcacgatttggtgatgatttaagggtatgtcccaaatgctctactag |
| | agattatgaaacatgtccctcttgtcataaatatcgcttattagagcaagatgatacaggagctaa |
| | gatttgtaaaaaatgtaggaataacgccaaaaagaaatgcaagcaatgtgatgtcttgataccag |
| | caggctgtcctgatctatgtaacaactgttactggcataagaatctatgggagaaagctagacga |
| | aatattaaagcattccaatcccaacacttacaggctcaatatgagcaatatttaatttggttagaag |
| | atgaggttggtgcaaataaagcagcactgtatatcaataaacatacccatttttttatcaaaactga |
| | agaattatggcttgatgcaattcctacagcagaacagttattggctgttttaagaaccagtggactt |
| | agaaaatttgagttggttgcatcttggctagatgaagcccatcacattaaggttgcattagaggat |
| | aaagacttttgctctcaacaggatcagatagaaaaactgatttcaagccttcctcacccctctact |
| | gcttatgatgtggttatatcttataaagatgagcttgatataaaaatgaaagatggaaagaccagt |
| | atccggtccataaaactggcaataaagcctgcggttgctcttatgcactatgtttgtgcatcggga |
| | gctacattacctaatttaaaccatataaaagcctatcttatcgacttttctggtcaggctgctgcact |
| | aacagggttcattaattttttgaataaaaattttgatacatctatagactatttggcttttaaaaaatc |
| | aaaaaatttcaatgaaaaacgaaaaaataaagtagaaaaagaaattgtccaatgggtggataagc |
| | ctttggagaataaggaagatgtattaaattgggtgaagaatgggctaagatattttcataatgtatc |
| | ttatgtggagtctctaaaagtcaaatttgagatgattacagaagctgatgatggatatgaaattttat |
| | tgcagaatcattcttattggttgccaaaaaacacaggagatttaaaaagatagtttttttgtgattata |
| | ctgtgctatttcaagctaatttattgtacaagttggtttgtttatctaattcttctaaaagaactgtcc |
| | ataacaatatggggcaggctgctgccaccgctgagcaataactagcataaccccttggggcctct |
| | aaacgggtcttgaggggttttttgctgaaacctcaggcatttgagaagcacacggtcacactgct |
| | tccggtagtcaataaaccggtaaaccagcaatagacataagcggctatttaacgaccctgccct |
| | gaaccgacgaccgggtcatcgtggccggatcttgcggcccctcggcttgaacgaattgttaga |
| | cattatttgccgactaccttggtgatctcgcctttcacgtagtggacaaattcttccaactgatctgc |
| | gcgcgaggccaagcgatcttcttcttgtccaagataagcctgtctagcttcaagtatgacgggct |
| | gatactgggccggcaggcgctccattgcccagtcggcagcgacatccttcggcgcgattttgc |
| | cggttactgcgctgtaccaaatgcgggacaacgtaagcactacatttcgctcatcgccagccca |
| | gtcgggcggcgagttccatagcgttaaggtttcatttagcgcctcaaatagatcctgttcaggaa |
| | ccggatcaaagagttcctccgccgctggacctaccaaggcaacgctatgttctcttgcttttgtca |
| | gcaagatagccagatcaatgtcgatcgtggctggctcgaagatacctgcaagaatgtcattgcg |
| | ctgccattctccaaattgcagttcgcgcttagctggataacgccacggaatgatgtcgtcgtgca |
| | caacaatggtgacttctacagcgcggagaatctcgctctctccaggggaagccgaagtttccaa |
| | aaggtcgttgatcaaagctcgccgcgttgtttcatcaagccttacggtcaccgtaaccagcaaat |
| | caatatcactgtgtggcttcaggccgccatccactgcggagccgtacaaatgtacggccagca |
| | acgtcggttcgagatggcgctcgatgacgccaactacctctgatagttgagtcgatacttcggc |
| | gatcaccgcttccctcatactcttcctttttcaatattattgaagcatttatcagggttattgtctcat |
| | gagcggatacatatttgaatgtatttagaaaaataaacaaatagctagctcactcggtcgctacgctc |
| | cgggcgtgagactgcggcgggcgctgcggacacatacaaagttacccacagattccgtggat |
| | aagcaggggactaacatgtgaggcaaaacagcagggccgcgccggtggcgtttttccatagg |
| | ctccgccctcctgccagagttcacataaacagacgcttttccggtgcatctgtgggagccgtga |
| | ggctcaaccatgaatctgacagtacgggcgaaacccgacaggacttaaagatccccaccgttt |
| | ccggcgggtcgctccctcttgcgctctcctgttccgaccctgccgtttaccggatacctgttccgc |
| | ctttctcccttacgggaagtgtggcgctttctcatagctcacacactggtatctcggctcggtgta |
| | ggtcgttcgctccaagctgggctgtaagcaagaactccccgttcagcccgactgctgcgcctta |
| | tccggtaactgttcacttgagtccaacccggaaaagcacggtaaaacgccactggcagcagcc |
| | attggtaactgggagttcgcagaggatttgtttagctaaacacgcggttgctcttgaagtgtgcgc |
| | caaagtccggctacactggaaggacagatttggttgctgtgctctgcgaaagccagttaccacg |
| | gttaagcagttccccaactgacttaaccttcgatcaaaccacctccccaggtggttttttcgtttac |
| | agggcaaaagattacgcgcagaaaaaaaggatctcaagaagatcctttgatcttttctactgaac |
| | cgctctagatttcagtgcaatttatctcttcaaatgtagcacctgaagtcagccccatacgatataa |
| | gttgtaattctcatgttagtcatgccccgcgcccaccggaaggagctgactgggttgaaggctct |
| | caagggcatcggtcgagatcccggtgcctaatgagtgagctaacttacattaattgcgttgcgct |
| | cactgcccgctttccagtcgggaaacctgtcgtgccagctgcattaatgaatcggccaacgcgc |
| | ggggagaggcggtttgcgtattgggcgccagggtggtttttcttttcaccagtgagacgggcaa |
| | cagctgattgcccttcaccgcctggccctgagagagttgcagcaagcggtccacgctggtttgc |
| | cccagcaggcgaaaatcctgtttgatggtggttaacggcgggatataacatgagctgtcttcggt |
| | atcgtcgtatcccactaccgagatgtccgcaccaacgcgcagcccggactcggtaatggcgc |
| | gcattgcgcccagcgccatctgatcgttggcaaccagcatcgcagtgggaacgatgccctcatt |
| | cagcatttgcatggtttgttgaaaaccggacatggcactccagtcgccttcccgttccgctatcgg |
| | ctgaatttgattgcgagtgagatatttatgccagccagccagacgcagacgcgccgagacaga |
| | acttaatgggcccgctaacagcgcgatttgctggtgacccaatgcgaccagatgctccacgcc |
| | cagtcgcgtaccgtcttcatgggagaaaataatactgttgatgggtgtctggtcagagacatcaa |
| | gaaataacgccggaacattagtgcaggcagcttccacagcaatggcatcctggtcatccagcg |
| | gatagttaatgatcagcccactgacgcgttgcgcgagaagattgtgcaccgccgctttacaggc |
| | ttcgacgccgcttcgttctaccatcgacaccaccacgctggcacccagttgatcggcgcgagat |
| | ttaatcgccgcgacaatttgcgacggcgcgtgcagggccagactggaggtggcaacgccaat |
| | cagcaacgactgtttgcccgccagttgttgtgccacgcggttgggaatgtaattcagctccgcc |
| | atcgccgcttccactttttcccgcgttttcgcagaaacgtggctggcctggttcaccacgcggga |
| | aacggtctgataagagacaccggcatactctgcgacatcgtataacgttactggtttcacattcac |
| | caccctgaattgactctcttccgggcgctatcatgccataccgcgaaaggttttgcgccattcgat |
| | ggtgtccgggatctcgacgctctcccttatgcgactcctgcattaggaaattaatacgactcacta |
| | ta (SEQ ID NO: 100) |
| pHelper(AjTn6022)_ | Ggggaattgtgagcggataacaattcccctgtagaaataattttgtttaactttaataaggagatat |
| ΔTnsA | acatggttcagactgatactgtaagtattgaccgaagtcggatcatactcaaaccaaatgtaattg |
| | ttaagtatgaagggcaaccgtataagattgctaatgttttgaatgccaacgatatcgtgatttctag |
| | tttagattctgtgcgttgtttacaagtaagtgcagattctctacaagtatttgaagctgaaaataacc |
| | aaactaaagatataaataaaggtgattgggatattacgcatgaagcgtggaaaatagctttacat |
| | cgttatgaaatcatcaagccgttgattaagtattcaacaaccgaattagtcgagcaacgagcaag |
| | tgaatatggcattaaccgttccaccttatggaaatggttaaaagattttagagaaaataattctatct |
| | tagtgttagttcctaaaaagagaggttggacgaccaagaagtctaggttgtctcctcaaattatga |
| | atattattaaccaaggtattcaagaggaatatctcaatgccagaaagcctagtattgcaaaaacta |
| | tagattttattaaagcagaatgttctcgccttaaactagaagcaccccatgacaattcaattcgtag |
| | aaaaatagaagcattaaacgattatatggtgattaaggctcgtcatggttctaaagcagcacaag |
| | ataagtatgcatctgctgctggtgagtttcctaatgctgattatcccttggcatatgtacaggtaga |
| | ccacactccactagatattgaaattgttgatgatgaatttagagaggcgattggacgaccatttcta |
| | acattagctattgatataaatactcgaatggtggtcggttattacttgtccttagaagcccccagtg |
| | caacatctgttgccatgtgtattgcttccagtattttgtcaaaaaaacggaagctaattgaattggat |
| | atagatgctgattggcaagttgaaggtcttatggattctatacatacagataatggtcctgacttta |
| | gaactaaccatatcagtcgagcttgcttaaaatatgggattcattgggagtttcgtccgattggtg |
| | gggcaaggtttggtggacatatcgaacgtttgattggtgttgttaacattgaaatgcacatcttgg |
| | atggtacaaccttctcgaatatccaagaacgtggaacctataattcagcaggtcatgcctgtatga |
| | cactaaaagaactagaatattatattgtttattggatcactaaagtttatcaccaaaagaaacactc |
| | caaactcggtatgtcacctttacagaagtggcaagatggtgtttggggaacaaaaactagtgttg |
| | gaacaggtttaaaagagagagtttcggatgaagatacattatttattgatttccttcccgaatttgaa |
| | tcaacaattcaacgtgtaggtgtacagaaagacaatctattttattttgctgattgtttacgacaatg |
| | ggttaattgtgttgatcccgaagatgtaaataaaaagagaaaaaagaaatttttgtttaaacgtgat |
| | ccaagggatatttctcttatttggttttatgaacctctgtccaatacatattttaaagtgcctactgct |
| | aagcgagaaataccaccaattagcttatttgaatacaaacaagtgcagaaatatcttcgtggcgag |
| | caacatgataatcaaaatcaggatgagatttatcaggcaattcttcatttacgagagcaattgaaa |
| | caatcccgtagtctgactcggaagcaacggagagtaaatcaacgtaaaaaggaaaatgcaaa |
| | agcaacacatcatttatctgtaaaaaatgaacctgaaaaaccatctattgtcgaaaacagtgttca |
| | gccgactaatgatttatggagtattccattgacggcatttgaagatttaaggggataataatgcaa |
| | aaatatgaacatcttgcagatgatgtacttgaagtcatgtctctaagtgatagtgatcgtatcaatat |
| | tttattttctgatcgttggattggttataaaaaagcggtcactattttggatactttaactgatctgtt |
| | gaatcgccctaggaagcttcgcccagaatgtttattgattgtgggtgattctaatatgggtaaaacga |
| | ccattatccacgaattcacaaagcagcattacaccaaaacagtgaatgacatagatatggagct |
| | gctgtctgtaaaaaaacctgttttaaccatattagcccctgctaaagcgaatgtaaaagaattctat |
| | atcaatatattgaatcatttctttgtcccatttcgtcctactgatcctgaagcaaagttacggaatcaa |
| | gctgtccatctaatgcggaagtatgaaacaaaaatgttgattattgatgaaatccataattgtttaac |
| | aggttctaccaaacttctttccgaagtgatgaataccttaaagaatctaagtaatgatctaagtctg |
| | aatatcgttggcgtcggtactcgtgaagcagttacgattctacataccgatgctcaatatgcaagc |
| | cgttttgatgtagcaatgttgcctaaatgggaactgaatcaggattttctaagactattattaagttat |
| | gtacgattattgcctttaaagaaacgttctgatctagcatctcaaagacttgccacccttatatttga |
| | aatttcaggtgggaattttggtgacttaaacagattgttgattgagtgtgctaaagaggcaatcgta |
| | aaaggtaatgaagaaattaccgaagaaattgttaataaatttaaatggcttaaaccaaccgaggg |
| | tatgaggaatattagaaatataaatcttaacatggtttaggtattctacttttcataggaaaatagcc |
| | atgcaccaagtagaacaacgttggtatattcgtacccctattcaagagggtgaagtattttcctctt |
| | ggctcatacgttctgctttggatttaggctgctcaccattaactttagtgaatcttttgtgggaaaaat |
| | ggagaggattaacgatagatattgatcgtgggttaaatgaagaaaaattagacatattgttaaatc |
| | attgttttgtggaacagcatgaagttcagaaaactatgttaagcagttatttccctgctttgattcca |
| | aaaaattataacaaaaatcagcgtattccttggatattggctctaggtattcgaaatcgttcaaatgt |
| | gtcaggtcgtcaaatttgtacgcaatgtctaagttctacccatacaccaccatatttgcgtatcaag |
| | tggcgtatggggtggcatagtggctgtgatattcataaaaccatattaatagatcattgtcctcaat |
| | gcagaatcacattacaacctataaaaattgatatagagcatggatcattagcaatttgtacttcatg |
| | ctatgctgatttaagtacactcattcaacgtccagtagaaacggatgcattaagatttcagcatcaa |
| | gctgatcaggtcttagagtcacaatttgggcgatataaccaaaaggatatttcatcaatagagtgg |
| | tttgagattgctcgtatatggttaagttttattcgatcccctttaaatatgaagagtcctaaccttttg |
| | caaatgtttgaaagtcttaatattgatttaaatatagattatcctattacaccagtagcctttgaattt |
| | ctgtctgttcaggagcgtgaaaaattattatcaattttaaataaaatcatggcattaccttgtgattta |
| | tttgttgatcgctctttagaatatcatgtaagtcattcttatttttgggataaaagaaaaaaactgcct |
| | acgcagcttcaactaatgaaagataaaatgttgaagccaattaagattattttgccgaaagataacgcc |
| | atagccatgagtaaacccaaatctaaaaaaagtgtacaacggaagtggttaaggctattgagaa |
| | agctacagaaaggggggaacaggtgactttagctcaattttcccataaagaaggctgtagtggt |
| | tgtgacagggctgatatagataaattcaaaaaaagatataagggcgataagtattgttcaacttgt |
| | tatgcccgaatttttaaaaaaagagcttgtccaaaatgtggtgaatatgccagattgccaaaaaat |
| | gatgatcaagcaatttgtaatgaatgcatcaaaaaacagccttgtattcgctgtaatcaaactaag |
| | aagccgatagggacactcaccgaatatggtgtggtttgtaatagctgttctgtttattttcgcccaa |
| | ttgagcgttgtgagaggtgtgatacaccatcacaaaaattgacacatatttcacgatttggtgatg |
| | atttaagggtatgtcccaaatgctctactagagattatgaaacatgtccctcttgtcataaatatcgc |
| | ttattagagcaagatgatacaggagctaagatttgtaaaaaatgtaggaataacgccaaaaaga |
| | aatgcaagcaatgtgatgtcttgataccagcaggctgtcctgatctatgtaacaactgttactggc |
| | ataagaatctatgggagaaagctagacgaaatattaaagcattccaatcccaacacttacaggct |
| | caatatgagcaatatttaatttggttagaagatgaggttggtgcaaataaagcagcactgtatatc |
| | aataaacatacccatttttttatcaaaactgaagaattatggcttgatgcaattcctacagcagaac |
| | agttattggctgttttaagaaccagtggacttagaaaatttgagttggttgcatcttggctagatga |
| | agcccatcacattaaggttgcattagaggataaagacttttgctctcaacaggatcagatagaaa |
| | aactgatttcaagccttcctcacccctctactgcttatgatgtggttatatcttataaagatgagcttg |
| | atataaaaatgaaagatggaaagaccagtatccggtccataaaactggcaataaagcctgcggt |
| | tgctcttatgcactatgtttgtgcatcgggagctacattacctaatttaaaccatataaaagcctatct |
| | tatcgacttttctggtcaggctgctgcactaacagggttcattaattttttgaataaaaattttgatac |
| | atctatagactatttggcttttaaaaaatcaaaaaatttcaatgaaaaacgaaaaaataaagtagaa |
| | aaagaaattgtccaatgggtggataagcctttggagaataaggaagatgtattaaattgggtgaa |
| | gaatgggctaagatattttcataatgtatcttatgtggagtctctaaaagtcaaatttgagatgatta |
| | cagaagctgatgatggatatgaaattttattgcagaatcattcttattggttgccaaaaaacacagg |
| | agatttaaaaagatagtttttttgtgattatactgtgctatttcaagctaatttattgtacaagttggt |
| | ttgtttatctaattcttctaaaagaactgtccataacaatatggggcaggctgctgccaccgctgagc |
| | aataactagcataaccccttggggcctctaaacgggtcttgaggggttttttgctgaaacctcag |
| | gcatttgagaagcacacggtcacactgcttccggtagtcaataaaccggtaaaccagcaataga |
| | cataagcggctatttaacgaccctgccctgaaccgacgaccgggtcatcgtggccggatcttgc |
| | ggcccctcggcttgaacgaattgttagacattatttgccgactaccttggtgatctcgcctttcacg |
| | tagtggacaaattcttccaactgatctgcgcgcgaggccaagcgatcttcttcttgtccaagataa |
| | gcctgtctagcttcaagtatgacgggctgatactgggccggcaggcgctccattgcccagtcg |
| | gcagcgacatccttcggcgcgattttgccggttactgcgctgtaccaaatgcgggacaacgtaa |
| | gcactacatttcgctcatcgccagcccagtcgggcggcgagttccatagcgttaaggtttcattta |
| | gcgcctcaaatagatcctgttcaggaaccggatcaaagagttcctccgccgctggacctaccaa |
| | ggcaacgctatgttctcttgcttttgtcagcaagatagccagatcaatgtcgatcgtggctggctc |
| | gaagatacctgcaagaatgtcattgcgctgccattctccaaattgcagttcgcgcttagctggata |
| | acgccacggaatgatgtcgtcgtgcacaacaatggtgacttctacagcgcggagaatctcgctc |
| | tctccaggggaagccgaagtttccaaaaggtcgttgatcaaagctcgccgcgttgtttcatcaag |
| | ccttacggtcaccgtaaccagcaaatcaatatcactgtgtggcttcaggccgccatccactgcg |
| | gagccgtacaaatgtacggccagcaacgtcggttcgagatggcgctcgatgacgccaactac |
| | ctctgatagttgagtcgatacttcggcgatcaccgcttccctcatactcttcctttttcaatattattg |
| | aagcatttatcagggttattgtctcatgagcggatacatatttgaatgtatttagaaaaataaacaaat |
| | agctagctcactcggtcgctacgctccgggcgtgagactgcggcgggcgctgcggacacata |
| | caaagttacccacagattccgtggataagcaggggactaacatgtgaggcaaaacagcaggg |
| | ccgcgccggtggcgtttttccataggctccgccctcctgccagagttcacataaacagacgcttt |
| | tccggtgcatctgtgggagccgtgaggctcaaccatgaatctgacagtacgggcgaaacccg |
| | acaggacttaaagatccccaccgtttccggcgggtcgctccctcttgcgctctcctgttccgacc |
| | ctgccgtttaccggatacctgttccgcctttctcccttacgggaagtgtggcgctttctcatagctc |
| | acacactggtatctcggctcggtgtaggtcgttcgctccaagctgggctgtaagcaagaactcc |
| | ccgttcagcccgactgctgcgccttatccggtaactgttcacttgagtccaacccggaaaagca |
| | cggtaaaacgccactggcagcagccattggtaactgggagttcgcagaggatttgtttagctaa |
| | acacgcggttgctcttgaagtgtgcgccaaagtccggctacactggaaggacagatttggttgc |
| | tgtgctctgcgaaagccagttaccacggttaagcagttccccaactgacttaaccttcgatcaaa |
| | ccacctccccaggtggttttttcgtttacagggcaaaagattacgcgcagaaaaaaaggatctca |
| | agaagatcctttgatcttttctactgaaccgctctagatttcagtgcaatttatctcttcaaatgtagc |
| | acctgaagtcagccccatacgatataagttgtaattctcatgttagtcatgccccgcgcccaccg |
| | gaaggagctgactgggttgaaggctctcaagggcatcggtcgagatcccggtgcctaatgagt |
| | gagctaacttacattaattgcgttgcgctcactgcccgctttccagtcgggaaacctgtcgtgcca |
| | gctgcattaatgaatcggccaacgcgcggggagaggcggtttgcgtattgggcgccagggtg |
| | gtttttcttttcaccagtgagacgggcaacagctgattgcccttcaccgcctggccctgagagag |
| | ttgcagcaagcggtccacgctggtttgccccagcaggcgaaaatcctgtttgatggtggttaac |
| | ggcgggatataacatgagctgtcttcggtatcgtcgtatcccactaccgagatgtccgcaccaa |
| | cgcgcagcccggactcggtaatggcgcgcattgcgcccagcgccatctgatcgttggcaacc |
| | agcatcgcagtgggaacgatgccctcattcagcatttgcatggtttgttgaaaaccggacatgg |
| | cactccagtcgccttcccgttccgctatcggctgaatttgattgcgagtgagatatttatgccagc |
| | cagccagacgcagacgcgccgagacagaacttaatgggcccgctaacagcgcgatttgctg |
| | gtgacccaatgcgaccagatgctccacgcccagtcgcgtaccgtcttcatgggagaaaataat |
| | actgttgatgggtgtctggtcagagacatcaagaaataacgccggaacattagtgcaggcagct |
| | tccacagcaatggcatcctggtcatccagcggatagttaatgatcagcccactgacgcgttgcg |
| | cgagaagattgtgcaccgccgctttacaggcttcgacgccgcttcgttctaccatcgacaccac |
| | cacgctggcacccagttgatcggcgcgagatttaatcgccgcgacaatttgcgacggcgcgtg |
| | cagggccagactggaggtggcaacgccaatcagcaacgactgtttgcccgccagttgttgtgc |
| | cacgcggttgggaatgtaattcagctccgccatcgccgcttccactttttcccgcgttttcgcaga |
| | aacgtggctggcctggttcaccacgcgggaaacggtctgataagagacaccggcatactctgc |
| | gacatcgtataacgttactggtttcacattcaccaccctgaattgactctcttccgggcgctatcat |
| | gccataccgcgaaaggttttgcgccattcgatggtgtccgggatctcgacgctctcccttatgcg |
| | actcctgcattaggaaattaatacgactcactata (SEQ ID NO: 101) |
| pHelper(AjTn6022)_ | ggggaattgtgagcggataacaattcccctgtagaaataattttgtttaactttaataaggagatat |
| ΔTnsB | acatgaaaagaatcagtgatgaatattccccagttaggaaatcccaaactgtatatgggagtattt |
| | cagtaaattatgcttttagaggggaaaagacgatttggtgtgaatctactcttgagcgtgattttttg |
| | attaggcaagaattcaatagcaatgtcatagatatcgtaagccagcctattaccatcccatatata |
| | acagagttgggtaatgaatccacttatactcctgatttcctcgtacagttttccagtgcaaattgtgg |
| | tgattttgaggaaattccctgtccactgttaatagaaataaaacctagaaagaaacttatagaagat |
| | tgggataaattaaagccaaaatttaaggcggctcatacttttgctaaagaaaaaggttggaaattc |
| | aagattataaatgaatcaaggttatatgatcagtattgggaaaatattaattttttgaaaagatttaga |
| | cgttctcatgtggatgtctatgatgaacaaactttaattgagactttagatagtcttggtcaatcgac |
| | tattaatcaattaccggcacatatttttaaggataaaactaatattcttcgaggtattcatcaagtatg |
| | ggcattggtcgcaaaacgaaaaatcatgtgtgatttatattgccccttaacagcagagacggtga |
| | tttggacaaatcaagatgatgtgcttacaaggagatttttaaatggttcagactgataatgcaaaaa |
| | tatgaacatcttgcagatgatgtacttgaagtcatgtctctaagtgatagtgatcgtatcaatatttta |
| | ttttctgatcgttggattggttataaaaaagcggtcactattttggatactttaactgatctgttgaat |
| | cgccctaggaagcttcgcccagaatgtttattgattgtgggtgattctaatatgggtaaaacgacca |
| | ttatccacgaattcacaaagcagcattacaccaaaacagtgaatgacatagatatggagctgctg |
| | tctgtaaaaaaacctgttttaaccatattagcccctgctaaagcgaatgtaaaagaattctatatca |
| | atatattgaatcatttctttgtcccatttcgtcctactgatcctgaagcaaagttacggaatcaagct |
| | gtccatctaatgcggaagtatgaaacaaaaatgttgattattgatgaaatccataattgtttaacag |
| | gttctaccaaacttctttccgaagtgatgaataccttaaagaatctaagtaatgatctaagtctgaat |
| | atcgttggcgtcggtactcgtgaagcagttacgattctacataccgatgctcaatatgcaagccgt |
| | tttgatgtagcaatgttgcctaaatgggaactgaatcaggattttctaagactattattaagttatgta |
| | cgattattgcctttaaagaaacgttctgatctagcatctcaaagacttgccacccttatatttgaaatt |
| | tcaggtgggaattttggtgacttaaacagattgttgattgagtgtgctaaagaggcaatcgtaaaa |
| | ggtaatgaagaaattaccgaagaaattgttaataaatttaaatggcttaaaccaaccgagggtat |
| | gaggaatattagaaatataaatcttaacatggtttaggtattctacttttcataggaaaatagccatg |
| | caccaagtagaacaacgttggtatattcgtacccctattcaagagggtgaagtattttcctcttggc |
| | tcatacgttctgctttggatttaggctgctcaccattaactttagtgaatcttttgtgggaaaaatgga |
| | gaggattaacgatagatattgatcgtgggttaaatgaagaaaaattagacatattgttaaatcattg |
| | ttttgtggaacagcatgaagttcagaaaactatgttaagcagttatttccctgctttgattccaaaaa |
| | attataacaaaaatcagcgtattccttggatattggctctaggtattcgaaatcgttcaaatgtgtca |
| | ggtcgtcaaatttgtacgcaatgtctaagttctacccatacaccaccatatttgcgtatcaagtggc |
| | gtatggggtggcatagtggctgtgatattcataaaaccatattaatagatcattgtcctcaatgcag |
| | aatcacattacaacctataaaaattgatatagagcatggatcattagcaatttgtacttcatgctatg |
| | ctgatttaagtacactcattcaacgtccagtagaaacggatgcattaagatttcagcatcaagctg |
| | atcaggtcttagagtcacaatttgggcgatataaccaaaaggatatttcatcaatagagtggtttg |
| | agattgctcgtatatggttaagttttattcgatcccctttaaatatgaagagtcctaaccttttgcaaa |
| | tgtttgaaagtcttaatattgatttaaatatagattatcctattacaccagtagcctttgaatttctgt |
| | ctgttcaggagcgtgaaaaattattatcaattttaaataaaatcatggcattaccttgtgatttatttg |
| | ttgatcgctctttagaatatcatgtaagtcattcttatttttgggataaaagaaaaaaactgcctacgc |
| | agcttcaactaatgaaagataaaatgttgaagccaattaagattattttgccgaaagataacgccata |
| | gccatgagtaaacccaaatctaaaaaaagtgtacaacggaagtggttaaggctattgagaaag |
| | ctacagaaaggggggaacaggtgactttagctcaattttcccataaagaaggctgtagtggttgt |
| | gacagggctgatatagataaattcaaaaaaagatataagggcgataagtattgttcaacttgttat |
| | gcccgaatttttaaaaaaagagcttgtccaaaatgtggtgaatatgccagattgccaaaaaatgat |
| | gatcaagcaatttgtaatgaatgcatcaaaaaacagccttgtattcgctgtaatcaaactaagaag |
| | ccgatagggacactcaccgaatatggtgtggtttgtaatagctgttctgtttattttcgcccaattga |
| | gcgttgtgagaggtgtgatacaccatcacaaaaattgacacatatttcacgatttggtgatgattta |
| | agggtatgtcccaaatgctctactagagattatgaaacatgtccctcttgtcataaatatcgcttatt |
| | agagcaagatgatacaggagctaagatttgtaaaaaatgtaggaataacgccaaaaagaaatg |
| | caagcaatgtgatgtcttgataccagcaggctgtcctgatctatgtaacaactgttactggcataa |
| | gaatctatgggagaaagctagacgaaatattaaagcattccaatcccaacacttacaggctcaat |
| | atgagcaatatttaatttggttagaagatgaggttggtgcaaataaagcagcactgtatatcaata |
| | aacatacccatttttttatcaaaactgaagaattatggcttgatgcaattcctacagcagaacagtta |
| | ttggctgttttaagaaccagtggacttagaaaatttgagttggttgcatcttggctagatgaagccc |
| | atcacattaaggttgcattagaggataaagacttttgctctcaacaggatcagatagaaaaactga |
| | tttcaagccttcctcacccctctactgcttatgatgtggttatatcttataaagatgagcttgatataa |
| | aaatgaaagatggaaagaccagtatccggtccataaaactggcaataaagcctgcggttgctct |
| | tatgcactatgtttgtgcatcgggagctacattacctaatttaaaccatataaaagcctatcttatcg |
| | acttttctggtcaggctgctgcactaacagggttcattaattttttgaataaaaattttgatacatcta |
| | tagactatttggcttttaaaaaatcaaaaaatttcaatgaaaaacgaaaaaataaagtagaaaaag |
| | aaattgtccaatgggtggataagcctttggagaataaggaagatgtattaaattgggtgaagaat |
| | gggctaagatattttcataatgtatcttatgtggagtctctaaaagtcaaatttgagatgattacaga |
| | agctgatgatggatatgaaattttattgcagaatcattcttattggttgccaaaaaacacaggagat |
| | ttaaaaagatagtttttttgtgattatactgtgctatttcaagctaatttattgtacaagttggtttgt |
| | ttatctaattcttctaaaagaactgtccataacaatatggggcaggctgctgccaccgctgagcaataa |
| | ctagcataaccccttggggcctctaaacgggtcttgaggggttttttgctgaaacctcaggcattt |
| | gagaagcacacggtcacactgcttccggtagtcaataaaccggtaaaccagcaatagacataa |
| | gcggctatttaacgaccctgccctgaaccgacgaccgggtcatcgtggccggatcttgcggcc |
| | cctcggcttgaacgaattgttagacattatttgccgactaccttggtgatctcgcctttcacgtagt |
| | ggacaaattcttccaactgatctgcgcgcgaggccaagcgatcttcttcttgtccaagataagcc |
| | tgtctagcttcaagtatgacgggctgatactgggccggcaggcgctccattgcccagtcggca |
| | gcgacatccttcggcgcgattttgccggttactgcgctgtaccaaatgcgggacaacgtaagca |
| | ctacatttcgctcatcgccagcccagtcgggcggcgagttccatagcgttaaggtttcatttagc |
| | gcctcaaatagatcctgttcaggaaccggatcaaagagttcctccgccgctggacctaccaag |
| | gcaacgctatgttctcttgcttttgtcagcaagatagccagatcaatgtcgatcgtggctggctcg |
| | aagatacctgcaagaatgtcattgcgctgccattctccaaattgcagttcgcgcttagctggataa |
| | cgccacggaatgatgtcgtcgtgcacaacaatggtgacttctacagcgcggagaatctcgctct |
| | ctccaggggaagccgaagtttccaaaaggtcgttgatcaaagctcgccgcgttgtttcatcaag |
| | ccttacggtcaccgtaaccagcaaatcaatatcactgtgtggcttcaggccgccatccactgcg |
| | gagccgtacaaatgtacggccagcaacgtcggttcgagatggcgctcgatgacgccaactac |
| | ctctgatagttgagtcgatacttcggcgatcaccgcttccctcatactcttcctttttcaatattattg |
| | aagcatttatcagggttattgtctcatgagcggatacatatttgaatgtatttagaaaaataaacaaat |
| | agctagctcactcggtcgctacgctccgggcgtgagactgcggcgggcgctgcggacacata |
| | caaagttacccacagattccgtggataagcaggggactaacatgtgaggcaaaacagcaggg |
| | ccgcgccggtggcgtttttccataggctccgccctcctgccagagttcacataaacagacgcttt |
| | tccggtgcatctgtgggagccgtgaggctcaaccatgaatctgacagtacgggcgaaacccg |
| | acaggacttaaagatccccaccgtttccggcgggtcgctccctcttgcgctctcctgttccgacc |
| | ctgccgtttaccggatacctgttccgcctttctcccttacgggaagtgtggcgctttctcatagctc |
| | acacactggtatctcggctcggtgtaggtcgttcgctccaagctgggctgtaagcaagaactcc |
| | ccgttcagcccgactgctgcgccttatccggtaactgttcacttgagtccaacccggaaaagca |
| | cggtaaaacgccactggcagcagccattggtaactgggagttcgcagaggatttgtttagctaa |
| | acacgcggttgctcttgaagtgtgcgccaaagtccggctacactggaaggacagatttggttgc |
| | tgtgctctgcgaaagccagttaccacggttaagcagttccccaactgacttaaccttcgatcaaa |
| | ccacctccccaggtggttttttcgtttacagggcaaaagattacgcgcagaaaaaaaggatctca |
| | agaagatcctttgatcttttctactgaaccgctctagatttcagtgcaatttatctcttcaaatgtagc |
| | acctgaagtcagccccatacgatataagttgtaattctcatgttagtcatgccccgcgcccaccg |
| | gaaggagctgactgggttgaaggctctcaagggcatcggtcgagatcccggtgcctaatgagt |
| | gagctaacttacattaattgcgttgcgctcactgcccgctttccagtcgggaaacctgtcgtgcca |
| | gctgcattaatgaatcggccaacgcgcggggagaggcggtttgcgtattgggcgccagggtg |
| | gtttttttttcaccagtgagacgggcaacagctgattgcccttcaccgcctggccctgagagag |
| | ttgcagcaagcggtccacgctggtttgccccagcaggcgaaaatcctgtttgatggtggttaac |
| | ggcgggatataacatgagctgtcttcggtatcgtcgtatcccactaccgagatgtccgcaccaa |
| | cgcgcagcccggactcggtaatggcgcgcattgcgcccagcgccatctgatcgttggcaacc |
| | agcatcgcagtgggaacgatgccctcattcagcatttgcatggtttgttgaaaaccggacatgg |
| | cactccagtcgccttcccgttccgctatcggctgaatttgattgcgagtgagatatttatgccagc |
| | cagccagacgcagacgcgccgagacagaacttaatgggcccgctaacagcgcgatttgctg |
| | gtgacccaatgcgaccagatgctccacgcccagtcgcgtaccgtcttcatgggagaaaataat |
| | actgttgatgggtgtctggtcagagacatcaagaaataacgccggaacattagtgcaggcagct |
| | tccacagcaatggcatcctggtcatccagcggatagttaatgatcagcccactgacgcgttgcg |
| | cgagaagattgtgcaccgccgctttacaggcttcgacgccgcttcgttctaccatcgacaccac |
| | cacgctggcacccagttgatcggcgcgagatttaatcgccgcgacaatttgcgacggcgcgtg |
| | cagggccagactggaggtggcaacgccaatcagcaacgactgtttgcccgccagttgttgtgc |
| | cacgcggttgggaatgtaattcagctccgccatcgccgcttccactttttcccgcgttttcgcaga |
| | aacgtggctggcctggttcaccacgcgggaaacggtctgataagagacaccggcatactctgc |
| | gacatcgtataacgttactggtttcacattcaccaccctgaattgactctcttccgggcgctatcat |
| | gccataccgcgaaaggttttgcgccattcgatggtgtccgggatctcgacgctctcccttatgcg |
| | actcctgcattaggaaattaatacgactcactata (SEQ ID NO: 102) |
| pHelper(AjTn6022)_ | gggaattgtgagcggataacaattcccctgtagaaataattttgtttaactttaataaggagatat |
| ΔTnsC | acatgaaaagaatcagtgatgaatattccccagttaggaaatcccaaactgtatatgggagtattt |
| | cagtaaattatgcttttagaggggaaaagacgatttggtgtgaatctactcttgagcgtgattttttg |
| | attaggcaagaattcaatagcaatgtcatagatatcgtaagccagcctattaccatcccatatata |
| | acagagttgggtaatgaatccacttatactcctgatttcctcgtacagttttccagtgcaaattgtgg |
| | tgattttgaggaaattccctgtccactgttaatagaaataaaacctagaaagaaacttatagaagat |
| | tgggataaattaaagccaaaatttaaggcggctcatacttttgctaaagaaaaaggttggaaattc |
| | aagattataaatgaatcaaggttatatgatcagtattgggaaaatattaattttttgaaaagatttaga |
| | cgttctcatgtggatgtctatgatgaacaaactttaattgagactttagatagtcttggtcaatcgac |
| | tattaatcaattaccggcacatatttttaaggataaaactaatattcttcgaggtattcatcaagtatg |
| | ggcattggtcgcaaaacgaaaaatcatgtgtgatttatattgccccttaacagcagagacggtga |
| | tttggacaaatcaagatgatgtgcttacaaggagatttttaaatggttcagactgatactgtaagta |
| | ttgaccgaagtcggatcatactcaaaccaaatgtaattgttaagtatgaagggcaaccgtataag |
| | attgctaatgttttgaatgccaacgatatcgtgatttctagtttagattctgtgcgttgtttacaagta |
| | agtgcagattctctacaagtatttgaagctgaaaataaccaaactaaagatataaataaaggtgatt |
| | gggatattacgcatgaagcgtggaaaatagctttacatcgttatgaaatcatcaagccgttgatta |
| | agtattcaacaaccgaattagtcgagcaacgagcaagtgaatatggcattaaccgttccacctta |
| | tggaaatggttaaaagattttagagaaaataattctatcttagtgttagttcctaaaaagagaggtt |
| | ggacgaccaagaagtctaggttgtctcctcaaattatgaatattattaaccaaggtattcaagagg |
| | aatatctcaatgccagaaagcctagtattgcaaaaactatagattttattaaagcagaatgttctcg |
| | ccttaaactagaagcaccccatgacaattcaattcgtagaaaaatagaagcattaaacgattatat |
| | ggtgattaaggctcgtcatggttctaaagcagcacaagataagtatgcatctgctgctggtgagt |
| | ttcctaatgctgattatcccttggcatatgtacaggtagaccacactccactagatattgaaattgtt |
| | gatgatgaatttagagaggcgattggacgaccatttctaacattagctattgatataaatactcgaa |
| | tggtggtcggttattacttgtccttagaagcccccagtgcaacatctgttgccatgtgtattgcttcc |
| | agtattttgtcaaaaaaacggaagctaattgaattggatatagatgctgattggcaagttgaaggt |
| | cttatggattctatacatacagataatggtcctgactttagaactaaccatatcagtcgagcttgctt |
| | aaaatatgggattcattgggagtttcgtccgattggtggggcaaggtttggtggacatatcgaac |
| | gtttgattggtgttgttaacattgaaatgcacatcttggatggtacaaccttctcgaatatccaagaa |
| | cgtggaacctataattcagcaggtcatgcctgtatgacactaaaagaactagaatattatattgttt |
| | attggatcactaaagtttatcaccaaaagaaacactccaaactcggtatgtcacctttacagaagt |
| | ggcaagatggtgtttggggaacaaaaactagtgttggaacaggtttaaaagagagagtttcgga |
| | tgaagatacattatttattgatttccttcccgaatttgaatcaacaattcaacgtgtaggtgtacaga |
| | aagacaatctattttattttgctgattgtttacgacaatgggttaattgtgttgatcccgaagatgtaa |
| | ataaaaagagaaaaaagaaatttttgtttaaacgtgatccaagggatatttctcttatttggttttatg |
| | aacctctgtccaatacatattttaaagtgcctactgctaagcgagaaataccaccaattagcttattt |
| | gaatacaaacaagtgcagaaatatcttcgtggcgagcaacatgataatcaaaatcaggatgaga |
| | tttatcaggcaattcttcatttacgagagcaattgaaacaatcccgtagtctgactcggaagcaac |
| | ggagagtaaatcaacgtaaaaaggaaaatgcaaaagcaacacatcatttatctgtaaaaaatga |
| | acctgaaaaaccatctattgtcgaaaacagtgttcagccgactaatgatttatggagtattccattg |
| | acggcatttgaagatttaaggggataatagtattctacttttcataggaaaatagccatgcaccaa |
| | gtagaacaacgttggtatattcgtacccctattcaagagggtgaagtattttcctcttggctcatac |
| | gttctgctttggatttaggctgctcaccattaactttagtgaatcttttgtgggaaaaatggagagg |
| | attaacgatagatattgatcgtgggttaaatgaagaaaaattagacatattgttaaatcattgttttgt |
| | ggaacagcatgaagttcagaaaactatgttaagcagttatttccctgctttgattccaaaaaattat |
| | aacaaaaatcagcgtattccttggatattggctctaggtattcgaaatcgttcaaatgtgtcaggtc |
| | gtcaaatttgtacgcaatgtctaagttctacccatacaccaccatatttgcgtatcaagtggcgtat |
| | ggggtggcatagtggctgtgatattcataaaaccatattaatagatcattgtcctcaatgcagaat |
| | cacattacaacctataaaaattgatatagagcatggatcattagcaatttgtacttcatgctatgctg |
| | atttaagtacactcattcaacgtccagtagaaacggatgcattaagatttcagcatcaagctgatc |
| | aggtcttagagtcacaatttgggcgatataaccaaaaggatatttcatcaatagagtggtttgaga |
| | ttgctcgtatatggttaagttttattcgatcccctttaaatatgaagagtcctaaccttttgcaaatgt |
| | ttgaaagtcttaatattgatttaaatatagattatcctattacaccagtagcctttgaatttctgtctg |
| | ttcaggagcgtgaaaaattattatcaattttaaataaaatcatggcattaccttgtgatttatttgttg |
| | atcgctctttagaatatcatgtaagtcattcttatttttgggataaaagaaaaaaactgcctacgcagc |
| | ttcaactaatgaaagataaaatgttgaagccaattaagattattttgccgaaagataacgccatagcc |
| | atgagtaaacccaaatctaaaaaaagtgtacaacggaagtggttaaggctattgagaaagctac |
| | agaaaggggggaacaggtgactttagctcaattttcccataaagaaggctgtagtggttgtgac |
| | agggctgatatagataaattcaaaaaaagatataagggcgataagtattgttcaacttgttatgcc |
| | cgaatttttaaaaaaagagcttgtccaaaatgtggtgaatatgccagattgccaaaaaatgatgat |
| | caagcaatttgtaatgaatgcatcaaaaaacagccttgtattcgctgtaatcaaactaagaagcc |
| | gatagggacactcaccgaatatggtgtggtttgtaatagctgttctgtttattttcgcccaattgagc |
| | gttgtgagaggtgtgatacaccatcacaaaaattgacacatatttcacgatttggtgatgatttaag |
| | ggtatgtcccaaatgctctactagagattatgaaacatgtccctcttgtcataaatatcgcttattag |
| | agcaagatgatacaggagctaagatttgtaaaaaatgtaggaataacgccaaaaagaaatgca |
| | agcaatgtgatgtcttgataccagcaggctgtcctgatctatgtaacaactgttactggcataaga |
| | atctatgggagaaagctagacgaaatattaaagcattccaatcccaacacttacaggctcaatat |
| | gagcaatatttaatttggttagaagatgaggttggtgcaaataaagcagcactgtatatcaataaa |
| | catacccatttttttatcaaaactgaagaattatggcttgatgcaattcctacagcagaacagttatt |
| | ggctgttttaagaaccagtggacttagaaaatttgagttggttgcatcttggctagatgaagccca |
| | tcacattaaggttgcattagaggataaagacttttgctctcaacaggatcagatagaaaaactgat |
| | ttcaagccttcctcacccctctactgcttatgatgtggttatatcttataaagatgagcttgatataaa |
| | aatgaaagatggaaagaccagtatccggtccataaaactggcaataaagcctgcggttgctctt |
| | atgcactatgtttgtgcatcgggagctacattacctaatttaaaccatataaaagcctatcttatcga |
| | cttttctggtcaggctgctgcactaacagggttcattaattttttgaataaaaattttgatacatctat |
| | agactatttggcttttaaaaaatcaaaaaatttcaatgaaaaacgaaaaaataaagtagaaaaaga |
| | aattgtccaatgggtggataagcctttggagaataaggaagatgtattaaattgggtgaagaatg |
| | ggctaagatattttcataatgtatcttatgtggagtctctaaaagtcaaatttgagatgattacagaa |
| | gctgatgatggatatgaaattttattgcagaatcattcttattggttgccaaaaaacacaggagattt |
| | aaaaagatagtttttttgtgattatactgtgctatttcaagctaatttattgtacaagttggtttgttt |
| | atctaattcttctaaaagaactgtccataacaatatggggcaggctgctgccaccgctgagcaataac |
| | tagcataaccccttggggcctctaaacgggtcttgaggggttttttgctgaaacctcaggcatttg |
| | agaagcacacggtcacactgcttccggtagtcaataaaccggtaaaccagcaatagacataag |
| | cggctatttaacgaccctgccctgaaccgacgaccgggtcatcgtggccggatcttgcggccc |
| | ctcggcttgaacgaattgttagacattatttgccgactaccttggtgatctcgcctttcacgtagtg |
| | gacaaattcttccaactgatctgcgcgcgaggccaagcgatcttcttcttgtccaagataagcct |
| | gtctagcttcaagtatgacgggctgatactgggccggcaggcgctccattgcccagtcggcag |
| | cgacatccttcggcgcgattttgccggttactgcgctgtaccaaatgcgggacaacgtaagcac |
| | tacatttcgctcatcgccagcccagtcgggcggcgagttccatagcgttaaggtttcatttagcg |
| | cctcaaatagatcctgttcaggaaccggatcaaagagttcctccgccgctggacctaccaaggc |
| | aacgctatgttctcttgcttttgtcagcaagatagccagatcaatgtcgatcgtggctggctcgaa |
| | gatacctgcaagaatgtcattgcgctgccattctccaaattgcagttcgcgcttagctggataacg |
| | ccacggaatgatgtcgtcgtgcacaacaatggtgacttctacagcgcggagaatctcgctctct |
| | ccaggggaagccgaagtttccaaaaggtcgttgatcaaagctcgccgcgttgtttcatcaagcc |
| | ttacggtcaccgtaaccagcaaatcaatatcactgtgtggcttcaggccgccatccactgcgga |
| | gccgtacaaatgtacggccagcaacgtcggttcgagatggcgctcgatgacgccaactacctc |
| | tgatagttgagtcgatacttcggcgatcaccgcttccctcatactcttcctttttcaatattattgaag |
| | catttatcagggttattgtctcatgagcggatacatatttgaatgtatttagaaaaataaacaaatag |
| | ctagctcactcggtcgctacgctccgggcgtgagactgcggcgggcgctgcggacacataca |
| | aagttacccacagattccgtggataagcaggggactaacatgtgaggcaaaacagcagggcc |
| | gcgccggtggcgtttttccataggctccgccctcctgccagagttcacataaacagacgcttttc |
| | cggtgcatctgtgggagccgtgaggctcaaccatgaatctgacagtacgggcgaaacccgac |
| | aggacttaaagatccccaccgtttccggcgggtcgctccctcttgcgctctcctgttccgaccct |
| | gccgtttaccggatacctgttccgcctttctcccttacgggaagtgtggcgctttctcatagctcac |
| | acactggtatctcggctcggtgtaggtcgttcgctccaagctgggctgtaagcaagaactcccc |
| | gttcagcccgactgctgcgccttatccggtaactgttcacttgagtccaacccggaaaagcacg |
| | gtaaaacgccactggcagcagccattggtaactgggagttcgcagaggatttgtttagctaaac |
| | acgcggttgctcttgaagtgtgcgccaaagtccggctacactggaaggacagatttggttgctg |
| | tgctctgcgaaagccagttaccacggttaagcagttccccaactgacttaaccttcgatcaaacc |
| | acctccccaggtggttttttcgtttacagggcaaaagattacgcgcagaaaaaaaggatctcaag |
| | aagatcctttgatcttttctactgaaccgctctagatttcagtgcaatttatctcttcaaatgtagcac |
| | ctgaagtcagccccatacgatataagttgtaattctcatgttagtcatgccccgcgcccaccgga |
| | aggagctgactgggttgaaggctctcaagggcatcggtcgagatcccggtgcctaatgagtga |
| | gctaacttacattaattgcgttgcgctcactgcccgctttccagtcgggaaacctgtcgtgccagc |
| | tgcattaatgaatcggccaacgcgcggggagaggcggtttgcgtattgggcgccagggtggtt |
| | tttcttttcaccagtgagacgggcaacagctgattgcccttcaccgcctggccctgagagagttg |
| | cagcaagcggtccacgctggtttgccccagcaggcgaaaatcctgtttgatggtggttaacgg |
| | cgggatataacatgagctgtcttcggtatcgtcgtatcccactaccgagatgtccgcaccaacgc |
| | gcagcccggactcggtaatggcgcgcattgcgcccagcgccatctgatcgttggcaaccagc |
| | atcgcagtgggaacgatgccctcattcagcatttgcatggtttgttgaaaaccggacatggcact |
| | ccagtcgccttcccgttccgctatcggctgaatttgattgcgagtgagatatttatgccagccagc |
| | cagacgcagacgcgccgagacagaacttaatgggcccgctaacagcgcgatttgctggtgac |
| | ccaatgcgaccagatgctccacgcccagtcgcgtaccgtcttcatgggagaaaataatactgtt |
| | gatgggtgtctggtcagagacatcaagaaataacgccggaacattagtgcaggcagcttccac |
| | agcaatggcatcctggtcatccagcggatagttaatgatcagcccactgacgcgttgcgcgag |
| | aagattgtgcaccgccgctttacaggcttcgacgccgcttcgttctaccatcgacaccaccacg |
| | ctggcacccagttgatcggcgcgagatttaatcgccgcgacaatttgcgacggcgcgtgcagg |
| | gccagactggaggtggcaacgccaatcagcaacgactgtttgcccgccagttgttgtgccacg |
| | cggttgggaatgtaattcagctccgccatcgccgcttccactttttcccgcgttttcgcagaaacg |
| | tggctggcctggttcaccacgcgggaaacggtctgataagagacaccggcatactctgcgaca |
| | tcgtataacgttactggtttcacattcaccaccctgaattgactctcttccgggcgctatcatgccat |
| | accgcgaaaggttttgcgccattcgatggtgtccgggatctcgacgctctcccttatgcgactcc |
| | tgcattaggaaattaatacgactcactata (SEQ ID NO: 103) |
| pHelper(AjTn6022)_ | ggggaattgtgagcggataacaattcccctgtagaaataattttgtttaactttaataaggagatat |
| ΔTnsD | acatgaaaagaatcagtgatgaatattccccagttaggaaatcccaaactgtatatgggagtattt |
| | cagtaaattatgcttttagaggggaaaagacgatttggtgtgaatctactcttgagcgtgattttttg |
| | attaggcaagaattcaatagcaatgtcatagatatcgtaagccagcctattaccatcccatatata |
| | acagagttgggtaatgaatccacttatactcctgatttcctcgtacagttttccagtgcaaattgtgg |
| | tgattttgaggaaattccctgtccactgttaatagaaataaaacctagaaagaaacttatagaagat |
| | tgggataaattaaagccaaaatttaaggcggctcatacttttgctaaagaaaaaggttggaaattc |
| | aagattataaatgaatcaaggttatatgatcagtattgggaaaatattaattttttgaaaagatttaga |
| | cgttctcatgtggatgtctatgatgaacaaactttaattgagactttagatagtcttggtcaatcgac |
| | tattaatcaattaccggcacatatttttaaggataaaactaatattcttcgaggtattcatcaagtatg |
| | ggcattggtcgcaaaacgaaaaatcatgtgtgatttatattgccccttaacagcagagacggtga |
| | tttggacaaatcaagatgatgtgcttacaaggagatttttaaatggttcagactgatactgtaagta |
| | ttgaccgaagtcggatcatactcaaaccaaatgtaattgttaagtatgaagggcaaccgtataag |
| | attgctaatgttttgaatgccaacgatatcgtgatttctagtttagattctgtgcgttgtttacaagta |
| | agtgcagattctctacaagtatttgaagctgaaaataaccaaactaaagatataaataaaggtgatt |
| | gggatattacgcatgaagcgtggaaaatagctttacatcgttatgaaatcatcaagccgttgatta |
| | agtattcaacaaccgaattagtcgagcaacgagcaagtgaatatggcattaaccgttccacctta |
| | tggaaatggttaaaagattttagagaaaataattctatcttagtgttagttcctaaaaagagaggtt |
| | ggacgaccaagaagtctaggttgtctcctcaaattatgaatattattaaccaaggtattcaagagg |
| | aatatctcaatgccagaaagcctagtattgcaaaaactatagattttattaaagcagaatgttctcg |
| | ccttaaactagaagcaccccatgacaattcaattcgtagaaaaatagaagcattaaacgattatat |
| | ggtgattaaggctcgtcatggttctaaagcagcacaagataagtatgcatctgctgctggtgagt |
| | ttcctaatgctgattatcccttggcatatgtacaggtagaccacactccactagatattgaaattgtt |
| | gatgatgaatttagagaggcgattggacgaccatttctaacattagctattgatataaatactcgaa |
| | tggtggtcggttattacttgtccttagaagcccccagtgcaacatctgttgccatgtgtattgcttcc |
| | agtattttgtcaaaaaaacggaagctaattgaattggatatagatgctgattggcaagttgaaggt |
| | cttatggattctatacatacagataatggtcctgactttagaactaaccatatcagtcgagcttgctt |
| | aaaatatgggattcattgggagtttcgtccgattggtggggcaaggtttggtggacatatcgaac |
| | gtttgattggtgttgttaacattgaaatgcacatcttggatggtacaaccttctcgaatatccaagaa |
| | cgtggaacctataattcagcaggtcatgcctgtatgacactaaaagaactagaatattatattgttt |
| | attggatcactaaagtttatcaccaaaagaaacactccaaactcggtatgtcacctttacagaagt |
| | ggcaagatggtgtttggggaacaaaaactagtgttggaacaggtttaaaagagagagtttcgga |
| | tgaagatacattatttattgatttccttcccgaatttgaatcaacaattcaacgtgtaggtgtacaga |
| | aagacaatctattttattttgctgattgtttacgacaatgggttaattgtgttgatcccgaagatgtaa |
| | ataaaaagagaaaaaagaaatttttgtttaaacgtgatccaagggatatttctcttatttggttttatg |
| | aacctctgtccaatacatattttaaagtgcctactgctaagcgagaaataccaccaattagcttattt |
| | gaatacaaacaagtgcagaaatatcttcgtggcgagcaacatgataatcaaaatcaggatgaga |
| | tttatcaggcaattcttcatttacgagagcaattgaaacaatcccgtagtctgactcggaagcaac |
| | ggagagtaaatcaacgtaaaaaggaaaatgcaaaagcaacacatcatttatctgtaaaaaatga |
| | acctgaaaaaccatctattgtcgaaaacagtgttcagccgactaatgatttatggagtattccattg |
| | acggcatttgaagatttaaggggataataatgcaaaaatatgaacatcttgcagatgatgtacttg |
| | aagtcatgtctctaagtgatagtgatcgtatcaatattttattttctgatcgttggattggttataaaa |
| | aagcggtcactattttggatactttaactgatctgttgaatcgccctaggaagcttcgcccagaatgt |
| | ttattgattgtgggtgattctaatatgggtaaaacgaccattatccacgaattcacaaagcagcatt |
| | acaccaaaacagtgaatgacatagatatggagctgctgtctgtaaaaaaacctgttttaaccatat |
| | tagcccctgctaaagcgaatgtaaaagaattctatatcaatatattgaatcatttctttgtcccatttc |
| | gtcctactgatcctgaagcaaagttacggaatcaagctgtccatctaatgcggaagtatgaaaca |
| | aaaatgttgattattgatgaaatccataattgtttaacaggttctaccaaacttctttccgaagtgatg |
| | aataccttaaagaatctaagtaatgatctaagtctgaatatcgttggcgtcggtactcgtgaagca |
| | gttacgattctacataccgatgctcaatatgcaagccgttttgatgtagcaatgttgcctaaatggg |
| | aactgaatcaggattttctaagactattattaagttatgtacgattattgcctttaaagaaacgttctg |
| | atctagcatctcaaagacttgccacccttatatttgaaatttcaggtgggaattttggtgacttaaac |
| | agattgttgattgagtgtgctaaagaggcaatcgtaaaaggtaatgaagaaattaccgaagaaat |
| | tgttaataaatttaaatggcttaaaccaaccgagggtatgaggaatattagaaatataaatcttaac |
| | atggtttaggtattctacttttcataggaaaatagccgtggttaaggctattgagaaagctacagaa |
| | aggggggaacaggtgactttagctcaattttcccataaagaaggctgtagtggttgtgacaggg |
| | ctgatatagataaattcaaaaaaagatataagggcgataagtattgttcaacttgttatgcccgaat |
| | ttttaaaaaaagagcttgtccaaaatgtggtgaatatgccagattgccaaaaaatgatgatcaagc |
| | aatttgtaatgaatgcatcaaaaaacagccttgtattcgctgtaatcaaactaagaagccgatagg |
| | gacactcaccgaatatggtgtggtttgtaatagctgttctgtttattttcgcccaattgagcgttgtg |
| | agaggtgtgatacaccatcacaaaaattgacacatatttcacgatttggtgatgatttaagggtat |
| | gtcccaaatgctctactagagattatgaaacatgtccctcttgtcataaatatcgcttattagagca |
| | agatgatacaggagctaagatttgtaaaaaatgtaggaataacgccaaaaagaaatgcaagca |
| | atgtgatgtcttgataccagcaggctgtcctgatctatgtaacaactgttactggcataagaatcta |
| | tgggagaaagctagacgaaatattaaagcattccaatcccaacacttacaggctcaatatgagc |
| | aatatttaatttggttagaagatgaggttggtgcaaataaagcagcactgtatatcaataaacatac |
| | ccatttttttatcaaaactgaagaattatggcttgatgcaattcctacagcagaacagttattggctg |
| | ttttaagaaccagtggacttagaaaatttgagttggttgcatcttggctagatgaagcccatcacat |
| | taaggttgcattagaggataaagacttttgctctcaacaggatcagatagaaaaactgatttcaag |
| | ccttcctcacccctctactgcttatgatgtggttatatcttataaagatgagcttgatataaaaatgaa |
| | agatggaaagaccagtatccggtccataaaactggcaataaagcctgcggttgctcttatgcact |
| | atgtttgtgcatcgggagctacattacctaatttaaaccatataaaagcctatcttatcgacttttctg |
| | gtcaggctgctgcactaacagggttcattaattttttgaataaaaattttgatacatctatagactatt |
| | tggcttttaaaaaatcaaaaaatttcaatgaaaaacgaaaaaataaagtagaaaaagaaattgtc |
| | caatgggtggataagcctttggagaataaggaagatgtattaaattgggtgaagaatgggctaa |
| | gatattttcataatgtatcttatgtggagtctctaaaagtcaaatttgagatgattacagaagctgat |
| | gatggatatgaaattttattgcagaatcattcttattggttgccaaaaaacacaggagatttaaaaa |
| | gatagtttttttgtgattatactgtgctatttcaagctaatttattgtacaagttggtttgtttatcta |
| | attcttctaaaagaactgtccataacaatatggggcaggctgctgccaccgctgagcaataactagca |
| | taaccccttggggcctctaaacgggtcttgaggggttttttgctgaaacctcaggcatttgagaa |
| | gcacacggtcacactgcttccggtagtcaataaaccggtaaaccagcaatagacataagcggc |
| | tatttaacgaccctgccctgaaccgacgaccgggtcatcgtggccggatcttgcggcccctcg |
| | gcttgaacgaattgttagacattatttgccgactaccttggtgatctcgcctttcacgtagtggaca |
| | aattcttccaactgatctgcgcgcgaggccaagcgatcttcttcttgtccaagataagcctgtcta |
| | gcttcaagtatgacgggctgatactgggccggcaggcgctccattgcccagtcggcagcgac |
| | atccttcggcgcgattttgccggttactgcgctgtaccaaatgcgggacaacgtaagcactacat |
| | ttcgctcatcgccagcccagtcgggcggcgagttccatagcgttaaggtttcatttagcgcctca |
| | aatagatcctgttcaggaaccggatcaaagagttcctccgccgctggacctaccaaggcaacg |
| | ctatgttctcttgcttttgtcagcaagatagccagatcaatgtcgatcgtggctggctcgaagatac |
| | ctgcaagaatgtcattgcgctgccattctccaaattgcagttcgcgcttagctggataacgccac |
| | ggaatgatgtcgtcgtgcacaacaatggtgacttctacagcgcggagaatctcgctctctccag |
| | gggaagccgaagtttccaaaaggtcgttgatcaaagctcgccgcgttgtttcatcaagccttacg |
| | gtcaccgtaaccagcaaatcaatatcactgtgtggcttcaggccgccatccactgcggagccgt |
| | acaaatgtacggccagcaacgtcggttcgagatggcgctcgatgacgccaactacctctgata |
| | gttgagtcgatacttcggcgatcaccgcttccctcatactcttcctttttcaatattattgaagcattt |
| | atcagggttattgtctcatgagcggatacatatttgaatgtatttagaaaaataaacaaatagctagc |
| | tcactcggtcgctacgctccgggcgtgagactgcgggggcgctgcggacacatacaaagtt |
| | acccacagattccgtggataagcaggggactaacatgtgaggcaaaacagcagggccgcgc |
| | cggtggcgtttttccataggctccgccctcctgccagagttcacataaacagacgcttttccggtg |
| | catctgtgggagccgtgaggctcaaccatgaatctgacagtacgggcgaaacccgacaggac |
| | ttaaagatccccaccgtttccggcgggtcgctccctcttgcgctctcctgttccgaccctgccgttt |
| | accggatacctgttccgcctttctcccttacgggaagtgtggcgctttctcatagctcacacactg |
| | gtatctcggctcggtgtaggtcgttcgctccaagctgggctgtaagcaagaactccccgttcag |
| | cccgactgctgcgccttatccggtaactgttcacttgagtccaacccggaaaagcacggtaaaa |
| | cgccactggcagcagccattggtaactgggagttcgcagaggatttgtttagctaaacacgcg |
| | gttgctcttgaagtgtgcgccaaagtccggctacactggaaggacagatttggttgctgtgctct |
| | gcgaaagccagttaccacggttaagcagttccccaactgacttaaccttcgatcaaaccacctc |
| | cccaggtggttttttcgtttacagggcaaaagattacgcgcagaaaaaaaggatctcaagaaga |
| | tcctttgatcttttctactgaaccgctctagatttcagtgcaatttatctcttcaaatgtagcacctga |
| | agtcagccccatacgatataagttgtaattctcatgttagtcatgccccgcgcccaccggaaggag |
| | ctgactgggttgaaggctctcaagggcatcggtcgagatcccggtgcctaatgagtgagctaa |
| | cttacattaattgcgttgcgctcactgcccgctttccagtcgggaaacctgtcgtgccagctgcat |
| | taatgaatcggccaacgcgcggggagaggcggtttgcgtattgggcgccagggtggtttttctt |
| | ttcaccagtgagacgggcaacagctgattgcccttcaccgcctggccctgagagagttgcagc |
| | aagcggtccacgctggtttgccccagcaggcgaaaatcctgtttgatggtggttaacggcggg |
| | atataacatgagctgtcttcggtatcgtcgtatcccactaccgagatgtccgcaccaacgcgcag |
| | cccggactcggtaatggcgcgcattgcgcccagcgccatctgatcgttggcaaccagcatcgc |
| | agtgggaacgatgccctcattcagcatttgcatggtttgttgaaaaccggacatggcactccagt |
| | cgccttcccgttccgctatcggctgaatttgattgcgagtgagatatttatgccagccagccagac |
| | gcagacgcgccgagacagaacttaatgggcccgctaacagcgcgatttgctggtgacccaat |
| | gcgaccagatgctccacgcccagtcgcgtaccgtcttcatgggagaaaataatactgttgatgg |
| | gtgtctggtcagagacatcaagaaataacgccggaacattagtgcaggcagcttccacagcaa |
| | tggcatcctggtcatccagcggatagttaatgatcagcccactgacgcgttgcgcgagaagatt |
| | gtgcaccgccgctttacaggcttcgacgccgcttcgttctaccatcgacaccaccacgctggca |
| | cccagttgatcggcgcgagatttaatcgccgcgacaatttgcgacggcgcgtgcagggccag |
| | actggaggtggcaacgccaatcagcaacgactgtttgcccgccagttgttgtgccacgcggtt |
| | gggaatgtaattcagctccgccatcgccgcttccactttttcccgcgttttcgcagaaacgtggct |
| | ggcctggttcaccacgcgggaaacggtctgataagagacaccggcatactctgcgacatcgta |
| | taacgttactggtttcacattcaccaccctgaattgactctcttccgggcgctatcatgccataccg |
| | cgaaaggttttgcgccattcgatggtgtccgggatctcgacgctctcccttatgcgactcctgcat |
| | taggaaattaatacgactcactata (SEQ ID NO: 104) |
| pHelper(AjTn6022)_ | ggggaattgtgagcggataacaattcccctgtagaaataattttgtttaactttaataaggagatat |
| ΔTnsF | acatgaaaagaatcagtgatgaatattccccagttaggaaatcccaaactgtatatgggagtattt |
| | cagtaaattatgcttttagaggggaaaagacgatttggtgtgaatctactcttgagcgtgattttttg |
| | attaggcaagaattcaatagcaatgtcatagatatcgtaagccagcctattaccatcccatatata |
| | acagagttgggtaatgaatccacttatactcctgatttcctcgtacagttttccagtgcaaattgtgg |
| | tgattttgaggaaattccctgtccactgttaatagaaataaaacctagaaagaaacttatagaagat |
| | tgggataaattaaagccaaaatttaaggcggctcatacttttgctaaagaaaaaggttggaaattc |
| | aagattataaatgaatcaaggttatatgatcagtattgggaaaatattaattttttgaaaagatttaga |
| | cgttctcatgtggatgtctatgatgaacaaactttaattgagactttagatagtcttggtcaatcgac |
| | tattaatcaattaccggcacatatttttaaggataaaactaatattcttcgaggtattcatcaagtatg |
| | ggcattggtcgcaaaacgaaaaatcatgtgtgatttatattgccccttaacagcagagacggtga |
| | tttggacaaatcaagatgatgtgcttacaaggagatttttaaatggttcagactgatactgtaagta |
| | ttgaccgaagtcggatcatactcaaaccaaatgtaattgttaagtatgaagggcaaccgtataag |
| | attgctaatgttttgaatgccaacgatatcgtgatttctagtttagattctgtgcgttgtttacaagta |
| | agtgcagattctctacaagtatttgaagctgaaaataaccaaactaaagatataaataaaggtgatt |
| | gggatattacgcatgaagcgtggaaaatagctttacatcgttatgaaatcatcaagccgttgatta |
| | agtattcaacaaccgaattagtcgagcaacgagcaagtgaatatggcattaaccgttccacctta |
| | tggaaatggttaaaagattttagagaaaataattctatcttagtgttagttcctaaaaagagaggtt |
| | ggacgaccaagaagtctaggttgtctcctcaaattatgaatattattaaccaaggtattcaagagg |
| | aatatctcaatgccagaaagcctagtattgcaaaaactatagattttattaaagcagaatgttctcg |
| | ccttaaactagaagcaccccatgacaattcaattcgtagaaaaatagaagcattaaacgattatat |
| | ggtgattaaggctcgtcatggttctaaagcagcacaagataagtatgcatctgctgctggtgagt |
| | ttcctaatgctgattatcccttggcatatgtacaggtagaccacactccactagatattgaaattgtt |
| | gatgatgaatttagagaggcgattggacgaccatttctaacattagctattgatataaatactcgaa |
| | tggtggtcggttattacttgtccttagaagcccccagtgcaacatctgttgccatgtgtattgcttcc |
| | agtattttgtcaaaaaaacggaagctaattgaattggatatagatgctgattggcaagttgaaggt |
| | cttatggattctatacatacagataatggtcctgactttagaactaaccatatcagtcgagcttgctt |
| | aaaatatgggattcattgggagtttcgtccgattggtggggcaaggtttggtggacatatcgaac |
| | gtttgattggtgttgttaacattgaaatgcacatcttggatggtacaaccttctcgaatatccaagaa |
| | cgtggaacctataattcagcaggtcatgcctgtatgacactaaaagaactagaatattatattgttt |
| | attggatcactaaagtttatcaccaaaagaaacactccaaactcggtatgtcacctttacagaagt |
| | ggcaagatggtgtttggggaacaaaaactagtgttggaacaggtttaaaagagagagtttcgga |
| | tgaagatacattatttattgatttccttcccgaatttgaatcaacaattcaacgtgtaggtgtacaga |
| | aagacaatctattttattttgctgattgtttacgacaatgggttaattgtgttgatcccgaagatgtaa |
| | ataaaaagagaaaaaagaaatttttgtttaaacgtgatccaagggatatttctcttatttggttttatg |
| | aacctctgtccaatacatattttaaagtgcctactgctaagcgagaaataccaccaattagcttattt |
| | gaatacaaacaagtgcagaaatatcttcgtggcgagcaacatgataatcaaaatcaggatgaga |
| | tttatcaggcaattcttcatttacgagagcaattgaaacaatcccgtagtctgactcggaagcaac |
| | ggagagtaaatcaacgtaaaaaggaaaatgcaaaagcaacacatcatttatctgtaaaaaatga |
| | acctgaaaaaccatctattgtcgaaaacagtgttcagccgactaatgatttatggagtattccattg |
| | acggcatttgaagatttaaggggataataatgcaaaaatatgaacatcttgcagatgatgtacttg |
| | aagtcatgtctctaagtgatagtgatcgtatcaatattttattttctgatcgttggattggttataaaa |
| | aagcggtcactattttggatactttaactgatctgttgaatcgccctaggaagcttcgcccagaatgt |
| | ttattgattgtgggtgattctaatatgggtaaaacgaccattatccacgaattcacaaagcagcatt |
| | acaccaaaacagtgaatgacatagatatggagctgctgtctgtaaaaaaacctgttttaaccatat |
| | tagcccctgctaaagcgaatgtaaaagaattctatatcaatatattgaatcatttctttgtcccatttc |
| | gtcctactgatcctgaagcaaagttacggaatcaagctgtccatctaatgcggaagtatgaaaca |
| | aaaatgttgattattgatgaaatccataattgtttaacaggttctaccaaacttctttccgaagtgatg |
| | aataccttaaagaatctaagtaatgatctaagtctgaatatcgttggcgtcggtactcgtgaagca |
| | gttacgattctacataccgatgctcaatatgcaagccgttttgatgtagcaatgttgcctaaatggg |
| | aactgaatcaggattttctaagactattattaagttatgtacgattattgcctttaaagaaacgttctg |
| | atctagcatctcaaagacttgccacccttatatttgaaatttcaggtgggaattttggtgacttaaac |
| | agattgttgattgagtgtgctaaagaggcaatcgtaaaaggtaatgaagaaattaccgaagaaat |
| | tgttaataaatttaaatggcttaaaccaaccgagggtatgaggaatattagaaatataaatcttaac |
| | atggtttaggtattctacttttcataggaaaatagccatgcaccaagtagaacaacgttggtatatt |
| | cgtacccctattcaagagggtgaagtattttcctcttggctcatacgttctgctttggatttaggctg |
| | ctcaccattaactttagtgaatcttttgtgggaaaaatggagaggattaacgatagatattgatcgt |
| | gggttaaatgaagaaaaattagacatattgttaaatcattgttttgtggaacagcatgaagttcaga |
| | aaactatgttaagcagttatttccctgctttgattccaaaaaattataacaaaaatcagcgtattcctt |
| | ggatattggctctaggtattcgaaatcgttcaaatgtgtcaggtcgtcaaatttgtacgcaatgtct |
| | aagttctacccatacaccaccatatttgcgtatcaagtggcgtatggggggcatagtggctgtg |
| | atattcataaaaccatattaatagatcattgtcctcaatgcagaatcacattacaacctataaaaatt |
| | gatatagagcatggatcattagcaatttgtacttcatgctatgctgatttaagtacactcattcaacg |
| | tccagtagaaacggatgcattaagatttcagcatcaagctgatcaggtcttagagtcacaatttgg |
| | gcgatataaccaaaaggatatttcatcaatagagtggtttgagattgctcgtatatggttaagtttta |
| | ttcgatcccctttaaatatgaagagtcctaaccttttgcaaatgtttgaaagtcttaatattgatttaa |
| | atatagattatcctattacaccagtagcctttgaatttctgtctgttcaggagcgtgaaaaattattat |
| | caattttaaataaaatcatggcattaccttgtgatttatttgttgatcgctctttagaatatcatgtaa |
| | gtcattcttatttttgggataaaagaaaaaaactgcctacgcagcttcaactaatgaaagataaaatgt |
| | tgaagccaattaagattattttgccgaaagataacgccatagccatgagtaaacccaaatctaaaa |
| | aaagtgtacaacggaagtggttaaggctattgagaaagctacagaaaggggggaacaggtga |
| | tttttttgtgattatactgtgctatttcaagctaatttattgtacaagttggtttgtttatctaattct |
| | tctaaaagaactgtccataacaatatggggcaggctgctgccaccgctgagcaataactagcataacc |
| | ccttggggcctctaaacgggtcttgaggggttttttgctgaaacctcaggcatttgagaagcaca |
| | cggtcacactgcttccggtagtcaataaaccggtaaaccagcaatagacataagcggctattta |
| | acgaccctgccctgaaccgacgaccgggtcatcgtggccggatcttgcggcccctcggcttga |
| | acgaattgttagacattatttgccgactaccttggtgatctcgcctttcacgtagtggacaaattctt |
| | ccaactgatctgcgcgcgaggccaagcgatcttcttcttgtccaagataagcctgtctagcttca |
| | agtatgacgggctgatactgggccggcaggcgctccattgcccagtcggcagcgacatccttc |
| | ggcgcgattttgccggttactgcgctgtaccaaatgcgggacaacgtaagcactacatttcgctc |
| | atcgccagcccagtcgggcggcgagttccatagcgttaaggtttcatttagcgcctcaaatagat |
| | cctgttcaggaaccggatcaaagagttcctccgccgctggacctaccaaggcaacgctatgttc |
| | tcttgcttttgtcagcaagatagccagatcaatgtcgatcgtggctggctcgaagatacctgcaa |
| | gaatgtcattgcgctgccattctccaaattgcagttcgcgcttagctggataacgccacggaatg |
| | atgtcgtcgtgcacaacaatggtgacttctacagcgcggagaatctcgctctctccaggggaag |
| | ccgaagtttccaaaaggtcgttgatcaaagctcgccgcgttgtttcatcaagccttacggtcacc |
| | gtaaccagcaaatcaatatcactgtgtggcttcaggccgccatccactgcggagccgtacaaat |
| | gtacggccagcaacgtcggttcgagatggcgctcgatgacgccaactacctctgatagttgagt |
| | cgatacttcggcgatcaccgcttccctcatactcttcctttttcaatattattgaagcatttatcaggg |
| | ttattgtctcatgagcggatacatatttgaatgtatttagaaaaataaacaaatagctagctcactcg |
| | gtcgctacgctccgggcgtgagactgcggcgggcgctgcggacacatacaaagttacccaca |
| | gattccgtggataagcaggggactaacatgtgaggcaaaacagcagggccgcgccggtggc |
| | gtttttccataggctccgccctcctgccagagttcacataaacagacgcttttccggtgcatctgtg |
| | ggagccgtgaggctcaaccatgaatctgacagtacgggcgaaacccgacaggacttaaagat |
| | ccccaccgtttccggcgggtcgctccctcttgcgctctcctgttccgaccctgccgtttaccggat |
| | acctgttccgcctttctcccttacgggaagtgtggcgctttctcatagctcacacactggtatctcg |
| | gctcggtgtaggtcgttcgctccaagctgggctgtaagcaagaactccccgttcagcccgactg |
| | ctgcgccttatccggtaactgttcacttgagtccaacccggaaaagcacggtaaaacgccactg |
| | gcagcagccattggtaactgggagttcgcagaggatttgtttagctaaacacgcggttgctcttg |
| | aagtgtgcgccaaagtccggctacactggaaggacagatttggttgctgtgctctgcgaaagc |
| | cagttaccacggttaagcagttccccaactgacttaaccttcgatcaaaccacctccccaggtgg |
| | ttttttcgtttacagggcaaaagattacgcgcagaaaaaaaggatctcaagaagatcctttgatctt |
| | ttctactgaaccgctctagatttcagtgcaatttatctcttcaaatgtagcacctgaagtcagcccc |
| | atacgatataagttgtaattctcatgttagtcatgccccgcgcccaccggaaggagctgactggg |
| | ttgaaggctctcaagggcatcggtcgagatcccggtgcctaatgagtgagctaacttacattaat |
| | tgcgttgcgctcactgcccgctttccagtcgggaaacctgtcgtgccagctgcattaatgaatcg |
| | gccaacgcgcggggagaggcggtttgcgtattgggcgccagggtggtttttttttcaccagtg |
| | agacgggcaacagctgattgcccttcaccgcctggccctgagagagttgcagcaagcggtcc |
| | acgctggtttgccccagcaggcgaaaatcctgtttgatggtggttaacggcgggatataacatg |
| | agctgtcttcggtatcgtcgtatcccactaccgagatgtccgcaccaacgcgcagcccggactc |
| | ggtaatggcgcgcattgcgcccagcgccatctgatcgttggcaaccagcatcgcagtgggaa |
| | cgatgccctcattcagcatttgcatggtttgttgaaaaccggacatggcactccagtcgccttccc |
| | gttccgctatcggctgaatttgattgcgagtgagatatttatgccagccagccagacgcagacg |
| | cgccgagacagaacttaatgggcccgctaacagcgcgatttgctggtgacccaatgcgacca |
| | gatgctccacgcccagtcgcgtaccgtcttcatgggagaaaataatactgttgatgggtgtctgg |
| | tcagagacatcaagaaataacgccggaacattagtgcaggcagcttccacagcaatggcatcc |
| | tggtcatccagcggatagttaatgatcagcccactgacgcgttgcgcgagaagattgtgcaccg |
| | ccgctttacaggcttcgacgccgcttcgttctaccatcgacaccaccacgctggcacccagttg |
| | atcggcgcgagatttaatcgccgcgacaatttgcgacggcgcgtgcagggccagactggagg |
| | tggcaacgccaatcagcaacgactgtttgcccgccagttgttgtgccacgcggttgggaatgta |
| | attcagctccgccatcgccgcttccactttttcccgcgttttcgcagaaacgtggctggcctggtt |
| | caccacgcgggaaacggtctgataagagacaccggcatactctgcgacatcgtataacgttac |
| | tggtttcacattcaccaccctgaattgactctcttccgggcgctatcatgccataccgcgaaaggt |
| | tttgcgccattcgatggtgtccgggatctcgacgctctcccttatgcgactcctgcattaggaaatt |
| | aatacgactcactata (SEQ ID NO: 105) |
| pTarget(AjTn6022)_ | cggggcggagatttcctggaagatgccaggaagatacttaacagggaagtgagagggccgc |
| U50D50 | ggcaaagccgtttttccataggctccgcccccctgacaagcatcacgaaatctgacgctcaaat |
| | cagtggtggcgaaacccgacaggactataaagataccaggcgtttccccctggcggctccctc |
| | gtgcgctctcctgttcctgcctttcggtttaccggtgtcattccgctgttatggccgcgtttgtctcat |
| | tccacgcctgacactcagttccgggtaggcagttcgctccaagctggactgtatgcacgaaccc |
| | cccgttcagtccgaccgctgcgccttatccggtaactatcgtcttgagtccaacccggaaagac |
| | atgcaaaagcaccactggcagcagccactggtaattgatttagaggagttagtcttgaagtcatg |
| | cgccggttaaggctaaactgaaaggacaagttttggtgactgcgctcctccaagccagttacct |
| | cggttcaaagagttggtagctcagagaaccttcgaaaaaccgccctgcaaggcggttttttcgtt |
| | ttcagagcaagagattacgcgcagaccaaaacgatctcaagaagatcatcttattaatatacgta |
| | gtatctacgatacgtagtatctacgatacgtagatcatggagaagtcatttaataaggccactgtta |
| | aacgtagatatatctacgcgtagatatatctacgatacgtagcggccgacgcgctgggctacgt |
| | ctttaatgggcagcgcccacatacgcagcgatttccaacagcttgaggcggaagctttaatgcg |
| | gtagtttatcacagttaaattgctaacgcagtcaggcaccgtgtatgaaatctaacaatgcgctca |
| | tcgtcatcctcggcaccgtcaccctggatgctgtaggcataggcttggttatgccggtactgccg |
| | ggcctcttgcgggatatcgtccattccgacagcatcgccagtcactatggcgtgctgctagcgc |
| | tatatgcgttgatgcaatttctatgcgcacccgttctcggagcactgtccgaccgctttggccgcc |
| | gcccagtcctgctcgcttcgctacttggagccactatcgactacgcgatcatggcgaccacacc |
| | cgtcctgtggatcctctacgccggacgcatcgtggccggcatcaccggcgccacaggtgcgg |
| | ttgctggcgcctatatcgccgacatcaccgatggggaagatcgggcctgccaccatacccacg |
| | ccgaaacaagcgccctgcaccattatgttccggatctgcatcgcaggatgctgctggctaccct |
| | gtggaacacctacatctgtattaacgaagcgctaaccgtttttatcaggctctgggaggcagaat |
| | aaatgatcatatcgtcaattattacctccacggggagagcctgagcaaactggcctcaggcattt |
| | gagaagcacacggtcacactgcttccggtagtcaataaaccggtaaaccagcaatagacataa |
| | gcggctatttaacgaccctgccctgaaccgacgaccgggtcgaatttgctttcgaatttctgccat |
| | tcatccgcttattatcacttattcaggcgtagcaaccaggcgtttaagggcaccaataactgcctt |
| | aaaaaaattacgccccgccctgccactcatcgcagtactgttgtaattcattaagcattctgccga |
| | catggaagccatcacaaacggcatgatgaacctgaatcgccagcggcatcagcaccttgtcgc |
| | cttgcgtataatatttgcccatggtgaaaacgggggcgaagaagttgtccatattggccacgttta |
| | aatcaaaactggtgaaactcacccagggattggctgagacgaaaaacatattctcaataaaccc |
| | tttagggaaataggccaggttttcaccgtaacacgccacatcttgcgaatatatgtgtagaaactg |
| | ccggaaatcgtcgtggtattcactccagagcgatgaaaacgtttcagtttgctcatggaaaacgg |
| | tgtaacaagggtgaacactatcccatatcaccagctcaccgtctttcattgccatacggaattccg |
| | gatgagcattcatcaggcgggcaagaatgtgaataaaggccggataaaacttgtgcttatttttct |
| | ttacggtctttaaaaaggccgtaatatccagctgaacggtctggttataggtacattgagcaactg |
| | actgaaatgcctcaaaatgttctttacgatgccattgggatatatcaacggtggtatatccagtgat |
| | ttttttctccattttagcttccttagctcctgaaaatctcgataactcaaaaaatacgcccgcatttcg |
| | tgcaccgcaccatacagcttctgcggttgccttagtcggtggcgggtcacaacccaaacccggt |
| | gaaattactttggcacataaaggcgtgctgtagtgatcttatttcattatggtgaaagttggaacct |
| | cttacgtgccgatcaacgtctcattttcgccaaaagttggcccagggcttcccggtatcaacagg |
| | gacaccaggatttatttattctgcgaagtgatcttccgtcacaggtatttattcggcgcaaagtgcg |
| | tcgggtgatgctgccaacttactgatttagtgtatgatggtgtttttgaggtgctccagtggcttctg |
| | tttctatcagctgtccctcctgttcagctactgacggggtggtgcgtaacggcaaaagcaccgcc |
| | ggacatcagcgctagcggagtgtatactggcttactatgttggcactgatgagggtgtcagtga |
| | agtgcttcatgtggcaggagaaaaaaggctgcaccggtgcgtcagcagaatatgtgatacagg |
| | atatattccgcttcctcgctcactgactcgctacgctcggtcgttcgactgcggcgagcggaaat |
| | ggcttacgaa (SEQ ID NO: 106) |
| pUC-Donor(AjTn6022) | tcgcgcgtttcggtgatgacggtgaaaacctctgacacatgcagctcccggagacggtcacag |
| | cttgtctgtaagcggatgccgggagcagacaagcccgtcagggcgcgtcagcgggtgttggc |
| | gggtgtcggggctggcttaactatgcggcatcagagcagattgtactgagagtgcaccatatgc |
| | ggtgtgaaataccgcacagatgcgtaaggagaaaataccgcatcaggcgccattcgccattca |
| | ggctgcgcaactgttgggaagggcgatcggtgcgggcctcttcgctattacgccagctggcga |
| | aagggggatgtgctgcaaggcgattaagttgggtaacgccagggttttcccagtcacgacgtt |
| | gtaaaacgacggccagtgaattgacgcgtattgggatagcacgcctttatgtgccaaagtaattt |
| | caccgggtttgggttgtgacccgctgtcatttacagcaatagataagagtgtcatgataagaaaa |
| | caataggttgccactctaaaattacacaaatagattagagtaatgacagtaataagcaagagtgg |
| | cttgcaaatttacacaaatacgcacgagtggctgtaagaggcattctgtcgtaggtcttgaaagg |
| | agtgggaattggctccggtgcccgtcagtgggcagagcgcacatcgcccacagtccccgaga |
| | agttggggggaggggtcggcaattgatccggtgcctagagaaggtggcgcggggtaaactg |
| | ggaaagtgatgtcgtgtactggctccgcctttttcccgagggtgggggagaaccgtatataagt |
| | gcagtagtcgccgtgaacgttctttttcgcaacgggtttgccgccagaacacaggaccggttct |
| | agagcgctgccaccatgaccgagtacaagcccacggtgcgcctcgccacccgcgacgacgt |
| | ccccagggccgtacgcaccctcgccgccgcgttcgccgactaccccgccacgcgccacacc |
| | gtcgatccggaccgccacatcgagcgggtcaccgagctgcaagaactcttcctcacgcgcgt |
| | cgggctcgacatcggcaaggtgtgggtcgcggacgacggcgccgcggtggcggtctggac |
| | cacgccggagagcgtcgaagcgggggcggtgttcgccgagatcggcccgcgcatggccga |
| | gttgagcggttcccggctggccgcgcagcaacagatggaaggcctcctggcgccgcaccgg |
| | cccaaggagcccgcgtggttcctggccaccgtcggcgtctcgcccgaccaccagggcaagg |
| | gtctgggcagcgccgtcgtgctccccggagtggaggcggccgagcgcgccggggtgcccg |
| | ccttcctggagacctccgcgccccgcaacctccccttctacgagcggctcggcttcaccgtcac |
| | cgccgacgtcgaggtgcccgaaggaccgcgcacctggtgcatgacccgcaagcccggtgc |
| | ctgattctaaataaaggaaatttattttcattgcaatagtgtgttggaattttttgtgtctctcagcac |
| | ttcactgccctcattcctaagaaacccaccatatctatagacagttctgcgaatattatcatttgggaa |
| | acaatcaatagttgtgactttaacttttttatttgcaggcatatcttttagtgaatttaatgaaattca |
| | tatataaatagtatagtttttattatcactctagtttatttgtgtaaactctgatttatttgtggaact |
| | gacacccgccaccgactaaggcaaccgcagaagctgtatggtgcggtgcacgaaatgatcccaatgg |
| | cgcgccgagcttggcgtaatcatggtcatagctgtttcctgtgtgaaattgttatccgctcacaatt |
| | ccacacaacatacgagccggaagcataaagtgtaaagcctggggtgcctaatgagtgagcta |
| | actcacattaattgcgttgcgctcactgcccgctttccagtcgggaaacctgtcgtgccagctgc |
| | attaatgaatcggccaacgcgcggggagaggcggtttgcgtattgggcgctcttccgcttcctc |
| | gctcactgactcgctgcgctcggtcgttcggctgcggcgagcggtatcagctcactcaaaggc |
| | ggtaatacggttatccacagaatcaggggataacgcaggaaagaacatgtgagcaaaaggcc |
| | agcaaaaggccaggaaccgtaaaaaggccgcgttgctggcgtttttccataggctccgccccc |
| | ctgacgagcatcacaaaatcacaaaaatcgacgctcaagtcagaggtggcgaaacccgacag |
| | gactataaagataccaggcgtttccccctggaagctccctcgtgcgctctcctgttccgaccctg |
| | ccgcttaccggatacctgtccgcctttctcccttcgggaagcgtggcgctttctcatagctcacgc |
| | tgtaggtatctcagttcggtgtaggtcgttcgctccaagctgggctgtgtgcacgaaccccccgt |
| | tcagcccgaccgctgcgccttatccggtaactatcgtcttgagtccaacccggtaagacacgac |
| | ttatcgccactggcagcagccactggtaacaggattagcagagcgaggtatgtaggcggtgct |
| | acagagttcttgaagtggtggcctaactacggctacactagaagaacagtatttggtatctgcgc |
| | tctgctgaagccagttaccttcggaaaaagagttggtagctcttgatccggcaaacaaaccacc |
| | gctggtagcggtggtttttttgtttgcaagcagcagattacgcgcagaaaaaaaggatctcaaga |
| | agatcctttgatcttttctacggggtctgacgctcagtggaacgaaaactcacgttaagggatttt |
| | ggtcatgagattatcaaaaaggatcttcacctagatccttttaaattaaaaatgaagttttaaatcaa |
| | tctaaagtatatatgagtaaacttggtctgacagttaccaatgcttaatcagtgaggcacctatctc |
| | agcgatctgtctatttcgttcatccatagttgcctgactccccgtcgtgtagataactacgatacgg |
| | gagggcttaccatctggccccagtgctgcaatgataccgcgagacccacgctcaccggctcca |
| | gatttatcagcaataaaccagccagccggaagggccgagcgcagaagtggtcctgcaacttta |
| | tccgcctccatccagtctattaattgttgccgggaagctagagtaagtagttcgccagttaatagtt |
| | tgcgcaacgttgttgccattgctacaggcatcgtggtgtcacgctcgtcgtttggtatggcttcatt |
| | cagctccggttcccaacgatcaaggcgagttacatgatcccccatgttgtgcaaaaaagcggtt |
| | agctccttcggtcctccgatcgttgtcagaagtaagttggccgcagtgttatcactcatggttatg |
| | gcagcactgcataattctcttactgtcatgccatccgtaagatgcttttctgtgactggtgagtactc |
| | aaccaagtcattctgagaatagtgtatgcggcgaccgagttgctcttgcccggcgtcaatacgg |
| | gataataccgcgccacatagcagaactttaaaagtgctcatcattggaaaacgttcttcggggcg |
| | aaaactctcaaggatcttaccgctgttgagatccagttcgatgtaacccactcgtgcacccaact |
| | gatcttcagcatcttttactttcaccagcgtttctgggtgagcaaaaacaggaaggcaaaatgcc |
| | gcaaaaaagggaataagggcgacacggaaatgttgaatactcatactcttcctttttcaatattatt |
| | gaagcatttatcagggttattgtctcatgagcggatacatatttgaatgtatttagaaaaataaaca |
| | aataggggttccgcgcacatttccccgaaaagtgccacctgacgtctaagaaaccattattatca |
| | tgacattaacctataaaaataggcgtatcacgaggccctttcgtc (SEQ ID NO: 107) |
| pR6K- | gagagtgcaccatacaggaaacagctatgaccatgattacgccaagctcggcgcgccattgg |
| Donor(AjTn6022) | gattgtcagttccacaaataaatcagagtttacacaaataaactagagtgataataaaaactatact |
| | atttatatatgaatttcattaaattcactaaaagatatgcctgcaaataaaaaagttaaagtcacaac |
| | tattgattgtttcccaaatgataatattcgcagaactgtctatagatatggtgggtttcttaggaatga |
| | gggcagtgaagagcgcttttgaagctgatgtgcttaaaaacttactcaatggaataattctagata |
| | attcactagtgattggaaatgttggatcctcatactcttcctttttcaattcagaagaactcgtcaag |
| | aaggcgatagaaggcgatgcgctgcgaatcgggagcggcgataccgtaaagcacgaggaa |
| | gcggtcagcccattcgccgccaagctcttcagcaatatcacgggtagccaacgctatgtcctga |
| | tagcggtccgccacacccagccggccacagtcgatgaatccagaaaagcggccattttccac |
| | catgatattcggcaagcaggcatcgccatgggtcacgacgagatcctcgccgtcgggcatgc |
| | gcgccttgagcctggcgaacagttcggctggcgcgagcccctgatgctcttcgtccagatcatc |
| | ctgatcgacaagaccggcttccatccgagtacgtgctcgctcgatgcgatgtttcgcttggtggt |
| | cgaatgggcaggtagccggatcaagcgtatgcagccgccgcattgcatcagccatgatggat |
| | actttctcggcaggagcaaggtgggatgacaggagatcctgccccggcacttcgcccaatagc |
| | agccagtcccttcccgcttcagtgacaacgtcgagcacagctgcgcaaggaacgcccgtcgt |
| | ggccagccacgatagccgcgctgcctcgtcctgcagttcattcagggcaccggacaggtcgg |
| | tcttgacaaaaagaaccgggcgcccctgcgctgacagccggaacacggggcatcagagca |
| | gccgattgtctgttgtgcccagtcatagccgaatagcctctccacccaagcggccggagaacct |
| | gcgtgcaatccatcttgttcaatcatgcgaaacgatcctcatcctgtctcttgatcagatcttgatcc |
| | cctgcgccatcagatccttggcggcaagaaagccatccagtttactttgcagggcttcccaacct |
| | taccagagggcgccccagctggcaattccggttcgcttgctgtccataaaaccgcccagtctag |
| | ctatcgccatgtaagcccactgcaagctacctgctttctctttgcgcttgcgttttcccttgtccaga |
| | tagcccagtagctgacattcatccggggtcagcaccgtttctgcggactggctttctacgtgttcc |
| | gcttcctttagcagcccttgcgccatgcatttgttaaatcgaattatccattgctgttgacaaaggg |
| | aatcaggggatcttgaagttcctattccgaagttcctattctctagaaagtataggaacttcagagc |
| | gcttttgaagctaattcgagatcatgcatgagctcactagtggatccccccgacagaatgcctctt |
| | acagccactcgtgcgtatttgtgtaaatttgcaagccactcttgcttattactgtcattactctaatct |
| | atttgtgtaattttagagtggcaacctattgttttcttatcatgacactcttatctattgctgtaaatg |
| | acaatcccaatacgcgtcaattcactggccgtcgttttactactttccgctgcataaccctgcttcggg |
| | gtcattatagcgattttttcggtatatccatcctttttcgcacgatatacaggattttgccaaagggtt |
| | cgtgtagactttccttggtgtatccaacggcgtcagccgggcaggataggtgaagtaggccca |
| | cccgcgagcgggtgttccttcttcactgtcccttattcgcacctggcggtgctcaacgggaatcc |
| | tgctctgcgaggctggccgataagctctgataacgcaggaaagaacatgggcagttcaacctg |
| | ttgatagtacgtactaagctctcatgtttcacgtactaagctctcaagtttaacgtactaagctctcat |
| | gtttaacgaactaaaccctcatggctaacgtactaagctctcatggctaacgtactaagctctcat |
| | gtttcacgtactaagctctcatgtttgaacaataaaattaatataaatcagcaacttaaatagcctct |
| | aaggttttaagttttataagaaaaaaaagaatatataaggcttttaaagcttttaaggtttaacggtt |
| | gtggacaacaagccagggatgtaacgcactgagaagcccttagagcctctcaaagcaattttg |
| | agtgacacaggaacacttaacggctgacaaccggctggtaacaggattagcagagcgaggta |
| | tgtaggcggtgctacaagattgtactgagagtgcaccatacaggaaacagc (SEQ ID |
| | NO: 108) |
| pTarget(AjTn6022)_ | cggggcggagatttcctggaagatgccaggaagatacttaacagggaagtgagagggccgc |
| U25D50 | ggcaaagccgtttttccataggctccgcccccctgacaagcatcacgaaatctgacgctcaaat |
| | cagtggtggcgaaacccgacaggactataaagataccaggcgtttccccctggcggctccctc |
| | gtgcgctctcctgttcctgcctttcggtttaccggtgtcattccgctgttatggccgcgtttgtctcat |
| | tccacgcctgacactcagttccgggtaggcagttcgctccaagctggactgtatgcacgaaccc |
| | cccgttcagtccgaccgctgcgccttatccggtaactatcgtcttgagtccaacccggaaagac |
| | atgcaaaagcaccactggcagcagccactggtaattgatttagaggagttagtcttgaagtcatg |
| | cgccggttaaggctaaactgaaaggacaagttttggtgactgcgctcctccaagccagttacct |
| | cggttcaaagagttggtagctcagagaaccttcgaaaaaccgccctgcaaggcggttttttcgtt |
| | ttcagagcaagagattacgcgcagaccaaaacgatctcaagaagatcatcttattaatatacgta |
| | gtatctacgatacgtagtatctacgatacgtagatcatggagaagtcatttaataaggccactgtta |
| | aacgtagatatatctacgcgtagatatatctacgatacgtagcggccgacgcgctgggctacgt |
| | ctttaatgggcagcgcccacatacgcagcgatttccaacagcttgaggcggaagctttaatgcg |
| | gtagtttatcacagttaaattgctaacgcagtcaggcaccgtgtatgaaatctaacaatgcgctca |
| | tcgtcatcctcggcaccgtcaccctggatgctgtaggcataggcttggttatgccggtactgccg |
| | ggcctcttgcgggatatcgtccattccgacagcatcgccagtcactatggcgtgctgctagcgc |
| | tatatgcgttgatgcaatttctatgcgcacccgttctcggagcactgtccgaccgctttggccgcc |
| | gcccagtcctgctcgcttcgctacttggagccactatcgactacgcgatcatggcgaccacacc |
| | cgtcctgtggatcctctacgccggacgcatcgtggccggcatcaccggcgccacaggtgcgg |
| | ttgctggcgcctatatcgccgacatcaccgatggggaagatcgggcctgccaccatacccacg |
| | ccgaaacaagcgccctgcaccattatgttccggatctgcatcgcaggatgctgctggctaccct |
| | gtggaacacctacatctgtattaacgaagcgctaaccgtttttatcaggctctgggaggcagaat |
| | aaatgatcatatcgtcaattattacctccacggggagagcctgagcaaactggcctcaggcattt |
| | gagaagcacacggtcacactgcttccggtagtcaataaaccggtaaaccagcaatagacataa |
| | gcggctatttaacgaccctgccctgaaccgacgaccgggtcgaatttgctttcgaatttctgccat |
| | tcatccgcttattatcacttattcaggcgtagcaaccaggcgtttaagggcaccaataactgcctt |
| | aaaaaaattacgccccgccctgccactcatcgcagtactgttgtaattcattaagcattctgccga |
| | catggaagccatcacaaacggcatgatgaacctgaatcgccagcggcatcagcaccttgtcgc |
| | cttgcgtataatatttgcccatggtgaaaacgggggcgaagaagttgtccatattggccacgttta |
| | aatcaaaactggtgaaactcacccagggattggctgagacgaaaaacatattctcaataaaccc |
| | tttagggaaataggccaggttttcaccgtaacacgccacatcttgcgaatatatgtgtagaaactg |
| | ccggaaatcgtcgtggtattcactccagagcgatgaaaacgtttcagtttgctcatggaaaacgg |
| | tgtaacaagggtgaacactatcccatatcaccagctcaccgtctttcattgccatacggaattccg |
| | gatgagcattcatcaggcgggcaagaatgtgaataaaggccggataaaacttgtgcttatttttct |
| | ttacggtctttaaaaaggccgtaatatccagctgaacggtctggttataggtacattgagcaactg |
| | actgaaatgcctcaaaatgttctttacgatgccattgggatatatcaacggtggtatatccagtgat |
| | ttttttctccattttagcttccttagctcctgaaaatctcgataactcaaaaaatacgcccgttctgcg |
| | gttgccttagtcggtggcgggtcacaacccaaacccggtgaaattactttggcacataaaggcg |
| | tgctgtagtgatcttatttcattatggtgaaagttggaacctcttacgtgccgatcaacgtctcatttt |
| | cgccaaaagttggcccagggcttcccggtatcaacagggacaccaggatttatttattctgcga |
| | agtgatcttccgtcacaggtatttattcggcgcaaagtgcgtcgggtgatgctgccaacttactga |
| | tttagtgtatgatggtgtttttgaggtgctccagtggcttctgtttctatcagctgtccctcctgttca |
| | cgtactgacggggtggtgcgtaacggcaaaagcaccgccggacatcagcgctagcggagtgt |
| | atactggcttactatgttggcactgatgagggtgtcagtgaagtgcttcatgtggcaggagaaaa |
| | aaggctgcaccggtgcgtcagcagaatatgtgatacaggatatattccgcttcctcgctcactga |
| | ctcgctacgctcggtcgttcgactgcggcgagcggaaatggcttacgaa (SEQ ID NO: |
| | 109) |
| pTarget(AjTn6022)_ | cggggcggagatttcctggaagatgccaggaagatacttaacagggaagtgagagggccgc |
| U50D25 | ggcaaagccgtttttccataggctccgcccccctgacaagcatcacgaaatctgacgctcaaat |
| | cagtggtggcgaaacccgacaggactataaagataccaggcgtttccccctggcggctccctc |
| | gtgcgctctcctgttcctgcctttcggtttaccggtgtcattccgctgttatggccgcgtttgtctcat |
| | tccacgcctgacactcagttccgggtaggcagttcgctccaagctggactgtatgcacgaaccc |
| | cccgttcagtccgaccgctgcgccttatccggtaactatcgtcttgagtccaacccggaaagac |
| | atgcaaaagcaccactggcagcagccactggtaattgatttagaggagttagtcttgaagtcatg |
| | cgccggttaaggctaaactgaaaggacaagttttggtgactgcgctcctccaagccagttacct |
| | cggttcaaagagttggtagctcagagaaccttcgaaaaaccgccctgcaaggcggttttttcgtt |
| | ttcagagcaagagattacgcgcagaccaaaacgatctcaagaagatcatcttattaatatacgta |
| | gtatctacgatacgtagtatctacgatacgtagatcatggagaagtcatttaataaggccactgtta |
| | aacgtagatatatctacgcgtagatatatctacgatacgtagcggccgacgcgctgggctacgt |
| | ctttaatgggcagcgcccacatacgcagcgatttccaacagcttgaggcggaagctttaatgcg |
| | gtagtttatcacagttaaattgctaacgcagtcaggcaccgtgtatgaaatctaacaatgcgctca |
| | tcgtcatcctcggcaccgtcaccctggatgctgtaggcataggcttggttatgccggtactgccg |
| | ggcctcttgcgggatatcgtccattccgacagcatcgccagtcactatggcgtgctgctagcgc |
| | tatatgcgttgatgcaatttctatgcgcacccgttctcggagcactgtccgaccgctttggccgcc |
| | gcccagtcctgctcgcttcgctacttggagccactatcgactacgcgatcatggcgaccacacc |
| | cgtcctgtggatcctctacgccggacgcatcgtggccggcatcaccggcgccacaggtgcgg |
| | ttgctggcgcctatatcgccgacatcaccgatggggaagatcgggcctgccaccatacccacg |
| | ccgaaacaagcgccctgcaccattatgttccggatctgcatcgcaggatgctgctggctaccct |
| | gtggaacacctacatctgtattaacgaagcgctaaccgtttttatcaggctctgggaggcagaat |
| | aaatgatcatatcgtcaattattacctccacggggagagcctgagcaaactggcctcaggcattt |
| | gagaagcacacggtcacactgcttccggtagtcaataaaccggtaaaccagcaatagacataa |
| | gcggctatttaacgaccctgccctgaaccgacgaccgggtcgaatttgctttcgaatttctgccat |
| | tcatccgcttattatcacttattcaggcgtagcaaccaggcgtttaagggcaccaataactgcctt |
| | aaaaaaattacgccccgccctgccactcatcgcagtactgttgtaattcattaagcattctgccga |
| | catggaagccatcacaaacggcatgatgaacctgaatcgccagcggcatcagcaccttgtcgc |
| | cttgcgtataatatttgcccatggtgaaaacgggggcgaagaagttgtccatattggccacgttta |
| | aatcaaaactggtgaaactcacccagggattggctgagacgaaaaacatattctcaataaaccc |
| | tttagggaaataggccaggttttcaccgtaacacgccacatcttgcgaatatatgtgtagaaactg |
| | ccggaaatcgtcgtggtattcactccagagcgatgaaaacgtttcagtttgctcatggaaaacgg |
| | tgtaacaagggtgaacactatcccatatcaccagctcaccgtctttcattgccatacggaattccg |
| | gatgagcattcatcaggcgggcaagaatgtgaataaaggccggataaaacttgtgcttatttttct |
| | ttacggtctttaaaaaggccgtaatatccagctgaacggtctggttataggtacattgagcaactg |
| | actgaaatgcctcaaaatgttctttacgatgccattgggatatatcaacggtggtatatccagtgat |
| | ttttttctccattttagcttccttagctcctgaaaatctcgataactcaaaaaatacgcccgcatttcg |
| | tgcaccgcaccatacagcttctgcggttgccttagtcggtggcgggtcacaacccaaacccggt |
| | gaaagtagtgatcttatttcattatggtgaaagttggaacctcttacgtgccgatcaacgtctcattt |
| | tcgccaaaagttggcccagggcttcccggtatcaacagggacaccaggatttatttattctgcga |
| | agtgatcttccgtcacaggtatttattcggcgcaaagtgcgtcgggtgatgctgccaacttactga |
| | tttagtgtatgatggtgtttttgaggtgctccagtggcttctgtttctatcagctgtccctcctgttca |
| | gctactgacggggtggtgcgtaacggcaaaagcaccgccggacatcagcgctagcggagtgt |
| | atactggcttactatgttggcactgatgagggtgtcagtgaagtgcttcatgtggcaggagaaaa |
| | aaggctgcaccggtgcgtcagcagaatatgtgatacaggatatattccgcttcctcgctcactga |
| | ctcgctacgctcggtcgttcgactgcggcgagcggaaatggcttacgaa (SEQ ID NO: |
| | 110) |
| pHelper(ZooTsy) | tttacactttatgcttccggctcgtatgttggtcgacgtggagatataccatgtttaactttaataagg |
| | agatatacatggcgaatccgaagatagtgctggcgacatccgatgtcgcttcgcgcgctctcgc |
| | gccagcagatgccgaactgttcgggaccatcattgatgcatggactcgtcgccgcatcggctc |
| | gctgcgccatacccacaaagccgtagccagggacgtctcggtgctgcgggagtttgctgccc |
| | actcaggtctgccgccttggctctggaatgaggatgcatttgactgctggtgcgatcatctcgga |
| | ttggagcgccggttggccaatagcacccagcggcattaccaatgctcgatccgatccttcctcg |
| | aatatctcacgacgaacgtcaagttcaggaacgagatccggcgcttgtatggcgtcgagccac |
| | gacagatttgcacggaagagaactgcatcccccataccgtcgaaaaggagtcctccaaagag |
| | cgccgagccttcacacatgaggaaatcgagttgttttttgacgggctgcggcgcgcttctgtcga |
| | ggccggccgcttcaggaccaaggattacaaccctcttcgtcgggaccgtactttctttttcaccct |
| | ctactccggagggctcaggatttccgaaggcctcaacctaaacgtaaattcgtttgcgccgaatc |
| | cagctttacctgaacttggcccctatggattcatcaaggtctggggcaagggaagccggggaa |
| | gtgggaagcgctttcggaccgtaccggtcacccatcctgacttccctgagctcatggactggta |
| | catcacaagagtccgccctgaattccttcatcgagcggaccccaatgaggaagccttgttcctct |
| | cggagcggggaactcgtctcaagtcgtccagcgccgacgcacgcttcaaaatcgcaactcgc |
| | tattgcggcctggaaggcgaaggactgacgccacactgtctgcgccactcgagcgttacccac |
| | gaatccttgcgcttcagcatcgagatggttcgtcggaagcatggccacgcttctgcggcaacga |
| | cacaaggctatatgcacgtccccgacgttatggtcgacgacgaaatcagcagcgtcgtctccc |
| | gcgagctcgatgacgcgctcaacacacaggagaagaaagaatgagcgatacgcccaatatc |
| | agacttggttggcgcctccgcgtgcggatggcggaaaaccgaataccgaccgccacggaact |
| | caagcgacggctcgacgcgatcggctacgacatcaccagcgcccaactctcccgaattgtgg |
| | acgagcgaccggcccaggtgaaaaccgcgctactggaggctttgcttaaagtcctgggggga |
| | accctagacgatctcatgcccgcggagggccgtgttgagcagccgagttctgcgcctcaaggt |
| | ggtgaggaagttgcgccaagcccagcgccggcggccacccccacccgtaagcgcaaaccg |
| | gcgcgccccgcctcggaagcagtcgatccggacgatgccggtggccccagggtacgcccct |
| | tctcggtcccaccgaagaagtgaaatagcgttcctacatggcgacctctgctcatacctgttgcg |
| | atgaatgcagccgtccgatggccaaggcgcgtctcgtccggaacgggaaggccttgtgcgac |
| | acctgcagccagcggattccctacgccccttgtggcaagtgcggaaagcagacgcgcatgct |
| | caatgaccaaggggaaccgctctgcaaggtctgccgtgccctcaaccgcacctgcgtgcgttg |
| | cggcaagccagtgcgcaaggctgggcgcttggtcgatggcggtgctgtctgcggcccctgcg |
| | ccaagtattaccgtgaaccgcaggcctgcgatcaatgtggggcaatggagaaggttcttaccg |
| | gcgtacagacagaaactgggcggcgccgtctctgcggcaggtgtttccggcgccataaaggc |
| | tacaagagctgcagcatctgccggaaagacaggctgcctgtcggcgtgaccgctgacggcaa |
| | gcctatatgcaaaacctgtttgagtgcggccggaaaacctttcgtgtgcccaacctgtggccag |
| | gaaggaatacgccatagtagaaggcgttgccagaagtgctacgttcgcgatacggcgaaaag |
| | ccgtgtcgaggaatatgctcccctttttagtcatgcatggacccgggaagcctggctgggatttg |
| | gtgcagagaccctggaggcaatgaatccagaaaagctagtgcatcgaatcaacagctactacc |
| | tcttcttcgtccggcttgatctcgccttcagcaaacctacagaaatcacgccggtcgcgctgttga |
| | aaagcgtcggcggcttggatgggctccgtcgatacaccgtgccctatggctatctcctgaagca |
| | ccgaatcatccccgagacaacgcgcagtctcctgcaggacgaggctgaatccatcaggcagg |
| | aagccatgatcgccaagctggatggcaagtggtatgccccagtcatggcacgctaccggaatc |
| | acctgctgactgtccgcgcccgatacgacagccggggctggaaaggggaagatacccggat |
| | gaaggcgcgctcgattacgctcaacctccgggcggctcggcgtttatgcgaaacgctggatgc |
| | cataggtgtccagatgattcagcagacgacgccagagatgctggacaagttcttcatggactat |
| | cctggactggccgtctcaatccgtgccttcgtccgatacctgaaccggaaggaaaaactctttca |
| | gcgcctgcgcctgaagacagttgcccggagtgtgccggaagggattttcctgccccgcgaaa |
| | aatccactgcgctcattcaagcgtggatgacgccgactgatgagacgcttcggcagtccctcat |
| | cggcctgttcatgctgctctatgcccagacggtcaagaactgtgttcggcttcgtctggatgcact |
| | catccaacgggctgacgggcgttatcgcatagcatttggccgcgcggaaatccacctggatgc |
| | tcggatcagcgcgatactcgatcgctatttgaacgtgcggcaaacactggttgctctggaccat |
| | gccgacgaaaatccgtatctctttccgggacggcagttaggcagtcacctgagtgccgaatcg |
| | gtatccggttggttgaaaaaggcgggcgtcagctccgatcagttgtttgcaacatcaatctataa |
| | cgcctatcaaaatggcctgcgacttcccaaggtcttggtgcgagcgtttggcatcaccgatacg |
| | accgcgatcaagtatctcaacatgctggatccccgcctggtcatggagatcgaacgcaaacca |
| | ctgggcggcggggacgaaccctgatggctggcgaacttaccgttcggaaaaatccatacgcc |
| | agcggtcagccgtggtggctctatatgctcgaatgcaagggcggaggcatctacaccggcatc |
| | gcgctggatgtggatgcgcggtttgagcaacacgtcgcaggcaagggcgccaaatacacgc |
| | gcatgaaccggcccatccgggtcgtcgcgcgcatccgctacgacacgcaccgggaagccgc |
| | tcaggctgaagttgcgctgaaacgcctcaagcccctggaaaagctgcggtgggcctatgcctt |
| | ggctgggttagtcggcatggacaagtcggcgtaacgcgtggggcgtctccggatcgttgagg |
| | ggcaaaagcatcgcggcccccgaggctgctgccaccgctgagcaataacctaggctgctgcc |
| | accgctgagcaataactagcataaccccttggggcctctaaacgggtcttgaggggttttttgct |
| | gaaacctcaggcatttgagaagcacacggtcacactgcttccggtagtcaataaaccggtaaac |
| | cagcaatagacataagcggctatttaacgaccctgccctgaaccgacgaccgggtcatcgtgg |
| | ccggatcttgcggcccctcggcttgaacgaattgttagacattatttgccgactaccttggtgatct |
| | cgcctttcacgtagtggacaaattcttccaactgatctgcgcgcgaggccaagcgatcttcttctt |
| | gtccaagataagcctgtctagcttcaagtatgacgggctgatactgggccggcaggcgctccat |
| | tgcccagtcggcagcgacatccttcggcgcgattttgccggttactgcgctgtaccaaatgcgg |
| | gacaacgtaagcactacatttcgctcatcgccagcccagtcgggcggcgagttccatagcgtta |
| | aggtttcatttagcgcctcaaatagatcctgttcaggaaccggatcaaagagttcctccgccgct |
| | ggacctaccaaggcaacgctatgttctcttgcttttgtcagcaagatagccagatcaatgtcgatc |
| | gtggctggctcgaagatacctgcaagaatgtcattgcgctgccattctccaaattgcagttcgcg |
| | cttagctggataacgccacggaatgatgtcgtcgtgcacaacaatggtgacttctacagcgcgg |
| | agaatctcgctctctccaggggaagccgaagtttccaaaaggtcgttgatcaaagctcgccgcg |
| | ttgtttcatcaagccttacggtcaccgtaaccagcaaatcaatatcactgtgtggcttcaggccgc |
| | catccactgcggagccgtacaaatgtacggccagcaacgtcggttcgagatggcgctcgatga |
| | cgccaactacctctgatagttgagtcgatacttcggcgatcaccgcttccctcatactcttccttttt |
| | caatattattgaagcatttatcagggttattgtctcatgagcggatacatatttgaatgtatttagaaa |
| | aataaacaaatagctagctcactcggtcgctacgctccgggcgtgagactgcggcgggcgct |
| | gcggacacatacaaagttacccacagattccgtggataagcaggggactaacatgtgaggca |
| | aaacagcagggccgcgccggtggcgtttttccataggctccgccctcctgccagagttcacata |
| | aacagacgcttttccggtgcatctgtgggagccgtgaggctcaaccatgaatctgacagtacgg |
| | gcgaaacccgacaggacttaaagatccccaccgtttccggcgggtcgctccctcttgcgctctc |
| | ctgttccgaccctgccgtttaccggatacctgttccgcctttctcccttacgggaagtgtggcgctt |
| | tctcatagctcacacactggtatctcggctcggtgtaggtcgttcgctccaagctgggctgtaag |
| | caagaactccccgttcagcccgactgctgcgccttatccggtaactgttcacttgagtccaaccc |
| | ggaaaagcacggtaaaacgccactggcagcagccattggtaactgggagttcgcagaggatt |
| | tgtttagctaaacacgcggttgctcttgaagtgtgcgccaaagtccggctacactggaaggaca |
| | gatttggttgctgtgctctgcgaaagccagttaccacggttaagcagttccccaactgacttaacc |
| | ttcgatcaaaccacctccccaggggttttttcgtttacagggcaaaagattacgcgcagaaaaa |
| | aaggatctcaagaagatcctttgatcttttctactgaaccgctctagatttcagtgcaatttatctctt |
| | caaatgtagcacctgaagtcagccccatacgatataagttgtaattctcatgttagtcatgccccg |
| | cgcccaccggaaggagctgactgggttgaaggctctcaagggcatcggtcgagatcccggtg |
| | cctaatgagtgagctaacttacattaattgcgttgcgc (SEQ ID NO: 111) |
| pHelper(ZooTsy)_ | tttacactttatgcttccggctcgtatgttggtcgacgtggagatataccatgtttaactttaataagg |
| ΔYRec | agatatacatgagcgatacgcccaatatcagacttggttggcgcctccgcgtgcggatggcgg |
| | aaaaccgaataccgaccgccacggaactcaagcgacggctcgacgcgatcggctacgacat |
| | caccagcgcccaactctcccgaattgtggacgagcgaccggcccaggtgaaaaccgcgcta |
| | ctggaggctttgcttaaagtcctggggggaaccctagacgatctcatgcccgcggagggccgt |
| | gttgagcagccgagttctgcgcctcaaggtggtgaggaagttgcgccaagcccagcgccggc |
| | ggccacccccacccgtaagcgcaaaccggcgcgccccgcctcggaagcagtcgatccgga |
| | cgatgccggtggccccagggtacgccccttctcggtcccaccgaagaagtgaaatagcgttcc |
| | tacatggcgacctctgctcatacctgttgcgatgaatgcagccgtccgatggccaaggcgcgtc |
| | tcgtccggaacgggaaggccttgtgcgacacctgcagccagcggattccctacgccccttgtg |
| | gcaagtgcggaaagcagacgcgcatgctcaatgaccaaggggaaccgctctgcaaggtctg |
| | ccgtgccctcaaccgcacctgcgtgcgttgcggcaagccagtgcgcaaggctgggcgcttgg |
| | tcgatggcggtgctgtctgcggcccctgcgccaagtattaccgtgaaccgcaggcctgcgatc |
| | aatgtggggcaatggagaaggttcttaccggcgtacagacagaaactgggggcgccgtctc |
| | tgcggcaggtgtttccggcgccataaaggctacaagagctgcagcatctgccggaaagacag |
| | gctgcctgtcggcgtgaccgctgacggcaagcctatatgcaaaacctgtttgagtgcggccgg |
| | aaaacctttcgtgtgcccaacctgtggccaggaaggaatacgccatagtagaaggcgttgcca |
| | gaagtgctacgttcgcgatacggcgaaaagccgtgtcgaggaatatgctcccctttttagtcatg |
| | catggacccgggaagcctggctgggatttggtgcagagaccctggaggcaatgaatccagaa |
| | aagctagtgcatcgaatcaacagctactacctcttcttcgtccggcttgatctcgccttcagcaaa |
| | cctacagaaatcacgccggtcgcgctgttgaaaagcgtcggcggcttggatgggctccgtcga |
| | tacaccgtgccctatggctatctcctgaagcaccgaatcatccccgagacaacgcgcagtctcc |
| | tgcaggacgaggctgaatccatcaggcaggaagccatgatcgccaagctggatggcaagtg |
| | gtatgccccagtcatggcacgctaccggaatcacctgctgactgtccgcgcccgatacgacag |
| | ccggggctggaaaggggaagatacccggatgaaggcgcgctcgattacgctcaacctccgg |
| | gcggctcggcgtttatgcgaaacgctggatgccataggtgtccagatgattcagcagacgacg |
| | ccagagatgctggacaagttcttcatggactatcctggactggccgtctcaatccgtgccttcgtc |
| | cgatacctgaaccggaaggaaaaactctttcagcgcctgcgcctgaagacagttgcccggagt |
| | gtgccggaagggattttcctgccccgcgaaaaatccactgcgctcattcaagcgtggatgacg |
| | ccgactgatgagacgcttcggcagtccctcatcggcctgttcatgctgctctatgcccagacggt |
| | caagaactgtgttcggcttcgtctggatgcactcatccaacgggctgacgggcgttatcgcata |
| | gcatttggccgcgcggaaatccacctggatgctcggatcagcgcgatactcgatcgctatttga |
| | acgtgcggcaaacactggttgctctggaccatgccgacgaaaatccgtatctctttccgggacg |
| | gcagttaggcagtcacctgagtgccgaatcggtatccggttggttgaaaaaggcgggcgtcag |
| | ctccgatcagttgtttgcaacatcaatctataacgcctatcaaaatggcctgcgacttcccaaggt |
| | cttggtgcgagcgtttggcatcaccgatacgaccgcgatcaagtatctcaacatgctggatccc |
| | cgcctggtcatggagatcgaacgcaaaccactgggcggcggggacgaaccctgatggctgg |
| | cgaacttaccgttcggaaaaatccatacgccagcggtcagccgtggtggctctatatgctcgaa |
| | tgcaagggcggaggcatctacaccggcatcgcgctggatgtggatgcgcggtttgagcaaca |
| | cgtcgcaggcaagggcgccaaatacacgcgcatgaaccggcccatccgggtcgtcgcgcgc |
| | atccgctacgacacgcaccgggaagccgctcaggctgaagttgcgctgaaacgcctcaagcc |
| | cctggaaaagctgcggtgggcctatgccttggctgggttagtcggcatggacaagtcggcgta |
| | acgcgtggggcgtctccggatcgttgaggggcaaaagcatcgcggcccccgaggctgctgc |
| | caccgctgagcaataacctaggctgctgccaccgctgagcaataactagcataaccccttggg |
| | gcctctaaacgggtcttgaggggttttttgctgaaacctcaggcatttgagaagcacacggtcac |
| | actgcttccggtagtcaataaaccggtaaaccagcaatagacataagcggctatttaacgaccct |
| | gccctgaaccgacgaccgggtcatcgtggccggatcttgcggcccctcggcttgaacgaattg |
| | ttagacattatttgccgactaccttggtgatctcgcctttcacgtagtggacaaattcttccaactga |
| | tctgcgcgcgaggccaagcgatcttcttcttgtccaagataagcctgtctagcttcaagtatgacg |
| | ggctgatactgggccggcaggcgctccattgcccagtcggcagcgacatccttcggcgcgatt |
| | ttgccggttactgcgctgtaccaaatgcgggacaacgtaagcactacatttcgctcatcgccagc |
| | ccagtcgggcggcgagttccatagcgttaaggtttcatttagcgcctcaaatagatcctgttcag |
| | gaaccggatcaaagagttcctccgccgctggacctaccaaggcaacgctatgttctcttgctttt |
| | gtcagcaagatagccagatcaatgtcgatcgtggctggctcgaagatacctgcaagaatgtcat |
| | tgcgctgccattctccaaattgcagttcgcgcttagctggataacgccacggaatgatgtcgtcg |
| | tgcacaacaatggtgacttctacagcgcggagaatctcgctctctccaggggaagccgaagttt |
| | ccaaaaggtcgttgatcaaagctcgccgcgttgtttcatcaagccttacggtcaccgtaaccagc |
| | aaatcaatatcactgtgtggcttcaggccgccatccactgcggagccgtacaaatgtacggcca |
| | gcaacgtcggttcgagatggcgctcgatgacgccaactacctctgatagttgagtcgatacttc |
| | ggcgatcaccgcttccctcatactcttcctttttcaatattattgaagcatttatcagggttattgtct |
| | catgagcggatacatatttgaatgtatttagaaaaataaacaaatagctagctcactcggtcgctac |
| | gctccgggcgtgagactgcggcgggcgctgcggacacatacaaagttacccacagattccgt |
| | ggataagcaggggactaacatgtgaggcaaaacagcagggccgcgccggtggcgtttttcca |
| | taggctccgccctcctgccagagttcacataaacagacgcttttccggtgcatctgtgggagcc |
| | gtgaggctcaaccatgaatctgacagtacgggcgaaacccgacaggacttaaagatccccac |
| | cgtttccggcgggtcgctccctcttgcgctctcctgttccgaccctgccgtttaccggatacctgtt |
| | ccgcctttctcccttacgggaagtgtggcgctttctcatagctcacacactggtatctcggctcgg |
| | tgtaggtcgttcgctccaagctgggctgtaagcaagaactccccgttcagcccgactgctgcgc |
| | cttatccggtaactgttcacttgagtccaacccggaaaagcacggtaaaacgccactggcagca |
| | gccattggtaactgggagttcgcagaggatttgtttagctaaacacgcggttgctcttgaagtgtg |
| | cgccaaagtccggctacactggaaggacagatttggttgctgtgctctgcgaaagccagttacc |
| | acggttaagcagttccccaactgacttaaccttcgatcaaaccacctccccaggggttttttcgtt |
| | tacagggcaaaagattacgcgcagaaaaaaaggatctcaagaagatcctttgatcttttctactg |
| | aaccgctctagatttcagtgcaatttatctcttcaaatgtagcacctgaagtcagccccatacgata |
| | taagttgtaattctcatgttagtcatgccccgcgcccaccggaaggagctgactgggttgaagg |
| | ctctcaagggcatcggtcgagatcccggtgcctaatgagtgagctaacttacattaattgcgttg |
| | cgc (SEQ ID NO: 112) |
| pHelper(ZooTsy)_ | tttacactttatgcttccggctcgtatgttggtcgacgtggagatataccatgtttaactttaataagg |
| ΔHTH | agatatacatggcgaatccgaagatagtgctggcgacatccgatgtcgcttcgcgcgctctcgc |
| | gccagcagatgccgaactgttcgggaccatcattgatgcatggactcgtcgccgcatcggctc |
| | gctgcgccatacccacaaagccgtagccagggacgtctcggtgctgcgggagtttgctgccc |
| | actcaggtctgccgccttggctctggaatgaggatgcatttgactgctggtgcgatcatctcgga |
| | ttggagcgccggttggccaatagcacccagcggcattaccaatgctcgatccgatccttcctcg |
| | aatatctcacgacgaacgtcaagttcaggaacgagatccggcgcttgtatggcgtcgagccac |
| | gacagatttgcacggaagagaactgcatcccccataccgtcgaaaaggagtcctccaaagag |
| | cgccgagccttcacacatgaggaaatcgagttgttttttgacgggctgcggcgcgcttctgtcga |
| | ggccggccgcttcaggaccaaggattacaaccctcttcgtcgggaccgtactttctttttcaccct |
| | ctactccggagggctcaggatttccgaaggcctcaacctaaacgtaaattcgtttgcgccgaatc |
| | cagctttacctgaacttggcccctatggattcatcaaggtctggggcaagggaagccggggaa |
| | gtgggaagcgctttcggaccgtaccggtcacccatcctgacttccctgagctcatggactggta |
| | catcacaagagtccgccctgaattccttcatcgagcggaccccaatgaggaagccttgttcctct |
| | cggagcggggaactcgtctcaagtcgtccagcgccgacgcacgcttcaaaatcgcaactcgc |
| | tattgcggcctggaaggcgaaggactgacgccacactgtctgcgccactcgagcgttacccac |
| | gaatccttgcgcttcagcatcgagatggttcgtcggaagcatggccacgcttctgcggcaacga |
| | cacaaggctatatgcacgtccccgacgttatggtcgacgacgaaatcagcagcgtcgtctccc |
| | gcgagctcgatgacgcgctcaacacacaggagaagaaagaatgaaatagcgttcctacatgg |
| | cgacctctgctcatacctgttgcgatgaatgcagccgtccgatggccaaggcgcgtctcgtccg |
| | gaacgggaaggccttgtgcgacacctgcagccagcggattccctacgccccttgtggcaagt |
| | gcggaaagcagacgcgcatgctcaatgaccaaggggaaccgctctgcaaggtctgccgtgc |
| | cctcaaccgcacctgcgtgcgttgcggcaagccagtgcgcaaggctgggcgcttggtcgatg |
| | gcggtgctgtctgcggcccctgcgccaagtattaccgtgaaccgcaggcctgcgatcaatgtg |
| | gggcaatggagaaggttcttaccggcgtacagacagaaactgggcggcgccgtctctgcggc |
| | aggtgtttccggcgccataaaggctacaagagctgcagcatctgccggaaagacaggctgcct |
| | gtcggcgtgaccgctgacggcaagcctatatgcaaaacctgtttgagtgcggccggaaaacct |
| | ttcgtgtgcccaacctgtggccaggaaggaatacgccatagtagaaggcgttgccagaagtgc |
| | tacgttcgcgatacggcgaaaagccgtgtcgaggaatatgctcccctttttagtcatgcatggac |
| | ccgggaagcctggctgggatttggtgcagagaccctggaggcaatgaatccagaaaagctag |
| | tgcatcgaatcaacagctactacctcttcttcgtccggcttgatctcgccttcagcaaacctacag |
| | aaatcacgccggtcgcgctgttgaaaagcgtcggcggcttggatgggctccgtcgatacaccg |
| | tgccctatggctatctcctgaagcaccgaatcatccccgagacaacgcgcagtctcctgcagga |
| | cgaggctgaatccatcaggcaggaagccatgatcgccaagctggatggcaagtggtatgccc |
| | cagtcatggcacgctaccggaatcacctgctgactgtccgcgcccgatacgacagccggggc |
| | tggaaaggggaagatacccggatgaaggcgcgctcgattacgctcaacctccgggcggctc |
| | ggcgtttatgcgaaacgctggatgccataggtgtccagatgattcagcagacgacgccagaga |
| | tgctggacaagttcttcatggactatcctggactggccgtctcaatccgtgccttcgtccgatacc |
| | tgaaccggaaggaaaaactctttcagcgcctgcgcctgaagacagttgcccggagtgtgccg |
| | gaagggattttcctgccccgcgaaaaatccactgcgctcattcaagcgtggatgacgccgactg |
| | atgagacgcttcggcagtccctcatcggcctgttcatgctgctctatgcccagacggtcaagaa |
| | ctgtgttcggcttcgtctggatgcactcatccaacgggctgacgggcgttatcgcatagcatttg |
| | gccgcgcggaaatccacctggatgctcggatcagcgcgatactcgatcgctatttgaacgtgc |
| | ggcaaacactggttgctctggaccatgccgacgaaaatccgtatctctttccgggacggcagtt |
| | aggcagtcacctgagtgccgaatcggtatccggttggttgaaaaaggcgggcgtcagctccg |
| | atcagttgtttgcaacatcaatctataacgcctatcaaaatggcctgcgacttcccaaggtcttggt |
| | gcgagcgtttggcatcaccgatacgaccgcgatcaagtatctcaacatgctggatccccgcctg |
| | gtcatggagatcgaacgcaaaccactgggcggcggggacgaaccctgatggctggcgaact |
| | taccgttcggaaaaatccatacgccagcggtcagccgtggtggctctatatgctcgaatgcaag |
| | ggcggaggcatctacaccggcatcgcgctggatgtggatgcgcggtttgagcaacacgtcgc |
| | aggcaagggcgccaaatacacgcgcatgaaccggcccatccgggtcgtcgcgcgcatccgc |
| | tacgacacgcaccgggaagccgctcaggctgaagttgcgctgaaacgcctcaagcccctgga |
| | aaagctgcggtgggcctatgccttggctgggttagtcggcatggacaagtcggcgtaacgcgt |
| | ggggcgtctccggatcgttgaggggcaaaagcatcgcggcccccgaggctgctgccaccgc |
| | tgagcaataacctaggctgctgccaccgctgagcaataactagcataaccccttggggcctcta |
| | aacgggtcttgaggggttttttgctgaaacctcaggcatttgagaagcacacggtcacactgctt |
| | ccggtagtcaataaaccggtaaaccagcaatagacataagcggctatttaacgaccctgccctg |
| | aaccgacgaccgggtcatcgtggccggatcttgcggcccctcggcttgaacgaattgttagac |
| | attatttgccgactaccttggtgatctcgcctttcacgtagtggacaaattcttccaactgatctgcg |
| | cgcgaggccaagcgatcttcttcttgtccaagataagcctgtctagcttcaagtatgacgggctg |
| | atactgggccggcaggcgctccattgcccagtcggcagcgacatccttcggcgcgattttgcc |
| | ggttactgcgctgtaccaaatgcgggacaacgtaagcactacatttcgctcatcgccagcccag |
| | tcgggcggcgagttccatagcgttaaggtttcatttagcgcctcaaatagatcctgttcaggaac |
| | cggatcaaagagttcctccgccgctggacctaccaaggcaacgctatgttctcttgcttttgtcag |
| | caagatagccagatcaatgtcgatcgtggctggctcgaagatacctgcaagaatgtcattgcgc |
| | tgccattctccaaattgcagttcgcgcttagctggataacgccacggaatgatgtcgtcgtgcac |
| | aacaatggtgacttctacagcgcggagaatctcgctctctccaggggaagccgaagtttccaaa |
| | aggtcgttgatcaaagctcgccgcgttgtttcatcaagccttacggtcaccgtaaccagcaaatc |
| | aatatcactgtgtggcttcaggccgccatccactgcggagccgtacaaatgtacggccagcaa |
| | cgtcggttcgagatggcgctcgatgacgccaactacctctgatagttgagtcgatacttcggcg |
| | atcaccgcttccctcatactcttcctttttcaatattattgaagcatttatcagggttattgtctcatg |
| | agcggatacatatttgaatgtatttagaaaaataaacaaatagctagctcactcggtcgctacgctcc |
| | gggcgtgagactgcggcgggcgctgcggacacatacaaagttacccacagattccgtggata |
| | agcaggggactaacatgtgaggcaaaacagcagggccgcgccggtggcgtttttccataggc |
| | tccgccctcctgccagagttcacataaacagacgcttttccggtgcatctgtgggagccgtgag |
| | gctcaaccatgaatctgacagtacgggcgaaacccgacaggacttaaagatccccaccgtttc |
| | cggcgggtcgctccctcttgcgctctcctgttccgaccctgccgtttaccggatacctgttccgcc |
| | tttctcccttacgggaagtgtggcgctttctcatagctcacacactggtatctcggctcggtgtag |
| | gtcgttcgctccaagctgggctgtaagcaagaactccccgttcagcccgactgctgcgccttat |
| | ccggtaactgttcacttgagtccaacccggaaaagcacggtaaaacgccactggcagcagcc |
| | attggtaactgggagttcgcagaggatttgtttagctaaacacgcggttgctcttgaagtgtgcgc |
| | caaagtccggctacactggaaggacagatttggttgctgtgctctgcgaaagccagttaccacg |
| | gttaagcagttccccaactgacttaaccttcgatcaaaccacctccccaggtggttttttcgtttac |
| | agggcaaaagattacgcgcagaaaaaaaggatctcaagaagatcctttgatcttttctactgaac |
| | cgctctagatttcagtgcaatttatctcttcaaatgtagcacctgaagtcagccccatacgatataa |
| | gttgtaattctcatgttagtcatgccccgcgcccaccggaaggagctgactgggttgaaggctct |
| | caagggcatcggtcgagatcccggtgcctaatgagtgagctaacttacattaattgcgttgcgc |
| | (SEQ ID NO: 113) |
| pHelper(ZooTsy)_ | tttacactttatgcttccggctcgtatgttggtcgacgtggagatataccatgtttaactttaataagg |
| ΔTnsF | agatatacatggcgaatccgaagatagtgctggcgacatccgatgtcgcttcgcgcgctctcgc |
| | gccagcagatgccgaactgttcgggaccatcattgatgcatggactcgtcgccgcatcggctc |
| | gctgcgccatacccacaaagccgtagccagggacgtctcggtgctgcgggagtttgctgccc |
| | actcaggtctgccgccttggctctggaatgaggatgcatttgactgctggtgcgatcatctcgga |
| | ttggagcgccggttggccaatagcacccagcggcattaccaatgctcgatccgatccttcctcg |
| | aatatctcacgacgaacgtcaagttcaggaacgagatccggcgcttgtatggcgtcgagccac |
| | gacagatttgcacggaagagaactgcatcccccataccgtcgaaaaggagtcctccaaagag |
| | cgccgagccttcacacatgaggaaatcgagttgttttttgacgggctgcggcgcgcttctgtcga |
| | ggccggccgcttcaggaccaaggattacaaccctcttcgtcgggaccgtactttctttttcaccct |
| | ctactccggagggctcaggatttccgaaggcctcaacctaaacgtaaattcgtttgcgccgaatc |
| | cagctttacctgaacttggcccctatggattcatcaaggtctggggcaagggaagccggggaa |
| | gtgggaagcgctttcggaccgtaccggtcacccatcctgacttccctgagctcatggactggta |
| | catcacaagagtccgccctgaattccttcatcgagcggaccccaatgaggaagccttgttcctct |
| | cggagcggggaactcgtctcaagtcgtccagcgccgacgcacgcttcaaaatcgcaactcgc |
| | tattgcggcctggaaggcgaaggactgacgccacactgtctgcgccactcgagcgttacccac |
| | gaatccttgcgcttcagcatcgagatggttcgtcggaagcatggccacgcttctgcggcaacga |
| | cacaaggctatatgcacgtccccgacgttatggtcgacgacgaaatcagcagcgtcgtctccc |
| | gcgagctcgatgacgcgctcaacacacaggagaagaaagaatgagcgatacgcccaatatc |
| | agacttggttggcgcctccgcgtgcggatggcggaaaaccgaataccgaccgccacggaact |
| | caagcgacggctcgacgcgatcggctacgacatcaccagcgcccaactctcccgaattgtgg |
| | acgagcgaccggcccaggtgaaaaccgcgctactggaggctttgcttaaagtcctgggggga |
| | accctagacgatctcatgcccgcggagggccgtgttgagcagccgagttctgcgcctcaaggt |
| | ggtgaggaagttgcgccaagcccagcgccggcggccacccccacccgtaagcgcaaaccg |
| | gcgcgccccgcctcggaagcagtcgatccggacgatgccggtggccccagggtacgcccct |
| | tctcggtcccaccgaagaagtgaaatagcgttcctacatggctggcgaacttaccgttcggaaa |
| | aatccatacgccagcggtcagccgtggtggctctatatgctcgaatgcaagggcggaggcatc |
| | tacaccggcatcgcgctggatgtggatgcgcggtttgagcaacacgtcgcaggcaagggcgc |
| | caaatacacgcgcatgaaccggcccatccgggtcgtcgcgcgcatccgctacgacacgcacc |
| | gggaagccgctcaggctgaagttgcgctgaaacgcctcaagcccctggaaaagctgcggtg |
| | ggcctatgccttggctgggttagtcggcatggacaagtcggcgtaacgcgtggggcgtctccg |
| | gatcgttgaggggcaaaagcatcgcggcccccgaggctgctgccaccgctgagcaataacct |
| | aggctgctgccaccgctgagcaataactagcataaccccttggggcctctaaacgggtcttgag |
| | gggttttttgctgaaacctcaggcatttgagaagcacacggtcacactgcttccggtagtcaataa |
| | accggtaaaccagcaatagacataagcggctatttaacgaccctgccctgaaccgacgaccg |
| | ggtcatcgtggccggatcttgcggcccctcggcttgaacgaattgttagacattatttgccgacta |
| | ccttggtgatctcgcctttcacgtagtggacaaattcttccaactgatctgcgcgcgaggccaag |
| | cgatcttcttcttgtccaagataagcctgtctagcttcaagtatgacgggctgatactgggccggc |
| | aggcgctccattgcccagtcggcagcgacatccttcggcgcgattttgccggttactgcgctgt |
| | accaaatgcgggacaacgtaagcactacatttcgctcatcgccagcccagtcgggcggcgag |
| | ttccatagcgttaaggtttcatttagcgcctcaaatagatcctgttcaggaaccggatcaaagagtt |
| | cctccgccgctggacctaccaaggcaacgctatgttctcttgcttttgtcagcaagatagccaga |
| | tcaatgtcgatcgtggctggctcgaagatacctgcaagaatgtcattgcgctgccattctccaaat |
| | tgcagttcgcgcttagctggataacgccacggaatgatgtcgtcgtgcacaacaatggtgactt |
| | ctacagcgcggagaatctcgctctctccaggggaagccgaagtttccaaaaggtcgttgatcaa |
| | agctcgccgcgttgtttcatcaagccttacggtcaccgtaaccagcaaatcaatatcactgtgtg |
| | gcttcaggccgccatccactgcggagccgtacaaatgtacggccagcaacgtcggttcgagat |
| | ggcgctcgatgacgccaactacctctgatagttgagtcgatacttcggcgatcaccgcttccctc |
| | atactcttcctttttcaatattattgaagcatttatcagggttattgtctcatgagcggatacatattt |
| | gaatgtatttagaaaaataaacaaatagctagctcactcggtcgctacgctccgggcgtgagactgc |
| | gggggcgctgcggacacatacaaagttacccacagattccgtggataagcaggggactaac |
| | atgtgaggcaaaacagcagggccgcgccggtggcgtttttccataggctccgccctcctgcca |
| | gagttcacataaacagacgcttttccggtgcatctgtgggagccgtgaggctcaaccatgaatct |
| | gacagtacgggcgaaacccgacaggacttaaagatccccaccgtttccgggggtcgctccc |
| | tcttgcgctctcctgttccgaccctgccgtttaccggatacctgttccgcctttctcccttacggga |
| | agtgtggcgctttctcatagctcacacactggtatctcggctcggtgtaggtcgttcgctccaagc |
| | tgggctgtaagcaagaactccccgttcagcccgactgctgcgccttatccggtaactgttcactt |
| | gagtccaacccggaaaagcacggtaaaacgccactggcagcagccattggtaactgggagtt |
| | cgcagaggatttgtttagctaaacacgcggttgctcttgaagtgtgcgccaaagtccggctaca |
| | ctggaaggacagatttggttgctgtgctctgcgaaagccagttaccacggttaagcagttcccc |
| | aactgacttaaccttcgatcaaaccacctccccaggtggttttttcgtttacagggcaaaagattac |
| | gcgcagaaaaaaaggatctcaagaagatcctttgatcttttctactgaaccgctctagatttcagt |
| | gcaatttatctcttcaaatgtagcacctgaagtcagccccatacgatataagttgtaattctcatgtt |
| | agtcatgccccgcgcccaccggaaggagctgactgggttgaaggctctcaagggcatcggtc |
| | gagatcccggtgcctaatgagtgagctaacttacattaattgcgttgcgc (SEQ ID NO: |
| | 114) |
| pHelper(ZooTsy)_ | tttacactttatgcttccggctcgtatgttggtcgacgtggagatataccatgtttaactttaataagg |
| ΔGIY-YIG | agatatacatggcgaatccgaagatagtgctggcgacatccgatgtcgcttcgcgcgctctcgc |
| | gccagcagatgccgaactgttcgggaccatcattgatgcatggactcgtcgccgcatcggctc |
| | gctgcgccatacccacaaagccgtagccagggacgtctcggtgctgcgggagtttgctgccc |
| | actcaggtctgccgccttggctctggaatgaggatgcatttgactgctggtgcgatcatctcgga |
| | ttggagcgccggttggccaatagcacccagcggcattaccaatgctcgatccgatccttcctcg |
| | aatatctcacgacgaacgtcaagttcaggaacgagatccggcgcttgtatggcgtcgagccac |
| | gacagatttgcacggaagagaactgcatcccccataccgtcgaaaaggagtcctccaaagag |
| | cgccgagccttcacacatgaggaaatcgagttgttttttgacgggctgcggcgcgcttctgtcga |
| | ggccggccgcttcaggaccaaggattacaaccctcttcgtcgggaccgtactttctttttcaccct |
| | ctactccggagggctcaggatttccgaaggcctcaacctaaacgtaaattcgtttgcgccgaatc |
| | cagctttacctgaacttggcccctatggattcatcaaggtctggggcaagggaagccggggaa |
| | gtgggaagcgctttcggaccgtaccggtcacccatcctgacttccctgagctcatggactggta |
| | catcacaagagtccgccctgaattccttcatcgagcggaccccaatgaggaagccttgttcctct |
| | cggagcggggaactcgtctcaagtcgtccagcgccgacgcacgcttcaaaatcgcaactcgc |
| | tattgcggcctggaaggcgaaggactgacgccacactgtctgcgccactcgagcgttacccac |
| | gaatccttgcgcttcagcatcgagatggttcgtcggaagcatggccacgcttctgcggcaacga |
| | cacaaggctatatgcacgtccccgacgttatggtcgacgacgaaatcagcagcgtcgtctccc |
| | gcgagctcgatgacgcgctcaacacacaggagaagaaagaatgagcgatacgcccaatatc |
| | agacttggttggcgcctccgcgtgcggatggcggaaaaccgaataccgaccgccacggaact |
| | caagcgacggctcgacgcgatcggctacgacatcaccagcgcccaactctcccgaattgtgg |
| | acgagcgaccggcccaggtgaaaaccgcgctactggaggctttgcttaaagtcctgggggga |
| | accctagacgatctcatgcccgcggagggccgtgttgagcagccgagttctgcgcctcaaggt |
| | ggtgaggaagttgcgccaagcccagcgccggcggccacccccacccgtaagcgcaaaccg |
| | gcgcgccccgcctcggaagcagtcgatccggacgatgccggtggccccagggtacgcccct |
| | tctcggtcccaccgaagaagtgaaatagcgttcctacatggcgacctctgctcatacctgttgcg |
| | atgaatgcagccgtccgatggccaaggcgcgtctcgtccggaacgggaaggccttgtgcgac |
| | acctgcagccagcggattccctacgccccttgtggcaagtgcggaaagcagacgcgcatgct |
| | caatgaccaaggggaaccgctctgcaaggtctgccgtgccctcaaccgcacctgcgtgcgttg |
| | cggcaagccagtgcgcaaggctgggcgcttggtcgatggcggtgctgtctgcggcccctgcg |
| | ccaagtattaccgtgaaccgcaggcctgcgatcaatgtggggcaatggagaaggttcttaccg |
| | gcgtacagacagaaactgggcggcgccgtctctgcggcaggtgtttccggcgccataaaggc |
| | tacaagagctgcagcatctgccggaaagacaggctgcctgtcggcgtgaccgctgacggcaa |
| | gcctatatgcaaaacctgtttgagtgcggccggaaaacctttcgtgtgcccaacctgtggccag |
| | gaaggaatacgccatagtagaaggcgttgccagaagtgctacgttcgcgatacggcgaaaag |
| | ccgtgtcgaggaatatgctcccctttttagtcatgcatggacccgggaagcctggctgggatttg |
| | gtgcagagaccctggaggcaatgaatccagaaaagctagtgcatcgaatcaacagctactacc |
| | tcttcttcgtccggcttgatctcgccttcagcaaacctacagaaatcacgccggtcgcgctgttga |
| | aaagcgtcggcggcttggatgggctccgtcgatacaccgtgccctatggctatctcctgaagca |
| | ccgaatcatccccgagacaacgcgcagtctcctgcaggacgaggctgaatccatcaggcagg |
| | aagccatgatcgccaagctggatggcaagtggtatgccccagtcatggcacgctaccggaatc |
| | acctgctgactgtccgcgcccgatacgacagccggggctggaaaggggaagatacccggat |
| | gaaggcgcgctcgattacgctcaacctccgggcggctcggcgtttatgcgaaacgctggatgc |
| | cataggtgtccagatgattcagcagacgacgccagagatgctggacaagttcttcatggactat |
| | cctggactggccgtctcaatccgtgccttcgtccgatacctgaaccggaaggaaaaactctttca |
| | gcgcctgcgcctgaagacagttgcccggagtgtgccggaagggattttcctgccccgcgaaa |
| | aatccactgcgctcattcaagcgtggatgacgccgactgatgagacgcttcggcagtccctcat |
| | cggcctgttcatgctgctctatgcccagacggtcaagaactgtgttcggcttcgtctggatgcact |
| | catccaacgggctgacgggcgttatcgcatagcatttggccgcgcggaaatccacctggatgc |
| | tcggatcagcgcgatactcgatcgctatttgaacgtgcggcaaacactggttgctctggaccat |
| | gccgacgaaaatccgtatctctttccgggacggcagttaggcagtcacctgagtgccgaatcg |
| | gtatccggttggttgaaaaaggcgggcgtcagctccgatcagttgtttgcaacatcaatctataa |
| | cgcctatcaaaatggcctgcgacttcccaaggtcttggtgcgagcgtttggcatcaccgatacg |
| | accgcgatcaagtatctcaacatgctggatccccgcctggtcatggagatcgaacgcaaacca |
| | ctgggcggcggggacgaaccctgacgcgtggggcgtctccggatcgttgaggggcaaaag |
| | catcgcggcccccgaggctgctgccaccgctgagcaataacctaggctgctgccaccgctga |
| | gcaataactagcataaccccttggggcctctaaacgggtcttgaggggttttttgctgaaacctca |
| | ggcatttgagaagcacacggtcacactgcttccggtagtcaataaaccggtaaaccagcaata |
| | gacataagcggctatttaacgaccctgccctgaaccgacgaccgggtcatcgtggccggatctt |
| | gcggcccctcggcttgaacgaattgttagacattatttgccgactaccttggtgatctcgcctttca |
| | cgtagtggacaaattcttccaactgatctgcgcgcgaggccaagcgatcttcttcttgtccaagat |
| | aagcctgtctagcttcaagtatgacgggctgatactgggccggcaggcgctccattgcccagtc |
| | ggcagcgacatccttcggcgcgattttgccggttactgcgctgtaccaaatgcgggacaacgta |
| | agcactacatttcgctcatcgccagcccagtcgggcggcgagttccatagcgttaaggtttcattt |
| | agcgcctcaaatagatcctgttcaggaaccggatcaaagagttcctccgccgctggacctacca |
| | aggcaacgctatgttctcttgcttttgtcagcaagatagccagatcaatgtcgatcgtggctggct |
| | cgaagatacctgcaagaatgtcattgcgctgccattctccaaattgcagttcgcgcttagctggat |
| | aacgccacggaatgatgtcgtcgtgcacaacaatggtgacttctacagcgcggagaatctcgct |
| | ctctccaggggaagccgaagtttccaaaaggtcgttgatcaaagctcgccgcgttgtttcatcaa |
| | gccttacggtcaccgtaaccagcaaatcaatatcactgtgtggcttcaggccgccatccactgc |
| | ggagccgtacaaatgtacggccagcaacgtcggttcgagatggcgctcgatgacgccaacta |
| | cctctgatagttgagtcgatacttcggcgatcaccgcttccctcatactcttcctttttcaatattatt |
| | gaagcatttatcagggttattgtctcatgagcggatacatatttgaatgtatttagaaaaataaacaaa |
| | tagctagctcactcggtcgctacgctccgggcgtgagactgcggcgggcgctgcggacacat |
| | acaaagttacccacagattccgtggataagcaggggactaacatgtgaggcaaaacagcagg |
| | gccgcgccggtggcgtttttccataggctccgccctcctgccagagttcacataaacagacgct |
| | tttccggtgcatctgtgggagccgtgaggctcaaccatgaatctgacagtacgggcgaaaccc |
| | gacaggacttaaagatccccaccgtttccggcgggtcgctccctcttgcgctctcctgttccgac |
| | cctgccgtttaccggatacctgttccgcctttctcccttacgggaagtgtggcgctttctcatagct |
| | cacacactggtatctcggctcggtgtaggtcgttcgctccaagctgggctgtaagcaagaactc |
| | cccgttcagcccgactgctgcgccttatccggtaactgttcacttgagtccaacccggaaaagc |
| | acggtaaaacgccactggcagcagccattggtaactgggagttcgcagaggatttgtttagcta |
| | aacacgcggttgctcttgaagtgtgcgccaaagtccggctacactggaaggacagatttggttg |
| | ctgtgctctgcgaaagccagttaccacggttaagcagttccccaactgacttaaccttcgatcaa |
| | accacctccccaggtggttttttcgtttacagggcaaaagattacgcgcagaaaaaaaggatctc |
| | aagaagatcctttgatcttttctactgaaccgctctagatttcagtgcaatttatctcttcaaatgtag |
| | cacctgaagtcagccccatacgatataagttgtaattctcatgttagtcatgccccgcgcccacc |
| | ggaaggagctgactgggttgaaggctctcaagggcatcggtcgagatcccggtgcctaatga |
| | gtgagctaacttacattaattgcgttgcgc (SEQ ID NO: 115) |
| pHelper(ZooTsy)_ | tttacactttatgcttccggctcgtatgttggtcgacgtggagatataccatgtttaactttaataagg |
| YRec-Y341F | agatatacatggcgaatccgaagatagtgctggcgacatccgatgtcgcttcgcgcgctctcgc |
| | gccagcagatgccgaactgttcgggaccatcattgatgcatggactcgtcgccgcatcggctc |
| | gctgcgccatacccacaaagccgtagccagggacgtctcggtgctgcgggagtttgctgccc |
| | actcaggtctgccgccttggctctggaatgaggatgcatttgactgctggtgcgatcatctcgga |
| | ttggagcgccggttggccaatagcacccagcggcattaccaatgctcgatccgatccttcctcg |
| | aatatctcacgacgaacgtcaagttcaggaacgagatccggcgcttgtatggcgtcgagccac |
| | gacagatttgcacggaagagaactgcatcccccataccgtcgaaaaggagtcctccaaagag |
| | cgccgagccttcacacatgaggaaatcgagttgttttttgacgggctgcggcgcgcttctgtcga |
| | ggccggccgcttcaggaccaaggattacaaccctcttcgtcgggaccgtactttctttttcaccct |
| | ctactccggagggctcaggatttccgaaggcctcaacctaaacgtaaattcgtttgcgccgaatc |
| | cagctttacctgaacttggcccctatggattcatcaaggtctggggcaagggaagccggggaa |
| | gtgggaagcgctttcggaccgtaccggtcacccatcctgacttccctgagctcatggactggta |
| | catcacaagagtccgccctgaattccttcatcgagcggaccccaatgaggaagccttgttcctct |
| | cggagcggggaactcgtctcaagtcgtccagcgccgacgcacgcttcaaaatcgcaactcgc |
| | tattgcggcctggaaggcgaaggactgacgccacactgtctgcgccactcgagcgttacccac |
| | gaatccttgcgcttcagcatcgagatggttcgtcggaagcatggccacgcttctgcggcaacga |
| | cacaaggcttcatgcacgtccccgacgttatggtcgacgacgaaatcagcagcgtcgtctccc |
| | gcgagctcgatgacgcgctcaacacacaggagaagaaagaatgagcgatacgcccaatatc |
| | agacttggttggcgcctccgcgtgcggatggcggaaaaccgaataccgaccgccacggaact |
| | caagcgacggctcgacgcgatcggctacgacatcaccagcgcccaactctcccgaattgtgg |
| | acgagcgaccggcccaggtgaaaaccgcgctactggaggctttgcttaaagtcctgggggga |
| | accctagacgatctcatgcccgcggagggccgtgttgagcagccgagttctgcgcctcaaggt |
| | ggtgaggaagttgcgccaagcccagcgccggcggccacccccacccgtaagcgcaaaccg |
| | gcgcgccccgcctcggaagcagtcgatccggacgatgccggtggccccagggtacgcccct |
| | tctcggtcccaccgaagaagtgaaatagcgttcctacatggcgacctctgctcatacctgttgcg |
| | atgaatgcagccgtccgatggccaaggcgcgtctcgtccggaacgggaaggccttgtgcgac |
| | acctgcagccagcggattccctacgccccttgtggcaagtgcggaaagcagacgcgcatgct |
| | caatgaccaaggggaaccgctctgcaaggtctgccgtgccctcaaccgcacctgcgtgcgttg |
| | cggcaagccagtgcgcaaggctgggcgcttggtcgatggcggtgctgtctgcggcccctgcg |
| | ccaagtattaccgtgaaccgcaggcctgcgatcaatgtggggcaatggagaaggttcttaccg |
| | gcgtacagacagaaactgggcggcgccgtctctgcggcaggtgtttccggcgccataaaggc |
| | tacaagagctgcagcatctgccggaaagacaggctgcctgtcggcgtgaccgctgacggcaa |
| | gcctatatgcaaaacctgtttgagtgcggccggaaaacctttcgtgtgcccaacctgtggccag |
| | gaaggaatacgccatagtagaaggcgttgccagaagtgctacgttcgcgatacggcgaaaag |
| | ccgtgtcgaggaatatgctcccctttttagtcatgcatggacccgggaagcctggctgggatttg |
| | gtgcagagaccctggaggcaatgaatccagaaaagctagtgcatcgaatcaacagctactacc |
| | tcttcttcgtccggcttgatctcgccttcagcaaacctacagaaatcacgccggtcgcgctgttga |
| | aaagcgtcggcggcttggatgggctccgtcgatacaccgtgccctatggctatctcctgaagca |
| | ccgaatcatccccgagacaacgcgcagtctcctgcaggacgaggctgaatccatcaggcagg |
| | aagccatgatcgccaagctggatggcaagtggtatgccccagtcatggcacgctaccggaatc |
| | acctgctgactgtccgcgcccgatacgacagccggggctggaaaggggaagatacccggat |
| | gaaggcgcgctcgattacgctcaacctccgggcggctcggcgtttatgcgaaacgctggatgc |
| | cataggtgtccagatgattcagcagacgacgccagagatgctggacaagttcttcatggactat |
| | cctggactggccgtctcaatccgtgccttcgtccgatacctgaaccggaaggaaaaactctttca |
| | gcgcctgcgcctgaagacagttgcccggagtgtgccggaagggattttcctgccccgcgaaa |
| | aatccactgcgctcattcaagcgtggatgacgccgactgatgagacgcttcggcagtccctcat |
| | cggcctgttcatgctgctctatgcccagacggtcaagaactgtgttcggcttcgtctggatgcact |
| | catccaacgggctgacgggcgttatcgcatagcatttggccgcgcggaaatccacctggatgc |
| | tcggatcagcgcgatactcgatcgctatttgaacgtgcggcaaacactggttgctctggaccat |
| | gccgacgaaaatccgtatctctttccgggacggcagttaggcagtcacctgagtgccgaatcg |
| | gtatccggttggttgaaaaaggcgggcgtcagctccgatcagttgtttgcaacatcaatctataa |
| | cgcctatcaaaatggcctgcgacttcccaaggtcttggtgcgagcgtttggcatcaccgatacg |
| | accgcgatcaagtatctcaacatgctggatccccgcctggtcatggagatcgaacgcaaacca |
| | ctgggcggcggggacgaaccctgatggctggcgaacttaccgttcggaaaaatccatacgcc |
| | agcggtcagccgtggtggctctatatgctcgaatgcaagggcggaggcatctacaccggcatc |
| | gcgctggatgtggatgcgcggtttgagcaacacgtcgcaggcaagggcgccaaatacacgc |
| | gcatgaaccggcccatccgggtcgtcgcgcgcatccgctacgacacgcaccgggaagccgc |
| | tcaggctgaagttgcgctgaaacgcctcaagcccctggaaaagctgcggtgggcctatgcctt |
| | ggctgggttagtcggcatggacaagtcggcgtaacgcgtggggcgtctccggatcgttgagg |
| | ggcaaaagcatcgcggcccccgaggctgctgccaccgctgagcaataacctaggctgctgcc |
| | accgctgagcaataactagcataaccccttggggcctctaaacgggtcttgaggggttttttgct |
| | gaaacctcaggcatttgagaagcacacggtcacactgcttccggtagtcaataaaccggtaaac |
| | cagcaatagacataagcggctatttaacgaccctgccctgaaccgacgaccgggtcatcgtgg |
| | ccggatcttgcggcccctcggcttgaacgaattgttagacattatttgccgactaccttggtgatct |
| | cgcctttcacgtagtggacaaattcttccaactgatctgcgcgcgaggccaagcgatcttcttctt |
| | gtccaagataagcctgtctagcttcaagtatgacgggctgatactgggccggcaggcgctccat |
| | tgcccagtcggcagcgacatccttcggcgcgattttgccggttactgcgctgtaccaaatgcgg |
| | gacaacgtaagcactacatttcgctcatcgccagcccagtcgggcggcgagttccatagcgtta |
| | aggtttcatttagcgcctcaaatagatcctgttcaggaaccggatcaaagagttcctccgccgct |
| | ggacctaccaaggcaacgctatgttctcttgcttttgtcagcaagatagccagatcaatgtcgatc |
| | gtggctggctcgaagatacctgcaagaatgtcattgcgctgccattctccaaattgcagttcgcg |
| | cttagctggataacgccacggaatgatgtcgtcgtgcacaacaatggtgacttctacagcgcgg |
| | agaatctcgctctctccaggggaagccgaagtttccaaaaggtcgttgatcaaagctcgccgcg |
| | ttgtttcatcaagccttacggtcaccgtaaccagcaaatcaatatcactgtgtggcttcaggccgc |
| | catccactgcggagccgtacaaatgtacggccagcaacgtcggttcgagatggcgctcgatga |
| | cgccaactacctctgatagttgagtcgatacttcggcgatcaccgcttccctcatactcttccttttt |
| | caatattattgaagcatttatcagggttattgtctcatgagcggatacatatttgaatgtatttagaaa |
| | aataaacaaatagctagctcactcggtcgctacgctccgggcgtgagactgcggcgggcgct |
| | gcggacacatacaaagttacccacagattccgtggataagcaggggactaacatgtgaggca |
| | aaacagcagggccgcgccggtggcgtttttccataggctccgccctcctgccagagttcacata |
| | aacagacgcttttccggtgcatctgtgggagccgtgaggctcaaccatgaatctgacagtacgg |
| | gcgaaacccgacaggacttaaagatccccaccgtttccgggggtcgctccctcttgcgctctc |
| | ctgttccgaccctgccgtttaccggatacctgttccgcctttctcccttacgggaagtgtggcgctt |
| | tctcatagctcacacactggtatctcggctcggtgtaggtcgttcgctccaagctgggctgtaag |
| | caagaactccccgttcagcccgactgctgcgccttatccggtaactgttcacttgagtccaaccc |
| | ggaaaagcacggtaaaacgccactggcagcagccattggtaactgggagttcgcagaggatt |
| | tgtttagctaaacacgcggttgctcttgaagtgtgcgccaaagtccggctacactggaaggaca |
| | gatttggttgctgtgctctgcgaaagccagttaccacggttaagcagttccccaactgacttaacc |
| | ttcgatcaaaccacctccccaggtggttttttcgtttacagggcaaaagattacgcgcagaaaaa |
| | aaggatctcaagaagatcctttgatcttttctactgaaccgctctagatttcagtgcaatttatctctt |
| | caaatgtagcacctgaagtcagccccatacgatataagttgtaattctcatgttagtcatgccccg |
| | cgcccaccggaaggagctgactgggttgaaggctctcaagggcatcggtcgagatcccggtg |
| | cctaatgagtgagctaacttacattaattgcgttgcgc (SEQ ID NO: 116) |
| pHelper(ZooTsy)_ | tttacactttatgcttccggctcgtatgttggtcgacgtggagatataccatgtttaactttaataagg |
| TnsF-Y584F | agatatacatggcgaatccgaagatagtgctggcgacatccgatgtcgcttcgcgcgctctcgc |
| | gccagcagatgccgaactgttcgggaccatcattgatgcatggactcgtcgccgcatcggctc |
| | gctgcgccatacccacaaagccgtagccagggacgtctcggtgctgcgggagtttgctgccc |
| | actcaggtctgccgccttggctctggaatgaggatgcatttgactgctggtgcgatcatctcgga |
| | ttggagcgccggttggccaatagcacccagcggcattaccaatgctcgatccgatccttcctcg |
| | aatatctcacgacgaacgtcaagttcaggaacgagatccggcgcttgtatggcgtcgagccac |
| | gacagatttgcacggaagagaactgcatcccccataccgtcgaaaaggagtcctccaaagag |
| | cgccgagccttcacacatgaggaaatcgagttgttttttgacgggctgcggcgcgcttctgtcga |
| | ggccggccgcttcaggaccaaggattacaaccctcttcgtcgggaccgtactttctttttcaccct |
| | ctactccggagggctcaggatttccgaaggcctcaacctaaacgtaaattcgtttgcgccgaatc |
| | cagctttacctgaacttggcccctatggattcatcaaggtctggggcaagggaagccggggaa |
| | gtgggaagcgctttcggaccgtaccggtcacccatcctgacttccctgagctcatggactggta |
| | catcacaagagtccgccctgaattccttcatcgagcggaccccaatgaggaagccttgttcctct |
| | cggagcggggaactcgtctcaagtcgtccagcgccgacgcacgcttcaaaatcgcaactcgc |
| | tattgcggcctggaaggcgaaggactgacgccacactgtctgcgccactcgagcgttacccac |
| | gaatccttgcgcttcagcatcgagatggttcgtcggaagcatggccacgcttctgcggcaacga |
| | cacaaggctatatgcacgtccccgacgttatggtcgacgacgaaatcagcagcgtcgtctccc |
| | gcgagctcgatgacgcgctcaacacacaggagaagaaagaatgagcgatacgcccaatatc |
| | agacttggttggcgcctccgcgtgcggatggcggaaaaccgaataccgaccgccacggaact |
| | caagcgacggctcgacgcgatcggctacgacatcaccagcgcccaactctcccgaattgtgg |
| | acgagcgaccggcccaggtgaaaaccgcgctactggaggctttgcttaaagtcctgggggga |
| | accctagacgatctcatgcccgcggagggccgtgttgagcagccgagttctgcgcctcaaggt |
| | ggtgaggaagttgcgccaagcccagcgccggcggccacccccacccgtaagcgcaaaccg |
| | gcgcgccccgcctcggaagcagtcgatccggacgatgccggtggccccagggtacgcccct |
| | tctcggtcccaccgaagaagtgaaatagcgttcctacatggcgacctctgctcatacctgttgcg |
| | atgaatgcagccgtccgatggccaaggcgcgtctcgtccggaacgggaaggccttgtgcgac |
| | acctgcagccagcggattccctacgccccttgtggcaagtgcggaaagcagacgcgcatgct |
| | caatgaccaaggggaaccgctctgcaaggtctgccgtgccctcaaccgcacctgcgtgcgttg |
| | cggcaagccagtgcgcaaggctgggcgcttggtcgatggcggtgctgtctgcggcccctgcg |
| | ccaagtattaccgtgaaccgcaggcctgcgatcaatgtggggcaatggagaaggttcttaccg |
| | gcgtacagacagaaactgggcggcgccgtctctgcggcaggtgtttccggcgccataaaggc |
| | tacaagagctgcagcatctgccggaaagacaggctgcctgtcggcgtgaccgctgacggcaa |
| | gcctatatgcaaaacctgtttgagtgcggccggaaaacctttcgtgtgcccaacctgtggccag |
| | gaaggaatacgccatagtagaaggcgttgccagaagtgctacgttcgcgatacggcgaaaag |
| | ccgtgtcgaggaatatgctcccctttttagtcatgcatggacccgggaagcctggctgggatttg |
| | gtgcagagaccctggaggcaatgaatccagaaaagctagtgcatcgaatcaacagctactacc |
| | tcttcttcgtccggcttgatctcgccttcagcaaacctacagaaatcacgccggtcgcgctgttga |
| | aaagcgtcggcggcttggatgggctccgtcgatacaccgtgccctatggctatctcctgaagca |
| | ccgaatcatccccgagacaacgcgcagtctcctgcaggacgaggctgaatccatcaggcagg |
| | aagccatgatcgccaagctggatggcaagtggtatgccccagtcatggcacgctaccggaatc |
| | acctgctgactgtccgcgcccgatacgacagccggggctggaaaggggaagatacccggat |
| | gaaggcgcgctcgattacgctcaacctccgggcggctcggcgtttatgcgaaacgctggatgc |
| | cataggtgtccagatgattcagcagacgacgccagagatgctggacaagttcttcatggactat |
| | cctggactggccgtctcaatccgtgccttcgtccgatacctgaaccggaaggaaaaactctttca |
| | gcgcctgcgcctgaagacagttgcccggagtgtgccggaagggattttcctgccccgcgaaa |
| | aatccactgcgctcattcaagcgtggatgacgccgactgatgagacgcttcggcagtccctcat |
| | cggcctgttcatgctgctctatgcccagacggtcaagaactgtgttcggcttcgtctggatgcact |
| | catccaacgggctgacgggcgttatcgcatagcatttggccgcgcggaaatccacctggatgc |
| | tcggatcagcgcgatactcgatcgctatttgaacgtgcggcaaacactggttgctctggaccat |
| | gccgacgaaaatccgtatctctttccgggacggcagttaggcagtcacctgagtgccgaatcg |
| | gtatccggttggttgaaaaaggcgggcgtcagctccgatcagttgtttgcaacatcaatctataa |
| | cgcctatcaaaatggcctgcgacttcccaaggtcttggtgcgagcgtttggcatcaccgatacg |
| | accgcgatcaagttcctcaacatgctggatccccgcctggtcatggagatcgaacgcaaacca |
| | ctgggcggcggggacgaaccctgatggctggcgaacttaccgttcggaaaaatccatacgcc |
| | agcggtcagccgtggtggctctatatgctcgaatgcaagggcggaggcatctacaccggcatc |
| | gcgctggatgtggatgcgcggtttgagcaacacgtcgcaggcaagggcgccaaatacacgc |
| | gcatgaaccggcccatccgggtcgtcgcgcgcatccgctacgacacgcaccgggaagccgc |
| | tcaggctgaagttgcgctgaaacgcctcaagcccctggaaaagctgcggtgggcctatgcctt |
| | ggctgggttagtcggcatggacaagtcggcgtaacgcgtggggcgtctccggatcgttgagg |
| | ggcaaaagcatcgcggcccccgaggctgctgccaccgctgagcaataacctaggctgctgcc |
| | accgctgagcaataactagcataaccccttggggcctctaaacgggtcttgaggggttttttgct |
| | gaaacctcaggcatttgagaagcacacggtcacactgcttccggtagtcaataaaccggtaaac |
| | cagcaatagacataagcggctatttaacgaccctgccctgaaccgacgaccgggtcatcgtgg |
| | ccggatcttgcggcccctcggcttgaacgaattgttagacattatttgccgactaccttggtgatct |
| | cgcctttcacgtagtggacaaattcttccaactgatctgcgcgcgaggccaagcgatcttcttctt |
| | gtccaagataagcctgtctagcttcaagtatgacgggctgatactgggccggcaggcgctccat |
| | tgcccagtcggcagcgacatccttcggcgcgattttgccggttactgcgctgtaccaaatgcgg |
| | gacaacgtaagcactacatttcgctcatcgccagcccagtcgggcggcgagttccatagcgtta |
| | aggtttcatttagcgcctcaaatagatcctgttcaggaaccggatcaaagagttcctccgccgct |
| | ggacctaccaaggcaacgctatgttctcttgcttttgtcagcaagatagccagatcaatgtcgatc |
| | gtggctggctcgaagatacctgcaagaatgtcattgcgctgccattctccaaattgcagttcgcg |
| | cttagctggataacgccacggaatgatgtcgtcgtgcacaacaatggtgacttctacagcgcgg |
| | agaatctcgctctctccaggggaagccgaagtttccaaaaggtcgttgatcaaagctcgccgcg |
| | ttgtttcatcaagccttacggtcaccgtaaccagcaaatcaatatcactgtgtggcttcaggccgc |
| | catccactgcggagccgtacaaatgtacggccagcaacgtcggttcgagatggcgctcgatga |
| | cgccaactacctctgatagttgagtcgatacttcggcgatcaccgcttccctcatactcttccttttt |
| | caatattattgaagcatttatcagggttattgtctcatgagcggatacatatttgaatgtatttagaaa |
| | aataaacaaatagctagctcactcggtcgctacgctccgggcgtgagactgcggcgggcgct |
| | gcggacacatacaaagttacccacagattccgtggataagcaggggactaacatgtgaggca |
| | aaacagcagggccgcgccggtggcgtttttccataggctccgccctcctgccagagttcacata |
| | aacagacgcttttccggtgcatctgtgggagccgtgaggctcaaccatgaatctgacagtacgg |
| | gcgaaacccgacaggacttaaagatccccaccgtttccgggggtcgctccctcttgcgctctc |
| | ctgttccgaccctgccgtttaccggatacctgttccgcctttctcccttacgggaagtgtggcgctt |
| | tctcatagctcacacactggtatctcggctcggtgtaggtcgttcgctccaagctgggctgtaag |
| | caagaactccccgttcagcccgactgctgcgccttatccggtaactgttcacttgagtccaaccc |
| | ggaaaagcacggtaaaacgccactggcagcagccattggtaactgggagttcgcagaggatt |
| | tgtttagctaaacacgcggttgctcttgaagtgtgcgccaaagtccggctacactggaaggaca |
| | gatttggttgctgtgctctgcgaaagccagttaccacggttaagcagttccccaactgacttaacc |
| | ttcgatcaaaccacctccccaggtggttttttcgtttacagggcaaaagattacgcgcagaaaaa |
| | aaggatctcaagaagatcctttgatcttttctactgaaccgctctagatttcagtgcaatttatctctt |
| | caaatgtagcacctgaagtcagccccatacgatataagttgtaattctcatgttagtcatgccccg |
| | cgcccaccggaaggagctgactgggttgaaggctctcaagggcatcggtcgagatcccggtg |
| | cctaatgagtgagctaacttacattaattgcgttgcgc (SEQ ID NO: 117) |
| pR6K- | gagagtgcaccatacaggaaacagctatgaccatgattacgccaagctcggcgcgccattgg |
| Donor(ZooTsy)_ | gattcggtcgaattcggactgggattctccaatattccttagcgcttccaataaagagcgcttttga |
| H1:12_E1:135_E2:39_ | agctgatgtgcttaaaaacttactcaatggaataattctagataattcactagtgattggaaatgttg |
| H2:12 | gatcctcatactcttcctttttcaattcagaagaactcgtcaagaaggcgatagaaggcgatgcg |
| | ctgcgaatcgggagcggcgataccgtaaagcacgaggaagcggtcagcccattcgccgcca |
| | agctcttcagcaatatcacgggtagccaacgctatgtcctgatagcggtccgccacacccagcc |
| | ggccacagtcgatgaatccagaaaagcggccattttccaccatgatattcggcaagcaggcat |
| | cgccatgggtcacgacgagatcctcgccgtcgggcatgcgcgccttgagcctggcgaacagt |
| | tcggctggcgcgagcccctgatgctcttcgtccagatcatcctgatcgacaagaccggcttcca |
| | tccgagtacgtgctcgctcgatgcgatgtttcgcttggtggtcgaatgggcaggtagccggatc |
| | aagcgtatgcagccgccgcattgcatcagccatgatggatactttctcggcaggagcaaggtg |
| | ggatgacaggagatcctgccccggcacttcgcccaatagcagccagtcccttcccgcttcagt |
| | gacaacgtcgagcacagctgcgcaaggaacgcccgtcgtggccagccacgatagccgcgct |
| | gcctcgtcctgcagttcattcagggcaccggacaggtcggtcttgacaaaaagaaccgggcgc |
| | ccctgcgctgacagccggaacacggcggcatcagagcagccgattgtctgttgtgcccagtca |
| | tagccgaatagcctctccacccaagcggccggagaacctgcgtgcaatccatcttgttcaatca |
| | tgcgaaacgatcctcatcctgtctcttgatcagatcttgatcccctgcgccatcagatccttggcg |
| | gcaagaaagccatccagtttactttgcagggcttcccaaccttaccagagggcgccccagctg |
| | gcaattccggttcgcttgctgtccataaaaccgcccagtctagctatcgccatgtaagcccactg |
| | caagctacctgctttctctttgcgcttgcgttttcccttgtccagatagcccagtagctgacattcat |
| | ccggggtcagcaccgtttctgcggactggctttctacgtgttccgcttcctttagcagcccttgcg |
| | ccatgcatttgttaaatcgaattatccattgctgttgacaaagggaatcaggggatcttgaagttcc |
| | tattccgaagttcctattctctagaaagtataggaacttcagagcgcttttgaagctaattcgagat |
| | catgcatgagctcactagtggatccccccgactacttctaatgctgtgacatcactcacaggattc |
| | ctgcaagtaatgcgacattggatactttgggattcctttatagtcagtgggttatccgaatttttgcc |
| | ttgttcgttgctacattggctaaatcagaacttcggcagctcgtcatcccaatacgcgtcaattcac |
| | tggccgtcgttttactactttccgctgcataaccctgcttcggggtcattatagcgattttttcggtat |
| | atccatcctttttcgcacgatatacaggattttgccaaagggttcgtgtagactttccttggtgtatc |
| | caacggcgtcagccgggcaggataggtgaagtaggcccacccgcgagcgggtgttccttctt |
| | cactgtcccttattcgcacctggcggtgctcaacgggaatcctgctctgcgaggctggccgata |
| | agctctgataacgcaggaaagaacatgggcagttcaacctgttgatagtacgtactaagctctca |
| | tgtttcacgtactaagctctcaagtttaacgtactaagctctcatgtttaacgaactaaaccctcatg |
| | gctaacgtactaagctctcatggctaacgtactaagctctcatgtttcacgtactaagctctcatgtt |
| | tgaacaataaaattaatataaatcagcaacttaaatagcctctaaggttttaagttttataagaaaaa |
| | aaagaatatataaggcttttaaagcttttaaggtttaacggttgtggacaacaagccagggatgta |
| | acgcactgagaagcccttagagcctctcaaagcaattttgagtgacacaggaacacttaacggc |
| | tgacaaccggctggtaacaggattagcagagcgaggtatgtaggcggtgctacaagattgtac |
| | tgagagtgcaccatacaggaaacagc (SEQ ID NO: 118) |
| pR6K- | gagagtgcaccatacaggaaacagctatgaccatgattacgccaagctcggcgcgccattgg |
| Donor(ZooTsy)_H1:0_ | gatggactgggattctccaatattccttagcgcttccaataaagagcgcttttgaagctgatgtgct |
| E1:135_E2:39_H2:0 | taaaaacttactcaatggaataattctagataattcactagtgattggaaatgttggatcctcatact |
| | cttcctttttcaattcagaagaactcgtcaagaaggcgatagaaggcgatgcgctgcgaatcgg |
| | gagcggcgataccgtaaagcacgaggaagcggtcagcccattcgccgccaagctcttcagca |
| | atatcacgggtagccaacgctatgtcctgatagcggtccgccacacccagccggccacagtcg |
| | atgaatccagaaaagcggccattttccaccatgatattcggcaagcaggcatcgccatgggtca |
| | cgacgagatcctcgccgtcgggcatgcgcgccttgagcctggcgaacagttcggctggcgcg |
| | agcccctgatgctcttcgtccagatcatcctgatcgacaagaccggcttccatccgagtacgtgc |
| | tcgctcgatgcgatgtttcgcttggtggtcgaatgggcaggtagccggatcaagcgtatgcagc |
| | cgccgcattgcatcagccatgatggatactttctcggcaggagcaaggtgggatgacaggaga |
| | tcctgccccggcacttcgcccaatagcagccagtcccttcccgcttcagtgacaacgtcgagca |
| | cagctgcgcaaggaacgcccgtcgtggccagccacgatagccgcgctgcctcgtcctgcagt |
| | tcattcagggcaccggacaggtcggtcttgacaaaaagaaccgggcgcccctgcgctgacag |
| | ccggaacacggggcatcagagcagccgattgtctgttgtgcccagtcatagccgaatagcct |
| | ctccacccaagcggccggagaacctgcgtgcaatccatcttgttcaatcatgcgaaacgatcct |
| | catcctgtctcttgatcagatcttgatcccctgcgccatcagatccttggcggcaagaaagccatc |
| | cagtttactttgcagggcttcccaaccttaccagagggcgccccagctggcaattccggttcgct |
| | tgctgtccataaaaccgcccagtctagctatcgccatgtaagcccactgcaagctacctgctttct |
| | ctttgcgcttgcgttttcccttgtccagatagcccagtagctgacattcatccggggtcagcaccg |
| | tttctgcggactggctttctacgtgttccgcttcctttagcagcccttgcgccatgcatttgttaaatc |
| | gaattatccattgctgttgacaaagggaatcaggggatcttgaagttcctattccgaagttcctatt |
| | ctctagaaagtataggaacttcagagcgcttttgaagctaattcgagatcatgcatgagctcacta |
| | gtggatccccccgactacttctaatgctgtgacatcactcacaggattcctgcaagtaatgcgac |
| | attggatactttgggattcctttatagtcagtgggttatccgaatttttgccttgttcgttgctacatt |
| | ggctaaatcagaacttatcccaatacgcgtcaattcactggccgtcgttttactactttccgctgcat |
| | aaccctgcttcggggtcattatagcgattttttcggtatatccatcctttttcgcacgatatacagga |
| | ttttgccaaagggttcgtgtagactttccttggtgtatccaacggcgtcagccgggcaggatagg |
| | tgaagtaggcccacccgcgagcgggtgttccttcttcactgtcccttattcgcacctggcggtgc |
| | tcaacgggaatcctgctctgcgaggctggccgataagctctgataacgcaggaaagaacatgg |
| | gcagttcaacctgttgatagtacgtactaagctctcatgtttcacgtactaagctctcaagtttaacg |
| | tactaagctctcatgtttaacgaactaaaccctcatggctaacgtactaagctctcatggctaacgt |
| | actaagctctcatgtttcacgtactaagctctcatgtttgaacaataaaattaatataaatcagcaac |
| | ttaaatagcctctaaggttttaagttttataagaaaaaaaagaatatataaggcttttaaagcttttaa |
| | ggtttaacggttgtggacaacaagccagggatgtaacgcactgagaagcccttagagcctctc |
| | aaagcaattttgagtgacacaggaacacttaacggctgacaaccggctggtaacaggattagc |
| | agagcgaggtatgtaggcggtgctacaagattgtactgagagtgcaccatacaggaaacagc |
| | (SEQ ID NO: 119) |
| pR6K- | gagagtgcaccatacaggaaacagctatgaccatgattacgccaagctcggcgcgccattgg |
| Donor(ZooTsy)_H1:0_ | gatagagcgcttttgaagctgatgtgcttaaaaacttactcaatggaataattctagataattcacta |
| E1:0_E2:0_H2:0 | gtgattggaaatgttggatcctcatactcttcctttttcaattcagaagaactcgtcaagaaggcga |
| | tagaaggcgatgcgctgcgaatcgggagcggcgataccgtaaagcacgaggaagcggtca |
| | gcccattcgccgccaagctcttcagcaatatcacgggtagccaacgctatgtcctgatagcggt |
| | ccgccacacccagccggccacagtcgatgaatccagaaaagcggccattttccaccatgatat |
| | tcggcaagcaggcatcgccatgggtcacgacgagatcctcgccgtcgggcatgcgcgccttg |
| | agcctggcgaacagttcggctggcgcgagcccctgatgctcttcgtccagatcatcctgatcga |
| | caagaccggcttccatccgagtacgtgctcgctcgatgcgatgtttcgcttggtggtcgaatgg |
| | gcaggtagccggatcaagcgtatgcagccgccgcattgcatcagccatgatggatactttctcg |
| | gcaggagcaaggtgggatgacaggagatcctgccccggcacttcgcccaatagcagccagt |
| | cccttcccgcttcagtgacaacgtcgagcacagctgcgcaaggaacgcccgtcgtggccagc |
| | cacgatagccgcgctgcctcgtcctgcagttcattcagggcaccggacaggtcggtcttgaca |
| | aaaagaaccgggcgcccctgcgctgacagccggaacacggcggcatcagagcagccgatt |
| | gtctgttgtgcccagtcatagccgaatagcctctccacccaagcggccggagaacctgcgtgc |
| | aatccatcttgttcaatcatgcgaaacgatcctcatcctgtctcttgatcagatcttgatcccctgcg |
| | ccatcagatccttggcggcaagaaagccatccagtttactttgcagggcttcccaaccttaccag |
| | agggcgccccagctggcaattccggttcgcttgctgtccataaaaccgcccagtctagctatcg |
| | ccatgtaagcccactgcaagctacctgctttctctttgcgcttgcgttttcccttgtccagatagccc |
| | agtagctgacattcatccggggtcagcaccgtttctgcggactggctttctacgtgttccgcttcc |
| | tttagcagcccttgcgccatgcatttgttaaatcgaattatccattgctgttgacaaagggaatcag |
| | gggatcttgaagttcctattccgaagttcctattctctagaaagtataggaacttcagagcgctttt |
| | gaagctaattcgagatcatgcatgagctcactagtggatccccccgacatcccaatacgcgtca |
| | attcactggccgtcgttttactactttccgctgcataaccctgcttcggggtcattatagcgatttttt |
| | cggtatatccatcctttttcgcacgatatacaggattttgccaaagggttcgtgtagactttccttgg |
| | tgtatccaacggcgtcagccgggcaggataggtgaagtaggcccacccgcgagcgggtgttc |
| | cttcttcactgtcccttattcgcacctggcggtgctcaacgggaatcctgctctgcgaggctggc |
| | cgataagctctgataacgcaggaaagaacatgggcagttcaacctgttgatagtacgtactaag |
| | ctctcatgtttcacgtactaagctctcaagtttaacgtactaagctctcatgtttaacgaactaaacc |
| | ctcatggctaacgtactaagctctcatggctaacgtactaagctctcatgtttcacgtactaagctc |
| | tcatgtttgaacaataaaattaatataaatcagcaacttaaatagcctctaaggttttaagttttataa |
| | gaaaaaaaagaatatataaggcttttaaagcttttaaggtttaacggttgtggacaacaagccag |
| | ggatgtaacgcactgagaagcccttagagcctctcaaagcaattttgagtgacacaggaacact |
| | taacggctgacaaccggctggtaacaggattagcagagcgaggtatgtaggcggtgctacaa |
| | gattgtactgagagtgcaccatacaggaaacagc (SEQ ID NO: 120) |
| pR6K- | gagagtgcaccatacaggaaacagctatgaccatgattacgccaagctcggcgcgccattgg |
| Donor(ZooTsy)_ | gattcggtcgaattcggactgggattctccaatattccttagcgcttccaataaagagcgcttttga |
| H1:12_ | agctgatgtgcttaaaaacttactcaatggaataattctagataattcactagtgattggaaatgttg |
| E1:85_E2:39_H2:12 | gatcctcatactcttcctttttcaattcagaagaactcgtcaagaaggcgatagaaggcgatgcg |
| | ctgcgaatcgggagcggcgataccgtaaagcacgaggaagcggtcagcccattcgccgcca |
| | agctcttcagcaatatcacgggtagccaacgctatgtcctgatagcggtccgccacacccagcc |
| | ggccacagtcgatgaatccagaaaagcggccattttccaccatgatattcggcaagcaggcat |
| | cgccatgggtcacgacgagatcctcgccgtcgggcatgcgcgccttgagcctggcgaacagt |
| | tcggctggcgcgagcccctgatgctcttcgtccagatcatcctgatcgacaagaccggcttcca |
| | tccgagtacgtgctcgctcgatgcgatgtttcgcttggtggtcgaatgggcaggtagccggatc |
| | aagcgtatgcagccgccgcattgcatcagccatgatggatactttctcggcaggagcaaggtg |
| | ggatgacaggagatcctgccccggcacttcgcccaatagcagccagtcccttcccgcttcagt |
| | gacaacgtcgagcacagctgcgcaaggaacgcccgtcgtggccagccacgatagccgcgct |
| | gcctcgtcctgcagttcattcagggcaccggacaggtcggtcttgacaaaaagaaccgggcgc |
| | ccctgcgctgacagccggaacacggcggcatcagagcagccgattgtctgttgtgcccagtca |
| | tagccgaatagcctctccacccaagcggccggagaacctgcgtgcaatccatcttgttcaatca |
| | tgcgaaacgatcctcatcctgtctcttgatcagatcttgatcccctgcgccatcagatccttggcg |
| | gcaagaaagccatccagtttactttgcagggcttcccaaccttaccagagggcgccccagctg |
| | gcaattccggttcgcttgctgtccataaaaccgcccagtctagctatcgccatgtaagcccactg |
| | caagctacctgctttctctttgcgcttgcgttttcccttgtccagatagcccagtagctgacattcat |
| | ccggggtcagcaccgtttctgcggactggctttctacgtgttccgcttcctttagcagcccttgcg |
| | ccatgcatttgttaaatcgaattatccattgctgttgacaaagggaatcaggggatcttgaagttcc |
| | tattccgaagttcctattctctagaaagtataggaacttcagagcgcttttgaagctaattcgagat |
| | catgcatgagctcactagtggatccccccgacattggatactttgggattcctttatagtcagtgg |
| | gttatccgaatttttgccttgttcgttgctacattggctaaatcagaacttcggcagctcgtcatccc |
| | aatacgcgtcaattcactggccgtcgttttactactttccgctgcataaccctgcttcggggtcatt |
| | atagcgattttttcggtatatccatcctttttcgcacgatatacaggattttgccaaagggttcgtgta |
| | gactttccttggtgtatccaacggcgtcagccgggcaggataggtgaagtaggcccacccgcg |
| | agcgggtgttccttcttcactgtcccttattcgcacctggcggtgctcaacgggaatcctgctctg |
| | cgaggctggccgataagctctgataacgcaggaaagaacatgggcagttcaacctgttgatag |
| | tacgtactaagctctcatgtttcacgtactaagctctcaagtttaacgtactaagctctcatgtttaac |
| | gaactaaaccctcatggctaacgtactaagctctcatggctaacgtactaagctctcatgtttcac |
| | gtactaagctctcatgtttgaacaataaaattaatataaatcagcaacttaaatagcctctaaggttt |
| | taagttttataagaaaaaaaagaatatataaggcttttaaagcttttaaggtttaacggttgtggaca |
| | acaagccagggatgtaacgcactgagaagcccttagagcctctcaaagcaattttgagtgaca |
| | caggaacacttaacggctgacaaccggctggtaacaggattagcagagcgaggtatgtaggc |
| | ggtgctacaagattgtactgagagtgcaccatacaggaaacagc (SEQ ID NO: 121) |
| pR6K- | gagagtgcaccatacaggaaacagctatgaccatgattacgccaagctcggcgcgccattgg |
| Donor(ZooTsy)_ | gattcggtcgaattcggactgggattctccaatattccttagcgcttccaataaagagcgcttttga |
| H1:12_ | agctgatgtgcttaaaaacttactcaatggaataattctagataattcactagtgattggaaatgttg |
| E1:35_E2:39_H2:12 | gatcctcatactcttcctttttcaattcagaagaactcgtcaagaaggcgatagaaggcgatgcg |
| | ctgcgaatcgggagcggcgataccgtaaagcacgaggaagcggtcagcccattcgccgcca |
| | agctcttcagcaatatcacgggtagccaacgctatgtcctgatagcggtccgccacacccagcc |
| | ggccacagtcgatgaatccagaaaagcggccattttccaccatgatattcggcaagcaggcat |
| | cgccatgggtcacgacgagatcctcgccgtcgggcatgcgcgccttgagcctggcgaacagt |
| | tcggctggcgcgagcccctgatgctcttcgtccagatcatcctgatcgacaagaccggcttcca |
| | tccgagtacgtgctcgctcgatgcgatgtttcgcttggtggtcgaatgggcaggtagccggatc |
| | aagcgtatgcagccgccgcattgcatcagccatgatggatactttctcggcaggagcaaggtg |
| | ggatgacaggagatcctgccccggcacttcgcccaatagcagccagtcccttcccgcttcagt |
| | gacaacgtcgagcacagctgcgcaaggaacgcccgtcgtggccagccacgatagccgcgct |
| | gcctcgtcctgcagttcattcagggcaccggacaggtcggtcttgacaaaaagaaccgggcgc |
| | ccctgcgctgacagccggaacacggcggcatcagagcagccgattgtctgttgtgcccagtca |
| | tagccgaatagcctctccacccaagcggccggagaacctgcgtgcaatccatcttgttcaatca |
| | tgcgaaacgatcctcatcctgtctcttgatcagatcttgatcccctgcgccatcagatccttggcg |
| | gcaagaaagccatccagtttactttgcagggcttcccaaccttaccagagggcgccccagctg |
| | gcaattccggttcgcttgctgtccataaaaccgcccagtctagctatcgccatgtaagcccactg |
| | caagctacctgctttctctttgcgcttgcgttttcccttgtccagatagcccagtagctgacattcat |
| | ccggggtcagcaccgtttctgcggactggctttctacgtgttccgcttcctttagcagcccttgcg |
| | ccatgcatttgttaaatcgaattatccattgctgttgacaaagggaatcaggggatcttgaagttcc |
| | tattccgaagttcctattctctagaaagtataggaacttcagagcgcttttgaagctaattcgagat |
| | catgcatgagctcactagtggatccccccgacccttgttcgttgctacattggctaaatcagaact |
| | tcggcagctcgtcatcccaatacgcgtcaattcactggccgtcgttttactactttccgctgcataa |
| | ccctgcttcggggtcattatagcgattttttcggtatatccatcctttttcgcacgatatacaggattt |
| | tgccaaagggttcgtgtagactttccttggtgtatccaacggcgtcagccgggcaggataggtga |
| | agtaggcccacccgcgagcgggtgttccttcttcactgtcccttattcgcacctggcggtgctca |
| | acgggaatcctgctctgcgaggctggccgataagctctgataacgcaggaaagaacatgggc |
| | agttcaacctgttgatagtacgtactaagctctcatgtttcacgtactaagctctcaagtttaacgta |
| | ctaagctctcatgtttaacgaactaaaccctcatggctaacgtactaagctctcatggctaacgta |
| | ctaagctctcatgtttcacgtactaagctctcatgtttgaacaataaaattaatataaatcagcaactt |
| | aaatagcctctaaggttttaagttttataagaaaaaaaagaatatataaggcttttaaagcttttaag |
| | gtttaacggttgtggacaacaagccagggatgtaacgcactgagaagcccttagagcctctcaa |
| | agcaattttgagtgacacaggaacacttaacggctgacaaccggctggtaacaggattagcag |
| | agcgaggtatgtaggcggtgctacaagattgtactgagagtgcaccatacaggaaacagc |
| | (SEQ ID NO: 122) |
| pR6K- | gagagtgcaccatacaggaaacagctatgaccatgattacgccaagctcggcgcgccattgg |
| Donor(ZooTsy)_ | gattcggtcgaattcggactgggattctccaatattccttagcgcttccaataaagagcgcttttga |
| H1:12_ | agctgatgtgcttaaaaacttactcaatggaataattctagataattcactagtgattggaaatgttg |
| E1:0_E2:39_H2:12 | gatcctcatactcttcctttttcaattcagaagaactcgtcaagaaggcgatagaaggcgatgcg |
| | ctgcgaatcgggagcggcgataccgtaaagcacgaggaagcggtcagcccattcgccgcca |
| | agctcttcagcaatatcacgggtagccaacgctatgtcctgatagcggtccgccacacccagcc |
| | ggccacagtcgatgaatccagaaaagcggccattttccaccatgatattcggcaagcaggcat |
| | cgccatgggtcacgacgagatcctcgccgtcgggcatgcgcgccttgagcctggcgaacagt |
| | tcggctggcgcgagcccctgatgctcttcgtccagatcatcctgatcgacaagaccggcttcca |
| | tccgagtacgtgctcgctcgatgcgatgtttcgcttggtggtcgaatgggcaggtagccggatc |
| | aagcgtatgcagccgccgcattgcatcagccatgatggatactttctcggcaggagcaaggtg |
| | ggatgacaggagatcctgccccggcacttcgcccaatagcagccagtcccttcccgcttcagt |
| | gacaacgtcgagcacagctgcgcaaggaacgcccgtcgtggccagccacgatagccgcgct |
| | gcctcgtcctgcagttcattcagggcaccggacaggtcggtcttgacaaaaagaaccgggcgc |
| | ccctgcgctgacagccggaacacggcggcatcagagcagccgattgtctgttgtgcccagtca |
| | tagccgaatagcctctccacccaagcggccggagaacctgcgtgcaatccatcttgttcaatca |
| | tgcgaaacgatcctcatcctgtctcttgatcagatcttgatcccctgcgccatcagatccttggcg |
| | gcaagaaagccatccagtttactttgcagggcttcccaaccttaccagagggcgccccagctg |
| | gcaattccggttcgcttgctgtccataaaaccgcccagtctagctatcgccatgtaagcccactg |
| | caagctacctgctttctctttgcgcttgcgttttcccttgtccagatagcccagtagctgacattcat |
| | ccggggtcagcaccgtttctgcggactggctttctacgtgttccgcttcctttagcagcccttgcg |
| | ccatgcatttgttaaatcgaattatccattgctgttgacaaagggaatcaggggatcttgaagttcc |
| | tattccgaagttcctattctctagaaagtataggaacttcagagcgcttttgaagctaattcgagat |
| | catgcatgagctcactagtggatccccccgaccggcagctcgtcatcccaatacgcgtcaattc |
| | actggccgtcgttttactactttccgctgcataaccctgcttcggggtcattatagcgattttttcggt |
| | atatccatcctttttcgcacgatatacaggattttgccaaagggttcgtgtagactttccttggtgtat |
| | ccaacggcgtcagccgggcaggataggtgaagtaggcccacccgcgagcgggtgttccttct |
| | tcactgtcccttattcgcacctggcggtgctcaacgggaatcctgctctgcgaggctggccgat |
| | aagctctgataacgcaggaaagaacatgggcagttcaacctgttgatagtacgtactaagctctc |
| | atgtttcacgtactaagctctcaagtttaacgtactaagctctcatgtttaacgaactaaaccctcat |
| | ggctaacgtactaagctctcatggctaacgtactaagctctcatgtttcacgtactaagctctcatg |
| | tttgaacaataaaattaatataaatcagcaacttaaatagcctctaaggttttaagttttataagaaaa |
| | aaaagaatatataaggcttttaaagcttttaaggtttaacggttgtggacaacaagccagggatgt |
| | aacgcactgagaagcccttagagcctctcaaagcaattttgagtgacacaggaacacttaacgg |
| | ctgacaaccggctggtaacaggattagcagagcgaggtatgtaggcggtgctacaagattgta |
| | ctgagagtgcaccatacaggaaacagc (SEQ ID NO: 123) |
| pR6K- | gagagtgcaccatacaggaaacagctatgaccatgattacgccaagctcggcgcgccattgg |
| Donor(ZooTsy)_ | gattcggtcgaattcggactgggattctccaatattccttagcgcttccaataaagagcgcttttga |
| H1:12_ | agctgatgtgcttaaaaacttactcaatggaataattctagataattcactagtgattggaaatgttg |
| E1:115_E2:39_H2:12 | gatcctcatactcttcctttttcaattcagaagaactcgtcaagaaggcgatagaaggcgatgcg |
| | ctgcgaatcgggagcggcgataccgtaaagcacgaggaagcggtcagcccattcgccgcca |
| | agctcttcagcaatatcacgggtagccaacgctatgtcctgatagcggtccgccacacccagcc |
| | ggccacagtcgatgaatccagaaaagcggccattttccaccatgatattcggcaagcaggcat |
| | cgccatgggtcacgacgagatcctcgccgtcgggcatgcgcgccttgagcctggcgaacagt |
| | tcggctggcgcgagcccctgatgctcttcgtccagatcatcctgatcgacaagaccggcttcca |
| | tccgagtacgtgctcgctcgatgcgatgtttcgcttggtggtcgaatgggcaggtagccggatc |
| | aagcgtatgcagccgccgcattgcatcagccatgatggatactttctcggcaggagcaaggtg |
| | ggatgacaggagatcctgccccggcacttcgcccaatagcagccagtcccttcccgcttcagt |
| | gacaacgtcgagcacagctgcgcaaggaacgcccgtcgtggccagccacgatagccgcgct |
| | gcctcgtcctgcagttcattcagggcaccggacaggtcggtcttgacaaaaagaaccgggcgc |
| | ccctgcgctgacagccggaacacggcggcatcagagcagccgattgtctgttgtgcccagtca |
| | tagccgaatagcctctccacccaagcggccggagaacctgcgtgcaatccatcttgttcaatca |
| | tgcgaaacgatcctcatcctgtctcttgatcagatcttgatcccctgcgccatcagatccttggcg |
| | gcaagaaagccatccagtttactttgcagggcttcccaaccttaccagagggcgccccagctg |
| | gcaattccggttcgcttgctgtccataaaaccgcccagtctagctatcgccatgtaagcccactg |
| | caagctacctgctttctctttgcgcttgcgttttcccttgtccagatagcccagtagctgacattcat |
| | ccggggtcagcaccgtttctgcggactggctttctacgtgttccgcttcctttagcagcccttgcg |
| | ccatgcatttgttaaatcgaattatccattgctgttgacaaagggaatcaggggatcttgaagttcc |
| | tattccgaagttcctattctctagaaagtataggaacttcagagcgcttttgaagctaattcgagat |
| | catgcatgagctcactagtggatccccccgaccactcacaggattcctgcaagtaatgcgacat |
| | tggatactttgggattcctttatagtcagtgggttatccgaatttttgccttgttcgttgctacattgg |
| | ctaaatcagaacttcggcagctcgtcatcccaatacgcgtcaattcactggccgtcgttttactactt |
| | tccgctgcataaccctgcttcggggtcattatagcgattttttcggtatatccatcctttttcgcacg |
| | atatacaggattttgccaaagggttcgtgtagactttccttggtgtatccaacggcgtcagccgg |
| | gcaggataggtgaagtaggcccacccgcgagcgggtgttccttcttcactgtcccttattcgca |
| | cctggcggtgctcaacgggaatcctgctctgcgaggctggccgataagctctgataacgcagg |
| | aaagaacatgggcagttcaacctgttgatagtacgtactaagctctcatgtttcacgtactaagct |
| | ctcaagtttaacgtactaagctctcatgtttaacgaactaaaccctcatggctaacgtactaagctc |
| | tcatggctaacgtactaagctctcatgtttcacgtactaagctctcatgtttgaacaataaaattaat |
| | ataaatcagcaacttaaatagcctctaaggttttaagttttataagaaaaaaaagaatatataaggc |
| | ttttaaagcttttaaggtttaacggttgtggacaacaagccagggatgtaacgcactgagaagcc |
| | cttagagcctctcaaagcaattttgagtgacacaggaacacttaacggctgacaaccggctggt |
| | aacaggattagcagagcgaggtatgtaggcggtgctacaagattgtactgagagtgcaccata |
| | caggaaacagc (SEQ ID NO: 124) |
| pR6K- | gagagtgcaccatacaggaaacagctatgaccatgattacgccaagctcggcgcgccattgg |
| Donor(ZooTsy)_ | gattcggtcgaattcggactgggattctccaatattccttagcgcttccaataaagagcgcttttga |
| H1:12_ | agctgatgtgcttaaaaacttactcaatggaataattctagataattcactagtgattggaaatgttg |
| E1:105_E2:39_H2:12 | gatcctcatactcttcctttttcaattcagaagaactcgtcaagaaggcgatagaaggcgatgcg |
| | ctgcgaatcgggagcggcgataccgtaaagcacgaggaagcggtcagcccattcgccgcca |
| | agctcttcagcaatatcacgggtagccaacgctatgtcctgatagcggtccgccacacccagcc |
| | ggccacagtcgatgaatccagaaaagcggccattttccaccatgatattcggcaagcaggcat |
| | cgccatgggtcacgacgagatcctcgccgtcgggcatgcgcgccttgagcctggcgaacagt |
| | tcggctggcgcgagcccctgatgctcttcgtccagatcatcctgatcgacaagaccggcttcca |
| | tccgagtacgtgctcgctcgatgcgatgtttcgcttggtggtcgaatgggcaggtagccggatc |
| | aagcgtatgcagccgccgcattgcatcagccatgatggatactttctcggcaggagcaaggtg |
| | ggatgacaggagatcctgccccggcacttcgcccaatagcagccagtcccttcccgcttcagt |
| | gacaacgtcgagcacagctgcgcaaggaacgcccgtcgtggccagccacgatagccgcgct |
| | gcctcgtcctgcagttcattcagggcaccggacaggtcggtcttgacaaaaagaaccgggcgc |
| | ccctgcgctgacagccggaacacggcggcatcagagcagccgattgtctgttgtgcccagtca |
| | tagccgaatagcctctccacccaagcggccggagaacctgcgtgcaatccatcttgttcaatca |
| | tgcgaaacgatcctcatcctgtctcttgatcagatcttgatcccctgcgccatcagatccttggcg |
| | gcaagaaagccatccagtttactttgcagggcttcccaaccttaccagagggcgccccagctg |
| | gcaattccggttcgcttgctgtccataaaaccgcccagtctagctatcgccatgtaagcccactg |
| | caagctacctgctttctctttgcgcttgcgttttcccttgtccagatagcccagtagctgacattcat |
| | ccggggtcagcaccgtttctgcggactggctttctacgtgttccgcttcctttagcagcccttgcg |
| | ccatgcatttgttaaatcgaattatccattgctgttgacaaagggaatcaggggatcttgaagttcc |
| | tattccgaagttcctattctctagaaagtataggaacttcagagcgcttttgaagctaattcgagat |
| | catgcatgagctcactagtggatccccccgacattcctgcaagtaatgcgacattggatactttg |
| | ggattcctttatagtcagtgggttatccgaatttttgccttgttcgttgctacattggctaaatcagaa |
| | cttcggcagctcgtcatcccaatacgcgtcaattcactggccgtcgttttactactttccgctgcat |
| | aaccctgcttcggggtcattatagcgattttttcggtatatccatcctttttcgcacgatatacagga |
| | ttttgccaaagggttcgtgtagactttccttggtgtatccaacggcgtcagccgggcaggatagg |
| | tgaagtaggcccacccgcgagcgggtgttccttcttcactgtcccttattcgcacctggcggtgc |
| | tcaacgggaatcctgctctgcgaggctggccgataagctctgataacgcaggaaagaacatgg |
| | gcagttcaacctgttgatagtacgtactaagctctcatgtttcacgtactaagctctcaagtttaacg |
| | tactaagctctcatgtttaacgaactaaaccctcatggctaacgtactaagctctcatggctaacgt |
| | actaagctctcatgtttcacgtactaagctctcatgtttgaacaataaaattaatataaatcagcaac |
| | ttaaatagcctctaaggttttaagttttataagaaaaaaaagaatatataaggcttttaaagcttttaa |
| | ggtttaacggttgtggacaacaagccagggatgtaacgcactgagaagcccttagagcctctc |
| | aaagcaattttgagtgacacaggaacacttaacggctgacaaccggctggtaacaggattagc |
| | agagcgaggtatgtaggcggtgctacaagattgtactgagagtgcaccatacaggaaacagc |
| | (SEQ ID NO: 125) |
| pR6K- | gagagtgcaccatacaggaaacagctatgaccatgattacgccaagctcggcgcgccattgg |
| Donor(ZooTsy)_ | gattcggtcgaattcggactgggattctccaatattccttagcgcttccaataaagagcgcttttga |
| H1:12_ | agctgatgtgcttaaaaacttactcaatggaataattctagataattcactagtgattggaaatgttg |
| E1:95_E2:39_H2:12 | gatcctcatactcttcctttttcaattcagaagaactcgtcaagaaggcgatagaaggcgatgcg |
| | ctgcgaatcgggagcggcgataccgtaaagcacgaggaagcggtcagcccattcgccgcca |
| | agctcttcagcaatatcacgggtagccaacgctatgtcctgatagcggtccgccacacccagcc |
| | ggccacagtcgatgaatccagaaaagcggccattttccaccatgatattcggcaagcaggcat |
| | cgccatgggtcacgacgagatcctcgccgtcgggcatgcgcgccttgagcctggcgaacagt |
| | tcggctggcgcgagcccctgatgctcttcgtccagatcatcctgatcgacaagaccggcttcca |
| | tccgagtacgtgctcgctcgatgcgatgtttcgcttggtggtcgaatgggcaggtagccggatc |
| | aagcgtatgcagccgccgcattgcatcagccatgatggatactttctcggcaggagcaaggtg |
| | ggatgacaggagatcctgccccggcacttcgcccaatagcagccagtcccttcccgcttcagt |
| | gacaacgtcgagcacagctgcgcaaggaacgcccgtcgtggccagccacgatagccgcgct |
| | gcctcgtcctgcagttcattcagggcaccggacaggtcggtcttgacaaaaagaaccgggcgc |
| | ccctgcgctgacagccggaacacggcggcatcagagcagccgattgtctgttgtgcccagtca |
| | tagccgaatagcctctccacccaagcggccggagaacctgcgtgcaatccatcttgttcaatca |
| | tgcgaaacgatcctcatcctgtctcttgatcagatcttgatcccctgcgccatcagatccttggcg |
| | gcaagaaagccatccagtttactttgcagggcttcccaaccttaccagagggcgccccagctg |
| | gcaattccggttcgcttgctgtccataaaaccgcccagtctagctatcgccatgtaagcccactg |
| | caagctacctgctttctctttgcgcttgcgttttcccttgtccagatagcccagtagctgacattcat |
| | ccggggtcagcaccgtttctgcggactggctttctacgtgttccgcttcctttagcagcccttgcg |
| | ccatgcatttgttaaatcgaattatccattgctgttgacaaagggaatcaggggatcttgaagttcc |
| | tattccgaagttcctattctctagaaagtataggaacttcagagcgcttttgaagctaattcgagat |
| | catgcatgagctcactagtggatccccccgacgtaatgcgacattggatactttgggattccttta |
| | tagtcagtgggttatccgaatttttgccttgttcgttgctacattggctaaatcagaacttcggcagc |
| | tcgtcatcccaatacgcgtcaattcactggccgtcgttttactactttccgctgcataaccctgcttc |
| | ggggtcattatagcgattttttcggtatatccatcctttttcgcacgatatacaggattttgccaaag |
| | ggttcgtgtagactttccttggtgtatccaacggcgtcagccgggcaggataggtgaagtaggc |
| | ccacccgcgagcgggtgttccttcttcactgtcccttattcgcacctggcggtgctcaacgggaa |
| | tcctgctctgcgaggctggccgataagctctgataacgcaggaaagaacatgggcagttcaac |
| | ctgttgatagtacgtactaagctctcatgtttcacgtactaagctctcaagtttaacgtactaagctc |
| | tcatgtttaacgaactaaaccctcatggctaacgtactaagctctcatggctaacgtactaagctct |
| | catgtttcacgtactaagctctcatgtttgaacaataaaattaatataaatcagcaacttaaatagcc |
| | tctaaggttttaagttttataagaaaaaaaagaatatataaggcttttaaagcttttaaggtttaacgg |
| | ttgtggacaacaagccagggatgtaacgcactgagaagcccttagagcctctcaaagcaatttt |
| | gagtgacacaggaacacttaacggctgacaaccggctggtaacaggattagcagagcgaggt |
| | atgtaggcggtgctacaagattgtactgagagtgcaccatacaggaaacagc (SEQ ID |
| | NO: 126) |
| pR6K- | gagagtgcaccatacaggaaacagctatgaccatgattacgccaagctcggcgcgccattgg |
| Donor(ZooTsy)_ | gattcggtcgaattcggactgggattctccaatattccttagcgcagagcgcttttgaagctgatg |
| H1:12_ | tgcttaaaaacttactcaatggaataattctagataattcactagtgattggaaatgttggatcctca |
| E1:135_E2:30_H2:12 | tactcttcctttttcaattcagaagaactcgtcaagaaggcgatagaaggcgatgcgctgcgaat |
| | cgggagcggcgataccgtaaagcacgaggaagcggtcagcccattcgccgccaagctcttc |
| | agcaatatcacgggtagccaacgctatgtcctgatagcggtccgccacacccagccggccac |
| | agtcgatgaatccagaaaagcggccattttccaccatgatattcggcaagcaggcatcgccatg |
| | ggtcacgacgagatcctcgccgtcgggcatgcgcgccttgagcctggcgaacagttcggctg |
| | gcgcgagcccctgatgctcttcgtccagatcatcctgatcgacaagaccggcttccatccgagt |
| | acgtgctcgctcgatgcgatgtttcgcttggtggtcgaatgggcaggtagccggatcaagcgta |
| | tgcagccgccgcattgcatcagccatgatggatactttctcggcaggagcaaggtgggatgac |
| | aggagatcctgccccggcacttcgcccaatagcagccagtcccttcccgcttcagtgacaacgt |
| | cgagcacagctgcgcaaggaacgcccgtcgtggccagccacgatagccgcgctgcctcgtc |
| | ctgcagttcattcagggcaccggacaggtcggtcttgacaaaaagaaccgggcgcccctgcg |
| | ctgacagccggaacacggcggcatcagagcagccgattgtctgttgtgcccagtcatagccga |
| | atagcctctccacccaagcggccggagaacctgcgtgcaatccatcttgttcaatcatgcgaaa |
| | cgatcctcatcctgtctcttgatcagatcttgatcccctgcgccatcagatccttggggcaagaa |
| | agccatccagtttactttgcagggcttcccaaccttaccagagggcgccccagctggcaattcc |
| | ggttcgcttgctgtccataaaaccgcccagtctagctatcgccatgtaagcccactgcaagctac |
| | ctgctttctctttgcgcttgcgttttcccttgtccagatagcccagtagctgacattcatccggggtc |
| | agcaccgtttctgcggactggctttctacgtgttccgcttcctttagcagcccttgcgccatgcatt |
| | tgttaaatcgaattatccattgctgttgacaaagggaatcaggggatcttgaagttcctattccgaa |
| | gttcctattctctagaaagtataggaacttcagagcgcttttgaagctaattcgagatcatgcatga |
| | gctcactagtggatccccccgactacttctaatgctgtgacatcactcacaggattcctgcaagta |
| | atgcgacattggatactttgggattcctttatagtcagtgggttatccgaatttttgccttgttcgttg |
| | ctacattggctaaatcagaacttcggcagctcgtcatcccaatacgcgtcaattcactggccgtc |
| | gttttactactttccgctgcataaccctgcttcggggtcattatagcgattttttcggtatatccatcc |
| | tttttcgcacgatatacaggattttgccaaagggttcgtgtagactttccttggtgtatccaacggcg |
| | tcagccgggcaggataggtgaagtaggcccacccgcgagcgggtgttccttcttcactgtccc |
| | ttattcgcacctggcggtgctcaacgggaatcctgctctgcgaggctggccgataagctctgat |
| | aacgcaggaaagaacatgggcagttcaacctgttgatagtacgtactaagctctcatgtttcacg |
| | tactaagctctcaagtttaacgtactaagctctcatgtttaacgaactaaaccctcatggctaacgt |
| | actaagctctcatggctaacgtactaagctctcatgtttcacgtactaagctctcatgtttgaacaat |
| | aaaattaatataaatcagcaacttaaatagcctctaaggttttaagttttataagaaaaaaaagaat |
| | atataaggcttttaaagcttttaaggtttaacggttgtggacaacaagccagggatgtaacgcact |
| | gagaagcccttagagcctctcaaagcaattttgagtgacacaggaacacttaacggctgacaac |
| | cggctggtaacaggattagcagagcgaggtatgtaggcggtgctacaagattgtactgagagt |
| | gcaccatacaggaaacagc (SEQ ID NO: 127) |
| pR6K- | gagagtgcaccatacaggaaacagctatgaccatgattacgccaagctcggcgcgccattgg |
| Donor(ZooTsy)_ | gattcggtcgaattcggactgggattctccaatattccttagagcgcttttgaagctgatgtgctta |
| H1:12_ | aaaacttactcaatggaataattctagataattcactagtgattggaaatgttggatcctcatactctt |
| E1:135_E2:25_H2:12 | cctttttcaattcagaagaactcgtcaagaaggcgatagaaggcgatgcgctgcgaatcggga |
| | gcggcgataccgtaaagcacgaggaagcggtcagcccattcgccgccaagctcttcagcaat |
| | atcacgggtagccaacgctatgtcctgatagcggtccgccacacccagccggccacagtcgat |
| | gaatccagaaaagcggccattttccaccatgatattcggcaagcaggcatcgccatgggtcac |
| | gacgagatcctcgccgtcgggcatgcgcgccttgagcctggcgaacagttcggctggcgcga |
| | gcccctgatgctcttcgtccagatcatcctgatcgacaagaccggcttccatccgagtacgtgct |
| | cgctcgatgcgatgtttcgcttggtggtcgaatgggcaggtagccggatcaagcgtatgcagc |
| | cgccgcattgcatcagccatgatggatactttctcggcaggagcaaggtgggatgacaggaga |
| | tcctgccccggcacttcgcccaatagcagccagtcccttcccgcttcagtgacaacgtcgagca |
| | cagctgcgcaaggaacgcccgtcgtggccagccacgatagccgcgctgcctcgtcctgcagt |
| | tcattcagggcaccggacaggtcggtcttgacaaaaagaaccgggcgcccctgcgctgacag |
| | ccggaacacggcggcatcagagcagccgattgtctgttgtgcccagtcatagccgaatagcct |
| | ctccacccaagcggccggagaacctgcgtgcaatccatcttgttcaatcatgcgaaacgatcct |
| | catcctgtctcttgatcagatcttgatcccctgcgccatcagatccttggcggcaagaaagccatc |
| | cagtttactttgcagggcttcccaaccttaccagagggcgccccagctggcaattccggttcgct |
| | tgctgtccataaaaccgcccagtctagctatcgccatgtaagcccactgcaagctacctgctttct |
| | ctttgcgcttgcgttttcccttgtccagatagcccagtagctgacattcatccggggtcagcaccg |
| | tttctgcggactggctttctacgtgttccgcttcctttagcagcccttgcgccatgcatttgttaaatc |
| | gaattatccattgctgttgacaaagggaatcaggggatcttgaagttcctattccgaagttcctatt |
| | ctctagaaagtataggaacttcagagcgcttttgaagctaattcgagatcatgcatgagctcacta |
| | gtggatccccccgactacttctaatgctgtgacatcactcacaggattcctgcaagtaatgcgac |
| | attggatactttgggattcctttatagtcagtgggttatccgaatttttgccttgttcgttgctacatt |
| | ggctaaatcagaacttcggcagctcgtcatcccaatacgcgtcaattcactggccgtcgttttacta |
| | ctttccgctgcataaccctgcttcggggtcattatagcgattttttcggtatatccatcctttttcgca |
| | cgatatacaggattttgccaaagggttcgtgtagactttccttggtgtatccaacggcgtcagccg |
| | ggcaggataggtgaagtaggcccacccgcgagcgggtgttccttcttcactgtcccttattcgc |
| | acctggcggtgctcaacgggaatcctgctctgcgaggctggccgataagctctgataacgcag |
| | gaaagaacatgggcagttcaacctgttgatagtacgtactaagctctcatgtttcacgtactaagc |
| | tctcaagtttaacgtactaagctctcatgtttaacgaactaaaccctcatggctaacgtactaagct |
| | ctcatggctaacgtactaagctctcatgtttcacgtactaagctctcatgtttgaacaataaaattaa |
| | tataaatcagcaacttaaatagcctctaaggttttaagttttataagaaaaaaaagaatatataagg |
| | cttttaaagcttttaaggtttaacggttgtggacaacaagccagggatgtaacgcactgagaagc |
| | ccttagagcctctcaaagcaattttgagtgacacaggaacacttaacggctgacaaccggctgg |
| | taacaggattagcagagcgaggtatgtaggcggtgctacaagattgtactgagagtgcaccat |
| | acaggaaacagc (SEQ ID NO: 128) |
| pR6K- | gagagtgcaccatacaggaaacagctatgaccatgattacgccaagctcggcgcgccattgg |
| Donor(ZooTsy)_ | gattcggtcgaattcggactgggattctccaatatagagcgcttttgaagctgatgtgcttaaaaa |
| H1:12_ | cttactcaatggaataattctagataattcactagtgattggaaatgttggatcctcatactcttcctt |
| E1:135_E2:20_H2:12t | ttcaattcagaagaactcgtcaagaaggcgatagaaggcgatgcgctgcgaatcgggagcgg |
| | cgataccgtaaagcacgaggaagcggtcagcccattcgccgccaagctcttcagcaatatcac |
| | gggtagccaacgctatgtcctgatagcggtccgccacacccagccggccacagtcgatgaat |
| | ccagaaaagcggccattttccaccatgatattcggcaagcaggcatcgccatgggtcacgacg |
| | agatcctcgccgtcgggcatgcgcgccttgagcctggcgaacagttcggctggcgcgagccc |
| | ctgatgctcttcgtccagatcatcctgatcgacaagaccggcttccatccgagtacgtgctcgct |
| | cgatgcgatgtttcgcttggtggtcgaatgggcaggtagccggatcaagcgtatgcagccgcc |
| | gcattgcatcagccatgatggatactttctcggcaggagcaaggtgggatgacaggagatcct |
| | gccccggcacttcgcccaatagcagccagtcccttcccgcttcagtgacaacgtcgagcacag |
| | ctgcgcaaggaacgcccgtcgtggccagccacgatagccgcgctgcctcgtcctgcagttcat |
| | tcagggcaccggacaggtcggtcttgacaaaaagaaccgggcgcccctgcgctgacagccg |
| | gaacacggcggcatcagagcagccgattgtctgttgtgcccagtcatagccgaatagcctctcc |
| | acccaagcggccggagaacctgcgtgcaatccatcttgttcaatcatgcgaaacgatcctcatc |
| | ctgtctcttgatcagatcttgatcccctgcgccatcagatccttggcggcaagaaagccatccag |
| | tttactttgcagggcttcccaaccttaccagagggcgccccagctggcaattccggttcgcttgct |
| | gtccataaaaccgcccagtctagctatcgccatgtaagcccactgcaagctacctgctttctcttt |
| | gcgcttgcgttttcccttgtccagatagcccagtagctgacattcatccggggtcagcaccgtttc |
| | tgcggactggctttctacgtgttccgcttcctttagcagcccttgcgccatgcatttgttaaatcga |
| | attatccattgctgttgacaaagggaatcaggggatcttgaagttcctattccgaagttcctattctc |
| | tagaaagtataggaacttcagagcgcttttgaagctaattcgagatcatgcatgagctcactagtg |
| | gatccccccgactacttctaatgctgtgacatcactcacaggattcctgcaagtaatgcgacattg |
| | gatactttgggattcctttatagtcagtgggttatccgaatttttgccttgttcgttgctacattggct |
| | aaatcagaacttcggcagctcgtcatcccaatacgcgtcaattcactggccgtcgttttactactttc |
| | cgctgcataaccctgcttcggggtcattatagcgattttttcggtatatccatcctttttcgcacgat |
| | atacaggattttgccaaagggttcgtgtagactttccttggtgtatccaacggcgtcagccgggc |
| | aggataggtgaagtaggcccacccgcgagcgggtgttccttcttcactgtcccttattcgcacct |
| | ggcggtgctcaacgggaatcctgctctgcgaggctggccgataagctctgataacgcaggaa |
| | agaacatgggcagttcaacctgttgatagtacgtactaagctctcatgtttcacgtactaagctctc |
| | aagtttaacgtactaagctctcatgtttaacgaactaaaccctcatggctaacgtactaagctctca |
| | tggctaacgtactaagctctcatgtttcacgtactaagctctcatgtttgaacaataaaattaatata |
| | aatcagcaacttaaatagcctctaaggttttaagttttataagaaaaaaaagaatatataaggctttt |
| | aaagcttttaaggtttaacggttgtggacaacaagccagggatgtaacgcactgagaagccctt |
| | agagcctctcaaagcaattttgagtgacacaggaacacttaacggctgacaaccggctggtaa |
| | caggattagcagagcgaggtatgtaggcggtgctacaagattgtactgagagtgcaccataca |
| | ggaaacagc (SEQ ID NO: 129) |
| pR6K- | gagagtgcaccatacaggaaacagctatgaccatgattacgccaagctcggcgcgccattgg |
| Donor(ZooTsy)_ | gattcggtcgaattcggactgggattctccagagcgcttttgaagctgatgtgcttaaaaacttac |
| H1:12_ | tcaatggaataattctagataattcactagtgattggaaatgttggatcctcatactcttcctttttca |
| E1:135_E2:15_H2:12 | attcagaagaactcgtcaagaaggcgatagaaggcgatgcgctgcgaatcgggagcggcgat |
| | accgtaaagcacgaggaagcggtcagcccattcgccgccaagctcttcagcaatatcacgggt |
| | agccaacgctatgtcctgatagcggtccgccacacccagccggccacagtcgatgaatccag |
| | aaaagcggccattttccaccatgatattcggcaagcaggcatcgccatgggtcacgacgagat |
| | cctcgccgtcgggcatgcgcgccttgagcctggcgaacagttcggctggcgcgagcccctga |
| | tgctcttcgtccagatcatcctgatcgacaagaccggcttccatccgagtacgtgctcgctcgat |
| | gcgatgtttcgcttggtggtcgaatgggcaggtagccggatcaagcgtatgcagccgccgcat |
| | tgcatcagccatgatggatactttctcggcaggagcaaggtgggatgacaggagatcctgccc |
| | cggcacttcgcccaatagcagccagtcccttcccgcttcagtgacaacgtcgagcacagctgc |
| | gcaaggaacgcccgtcgtggccagccacgatagccgcgctgcctcgtcctgcagttcattcag |
| | ggcaccggacaggtcggtcttgacaaaaagaaccgggcgcccctgcgctgacagccggaac |
| | acggcggcatcagagcagccgattgtctgttgtgcccagtcatagccgaatagcctctccaccc |
| | aagcggccggagaacctgcgtgcaatccatcttgttcaatcatgcgaaacgatcctcatcctgtc |
| | tcttgatcagatcttgatcccctgcgccatcagatccttggcggcaagaaagccatccagtttact |
| | ttgcagggcttcccaaccttaccagagggcgccccagctggcaattccggttcgcttgctgtcc |
| | ataaaaccgcccagtctagctatcgccatgtaagcccactgcaagctacctgctttctctttgcgc |
| | ttgcgttttcccttgtccagatagcccagtagctgacattcatccggggtcagcaccgtttctgcg |
| | gactggctttctacgtgttccgcttcctttagcagcccttgcgccatgcatttgttaaatcgaattat |
| | ccattgctgttgacaaagggaatcaggggatcttgaagttcctattccgaagttcctattctctaga |
| | aagtataggaacttcagagcgcttttgaagctaattcgagatcatgcatgagctcactagtggatc |
| | cccccgactacttctaatgctgtgacatcactcacaggattcctgcaagtaatgcgacattggata |
| | ctttgggattcctttatagtcagtgggttatccgaatttttgccttgttcgttgctacattggctaaat |
| | cagaacttcggcagctcgtcatcccaatacgcgtcaattcactggccgtcgttttactactttccgct |
| | gcataaccctgcttcggggtcattatagcgattttttcggtatatccatcctttttcgcacgatataca |
| | ggattttgccaaagggttcgtgtagactttccttggtgtatccaacggcgtcagccgggcaggat |
| | aggtgaagtaggcccacccgcgagcgggtgttccttcttcactgtcccttattcgcacctggcg |
| | gtgctcaacgggaatcctgctctgcgaggctggccgataagctctgataacgcaggaaagaac |
| | atgggcagttcaacctgttgatagtacgtactaagctctcatgtttcacgtactaagctctcaagttt |
| | aacgtactaagctctcatgtttaacgaactaaaccctcatggctaacgtactaagctctcatggct |
| | aacgtactaagctctcatgtttcacgtactaagctctcatgtttgaacaataaaattaatataaatca |
| | gcaacttaaatagcctctaaggttttaagttttataagaaaaaaaagaatatataaggcttttaaag |
| | cttttaaggtttaacggttgtggacaacaagccagggatgtaacgcactgagaagcccttagag |
| | cctctcaaagcaattttgagtgacacaggaacacttaacggctgacaaccggctggtaacagg |
| | attagcagagcgaggtatgtaggcggtgctacaagattgtactgagagtgcaccatacaggaa |
| | acagc (SEQ ID NO: 130) |
| pR6K- | gagagtgcaccatacaggaaacagctatgaccatgattacgccaagctcggcgcgccattgg |
| Donor(ZooTsy)_ | gattcggtcgaattcggactgggatagagcgcttttgaagctgatgtgcttaaaaacttactcaat |
| H1:12_ | ggaataattctagataattcactagtgattggaaatgttggatcctcatactcttcctttttcaattca |
| E1:135_E2:10_H2:12 | gaagaactcgtcaagaaggcgatagaaggcgatgcgctgcgaatcgggagcggcgataccg |
| | taaagcacgaggaagcggtcagcccattcgccgccaagctcttcagcaatatcacgggtagcc |
| | aacgctatgtcctgatagcggtccgccacacccagccggccacagtcgatgaatccagaaaa |
| | gcggccattttccaccatgatattcggcaagcaggcatcgccatgggtcacgacgagatcctcg |
| | ccgtcgggcatgcgcgccttgagcctggcgaacagttcggctggcgcgagcccctgatgctc |
| | ttcgtccagatcatcctgatcgacaagaccggcttccatccgagtacgtgctcgctcgatgcgat |
| | gtttcgcttggtggtcgaatgggcaggtagccggatcaagcgtatgcagccgccgcattgcatc |
| | agccatgatggatactttctcggcaggagcaaggtgggatgacaggagatcctgccccggca |
| | cttcgcccaatagcagccagtcccttcccgcttcagtgacaacgtcgagcacagctgcgcaag |
| | gaacgcccgtcgtggccagccacgatagccgcgctgcctcgtcctgcagttcattcagggcac |
| | cggacaggtcggtcttgacaaaaagaaccgggcgcccctgcgctgacagccggaacacggc |
| | ggcatcagagcagccgattgtctgttgtgcccagtcatagccgaatagcctctccacccaagcg |
| | gccggagaacctgcgtgcaatccatcttgttcaatcatgcgaaacgatcctcatcctgtctcttga |
| | tcagatcttgatcccctgcgccatcagatccttggcggcaagaaagccatccagtttactttgca |
| | gggcttcccaaccttaccagagggcgccccagctggcaattccggttcgcttgctgtccataaa |
| | accgcccagtctagctatcgccatgtaagcccactgcaagctacctgctttctctttgcgcttgcg |
| | ttttcccttgtccagatagcccagtagctgacattcatccggggtcagcaccgtttctgcggactg |
| | gctttctacgtgttccgcttcctttagcagcccttgcgccatgcatttgttaaatcgaattatccattg |
| | ctgttgacaaagggaatcaggggatcttgaagttcctattccgaagttcctattctctagaaagtat |
| | aggaacttcagagcgcttttgaagctaattcgagatcatgcatgagctcactagtggatcccccc |
| | gactacttctaatgctgtgacatcactcacaggattcctgcaagtaatgcgacattggatactttgg |
| | gattcctttatagtcagtgggttatccgaatttttgccttgttcgttgctacattggctaaatcagaac |
| | ttcggcagctcgtcatcccaatacgcgtcaattcactggccgtcgttttactactttccgctgcata |
| | accctgcttcggggtcattatagcgattttttcggtatatccatcctttttcgcacgatatacaggatt |
| | ttgccaaagggttcgtgtagactttccttggtgtatccaacggcgtcagccgggcaggataggt |
| | gaagtaggcccacccgcgagcgggtgttccttcttcactgtcccttattcgcacctggcggtgct |
| | caacgggaatcctgctctgcgaggctggccgataagctctgataacgcaggaaagaacatgg |
| | gcagttcaacctgttgatagtacgtactaagctctcatgtttcacgtactaagctctcaagtttaacg |
| | tactaagctctcatgtttaacgaactaaaccctcatggctaacgtactaagctctcatggctaacgt |
| | actaagctctcatgtttcacgtactaagctctcatgtttgaacaataaaattaatataaatcagcaac |
| | ttaaatagcctctaaggttttaagttttataagaaaaaaaagaatatataaggcttttaaagcttttaa |
| | ggtttaacggttgtggacaacaagccagggatgtaacgcactgagaagcccttagagcctctc |
| | aaagcaattttgagtgacacaggaacacttaacggctgacaaccggctggtaacaggattagc |
| | agagcgaggtatgtaggcggtgctacaagattgtactgagagtgcaccatacaggaaacagc |
| | (SEQ ID NO: 131) |
| pR6K- | gagagtgcaccatacaggaaacagctatgaccatgattacgccaagctcggcgcgccattgg |
| Donor(ZooTsy)_ | gattcggtcgaattcagagcgcttttgaagctgatgtgcttaaaaacttactcaatggaataattct |
| H1:12_ | agataattcactagtgattggaaatgttggatcctcatactcttcctttttcaattcagaagaactcgt |
| E1:135_E2:0_H2:12 | caagaaggcgatagaaggcgatgcgctgcgaatcgggagcggcgataccgtaaagcacga |
| | ggaagcggtcagcccattcgccgccaagctcttcagcaatatcacgggtagccaacgctatgt |
| | cctgatagcggtccgccacacccagccggccacagtcgatgaatccagaaaagcggccatttt |
| | ccaccatgatattcggcaagcaggcatcgccatgggtcacgacgagatcctcgccgtcgggc |
| | atgcgcgccttgagcctggcgaacagttcggctggcgcgagcccctgatgctcttcgtccaga |
| | tcatcctgatcgacaagaccggcttccatccgagtacgtgctcgctcgatgcgatgtttcgcttg |
| | gtggtcgaatgggcaggtagccggatcaagcgtatgcagccgccgcattgcatcagccatgat |
| | ggatactttctcggcaggagcaaggtgggatgacaggagatcctgccccggcacttcgccca |
| | atagcagccagtcccttcccgcttcagtgacaacgtcgagcacagctgcgcaaggaacgccc |
| | gtcgtggccagccacgatagccgcgctgcctcgtcctgcagttcattcagggcaccggacag |
| | gtcggtcttgacaaaaagaaccgggcgcccctgcgctgacagccggaacacggcggcatca |
| | gagcagccgattgtctgttgtgcccagtcatagccgaatagcctctccacccaagcggccgga |
| | gaacctgcgtgcaatccatcttgttcaatcatgcgaaacgatcctcatcctgtctcttgatcagatc |
| | ttgatcccctgcgccatcagatccttggcggcaagaaagccatccagtttactttgcagggcttc |
| | ccaaccttaccagagggcgccccagctggcaattccggttcgcttgctgtccataaaaccgccc |
| | agtctagctatcgccatgtaagcccactgcaagctacctgctttctctttgcgcttgcgttttccctt |
| | gtccagatagcccagtagctgacattcatccggggtcagcaccgtttctgcggactggctttcta |
| | cgtgttccgcttcctttagcagcccttgcgccatgcatttgttaaatcgaattatccattgctgttga |
| | caaagggaatcaggggatcttgaagttcctattccgaagttcctattctctagaaagtataggaac |
| | ttcagagcgcttttgaagctaattcgagatcatgcatgagctcactagtggatccccccgactact |
| | tctaatgctgtgacatcactcacaggattcctgcaagtaatgcgacattggatactttgggattcct |
| | ttatagtcagtgggttatccgaatttttgccttgttcgttgctacattggctaaatcagaacttcggca |
| | gctcgtcatcccaatacgcgtcaattcactggccgtcgttttactactttccgctgcataaccctgc |
| | ttcggggtcattatagcgattttttcggtatatccatcctttttcgcacgatatacaggattttgccaa |
| | agggttcgtgtagactttccttggtgtatccaacggcgtcagccgggcaggataggtgaagtag |
| | gcccacccgcgagcgggtgttccttcttcactgtcccttattcgcacctggcggtgctcaacgg |
| | gaatcctgctctgcgaggctggccgataagctctgataacgcaggaaagaacatgggcagttc |
| | aacctgttgatagtacgtactaagctctcatgtttcacgtactaagctctcaagtttaacgtactaag |
| | ctctcatgtttaacgaactaaaccctcatggctaacgtactaagctctcatggctaacgtactaag |
| | ctctcatgtttcacgtactaagctctcatgtttgaacaataaaattaatataaatcagcaacttaaata |
| | gcctctaaggttttaagttttataagaaaaaaaagaatatataaggcttttaaagcttttaaggtttaa |
| | cggttgtggacaacaagccagggatgtaacgcactgagaagcccttagagcctctcaaagca |
| | attttgagtgacacaggaacacttaacggctgacaaccggctggtaacaggattagcagagcg |
| | aggtatgtaggcggtgctacaagattgtactgagagtgcaccatacaggaaacagc (SEQ |
| | ID NO: 132) |
| pR6K- | gagagtgcaccatacaggaaacagctatgaccatgattacgccaagctcggcgcgccattgg |
| Donor(ZooTsy)_ | gattcggtcgaattcggactgggattctccaatatagagcgcttttgaagctgatgtgcttaaaaa |
| H1:12_ | cttactcaatggaataattctagataattcactagtgattggaaatgttggatcctcatactcttcctt |
| E1:0_E2:20_H2:12 | tttcaattcagaagaactcgtcaagaaggcgatagaaggcgatgcgctgcgaatcgggagcgg |
| | cgataccgtaaagcacgaggaagcggtcagcccattcgccgccaagctcttcagcaatatcac |
| | gggtagccaacgctatgtcctgatagcggtccgccacacccagccggccacagtcgatgaat |
| | ccagaaaagcggccattttccaccatgatattcggcaagcaggcatcgccatgggtcacgacg |
| | agatcctcgccgtcgggcatgcgcgccttgagcctggcgaacagttcggctggcgcgagccc |
| | ctgatgctcttcgtccagatcatcctgatcgacaagaccggcttccatccgagtacgtgctcgct |
| | cgatgcgatgtttcgcttggtggtcgaatgggcaggtagccggatcaagcgtatgcagccgcc |
| | gcattgcatcagccatgatggatactttctcggcaggagcaaggtgggatgacaggagatcct |
| | gccccggcacttcgcccaatagcagccagtcccttcccgcttcagtgacaacgtcgagcacag |
| | ctgcgcaaggaacgcccgtcgtggccagccacgatagccgcgctgcctcgtcctgcagttcat |
| | tcagggcaccggacaggtcggtcttgacaaaaagaaccgggcgcccctgcgctgacagccg |
| | gaacacggcggcatcagagcagccgattgtctgttgtgcccagtcatagccgaatagcctctcc |
| | acccaagcggccggagaacctgcgtgcaatccatcttgttcaatcatgcgaaacgatcctcatc |
| | ctgtctcttgatcagatcttgatcccctgcgccatcagatccttggcggcaagaaagccatccag |
| | tttactttgcagggcttcccaaccttaccagagggcgccccagctggcaattccggttcgcttgct |
| | gtccataaaaccgcccagtctagctatcgccatgtaagcccactgcaagctacctgctttctcttt |
| | gcgcttgcgttttcccttgtccagatagcccagtagctgacattcatccggggtcagcaccgtttc |
| | tgcggactggctttctacgtgttccgcttcctttagcagcccttgcgccatgcatttgttaaatcga |
| | attatccattgctgttgacaaagggaatcaggggatcttgaagttcctattccgaagttcctattctc |
| | tagaaagtataggaacttcagagcgcttttgaagctaattcgagatcatgcatgagctcactagtg |
| | gatccccccgaccggcagctcgtcatcccaatacgcgtcaattcactggccgtcgttttactactt |
| | tccgctgcataaccctgcttcggggtcattatagcgattttttcggtatatccatcctttttcgcacg |
| | atatacaggattttgccaaagggttcgtgtagactttccttggtgtatccaacggcgtcagccgg |
| | gcaggataggtgaagtaggcccacccgcgagcgggtgttccttcttcactgtcccttattcgca |
| | cctggcggtgctcaacgggaatcctgctctgcgaggctggccgataagctctgataacgcagg |
| | aaagaacatgggcagttcaacctgttgatagtacgtactaagctctcatgtttcacgtactaagct |
| | ctcaagtttaacgtactaagctctcatgtttaacgaactaaaccctcatggctaacgtactaagctc |
| | tcatggctaacgtactaagctctcatgtttcacgtactaagctctcatgtttgaacaataaaattaat |
| | ataaatcagcaacttaaatagcctctaaggttttaagttttataagaaaaaaaagaatatataaggc |
| | ttttaaagcttttaaggtttaacggttgtggacaacaagccagggatgtaacgcactgagaagcc |
| | cttagagcctctcaaagcaattttgagtgacacaggaacacttaacggctgacaaccggctggt |
| | aacaggattagcagagcgaggtatgtaggcggtgctacaagattgtactgagagtgcaccata |
| | caggaaacagc (SEQ ID NO: 133) |
| pR6K- | gagagtgcaccatacaggaaacagctatgaccatgattacgccaagctcggcgcgccattgg |
| Donor(ZooTsy)_H1:0_ | gattcggtcgaattcggactgggattctccaatatagagcgcttttgaagctgatgtgcttaaaaa |
| E1:135_E2:20_H2:12 | cttactcaatggaataattctagataattcactagtgattggaaatgttggatcctcatactcttcctt |
| | tttcaattcagaagaactcgtcaagaaggcgatagaaggcgatgcgctgcgaatcgggagcgg |
| | cgataccgtaaagcacgaggaagcggtcagcccattcgccgccaagctcttcagcaatatcac |
| | gggtagccaacgctatgtcctgatagcggtccgccacacccagccggccacagtcgatgaat |
| | ccagaaaagcggccattttccaccatgatattcggcaagcaggcatcgccatgggtcacgacg |
| | agatcctcgccgtcgggcatgcgcgccttgagcctggcgaacagttcggctggcgcgagccc |
| | ctgatgctcttcgtccagatcatcctgatcgacaagaccggcttccatccgagtacgtgctcgct |
| | cgatgcgatgtttcgcttggtggtcgaatgggcaggtagccggatcaagcgtatgcagccgcc |
| | gcattgcatcagccatgatggatactttctcggcaggagcaaggtgggatgacaggagatcct |
| | gccccggcacttcgcccaatagcagccagtcccttcccgcttcagtgacaacgtcgagcacag |
| | ctgcgcaaggaacgcccgtcgtggccagccacgatagccgcgctgcctcgtcctgcagttcat |
| | tcagggcaccggacaggtcggtcttgacaaaaagaaccgggcgcccctgcgctgacagccg |
| | gaacacggcggcatcagagcagccgattgtctgttgtgcccagtcatagccgaatagcctctcc |
| | acccaagcggccggagaacctgcgtgcaatccatcttgttcaatcatgcgaaacgatcctcatc |
| | ctgtctcttgatcagatcttgatcccctgcgccatcagatccttggcggcaagaaagccatccag |
| | tttactttgcagggcttcccaaccttaccagagggcgccccagctggcaattccggttcgcttgct |
| | gtccataaaaccgcccagtctagctatcgccatgtaagcccactgcaagctacctgctttctcttt |
| | gcgcttgcgttttcccttgtccagatagcccagtagctgacattcatccggggtcagcaccgtttc |
| | tgcggactggctttctacgtgttccgcttcctttagcagcccttgcgccatgcatttgttaaatcga |
| | attatccattgctgttgacaaagggaatcaggggatcttgaagttcctattccgaagttcctattctc |
| | tagaaagtataggaacttcagagcgcttttgaagctaattcgagatcatgcatgagctcactagtg |
| | gatccccccgactacttctaatgctgtgacatcactcacaggattcctgcaagtaatgcgacattg |
| | gatactttgggattcctttatagtcagtgggttatccgaatttttgccttgttcgttgctacattggct |
| | aaatcagaacttatcccaatacgcgtcaattcactggccgtcgttttactactttccgctgcataacc |
| | ctgcttcggggtcattatagcgattttttcggtatatccatcctttttcgcacgatatacaggattttg |
| | ccaaagggttcgtgtagactttccttggtgtatccaacggcgtcagccgggcaggataggtgaa |
| | gtaggcccacccgcgagcgggtgttccttcttcactgtcccttattcgcacctggcggtgctcaa |
| | cgggaatcctgctctgcgaggctggccgataagctctgataacgcaggaaagaacatgggca |
| | gttcaacctgttgatagtacgtactaagctctcatgtttcacgtactaagctctcaagtttaacgtac |
| | taagctctcatgtttaacgaactaaaccctcatggctaacgtactaagctctcatggctaacgtact |
| | aagctctcatgtttcacgtactaagctctcatgtttgaacaataaaattaatataaatcagcaactta |
| | aatagcctctaaggttttaagttttataagaaaaaaaagaatatataaggcttttaaagcttttaagg |
| | tttaacggttgtggacaacaagccagggatgtaacgcactgagaagcccttagagcctctcaaa |
| | gcaattttgagtgacacaggaacacttaacggctgacaaccggctggtaacaggattagcaga |
| | gcgaggtatgtaggcggtgctacaagattgtactgagagtgcaccatacaggaaacagc |
| | (SEQ ID NO: 134) |
| pR6K- | gagagtgcaccatacaggaaacagctatgaccatgattacgccaagctcggcgcgccattgg |
| Donor(ZooTsy)_H1:0_ | gattcggtcgaattcggactgggattctccaatatagagcgcttttgaagctgatgtgcttaaaaa |
| E1:0_E2:20_H2:12 | cttactcaatggaataattctagataattcactagtgattggaaatgttggatcctcatactcttcctt |
| | tttcaattcagaagaactcgtcaagaaggcgatagaaggcgatgcgctgcgaatcgggagcgg |
| | cgataccgtaaagcacgaggaagcggtcagcccattcgccgccaagctcttcagcaatatcac |
| | gggtagccaacgctatgtcctgatagcggtccgccacacccagccggccacagtcgatgaat |
| | ccagaaaagcggccattttccaccatgatattcggcaagcaggcatcgccatgggtcacgacg |
| | agatcctcgccgtcgggcatgcgcgccttgagcctggcgaacagttcggctggcgcgagccc |
| | ctgatgctcttcgtccagatcatcctgatcgacaagaccggcttccatccgagtacgtgctcgct |
| | cgatgcgatgtttcgcttggtggtcgaatgggcaggtagccggatcaagcgtatgcagccgcc |
| | gcattgcatcagccatgatggatactttctcggcaggagcaaggtgggatgacaggagatcct |
| | gccccggcacttcgcccaatagcagccagtcccttcccgcttcagtgacaacgtcgagcacag |
| | ctgcgcaaggaacgcccgtcgtggccagccacgatagccgcgctgcctcgtcctgcagttcat |
| | tcagggcaccggacaggtcggtcttgacaaaaagaaccgggcgcccctgcgctgacagccg |
| | gaacacggcggcatcagagcagccgattgtctgttgtgcccagtcatagccgaatagcctctcc |
| | acccaagcggccggagaacctgcgtgcaatccatcttgttcaatcatgcgaaacgatcctcatc |
| | ctgtctcttgatcagatcttgatcccctgcgccatcagatccttggcggcaagaaagccatccag |
| | tttactttgcagggcttcccaaccttaccagagggcgccccagctggcaattccggttcgcttgct |
| | gtccataaaaccgcccagtctagctatcgccatgtaagcccactgcaagctacctgctttctcttt |
| | gcgcttgcgttttcccttgtccagatagcccagtagctgacattcatccggggtcagcaccgtttc |
| | tgcggactggctttctacgtgttccgcttcctttagcagcccttgcgccatgcatttgttaaatcga |
| | attatccattgctgttgacaaagggaatcaggggatcttgaagttcctattccgaagttcctattctc |
| | tagaaagtataggaacttcagagcgcttttgaagctaattcgagatcatgcatgagctcactagtg |
| | gatccccccgacatcccaatacgcgtcaattcactggccgtcgttttactactttccgctgcataa |
| | ccctgcttcggggtcattatagcgattttttcggtatatccatcctttttcgcacgatatacaggattt |
| | tgccaaagggttcgtgtagactttccttggtgtatccaacggcgtcagccgggcaggataggtga |
| | agtaggcccacccgcgagcgggtgttccttcttcactgtcccttattcgcacctggcggtgctca |
| | acgggaatcctgctctgcgaggctggccgataagctctgataacgcaggaaagaacatgggc |
| | agttcaacctgttgatagtacgtactaagctctcatgtttcacgtactaagctctcaagtttaacgta |
| | ctaagctctcatgtttaacgaactaaaccctcatggctaacgtactaagctctcatggctaacgta |
| | ctaagctctcatgtttcacgtactaagctctcatgtttgaacaataaaattaatataaatcagcaactt |
| | aaatagcctctaaggttttaagttttataagaaaaaaaagaatatataaggcttttaaagcttttaag |
| | gtttaacggttgtggacaacaagccagggatgtaacgcactgagaagcccttagagcctctcaa |
| | agcaattttgagtgacacaggaacacttaacggctgacaaccggctggtaacaggattagcag |
| | agcgaggtatgtaggcggtgctacaagattgtactgagagtgcaccatacaggaaacagc |
| | (SEQ ID NO: 135) |
| pR6K- | gagagtgcaccatacaggaaacagctatgaccatgattacgccaagctcggcgcgccattgg |
| Donor(ZooTsy)_ | gattcggtcgaattcagagcgcttttgaagctgatgtgcttaaaaacttactcaatggaataattct |
| H1:12_ | agataattcactagtgattggaaatgttggatcctcatactcttcctttttcaattcagaagaactcgt |
| E1:0_E2:0_H2:12 | caagaaggcgatagaaggcgatgcgctgcgaatcgggagcggcgataccgtaaagcacga |
| | ggaagcggtcagcccattcgccgccaagctcttcagcaatatcacgggtagccaacgctatgt |
| | cctgatagcggtccgccacacccagccggccacagtcgatgaatccagaaaagcggccatttt |
| | ccaccatgatattcggcaagcaggcatcgccatgggtcacgacgagatcctcgccgtcgggc |
| | atgcgcgccttgagcctggcgaacagttcggctggcgcgagcccctgatgctcttcgtccaga |
| | tcatcctgatcgacaagaccggcttccatccgagtacgtgctcgctcgatgcgatgtttcgcttg |
| | gtggtcgaatgggcaggtagccggatcaagcgtatgcagccgccgcattgcatcagccatgat |
| | ggatactttctcggcaggagcaaggtgggatgacaggagatcctgccccggcacttcgccca |
| | atagcagccagtcccttcccgcttcagtgacaacgtcgagcacagctgcgcaaggaacgccc |
| | gtcgtggccagccacgatagccgcgctgcctcgtcctgcagttcattcagggcaccggacag |
| | gtcggtcttgacaaaaagaaccgggcgcccctgcgctgacagccggaacacggcggcatca |
| | gagcagccgattgtctgttgtgcccagtcatagccgaatagcctctccacccaagcggccgga |
| | gaacctgcgtgcaatccatcttgttcaatcatgcgaaacgatcctcatcctgtctcttgatcagatc |
| | ttgatcccctgcgccatcagatccttggcggcaagaaagccatccagtttactttgcagggcttc |
| | ccaaccttaccagagggcgccccagctggcaattccggttcgcttgctgtccataaaaccgccc |
| | agtctagctatcgccatgtaagcccactgcaagctacctgctttctctttgcgcttgcgttttccctt |
| | gtccagatagcccagtagctgacattcatccggggtcagcaccgtttctgcggactggctttcta |
| | cgtgttccgcttcctttagcagcccttgcgccatgcatttgttaaatcgaattatccattgctgttga |
| | caaagggaatcaggggatcttgaagttcctattccgaagttcctattctctagaaagtataggaac |
| | ttcagagcgcttttgaagctaattcgagatcatgcatgagctcactagtggatccccccgaccgg |
| | cagctcgtcatcccaatacgcgtcaattcactggccgtcgttttactactttccgctgcataaccct |
| | gcttcggggtcattatagcgattttttcggtatatccatcctttttcgcacgatatacaggattttgcc |
| | aaagggttcgtgtagactttccttggtgtatccaacggcgtcagccgggcaggataggtgaagt |
| | aggcccacccgcgagcgggtgttccttcttcactgtcccttattcgcacctggcggtgctcaacg |
| | ggaatcctgctctgcgaggctggccgataagctctgataacgcaggaaagaacatgggcagtt |
| | caacctgttgatagtacgtactaagctctcatgtttcacgtactaagctctcaagtttaacgtactaa |
| | gctctcatgtttaacgaactaaaccctcatggctaacgtactaagctctcatggctaacgtactaa |
| | gctctcatgtttcacgtactaagctctcatgtttgaacaataaaattaatataaatcagcaacttaaat |
| | agcctctaaggttttaagttttataagaaaaaaaagaatatataaggcttttaaagcttttaaggttta |
| | acggttgtggacaacaagccagggatgtaacgcactgagaagcccttagagcctctcaaagc |
| | aattttgagtgacacaggaacacttaacggctgacaaccggctggtaacaggattagcagagc |
| | gaggtatgtaggcggtgctacaagattgtactgagagtgcaccatacaggaaacagc (SEQ |
| | ID NO: 136) |
| pR6K- | gagagtgcaccatacaggaaacagctatgaccatgattacgccaagctcggcgcgccattgg |
| Donor(ZooTsy)_H1:0_ | gattcggtcgaattcagagcgcttttgaagctgatgtgcttaaaaacttactcaatggaataattct |
| E1:135_E2:0_H2:12 | agataattcactagtgattggaaatgttggatcctcatactcttcctttttcaattcagaagaactcgt |
| | caagaaggcgatagaaggcgatgcgctgcgaatcgggagcggcgataccgtaaagcacga |
| | ggaagcggtcagcccattcgccgccaagctcttcagcaatatcacgggtagccaacgctatgt |
| | cctgatagcggtccgccacacccagccggccacagtcgatgaatccagaaaagcggccatttt |
| | ccaccatgatattcggcaagcaggcatcgccatgggtcacgacgagatcctcgccgtcgggc |
| | atgcgcgccttgagcctggcgaacagttcggctggcgcgagcccctgatgctcttcgtccaga |
| | tcatcctgatcgacaagaccggcttccatccgagtacgtgctcgctcgatgcgatgtttcgcttg |
| | gtggtcgaatgggcaggtagccggatcaagcgtatgcagccgccgcattgcatcagccatgat |
| | ggatactttctcggcaggagcaaggtgggatgacaggagatcctgccccggcacttcgccca |
| | atagcagccagtcccttcccgcttcagtgacaacgtcgagcacagctgcgcaaggaacgccc |
| | gtcgtggccagccacgatagccgcgctgcctcgtcctgcagttcattcagggcaccggacag |
| | gtcggtcttgacaaaaagaaccgggcgcccctgcgctgacagccggaacacggcggcatca |
| | gagcagccgattgtctgttgtgcccagtcatagccgaatagcctctccacccaagcggccgga |
| | gaacctgcgtgcaatccatcttgttcaatcatgcgaaacgatcctcatcctgtctcttgatcagatc |
| | ttgatcccctgcgccatcagatccttggcggcaagaaagccatccagtttactttgcagggcttc |
| | ccaaccttaccagagggcgccccagctggcaattccggttcgcttgctgtccataaaaccgccc |
| | agtctagctatcgccatgtaagcccactgcaagctacctgctttctctttgcgcttgcgttttccctt |
| | gtccagatagcccagtagctgacattcatccggggtcagcaccgtttctgcggactggctttcta |
| | cgtgttccgcttcctttagcagcccttgcgccatgcatttgttaaatcgaattatccattgctgttga |
| | caaagggaatcaggggatcttgaagttcctattccgaagttcctattctctagaaagtataggaac |
| | ttcagagcgcttttgaagctaattcgagatcatgcatgagctcactagtggatccccccgactact |
| | tctaatgctgtgacatcactcacaggattcctgcaagtaatgcgacattggatactttgggattcct |
| | ttatagtcagtgggttatccgaatttttgccttgttcgttgctacattggctaaatcagaacttatccc |
| | aatacgcgtcaattcactggccgtcgttttactactttccgctgcataaccctgcttcggggtcatt |
| | atagcgattttttcggtatatccatcctttttcgcacgatatacaggattttgccaaagggttcgtgta |
| | gactttccttggtgtatccaacggcgtcagccgggcaggataggtgaagtaggcccacccgcg |
| | agcgggtgttccttcttcactgtcccttattcgcacctggcggtgctcaacgggaatcctgctctg |
| | cgaggctggccgataagctctgataacgcaggaaagaacatgggcagttcaacctgttgatag |
| | tacgtactaagctctcatgtttcacgtactaagctctcaagtttaacgtactaagctctcatgtttaac |
| | gaactaaaccctcatggctaacgtactaagctctcatggctaacgtactaagctctcatgtttcac |
| | gtactaagctctcatgtttgaacaataaaattaatataaatcagcaacttaaatagcctctaaggttt |
| | taagttttataagaaaaaaaagaatatataaggcttttaaagcttttaaggtttaacggttgtggaca |
| | acaagccagggatgtaacgcactgagaagcccttagagcctctcaaagcaattttgagtgaca |
| | caggaacacttaacggctgacaaccggctggtaacaggattagcagagcgaggtatgtaggc |
| | ggtgctacaagattgtactgagagtgcaccatacaggaaacagc (SEQ ID NO: 137) |
| pR6K- | gagagtgcaccatacaggaaacagctatgaccatgattacgccaagctcggcgcgccattgg |
| Donor(ZooTsy)_H1:0_ | gattcggtcgaattcagagcgcttttgaagctgatgtgcttaaaaacttactcaatggaataattct |
| E1:0_E2:0_H2:12 | agataattcactagtgattggaaatgttggatcctcatactcttcctttttcaattcagaagaactcgt |
| | caagaaggcgatagaaggcgatgcgctgcgaatcgggagcggcgataccgtaaagcacga |
| | ggaagcggtcagcccattcgccgccaagctcttcagcaatatcacgggtagccaacgctatgt |
| | cctgatagcggtccgccacacccagccggccacagtcgatgaatccagaaaagcggccatttt |
| | ccaccatgatattcggcaagcaggcatcgccatgggtcacgacgagatcctcgccgtcgggc |
| | atgcgcgccttgagcctggcgaacagttcggctggcgcgagcccctgatgctcttcgtccaga |
| | tcatcctgatcgacaagaccggcttccatccgagtacgtgctcgctcgatgcgatgtttcgcttg |
| | gtggtcgaatgggcaggtagccggatcaagcgtatgcagccgccgcattgcatcagccatgat |
| | ggatactttctcggcaggagcaaggtgggatgacaggagatcctgccccggcacttcgccca |
| | atagcagccagtcccttcccgcttcagtgacaacgtcgagcacagctgcgcaaggaacgccc |
| | gtcgtggccagccacgatagccgcgctgcctcgtcctgcagttcattcagggcaccggacag |
| | gtcggtcttgacaaaaagaaccgggcgcccctgcgctgacagccggaacacggcggcatca |
| | gagcagccgattgtctgttgtgcccagtcatagccgaatagcctctccacccaagcggccgga |
| | gaacctgcgtgcaatccatcttgttcaatcatgcgaaacgatcctcatcctgtctcttgatcagatc |
| | ttgatcccctgcgccatcagatccttggggcaagaaagccatccagtttactttgcagggcttc |
| | ccaaccttaccagagggcgccccagctggcaattccggttcgcttgctgtccataaaaccgccc |
| | agtctagctatcgccatgtaagcccactgcaagctacctgctttctctttgcgcttgcgttttccctt |
| | gtccagatagcccagtagctgacattcatccggggtcagcaccgtttctgcggactggctttcta |
| | cgtgttccgcttcctttagcagcccttgcgccatgcatttgttaaatcgaattatccattgctgttga |
| | caaagggaatcaggggatcttgaagttcctattccgaagttcctattctctagaaagtataggaac |
| | ttcagagcgcttttgaagctaattcgagatcatgcatgagctcactagtggatccccccgacatc |
| | ccaatacgcgtcaattcactggccgtcgttttactactttccgctgcataaccctgcttcggggtca |
| | ttatagcgattttttcggtatatccatcctttttcgcacgatatacaggattttgccaaagggttcgtg |
| | tagactttccttggtgtatccaacggcgtcagccgggcaggataggtgaagtaggcccacccg |
| | cgagcgggtgttccttcttcactgtcccttattcgcacctggcggtgctcaacgggaatcctgctc |
| | tgcgaggctggccgataagctctgataacgcaggaaagaacatgggcagttcaacctgttgat |
| | agtacgtactaagctctcatgtttcacgtactaagctctcaagtttaacgtactaagctctcatgttta |
| | acgaactaaaccctcatggctaacgtactaagctctcatggctaacgtactaagctctcatgtttc |
| | acgtactaagctctcatgtttgaacaataaaattaatataaatcagcaacttaaatagcctctaagg |
| | ttttaagttttataagaaaaaaaagaatatataaggcttttaaagcttttaaggtttaacggttgtgga |
| | caacaagccagggatgtaacgcactgagaagcccttagagcctctcaaagcaattttgagtga |
| | cacaggaacacttaacggctgacaaccggctggtaacaggattagcagagcgaggtatgtag |
| | gcggtgctacaagattgtactgagagtgcaccatacaggaaacagc (SEQ ID NO: |
| | 138) |
| pR6K- | gagagtgcaccatacaggaaacagctatgaccatgattacgccaagctcggcgcgccattgg |
| Donor(ZooTsy)_ | gatggactgggattctccaatatagagcgcttttgaagctgatgtgcttaaaaacttactcaatgg |
| H1:12_ | aataattctagataattcactagtgattggaaatgttggatcctcatactcttcctttttcaattcaga |
| E1:135_E2:20_H2:0 | agaactcgtcaagaaggcgatagaaggcgatgcgctgcgaatcgggagcggcgataccgtaa |
| | agcacgaggaagcggtcagcccattcgccgccaagctcttcagcaatatcacgggtagccaa |
| | cgctatgtcctgatagcggtccgccacacccagccggccacagtcgatgaatccagaaaagc |
| | ggccattttccaccatgatattcggcaagcaggcatcgccatgggtcacgacgagatcctcgcc |
| | gtcgggcatgcgcgccttgagcctggcgaacagttcggctggcgcgagcccctgatgctcttc |
| | gtccagatcatcctgatcgacaagaccggcttccatccgagtacgtgctcgctcgatgcgatgtt |
| | tcgcttggtggtcgaatgggcaggtagccggatcaagcgtatgcagccgccgcattgcatcag |
| | ccatgatggatactttctcggcaggagcaaggtgggatgacaggagatcctgccccggcactt |
| | cgcccaatagcagccagtcccttcccgcttcagtgacaacgtcgagcacagctgcgcaagga |
| | acgcccgtcgtggccagccacgatagccgcgctgcctcgtcctgcagttcattcagggcaccg |
| | gacaggtcggtcttgacaaaaagaaccgggcgcccctgcgctgacagccggaacacggcg |
| | gcatcagagcagccgattgtctgttgtgcccagtcatagccgaatagcctctccacccaagcgg |
| | ccggagaacctgcgtgcaatccatcttgttcaatcatgcgaaacgatcctcatcctgtctcttgat |
| | cagatcttgatcccctgcgccatcagatccttggcggcaagaaagccatccagtttactttgcag |
| | ggcttcccaaccttaccagagggcgccccagctggcaattccggttcgcttgctgtccataaaa |
| | ccgcccagtctagctatcgccatgtaagcccactgcaagctacctgctttctctttgcgcttgcgtt |
| | ttcccttgtccagatagcccagtagctgacattcatccggggtcagcaccgtttctgcggactgg |
| | ctttctacgtgttccgcttcctttagcagcccttgcgccatgcatttgttaaatcgaattatccattgc |
| | tgttgacaaagggaatcaggggatcttgaagttcctattccgaagttcctattctctagaaagtata |
| | ggaacttcagagcgcttttgaagctaattcgagatcatgcatgagctcactagtggatccccccg |
| | actacttctaatgctgtgacatcactcacaggattcctgcaagtaatgcgacattggatactttggg |
| | attcctttatagtcagtgggttatccgaatttttgccttgttcgttgctacattggctaaatcagaact |
| | tcggcagctcgtcatcccaatacgcgtcaattcactggccgtcgttttactactttccgctgcataa |
| | ccctgcttcggggtcattatagcgattttttcggtatatccatcctttttcgcacgatatacaggattt |
| | tgccaaagggttcgtgtagactttccttggtgtatccaacggcgtcagccgggcaggataggtga |
| | agtaggcccacccgcgagcgggtgttccttcttcactgtcccttattcgcacctggcggtgctca |
| | acgggaatcctgctctgcgaggctggccgataagctctgataacgcaggaaagaacatgggc |
| | agttcaacctgttgatagtacgtactaagctctcatgtttcacgtactaagctctcaagtttaacgta |
| | ctaagctctcatgtttaacgaactaaaccctcatggctaacgtactaagctctcatggctaacgta |
| | ctaagctctcatgtttcacgtactaagctctcatgtttgaacaataaaattaatataaatcagcaactt |
| | aaatagcctctaaggttttaagttttataagaaaaaaaagaatatataaggcttttaaagcttttaag |
| | gtttaacggttgtggacaacaagccagggatgtaacgcactgagaagcccttagagcctctcaa |
| | agcaattttgagtgacacaggaacacttaacggctgacaaccggctggtaacaggattagcag |
| | agcgaggtatgtaggcggtgctacaagattgtactgagagtgcaccatacaggaaacagc |
| | (SEQ ID NO: 139) |
| pR6K- | gagagtgcaccatacaggaaacagctatgaccatgattacgccaagctcggcgcgccattgg |
| Donor(ZooTsy)_ | gatggactgggattctccaatatagagcgcttttgaagctgatgtgcttaaaaacttactcaatgg |
| H1:12_ | aataattctagataattcactagtgattggaaatgttggatcctcatactcttcctttttcaattcaga |
| E1:0_E2:20_H2:0 | agaactcgtcaagaaggcgatagaaggcgatgcgctgcgaatcgggagcggcgataccgtaa |
| | agcacgaggaagcggtcagcccattcgccgccaagctcttcagcaatatcacgggtagccaa |
| | cgctatgtcctgatagcggtccgccacacccagccggccacagtcgatgaatccagaaaagc |
| | ggccattttccaccatgatattcggcaagcaggcatcgccatgggtcacgacgagatcctcgcc |
| | gtcgggcatgcgcgccttgagcctggcgaacagttcggctggcgcgagcccctgatgctcttc |
| | gtccagatcatcctgatcgacaagaccggcttccatccgagtacgtgctcgctcgatgcgatgtt |
| | tcgcttggtggtcgaatgggcaggtagccggatcaagcgtatgcagccgccgcattgcatcag |
| | ccatgatggatactttctcggcaggagcaaggtgggatgacaggagatcctgccccggcactt |
| | cgcccaatagcagccagtcccttcccgcttcagtgacaacgtcgagcacagctgcgcaagga |
| | acgcccgtcgtggccagccacgatagccgcgctgcctcgtcctgcagttcattcagggcaccg |
| | gacaggtcggtcttgacaaaaagaaccgggcgcccctgcgctgacagccggaacacggcg |
| | gcatcagagcagccgattgtctgttgtgcccagtcatagccgaatagcctctccacccaagcgg |
| | ccggagaacctgcgtgcaatccatcttgttcaatcatgcgaaacgatcctcatcctgtctcttgat |
| | cagatcttgatcccctgcgccatcagatccttggcggcaagaaagccatccagtttactttgcag |
| | ggcttcccaaccttaccagagggcgccccagctggcaattccggttcgcttgctgtccataaaa |
| | ccgcccagtctagctatcgccatgtaagcccactgcaagctacctgctttctctttgcgcttgcgtt |
| | ttcccttgtccagatagcccagtagctgacattcatccggggtcagcaccgtttctgcggactgg |
| | ctttctacgtgttccgcttcctttagcagcccttgcgccatgcatttgttaaatcgaattatccattgc |
| | tgttgacaaagggaatcaggggatcttgaagttcctattccgaagttcctattctctagaaagtata |
| | ggaacttcagagcgcttttgaagctaattcgagatcatgcatgagctcactagtggatccccccg |
| | accggcagctcgtcatcccaatacgcgtcaattcactggccgtcgttttactactttccgctgcat |
| | aaccctgcttcggggtcattatagcgattttttcggtatatccatcctttttcgcacgatatacagga |
| | ttttgccaaagggttcgtgtagactttccttggtgtatccaacggcgtcagccgggcaggatagg |
| | tgaagtaggcccacccgcgagcgggtgttccttcttcactgtcccttattcgcacctggcggtgc |
| | tcaacgggaatcctgctctgcgaggctggccgataagctctgataacgcaggaaagaacatgg |
| | gcagttcaacctgttgatagtacgtactaagctctcatgtttcacgtactaagctctcaagtttaacg |
| | tactaagctctcatgtttaacgaactaaaccctcatggctaacgtactaagctctcatggctaacgt |
| | actaagctctcatgtttcacgtactaagctctcatgtttgaacaataaaattaatataaatcagcaac |
| | ttaaatagcctctaaggttttaagttttataagaaaaaaaagaatatataaggcttttaaagcttttaa |
| | ggtttaacggttgtggacaacaagccagggatgtaacgcactgagaagcccttagagcctctc |
| | aaagcaattttgagtgacacaggaacacttaacggctgacaaccggctggtaacaggattagc |
| | agagcgaggtatgtaggcggtgctacaagattgtactgagagtgcaccatacaggaaacagc |
| | (SEQ ID NO: 140) |
| pR6K- | gagagtgcaccatacaggaaacagctatgaccatgattacgccaagctcggcgcgccattgg |
| Donor(ZooTsy)_H1:0_ | gatggactgggattctccaatatagagcgcttttgaagctgatgtgcttaaaaacttactcaatgg |
| E1:135_E2:20_H2:0 | aataattctagataattcactagtgattggaaatgttggatcctcatactcttcctttttcaattcaga |
| | agaactcgtcaagaaggcgatagaaggcgatgcgctgcgaatcgggagcggcgataccgtaa |
| | agcacgaggaagcggtcagcccattcgccgccaagctcttcagcaatatcacgggtagccaa |
| | cgctatgtcctgatagcggtccgccacacccagccggccacagtcgatgaatccagaaaagc |
| | ggccattttccaccatgatattcggcaagcaggcatcgccatgggtcacgacgagatcctcgcc |
| | gtcgggcatgcgcgccttgagcctggcgaacagttcggctggcgcgagcccctgatgctcttc |
| | gtccagatcatcctgatcgacaagaccggcttccatccgagtacgtgctcgctcgatgcgatgtt |
| | tcgcttggtggtcgaatgggcaggtagccggatcaagcgtatgcagccgccgcattgcatcag |
| | ccatgatggatactttctcggcaggagcaaggtgggatgacaggagatcctgccccggcactt |
| | cgcccaatagcagccagtcccttcccgcttcagtgacaacgtcgagcacagctgcgcaagga |
| | acgcccgtcgtggccagccacgatagccgcgctgcctcgtcctgcagttcattcagggcaccg |
| | gacaggtcggtcttgacaaaaagaaccgggcgcccctgcgctgacagccggaacacggcg |
| | gcatcagagcagccgattgtctgttgtgcccagtcatagccgaatagcctctccacccaagcgg |
| | ccggagaacctgcgtgcaatccatcttgttcaatcatgcgaaacgatcctcatcctgtctcttgat |
| | cagatcttgatcccctgcgccatcagatccttggcggcaagaaagccatccagtttactttgcag |
| | ggcttcccaaccttaccagagggcgccccagctggcaattccggttcgcttgctgtccataaaa |
| | ccgcccagtctagctatcgccatgtaagcccactgcaagctacctgctttctctttgcgcttgcgtt |
| | ttcccttgtccagatagcccagtagctgacattcatccggggtcagcaccgtttctgcggactgg |
| | ctttctacgtgttccgcttcctttagcagcccttgcgccatgcatttgttaaatcgaattatccattgc |
| | tgttgacaaagggaatcaggggatcttgaagttcctattccgaagttcctattctctagaaagtata |
| | ggaacttcagagcgcttttgaagctaattcgagatcatgcatgagctcactagtggatccccccg |
| | actacttctaatgctgtgacatcactcacaggattcctgcaagtaatgcgacattggatactttggg |
| | attcctttatagtcagtgggttatccgaatttttgccttgttcgttgctacattggctaaatcagaact |
| | tatcccaatacgcgtcaattcactggccgtcgttttactactttccgctgcataaccctgcttcgggg |
| | tcattatagcgattttttcggtatatccatcctttttcgcacgatatacaggattttgccaaagggttc |
| | gtgtagactttccttggtgtatccaacggcgtcagccgggcaggataggtgaagtaggcccac |
| | ccgcgagcgggtgttccttcttcactgtcccttattcgcacctggcggtgctcaacgggaatcct |
| | gctctgcgaggctggccgataagctctgataacgcaggaaagaacatgggcagttcaacctgt |
| | tgatagtacgtactaagctctcatgtttcacgtactaagctctcaagtttaacgtactaagctctcat |
| | gtttaacgaactaaaccctcatggctaacgtactaagctctcatggctaacgtactaagctctcat |
| | gtttcacgtactaagctctcatgtttgaacaataaaattaatataaatcagcaacttaaatagcctct |
| | aaggttttaagttttataagaaaaaaaagaatatataaggcttttaaagcttttaaggtttaacggtt |
| | gtggacaacaagccagggatgtaacgcactgagaagcccttagagcctctcaaagcaattttg |
| | agtgacacaggaacacttaacggctgacaaccggctggtaacaggattagcagagcgaggta |
| | tgtaggcggtgctacaagattgtactgagagtgcaccatacaggaaacagc (SEQ ID |
| | NO: 141) |
| pR6K- | Gagagtgcaccatacaggaaacagctatgaccatgattacgccaagctcggcgcgccattgg |
| Donor(ZooTsy)_H1:0_ | gatggactgggattctccaatatagagcgcttttgaagctgatgtgcttaaaaacttactcaatgg |
| E1:0_E2:20_H2:0 | aataattctagataattcactagtgattggaaatgttggatcctcatactcttcctttttcaattcaga |
| | agaactcgtcaagaaggcgatagaaggcgatgcgctgcgaatcgggagcggcgataccgtaa |
| | agcacgaggaagcggtcagcccattcgccgccaagctcttcagcaatatcacgggtagccaa |
| | cgctatgtcctgatagcggtccgccacacccagccggccacagtcgatgaatccagaaaagc |
| | ggccattttccaccatgatattcggcaagcaggcatcgccatgggtcacgacgagatcctcgcc |
| | gtcgggcatgcgcgccttgagcctggcgaacagttcggctggcgcgagcccctgatgctcttc |
| | gtccagatcatcctgatcgacaagaccggcttccatccgagtacgtgctcgctcgatgcgatgtt |
| | tcgcttggtggtcgaatgggcaggtagccggatcaagcgtatgcagccgccgcattgcatcag |
| | ccatgatggatactttctcggcaggagcaaggtgggatgacaggagatcctgccccggcactt |
| | cgcccaatagcagccagtcccttcccgcttcagtgacaacgtcgagcacagctgcgcaagga |
| | acgcccgtcgtggccagccacgatagccgcgctgcctcgtcctgcagttcattcagggcaccg |
| | gacaggtcggtcttgacaaaaagaaccgggcgcccctgcgctgacagccggaacacggcg |
| | gcatcagagcagccgattgtctgttgtgcccagtcatagccgaatagcctctccacccaagcgg |
| | ccggagaacctgcgtgcaatccatcttgttcaatcatgcgaaacgatcctcatcctgtctcttgat |
| | cagatcttgatcccctgcgccatcagatccttggcggcaagaaagccatccagtttactttgcag |
| | ggcttcccaaccttaccagagggcgccccagctggcaattccggttcgcttgctgtccataaaa |
| | ccgcccagtctagctatcgccatgtaagcccactgcaagctacctgctttctctttgcgcttgcgtt |
| | ttcccttgtccagatagcccagtagctgacattcatccggggtcagcaccgtttctgcggactgg |
| | ctttctacgtgttccgcttcctttagcagcccttgcgccatgcatttgttaaatcgaattatccattgc |
| | tgttgacaaagggaatcaggggatcttgaagttcctattccgaagttcctattctctagaaagtata |
| | ggaacttcagagcgcttttgaagctaattcgagatcatgcatgagctcactagtggatccccccg |
| | acatcccaatacgcgtcaattcactggccgtcgttttactactttccgctgcataaccctgcttcgg |
| | ggtcattatagcgattttttcggtatatccatcctttttcgcacgatatacaggattttgccaaagggt |
| | tcgtgtagactttccttggtgtatccaacggcgtcagccgggcaggataggtgaagtaggccca |
| | cccgcgagcgggtgttccttcttcactgtcccttattcgcacctggcggtgctcaacgggaatcc |
| | tgctctgcgaggctggccgataagctctgataacgcaggaaagaacatgggcagttcaacctg |
| | ttgatagtacgtactaagctctcatgtttcacgtactaagctctcaagtttaacgtactaagctctcat |
| | gtttaacgaactaaaccctcatggctaacgtactaagctctcatggctaacgtactaagctctcat |
| | gtttcacgtactaagctctcatgtttgaacaataaaattaatataaatcagcaacttaaatagcctct |
| | aaggttttaagttttataagaaaaaaaagaatatataaggcttttaaagcttttaaggtttaacggtt |
| | gtggacaacaagccagggatgtaacgcactgagaagcccttagagcctctcaaagcaattttg |
| | agtgacacaggaacacttaacggctgacaaccggctggtaacaggattagcagagcgaggta |
| | tgtaggcggtgctacaagattgtactgagagtgcaccatacaggaaacagc (SEQ ID |
| | NO: 142) |
| pR6K- | gagagtgcaccatacaggaaacagctatgaccatgattacgccaagctcggcgcgccattgg |
| Donor(ZooTsy)_ | gatagagcgcttttgaagctgatgtgcttaaaaacttactcaatggaataattctagataattcacta |
| H1:12_ | gtgattggaaatgttggatcctcatactcttcctttttcaattcagaagaactcgtcaagaaggcga |
| E1:135_E2:0_H2:0 | tagaaggcgatgcgctgcgaatcgggagcggcgataccgtaaagcacgaggaagcggtca |
| | gcccattcgccgccaagctcttcagcaatatcacgggtagccaacgctatgtcctgatagcggt |
| | ccgccacacccagccggccacagtcgatgaatccagaaaagcggccattttccaccatgatat |
| | tcggcaagcaggcatcgccatgggtcacgacgagatcctcgccgtcgggcatgcgcgccttg |
| | agcctggcgaacagttcggctggcgcgagcccctgatgctcttcgtccagatcatcctgatcga |
| | caagaccggcttccatccgagtacgtgctcgctcgatgcgatgtttcgcttggtggtcgaatgg |
| | gcaggtagccggatcaagcgtatgcagccgccgcattgcatcagccatgatggatactttctcg |
| | gcaggagcaaggtgggatgacaggagatcctgccccggcacttcgcccaatagcagccagt |
| | cccttcccgcttcagtgacaacgtcgagcacagctgcgcaaggaacgcccgtcgtggccagc |
| | cacgatagccgcgctgcctcgtcctgcagttcattcagggcaccggacaggtcggtcttgaca |
| | aaaagaaccgggcgcccctgcgctgacagccggaacacggcggcatcagagcagccgatt |
| | gtctgttgtgcccagtcatagccgaatagcctctccacccaagcggccggagaacctgcgtgc |
| | aatccatcttgttcaatcatgcgaaacgatcctcatcctgtctcttgatcagatcttgatcccctgcg |
| | ccatcagatccttggcggcaagaaagccatccagtttactttgcagggcttcccaaccttaccag |
| | agggcgccccagctggcaattccggttcgcttgctgtccataaaaccgcccagtctagctatcg |
| | ccatgtaagcccactgcaagctacctgctttctctttgcgcttgcgttttcccttgtccagatagccc |
| | agtagctgacattcatccggggtcagcaccgtttctgcggactggctttctacgtgttccgcttcc |
| | tttagcagcccttgcgccatgcatttgttaaatcgaattatccattgctgttgacaaagggaatcag |
| | gggatcttgaagttcctattccgaagttcctattctctagaaagtataggaacttcagagcgctttt |
| | gaagctaattcgagatcatgcatgagctcactagtggatccccccgactacttctaatgctgtga |
| | catcactcacaggattcctgcaagtaatgcgacattggatactttgggattcctttatagtcagtgg |
| | gttatccgaatttttgccttgttcgttgctacattggctaaatcagaacttcggcagctcgtcatccc |
| | aatacgcgtcaattcactggccgtcgttttactactttccgctgcataaccctgcttcggggtcatt |
| | atagcgattttttcggtatatccatcctttttcgcacgatatacaggattttgccaaagggttcgtgta |
| | gactttccttggtgtatccaacggcgtcagccgggcaggataggtgaagtaggcccacccgcg |
| | agcgggtgttccttcttcactgtcccttattcgcacctggcggtgctcaacgggaatcctgctctg |
| | cgaggctggccgataagctctgataacgcaggaaagaacatgggcagttcaacctgttgatag |
| | tacgtactaagctctcatgtttcacgtactaagctctcaagtttaacgtactaagctctcatgtttaac |
| | gaactaaaccctcatggctaacgtactaagctctcatggctaacgtactaagctctcatgtttcac |
| | gtactaagctctcatgtttgaacaataaaattaatataaatcagcaacttaaatagcctctaaggttt |
| | taagttttataagaaaaaaaagaatatataaggcttttaaagcttttaaggtttaacggttgtggaca |
| | acaagccagggatgtaacgcactgagaagcccttagagcctctcaaagcaattttgagtgaca |
| | caggaacacttaacggctgacaaccggctggtaacaggattagcagagcgaggtatgtaggc |
| | ggtgctacaagattgtactgagagtgcaccatacaggaaacagc (SEQ ID NO: 143) |
| pR6K- | gagagtgcaccatacaggaaacagctatgaccatgattacgccaagctcggcgcgccattgg |
| Donor(ZooTsy)_ | gatagagcgcttttgaagctgatgtgcttaaaaacttactcaatggaataattctagataattcacta |
| H1:12_ | gtgattggaaatgttggatcctcatactcttcctttttcaattcagaagaactcgtcaagaaggcga |
| E1:0_E2:0_H2:0 | tagaaggcgatgcgctgcgaatcgggagcggcgataccgtaaagcacgaggaagcggtca |
| | gcccattcgccgccaagctcttcagcaatatcacgggtagccaacgctatgtcctgatagcggt |
| | ccgccacacccagccggccacagtcgatgaatccagaaaagcggccattttccaccatgatat |
| | tcggcaagcaggcatcgccatgggtcacgacgagatcctcgccgtcgggcatgcgcgccttg |
| | agcctggcgaacagttcggctggcgcgagcccctgatgctcttcgtccagatcatcctgatcga |
| | caagaccggcttccatccgagtacgtgctcgctcgatgcgatgtttcgcttggtggtcgaatgg |
| | gcaggtagccggatcaagcgtatgcagccgccgcattgcatcagccatgatggatactttctcg |
| | gcaggagcaaggtgggatgacaggagatcctgccccggcacttcgcccaatagcagccagt |
| | cccttcccgcttcagtgacaacgtcgagcacagctgcgcaaggaacgcccgtcgtggccagc |
| | cacgatagccgcgctgcctcgtcctgcagttcattcagggcaccggacaggtcggtcttgaca |
| | aaaagaaccgggcgcccctgcgctgacagccggaacacggcggcatcagagcagccgatt |
| | gtctgttgtgcccagtcatagccgaatagcctctccacccaagcggccggagaacctgcgtgc |
| | aatccatcttgttcaatcatgcgaaacgatcctcatcctgtctcttgatcagatcttgatcccctgcg |
| | ccatcagatccttggcggcaagaaagccatccagtttactttgcagggcttcccaaccttaccag |
| | agggcgccccagctggcaattccggttcgcttgctgtccataaaaccgcccagtctagctatcg |
| | ccatgtaagcccactgcaagctacctgctttctctttgcgcttgcgttttcccttgtccagatagccc |
| | agtagctgacattcatccggggtcagcaccgtttctgcggactggctttctacgtgttccgcttcc |
| | tttagcagcccttgcgccatgcatttgttaaatcgaattatccattgctgttgacaaagggaatcag |
| | gggatcttgaagttcctattccgaagttcctattctctagaaagtataggaacttcagagcgctttt |
| | gaagctaattcgagatcatgcatgagctcactagtggatccccccgaccggcagctcgtcatcc |
| | caatacgcgtcaattcactggccgtcgttttactactttccgctgcataaccctgcttcggggtcat |
| | tatagcgattttttcggtatatccatcctttttcgcacgatatacaggattttgccaaagggttcgtgt |
| | agactttccttggtgtatccaacggcgtcagccgggcaggataggtgaagtaggcccacccgc |
| | gagcgggtgttccttcttcactgtcccttattcgcacctggcggtgctcaacgggaatcctgctct |
| | gcgaggctggccgataagctctgataacgcaggaaagaacatgggcagttcaacctgttgata |
| | gtacgtactaagctctcatgtttcacgtactaagctctcaagtttaacgtactaagctctcatgtttaa |
| | cgaactaaaccctcatggctaacgtactaagctctcatggctaacgtactaagctctcatgtttca |
| | cgtactaagctctcatgtttgaacaataaaattaatataaatcagcaacttaaatagcctctaaggtt |
| | ttaagttttataagaaaaaaaagaatatataaggcttttaaagcttttaaggtttaacggttgtggac |
| | aacaagccagggatgtaacgcactgagaagcccttagagcctctcaaagcaattttgagtgac |
| | acaggaacacttaacggctgacaaccggctggtaacaggattagcagagcgaggtatgtagg |
| | cggtgctacaagattgtactgagagtgcaccatacaggaaacagc (SEQ ID NO: 144) |
| pR6K- | gagagtgcaccatacaggaaacagctatgaccatgattacgccaagctcggcgcgccattgg |
| Donor(ZooTsy)_H1:0_ | gatagagcgcttttgaagctgatgtgcttaaaaacttactcaatggaataattctagataattcacta |
| E1:135_E2:0_H2:0 | gtgattggaaatgttggatcctcatactcttcctttttcaattcagaagaactcgtcaagaaggcga |
| | tagaaggcgatgcgctgcgaatcgggagcggcgataccgtaaagcacgaggaagcggtca |
| | gcccattcgccgccaagctcttcagcaatatcacgggtagccaacgctatgtcctgatagcggt |
| | ccgccacacccagccggccacagtcgatgaatccagaaaagcggccattttccaccatgatat |
| | tcggcaagcaggcatcgccatgggtcacgacgagatcctcgccgtcgggcatgcgcgccttg |
| | agcctggcgaacagttcggctggcgcgagcccctgatgctcttcgtccagatcatcctgatcga |
| | caagaccggcttccatccgagtacgtgctcgctcgatgcgatgtttcgcttggtggtcgaatgg |
| | gcaggtagccggatcaagcgtatgcagccgccgcattgcatcagccatgatggatactttctcg |
| | gcaggagcaaggtgggatgacaggagatcctgccccggcacttcgcccaatagcagccagt |
| | cccttcccgcttcagtgacaacgtcgagcacagctgcgcaaggaacgcccgtcgtggccagc |
| | cacgatagccgcgctgcctcgtcctgcagttcattcagggcaccggacaggtcggtcttgaca |
| | aaaagaaccgggcgcccctgcgctgacagccggaacacggcggcatcagagcagccgatt |
| | gtctgttgtgcccagtcatagccgaatagcctctccacccaagcggccggagaacctgcgtgc |
| | aatccatcttgttcaatcatgcgaaacgatcctcatcctgtctcttgatcagatcttgatcccctgcg |
| | ccatcagatccttggcggcaagaaagccatccagtttactttgcagggcttcccaaccttaccag |
| | agggcgccccagctggcaattccggttcgcttgctgtccataaaaccgcccagtctagctatcg |
| | ccatgtaagcccactgcaagctacctgctttctctttgcgcttgcgttttcccttgtccagatagccc |
| | agtagctgacattcatccggggtcagcaccgtttctgcggactggctttctacgtgttccgcttcc |
| | tttagcagcccttgcgccatgcatttgttaaatcgaattatccattgctgttgacaaagggaatcag |
| | gggatcttgaagttcctattccgaagttcctattctctagaaagtataggaacttcagagcgctttt |
| | gaagctaattcgagatcatgcatgagctcactagtggatccccccgactacttctaatgctgtga |
| | catcactcacaggattcctgcaagtaatgcgacattggatactttgggattcctttatagtcagtgg |
| | gttatccgaatttttgccttgttcgttgctacattggctaaatcagaacttatcccaatacgcgtcaat |
| | tcactggccgtcgttttactactttccgctgcataaccctgcttcggggtcattatagcgattttttcg |
| | gtatatccatcctttttcgcacgatatacaggattttgccaaagggttcgtgtagactttccttggtgt |
| | atccaacggcgtcagccgggcaggataggtgaagtaggcccacccgcgagcgggtgttcctt |
| | cttcactgtcccttattcgcacctggcggtgctcaacgggaatcctgctctgcgaggctggccg |
| | ataagctctgataacgcaggaaagaacatgggcagttcaacctgttgatagtacgtactaagctc |
| | tcatgtttcacgtactaagctctcaagtttaacgtactaagctctcatgtttaacgaactaaaccctc |
| | atggctaacgtactaagctctcatggctaacgtactaagctctcatgtttcacgtactaagctctca |
| | tgtttgaacaataaaattaatataaatcagcaacttaaatagcctctaaggttttaagttttataagaa |
| | aaaaaagaatatataaggcttttaaagcttttaaggtttaacggttgtggacaacaagccagggat |
| | gtaacgcactgagaagcccttagagcctctcaaagcaattttgagtgacacaggaacacttaac |
| | ggctgacaaccggctggtaacaggattagcagagcgaggtatgtaggcggtgctacaagatt |
| | gtactgagagtgcaccatacaggaaacagc (SEQ ID NO: 145) |
| pTarget(ZooTsy) | Cacggcttcgaccgcagcactggtcggtggaggcggcacgccccgccccggcgagatcag |
| | cctggcgcacaacggcgtgctgtacctggacgagctgccggaattcgaccgacgtgtgctcg |
| | aggccctgcgcgagccgctggaaagcggccacatcacgatttcccgtgcagcccgccgcgc |
| | cgagttcccggcccgctctaggctgctgccaccgctgagcaataactagcataaccccttggg |
| | gcctctaaacgggtcttgaggggttttttgctgaaacctcaggcatttgagaagcacacggtcac |
| | actgcttccggtagtcaataaaccggtaaaccagcaatagacataagcggctatttaacgaccct |
| | gccctgaaccgacgaccgggtcgaatttgctttcgaatttctgccattcatccgcttattatcactta |
| | ttcaggcgtagcaccaggcgtttaagggcaccaataactgccttaaaaaaattacgccccgccc |
| | tgccactcatcgcagtactgttgtaattcattaagcattctgccgacatggaagccatcacagacg |
| | gcatgatgaacctgaatcgccagcggcatcagcaccttgtcgccttgcgtataatatttgcccat |
| | agtgaaaacgggggcgaagaagttgtccatattggccacgtttaaatcaaaactggtgaaactc |
| | acccagggattggctgagacgaaaaacatattctcaataaaccctttagggaaataggccaggt |
| | tttcaccgtaacacgccacatcttgcgaatatatgtgtagaaactgccggaaatcgtcgtggtatt |
| | cactccagagcgatgaaaacgtttcagtttgctcatggaaaacggtgtaacaagggtgaacact |
| | atcccatatcaccagctcaccgtctttcattgccatacggaactccggatgagcattcatcaggc |
| | gggcaagaatgtgaataaaggccggataaaacttgtgcttatttttctttacggtctttaaaaaggc |
| | cgtaatatccagctgaacggtctggttataggtacattgagcaactgactgaaatgcctcaaaat |
| | gttctttacgatgccattgggatatatcaacggtggtatatccagtgatttttttctccattttagcttc |
| | cttagctcctgaaaatctcgataactcaaaaaatacgcccggtagtgatcttatttcattatggtga |
| | aagttggaacctcttacgtgccgatcaacgtctcattttcgccaaaagttggcccagggcttccc |
| | ggtatcaacagggacaccaggatttatttattctgcgaagtgatcttccgtcacaggtatttattcg |
| | gcgcaaagtgcgtcgggtgatgctgccaacttactgatttagtgtatgatggtgtttttgaggtgct |
| | ccagtggcttctgtttctatcagctgtccctcctgttcagctactgacggggtggtgcgtaacggc |
| | aaaagcaccgccggacatcagcgctagcggagtgtatactggcttactatgttggcactgatga |
| | gggtgtcagtgaagtgcttcatgtggcaggagaaaaaaggctgcaccggtgcgtcagcagaa |
| | tatgtgatacaggatatattccgcttcctcgctcactgactcgctacgctcggtcgttcgactgcg |
| | gcgagcggaaatggcttacgaacggggcggagatttcctggaagatgccaggaagatactta |
| | acagggaagtgagagggccgcggcaaagccgtttttccataggctccgcccccctgacaagc |
| | atcacgaaatctgacgctcaaatcagtggtggcgaaacccgacaggactataaagataccagg |
| | cgtttcccctggcggctccctcgtgcgctctcctgttcctgcctttcggtttaccggtgtcattccg |
| | ctgttatggccgcgtttgtctcattccacgcctgacactcagttccgggtaggcagttcgctccaa |
| | gctggactgtatgcacgaaccccccgttcagtccgaccgctgcgccttatccggtaactatcgtc |
| | ttgagtccaacccggaaagacatgcaaaagcaccactggcagcagccactggtaattgattta |
| | gaggagttagtcttgaagtcatgcgccggttaaggctaaactgaaaggacaagttttggtgact |
| | gcgctcctccaagccagttacctcggttcaaagagttggtagctcagagaaccttcgaaaaacc |
| | gccctgcaaggcggttttttcgttttcagagcaagagattacgcgcagaccaaaacgatctcaa |
| | gaagatcatcttattaatcagataaaatatttctagatttcagtgcaatttatctcttcaaatgtagcac |
| | ctgaagtcagccccatacgatataagttgtaattctcatgttagtcatgccccgcgcccaccgga |
| | aggagctgactgggttgaaggctctcaagggcatcggtcgagatcccggtgcctaatgagtga |
| | gctaacttacattaattgcgttgcgc (SEQ ID NO: 146) |

E. coli Transposition Assays

For CyCAST, 100 ng of pHelper (CyCAST) was co-electroporated with 100 ng of pDonor (CyCAST) and 100 ng of pTarget (CyCAST) into BL21 (DE3) electrocompetent cells (Sigma) and plated on 100 μg/ml carbenicillin, 50 μg/ml spectinomycin, and 50 μg/ml chloramphenicol containing LB-agar plates. After incubation for 17 hours at 37° C., all colonies were scraped from the plates, and a portion was re-plated on 0.1 mM IPTG supplemented triple antibiotic LB-agar plates to induce protein expression. Cells were incubated for an additional 17 hours at 37° C. All colonies were scraped, and the plasmid DNA was purified using QIAprep Spin Miniprep Kit (QIAGEN). Insertions were identified by PCR using Q5 High-Fidelity DNA polymerase (NEB). Insertion products containing the 6N sequence were amplified and sequenced using a MiSeq Reagent Kit v2, 300-cycle (Illumina). PAM and insert position were characterized with the established pipeline (Strecker, J. et al. RNA-guided DNA insertion with CRISPR-associated transposases. Science 365, 48-53 (2019)). The frequency of insertions was determined with droplet digital PCR (ddPCR) as described below with 1 μg to 10 ng template plasmid DNA for 20 μL ddPCR reaction. AjTn6022 and ZooTsy were analyzed in the same manner, but a constitutive lac promoter was used for expression of proteins. In brief, co-electroporated transformants were incubated at triple antibiotics 50 μg/ml kanamycin, 50 μg/ml spectinomycin, and 50 μg/ml chloramphenicol containing LB-agar plates for 17 hours at 37° C., and all colonies were scraped for the downstream analysis. Experiments were performed with three biological replicates. All data points are shown with an error bar showing standard deviation, and statistical significance was assessed by t-test.

### Nanopore Long-Read Sequencing to Determine the Plasmid Structure

To isolate pInsert and determine its structure, the pR6K-donor was utilized. For AjTn6022, pHelper (AjTn6022), pR6K-donor (AjTn6022), and pTarget (AjTn6022) were co-electroporated into Pir1+E. coli. Cells were recovered for 1 hour and plated on 50 μg/ml kanamycin, 50 μg/ml spectinomycin, and 50 μg/ml chloramphenicol containing LB-agar plates. Cells were harvested 5 days after incubation at room temperature and subjected to mini-prep by QIAprep Spin Miniprep Kit (Qiagen). Incubation at 37° C. was avoided to prevent transposed products being actively resolved to enable detection of replicative transposition. To recover transposed pInsert products, 100 ng mini-prep product was electroporated into Endura Competent Cells (Lucigen). Cells were recovered for 1 hour and plated on 50 μg/ml kanamycin and 50 μg/ml chloramphenicol containing LB-agar plates, and further incubated at room temperature for 5 days. Donor insertion on pTarget was confirmed by Sanger sequencing of mini-prep products for 4 colonies. In parallel, all colonies were harvested and subjected to mini-prep, followed by amplification-free nanopore sequencing library preparation (Oxford Nanopore Technologies SQK-LSK109). Briefly, mini-prep products were linearized by BstZ17I, followed by end-prep and subsequent ligation of sequencing adapters. Resulting libraries were loaded on a MinION R9 flow cell and sequenced. Sequence reads containing 25 bp of comM gene fragment, 25 bp of LE and 25 bp of RE (up to 2 bp mismatches were allowed for each component) were filtered for further analysis, thereby discarding low-quality and contaminating gDNA reads. ZooTsy was analyzed in the same manner with cognate plasmids. For quality control, reads over 2.5 kb with QScore >7 were filtered. To determine the structure of pInsert, the reads were mapped on the expected simple insertion or excised cointegrate products (FIG. 12D). To count simple insertions and excised cointegrates, nanopore sequencing reads were analyzed using the following pipeline (FIGS. 12C and 12E): the reads which contain both (1) the downstream edge 30-bp sequence of cloned comM 100-bp fragment (AGCGGGCCGGGAACTCGGCGCGGCGGGCTG (SEQ ID NO: 147)) and (2)30-bp end2 sequence (GGACTGGGATTCTCCAATATTCCTTAGCGC (SEQ ID NO: 148)) were further extracted by the insertion reads counter. Among those reads, the ones which have (3) the upstream edge 30-bp sequence of the cloned comM fragment (CACGGCTTCGACCGCAGCACTGGTCGGTGG (SEQ ID NO: 149)) and (4) the 30-bp sequence of the cargo (GTCGGGGGGATCCACTAGTGAGCTCATGCA (SEQ ID NO: 150)) were extracted by the counter and used for mapping on the expected simple insertion or excised cointegrate products. Based on the distance between the upstream edge of comM and cargo, each selected read was counted as either a simple insertion or excised cointegrate.

### Tsy Circular Intermediate Isolation Assays

100 ng of pHelper (ZooTsy) was co-electroporated with 100 ng of lacZα backbone pDonor (ZooTsy) and 100 ng of pACYC backbone vector into BL21 (DE3) electrocompetent cells (Sigma) and plated on 50 μg/ml kanamycin, 50 μg/ml spectinomycin, and 50 μg/ml chloramphenicol containing LB-agar plates. After incubation for 17 hours at 37° C., all colonies were scraped from the plates, and the plasmid DNA was purified using QIAprep Spin Miniprep Kit (QIAGEN). 100 μg of mini-prep product was used for re-transformation of NEB 10-beta Competent E. coli (NEB), and the cells were plated on S-Gal (Sigma) and 50 μg/ml kanamycin containing LB-agar plates for blue/white selection. After incubation for 17 hours at 37° C., white colonies were picked up and further cultured for 17 hours at 37° C., and the plasmid DNA was purified using QIAprep Spin Miniprep Kit (QIAGEN). The original lacZα backbone pDonor (ZooTsy) and the circularized donor (cd) were digested with NruI (NEB) for an hour at 37° C. and loaded on E-Gel Ex Agarose Gels 1%. The junction of end1 and end2 was confirmed by Sanger sequencing of mini-prep products for 4 white colonies. In parallel, isolated products from 24 white colonies were combined and subjected to amplification-free nanopore sequencing library preparation (Oxford Nanopore Technologies SQK-LSK109). Briefly, mini-prep products were linearized by NruI, followed by end-prep and subsequent ligation of sequencing adapters. Resulting libraries were loaded on a MinION R9 flow cell and sequenced.

### TTISS for Insertion Specificity Analysis

Endura Competent Cells were transformed for transposition assay as mentioned above, except following; 100 ng of a temperature sensitive pSC101-donor was used. After incubation on triple antibiotic LB-agar plates, cells were re-plated and grown for 12 hours at 43° C. to prevent unintended amplification of donor plasmids in the following Tagmentation-based Tag Integration Site Sequencing (TTISS) analysis (Schmid-Burgk, J. L. et al. Highly Parallel Profiling of Cas9 Variant Specificity. Mol. Cell 78, 794-800.e8 (2020)). Genomic DNA was extracted from hundreds of colonies on a LB-agar petri dish, and 500 ng genomic DNA (from approximate 108 E. coli cells) was tagmented with Tn5, and re-purified by Wizard SV Gel and PCR Clean-Up System (Promega). Tagmented DNA samples were amplified using two rounds (in total 37 cycles) of PCR with KOD Hot Start DNA Polymerase (Millipore) using a Tn5 adapter-specific primer and nested primers within the DNA donor Table S2). PCR products from 4 different experimental conditions were pooled together, purified, and the resulting libraries were sequenced using a NextSeq v2 kit, 75 cycle kit with 45 forward cycles and 30 reverse cycles. Read pairs with RI containing the terminal 29 bp of the AjTn6022 transposon RE sequence were filtered for further analysis and trimmed of the transposon sequence for alignment to the E. coli genome (CP011113.2) and pTarget (AjTn6022). Filtered and trimmed reads were aligned using the established BWA aligner pipeline (Altae-Tran, H. et al. The widespread IS200/IS605 transposon family encodes diverse programmable RNA-guided endonucleases. Science 374, 57-65 (2021)). The resulting SAM files were exported for further analysis. Aligned RI reads with length of 16 bp (remaining RI length after trimming of 29 bp of transposon RE sequence) and SAM flags 99 and 147 (for mapped reads within the insert size and in correct orientation) were used to determine the correct transposon insertion reads. Reads with a single insertion position at the Aj comM on pTarget and E. coli endogenous comM insertion sites were considered on-target, while remaining reads were counted as off-targets. For ZooTsy, the resulting libraries were sequenced using a MiSeq v2 kit with 75 forward cycles and 75 reverse cycles. Read pairs with RI containing the terminal 36 bp of the ZooTsy endl sequence were filtered for further analysis and trimmed of the transposon sequence for alignment to the E. coli genome and pTarget (ZooTsy). Filtered and trimmed reads were aligned, and the resulting SAM files were exported for further analysis. Aligned R1 reads with length of 39 bp (remaining RI length after trimming of 36 bp of transposon endl sequence) and SAM flags 99 and 147 were used to determine the correct transposon insertion reads.

Droplet Digital POR (ddPCR) Reactions

Insertion events were quantified using insertion specific primers (Table 8) and a donor specific probe (Table 9). ddPCR Supermix for Probes (No dUTP) (BioRad), primers (900 nM each), a probe (250 nM), and template DNA were combined into 20 mL reactions, and droplets were generated with 70 μL of Droplet Generation Oil for Probes (BioRad) using the QX200 Droplet Generator (BioRad). Thermal cycling conditions for ddPCR reactions were as follows: 1 cycle, 95° C., 10 min; 40 cycles, 94° C., 30 s, 58° C., 1 min; 1 cycle, 98° C., 10 mins; 4° C. hold. PCR products were read with a QX200 Droplet Reader, and absolute concentrations of inserts were determined using QuantaSoft (v1.6.6.0320). Total template (genome or target plasmid) amount was also quantified through this process, and insertion frequency was calculated as inserts/template.

TABLE 8

| Primer | |
|---|---|
| description | Primer sequence |
| NGS pTarget | ctttccctacacgacgctcttccga |
| 6N-upstream | tctgagcaagagattacgcgcagac |
| | (SEQ ID NO: 151) |
| NGS pTarget | ctttccctacacgacgctcttccga |
| 6N-downstream | tctctaccgcattaaagcttccgcc |
| | (SEQ ID NO: 152) |
| NGS pDonor | gactggagttcagacgtgtgctctt |
| (CyCAST)-LE | ccgatctgcctattgctttcgctct |
| | atctgtcc |
| | (SEQ ID NO: 153) |
| NGS pDonor | gactggagttcagacgtgtgctctt |
| (CyCAST)-RE | ccgatctcctaaggcaacacaacgg |
| | ctg |
| | (SEQ ID NO: 154) |
| NGS tRNA-leu | ctttccctacacgacgctcttccga |
| | tctgtaagccttgcgagttcgattc |
| | tcg |
| | (SEQ ID NO: 155) |
| pTarget ddPCR | ctaccgcattaaagcttccgcc |
| 6N-downstream | (SEQ ID NO: 156) |
| pDonor(CyCAST) | cctaaggcaacacaacggctg |
| ddPCR for RE | (SEQ ID NO: 157) |
| pDonor(CyCAST) | cctggtgtccctgttgataccg |
| ddPCR | (SEQ ID NO: 158) |
| tRNA-leu | |
| downstream | |
| pTarget ddPCR | cgacagcatcgccagtcactatg |
| for TcR1 | (SEQ ID NO: 159) |
| pTarget ddPCR | caagtagcgaagcgagcaggac |
| for TcR2 | (SEQ ID NO: 160) |
| NGS AjTn6022- | ctttccctacacgacgctcttccga |
| comM upstream | tctagcttccttagctcctgaaaat |
| | ctcg |
| | (SEQ ID NO: 161) |
| NGS AjTn6022- | ctttccctacacgacgctcttccga |
| comM | tctgaaaatgagacgttgatcggca |
| downstream | c |
| | (SEQ ID NO: 162) |
| NGS pDonor | gactggagttcagacgtgtgctctt |
| (AjTn6022)-RE | ccgatctgccactcttgcttattac |
| | tgtc |
| | (SEQ ID NO: 163) |
| NGS pDonor | gactggagttcagacgtgtgctctt |
| (AjTn6022)-LE | ccgatctttgcaggcatatctttta |
| | gtg |
| | (SEQ ID NO: 164) |
| pDonor | gccactcttgcttattactgtc |
| (AjTn6022) | (SEQ ID NO: 165) |
| ddPCR forRE | |
| pTarget ddPCR | gaaaatgagacgttgatcggcac |
| Aj-comM | (SEQ ID NO: 166) |
| downstream | |
| NGS ZooTsy- | ctttccctacacgacgctcttccga |
| comM upstream | tctctgggttgaaggctctcaaggg |
| | c |
| | (SEQ ID NO: 167) |
| NGS ZooTsy- | ctttccctacacgacgctcttccga |
| comM | tctgtgtgcttctcaaatgcctgag |
| downstream | gtttc |
| | (SEQ ID NO: 168) |
| NGS ZooTsy- | gactggagttcagacgtgtgctctt |
| end1 (for | ccgatctctgcaagtaatgcgacat |
| upstream- | tgg |
| end1 | (SEQ ID NO: 169) |
| junction) | |
| NGS ZooTsy- | ctttccctacacgacgctcttccga |
| end1 (for | tctcctttatagtcagtgggttatc |
| circularized | cg |
| donor | (SEQID NO: 170) |
| junction) | |
| NGS KanR- | gactggagttcagacgtgtgctctt |
| cargo | ccgatctttcgagatcatgcatgag |
| upstream | ctcac |
| | (SEQ ID NO: 171) |
| NGS KanR- | gactggagttcagacgtgtgctctt |
| cargo | ccgatcttgaggatccaacatttcc |
| downstream | aatcactag |
| | (SEQ ID NO: 172) |
| pDonor | ctgcaagtaatgcgacattgg |
| (ZooTsy) | (SEQ ID NO: 173) |
| ddPCR | |
| for end1 | |
| pTarget | atcaaaactggtgaaactcacccag |
| ddPCR for | (SEQ ID NO: 174) |
| CmR1 | |
| pTarget | gtgttcacccttgttacaccgtttt |
| ddPCR for | c |
| CmR2 | (SEQ ID NO: 175) |

TABLE 9

| Probe | |
|---|---|
| description | Probe sequence |
| pTarget | tgcgttgatgcaattt |
| ddPCR TcR | ctatgcgcacccgt |
| | (SEQ ID NO: 176) |
| pDonor | tgtccacacccatgag |
| (CyCAST) | tggacaacttatgc |
| ddPCR | (SEQ ID NO: 177) |
| for RE | |
| pDonor | tcatgataagaaaaca |
| (AjTn6022) | ataggttgccactc |
| ddPCR | (SEQ ID NO: 178) |
| for RE | |
| pDonor | caacgaacaaggcaaa |
| (ZooTsy) | aattcggataaccc |
| ddPCR for | (SEQ ID NO: 179) |
| end1 | |
| pTarget | aggttttcaccgtaac |
| ddPCR CmR | acgccacatcttgc |
| | (SEQ ID NO: 180) |

### Purification of TniQ and TnsF Proteins

Each protein expression vector was transformed into BL21 (DE3) Competent Cells (NEB). A 4 mL of starter culture was grown in TB supplemented with 100 μg/ml ampicillin for 12 h, which was used to inoculate 2 L of TB for growth at 37° C. and 150 rpm until an OD600 of 0.6. Protein expression was induced by supplementation with IPTG to a final concentration of 0.5 mM. The cells were incubated at 16° C. for 16 h for protein expression, and then harvested by centrifugation for 20 min at 4° C. at 4000 rpm (Beckman Coulter Avanti J-E, rotor JLA9.100). All subsequent steps were performed at 4° C. Cell pellet was resuspended in 200 mL of lysis buffer (50 mM Tris-HCl, 500 mM NaCl, 5% glycerol, 1 mM DTT, pH 8.0) supplemented with complete Protease Inhibitor Cocktail (Millipore sigma 4693116001). Cells were disrupted by the LM20 Microfluidizer system at 28,000 PSI. Lysate was cleared by centrifugation for 30 min at 4° C. at 9000 rpm (Beckman Coulter Avanti J-E, rotor JLA-10.500). The cleared lysate was applied to 1 mL of packed Strep-Tactin Sepharose resin (IBA) and incubated with rotation for 1 h, followed by washing of the protein-bound beads in 50 mL of lysis buffer. The proteins were cleaved off by Ulp1 SUMO protease OR SENP at 4° C. for 16 h. Resulting TniQ proteins were concentrated by an Amicon Ultra Centrifugal Filter Units (Millipore), and its concentration were estimated by NuPAGE (Invitrogen) and eStain L1 Protein Staining System (GenScript). The concentrated protein was loaded onto a gel filtration column (Superdex 200 Increase 10/300 GL, GE Healthcare) equilibrated with storage buffer (50 mM Tris-HCl, 250 mM NaCl, 5% glycerol, 1 mM DTT, pH 8.0) via FPLC. The resulting fractions from gel filtration were analyzed and the fractions containing the protein were pooled, and stored at −80° C. To purify TwinStrep-bdSUMO-TnsF of AjTn6022, the bound bdSUMO-tagged proteins were eluted in 10 mL of lysis buffer supplemented with 2.5 mM desthiobiotin (Sigma) instead of digestion by SENP.

### Pull-Down Experiments for Detecting AjTn6022 TniQ Tnsl Interactions

TwinStrep-bdSUMO-TnsF was mixed with each purified TniQ protein in the assembly buffer (25 mM Tris-HCl, 250 mM NaCl, 1 mM DTT, pH 8.0) and incubated at 37° C. for 1 hour. The reaction was mixed with pre-washed Strep-Tactin Magnetic Microbeads (IBA) and further incubated at 4° C. for 30 min. The beads were washed three times with the wash buffer (25 mM Tris-HCl, 250 mM NaCl, 1 mM DTT, 0.05% Tween20, pH 8.0). Then, the protein complex on the beads were eluted and denatured in NuPAGE LDS sample buffer at 95° C. for 10 min. Samples were loaded on NuPAGE 4 to 12%, Bis-Tris Gel, separated by electrophoresis, and stained with Imperial Protein Stain (ThermoFisher Scientific).

### Electrophoretic Mobility Shift Assay (EMSA) for Detecting Tnsl-DNA Interactions

Purified PCR amplicons and oligos (HPLC purified, IDT) were used as DNA probes in the assay. For DNA-protein complex assembly, 20 ng DNA probe was mixed with purified AjTn6022 TnsF protein or ZooTsy nuclease-dead TnsF-Y584F mutant protein at different DNA: protein molecular ratio in assembly buffer (25 mM Tris-HCl, 50 mM NaCl, 1% glycerol, 5 mM MgCl₂, pH 8.0), and incubated at 37° C. for 1 hour. Samples were mixed with Novex Hi-Density TBE Sample Buffer (ThermoFischer Scientific) and loaded on pre-equilibrated Novex 6% TBE Gel (ThermoFischer Scientific) in 0.5X TBE buffer for electrophoresis. Gel images were captured by SYBR Gold-staining.

### Data and Code Availability

The Illumina NGS and Oxford Nanopore Technologies (ONT) sequencing data generated during this study are available at SRA (SRA: PRJNA913200). The code for transposition junction NGS reads analysis is provided on GitHub.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. An engineered CRISPR-associated transposon (CAST) system comprising:
(a) one or more Type I-D Cas proteins;
(b) one or more CRISPR-associated Tn7 transposases or functional fragments thereof linked to or otherwise capable of associating with the one or more Type I-D Cas proteins; and
(c) a guide molecule capable of forming a complex with the one or more Type I-D Cas proteins and directing sequence-specific binding of the complex to a target polynucleotide.

2. The engineered system of claim 1, wherein the one or more Type I-D Cas proteins comprise Cas5, Cas6, Cas7, and/or Cas10d, and optionally further comprise Cas1, Cas2, and/or Cas3d.

3. (canceled)

4. The engineered system of claim 1, wherein the one or more CRISPR-associated Tn7 transposases comprise (i) TnsA, TnsB, and TnsC; or (ii) TnsAB and TnsC, and optionally further comprise TniQ, TnsE, or TnsF.

5. (canceled)

6. (canceled)

7. (canceled)

8. The engineered system of claim 4, wherein the one or more CRISPR-associated Tn7 transposases further comprise a first TniQ, and a second TniQ, wherein the first TniQ and the second TniQ are different.

9. The engineered system of claim 8, wherein TniQ is derived from a first species, and the one or more Type I-D Cas proteins is derived from a second species different from the first species, or wherein the one or more CRISPR-associated Tn7 transposases are derived from a first species, and the one or more Type I-D Cas proteins are derived from a second species different from the first species.

10. (canceled)

11. The engineered system of claim 1, wherein the target polynucleotide comprises a protospacer adjacent motif (PAM), wherein the PAM optionally comprises the nucleotide sequence GTT.

12. (canceled)

13. The engineered system of claim 1, wherein the target polynucleotide comprises linear DNA, circular DNA, or genomic DNA.

14. The engineered system of claim 1, further comprising a plurality of guide molecules capable of forming a complex with the one or more Type I-D Cas proteins and directing sequence specific binding of the complex to one or more target polynucleotides.

15. A system comprising one or more polynucleotides encoding the components of claim 1.

16. The system of claim 15, further comprising a donor polynucleotide, wherein the donor polynucleotide optionally comprises a polynucleotide insert, a left element sequence, and a right element sequence.

17. (canceled)

18. A vector comprising one or more polynucleotides encoding the components of claim 1, and optionally further comprising a donor polynucleotide, wherein the donor polynucleotide optionally comprises a polynucleotide insert, a left element sequence, and a right element sequence.

19. (canceled)

20. (canceled)

21. An engineered cell comprising the system of claim 1, wherein the cell optionally produces and/or secretes an endogenous or non-endogenous biological product or chemical compound, wherein the biological product optionally comprises a protein or an RNA.

22. (canceled)

23. (canceled)

24. A cell line comprising the engineered cell of claim 21, or composition comprising the engineered cell of claim 21, wherein the composition is optionally formulated for use as a therapeutic.

25. (canceled)

26. (canceled)

27. A biological product or chemical compound produced by the engineered cell of claim 21, wherein the biological product optionally comprises a mutated protein or product provided by a template.

28. (canceled)

29. An engineered cell or progeny thereof, said cell or progeny thereof being engineered by use of the system of claim 1, wherein the cell or progeny thereof optionally produces and/or secretes an endogenous or non-endogenous biological product or chemical compound, wherein the biological product optionally comprises a protein or an RNA, and wherein the protein optionally comprises a mutation, and wherein the cell or progeny thereof optionally comprises a mutation in a protein expressed from a gene comprising the target sequence, and wherein the cell or progeny thereof optionally comprises a deletion of a genomic region comprising the target sequence, decreased transcription of a gene associated with the target sequence, or increased transcription of a gene associated with the target sequence.

30. (canceled)

31. (canceled)

32. (canceled)

33. (canceled)

34. (canceled)

35. (canceled)

36. (canceled)

37. A pharmaceutical composition for treatment of a disease or disorder, comprising the cell or progeny thereof of claim 29, wherein the treatment optionally results in genetic changes in one or more cells, correction of one or more defective genotypes, or improved phenotype.

38. (canceled)

39. (canceled)

40. (canceled)

41. (canceled)

42. (canceled)

43. (canceled)

44. (canceled)

45. A method of inserting a donor polynucleotide into a target polynucleotide in a cell, said method comprising introducing into the cell the system of claim 1, wherein the donor polynucleotide optionally:
(a) introduces one or more mutations to the target polynucleotide;
(b) corrects a premature stop codon in the target polynucleotide;
(c) disrupts a splicing site;
(d) restores a splicing site; or
(e) a combination thereof,
and wherein the mutations optionally comprise substitutions, deletions, insertions, or a combination thereof, and wherein the mutations optionally cause a shift in an open reading frame on the target polynucleotide.

46. (canceled)

47. (canceled)

48. (canceled)

49. (canceled)

50. The method of claim 45, wherein insertion of the donor polynucleotide into the target polynucleotide in the cell results in:
(a) a cell or population of cells comprising altered expression levels of one or more gene products; or
(b) a cell or population of cells that produces and/or secretes an endogenous or non-endogenous biological product or chemical compound,
and wherein the target polynucleotide optionally comprises linear DNA, circular DNA, or genomic DNA, and wherein the one or more components of the engineered system optionally is expressed from a nucleic acid operably linked to a regulatory sequence or is introduced into a particle for delivery into a cell.

51. (canceled)

52. (canceled)

53. (canceled)

54. An engineered system comprising one or more Tn7 transposases or functional fragments thereof comprising:
(a) TnsA, TnsB, TnsC, and TnsF; or
(b) TnsAB, TnsC, and TnsF.

55. The engineered system of claim 54, optionally further comprising TniQ, and optionally further comprising:
(a) one or more Cas proteins, wherein the Cas proteins optionally comprise a Type I, a Type II, or a Type V Cas protein, and optionally further comprise a catalytically inactivated Cas protein; and
(b) a guide molecule capable of forming a complex with the one or more Cas proteins and directing sequence-specific binding of the complex to a target polynucleotide, wherein the Tn7 transposases are optionally derived from a first species and the Cas proteins are optionally derived from a second species different from the first species.

56. (canceled)

57. (canceled)

58. (canceled)

59. (canceled)

60. (canceled)

61. A system comprising one or more polynucleotides encoding the components of claim 54, optionally further comprising a donor polynucleotide, wherein the donor polynucleotide optionally comprises a polynucleotide insert, a left element sequence, and a right element sequence.

62. (canceled)

63. (canceled)

64. A vector comprising one or more polynucleotides encoding the components of claim 54, and optionally further comprising a donor polynucleotide, wherein the donor polynucleotide optionally comprises a polynucleotide insert, a left element sequence, and a right element sequence.

65. (canceled)

66. (canceled)

67. An engineered cell comprising the system of any one of claim 54, or the vector of claim 64, wherein the cell optionally produces and/or secretes an endogenous or non-endogenous biological product or chemical compound, and wherein the biological product optionally comprises a protein or an RNA.

68. (canceled)

69. (canceled)

70. A cell line comprising the engineered cell of claim 67, or a composition comprising the engineered cell of claim 67, wherein the composition is optionally formulated for use as a therapeutic.

71. (canceled)

72. (canceled)

73. A biological product or chemical compound produced by the engineered cell of claim 67, wherein the biological product optionally comprises a mutated protein or product provided by a template.

74. (canceled)

75. An engineered cell or progeny thereof, said cell or progeny thereof being engineered by use of the system of any one of claim 54, wherein the cell or progeny thereof optionally produces and/or secretes an endogenous or non-endogenous biological product or chemical compound, wherein the biological product optionally comprises a protein or an RNA, wherein the protein optionally comprises a mutation, and wherein the cell or progeny thereof optionally is isolated or is further used as a therapeutic.

76. (canceled)

77. (canceled)

78. (canceled)

79. (canceled)

80. (canceled)

81. (canceled)

82. (canceled)

83. A pharmaceutical composition for treatment of a disease or disorder, comprising the cell or progeny thereof of claim 75, wherein the treatment optionally results in genetic changes in one or more cells, correction of one or more defective genotypes, or improved phenotype.

84. (canceled)

85. (canceled)

86. (canceled)

87. The engineered cell or progeny thereof of claim 75, wherein the cell or progeny thereof comprises a mutation in a protein expressed form from a gene comprising the target sequence, and wherein the cell or progeny thereof optionally comprises a deletion of a genomic region comprising the target sequence, decreased transcription of a gene associated with the target sequence, or increased transcription of a gene associated with the target sequence.

88. (canceled)

89. (canceled)

90. (canceled)

91. A method of inserting a donor polynucleotide into a target polynucleotide in a cell, said method comprising introducing into the cell the system of claim 54, wherein the donor polynucleotide optioinally:
(a) introduces one or more mutations to the target polynucleotide;
(b) corrects a premature stop codon in the target polynucleotide;
(c) disrupts a splicing site;
(d) restores a splicing site; or
e) a combination thereof,
wherein the one or more mutations optionally comprises substitutions, deletions, insertions, or a combination thereof, wherein the one or more mutations optionally causes a shift in an open reading frame on the target polynucleotide, and wherein the donor polynucleotide is optionally between 100 bases and 30 kilobases in length.

92. (canceled)

93. (canceled)

94. (canceled)

95. (canceled)

96. The method of claim 91, wherein insertion of the donor polynucleotide into the target polynucleotide in the cell results in:
(a) a cell or population of cells comprising altered expression levels of one or more gene products; or
(b) a cell or population of cells that produces and/or secretes an endogenous or non-endogenous biological product or chemical compound,
and wherein the target polynucleotide optionally comprises linear DNA, circular DNA, or genomic DNA, and wherein the one or more components of the engineered system optionally is expressed from a nucleic acid operably linked to a regulatory sequence or is introduced into a particle for delivery into a cell.

97. (canceled)

98. (canceled)

99. (canceled)

100. An engineered system comprising:
(a) one or more tyrosine recombinases;
(b) one or more helix-turn-helix (HTH) domain proteins; and
(c) one or more TnsF homologs comprising a catalytic nuclease domain.

101. The engineered system of claim 100, optionally further comprising one or more Cas proteins, wherein the Cas proteins optionally comprise a Type I, Type II, or Type V Cas protein, optionally comprise a catalytically inactivated Cas protein, and a guide molecule capable of forming a complex with the one or more Cas proteins and directing sequence-specific binding of the complex to a target polynucleotide, and optionally further comprising a plurality of guide molecules capable of forming a complex with the one or more Cas proteins and directing sequence-specific binding of the complex to one or more target polynucleotides, and optionally further comprising one or more GIY-YIG nucleases.

102. (canceled)

103. (canceled)

104. (canceled)

105. (canceled)

106. A system comprising one or more polynucleotides encoding the components of claim 100, and optionally further comprising a donor polynucleotide, wherein the donor polynucleotide optionally comprises a polynucleotide insert, a left element sequence, and a right element sequence.

107. (canceled)

108. (canceled)

109. A vector comprising one or more polynucleotides encoding the components of claim 100, and optionally further comprising a donor polynucleotide, wherein the donor polynucleotide optionally comprises a polynucleotide insert, a left element sequence, and a right element sequence.

110. (canceled)

111. (canceled)

112. An engineered cell comprising the system of claim 100, wherein the cell optionally produces and/or secretes an endogenous or non-endogenous biological product or chemical compound, and wherein the biological product optionally comprises a protein or an RNA.

113. (canceled)

114. (canceled)

115. A cell line comprising the engineered cell of claim 112, or a composition comprising the engineered cell of claim 112, wherein the composition is optionally formulated for use as a therapeutic.

116. (canceled)

117. (canceled)

118. A biological product or chemical compound produced by the engineered cell of claim 112, wherein the biological product optionally comprises a mutated protein or product provided by a template.

119. (canceled)

120. An engineered cell or progeny thereof, said cell or progeny thereof being engineered by use of the system of claim 100, wherein the cell or progeny thereof optionally produces and/or secretes an endogenous or non-endogenous biological product or chemical compound, wherein the biological product optionally comprises a protein or an RNA, wherein the protein optionally comprises a mutation, and wherein the cell or progeny thereof optionally is isolated or is further used as a therapeutic.

121. (canceled)

122. (canceled)

123. (canceled)

124. (canceled)

125. (canceled)

126. (canceled)

127. (canceled)

128. A pharmaceutical composition for treatment of a disease or disorder, comprising the cell or progeny thereof of claim 120, wherein the treatment optionally results in genetic changes in one or more cells, correction of one or more defective genotypes, or improved phenotype.

129. (canceled)

130. (canceled)

131. (canceled)

132. The engineered cell or progeny thereof of claim 120, wherein the cell or progeny thereof comprises a mutation in a protein expressed from a gene comprising the target sequence, and wherein the cell or progeny thereof optionally comprises a deletion of a genomic region comprising the target sequence, decreased transcription of a gene associated with the target sequence, or increased transcription of a gene associated with the target sequence.

133. (canceled)

134. (canceled)

135. (canceled)

136. A method of inserting a donor polynucleotide into a target polynucleotide in a cell, said method comprising introducing into the cell the system of claim 100, wherein the donor polynucleotide optionally:
(a) introduces one or more mutations to the target polynucleotide;
(b) corrects a premature stop codon in the target polynucleotide;
(c) disrupts a splicing site;
(d) restores a splicing site; or
(e) a combination thereof,
wherein the one or more mutations optionally comprises substitutions, deletions, insertions, or a combination thereof, wherein the one or more mutations optionally causes a shift in an open reading frame on the target polynucleotide, and wherein the donor polynucleotide is optionally between 100 bases and 30 kilobases in length.

137. (canceled)

138. (canceled)

139. (canceled)

140. (canceled)

141. The method of claim 136, wherein insertion of the donor polynucleotide into the target polynucleotide in the cell results in:
(a) a cell or population of cells comprising altered expression levels of one or more gene products; or
(b) a cell or population of cells that produces and/or secretes an endogenous or non-endogenous biological product or chemical compound,
and wherein the target polynucleotide optionally comprises linear DNA, circular DNA, or genomic DNA, and wherein the one or more components of the engineered system optionally is expressed from a nucleic acid operably linked to a regulatory sequence or is introduced into a particle for delivery into a cell.

142. (canceled)

143. (canceled)

144. (canceled)

145. (canceled)

146. (canceled)

147. (canceled)

148. (canceled)

149. (canceled)

150. (canceled)
